SEQUENCE LISTING (16 sequences)

SEQ ID NO: 1 - protein, 11 aa, synthetic construct
  REGION 1..11: note = Anti-HER2 antibody VL CDR 1 sequence
RASQDVNTAVA

SEQ ID NO: 2 - protein, 7 aa, synthetic construct
  REGION 1..7: note = Anti-HER2 antibody VL CDR 2 sequence
SASFLYS

SEQ ID NO: 3 - protein, 9 aa, synthetic construct
  REGION 1..9: note = Anti-HER2 antibody VL CDR 3 sequence
QQHYTTPPT

SEQ ID NO: 4 - protein, 5 aa, synthetic construct
  REGION 1..5: note = Anti-HER2 antibody VH CDR 1 sequence
DTYIH

SEQ ID NO: 5 - protein, 17 aa, synthetic construct
  REGION 1..17: note = Anti-HER2 antibody VH CDR 2 sequence
RIYPTNGYTRYADSVKG

SEQ ID NO: 6 - protein, 11 aa, synthetic construct
  REGION 1..11: note = Anti-HER2 antibody VH CDR 3 sequence
WGGDGFYAMDY

SEQ ID NO: 7 - protein, 107 aa, synthetic construct [sequence omitted]

SEQ ID NO: 8 - protein, 120 aa, synthetic construct [sequence omitted]

SEQ ID NO: 9-15 - skipped (7 entries)

SEQ ID NO: 16 - protein, 1254 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

(19) United States
(12) Patent Application Publication
(10) Pub. No.: US 2025/0205350 A1
(43) Pub. Date: Jun. 26, 2025

(54) Antibody-Drug Conjugates and Uses Thereof

(71) Applicant: Vivasor, Inc., San Diego, CA (US)

(72) Inventors: Yufeng Hong, San Diego, CA (US); Yanwen Fu, San Diego, CA (US); Zheng Yan, San Diego, CA (US)

(73) Assignee: Vivasor, Inc., San Diego, CA (US)

(21) Appl. No.: 18/844,284

(22) PCT Filed: Mar. 9, 2023
(86) PCT No.: PCT/US2023/064054

§ 371 Date: Sep. 5, 2024

Related U.S. Application Data
(60) Provisional application No. 63/318,693, filed on Mar. 10, 2022.

(51) Int. Cl. A61K 47/68; A61P 35/00; C07K 16/32

(52) U.S. Cl. CPC A61K 47/68037; A61K 47/6855; A61K 47/6889; A61P 35/00; C07K 16/32

(57) ABSTRACT

Provided, inter alia, are antibody drug conjugates (ADCs) comprising novel camptothecin derivative toxins. Further disclosed are pharmaceutical compositions, and methods for treating cancer using the ADCs provided herein.

This application claims the benefit of priority of U.S. Provisional Patent Application No. 63/318,693, filed Mar. 10, 2022, which is incorporated herein by reference in its entirety for all purposes.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure relates to novel camptothecin derivative compounds, camptothecin derivative-linker compounds, antibody drug conjugates (ADCs) comprising the novel camptothecin derivative toxins, and methods of preparing the same. Also provided herein are methods of treating cancer using the ADCs described herein.

### INTRODUCTION AND SUMMARY

Antibody-Drug Conjugates (ADCs) allow for the targeted delivery of a drug moiety to a tumor, and, in some embodiments intracellular accumulation therein, where systemic administration of unconjugated drugs may result in unacceptable levels of toxicity to normal cells (Polakis P. (2005) Current Opinion in Pharmacology 5:382-387). ADCs are targeted chemotherapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107.

The present disclosure provides ADCs comprising a monoclonal antibody conjugated to camptothecin derivative toxins through linker moieties. In embodiments, the monoclonal antibody is an anti-HER2 antibody. In embodiments, the anti-HER2 antibody binds to HER2-expressing cancer cells and allows for selective uptake of the ADC into the cancer cells. In embodiments, the ADCs provided herein selectively deliver an effective amount of the camptothecin derivative toxin to tumor tissue and reduce the non-specific toxicity associated with related ADCs. The ADC compounds described herein include those with anticancer activity.

Members of the ErbB family of transmembrane receptor tyrosine kinases are important mediators of cell growth, differentiation and survival. The receptor family includes four distinct members, including epidermal growth factor receptor (EGFR or ErbB1), HER2 (ErbB2 or p185^{neu}), HER3 (ErbB3) and HER4 (ErbB4 or tyro2). Both homo- and heterodimers are formed by the four members of the EGFR family, with HER2 being the preferred and most potent dimerization partner for other ErbB receptors (Graus-Porta et al., 1997, Embo 3(16):1647-1655; Tao et al., 2008, J. Cell Sci. 121:3207-3217). HER2 has no known ligand, but can be activated via homodimerization when overexpressed, or by heterodimerization with other, ligand occupied ErbB receptors.

The HER2 gene is amplified in 20-30% of early-stage breast cancers, which makes such cancers overexpress epidermal growth factor (EGF) receptors in the cell membrane (Bange, et al., Nature Medicine 7 (5): 548-552). Besides breast cancer, HER2 expression has also been associated with other human carcinoma types, including non-small cell lung cancer, ovarian cancer, gastric cancer, prostate cancer, bladder cancer, colon cancer, esophageal cancer and squamous cell carcinoma of the head & neck (Garcia de Palazzo et al., 1993, Int. J. Biol. Markers 8:233-239; Ross et al., 2003, Oncologist 8:307-325; Osman et al., 2005, J. Urol. 174:2174-2177; Kapitanovic et al., 1997, Gastroenterology 112:1103-1113; Turken et al., 2003, Neoplasma 50:257-261; and Oshima et al., 2001, Int. J. Biol. Markers 16:250-254).

Camptothecin (CPT) is a cytotoxic quinoline alkaloid isolated from Camptotheca acuminta, a type of tree natively growing in China. CPT was discovered in the 1960s (Wall M. E. et al., 1966, J. Am. Chem. Soc. 88:3888-3890). The antitumor activity of Camptothecin depends on a highly specific inhibition of Topoisomerase-I (TOPO 1). The enzyme TOPO 1 cleaves one strand of double stranded DNA, partially unwinds the DNA, and then reanneals the strand to relieve tension. Camptothecin and its derivatives bind to the TOPO 1/DNA complex to prevent reannealing, which can cause cell death due to the accumulation of partially cleaved DNA (Hsiang Y. H., et al, 1985, J. Biol. Chem. 260:14873-14878).

The clinical application of camptothecin is limited due to its low solubility as well as serious side-effects (Joerger M. et al., 2015, Br. J. Clin. Pharmacol. 80:128-138; Joerger M. et al., 2015, Invest. New Drugs 33:472-479). To overcome these drawbacks, several camptothecin derivatives have been developed to date, including topotecan (9-dimethyl amino-10-hydroxy camptothecin; TPT) and irinotecan (7-ethyl-10-[4-(1-piperidino)-1-piperidino]carbonyloxycamptothecin; CPT-11) (Naumczuk B. et al., 2017, Magn. Reason. Chem. 55:128-136; Hamilton G. et al., 2014, Molecules 19:2077-2088). The US Food and Drug Administration has approved these CPT derivatives for ovarian and colon cancer treatment (Vladu et al., 2000, Mol. Pharmacol. 57:243-251; Chazin et al., 2014, Mini Rev. Med. Chem. 14:953-962).

Another camptothecin derivative is exatecan, which is a water soluble derivative of camptothecin (U.S. Pat. Nos. 10,195,288, 8,575,188). Unlike irinotecan currently used in clinical settings, an activation by an enzyme is unnecessary. Dxd is another useful camptothecin derivative.

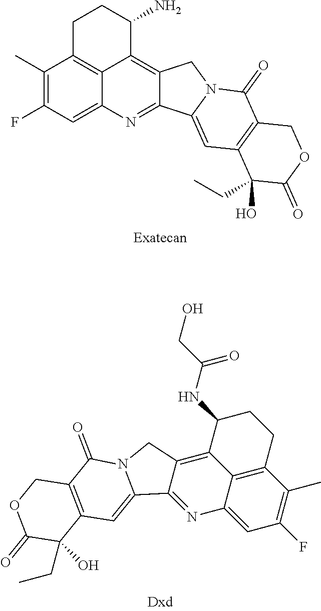 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3]

Many camptothecin drugs are widely applied clinically, and the main indications are bone cancer, prostatic cancer, breast cancer, gastric cancer, pancreatic cancer, ovarian cancer, esophageal cancer, endometrial cancer and the like (Igbal et al., 2014, Mol. Biol. Int. 2014). Camptothecin drugs have a short half-life in plasma and maintaining drug efficacy in clinical use requires an increased dose or increased frequency of administration, thus possibly causing tolerance problems to patients. Accordingly, there exists a need for improved camptothecin drugs.

In one aspect, provided herein are antibody-drug conjugates (ADCs) comprising a monoclonal antibody. In another aspect, provided herein are methods of preparing ADCs comprising a monoclonal antibody. In another aspect, provided herein are methods for treating cancers, such as HER2-expressing cancers, using the ADCs disclosed herein. Also, provided herein are novel drug-linker compounds.

In embodiments, the present disclosure provides an antibody drug conjugate (ADC), having an IgG antibody that binds to a HER2 target, conjugated at one or more cysteine sites of the IgG antibody. In embodiments, the present disclosure provides an antibody drug conjugate (ADC), having an IgG antibody that binds to a HER2 target, conjugated at one or more lysine sites of the IgG antibody. In embodiments, the present disclosure provides an antibody drug conjugate (ADC), having a modified IgG antibody that binds to a HER2 target. The present disclosure further provides a method for treating breast cancer, metastatic breast cancer or non-small-cell lung cancer comprising providing an effective amount of a HER2 ADC.

In one aspect, provided herein is an antibody drug conjugate (ADC) of formula (I)

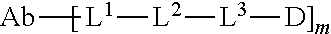

or formula (II)

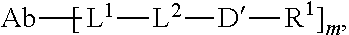

or a pharmaceutically acceptable salt thereof, wherein Ab is a monoclonal antibody; m is an integer from 1 to 8; L¹ is a linker bound to the monoclonal antibody; L² is a bond, —C(O)—, —NH—, Amino Acid Unit, —(CH₂CH₂O)ₙ—, —(CH₂)ₙ—, -(4-aminobenzyloxycarbonyl)-, —(C(O)CH₂CH₂NH)—, —(C(O)N(R²)CH₂CH₂N(R³))—, —O—, or any combination thereof, wherein n is an integer from 1 to 24; each R² and R³ is independently H or substituted or unsubstituted alkyl; L³ is a substituted or unsubstituted heterocycloalkylene or a substituted or unsubstituted heteroarylene; substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²; R¹ is a substituted or unsubstituted heterocycloalkyl or a substituted or unsubstituted heteroaryl; D is

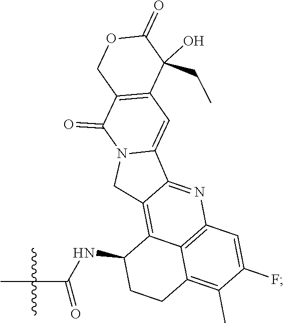 [SMILES: *C(=O)N[C@@H]1CCc2c(C)c(F)cc3nc4c(c1c23)Cn1c-4cc2c(c1=O)COC(=O)[C@@]2(O)CC]

### and D′ is

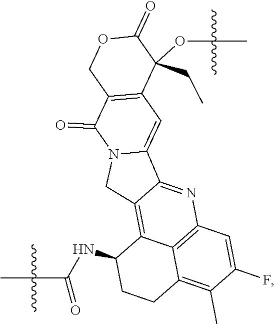

wherein D′ is connected through its amide group to R¹, and through oxygen to L².

In an aspect, provided herein is a method of treating a HER2-expressing cancer in a subject in need thereof, said method including administering the ADC described herein (including in an aspect, embodiment, table, example, or claim), or a pharmaceutically acceptable salt thereof, to the subject.

In an aspect, provided herein is a method of preparing an antibody drug conjugate (ADC) of formula (I)

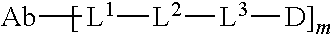

or formula (II)

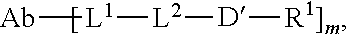

or a pharmaceutically acceptable salt thereof, said method including reacting an anti-HER2 antibody, or a modified antibody with a molecule of formula (P-I) B-L²-L³-D or formula (P-II) B-L²-D′-R¹, or a pharmaceutically acceptable salt thereof, wherein B is a reactive moiety capable of forming a bond with a monoclonal antibody; L² is a bond, —C(O)—, —NH—, Amino Acid Unit, —(CH₂CH₂O)ₙ—, —(CH₂)—, -(4-aminobenzyloxycarbonyl)-, —O—, —(C(O)CH₂CH₂NH)—, —(C(O)N(R²)CH₂CH₂N(R³))—, or any combination thereof, wherein n is an integer from 1 to 24; each R² and R³ is independently H or substituted or unsubstituted alkyl; L³ is a substituted or unsubstituted heterocycloalkylene, substituted or unsubstituted heteroarylene, substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²; R¹ is a substituted or unsubstituted heterocycloalkyl or a substituted or unsubstituted heteroaryl; D is

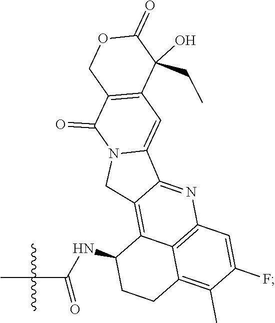 [SMILES: *C(=O)N[C@@H]1CCc2c(C)c(F)cc3nc4c(c1c23)Cn1c-4cc2c(c1=O)COC(=O)[C@@]2(O)CC]

### and D′ is

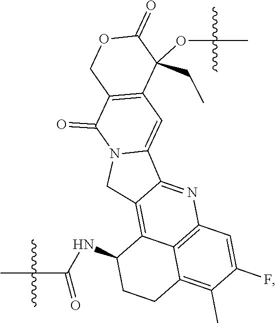

wherein D′ is connected through its amide group to R¹, and through oxygen to L².

In another aspect, provided herein is a compound of formula (III):

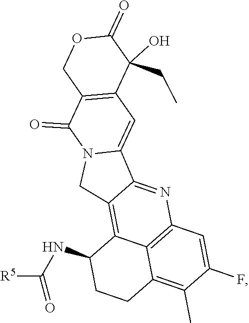

or a pharmaceutically acceptable salt, solvate, hydrate, stereoisomer, or prodrug thereof, wherein R⁵ is a substituted or unsubstituted heterocycloalkyl, substituted or unsubstituted heteroaryl, substituted or unsubstituted —CH₂NCH₂-(heteroaryl), substituted or unsubstituted —CH₂NCH₂-(heterocycloalkyl), substituted or unsubstituted —OCH₂-(heterocycloalkyl), or substituted or unsubstituted —OCH₂-(heteroaryl).

In an aspect, provided herein is a pharmaceutical composition comprising the ADC described herein, or a pharmaceutically acceptable salt thereof, and a pharmaceutically acceptable excipient.

In any embodiment disclosed herein, the monoclonal antibody can be an anti-HER2 antibody.

### BRIEF DESCRIPTION OF THE DRAWINGS

FIG. 1 shows results of an in vitro efficacy study of camptothecin derivatives in: SkBr-3 (HER2+) cells (FIG. 1A) and MDA-MB-468 (HER2−) cells (FIG. 1B).

FIG. 2 shows the chemical structures of camptothecin derivatives used in the in vitro efficacy study (see FIG. 1A and FIG. 1B).

FIG. 3 shows results of an in vitro efficacy study of anti-HER2 antibody linked camptothecin derivatives (ADCs) in: SkBr-3 (HER2+) cells (FIG. 3A) and MDA-MB-468 (HER2−) cells (FIG. 3B).

FIG. 4 shows results of an in vitro efficacy study of anti-HER2 antibody linked camptothecin derivatives (ADCs) in: SkBr-3 (HER2+) cells (FIG. 4A) and MDA-MB-468 (HER2−) cells (FIG. 4B).

FIG. 5 shows results of an in vitro efficacy study of camptothecin derivatives in: SkBr-3 (HER2+) cells (FIG. 5A) and MDA-MB-468 (HER2−) cells (FIG. 5B).

FIG. 6 shows the chemical structures of camptothecin derivatives used in the in vitro efficacy study (see FIG. 5A and FIG. 5B).

FIG. 7 shows results of an in vitro efficacy study of anti-HER2 antibody linked camptothecin derivatives (ADCs) in: SkBr-3 (HER2+) cells (FIG. 7A), MDA-MB-468 (HER2−) cells (FIG. 7B), and NCI-N87 (HER2+) cells (FIG. 7C).

FIG. 8 shows results of an in vivo efficacy study in NCI-N87 xenograft in Nu/Nu nude mice of anti-HER2 antibody linked camptothecin derivatives (ADCs), where the mice were treated once intravenously with either 3 mg/kg or 10 mg/kg of ADC (or a control). FIG. 8A displays tumor volume as a function of time. FIG. 8B top graph displays tumor volume as a function of time of selected ADC treatments from FIG. 8A (only 3 mg/kg treatment). Bottom graph displays tumor volume as a function of time of selected ADC treatments from FIG. 8A. FIG. 8C displays percent change in tumor volume as a function of time (same experiment of FIG. 8A).

FIG. 9 shows results of an in vivo efficacy study in NCI-N87 xenograft in Nu/Nu nude mice of anti-HER2 antibody linked camptothecin derivatives (ADCs), where the mice were treated once intravenously with either 3 mg/kg or 10 mg/kg of ADC (or a control). FIG. 9A displays tumor volume as a function of time. FIG. 9B displays percent change in tumor volume as a function of time (same experiment of FIG. 9A).

### DETAILED DESCRIPTION OF THE INVENTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In embodiments, about includes the specified value.

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “polypeptide,” “peptide” and “protein” and other related terms used herein are used interchangeably to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. Polypeptides include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 3 polypeptide chains) can associate with each other, via covalent and/or non-covalent association, to form a multimeric polypeptide complex (e.g., multi-specific antigen binding protein complex). Association of the polypeptide chains can also include peptide folding. Thus, a polypeptide complex can be dimeric, trimeric, tetrameric, or higher order complexes depending on the number of polypeptide chains that form the complex.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

The term “cancer” refers to all types of cancer, neoplasm or malignant tumors found in mammals (e.g. humans), including leukemias, lymphomas, carcinomas and sarcomas. In embodiments, the ADCs and methods provided herein are useful for treating HER2-expressing cancers. In embodiments, the HER2-expressing cancer is a solid tumor. The cancer may be any cancer in which an abnormal number of blast cells or unwanted cell proliferation is present or that is diagnosed as breast cancer, including metastatic breast cancer; gastric cancer; esophageal cancer, including squamous cell carcinomas and especially adenocarcinomas; ovarian cancer, including epithelial ovarian cancer; endometrial cancer, including endometrial carcinomas such as endometrial serous carcinoma; or lung cancer, including lung adenocarcinomas and non-small cell lung cancer.

The HER2 protein is overexpressed in various human tumors and can be evaluated using a method generally carried out in the art, such as an immunohistochemical staining method (IHC) for evaluating the overexpression of the HER2 protein, or a fluorescence in situ hybridization method (FISH) for evaluating amplification of the HER2 gene. Further, the anti-HER2 antibody-drug conjugate of the present invention exhibits an antitumor effect by recognizing, through its anti-HER2 antibody, the HER2 protein expressed on the surface of cancer cells and HER2 protein internalized in the cancer cells. Thus, the treatment subject of the anti-HER2 antibody-drug conjugate of the present invention is not limited to the “cancer expressing HER2 protein on the surface of the cancer cell” and can also be, for example, leukemia, malignant lymphoma, plasmacytoma, myeloma, or sarcoma (where HER2 protein is internalized in the cancer cells).

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

Exemplary cancers that may be treated with an ADC or method provided herein include breast cancer, non-small cell lung cancer, ovarian cancer, gastric cancer, kidney cancer, cervical cancer, prostate cancer, bladder cancer, ductal cancer, pancreatic cancer, colon cancer, colorectal cancer, urothelial cancer, salivary gland cancer, brain cancer, esophageal cancer and squamous cell carcinoma of the head & neck, or metastases of aforementioned cancers. In a more specific embodiment, the breast cancer is estrogen receptor and progesterone receptor negative breast cancer or triple negative breast cancer (TNBC). In another embodiment, the lung cancer is non-small cell lung cancer (NSCLC).

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Anti-HER2 antibodies are described herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Anti-HER2 antibodies, and antigen binding proteins thereof, that bind an epitope of a HER2 polypeptide are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. Antigen-binding fragments of anti-HER2 antibodies are described herein.

An antigen binding protein can have, for example, the structure of an immunoglobulin. In one embodiment, an “immunoglobulin” refers to a tetrameric molecule. Each tetrameric molecule is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab. “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). Antigen binding proteins having immunoglobulin-like properties that bind specifically to HER2 are described herein.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The terms “antigen binding protein” “antigen binding domain,” “antigen binding region,” or “antigen binding site” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins that bind HER2 are described herein.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

“Specifically binds” as used throughout the present specification in relation to anti-HER2 antigen binding proteins means that the antigen binding protein binds human HER2 (hHER2) with no or insignificant binding to other human proteins. The term however does not exclude the fact that antigen binding proteins of the invention may also be cross-reactive with other forms of HER2, for example primate HER2. In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

The term “HER2,” as used herein, refers to any native HER2 from any vertebrate source, including mammals such as primates (e.g. humans, cynomolgus monkey (cyno)) and rodents (e.g., mice and rats), unless otherwise indicated. The term encompasses “full-length,” unprocessed HER2 as well as any form of HER2 that results from processing in the cell. The term also encompasses naturally occurring variants of HER2, e.g., splice variants, allelic variants, and isoforms. The amino acid sequence of an exemplary human HER2 protein is shown in SEQ ID NO: 16.

The term “HER2-expressing cancer” refers to a cancer comprising cells that express HER2 on their surface. In embodiments, the term “HER2-expressing cancer” refers to a cancer comprising cells that internalize HER2 inside the cells.

The terms “anti-HER2 antibody” and “an antibody that binds to HER2” refer to an antibody that is capable of binding HER2 with sufficient affinity such that the antibody is useful as a therapeutic agent in targeting HER2. In one embodiment, the extent of binding of an anti-HER2 antibody to an unrelated, non-HER2 protein is less than about 10% of the binding of the antibody to HER2 as measured, e.g., by a radioimmunoassay (RIA). In certain embodiments, an antibody that binds to HER2 has a dissociation constant (Kd) of ≤1 μM, ≤100 nM, ≤10 nM, ≤5 nM, ≤4 nM, ≤3 nM, ≤2 nM, ≤nM, ≤0.1 nM, ≤0.01 nM, or 0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-HER2 antibody binds to an epitope of HER2 that is conserved among HER2 from different species.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the anti-HER2 antibodies described herein.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc” or “Fc region” as used herein refers to the portion of an antibody heavy chain constant region beginning in or after the hinge region and ending at the C-terminus of the heavy chain. The Fc region comprises at least a portion of the CH and CH3 regions, and may or may not include a portion of the hinge region. Two polypeptide chains each carrying a half Fc region can dimerize to form an Fc region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc region can bind an Fc receptor, including FcγRI (e.g., CD64), FcγRII (e.g, CD32) and/or FcγRIII (e.g., CD16a).

“Humanized antibody” refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human anti-HER2 antibodies and antigen binding proteins thereof are described herein. This definition of a human antibody specifically excludes a humanized antibody comprising non-human antigen-binding residues.

The term “isolated”, means altered “by the hand of man” from its natural state, has been changed or removed from its original environment, or both. When the term “isolated” is applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis, high-performance liquid chromatography or mass spectrophotometry. A protein that is the predominant species present in a preparation is substantially purified. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell, even if the cell is of the same species or type as that from which the polynucleotide or polypeptide was separated.

“CDRs” are defined as the complementarity determining region amino acid sequences of an antibody which are the hypervariable domains of immunoglobulin heavy and light chains. There are three heavy chain and three light chain CDRs (or CDR regions) in the variable portion of an immunoglobulin. Thus, “CDRs” as used herein may refer to all three heavy chain CDRs, or all three light chain CDRs (or both all heavy and all light chain CDRs, if appropriate).

CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

The CDR sequences of antibodies can be determined by the Kabat numbering system (Kabat et al; (Sequences of proteins of Immunological Interest NIH, 1987); alternatively they can be determined using the Chothia numbering system (Al-Lazikani et al., (1997) JMB 273, 927-948), the contact definition method (MacCallum R. M., and Martin A. C. R. and Thornton J. M, (1996), Journal of Molecular Biology, 262 (5), 732-745) or any other established method for numbering the residues in an antibody and determining CDRs known to the skilled in the art.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein the term “domain” refers to a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. An “antibody single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “cytotoxic agent” as used herein refers to a substance that inhibits or prevents a cellular function and/or causes cell death or destruction. Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “chemotherapeutic agent” is a chemical compound useful in the treatment of a cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®; Bristol-Myers Squibb Oncology, Princeton, N.J.), ABRAXANE™ Cremophor-free, albumin-engineered nanoparticle formulation of paclitaxel (American Pharmaceutical Partners, Schaumberg, Illinois), and docetaxel (TAXOTERE®; Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone; CVP, an abbreviation for a combined therapy of cyclophosphamide, vincristine, and prednisolone; and FOLFOX, an abbreviation for a treatment regimen with oxaliplatin (ELOXATIN™) combined with 5-FU and leucovorin.

An “antibody-drug conjugate” or “ADC” is an antibody conjugated to one or more heterologous molecule(s), including but not limited to a cytotoxic agent.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In certain embodiments, the subject is an adult, an adolescent, a child, or an infant. In some embodiments, the terms “individual” or “patient” are used and are intended to be interchangeable with “subject”.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (e.g., EMBOSS Needle or EMBOSS Water, available at www.ebi.ac.uk/Tools/psa/). Those skilled in the art can determine appropriate parameters for aligning sequences, including any algorithms needed to achieve maximal alignment over the full length of the sequences being compared. “Percentage of sequence identity” or “percent (%) [sequence] identity”, as used herein, is determined by comparing two optimally locally aligned sequences over a comparison window defined by the length of the local alignment between the two sequences. (This may also be considered percentage of homology or “percent (%) homology”.) The amino acid sequence in the comparison window may comprise additions or deletions (e.g., gaps or overhangs) as compared to the reference sequence for optimal alignment of the two sequences. Local alignment between two sequences only includes segments of each sequence that are deemed to be sufficiently similar according to a criterion that depends on the algorithm used to perform the alignment (e.g., EMBOSS Water). “identical” or percent “identity,” refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region). The percentage identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (Add. APL. Math. 2:482, 1981), by the global homology alignment algorithm of Needleman and Wunsch (J. Mol. Biol. 48:443, 1970), by the search for similarity method of Pearson and Lipman (Proc. Natl. Acad. Sci. USA 85: 2444, 1988), or by inspection. GAP and BESTFIT, as additional examples, can be employed to determine the optimal alignment of two sequences that have been identified for comparison. Typically, the default values of 5.00 for gap weight and 0.30 for gap weight length are used.

A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not identical to any of the amino acid sequences of the polypeptides that make up the multi-specific antigen binding protein complexes described herein. The similarities between the test antibody and the polypeptides can be at least 95%, or at or at least 96% identical, or at least 97% identical, or at least 98% identical, or at least 99% identical, to any of the polypeptides that make up the multi-specific antigen binding protein complexes described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “pharmaceutical formulation” refers to a preparation which is in such form as to permit the biological activity of an active ingredient contained therein to be effective, and which contains no additional components which are unacceptably toxic to a subject to which the formulation would be administered.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “effective amount” of an agent, e.g., a pharmaceutical formulation, refers to an amount effective, at dosages and for periods of time necessary, to achieve the desired therapeutic or prophylactic result.

The abbreviations used herein have their conventional meaning within the chemical and biological arts. The chemical structures and formulae set forth herein are constructed according to the standard rules of chemical valency known in the chemical arts.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Where substituent groups are specified by their conventional chemical formulae, written from left to right, they equally encompass the chemically identical substituents that would result from writing the structure from right to left, e.g., —CH₂O— is equivalent to —OCH₂—.

The term saccharide means carbohydrate (or sugar). In embodiments, the saccharide is a monosaccharide. In embodiments, the saccharide is a polysaccharide. The most basic unit of saccharide is a monomer of carbohydrate. The general formula is CₙH₂ₙOₙ. The term saccharide derivative means sugar molecules that have been modified with substituents other than hydroxyl groups. Examples include glycosylamines, sugar phosphates, and sugar esters. Other saccharide derivatives include for example beta-D-glucuronyl, D-galactosyl, and D-glucosyl.

The term “Charged Group” means a chemical group bearing a positive or a negative charge, such as for example phosphate, phosphonate, sulfate, sulfonate, nitrate, carboxylate, carbonate, etc. In some embodiments, a Charged Group is at least 50% ionized in aqueous solution at least one pH in the range of 5-9. In some embodiments, a Charged Group is an anionic Charged Group.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in addition to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in addition to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” (a wavy line) denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

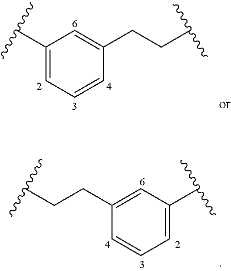 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, =N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)ₚ—U—, wherein T and U are independently —NR—, —O—, —CRR′—, or a single bond, and p is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Linker” refers to a chemical moiety comprising a covalent bond or a chain of atoms that covalently attaches an antibody to a drug moiety. In various embodiments, linkers include a divalent radical. In various embodiments, linkers can comprise one or more amino acid residues. In embodiments, the linker is a non-cleavable linker. In embodiments, the linker is an enzyme-cleavable linker (e.g., Val-Cit or Val-Cit-PAB linker).

“Amino Acid Unit” has the formula

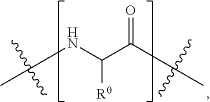

where R⁰ is hydrogen, methyl, isopropyl, isobutyl, sec-butyl, benzyl, p-hydroxybenzyl, CH₂OH, CH(OH)CH₃, —CH₂CH₂SCH₃, CH₂CONH₂, CH₂COOH, CH₂CH₂CONH₂, CH₂CH₂COOH, (CH₂)₃NHC(═NH)NH₂, (CH₂)₃NH₂, (CH₂)₃NHCOCH₃, (CH₂)₃NHCHO, (CH₂)₄NHC(═NH)NH₂, (CH₂)₄NH₂, (CH₂)₄NHCOCH₃, (CH₂)₄NHCHO, (CH₂)₃NHCONH₂, (CH₂)₄NHCONH₂, CH₂CH₂CH(OH)CH₂NH₂, 2-pyridylmethyl-, 3-pyridylmethyl-, 4-pyridylmethyl-, phenyl, or cyclohexyl. In various embodiments, Amino Acid Unit includes not only naturally occurring amino acids but also minor amino acids, and non-naturally occurring amino acid analogs, such as citrulline, norleucine, selenomethionine, β-alanine, etc. An amino acid unit may be referred to by its standard three-letter code for the amino acid (e.g., Ala, Cys, Asp, Glu, Val, Phe, Lys, etc.).

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., thiol, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a thiol). In embodiments, the first bioconjugate reactive group (e.g., haloacetyl moiety) is covalently attached to the second bioconjugate reactive group (e.g. a thiol). In embodiments, the first bioconjugate reactive group (e.g., pyridyl moiety) is covalently attached to the second bioconjugate reactive group (e.g. a thiol). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., fluorophenyl ester moiety) reacts with the second bioconjugate reactive group (e.g. an amine) to form a covalent bond. In embodiments, the first bioconjugate reactive group (e.g., -sulfo-N-hydroxysuccinimide moiety) reacts with the second bioconjugate reactive group (e.g. an amine) to form a covalent bond.

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or thiol groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a thiol group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, common organic and cell types abbreviations are defined as follows:
Ac Acetyl
ACN Acetonitrile
Ala Alanine
Asn Asparagine
aq. Aqueous
β-Ala beta-alanine
BOC or Boc tert-Butoxycarbonyl
° C. Temperature in degrees Centigrade
CBZ Benzoxycarbonyl
Cit Citrulline
DBU 1,8-Diazabicyclo[5.4.0]undec-7-ene
DCM dichloromethane
DIEA Diisopropylethylamine
DMAP % % 4-(Dimethylamino)pyridine
DMF N,N-Dimethylformamide
DMSO % % Dimethyl sulfoxide
EDC 1-Ethyl-3-(3-dimethylaminopropyl)carbodiimide
EEDQ N-ethoxycarbonyl-2-ethoxy-1,2-dihydroquinoline
Et Ethyl
EtOAc Ethyl acetate
Eq Equivalents
Fmoc 9-Fluorenylmethoxycarbonyl
g Gram(s)
Gly Glycine
hr Hour (hours)
HATU 2-(1H-7-azabenzotriazol-1-yl)-1,1,3,3-tetramethyl uronium Hexafluorophosphate
HOBt N-Hydroxybenzotriazole
HPLC High-performance liquid chromatography
LC/MS Liquid chromatography-mass spectrometry
Lys Lysine
Me Methyl
mg milligrams
MeOH Methanol
mL Milliliter(s)
μL/μL Microliter(s)
mol moles
mmol millimoles
μmol/umol micromoles
MS mass spectrometry
NHS N-Hydroxysuccinimide
PAB or PABC p-aminobenzyloxycarbonyl
Phe Phenylalanine
Pip piperidine
PyAOP (7-Azabenzotriazol-1-yloxy)tripyrrolidinophosphonium hexafluorophosphate
RP-HPLC reverse phase HPLC
rt room temperature
Ser Serine
t-Bu tert-Butyl
Tert, t-tertiary
TFA Trifluoracetic acid
Thr Threonine
Val Valine

### Compositions

### Antibody-Drug Conjugates

In one aspect, provided herein is an antibody-drug conjugate (ADC) comprising a monoclonal antibody (Ab), a drug moiety (D), and a linker moiety that covalently attaches the monoclonal antibody to the drug moiety.

In another aspect, provided herein is an ADC of formula (I) or formula (II):

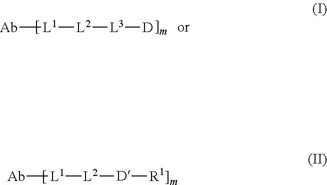

or a pharmaceutically acceptable salt thereof, wherein:
Ab is a monoclonal antibody;
m is an integer from 1 to 8;
L¹ is a linker bound to the monoclonal antibody;
L² is a bond, —C(O)—, —NH—, Amino Acid Unit, —(CH₂CH₂O)ₙ—, —(CH₂)ₙ—, —O—, -(4-aminobenzyloxycarbonyl)-, —(C(O)CH₂CH₂NH)—, —(C(O)N(R²)CH₂CH₂N(R³))—, or any combination thereof, wherein n is an integer from 1 to 24;
each R² and R³ is independently H or substituted or unsubstituted alkyl;
L³ is a substituted or unsubstituted heterocycloalkylene, substituted or unsubstituted heteroarylene, substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²;
R¹ is a substituted or unsubstituted heterocycloalkyl or a substituted or unsubstituted heteroaryl;
D is

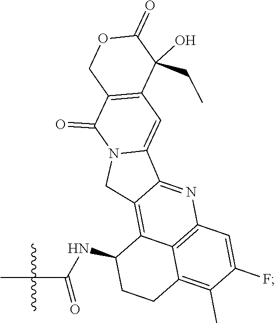 [SMILES: *C(=O)N[C@@H]1CCc2c(C)c(F)cc3nc4c(c1c23)Cn1c-4cc2c(c1=O)COC(=O)[C@@]2(O)CC]

and D′ is

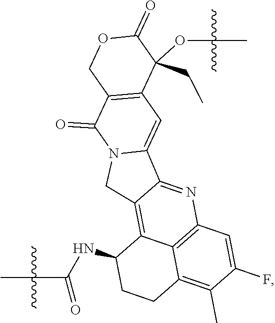

wherein D′ is connected through its amide group to R¹, and through oxygen to L².

In embodiments, m is an integer from 1 to 8. In embodiments, m is 1. In embodiments, m is 2. In embodiments, m is 3. In embodiments, m is 4. In embodiments, m is 5. In embodiments, m is 6. In embodiments, m is 7. In embodiments, m is 8.

In embodiments, n is an integer from 1 to 24. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24.

In embodiments, the monoclonal antibody is an anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody. In embodiments, the monoclonal antibody is an anti-HER2 antibody. In embodiments, the monoclonal antibody is an anti-ROR1 antibody. In embodiments, the monoclonal antibody is an anti-CD25 antibody. In embodiments, the monoclonal antibody is an anti-TROP2 antibody. In embodiments, the monoclonal antibody is an anti-B7-H3 antibody. In embodiments, the monoclonal antibody is an anti-c-Met antibody. In embodiments, the monoclonal antibody is an anti-FOLR1 antibody. In embodiments, the monoclonal antibody is an anti-CHOP2 antibody. In embodiments, the monoclonal antibody binds a transmembrane protein, e.g., an extracellular domain of a transmembrane protein. In embodiments, the transmembrane protein is a transmembrane receptor, such as a transmembrane receptor kinase. In embodiments, the transmembrane receptor kinase is a transmembrane receptor tyrosine kinase. In embodiments, the monoclonal antibody binds a tyrosine kinase.

In embodiments, the monoclonal antibody is a modified antibody. In embodiments, the monoclonal antibody is a modified anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody. In embodiments, the modified antibody binds a transmembrane protein, e.g., an extracellular domain of a transmembrane protein. In embodiments, the transmembrane protein is a transmembrane receptor, such as a transmembrane receptor kinase. In embodiments, the transmembrane receptor kinase is a transmembrane receptor tyrosine kinase. In embodiments, the modified antibody binds a tyrosine kinase.

In embodiments, L¹ is a linker bound to the monoclonal antibody. In embodiments, L¹ is a linker bound to one or two sulfur or nitrogen atoms on the monoclonal antibody. In embodiments, L¹ is a linker bound to one sulfur atom on the monoclonal antibody. In embodiments, L¹ is a linker bound to two sulfur atoms on the monoclonal antibody. In embodiments, L¹ is a linker bound to one nitrogen atom on the monoclonal antibody. In embodiments, L¹ is a linker bound to two nitrogen atoms on the monoclonal antibody.

In embodiments, L¹ is a linker bound to a modified monoclonal antibody.

In embodiments, L¹ is a linker bound to the anti-HER2 antibody. In embodiments, L¹ is a linker bound to one or two sulfur or nitrogen atoms on the anti-HER2 antibody. In embodiments, L¹ is a linker bound to one sulfur atom on the anti-HER2 antibody. In embodiments, L¹ is a linker bound to two sulfur atoms on the anti-HER2 antibody. In embodiments, L¹ is a linker bound to one nitrogen atom on the anti-HER2 antibody. In embodiments, L¹ is a linker bound to two nitrogen atoms on the anti-HER2 antibody.

In embodiments, L¹ is a linker bound to one cysteine molecule on the anti-HER2 antibody. In embodiments, L¹ is a linker bound to two cysteine molecules on the anti-HER2 antibody. In embodiments, L¹ is a linker bound to one lysine molecule on the anti-HER2 antibody. In embodiments, L¹ is a linker bound to two lysine molecules on the anti-HER2 antibody.

In embodiments, L¹ is a linker bound to a modified anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody. In embodiments, L¹ is a linker bound to a modified anti-HER2 antibody.

In embodiments, L¹ is

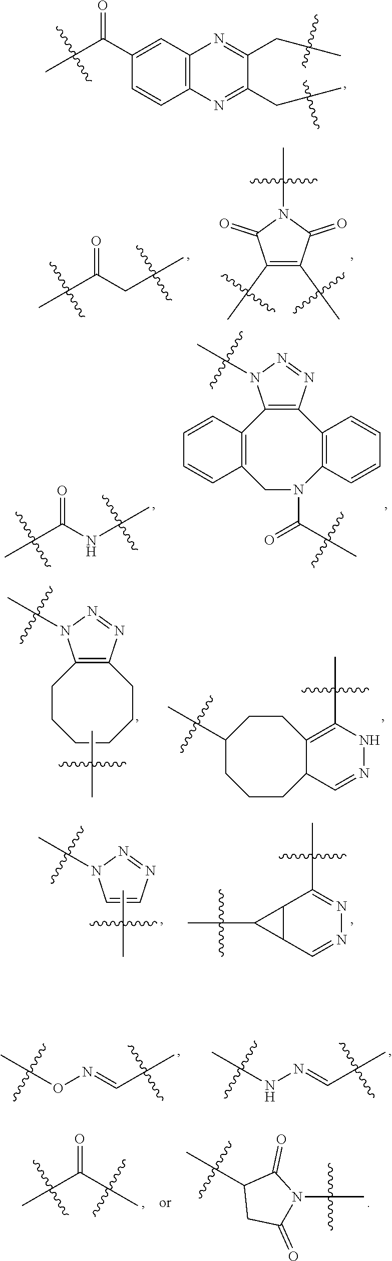

In embodiments, L¹ is

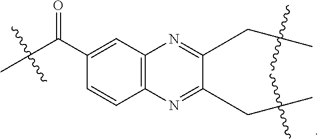 [SMILES: *Cc1nc2ccc(C(*)=O)cc2nc1C*]

In embodiments, L¹ is

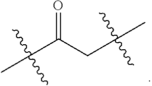 [SMILES: *CC(*)=O]

In embodiments, L¹ is

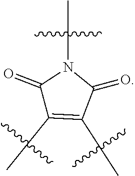 [SMILES: *C1=C(*)C(=O)N(*)C1=O]

In embodiments, L¹ is

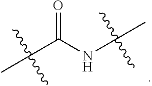 [SMILES: *NC(*)=O]

In embodiments, L¹ is

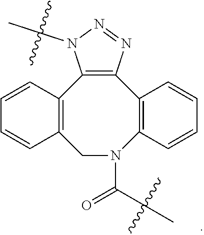 [SMILES: *C(=O)N1Cc2ccccc2-c2c(nnn2*)-c2ccccc21]

In embodiments, L¹ is

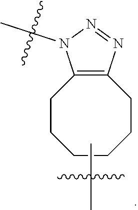

In embodiments, L¹ is

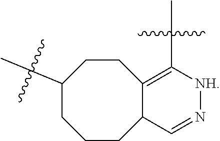 [SMILES: *C1=C2CCC(*)CCCC2C=NN1]

In embodiments, L¹ is

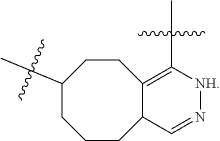 [SMILES: *C1=C2CCC(*)CCCC2C=NN1]

In embodiments, L¹ is

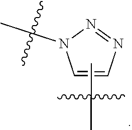

In embodiments, L¹ is

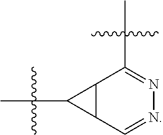 [SMILES: *C1=NN=CC2C(*)C12]

In embodiments, L¹ is

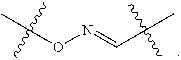 [SMILES: */C=N/O*]

In embodiments, L¹ is

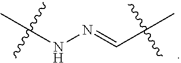 [SMILES: */C=N/N*]

In embodiments, L¹ is

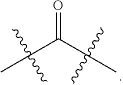 [SMILES: *C(*)=O]

In embodiments, L is

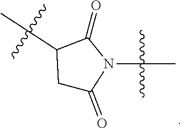 [SMILES: *C1CC(=O)N(*)C1=O]

Where L¹ is

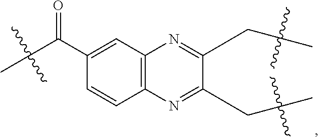 [SMILES: *Cc1nc2ccc(C(*)=O)cc2nc1C*]

the two CH₂ moieties shown on the right side of the structure may each be bound to a different cysteine of the anti-HER2 antibody via a thiol group. Where L¹ is

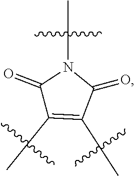 [SMILES: *C1=C(*)C(=O)N(*)C1=O]

the two alkene carbons shown on the bottom of the structure may each be bound to a different cysteine of the anti-HER2 antibody via a thiol group. Where L¹ is

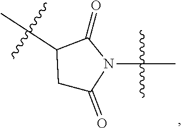 [SMILES: *C1CC(=O)N(*)C1=O]

the carbon may be bound to a cysteine of the anti-HER2 antibody via a thiol group.

In embodiments, L² is a bond, —C(O)—, —NH—, -Val-, -Phe-, -Lys-, -Gly-, -(4-aminobenzyloxycarbonyl)-, —(C(O)N(R²)CH₂CH₂N(R³))—, -Ser-, -Thr-, -Ala-, -β-Ala-, -citrulline- (Cit), —(CH₂)ₙ—, —(CH₂CH₂O)ₙ—, or any combination thereof.

In embodiments, each R² and R³ is independently H or substituted or unsubstituted alkyl (e.g., C₁-C₈ alkyl, C₁-C₆ alkyl, or C₁-C₄ alkyl). In embodiments, each R² and R³ is independently H. In embodiments, each R2 and R3 is independently substituted or unsubstituted alkyl. In embodiments, each R² and R³ is independently substituted or unsubstituted alkyl (e.g., C₁-C₈ alkyl, C₁-C₆ alkyl, or C₁-C₄ alkyl). In embodiments, each R2 and R3 is independently unsubstituted alkyl (e.g., C₁-C₈ alkyl, C₁-C₆ alkyl, or C₁-C₄ alkyl). In embodiments, each R2 and R³ is independently substituted alkyl (e.g., C₁-C₈ alkyl, C₁-C₆ alkyl, or C₁-C₄ alkyl).

In embodiments, each R² and R³ is independently H or substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, each R² and R³ is independently substituted (e.g., substituted with at least one substituent group, size-limited substituent group, or lower substituent group) or unsubstituted alkyl. In embodiments, each R² and R³ is independently substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, each R² and R³ is independently unsubstituted alkyl (e.g., C₁-C₈ alkyl, C₁-C₆ alkyl, or C₁-C₄ alkyl). In embodiments, each R² and R³ is independently substituted (e.g., substituted with at least one substituent group, size-limited substituent group, or lower substituent group) alkyl (e.g., C₁-C₈ alkyl, C₁-C₆ alkyl, or C₁-C₄ alkyl).

In embodiments, each R² and R³ is independently methyl, ethyl, propyl, isopropyl, butyl, isobutyl, tert-butyl, pentyl, isopentyl, or hexyl. In embodiments, each R² and R³ is independently methyl. In embodiments, each R² and R³ is independently ethyl. In embodiments, each R² and R³ is independently propyl. In embodiments, each R² and R³ is independently butyl.

In embodiments, L² is a bond, —C(O)—, —NH—, -Val-, -Phe-, -Lys-, -Gly-, -(4-aminobenzyloxycarbonyl)-, —(C(O)N(CH₃)CH₂CH₂N(CH₃))—, -Ser-, -Thr-, -Ala-, -β-Ala-, —O—, -citrulline- (Cit), —(CH₂)ₙ—, —(CH₂CH₂O)ₙ—, or any combination thereof.

In embodiments, L² is —C(O)—, —NH—, -Val-, -Gly-, -Cit-, -Ala-, —O—, -(4-aminobenzyloxycarbonyl)-, —(CH₂)ₙ—, —(CH₂CH₂O)ₙ—, —(C(O)N(CH₃)CH₂CH₂N(CH₃))—, or any combination thereof.

In embodiments, L² is —C(O)—, —NH—, -Gly-, —(CH₂)ₙ—, —(CH₂CH₂O)ₙ—, or any combination thereof.

In embodiments, L² is —C(O)—, —NH—, -Val-, -Cit-, —(CH₂CH₂O)ₙ—, -(4-aminobenzyloxycarbonyl)-, —(CH₂)ₙ—, —(C(O)N(CH₃)CH₂CH₂N(CH₃))—, or any combination thereof.

In embodiments, L² is:

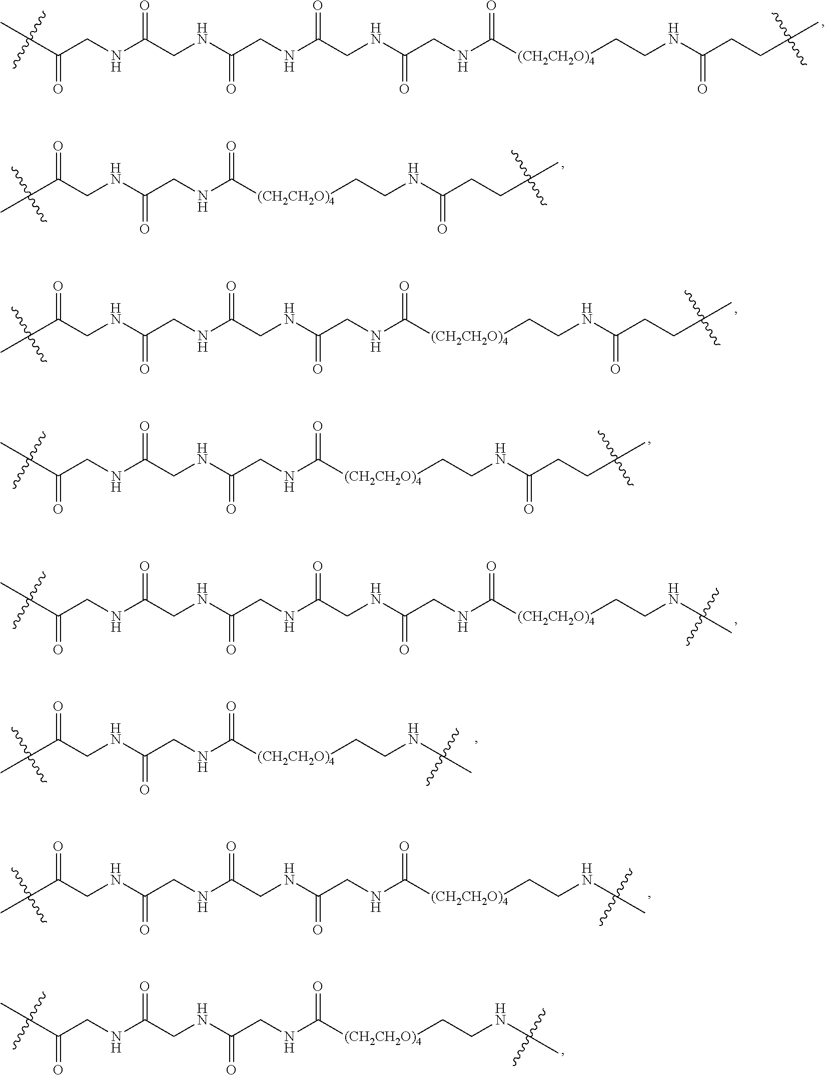

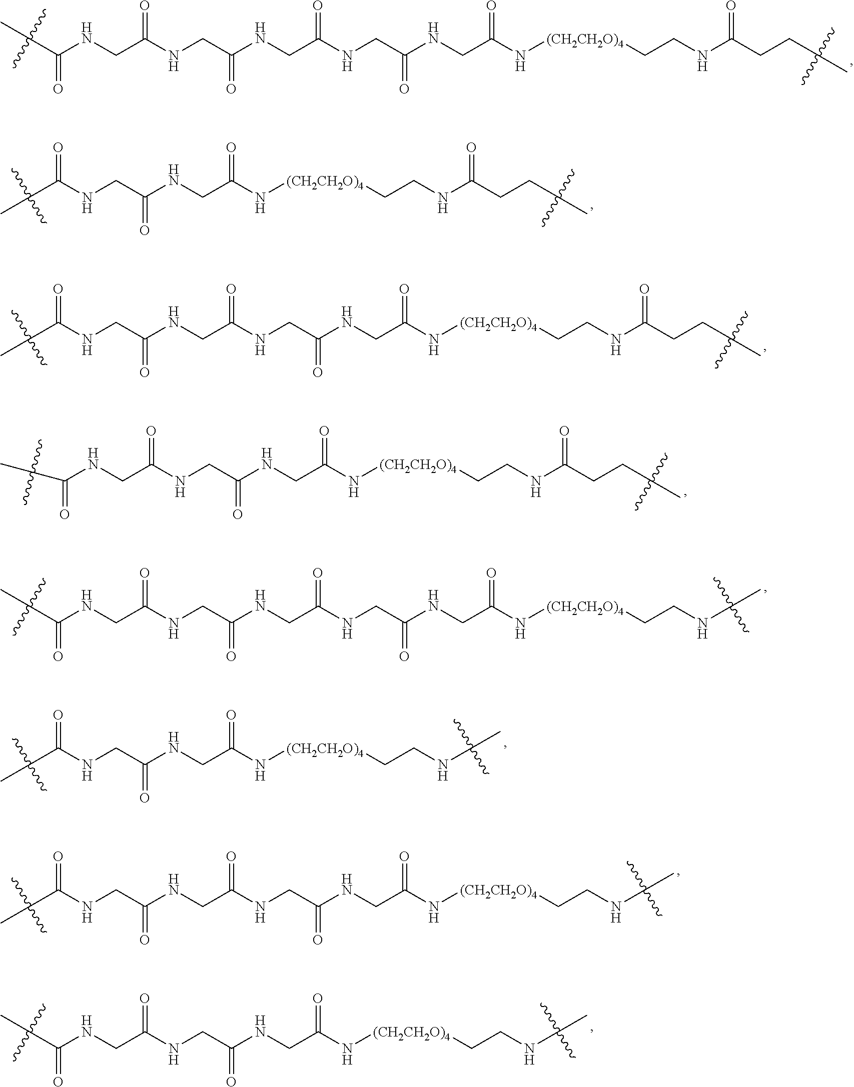

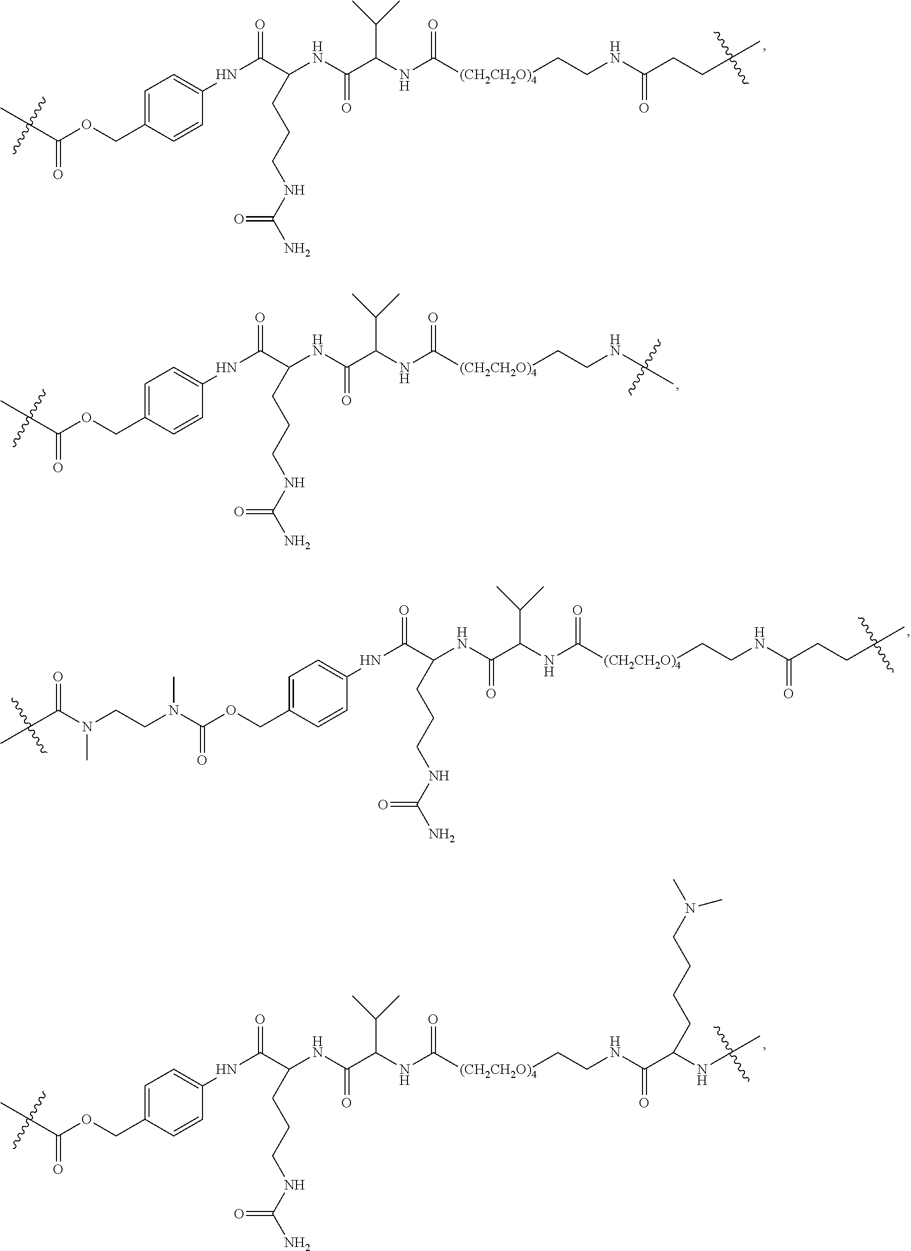

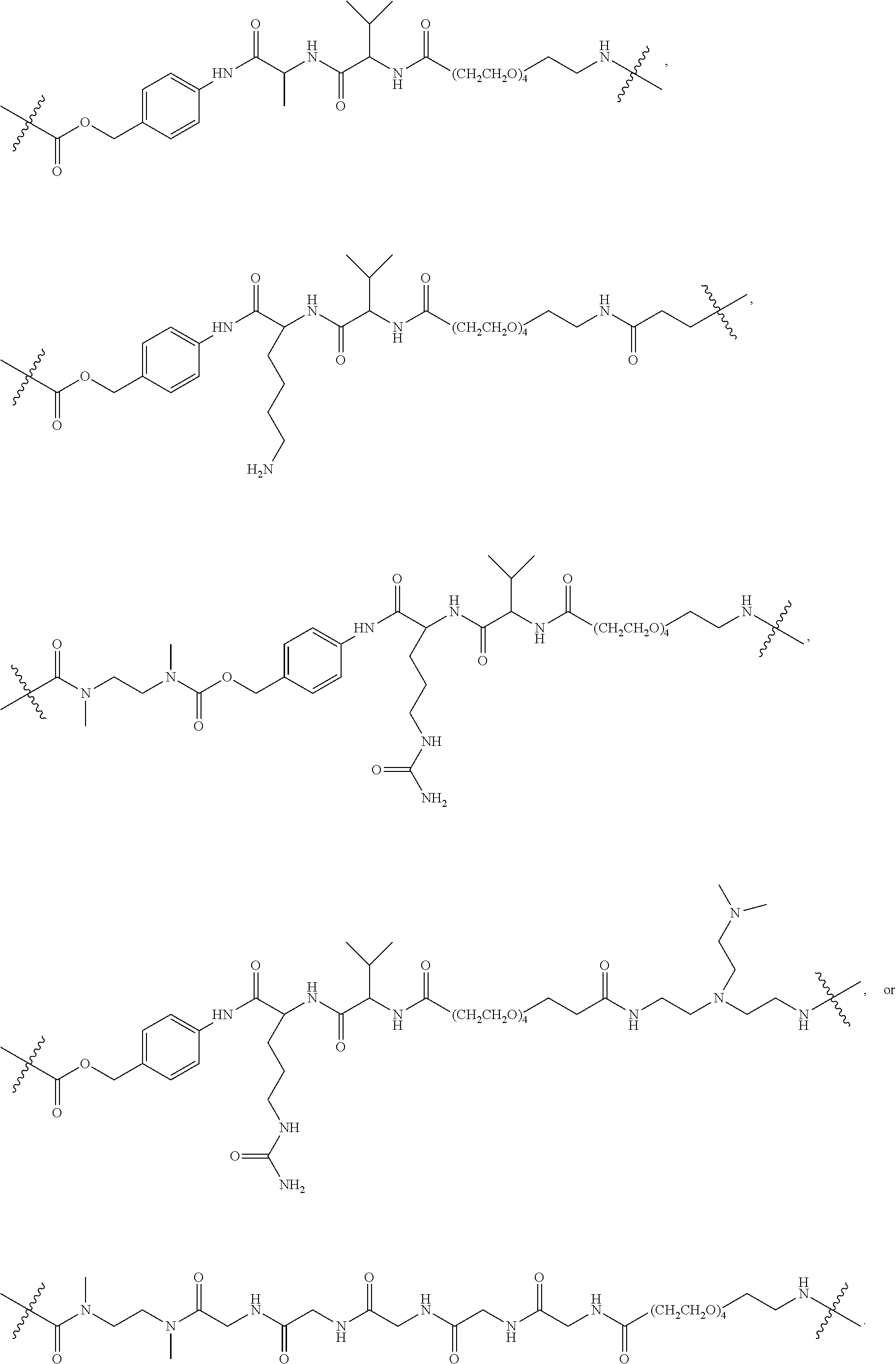

In embodiments, L² is

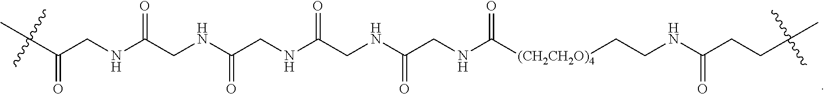

In embodiments, L² is

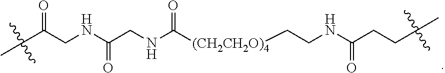

In embodiments L² is

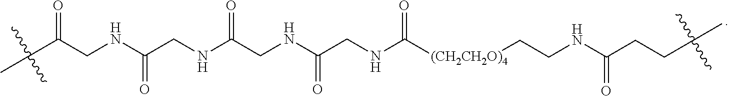

In embodiments, L² is

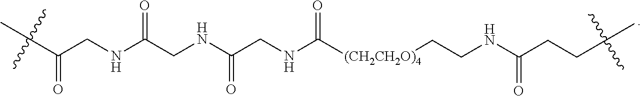

In embodiments, L² is

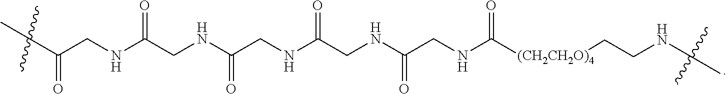

In embodiments, L² is

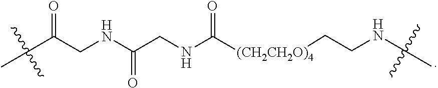

In embodiments, L² is

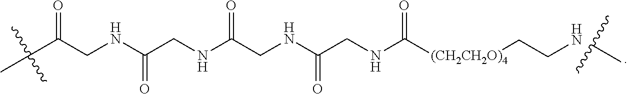

In embodiments, L² is

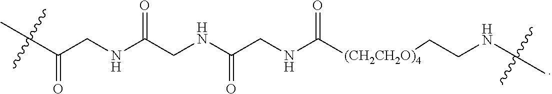

In embodiments, L² is

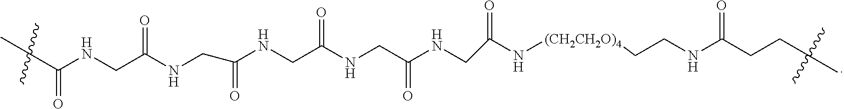

In embodiments, L² is

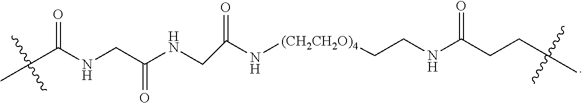

In embodiments, L² is

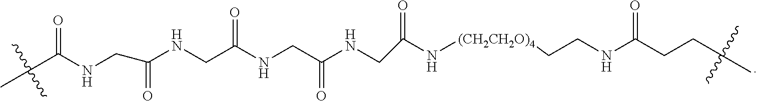

In embodiments, L² is

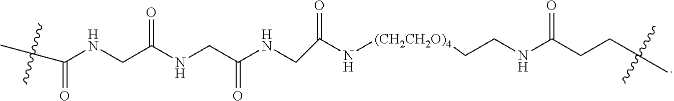

In embodiments, L² is

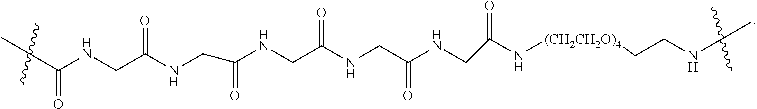

In embodiments, L² is

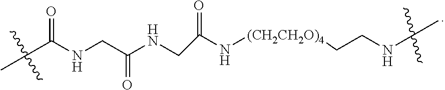

In embodiments, L² is

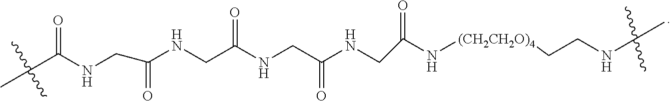

In embodiments, L² is

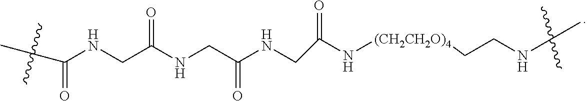

In embodiments, L² is

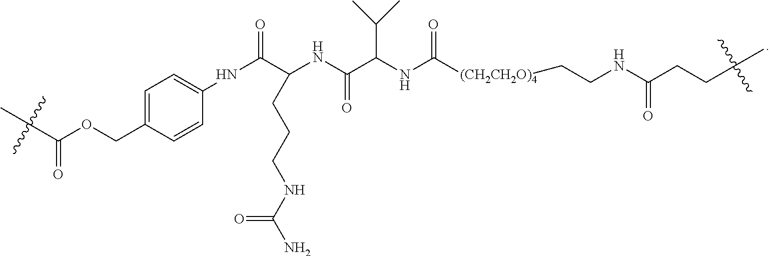

In embodiments, L² is

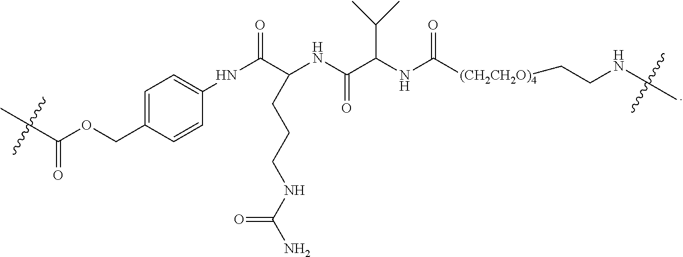

In embodiments, L² is

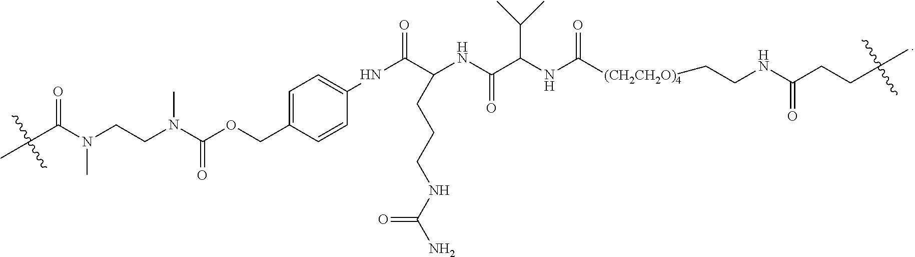

In embodiments, L² is

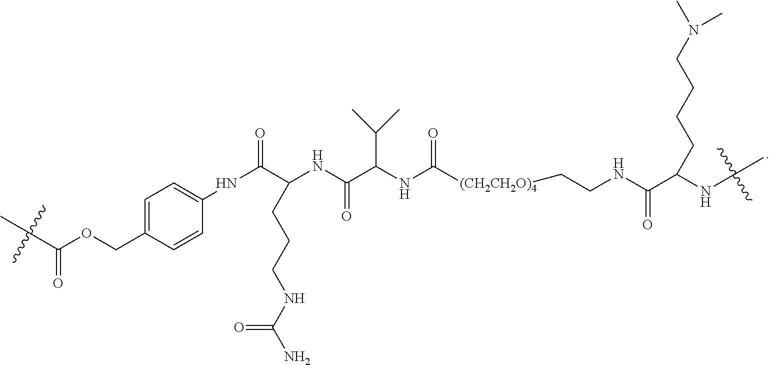

In embodiments, L² is

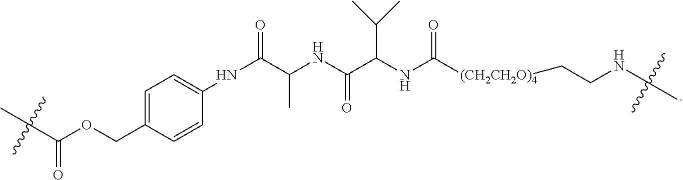

In embodiments, L² is

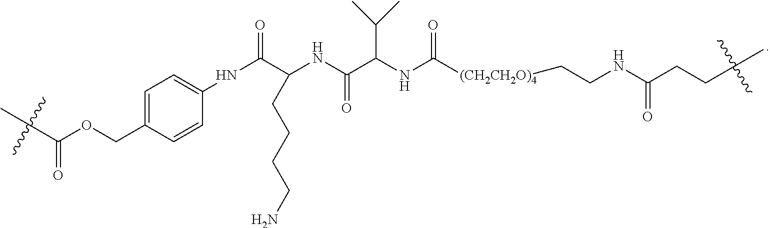

In embodiments, L² is

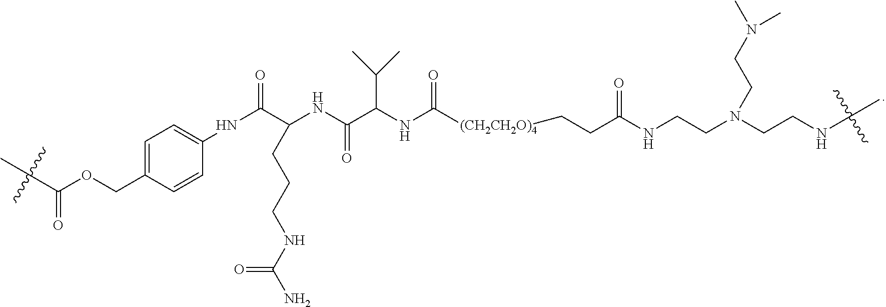

In embodiments, L² is

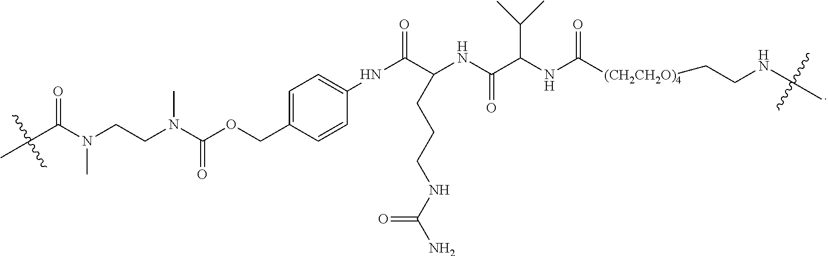

In embodiments, L² is

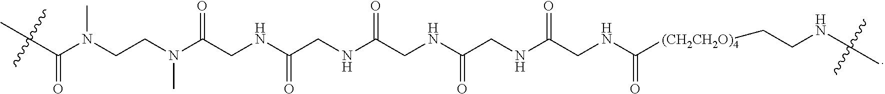

In embodiments, L² is a bond. In embodiments, L² is —C(O)—. In embodiments, L² is —NH—. In embodiments, L² is -Val-. In embodiments, L² is -Phe-. In embodiments, L² is -Lys-. In embodiments, L² is -(4-aminobenzyloxycarbonyl)-. In embodiments, L² is —(CH₂)ₙ. In embodiments, L² is —(CH₂CH₂O)ₙ—. In embodiments, L² is -Gly-. In embodiments, L² is -Ser-. In embodiments, L² is -Thr-. In embodiments, L² is -Ala-. In embodiments, L² is -Q-Ala-. In embodiments, L² is -Cit-. In embodiments, L² is —O—.

In embodiments, -L¹-L²- is

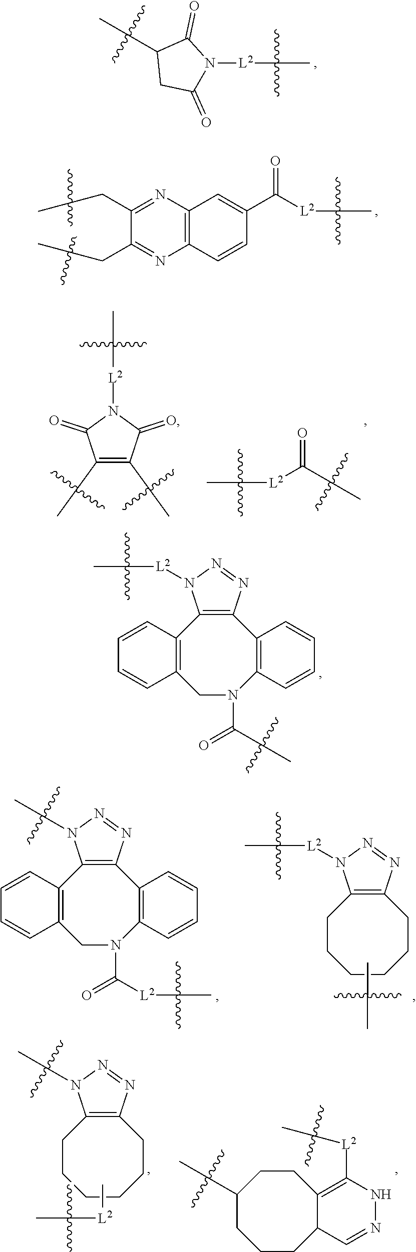

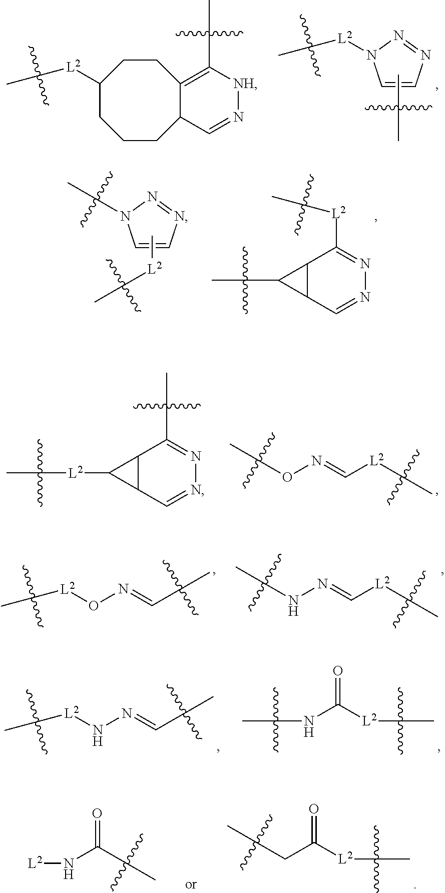

In embodiments, -L¹-L²- is

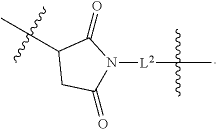

In embodiments, -L¹-L²- is

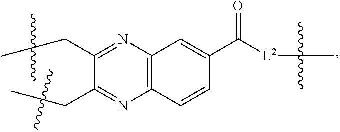

where the two CH₂ moieties shown on the left side of the structure may each be bound to a separate sulfur of the monoclonal antibody. In embodiments, -L¹-L²- is

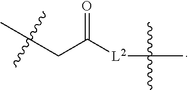

In embodiments, -L¹-L²- is

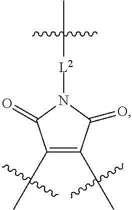

where the two alkene carbons shown on the bottom of the structure may each be bound to a separate sulfur of the monoclonal antibody. In embodiments, -L¹-L²- is

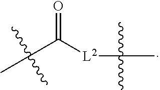

In embodiments, -L¹-L²- is

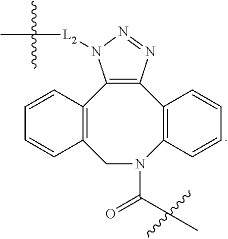

In embodiments, -L¹-L²- is

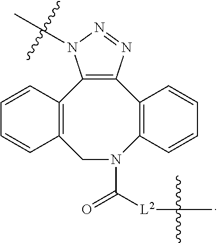

In embodiments, -L¹-L²- is

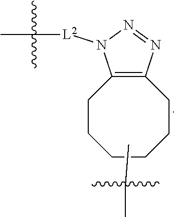

In embodiments, -L¹-L²- is

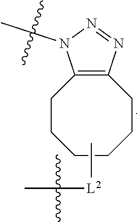

In embodiments, -L¹-L²- is

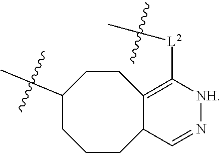

In embodiments, -L¹-L²- is

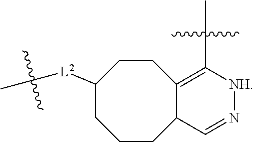

In embodiments, -L¹-L²- is

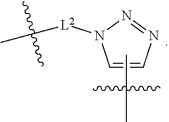

In embodiments, -L¹-L²- is

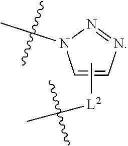

In embodiments, -L¹-L²- is

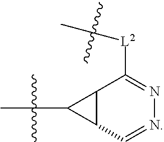

In embodiments, -L¹-L²- is

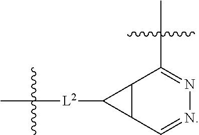

In embodiments, -L¹-L²- is

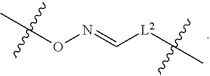

In embodiments, -L¹-L²- is

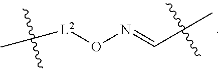

In embodiments, -L¹-L²- is

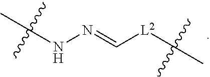

In embodiments, -L¹-L²- is

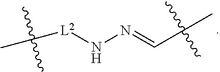

In embodiments, -L¹-L²- is

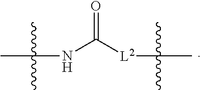

In embodiments, -L¹-L²- is

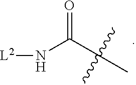

In embodiments, L³ is substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene), substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted —OCH₂-(heterocycloalkyl (e.g., 3 to 8 membered heterocycloalkyl, 3 to 6 membered heterocycloalkyl, or 5 to 6 membered heterocycloalkyl)), substituted (e.g., with a substituent group, a size-limited substituent group or a lower substituent group) or unsubstituted —OCH₂-(heteroaryl (e.g., 5 to 10 membered heteroaryl, 5 to 9 membered heteroaryl, or 5 to 6 membered heteroaryl)), substituted (e.g. with a substituent group, a size-limited substituent group or a lower substituent group) or unsubstituted —CH₂NCH₂-(heterocycloalkyl (e.g., 3 to 8 membered heterocycloalkyl, 3 to 6 membered heterocycloalkyl, or 5 to 6 membered heterocycloalkyl)), or substituted (e.g., with a substituent group, a size-limited substituent group or a lower substituent group) or unsubstituted —CH₂NCH₂-(heteroaryl (e.g., 5 to 10 membered heteroaryl, 5 to 9 membered heteroaryl, or 5 to 6 membered heteroaryl)). In embodiments, L³ is substituted with one or more substituent groups. In embodiments, L³ is substituted with one or more size-limited substituent groups. In embodiments, L³ is substituted with one or more lower substituent groups.

In embodiments, L³ is substituted (e.g., with a substituent group, a size-limited substituent group or a lower substituent group) heterocycloalkylene (e.g., 3 to 8 membered heterocycloalkylene, 3 to 6 membered heterocycloalkylene, or 5 to 6 membered heterocycloalkylene). In embodiments, L³ is unsubstituted heterocycloalkylene (e.g., 3 to 8 membered heterocycloalkylene, 3 to 6 membered heterocycloalkylene, or 5 to 6 membered heterocycloalkylene). In embodiments, L³ is substituted (e.g., with a substituent group, a size-limited substituent group or a lower substituent group) heteroarylene (e.g., 5 to 10 membered heteroarylene, 5 to 9 membered heteroarylene, or 5 to 6 membered heteroarylene). In embodiments, L³ is unsubstituted heteroarylene (e.g., 5 to 10 membered heteroarylene, 5 to 9 membered heteroarylene, or 5 to 6 membered heteroarylene). In embodiments, L³ is substituted (e.g., with a substituent group, a size-limited substituent group, or a lower substituent group) heterocycloalkyl (e.g., 3 to 8 membered heterocycloalkyl, 3 to 6 membered heterocycloalkyl, or 5 to 6 membered heterocycloalkyl). In embodiments, L³ is unsubstituted heterocycloalkyl (e.g., 3 to 8 membered heterocycloalkyl, 3 to 6 membered heterocycloalkyl, or 5 to 6 membered heterocycloalkyl). In embodiments, L³ is substituted (e.g., with a substituent group, a size-limited substituent group, or a lower substituent group) heteroaryl (e.g., 5 to 10 membered heteroaryl, 5 to 9 membered heteroaryl, or 5 to 6 membered heteroaryl). In embodiments, L³ is unsubstituted heteroaryl (e.g., 5 to 10 membered heteroaryl, 5 to 9 membered heteroaryl, or 5 to 6 membered heteroaryl). In embodiments, L³ is substituted (e.g., with a substituent group, a size-limited substituent group, or a lower substituent group) —OCH₂-(heterocycloalkyl (e.g., 3 to 8 membered heterocycloalkyl, 3 to 6 membered heterocycloalkyl, or 5 to 6 membered heterocycloalkyl)). In embodiments, L³ is unsubstituted —OCH₂-(heterocycloalkyl (e.g., 3 to 8 membered heterocycloalkyl, 3 to 6 membered heterocycloalkyl, or 5 to 6 membered heterocycloalkyl)). In embodiments, L³ is substituted (e.g., with a substituent group, a size-limited substituent group, or a lower substituent group) —OCH₂-(heteroaryl (e.g., 5 to 10 membered heteroaryl, 5 to 9 membered heteroaryl, or 5 to 6 membered heteroaryl)). In embodiments, L³ is unsubstituted —OCH₂-(heteroaryl (e.g., 5 to 10 membered heteroaryl, 5 to 9 membered heteroaryl, or 5 to 6 membered heteroaryl)). In embodiments, L³ is substituted (e.g., with a substituent group, a size-limited substituent group, or a lower substituent group) —CH₂NCH₂-(heterocycloalkyl (e.g., 3 to 8 membered heterocycloalkyl, 3 to 6 membered heterocycloalkyl, or 5 to 6 membered heterocycloalkyl)). In embodiments, L³ is unsubstituted —CH₂NCH₂-(heterocycloalkyl (e.g., 3 to 8 membered heterocycloalkyl, 3 to 6 membered heterocycloalkyl, or 5 to 6 membered heterocycloalkyl)). In embodiments, L³ is substituted (e.g., with a substituent group, a size-limited substituent group, or a lower substituent group) —CH₂NCH₂-(heteroaryl (e.g., 5 to 10 membered heteroaryl, 5 to 9 membered heteroaryl, or 5 to 6 membered heteroaryl)). In embodiments, L³ is unsubstituted —CH₂NCH₂-(heteroaryl (e.g., 5 to 10 membered heteroaryl, 5 to 9 membered heteroaryl, or 5 to 6 membered heteroaryl)).

In embodiments, L³ is substituted (e.g., with a substituent group, a size-limited substituent group or a lower substituent group) or unsubstituted 3 to 8 membered heterocycloalkylene. In embodiments, L³ is substituted (e.g., with a substituent group, a size-limited substituent group or a lower substituent group) 3 to 8 membered heterocycloalkylene. In embodiments, L³ is unsubstituted 3 to 8 membered heterocycloalkylene. In embodiments, L³ is substituted (e.g., with a substituent group, a size-limited substituent group, or a lower substituent group) or unsubstituted 3 to 8 membered heterocycloalkyl. In embodiments, L³ is substituted (e.g., with a substituent group, a size-limited substituent group, or a lower substituent group) 3 to 8 membered heterocycloalkyl. In embodiments, L³ is unsubstituted 3 to 8 membered heterocycloalkyl. In embodiments, L³ is substituted (e.g., with a substituent group, a size-limited substituent group, or a lower substituent group) or unsubstituted —CH₂NCH₂-(3 to 8 membered heterocycloalkyl). In embodiments, L³ is substituted (e.g., with a substituent group, a size-limited substituent group, or a lower substituent group) —CH₂NCH₂-(3 to 8 membered heterocycloalkyl). In embodiments, L³ is unsubstituted —CH₂NCH₂-(3 to 8 membered heterocycloalkyl). In embodiments, L³ is substituted (e.g., with a substituent group, a size-limited substituent group, or a lower substituent group) or unsubstituted —OCH₂-(3 to 8 membered heterocycloalkyl). In embodiments, L³ is substituted (e.g., with a substituent group, a size-limited substituent group, or a lower substituent group) —OCH₂-(3 to 8 membered heterocycloalkyl). In embodiments, L³ is unsubstituted —OCH₂-(3 to 8 membered heterocycloalkyl).

In embodiments, L³ is substituted (e.g., with a substituent group, a size-limited substituent group or a lower substituent group) or unsubstituted 3 to 6 membered heterocycloalkylene. In embodiments, L³ is substituted (e.g., with a substituent group, a size-limited substituent group or a lower substituent group) 3 to 6 membered heterocycloalkylene. In embodiments, L³ is unsubstituted 3 to 6 membered heterocycloalkylene. In embodiments, L³ is substituted (e.g., with a substituent group, a size-limited substituent group, or a lower substituent group) or unsubstituted 3 to 6 membered heterocycloalkyl. In embodiments, L³ is substituted (e.g., with a substituent group, a size-limited substituent group, or a lower substituent group) 3 to 6 membered heterocycloalkyl. In embodiments, L³ is unsubstituted 3 to 6 membered heterocycloalkyl. In embodiments, L³ is substituted (e.g., with a substituent group, a size-limited substituent group, or a lower substituent group) or unsubstituted —CH₂NCH₂-(3 to 6 membered heterocycloalkyl). In embodiments, L³ is substituted (e.g., with a substituent group, a size-limited substituent group, or a lower substituent group) —CH₂NCH₂-(3 to 6 membered heterocycloalkyl). In embodiments, L³ is unsubstituted —CH₂NCH₂-(3 to 6 membered heterocycloalkyl). In embodiments, L³ is substituted (e.g., with a substituent group, a size-limited substituent group, or a lower substituent group) or unsubstituted —OCH₂-(3 to 6 membered heterocycloalkyl). In embodiments, L³ is substituted (e.g., with a substituent group, a size-limited substituent group, or a lower substituent group) —OCH₂-(3 to 6 membered heterocycloalkyl). In embodiments, L³ is unsubstituted —OCH₂-(3 to 6 membered heterocycloalkyl).

In embodiments, L³ is substituted (e.g., with a substituent group, a size-limited substituent group or a lower substituent group) or unsubstituted heterocyclobutylene, heterocyclopentylene or heterocyclohexylene. In embodiments, L³ is substituted (e.g., with a substituent group, a size-limited substituent group or a lower substituent group) heterocyclobutylene, heterocyclopentylene or heterocyclohexylene. In embodiments, L³ is unsubstituted heterocyclobutylene, heterocyclopentylene or heterocyclohexylene. In embodiments, L³ is substituted (e.g., with a substituent group, a size-limited substituent group or a lower substituent group) or unsubstituted heterocyclobutyl, heterocyclopentyl or heterocyclohexyl. In embodiments, L³ is substituted (e.g., with a substituent group, a size-limited substituent group, or a lower substituent group) heterocyclobutyl, heterocyclopentyl or heterocyclohexyl. In embodiments, L³ is unsubstituted heterocyclobutyl, heterocyclopentyl or heterocyclohexyl. In embodiments, L³ is substituted (e.g., with a substituent group, a size-limited substituent group or a lower substituent group) or unsubstituted —CH₂NCH₂-(heterocyclobutyl, heterocyclopentyl, or heterocyclohexyl). In embodiments, L³ is substituted (e.g., with a substituent group, a size-limited substituent group or a lower substituent group) —CH₂NCH₂-(heterocyclobutyl, heterocyclopentyl, or heterocyclohexyl). In embodiments, L³ is unsubstituted —CH₂NCH₂-(heterocyclobutyl, heterocyclopentyl, or heterocyclohexyl). In embodiments, L³ is substituted (e.g., with a substituent group, a size-limited substituent group or a lower substituent group) or unsubstituted —OCH₂-(heterocyclobutyl, heterocyclopentyl, or heterocyclohexyl). In embodiments, L³ is substituted (e.g., with a substituent group, a size-limited substituent group or a lower substituent group) —OCH₂-(heterocyclobutyl, heterocyclopentyl, or heterocyclohexyl). In embodiments, L³ is unsubstituted —OCH₂-(heterocyclobutyl, heterocyclopentyl, or heterocyclohexyl).

In embodiments, L³ is substituted (e.g., with a substituent group, a size-limited substituent group or a lower substituent group) or unsubstituted heterocyclobutylene. In embodiments, L³ is substituted (e.g., with a substituent group, a size-limited substituent group or a lower substituent group) heterocyclobutylene. In embodiments, L³ is unsubstituted heterocyclobutylene. In embodiments, L³ is substituted (e.g., with a substituent group, a size-limited substituent group or a lower substituent group) or unsubstituted heterocyclobutyl. In embodiments, L³ is substituted (e.g., with a substituent group, a size-limited substituent group or a lower substituent group) heterocyclobutyl. In embodiments, L³ is unsubstituted heterocyclobutyl. In embodiments, L³ is substituted (e.g., with a substituent group, a size-limited substituent group or a lower substituent group) or unsubstituted —CH₂NCH₂-(heterocyclobutyl). In embodiments, L³ is substituted (e.g., with a substituent group, a size-limited substituent group or a lower substituent group) —CH₂NCH₂-(heterocyclobutyl). In embodiments, L³ is unsubstituted —CH₂NCH₂-(heterocyclobutyl). In embodiments, L³ is substituted (e.g., with a substituent group, a size-limited substituent group or a lower substituent group) or unsubstituted —OCH₂-(heterocyclobutyl). In embodiments, L³ is substituted (e.g., with a substituent group, a size-limited substituent group or a lower substituent group) —OCH₂-(heterocyclobutyl). In embodiments, L³ is unsubstituted —OCH₂-(heterocyclobutyl).

In embodiments, L³ is substituted (e.g., with a substituent group, a size-limited substituent group or a lower substituent group) or unsubstituted heterocyclopentylene. In embodiments, L³ is substituted (e.g., with a substituent group, a size-limited substituent group or a lower substituent group) heterocyclopentylene. In embodiments, L³ is unsubstituted heterocyclopentylene. In embodiments, L³ is substituted (e.g., with a substituent group, a size-limited substituent group or a lower substituent group) or unsubstituted heterocyclopentyl. In embodiments, L³ is substituted (e.g., with a substituent group, a size-limited substituent group or a lower substituent group) heterocyclopentyl. In embodiments, L³ is unsubstituted heterocyclopentyl. In embodiments, L³ is substituted (e.g., with a substituent group, a size-limited substituent group or a lower substituent group) or unsubstituted —CH₂NCH₂-(heterocyclopentyl). In embodiments, L³ is substituted (e.g., with a substituent group, a size-limited substituent group or a lower substituent group) —CH₂NCH₂-(heterocyclopentyl). In embodiments, L³ is unsubstituted —CH₂NCH₂-(heterocyclopentyl). In embodiments, L³ is substituted (e.g., with a substituent group, a size-limited substituent group or a lower substituent group) or unsubstituted —OCH₂-(heterocyclopentyl). In embodiments, L³ is substituted (e.g., with a substituent group, a size-limited substituent group or a lower substituent group) —OCH₂-(heterocyclopentyl). In embodiments, L³ is unsubstituted —OCH₂-(heterocyclopentyl).

In embodiments, L³ is substituted (e.g., with a substituent group, a size-limited substituent group or a lower substituent group) or unsubstituted heterocyclohexylene. In embodiments, L³ is substituted (e.g., with a substituent group, a size-limited substituent group or a lower substituent group) heterocyclohexylene. In embodiments, L³ is unsubstituted heterocyclohexylene. In embodiments, L³ is substituted (e.g., with a substituent group, a size-limited substituent group or a lower substituent group) or unsubstituted heterocyclohexyl. In embodiments, L³ is substituted (e.g., with a substituent group, a size-limited substituent group or a lower substituent group) heterocyclohexyl. In embodiments, L³ is unsubstituted heterocyclohexyl. In embodiments, L³ is substituted (e.g., with a substituent group, a size-limited substituent group or a lower substituent group) or unsubstituted —CH₂NCH₂-(heterocyclohexyl). In embodiments, L³ is substituted (e.g., with a substituent group, a size-limited substituent group or a lower substituent group) —CH₂NCH₂-(heterocyclohexyl). In embodiments, L³ is unsubstituted —CH₂NCH₂-(heterocyclohexyl). In embodiments, L³ is substituted (e.g., with a substituent group, a size-limited substituent group or a lower substituent group) or unsubstituted —OCH₂-(heterocyclohexyl). In embodiments, L³ is substituted (e.g., with a substituent group, a size-limited substituent group or a lower substituent group) —OCH₂-(heterocyclohexyl). In embodiments, L³ is unsubstituted —OCH₂-(heterocyclohexyl).

In embodiments, L³ is substituted (e.g., with a substituent group, a size-limited substituent group or a lower substituent group) or unsubstituted 5 to 10 membered heteroarylene. In embodiments, L³ is substituted (e.g., with a substituent group, a size-limited substituent group or a lower substituent group) 5 to 10 membered heteroarylene. In embodiments, L³ is unsubstituted 5 to 10 membered heteroarylene. In embodiments, L³ is substituted (e.g., with a substituent group, a size-limited substituent group or a lower substituent group) or unsubstituted 5 to 10 membered heteroaryl. In embodiments, L³ is substituted (e.g., with a substituent group, a size-limited substituent group or a lower substituent group) 5 to 10 membered heteroaryl. In embodiments, L³ is unsubstituted 5 to 10 membered heteroaryl. In embodiments, L³ is substituted (e.g., with a substituent group, a size-limited substituent group or a lower substituent group) or unsubstituted —CH₂NCH₂-(5 to 10 membered heteroaryl). In embodiments, L³ is substituted (e.g., with a substituent group, a size-limited substituent group or a lower substituent group) —CH₂NCH₂-(5 to 10 membered heteroaryl). In embodiments, L³ is unsubstituted —CH₂NCH₂-(5 to 10 membered heteroaryl). In embodiments, L³ is substituted (e.g., with a substituent group, a size-limited substituent group or a lower substituent group) or unsubstituted —OCH₂-(5 to 10 membered heteroaryl). In embodiments, L³ is substituted (e.g., with a substituent group, a size-limited substituent group or a lower substituent group) —OCH₂-(5 to 10 membered heteroaryl). In embodiments, L³ is unsubstituted —OCH₂-(5 to 10 membered heteroaryl).

In embodiments, L³ is substituted (e.g., with a substituent group, a size-limited substituent group or a lower substituent group) or unsubstituted 5 to 9 membered heteroarylene. In embodiments, L³ is substituted (e.g., with a substituent group, a size-limited substituent group or a lower substituent group) 5 to 9 membered heteroarylene. In embodiments, L³ is unsubstituted 5 to 9 membered heteroarylene. In embodiments, L³ is substituted (e.g., with a substituent group, a size-limited substituent group or a lower substituent group) or unsubstituted 5 to 9 membered heteroaryl. In embodiments, L³ is substituted (e.g., with a substituent group, a size-limited substituent group or a lower substituent group) 5 to 9 membered heteroaryl. In embodiments, L³ is unsubstituted 5 to 9 membered heteroaryl. In embodiments, L³ is substituted (e.g., with a substituent group, a size-limited substituent group or a lower substituent group) or unsubstituted —CH₂NCH₂-(5 to 9 membered heteroaryl). In embodiments, L³ is substituted (e.g., with a substituent group, a size-limited substituent group or a lower substituent group) —CH₂NCH₂-(5 to 9 membered heteroaryl). In embodiments, L³ is unsubstituted —CH₂NCH₂-(5 to 9 membered heteroaryl). In embodiments, L³ is substituted (e.g., with a substituent group, a size-limited substituent group or a lower substituent group) or unsubstituted —OCH₂-(5 to 9 membered heteroaryl). In embodiments, L³ is substituted (e.g., with a substituent group, a size-limited substituent group or a lower substituent group) —OCH₂-(5 to 9 membered heteroaryl). In embodiments, L³ is unsubstituted —OCH₂-(5 to 9 membered heteroaryl).

In embodiments, L³ is substituted (e.g., with a substituent group, a size-limited substituent group or a lower substituent group) or unsubstituted 5 to 6 membered heteroarylene. In embodiments, L³ is substituted (e.g., with a substituent group, a size-limited substituent group or a lower substituent group) 5 to 6 membered heteroarylene. In embodiments, L³ is unsubstituted 5 to 6 membered heteroarylene. In embodiments, L³ is substituted (e.g., with a substituent group, a size-limited substituent group or a lower substituent group) or unsubstituted 5 to 6 membered heteroaryl. In embodiments, L³ is substituted (e.g., with a substituent group, a size-limited substituent group or a lower substituent group) 5 to 6 membered heteroaryl. In embodiments, L³ is unsubstituted 5 to 6 membered heteroaryl. In embodiments, L³ is substituted (e.g., with a substituent group, a size-limited substituent group or a lower substituent group) or unsubstituted —CH₂NCH₂-(5 to 6 membered heteroaryl). In embodiments, L³ is substituted (e.g., with a substituent group, a size-limited substituent group or a lower substituent group) —CH₂NCH₂-(5 to 6 membered heteroaryl). In embodiments, L³ is unsubstituted —CH₂NCH₂-(5 to 6 membered heteroaryl). In embodiments, L³ is substituted (e.g., with a substituent group, a size-limited substituent group or a lower substituent group) or unsubstituted —OCH₂-(5 to 6 membered heteroaryl). In embodiments, L³ is substituted (e.g., with a substituent group, a size-limited substituent group or a lower substituent group) —OCH₂-(5 to 6 membered heteroaryl). In embodiments, L³ is unsubstituted —OCH₂-(5 to 6 membered heteroaryl).

In embodiments, L³ is substituted (e.g., with a substituent group, a size-limited substituent group or a lower substituent group) or unsubstituted furanylene, pyrrolylene, pyridylene, pyranylene, imidazolylene, thienylene, oxazolylene, or thiazolylene. In embodiments, L³ is substituted (e.g., with a substituent group, a size-limited substituent group or a lower substituent group) furanylene, pyrrolylene, pyridylene, pyranylene, imidazolylene, thienylene, oxazolylene, or thiazolylene. In embodiments, L³ is unsubstituted furanylene, pyrrolylene, pyridylene, pyranylene, imidazolylene, thienylene, oxazolylene, or thiazolylene. In embodiments, L³ is substituted (e.g., with a substituent group, a size-limited substituent group or a lower substituent group) or unsubstituted furanyl, pyrrolyl, pyridyl, pyranyl, imidazolyl, thienyl, oxazolyl, or thiazolyl. In embodiments, L³ is substituted (e.g., with a substituent group, a size-limited substituent group or a lower substituent group) furanyl, pyrrolyl, pyridyl, pyranyl, imidazolyl, thienyl, oxazolyl, or thiazolyl. In embodiments, L³ is unsubstituted furanyl, pyrrolyl, pyridyl, pyranyl, imidazolyl, thienyl, oxazolyl, or thiazolyl. In embodiments, L³ is substituted (e.g., with a substituent group, a size-limited substituent group or a lower substituent group) or unsubstituted —CH₂NCH₂-(furanyl, pyrrolyl, pyridyl, pyranyl, imidazolyl, thienyl, oxazolyl, or thiazolyl). In embodiments, L³ is substituted (e.g., with a substituent group, a size-limited substituent group or a lower substituent group) —CH₂NCH₂-(furanyl, pyrrolyl, pyridyl, pyranyl, imidazolyl, thienyl, oxazolyl, or thiazolyl). In embodiments, L³ is unsubstituted —CH₂NCH₂-(furanyl, pyrrolyl, pyridyl, pyranyl, imidazolyl, thienyl, oxazolyl, or thiazolyl). In embodiments, L³ is substituted (e.g., with a substituent group, a size-limited substituent group or a lower substituent group) or unsubstituted —OCH₂-(furanyl, pyrrolyl, pyridyl, pyranyl, imidazolyl, thienyl, oxazolyl, or thiazolyl). In embodiments, L³ is substituted (e.g., with a substituent group, a size-limited substituent group or a lower substituent group) —OCH₂-(furanyl, pyrrolyl, pyridyl, pyranyl, imidazolyl, thienyl, oxazolyl, or thiazolyl). In embodiments, L³ is unsubstituted —OCH₂-(furanyl, pyrrolyl, pyridyl, pyranyl, imidazolyl, thienyl, oxazolyl, or thiazolyl).

In embodiments, L³ is substituted (e.g., with a substituent group, a size-limited substituent group or a lower substituent group) or unsubstituted furanylene. In embodiments, L³ is substituted (e.g., with a substituent group, a size-limited substituent group or a lower substituent group) furanylene. In embodiments, L³ is unsubstituted furanylene. In embodiments, L³ is substituted (e.g., with a substituent group, a size-limited substituent group or a lower substituent group) or unsubstituted furanyl. In embodiments, L³ is substituted (e.g., with a substituent group, a size-limited substituent group or a lower substituent group) furanyl. In embodiments, L³ is unsubstituted furanyl. In embodiments, L³ is substituted (e.g., with a substituent group, a size-limited substituent group or a lower substituent group) or unsubstituted —CH₂NCH₂-(furanyl). In embodiments, L³ is substituted (e.g., with a substituent group, a size-limited substituent group or a lower substituent group) —CH₂NCH₂-(furanyl). In embodiments, L³ is unsubstituted —CH₂NCH₂-(furanyl). In embodiments, L³ is substituted (e.g., with a substituent group, a size-limited substituent group or a lower substituent group) or unsubstituted —OCH₂-(furanyl). In embodiments, L³ is substituted (e.g., with a substituent group, a size-limited substituent group or a lower substituent group) —OCH₂-(furanyl). In embodiments, L³ is unsubstituted —OCH₂-(furanyl).

In embodiments, L³ is substituted (e.g., with a substituent group, a size-limited substituent group or a lower substituent group) or unsubstituted pyrrolylene. In embodiments, L³ is substituted (e.g., with a substituent group, a size-limited substituent group or a lower substituent group) pyrrolylene. In embodiments, L³ is unsubstituted pyrrolylene. In embodiments, L³ is substituted (e.g., with a substituent group, a size-limited substituent group or a lower substituent group) or unsubstituted pyrrolyl. In embodiments, L³ is substituted (e.g., with a substituent group, a size-limited substituent group or a lower substituent group) pyrrolyl. In embodiments, L³ is unsubstituted pyrrolyl. In embodiments, L³ is substituted (e.g., with a substituent group, a size-limited substituent group or a lower substituent group) or unsubstituted —CH₂NCH₂-(pyrrolyl). In embodiments, L³ is substituted (e.g., with a substituent group, a size-limited substituent group or a lower substituent group) —CH₂NCH₂-(pyrrolyl). In embodiments, L³ is unsubstituted —CH₂NCH₂-(pyrrolyl). In embodiments, L³ is substituted (e.g., with a substituent group, a size-limited substituent group or a lower substituent group) or unsubstituted —OCH₂-(pyrrolyl). In embodiments, L³ is substituted (e.g., with a substituent group, a size-limited substituent group or a lower substituent group) —OCH₂-(pyrrolyl). In embodiments, L³ is unsubstituted —OCH₂-(pyrrolyl).

In embodiments, L³ is substituted (e.g., with a substituent group, a size-limited substituent group or a lower substituent group) or unsubstituted pyridylene. In embodiments, L³ is substituted (e.g., with a substituent group, a size-limited substituent group or a lower substituent group) pyridylene. In embodiments, L³ is unsubstituted pyridylene. In embodiments, L³ is substituted (e.g., with a substituent group, a size-limited substituent group or a lower substituent group) or unsubstituted pyridyl. In embodiments, L³ is substituted (e.g., with a substituent group, a size-limited substituent group or a lower substituent group) pyridyl. In embodiments, L³ is unsubstituted pyridyl. In embodiments, L³ is substituted (e.g., with a substituent group, a size-limited substituent group or a lower substituent group) or unsubstituted —CH₂NCH₂-(pyridyl). In embodiments, L³ is substituted (e.g., with a substituent group, a size-limited substituent group or a lower substituent group) —CH₂NCH₂-(pyridyl). In embodiments, L³ is unsubstituted —CH₂NCH₂-(pyridyl). In embodiments, L³ is substituted (e.g., with a substituent group, a size-limited substituent group or a lower substituent group) or unsubstituted —OCH₂-(pyridyl). In embodiments, L³ is substituted (e.g., with a substituent group, a size-limited substituent group or a lower substituent group) —OCH₂-(pyridyl). In embodiments, L³ is unsubstituted —OCH₂-(pyridyl).

In embodiments, L³ is substituted (e.g., with a substituent group, a size-limited substituent group or a lower substituent group) or unsubstituted pyranylene. In embodiments, L³ is substituted (e.g., with a substituent group, a size-limited substituent group or a lower substituent group) pyranylene. In embodiments, L³ is unsubstituted pyranylene. In embodiments, L³ is substituted (e.g., with a substituent group, a size-limited substituent group or a lower substituent group) or unsubstituted pyranyl. In embodiments, L³ is substituted (e.g., with a substituent group, a size-limited substituent group or a lower substituent group) pyranyl. In embodiments, L³ is unsubstituted pyranyl. In embodiments, L³ is substituted (e.g., with a substituent group, a size-limited substituent group or a lower substituent group) or unsubstituted —CH₂NCH₂-(pyranyl). In embodiments, L³ is substituted (e.g., with a substituent group, a size-limited substituent group or a lower substituent group) —CH₂NCH₂-(pyranyl). In embodiments, L³ is unsubstituted —CH₂NCH₂-(pyranyl). In embodiments, L³ is substituted (e.g., with a substituent group, a size-limited substituent group or a lower substituent group) or unsubstituted —OCH₂-(pyranyl). In embodiments, L³ is substituted (e.g., with a substituent group, a size-limited substituent group or a lower substituent group) —OCH₂-(pyranyl). In embodiments, L³ is unsubstituted —OCH₂-(pyranyl).

In embodiments, L³ is substituted (e.g., with a substituent group, a size-limited substituent group or a lower substituent group) or unsubstituted imidazolylene. In embodiments, L³ is substituted (e.g., with a substituent group, a size-limited substituent group or a lower substituent group) imidazolylene. In embodiments, L³ is unsubstituted imidazolylene. In embodiments, L³ is substituted (e.g., with a substituent group, a size-limited substituent group or a lower substituent group) or unsubstituted imidazolyl. In embodiments, L³ is substituted (e.g., with a substituent group, a size-limited substituent group or a lower substituent group) imidazolyl. In embodiments, L³ is unsubstituted imidazolyl. In embodiments, L³ is substituted (e.g., with a substituent group, a size-limited substituent group or a lower substituent group) or unsubstituted —CH₂NCH₂-(imidazolyl). In embodiments, L³ is substituted (e.g., with a substituent group, a size-limited substituent group or a lower substituent group) —CH₂NCH₂-(imidazolyl). In embodiments, L³ is unsubstituted —CH₂NCH₂-(imidazolyl). In embodiments, L³ is substituted (e.g., with a substituent group, a size-limited substituent group or a lower substituent group) or unsubstituted —OCH₂-(imidazolyl). In embodiments, L³ is substituted (e.g., with a substituent group, a size-limited substituent group or a lower substituent group) —OCH₂-(imidazolyl). In embodiments, L³ is unsubstituted —OCH₂-(imidazolyl).

In embodiments, L³ is substituted (e.g., with a substituent group, a size-limited substituent group or a lower substituent group) or unsubstituted thiazolylene. In embodiments, L³ is substituted (e.g., with a substituent group, a size-limited substituent group or a lower substituent group) thiazolylene. In embodiments, L³ is unsubstituted thiazolylene. In embodiments, L³ is substituted (e.g., with a substituent group, a size-limited substituent group or a lower substituent group) or unsubstituted thiazolyl. In embodiments, L³ is substituted (e.g., with a substituent group, a size-limited substituent group or a lower substituent group) thiazolyl. In embodiments, L³ is unsubstituted thiazolyl. In embodiments, L³ is substituted (e.g., with a substituent group, a size-limited substituent group or a lower substituent group) or unsubstituted —CH₂NCH₂-(thiazolyl). In embodiments, L³ is substituted (e.g., with a substituent group, a size-limited substituent group or a lower substituent group) —CH₂NCH₂-(thiazolyl). In embodiments, L³ is unsubstituted —CH₂NCH₂-(thiazolyl). In embodiments, L³ is substituted (e.g., with a substituent group, a size-limited substituent group or a lower substituent group) or unsubstituted —OCH₂-(thiazolyl). In embodiments, L³ is substituted (e.g., with a substituent group, a size-limited substituent group or a lower substituent group) —OCH₂-(thiazolyl). In embodiments, L³ is unsubstituted —OCH₂-(thiazolyl).

In embodiments, L³ is substituted (e.g., with a substituent group, a size-limited substituent group or a lower substituent group) or unsubstituted thienylene. In embodiments, L³ is substituted (e.g., with a substituent group, a size-limited substituent group or a lower substituent group) thienylene. In embodiments, L³ is unsubstituted thienylene. In embodiments, L³ is substituted (e.g., with a substituent group, a size-limited substituent group or a lower substituent group) or unsubstituted thienyl. In embodiments, L³ is substituted (e.g., with a substituent group, a size-limited substituent group or a lower substituent group) thienyl. In embodiments, L³ is unsubstituted thienyl. In embodiments, L³ is substituted (e.g., with a substituent group, a size-limited substituent group or a lower substituent group) or unsubstituted —CH₂NCH₂-(thienyl). In embodiments, L³ is substituted (e.g., with a substituent group, a size-limited substituent group or a lower substituent group) —CH₂NCH₂-(thienyl). In embodiments, L³ is unsubstituted —CH₂NCH₂-(thienyl). In embodiments, L³ is substituted (e.g., with a substituent group, a size-limited substituent group or a lower substituent group) or unsubstituted —OCH₂-(thienyl). In embodiments, L³ is substituted (e.g., with a substituent group, a size-limited substituent group or a lower substituent group) —OCH₂-(thienyl). In embodiments, L³ is unsubstituted —OCH₂-(thienyl).

In embodiments, L³ is substituted (e.g., with a substituent group, a size-limited substituent group or a lower substituent group) or unsubstituted oxazolylene. In embodiments, L³ is substituted (e.g., with a substituent group, a size-limited substituent group or a lower substituent group) oxazolylene. In embodiments, L³ is unsubstituted oxazolylene. In embodiments, L³ is substituted (e.g., with a substituent group, a size-limited substituent group or a lower substituent group) or unsubstituted oxazolyl. In embodiments, L³ is substituted (e.g., with a substituent group, a size-limited substituent group or a lower substituent group) oxazolyl. In embodiments, L³ is unsubstituted oxazolyl. In embodiments, L³ is unsubstituted oxazolylene. In embodiments, L³ is substituted (e.g., with a substituent group, a size-limited substituent group or a lower substituent group) or unsubstituted —CH₂NCH₂-(oxazolyl). In embodiments, L³ is substituted (e.g., with a substituent group, a size-limited substituent group or a lower substituent group) —CH₂NCH₂-(oxazolyl). In embodiments, L³ is unsubstituted —CH₂NCH₂-(oxazolyl). In embodiments, L³ is substituted (e.g., with a substituent group, a size-limited substituent group or a lower substituent group) or unsubstituted —OCH₂-(oxazolyl). In embodiments, L³ is substituted (e.g., with a substituent group, a size-limited substituent group or a lower substituent group) —OCH₂-(oxazolyl). In embodiments, L³ is unsubstituted —OCH₂-(oxazolyl).

In embodiments, R¹ is substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, R¹ is substituted with one or more substituent groups. In embodiments, R¹ is substituted with one or more size-limited substituent groups. In embodiments, R¹ is substituted with one or more lower substituent groups.

In embodiments, R¹ is substituted (e.g., with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R¹ is unsubstituted heterocycloalkyl (e.g., 3 to 8 membered heterocycloalkyl, 3 to 6 membered heterocycloalkyl, or 5 to 6 membered heterocycloalkyl). In embodiments, R¹ is substituted (e.g., with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R¹ is unsubstituted heteroaryl (e.g., 5 to 10 membered heteroaryl, 5 to 9 membered heteroaryl, or 5 to 6 membered heteroaryl).

In embodiments, R¹ is substituted (e.g., with a substituent group, a size-limited substituent group or a lower substituent group) or unsubstituted 3 to 8 membered heterocycloalkyl. In embodiments, R¹ is substituted (e.g., with a substituent group, a size-limited substituent group or a lower substituent group) 3 to 8 membered heterocycloalkyl. In embodiments, R¹ is unsubstituted 3 to 8 membered heterocycloalkyl.

In embodiments, R¹ is substituted (e.g., with a substituent group, a size-limited substituent group or a lower substituent group) or unsubstituted 3 to 6 membered heterocycloalkyl. In embodiments, R¹ is substituted (e.g., with a substituent group, a size-limited substituent group or a lower substituent group) 3 to 6 membered heterocycloalkyl. In embodiments, R¹ is unsubstituted 3 to 6 membered heterocycloalkyl.

In embodiments, R¹ is substituted (e.g., with a substituent group, a size-limited substituent group or a lower substituent group) or unsubstituted heterocyclobutyl, heterocyclopentyl or heterocyclohexyl. In embodiments, R^{t} is substituted (e.g., with a substituent group, a size-limited substituent group or a lower substituent group) heterocyclobutyl, heterocyclopentyl or heterocyclohexyl. In embodiments, R¹ is unsubstituted heterocyclobutyl, heterocyclopentyl or heterocyclohexyl.

In embodiments, R¹ is substituted (e.g., with a substituent group, a size-limited substituent group or a lower substituent group) or unsubstituted heterocyclobutyl. In embodiments, R¹ is substituted (e.g., with a substituent group, a size-limited substituent group or a lower substituent group) heterocyclobutyl. In embodiments, R¹ is unsubstituted heterocyclobutyl.

In embodiments, R¹ is substituted (e.g., with a substituent group, a size-limited substituent group or a lower substituent group) or unsubstituted heterocyclopentyl. In embodiments, R¹ is substituted (e.g., with a substituent group, a size-limited substituent group or a lower substituent group) heterocyclopentyl. In embodiments, R¹ is unsubstituted heterocyclopentyl.

In embodiments, R¹ is substituted (e.g., with a substituent group, a size-limited substituent group or a lower substituent group) or unsubstituted heterocyclohexyl. In embodiments, R¹ is substituted (e.g., with a substituent group, a size-limited substituent group or a lower substituent group) heterocyclohexyl. In embodiments, R¹ is unsubstituted heterocyclohexyl.

In embodiments, R¹ is substituted (e.g., with a substituent group, a size-limited substituent group or a lower substituent group) or unsubstituted 5 to 10 membered heteroaryl. In embodiments, R¹ is substituted (e.g., with a substituent group, a size-limited substituent group or a lower substituent group) 5 to 10 membered heteroaryl. In embodiments, R¹ is unsubstituted 5 to 10 membered heteroaryl.

In embodiments, R¹ is substituted (e.g., with a substituent group, a size-limited substituent group or a lower substituent group) or unsubstituted 5 to 9 membered heteroaryl. In embodiments, R¹ is substituted (e.g., with a substituent group, a size-limited substituent group or a lower substituent group) 5 to 9 membered heteroaryl. In embodiments, R¹ is unsubstituted 5 to 9 membered heteroaryl.

In embodiments, R¹ is substituted (e.g., with a substituent group, a size-limited substituent group or a lower substituent group) or unsubstituted 5 to 6 membered heteroaryl. In embodiments, R¹ is substituted (e.g., with a substituent group, a size-limited substituent group or a lower substituent group) 5 to 6 membered heteroaryl. In embodiments, R¹ is unsubstituted 5 to 6 membered heteroaryl.

In embodiments, R¹ is substituted (e.g., with a substituent group, a size-limited substituent group or a lower substituent group) or unsubstituted furanyl, pyrrolyl, pyridyl, pyranyl, imidazolyl, or thiazolyl. In embodiments, R¹ is substituted (e.g., with a substituent group, a size-limited substituent group or a lower substituent group) furanyl, pyrrolyl, pyridyl, pyranyl, imidazolyl, or thiazolyl. In embodiments, R¹ is unsubstituted furanyl, pyrrolyl, pyridyl, pyranyl, imidazolyl, or thiazolyl.

In embodiments, R¹ is substituted (e.g., with a substituent group, a size-limited substituent group or a lower substituent group) or unsubstituted furanyl. In embodiments, R′ is substituted (e.g., with a substituent group, a size-limited substituent group or a lower substituent group) furanyl. In embodiments, R¹ is unsubstituted furanyl.

In embodiments, R¹ is substituted (e.g., with a substituent group, a size-limited substituent group or a lower substituent group) or unsubstituted pyrrolyl. In embodiments, R′ is substituted (e.g., with a substituent group, a size-limited substituent group or a lower substituent group) pyrrolyl. In embodiments, R¹ is unsubstituted pyrrolyl.

In embodiments, R¹ is substituted (e.g., with a substituent group, a size-limited substituent group or a lower substituent group) or unsubstituted pyridyl. In embodiments, R¹ is substituted (e.g., with a substituent group, a size-limited substituent group or a lower substituent group) pyridyl. In embodiments, R¹ is unsubstituted pyridyl.

In embodiments, R¹ is substituted (e.g., with a substituent group, a size-limited substituent group or a lower substituent group) or unsubstituted pyranyl. In embodiments, R¹ is substituted (e.g., with a substituent group, a size-limited substituent group or a lower substituent group) pyranyl. In embodiments, R¹ is unsubstituted pyranyl.

In embodiments, R¹ is substituted (e.g., with a substituent group, a size-limited substituent group or a lower substituent group) or unsubstituted imidazolyl. In embodiments, R′ is substituted (e.g., with a substituent group, a size-limited substituent group or a lower substituent group) imidazolyl. In embodiments, R¹ is unsubstituted imidazolyl.

In embodiments, R¹ is substituted (e.g., with a substituent group, a size-limited substituent group or a lower substituent group) or unsubstituted thiazolyl. In embodiments, R¹ is substituted (e.g., with a substituent group, a size-limited substituent group or a lower substituent group) thiazolyl. In embodiments, R¹ is unsubstituted thiazolyl.

In embodiments, provided herein is an ADC of formula (IA) or formula (IIA):

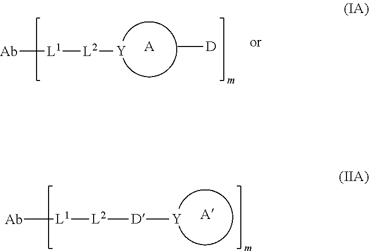

or a pharmaceutically acceptable salt thereof, wherein:
ring A is a substituted or unsubstituted heterocycloalkylene or a substituted or unsubstituted heteroarylene, connected to L² through a heteroatom Y;
ring A′ is a substituted or unsubstituted heterocycloalkyl or a substituted or unsubstituted heteroaryl, connected to D′ through a heteroatom Y;
each Y is independently N, P, or S; and
L¹, L², Ab, m, D, and D′ are each as defined herein including embodiments.

In embodiments, ring A is a substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene) or substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene). In embodiments, ring A is substituted with one or more substituent groups. In embodiments, ring A is substituted with one or more size-limited substituent groups. In embodiments, ring A is substituted with one or more lower substituent groups. Ring A is connected to L² through a heteroatom Y.

In embodiments, ring A′ is substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, ring A′ is substituted with one or more substituent groups. In embodiments, ring A′ is substituted with one or more size-limited substituent groups. In embodiments, ring A′ is substituted with one or more lower substituent groups. Ring A′ is connected to D′ through a heteroatom Y. In embodiments, each Y is N.

In embodiments, ring A is a substituted with one or more (e.g., with a substituent group, a size-limited substituent group or a lower substituent group) 3 to 8 membered heterocycloalkylene, where ring A is connected to L² through a heteroatom Y. In embodiments, ring A′ is a substituted with one or more (e.g., with a substituent group, a size-limited substituent group or a lower substituent group) 3 to 8 membered heterocycloalkyl, where ring A′ is connected to D′ through a heteroatom Y. In embodiments, each Y is N.

In embodiments, ring A is a substituted with one or more (e.g., with a substituent group, a size-limited substituent group or a lower substituent group) 5 to 6 membered heterocycloalkylene, where ring A is connected to L² through a heteroatom Y. In embodiments, ring A′ is a substituted with one or more (e.g., with a substituent group, a size-limited substituent group or a lower substituent group) 5 to 6 membered heterocycloalkyl, where ring A′ is connected to D′ through a heteroatom Y. In embodiments, each Y is N.

In embodiments, provided herein is an ADC of formula (IB) or formula (IIB):

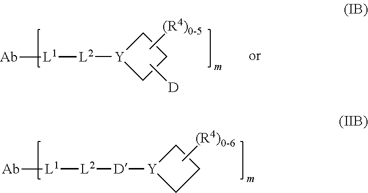

or a pharmaceutically acceptable salt thereof, wherein:
each R⁴ is independently H, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A}, —POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   any two R⁴ substituents on adjacent carbon atoms may optionally be joined to form a substituted or unsubstituted cycloalkyl, substituted or unsubstituted heterocycloalkyl, substituted or unsubstituted aryl or substituted or unsubstituted heteroaryl;
each R^{4A} and R^{4B} is independently H, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X is —Cl, —Br, —I or —F;
each n4 is independently an integer from 0 to 4;
each v4 is independently 1 or 2;
each m4 is independently an integer from 0 to 3; and
each r4 is independently 1 or 2; and
Y, m, D, D′, L¹, L² and Ab are each as defined herein including embodiments.

In embodiments, each R⁴ is independently H, halogen, or substituted or unsubstituted alkyl. In embodiments, each R⁴ is independently H, chloro, bromo, iodo, fluoro, or substituted or unsubstituted alkyl. In embodiments, each R⁴ is independently H, chloro, bromo, iodo, fluoro, methyl, ethyl, propyl, isopropyl, butyl, isobutyl, tert-butyl, pentyl, isopentyl, or hexyl. In embodiments, each R⁴ is independently H. In embodiments, each R⁴ is independently fluoro. In embodiments, each R⁴ is independently methyl. In embodiments, each R⁴ is independently ethyl.

In embodiments, provided herein is an ADC of formula (IC) or formula (IIC):

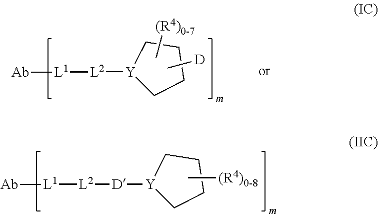

or a pharmaceutically acceptable salt thereof; wherein D, D′, m, Y, L¹, L² R⁴, and Ab are each as defined herein including embodiments.

In embodiments, provided herein is an ADC of formula (ID) or formula (IID):

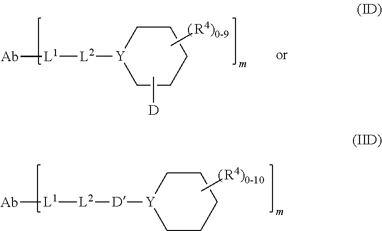

or a pharmaceutically acceptable salt thereof; wherein D, D′, m, Y, L¹, L² R⁴, and Ab are each as defined herein including embodiments.

In embodiments, provided herein is an ADC of formula (ID1) or formula (IID1):

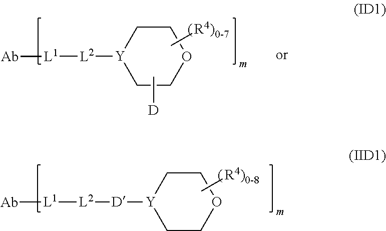

or a pharmaceutically acceptable salt thereof; wherein D, D′, m, Y, L¹, L² R⁴, and Ab are each as defined herein including embodiments.

In embodiments, provided herein is an ADC of formula (IE) or formula (IIE):

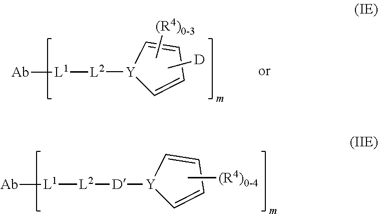

or a pharmaceutically acceptable salt thereof; wherein D, D′, m, Y, L¹, L² R⁴, and Ab are each as defined herein including embodiments.

In embodiments, provided herein is an ADC of formula (IF) or formula (IIF):

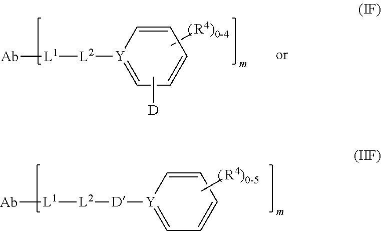

or a pharmaceutically acceptable salt thereof; wherein D, D′, m, Y, L¹, L² R⁴, and Ab are each as defined herein including embodiments.

In embodiments, provided herein is an ADC of formula (IG) or formula (IH):

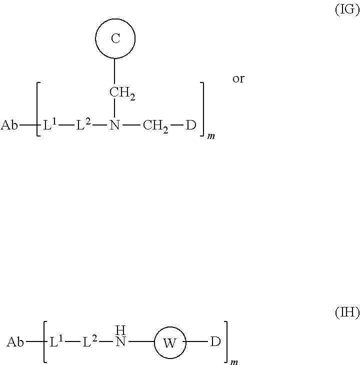

or a pharmaceutically acceptable salt thereof, wherein:
ring W is a substituted or unsubstituted cycloalkylene or a substituted or unsubstituted arylene;
ring C is a substituted or unsubstituted cycloalkyl, substituted or unsubstituted heterocycloalkyl, substituted or unsubstituted aryl, or substituted or unsubstituted heteroaryl; and wherein D, m, L¹, L², and Ab are each as defined herein including embodiments.

In embodiments, ring W is a substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene) or substituted (e.g. with a substituent group, a size-limited substituent group or a lower substituent group) or unsubstituted arylene (e.g., C₅-C₁₀ arylene, C₅-C₅ arylene, or C₅-C₆ arylene). In embodiments, ring W is substituted with one or more substituent groups. In embodiments, ring W is substituted with one or more size-limited substituent groups. In embodiments, ring W is substituted with one or more lower substituent groups.

In embodiments, ring W is a substituted (e.g., with a substituent group, a size-limited substituent group or a lower substituent group) or unsubstituted C₃-C₈ cycloalkylene. In embodiments, ring W is a substituted (e.g., with a substituent group, a size-limited substituent group or a lower substituent group) C₃-C₈ cycloalkylene. In embodiments, ring W is an unsubstituted C₃-C₈ cycloalkylene.

In embodiments, ring W is a substituted with one or more (e.g., with a substituent group, a size-limited substituent group or a lower substituent group) C₃-C₈ cycloalkylene.

In embodiments, ring W is a substituted (e.g., with a substituent group, a size-limited substituent group or a lower substituent group) or unsubstituted cyclobutylene. In embodiments, ring W is a substituted (e.g., with a substituent group, a size-limited substituent group or a lower substituent group) or unsubstituted cyclopentylene. In embodiments, ring W is a substituted (e.g., with a substituent group, a size-limited substituent group or a lower substituent group) or unsubstituted cyclohexylene.

In embodiments, ring W is a substituted (e.g., with a substituent group, a size-limited substituent group or a lower substituent group) or unsubstituted C₅-C₆ arylene. In embodiments, ring W is a substituted (e.g., with a substituent group, a size-limited substituent group or a lower substituent group) C₅-C₆ arylene. In embodiments, ring W is an unsubstituted C₅-C₆ arylene. In embodiments, ring W is a substituted with one or more (e.g., with a substituent group, a size-limited substituent group or a lower substituent group) C₅-C₆ arylene.

In embodiments, ring C is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, ring C is substituted with one or more substituent groups. In embodiments, ring C is substituted with one or more size-limited substituent groups. In embodiments, ring C is substituted with one or more lower substituent groups.

In embodiments, ring C is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, ring C is substituted with one or more substituent groups. In embodiments, ring C is substituted with one or more size-limited substituent groups. In embodiments, ring C is substituted with one or more lower substituent groups.

In embodiments, ring C is a substituted with one or more (e.g., with a substituent group, a size-limited substituent group or a lower substituent group) 5 to 9 membered heteroaryl. In embodiments, ring C is an unsubstituted 5 to 9 membered heteroaryl.

In embodiments, ring C is a substituted with one or more (e.g., with a substituent group, a size-limited substituent group or a lower substituent group) 5 to 6 membered heteroaryl. In embodiments, ring C is an unsubstituted 5 to 6 membered heteroaryl.

In embodiments, ring C is a substituted with one or more (e.g., with a substituent group, a size-limited substituent group or a lower substituent group) 3 to 8 membered heterocycloalkyl. In embodiments, ring C is a substituted with one or more (e.g., with a substituent group, a size-limited substituent group or a lower substituent group) 5 to 6 membered heterocycloalkyl.

In embodiments, ring C is substituted (e.g., with a substituent group, a size-limited substituent group or a lower substituent group) or unsubstituted furanyl, pyrrolyl, pyridyl, pyranyl, imidazolyl, thienyl, oxazolyl, or thiazolyl. In embodiments, ring C is substituted (e.g., with a substituent group, a size-limited substituent group or a lower substituent group) furanyl, pyrrolyl, pyridyl, pyranyl, imidazolyl, thienyl, oxazolyl, or thiazolyl. In embodiments, ring C is unsubstituted furanyl, pyrrolyl, pyridyl, pyranyl, imidazolyl, thienyl, oxazolyl, or thiazolyl.

In embodiments, ring C is substituted (e.g., with a substituent group, a size-limited substituent group or a lower substituent group) or unsubstituted furanyl. In embodiments, ring C is substituted (e.g., with a substituent group, a size-limited substituent group or a lower substituent group) furanyl. In embodiments, ring C is unsubstituted furanyl.

In embodiments, ring C is substituted (e.g., with a substituent group, a size-limited substituent group or a lower substituent group) or unsubstituted pyrrolyl. In embodiments, ring C is substituted (e.g., with a substituent group, a size-limited substituent group or a lower substituent group) pyrrolyl. In embodiments, ring C is unsubstituted pyrrolyl.

In embodiments, ring C is substituted (e.g., with a substituent group, a size-limited substituent group or a lower substituent group) or unsubstituted pyridyl. In embodiments, ring C is substituted (e.g., with a substituent group, a size-limited substituent group or a lower substituent group) pyridyl. In embodiments, ring C is unsubstituted pyridyl.

In embodiments, ring C is substituted (e.g., with a substituent group, a size-limited substituent group or a lower substituent group) or unsubstituted pyranyl. In embodiments, ring C is substituted (e.g., with a substituent group, a size-limited substituent group or a lower substituent group) pyranyl. In embodiments, ring C is unsubstituted pyranyl.

In embodiments, ring C is substituted (e.g., with a substituent group, a size-limited substituent group or a lower substituent group) or unsubstituted imidazolyl. In embodiments, ring C is substituted (e.g., with a substituent group, a size-limited substituent group or a lower substituent group) imidazolyl. In embodiments, ring C is unsubstituted imidazolyl.

In embodiments, ring C is substituted (e.g., with a substituent group, a size-limited substituent group or a lower substituent group) or unsubstituted thiazolyl. In embodiments, ring C is substituted (e.g., with a substituent group, a size-limited substituent group or a lower substituent group) thiazolyl. In embodiments, ring C is unsubstituted thiazolyl.

In embodiments, ring C is substituted (e.g., with a substituent group, a size-limited substituent group or a lower substituent group) or unsubstituted thienyl. In embodiments, ring C is substituted (e.g., with a substituent group, a size-limited substituent group or a lower substituent group) thienyl. In embodiments, ring C is unsubstituted thienyl.

In embodiments, ring C is substituted (e.g., with a substituent group, a size-limited substituent group or a lower substituent group) or unsubstituted oxazolyl. In embodiments, ring C is substituted (e.g., with a substituent group, a size-limited substituent group or a lower substituent group) oxazolyl. In embodiments, ring C is unsubstituted oxazolyl.

In embodiments, ring C is a substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl) or substituted (e.g. with a substituent group, a size-limited substituent group or a lower substituent group) or unsubstituted aryl (e.g., C₅-C₁₀ aryl, C₅-C₈ aryl, or C₅-C₆ aryl). In embodiments, ring C is substituted with one or more substituent groups. In embodiments, ring C is substituted with one or more size-limited substituent groups. In embodiments, ring C is substituted with one or more lower substituent groups.

In embodiments, ring C is a substituted (e.g., with a substituent group, a size-limited substituent group or a lower substituent group) or unsubstituted C₃-C₈ cycloalkyl. In embodiments, ring C is a substituted (e.g., with a substituent group, a size-limited substituent group or a lower substituent group) C₃-C₈ cycloalkyl. In embodiments, ring C is an unsubstituted C₃-C₈ cycloalkyl. In embodiments, ring C is a substituted with one or more (e.g., with a substituent group, a size-limited substituent group or a lower substituent group) C₃-C₈ cycloalkyl.

In embodiments, ring C is a substituted (e.g., with a substituent group, a size-limited substituent group or a lower substituent group) or unsubstituted cyclobutyl. In embodiments, ring C is a substituted (e.g., with a substituent group, a size-limited substituent group or a lower substituent group) or unsubstituted cyclopentyl. In embodiments, ring C is a substituted (e.g., with a substituent group, a size-limited substituent group or a lower substituent group) or unsubstituted cyclohexyl.

In embodiments, ring C is a substituted (e.g., with a substituent group, a size-limited substituent group or a lower substituent group) or unsubstituted C₅-C₆ aryl. In embodiments, ring C is a substituted (e.g., with a substituent group, a size-limited substituent group or a lower substituent group) C₅-C₆ aryl. In embodiments, ring C is an unsubstituted C₅-C₆ aryl. In embodiments, ring C is a substituted with one or more (e.g., with a substituent group, a size-limited substituent group or a lower substituent group) C₅-C₆ aryl.

In embodiments, provided herein is an ADC of formula (IJ) or formula (IK):

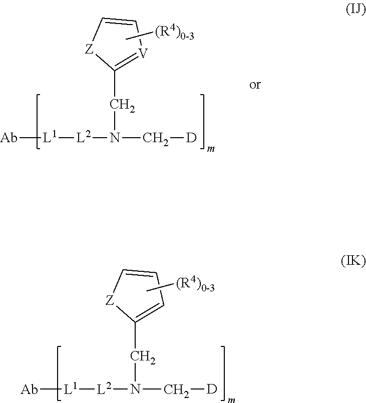

or a pharmaceutically acceptable salt thereof, wherein:
Z is S, N, or O; V is C or N; and wherein D, m, L¹, L², R⁴, and Ab are each as defined herein including embodiments.

In embodiments, Z is N. In embodiments, Z is O. In embodiments, Z is S.

In embodiments, V is C. In embodiments, V is N.

In embodiments, provided herein is an ADC of formula (IL) or formula (IM):

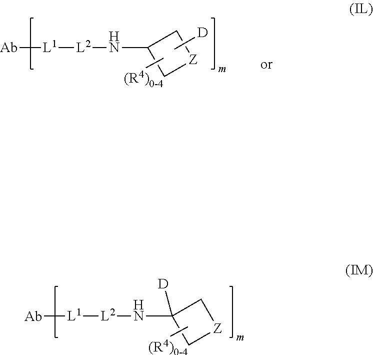

or a pharmaceutically acceptable salt thereof, wherein D, Z, m, L¹, L², R⁴, and Ab are each as defined herein including embodiments.

In embodiments, provided herein is an ADC of formula (IN) or formula (IO):

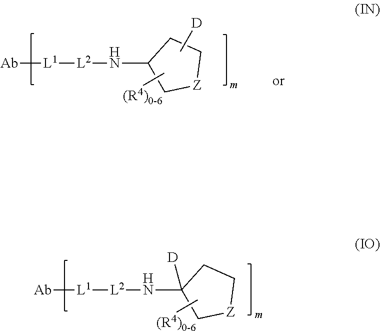

or a pharmaceutically acceptable salt thereof, wherein D, Z, m, L¹, L², R⁴, and Ab are each as defined herein including embodiments.

In embodiments, provided herein is an ADC of formula (TP) or formula (IQ):

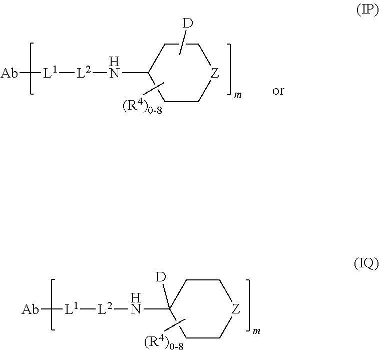

or a pharmaceutically acceptable salt thereof, wherein D, Z, m, L¹, L², R⁴, and Ab are each as defined herein including embodiments.

In embodiments, provided herein is an ADC having the structure:

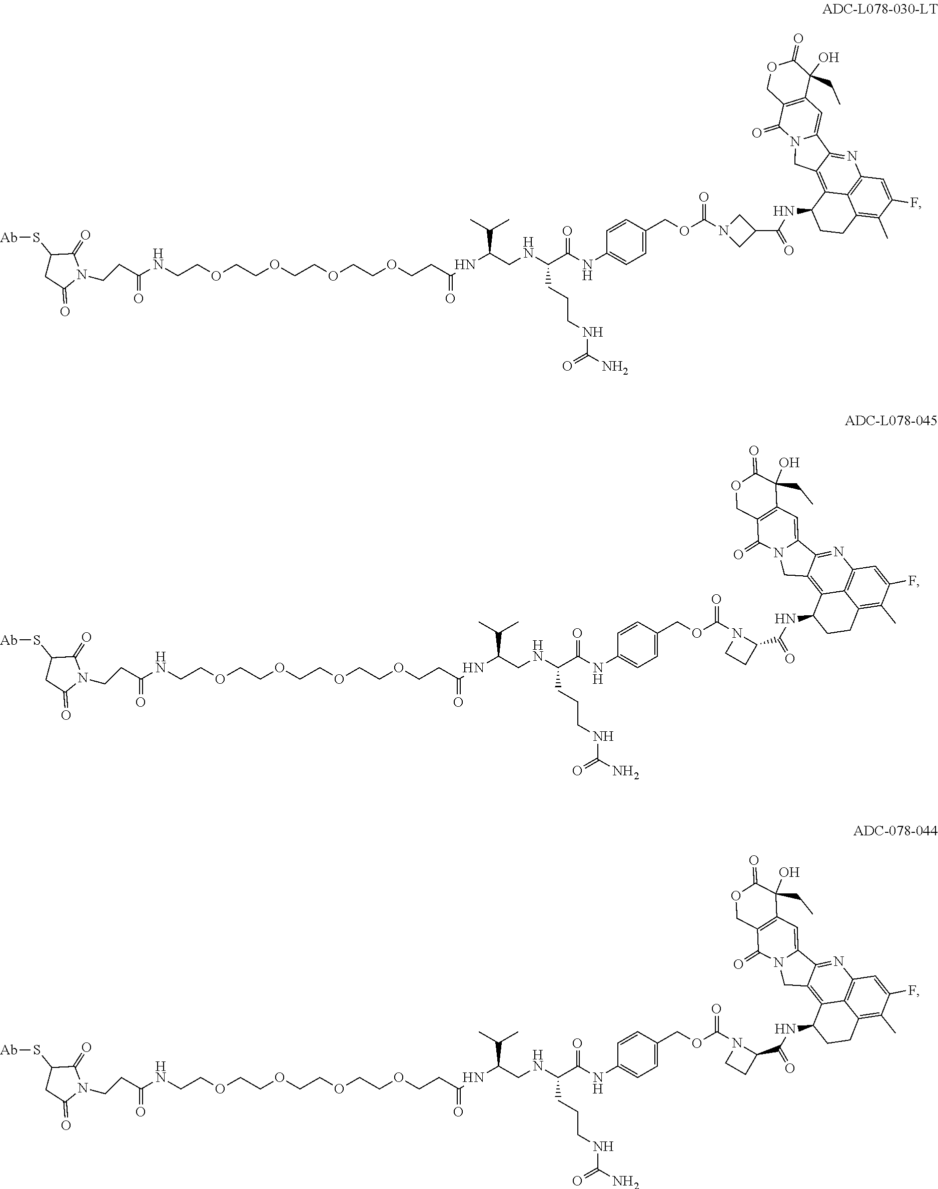

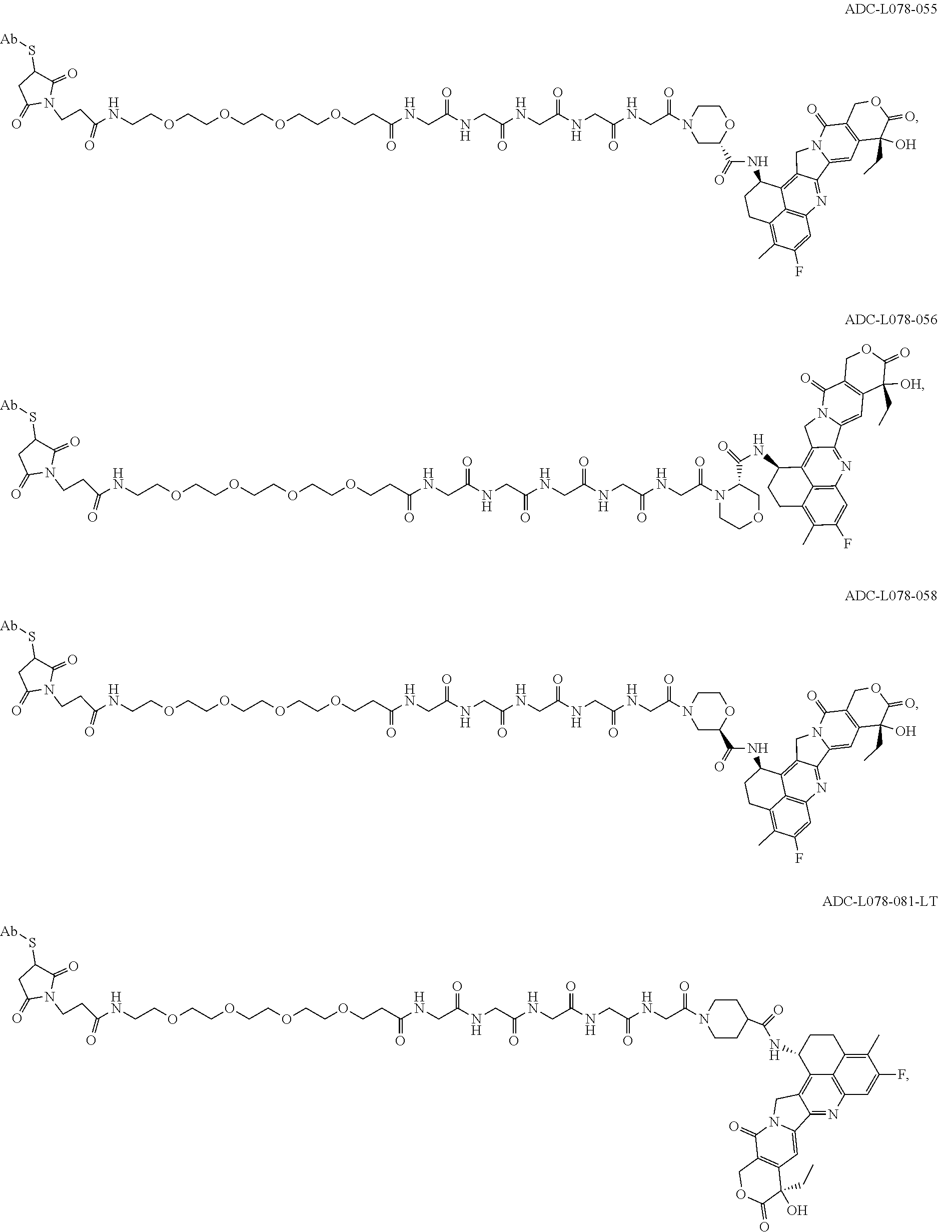

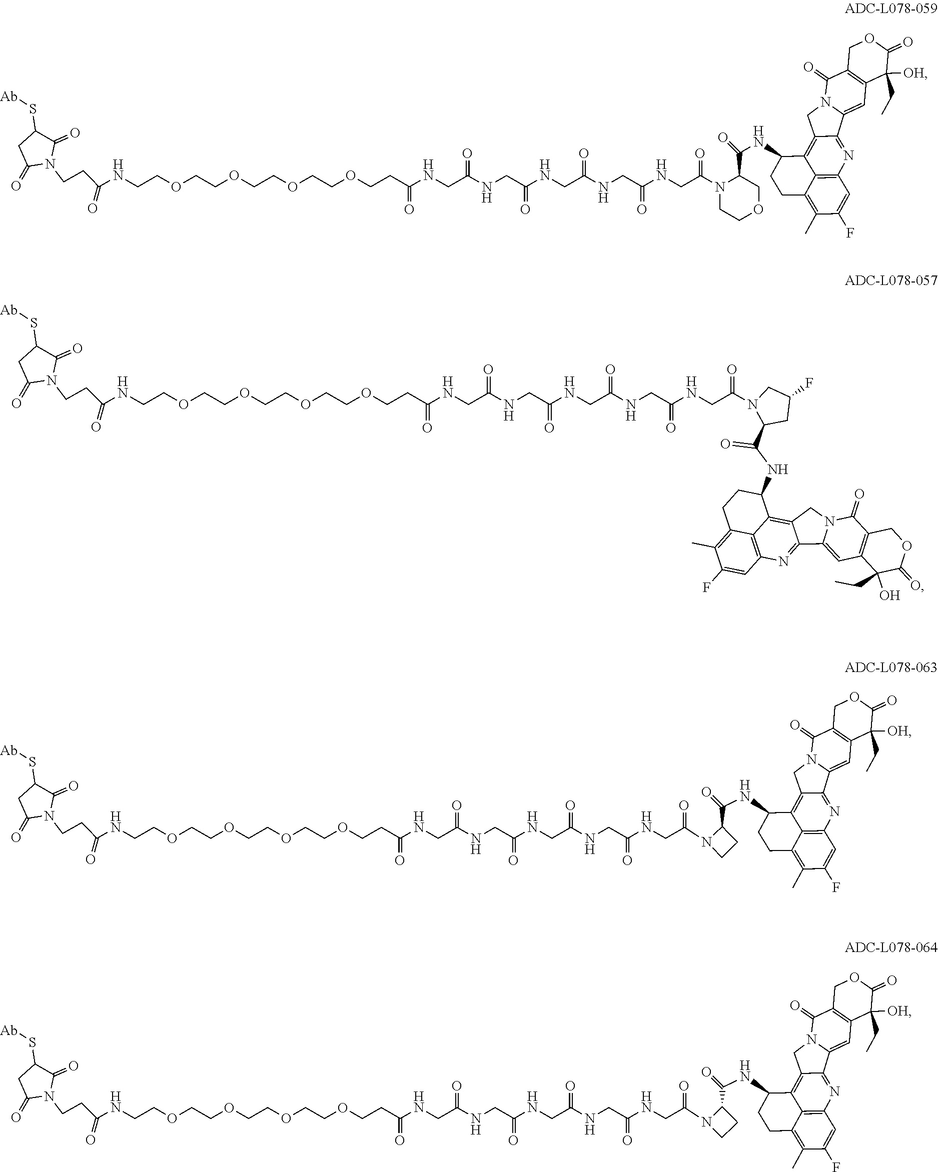

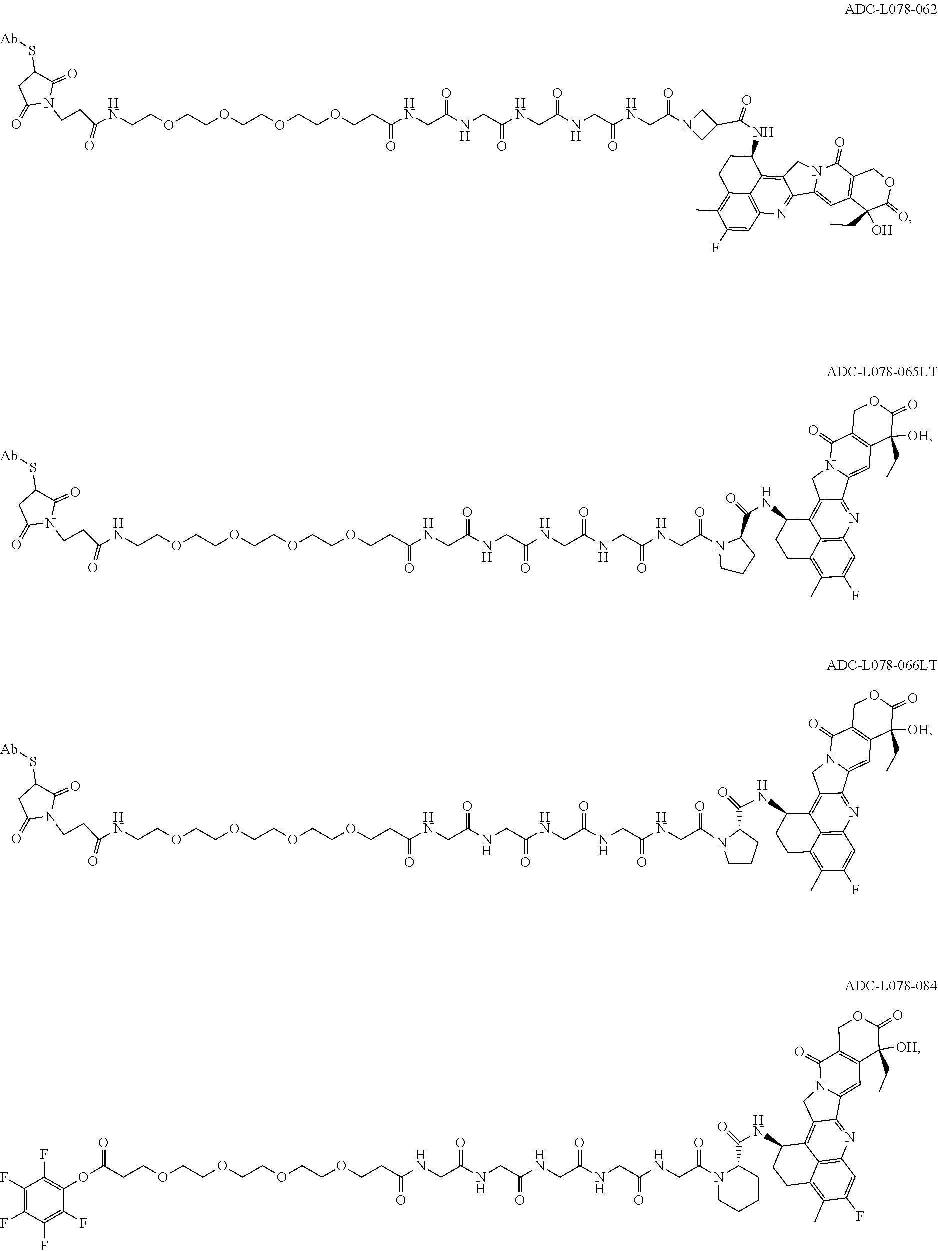

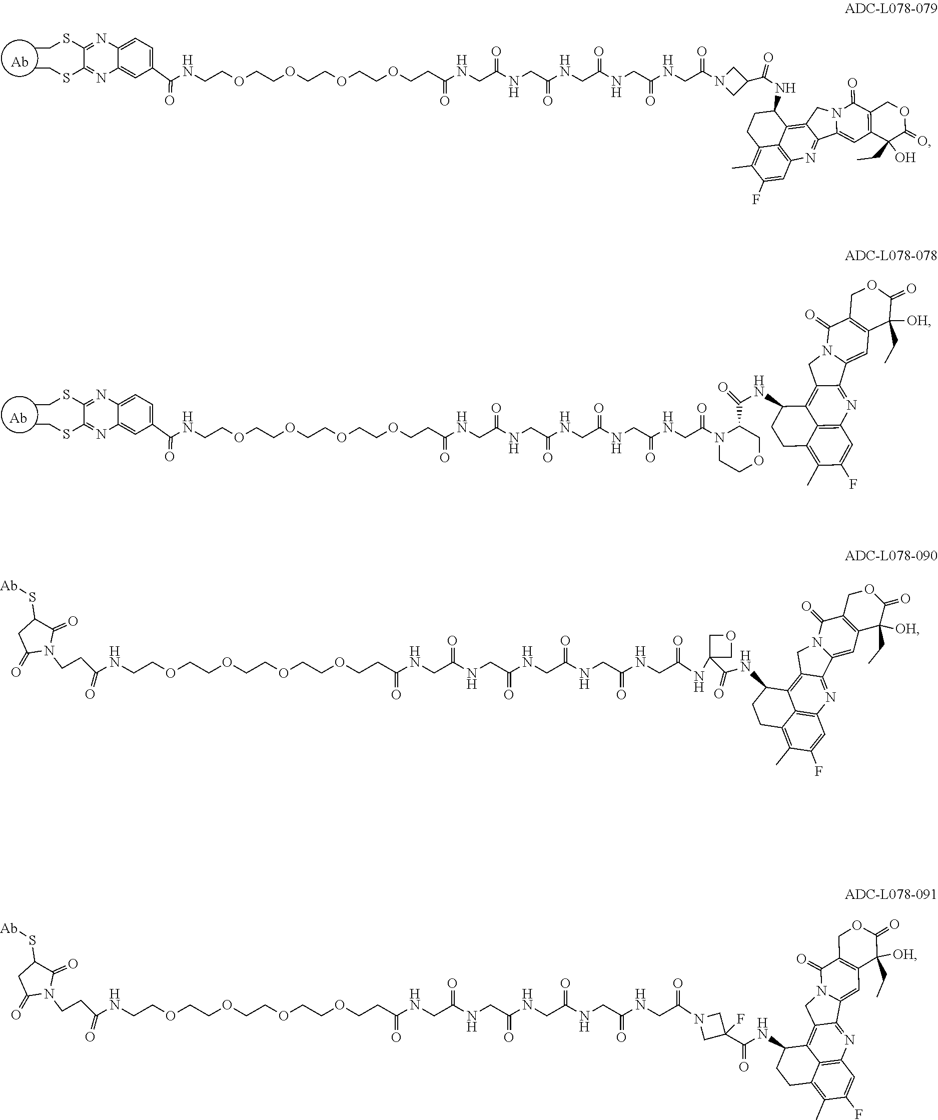

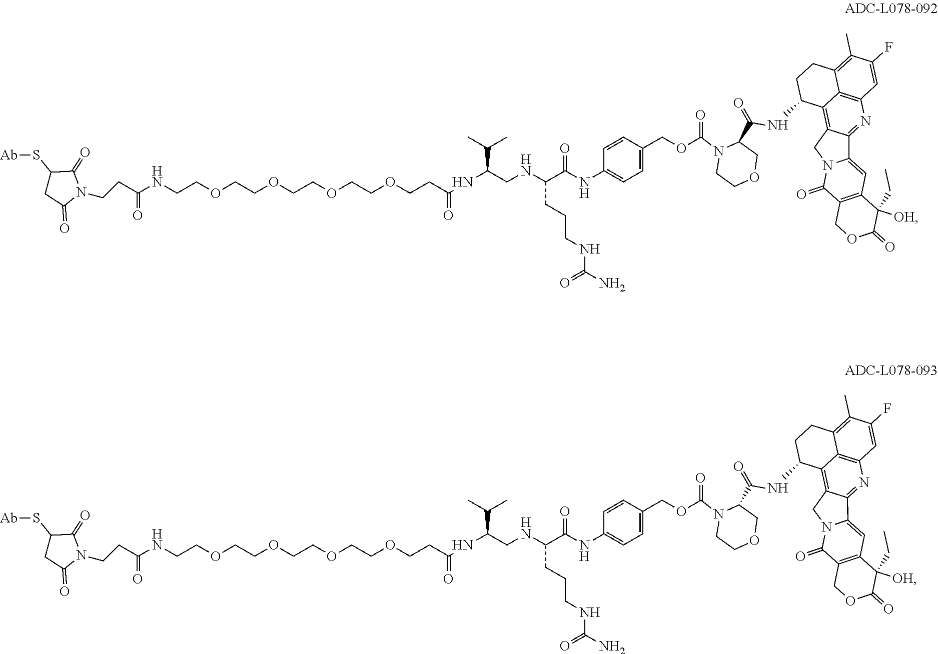

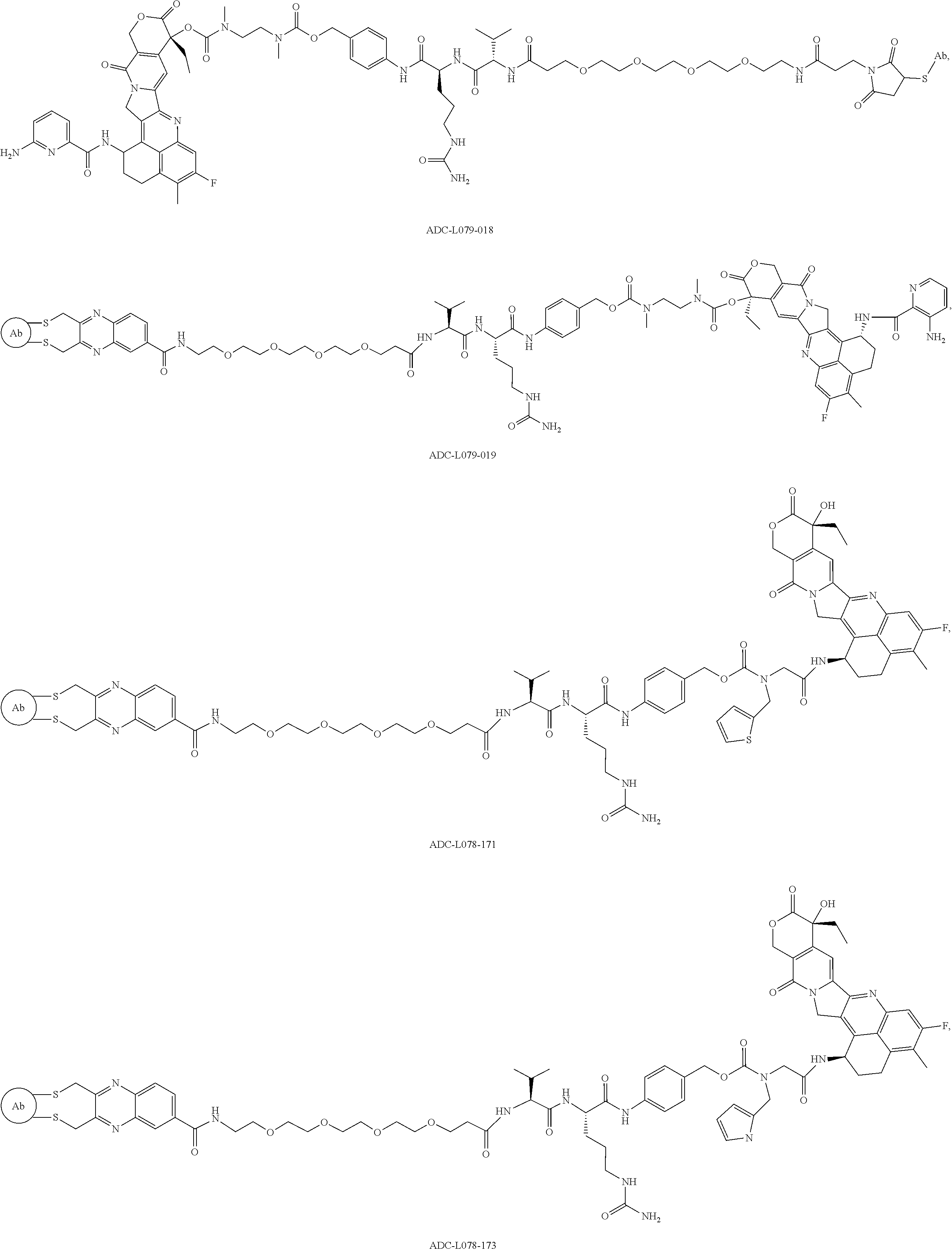

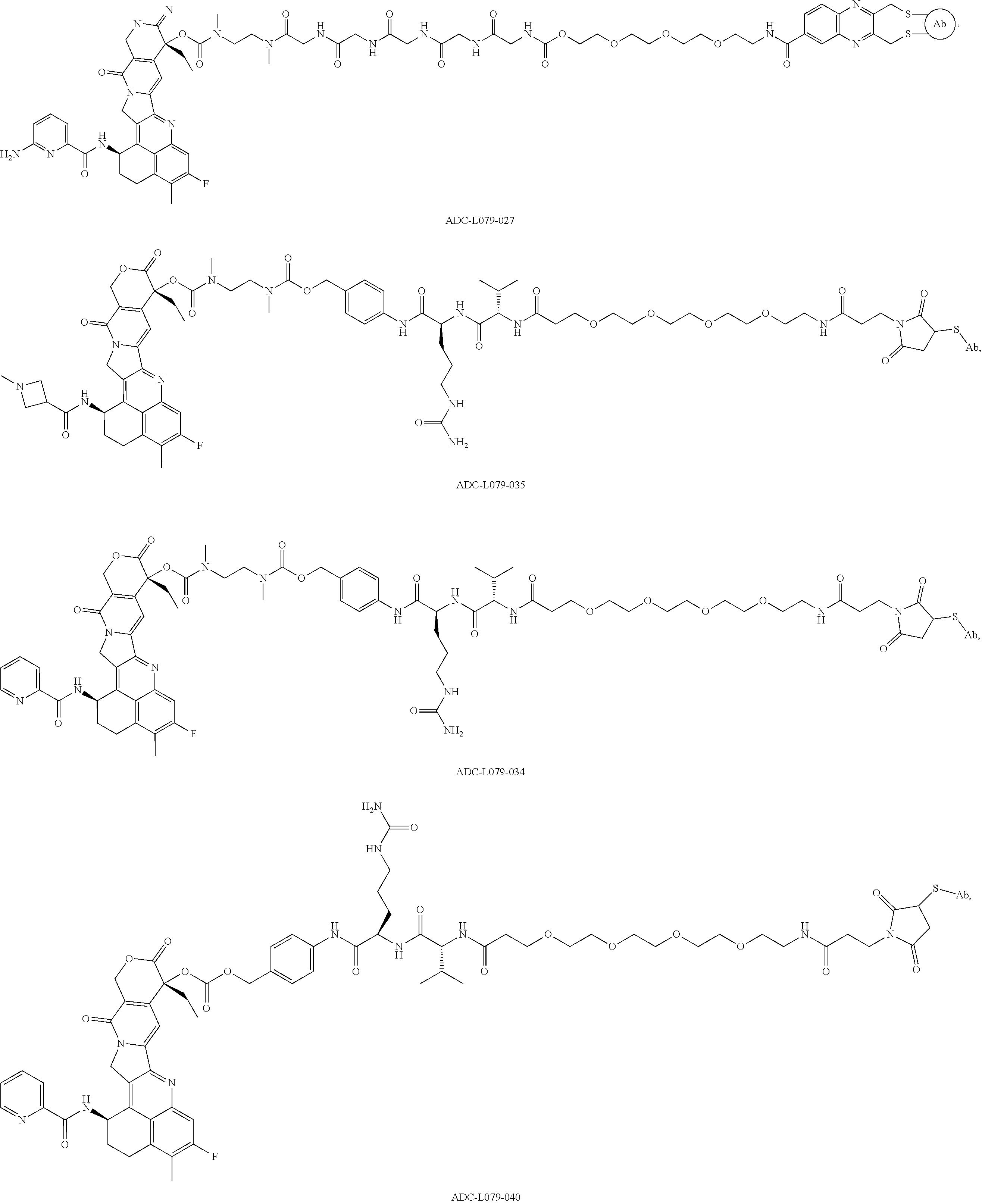

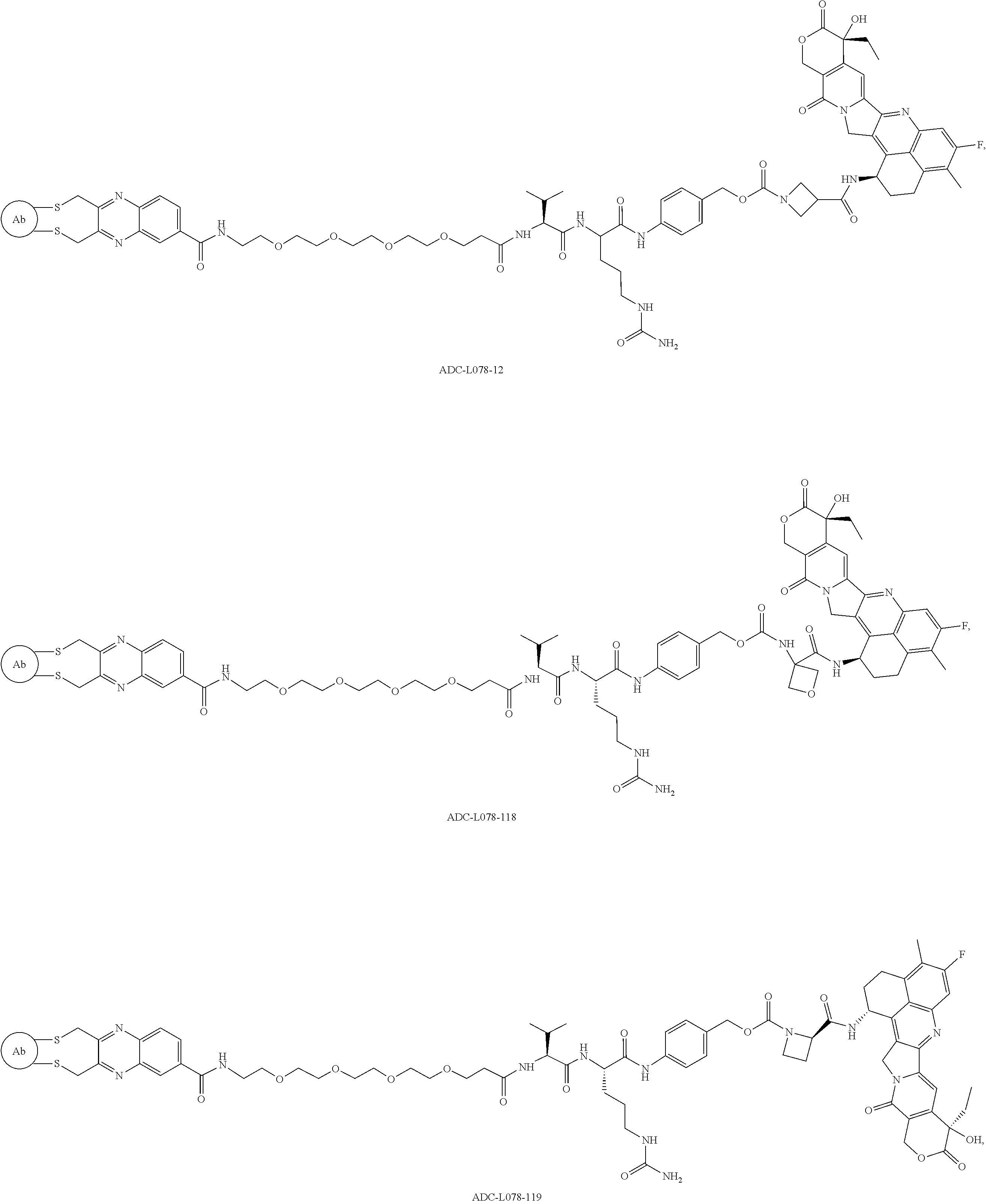

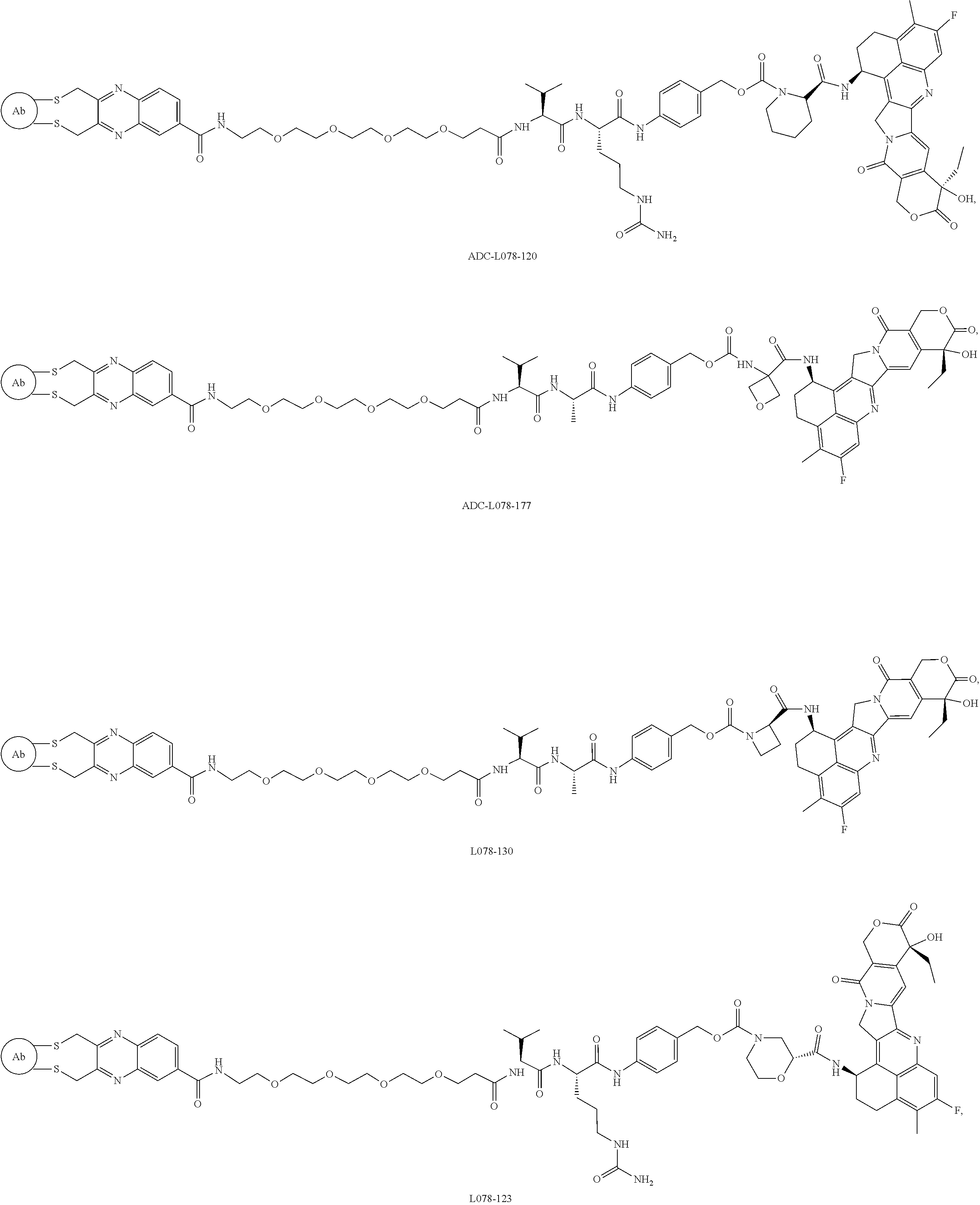

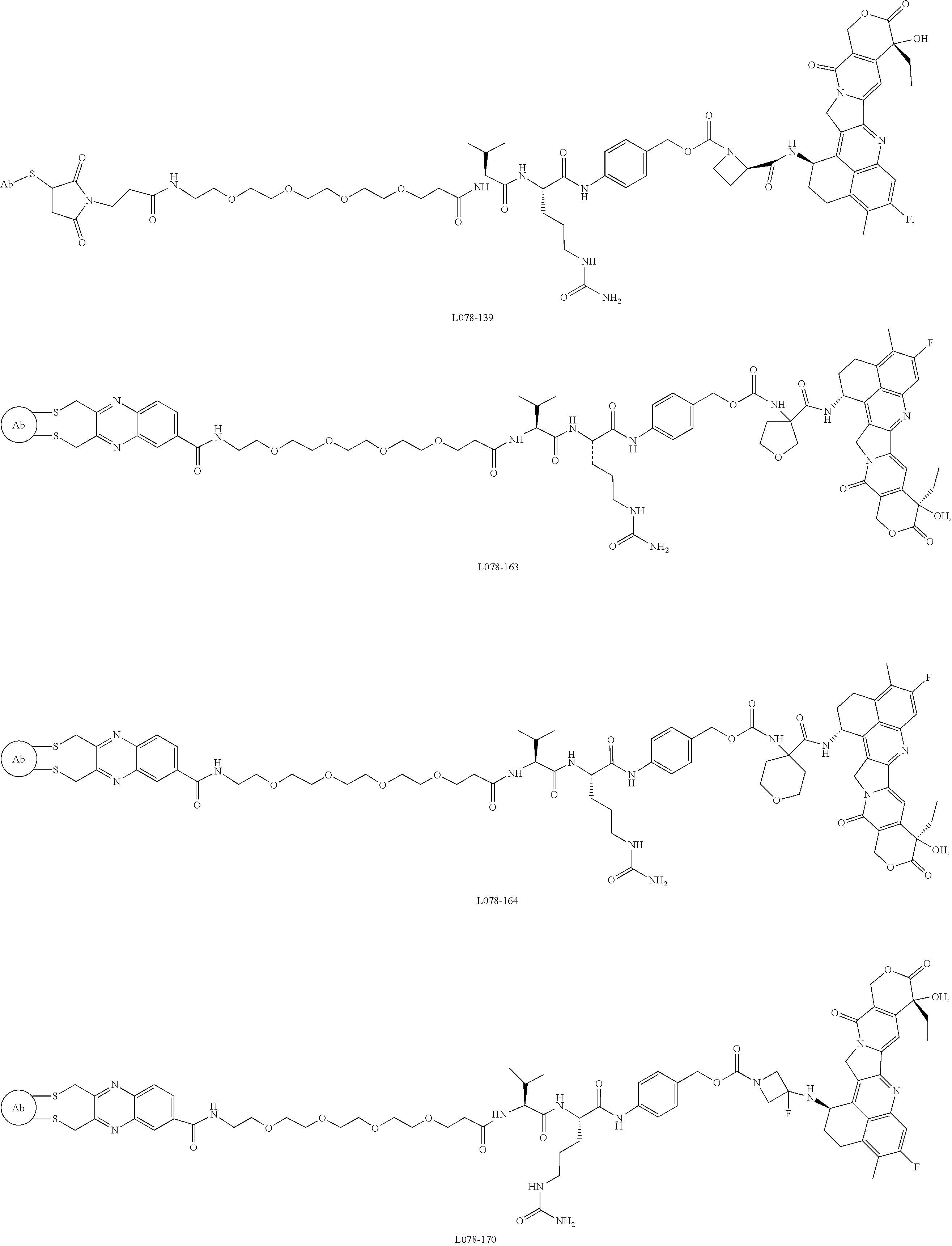

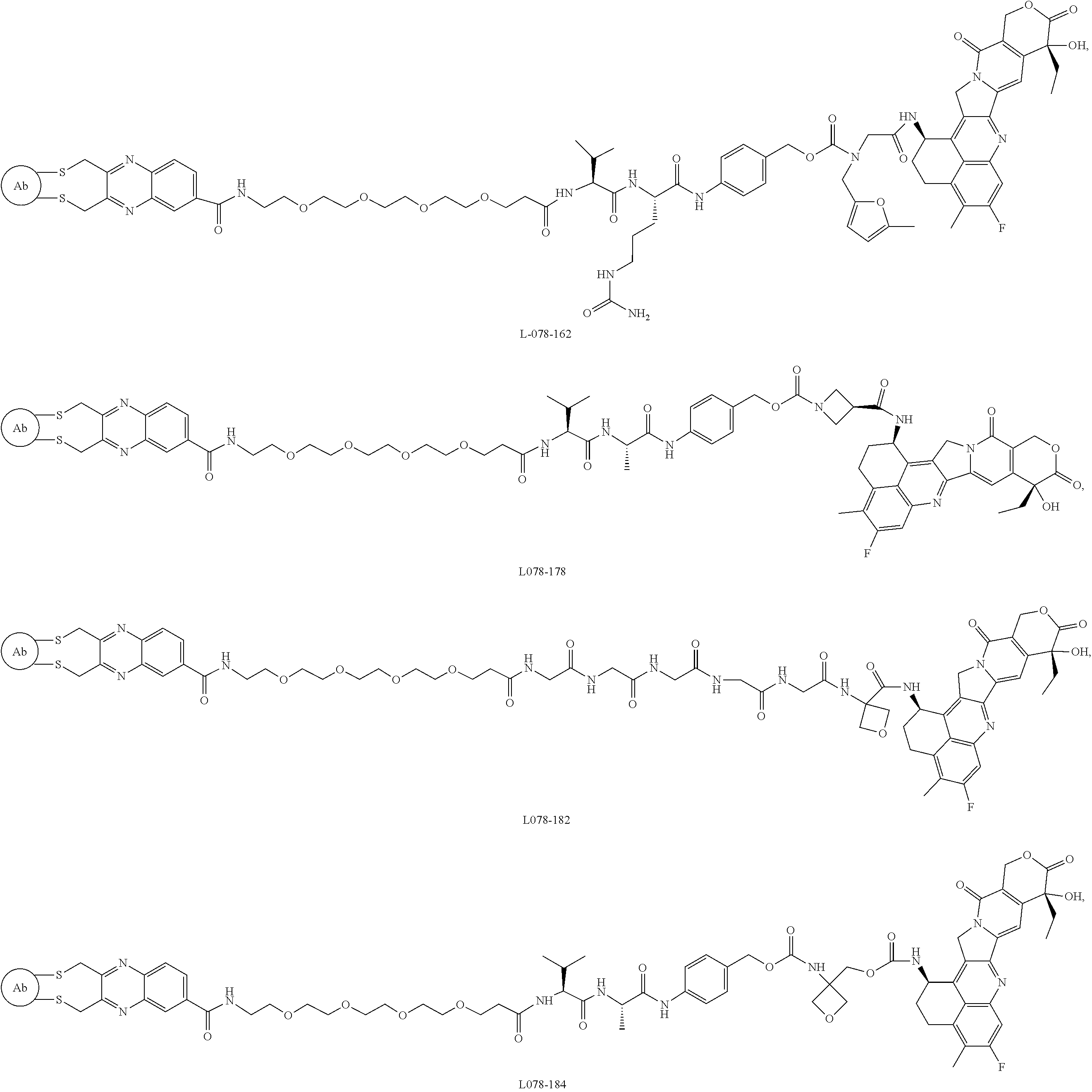

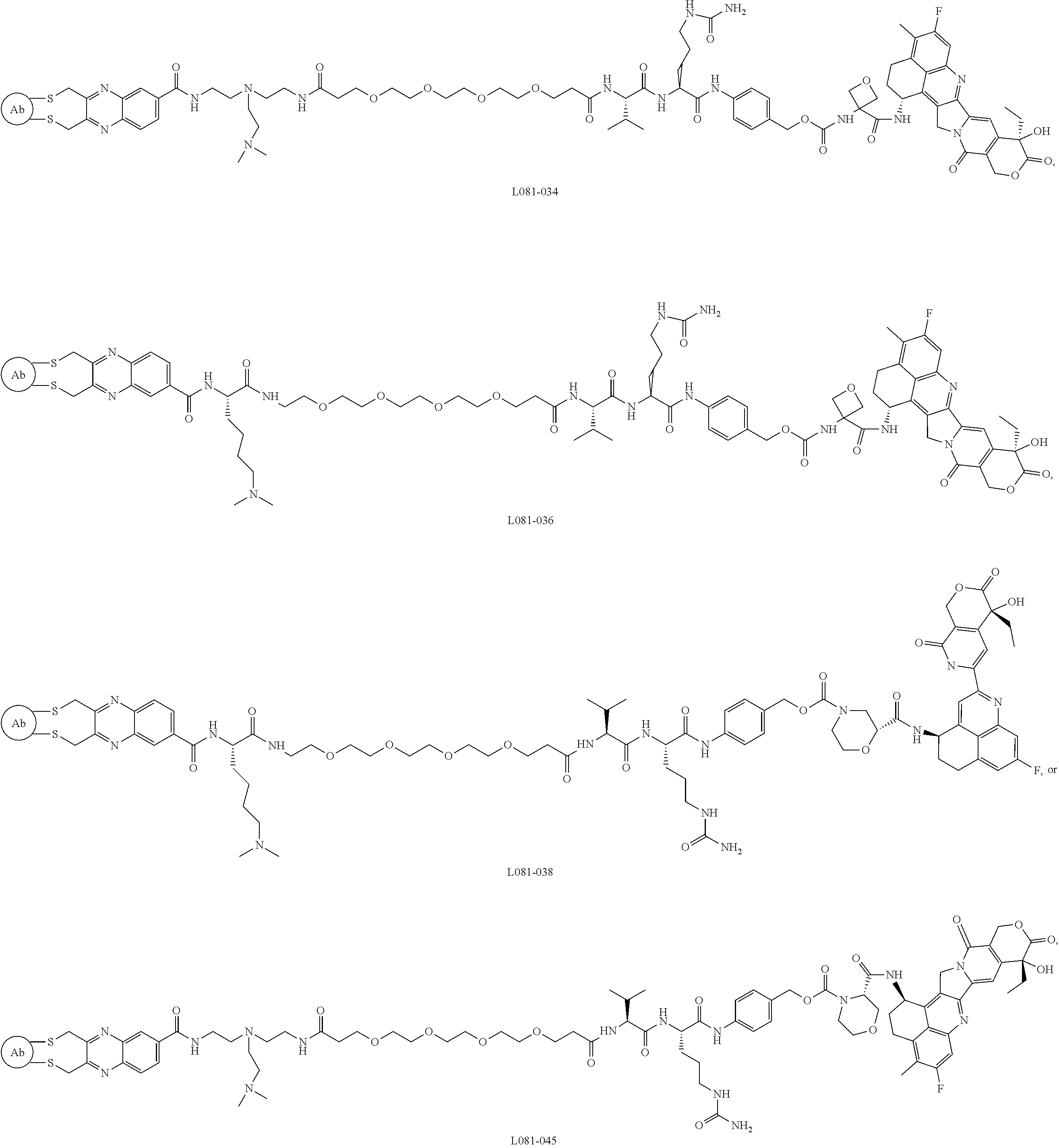

or a pharmaceutically acceptable salt thereof.

### Precursors

In an aspect, provided herein is a compound of formula (III):

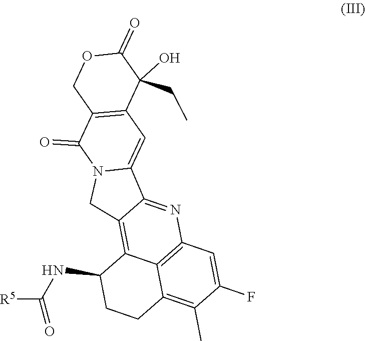

or a pharmaceutically acceptable salt, solvate, hydrate, stereoisomer, or prodrug thereof, wherein R⁵ is a substituted or unsubstituted heterocycloalkyl or a substituted or unsubstituted heteroaryl.

In embodiments, R⁵ is a substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl). In embodiments, R⁵ is substituted with one or more substituent groups. In embodiments, R⁵ is substituted with one or more size-limited substituent groups. In embodiments, R⁵ is substituted with one or more lower substituent groups.

In embodiments, R⁵ is substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R⁵ is unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is substituted (e.g., substituted with at least one substituent group, size-limited substituent group, or lower substituent group) heteroaryl (e.g., 5 to 10 membered heteroaryl, 5 to 9 membered heteroaryl, or 5 to 6 membered heteroaryl). In embodiments, R⁵ is unsubstituted heteroaryl (e.g., 5 to 10 membered heteroaryl, 5 to 9 membered heteroaryl, or 5 to 6 membered heteroaryl).

In embodiments, R⁵ is substituted (e.g., with a substituent group, a size-limited substituent group or a lower substituent group) or unsubstituted 3 to 8 membered heterocycloalkyl. In embodiments, R⁵ is substituted (e.g., with a substituent group, a size-limited substituent group or a lower substituent group) 3 to 8 membered heterocycloalkyl. In embodiments, R⁵ is unsubstituted 3 to 8 membered heterocycloalkyl.

In embodiments, R⁵ is substituted (e.g., with a substituent group, a size-limited substituent group or a lower substituent group) or unsubstituted 3 to 6 membered heterocycloalkyl. In embodiments, R⁵ is substituted (e.g., with a substituent group, a size-limited substituent group or a lower substituent group) 3 to 6 membered heterocycloalkyl. In embodiments, R⁵ is unsubstituted 3 to 6 membered heterocycloalkyl.

In embodiments, R⁵ is substituted (e.g., with a substituent group, a size-limited substituent group or a lower substituent group) or unsubstituted heterocyclobutyl, heterocyclopentyl or heterocyclohexyl. In embodiments, R⁵ is substituted (e.g., with a substituent group, a size-limited substituent group or a lower substituent group) heterocyclobutyl, heterocyclopentyl or heterocyclohexyl. In embodiments, R⁵ is unsubstituted heterocyclobutyl, heterocyclopentyl or heterocyclohexyl.

In embodiments, R⁵ is substituted (e.g., with a substituent group, a size-limited substituent group or a lower substituent group) or unsubstituted heterocyclobutyl. In embodiments, R⁵ is substituted (e.g., with a substituent group, a size-limited substituent group or a lower substituent group) heterocyclobutyl. In embodiments, R⁵ is unsubstituted heterocyclobutyl.

In embodiments, R⁵ is substituted (e.g., with a substituent group, a size-limited substituent group or a lower substituent group) or unsubstituted heterocyclopentyl. In embodiments, R⁵ is substituted (e.g., with a substituent group, a size-limited substituent group or a lower substituent group) heterocyclopentyl. In embodiments, R⁵ is substituted unsubstituted heterocyclopentyl.

In embodiments, R⁵ is substituted (e.g., with a substituent group, a size-limited substituent group or a lower substituent group) or unsubstituted heterocyclohexyl. In embodiments, R⁵ is substituted (e.g., with a substituent group, a size-limited substituent group or a lower substituent group) heterocyclohexyl. In embodiments, R⁵ is unsubstituted heterocyclohexyl.

In embodiments, R⁵ is substituted (e.g., with a substituent group, a size-limited substituent group or a lower substituent group) or unsubstituted 5 to 10 membered heteroaryl. In embodiments, R⁵ is substituted (e.g., with a substituent group, a size-limited substituent group or a lower substituent group) 5 to 10 membered heteroaryl. In embodiments, R⁵ is unsubstituted 5 to 10 membered heteroaryl.

In embodiments, R⁵ is substituted (e.g., with a substituent group, a size-limited substituent group or a lower substituent group) or unsubstituted 5 to 9 membered heteroaryl. In embodiments, R⁵ is substituted (e.g., with a substituent group, a size-limited substituent group or a lower substituent group) 5 to 9 membered heteroaryl. In embodiments, R⁵ is unsubstituted 5 to 9 membered heteroaryl.

In embodiments, R⁵ is substituted (e.g., with a substituent group, a size-limited substituent group or a lower substituent group) or unsubstituted 5 to 6 membered heteroaryl. In embodiments, R⁵ is substituted (e.g., with a substituent group, a size-limited substituent group or a lower substituent group) 5 to 6 membered heteroaryl. In embodiments, R⁵ is unsubstituted 5 to 6 membered heteroaryl.

In embodiments, R⁵ is substituted (e.g., with a substituent group, a size-limited substituent group or a lower substituent group) or unsubstituted furanyl, pyrrolyl, pyridyl, pyranyl, imidazolyl, or thiazolyl. In embodiments, R⁵ is substituted (e.g., with a substituent group, a size-limited substituent group or a lower substituent group) furanyl, pyrrolyl, pyridyl, pyranyl, imidazolyl, or thiazolyl. In embodiments, R⁵ is unsubstituted furanyl, pyrrolyl, pyridyl, pyranyl, imidazolyl, or thiazolyl.

In embodiments, R⁵ is substituted (e.g., with a substituent group, a size-limited substituent group or a lower substituent group) or unsubstituted furanyl. In embodiments, R⁵ is substituted (e.g., with a substituent group, a size-limited substituent group or a lower substituent group) furanyl. In embodiments, R⁵ is unsubstituted furanyl.

In embodiments, R⁵ is substituted (e.g., with a substituent group, a size-limited substituent group or a lower substituent group) or unsubstituted pyrrolyl. In embodiments, R⁵ is substituted (e.g., with a substituent group, a size-limited substituent group or a lower substituent group) pyrrolyl. In embodiments, R⁵ is unsubstituted pyrrolyl.

In embodiments, R⁵ is substituted (e.g., with a substituent group, a size-limited substituent group or a lower substituent group) or unsubstituted pyridyl. In embodiments, R⁵ is substituted (e.g., with a substituent group, a size-limited substituent group or a lower substituent group) pyridyl. In embodiments, R⁵ is unsubstituted pyridyl.

In embodiments, R⁵ is substituted (e.g., with a substituent group, a size-limited substituent group or a lower substituent group) or unsubstituted pyranyl. In embodiments, R⁵ is substituted (e.g., with a substituent group, a size-limited substituent group or a lower substituent group) pyranyl. In embodiments, R⁵ is unsubstituted pyranyl.

In embodiments, R⁵ is substituted (e.g., with a substituent group, a size-limited substituent group or a lower substituent group) or unsubstituted imidazolyl. In embodiments, R⁵ is substituted (e.g., with a substituent group, a size-limited substituent group or a lower substituent group) imidazolyl. In embodiments, R⁵ is unsubstituted imidazolyl.

In embodiments, R⁵ is substituted (e.g., with a substituent group, a size-limited substituent group or a lower substituent group) or unsubstituted thiazolyl. In embodiments, R⁵ is substituted (e.g., with a substituent group, a size-limited substituent group or a lower substituent group) thiazolyl. In embodiments, R⁵ is unsubstituted thiazolyl.

### Drug Loading

Drug loading is represented by m, the average number of drug moieties (i.e., D or D′) per monoclonal antibody in an antibody drug conjugate (ADC) of formula (I) or formula (II) and variations thereof. Drug loading may range from 1 to 20 drug moieties per antibody. The ADCs of formula (I) or formula (II), and any embodiment, variation, or aspect thereof, include collections of antibodies conjugated with a range of drug moieties, from 1 to 20. The average number of drug moieties per antibody in preparations of ADCs from conjugation reactions may be characterized by conventional means such as mass spectroscopy, ELISA assay, and HPLC. The quantitative distribution of ADCs in terms of m may also be determined. In some instances, separation, purification, and characterization of homogeneous ADCs where m is a certain value from ADCs with other drug loadings may be achieved by means such as reverse phase HPLC or electrophoresis. In embodiments, the monoclonal antibody is an anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody. In embodiments, the average number of drug moieties (i.e. D or D′) per anti-HER2 antibody may range from 1 to 20 drug moieties per antibody. In embodiments, the average number of drug moieties (i.e. D or D′) per anti-HER2 antibody may range from 1 to 8 drug moieties per antibody.

For some ADCs, m may be limited by the number of attachment sites on the antibody. For example, where the attachment is a cysteine thiol, as in some of the exemplary embodiments described herein, an antibody may have only one or several cysteine thiol groups, or may have only one or several sufficiently reactive thiol groups through which a linker may be attached. In embodiments, the average drug loading for ADC ranges from 1 to about 8, or from about 3 to about 8. In embodiments, L¹ is capable of forming a covalent bond with the thiol groups of the free cysteine(s) in the IgG antibody.

In embodiments, conjugation methods to derivatize a polypeptide with a payload can be accomplished by forming an amide bond with a lysine side chain. Due to the presence of large number of lysine side chain amines with similar reactivity, this conjugation strategy can produce very complex heterogeneous mixtures. The compositions and methods provided herein provide conjugation through lysine, where, in some embodiments, enhanced selectivity of the lysine can result in a less heterogenous mixture. In embodiments, the average drug loading for ADC ranges from 1 to about 20, from 1 to about 8, or from about 3 to about 8. In embodiments, L¹ is capable of forming a covalent bond with the amine group(s) of the lysine(s) in the IgG antibody.

In embodiments, fewer than the theoretical maximum of drug moieties are conjugated to an antibody during a conjugation reaction. Generally, antibodies do not contain many free and reactive cysteine thiol groups which may be linked to a drug moiety; indeed, most cysteine thiol residues in antibodies exist as disulfide bridges. In embodiments, an antibody may be reduced with a reducing agent such as dithiothreitol (DTT) or tricarbonylethylphosphine (TCEP), under partial or total reducing conditions, to generate reactive cysteine thiol groups. In embodiments, an antibody is subjected to denaturing conditions to reveal reactive nucleophilic groups such as lysine or cysteine.

The loading (drug/antibody ratio or “DAR”) of an ADC may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification. DAR can also be controlled by the reactivity of the groups reacting with the antibody.

It is to be understood that where more than one nucleophilic group reacts with a drug-linker intermediate or linker reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In embodiments, a homogeneous ADC with a single loading value may be isolated from the conjugation mixture by electrophoresis or chromatography.

### Anti-HER2 Antibodies

i. Exemplary Antibodies and Antibody Sequences

In embodiments, the ADC comprises an antibody that binds to HER2. HER2 has been reported to be upregulated, for example, in breast cancer independent of baseline levels of HER2 expression. In embodiments, the ADC compounds described herein comprise an anti-HER2 antibody.

In embodiments, the anti-HER2 antibody provided herein comprises a cysteine. In embodiments, the anti-HER2 antibody is bound to a drug, via linker, through the sulfur of a cysteine residue. In embodiments, the anti-HER2 antibody is bound to a drug, via linker, through the sulfur of two cysteine residues.

In embodiments, the anti-HER2 antibody provided herein comprises a lysine. In embodiments, the anti-HER2 antibody is bound to a drug, via linker, through the amine of a lysine residue. In embodiments, the anti-HER2 antibody is bound to a drug, via linker, through the amine of two lysine residues.

In embodiments, the ADC provided herein comprises an anti-HER2 antibody comprising a light chain variable region and a heavy chain variable region, wherein the light chain variable region comprises a light chain complementarity determining region 1 (CDR1) a light chain CDR2 and a light chain CDR3, and the heavy chain variable region comprises a heavy chain CDR1, a heavy chain CDR2, and a heavy chain CDR3.

In embodiments, the ADC provided herein comprises an anti-HER2 antibody comprising at least one, two, three, four, five, or six CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising at least one CDR selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising at least two CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising at least three CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising at least four CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising at least five CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising at least six CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6.

In embodiments, the ADC comprises an anti-HER2 antibody comprising one CDR selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising two CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising three CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising four CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising five CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-HER2 antibody comprising six CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6.

In embodiments, the anti-HER2 antibody comprises a VL CDR1 comprising the sequence of SEQ ID NO: 1, a VL CDR2 comprising the sequence of SEQ ID NO: 2, a VL CDR3 comprising the sequence of SEQ ID NO: 3, a VH CDR1 comprising the sequence of SEQ ID NO: 4, a VH CDR2 comprising the sequence of SEQ ID NO: 5, and a VH CDR3 comprising the sequence of SEQ ID NO: 6. In embodiments, the anti-HER2 antibody comprises a VL CDR1 comprising the sequence of SEQ ID NO: 1. In embodiments, the anti-HER2 antibody comprises a VL CDR2 comprising the sequence of SEQ ID NO: 2. In embodiments, the anti-HER2 antibody comprises a VL CDR3 comprising the sequence of SEQ ID NO: 3. In embodiments, the anti-HER2 antibody comprises a VH CDR1 comprising the sequence of SEQ ID NO: 4. In embodiments, the anti-HER2 antibody comprises a VH CDR2 comprising the sequence of SEQ ID NO: 5. In embodiments, the anti-HER2 antibody comprises and a VH CDR3 comprising the sequence of SEQ ID NO: 6.

In embodiments, the ADC comprises an anti-HER2 antibody comprising the light chain CDR1 has the amino acid sequence of SEQ ID NO:1, the light chain CDR2 has the amino acid sequence of SEQ ID NO:2, the light chain CDR3 has the amino acid sequence of SEQ ID NO:3, the heavy chain CDR1 has the amino acid sequence of SEQ ID NO:4, the heavy chain CDR2 has the amino acid sequence of SEQ ID NO:5, and the heavy chain CDR3 has the amino acid sequence of SEQ ID NO:6.

In embodiments, the anti-HER2 antibody comprises a VL having a sequence with at least 95%, 96%, 97%, 98%, or 99% identity to SEQ ID NO: 7. In embodiments, the anti-HER2 antibody comprises a VL having the sequence of SEQ ID NO: 7. In embodiments, a VL sequence having at least 95%, 96%, 97%, 98%, or 99% identity to SEQ ID NO: 7 contains substitutions (e.g., conservative substitutions), insertions, or deletions relative to the reference sequence, but an anti-HER2 antibody comprising that sequence retains the ability to bind to HER2. In embodiments, a total of 1 to 10 amino acids have been substituted, inserted and/or deleted in SEQ ID NO: 7. In embodiments, a total of 1 to 5 amino acids have been substituted, inserted and/or deleted in SEQ ID NO: 7. In embodiments, substitutions, insertions, or deletions occur in regions outside the CDRs (i.e., in the FRs). In embodiments, the anti-HER2 antibody comprises the VL sequence of SEQ ID NO: 7, and includes post-translational modifications of that sequence.

In embodiments, the anti-HER2 antibody comprises a VH having a sequence with at least 95%, 96%, 97%, 98%, or 99% identity to SEQ ID NO: 8. In embodiments, the anti-HER2 antibody comprises a VH having the sequence of SEQ ID NO: 8. In embodiments, a VH sequence having at least 95%, 96%, 97%, 98%, or 99% identity to SEQ ID NO: 8 contains substitutions (e.g., conservative substitutions), insertions, or deletions relative to the reference sequence, but an anti-HER2 antibody comprising that sequence retains the ability to bind to HER2. In embodiments, a total of 1 to 10 amino acids have been substituted, inserted and/or deleted in SEQ ID NO: 8. In embodiments, a total of 1 to 5 amino acids have been substituted, inserted and/or deleted in SEQ ID NO: 8. In embodiments, substitutions, insertions, or deletions occur in regions outside the CDRs (i.e., in the FRs). In embodiments, the anti-HER2 antibody comprises the VH sequence of SEQ ID NO: 8, and includes post-translational modifications of that sequence.

In embodiments, the anti-HER2 antibody is an IgG antibody. In embodiments, the anti-HER2 antibody is an IgG1, IgG2, IgG3 or IgG4 antibody. In embodiments, the anti-HER2 antibody is an IgG1 or IgG4 antibody. In embodiments, the anti-HER2 antibody is an IgG1 antibody.

In embodiments, an anti-HER2 antibody binds a human HER2. In embodiments, the human HER2 has the amino acid sequence of SEQ ID NO: 16.

In any of the above embodiments, an anti-HER2 antibody is humanized. In embodiment, an anti-HER2 antibody comprises CDRs as in any of the above embodiments, and further comprises a human acceptor framework, e.g. a human immunoglobulin framework or a human consensus framework. In embodiments, a humanized anti-HER2 antibody comprises (a) a VL CDR1 comprising the sequence of SEQ ID NO: 1; (b) a VL CDR2 comprising the sequence of SEQ ID NO: 2; (c) a VL CDR3 comprising the sequence of SEQ ID NO: 3; (d) a VH CDR1 comprising the sequence of SEQ ID NO: 4; (e) a VH CDR2 comprising the sequence of SEQ ID NO: 5; and (f) a VH CDR3 comprising the sequence of SEQ ID NO: 6.

In embodiments, the anti-HER2 antibody is a monoclonal antibody, including a chimeric, humanized, or human antibody. In one embodiment, an anti-HER2 antibody is an antibody fragment, e.g., a Fv, Fab, Fab′, scFv, diabody, or F(ab′)₂ fragment. In another embodiment, the antibody is a substantially full-length antibody, e.g., an IgG1 antibody or other antibody class or isotype as defined herein.

ii. Antibody Affinity

In embodiments, an anti-HER2 antibody provided herein binds a human HER2 with an affinity of ≤10 nM, or ≤5 nM, or ≤4 nM, or ≤3 nM, or ≤2 nM. In embodiments, an anti-HER2 antibody binds a human HER2 with an affinity of >0.0001 nM, or >0.001 nM, or 0.01 nM. Standard assays known to the skilled artisan can be used to determine binding affinity. For example, whether an anti-HER2 antibody “binds with an affinity of” ≤10 nM, or ≤5 nM, or ≤4 nM, or ≤3 nM, or ≤2 nM, can be determined using standard Scatchard analysis utilizing a non-linear curve fitting program (see, for example, Munson et al., Anal Biochem, 107: 220-239, 1980).

In embodiments, the anti-HER2 antibody provided herein has a dissociation constant (Kd) of ≤1 μM, ≤100 nM, ≤10 nM, ≤1 nM, ≤0.1 nM, ≤0.01 nM, or ≤0.001 nM, and optionally is ≥10⁻¹³ M. (e.g. 10⁻⁸ M or less, e.g. from 10⁻⁸ M to 10⁻¹³ M, e.g., from 10⁻⁹ M to 10⁻¹³M)

iii. Antibody Fragments

In embodiments, the antibody (e.g., anti-HER2 antibody) provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

iv. Chimeric and Humanized Antibodies

In embodiments, the anti-HER2 antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J Biol. Chem. 271:22611-22618 (1996)).

v. Human Antibodies

In embodiments, the anti-HER2 antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

vi. Multispecific Antibodies

In embodiments, the anti-HER2 antibody provided herein is a multispecific antibody, e.g. a bispecific antibody. Multispecific antibodies are monoclonal antibodies that have binding specificities for at least two different sites. In embodiments, one of the binding specificities is for HER2 and the other is for any other antigen. In embodiments, bispecific antibodies may bind to two different epitopes of HER2. Bispecific antibodies may also be used to localize cytotoxic agents to cells which express HER2. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to HER2 as well as another, different antigen.

vii. Antibody Variants

In embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In embodiments, the anti-HER2 antibody provided herein has one or more amino acid substitutions. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1
Exemplary Amino acid substitutions.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Mct, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.
   Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In embodiments, an anti-HER2 antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In embodiments, one or more amino acid modifications may be introduced into the Fc region of an anti-HER2 antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In embodiments, an antibody variant that possesses some but not all effector functions is contemplated, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FecyR binding (hence likely lacking ADCC activity), but retains FcRn binding ability. The primary cells for mediating ADCC, NK cells, express FcγRIII only, whereas monocytes express FecyRI, FecyRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C₃c binding ELISA in WO 2006/029879 and WO 2005/100402. To assess complement activation, a CDC assay may be performed (see, for example, Gazzano-Santoro et al., J Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

viii. Antibody Derivatives

In embodiments, a monoclonal antibody, such as an anti-HER2 antibody, provided herein may be further modified (e.g., derivatized) to contain one or more additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

ix. Recombinant Methods and Compositions

Antibodies may be produced using recombinant methods and compositions, e.g., as described in U.S. Pat. No. 4,816,567. One skilled in the art will be familiar with suitable host cells for antibody expression. Exemplary host cells include eukaryotic cells, e.g. a Chinese Hamster Ovary (CHO) cell or lymphoid cell (e.g., Y0, NS0, Sp20 cell).

For recombinant production of an anti-HER2 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

### Methods of Preparing Antibody-Drug Conjugates

An ADC of formula (I) may be prepared by several routes employing organic chemistry reactions, conditions, and reagents known to those skilled in the art, including: (1) reaction of a nucleophilic group of an antibody with a bivalent linker reagent (L¹) to form Ab-L¹ via a covalent bond, followed by reaction with a drug-linker molecule D-L³ or D-L³-L² and (2) reaction of a nucleophilic group of a drug moiety D with a bivalent linker reagent (L³-L²-L¹ or L³-L¹) to form D-L³-L¹ or D-L³-L²-L¹ via a covalent bond, followed by reaction with a nucleophilic group of an antibody or a reduced antibody. An ADC of formula (II) may be prepared by several routes employing organic chemistry reactions, conditions, and reagents known to those skilled in the art, including: (1) reaction of a nucleophilic group of an antibody with a bivalent linker reagent (L) to form Ab-L¹ via a covalent bond, followed by reaction with a drug-linker molecule R′-D′ or R′-D′-L² and (2) reaction of a nucleophilic group of a drug-linker molecule R¹-D′ with a bivalent linker reagent (L²-L¹ or L¹) to form R¹-D′-L¹ or R¹-D′-L²-L¹ via a covalent bond, followed by reaction with a nucleophilic group of an antibody or a reduced antibody. Several such methods are described by Agarwal et al., (2015), Bioconjugate Chem., 26: 176-192.

In embodiments, an antibody may be reduced with a reducing agent such as dithiothreitol (DTT) or tricarbonylethylphosphine (TCEP), under partial or total reducing conditions, to generate reactive cysteine thiol groups. The inter-chain cysteine residues can then be alkylated for example using maleimide. Alternatively, the inter-chain cysteine residues can undergo bridging alkylation for example using bis sulfone linkers or propargyldibromomaleimide followed by Cu-click ligation. In embodiments, the antibody can be conjugated through lysine amino acid. Such conjugation can be a one-step conjugation or a two-step conjugation. In embodiments, the one-step conjugation entails conjugation of the ε-amino group of lysine residue to the drug-linker molecule (D-L³-L²-L¹ or D-L³-L¹) containing an amine-reactive group via amide bonds. In embodiments, the one-step conjugation entails conjugation of the ε-amino group of lysine residue to the drug-linker molecule (R′-D′-L²-L¹ or R′-D′-L¹) containing an amine-reactive group via amide bonds. In embodiments the amine-reactive group is an activated ester. In embodiments, the antibody can be conjugated via a two-step conjugation. The two-step conjugation entails a first step, where a bi-functional reagent containing both amine and thiol reactive functional groups is reacted with the lysine ε-amino group(s). In the second step, the drug-linker molecule (D-L³-L²-L¹, D-L³-L¹, R′-D′-L²-L¹ or R¹-D′-L¹) is conjugated to the thiol reactive group of the bifunctional reagent. Several examples are provided by Jain et al., (2015), Pharm. Res., 32:3526-3540. In embodiments, the first step may involve the functionalization of the antibody with azide followed by a click chemistry reaction with an alkyne modified linker or drug-linker molecule (D-L³-L²-L¹, D-L³-L¹, R′-D′-L²-L¹ or R′-D′-L¹). In embodiments, the first step may involve the functionalization of the antibody with an alkyne followed by a click chemistry reaction with an azide modified linker or drug-linker molecule (D-L³-L²-L, D-L³-L¹, R′-D′-L²-L¹ or R′-D′-L¹). In embodiments, the first step may involve the functionalization of the antibody with an aldehyde followed by a click chemistry reaction with an alkoxyamine or hydrazine modified linker or drug-linker molecule (D-L³-L²-L, D-L³-L¹, R′-D′-L²-L¹ or R′-D′-L¹). In embodiments, the first step may involve the functionalization of the antibody with a tetrazine followed by a click chemistry reaction with a trans-cyclooctene or cyclopropene modified linker or drug-linker molecule (D-L³-L²-L¹, D-L³-L¹, R′-D′-L²-L¹ or R′-D′-L¹). In embodiments, the first step may involve the functionalization of the antibody with a trans-cyclooctene or cyclopropene followed by a click chemistry reaction with a tetrazine modified linker or drug-linker molecule (D-L³-L²-L¹, D-L³-L¹, R′-D′-L²-L¹ or R′-D′-L¹). Some examples are described by Pickens et al., (2018), Bioconjug. Chem., 29:686-701; Li et al., (2018), Mabs, 10:712-719; and Chio et al., (2020), Methods Mol. Biol., 2078:83-97.

In an aspect, an ADC of formula (I) or formula (II) can be prepared by reacting a monoclonal antibody (Ab) with a molecule of formula (P-I) or formula (P-II):

B-L²-L³-D (P-I)

or

B-L²-D′-R¹ (P-II)

or a pharmaceutically acceptable salt thereof, wherein:
B is a reactive moiety capable of forming a bond with the monoclonal antibody;
L² is a bond, —C(O)—, —NH—, Amino Acid Unit, —(CH₂CH₂O)ₙ—, —(CH₂)ₙ—, —O—, -(4-aminobenzyloxycarbonyl)-, —(C(O)CH₂CH₂NH)—, —(C(O)N(R²)CH₂CH₂N(R³))—, or any combination thereof; wherein n is an integer from 1 to 24;
each R² and R³ is independently H or substituted or unsubstituted alkyl;
L³ is a substituted or unsubstituted heterocycloalkylene, substituted or unsubstituted heteroarylene, substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²;
R¹ is a substituted or unsubstituted heterocycloalkyl or a substituted or unsubstituted heteroaryl;
D is

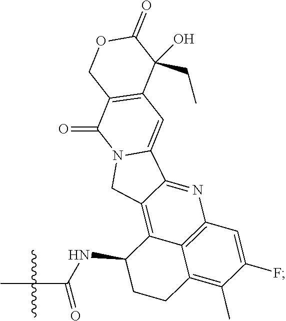 [SMILES: *C(=O)N[C@@H]1CCc2c(C)c(F)cc3nc4c(c1c23)Cn1c-4cc2c(c1=O)COC(=O)[C@@]2(O)CC]

and
D′ is

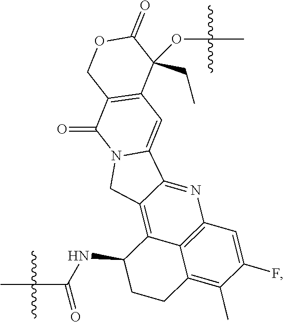

wherein D′ is connected through its amide group to R¹, and through oxygen to L².

In an aspect, an ADC of formula (I) or formula (II) can be prepared by reacting an anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody (Ab) with a molecule of formula (P-I) or formula (P-II):

B-L²-L³-D (P-I)

or

B-L²-D′-R¹ (P-II)

or a pharmaceutically acceptable salt thereof, wherein:
B is a reactive moiety capable of forming a bond with an anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody;
L² is a bond, —C(O)—, —NH—, Amino Acid Unit, —(CH₂CH₂O)ₙ—, —(CH₂)ₙ—, —O—, -(4-aminobenzyloxycarbonyl)-, —(C(O)CH₂CH₂NH)—, —(C(O)N(R²)CH₂CH₂N(R³))—, or any combination thereof, wherein n is an integer from 1 to 24;
each R² and R³ is independently H or substituted or unsubstituted alkyl;
L³ is a substituted or unsubstituted heterocycloalkylene, substituted or unsubstituted heteroarylene, substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²;
R¹ is a substituted or unsubstituted heterocycloalkyl or a substituted or unsubstituted heteroaryl;
D is

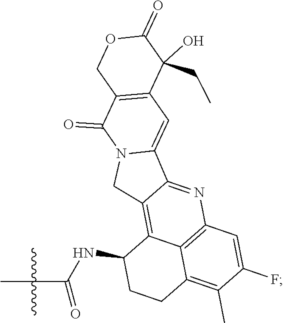 [SMILES: *C(=O)N[C@@H]1CCc2c(C)c(F)cc3nc4c(c1c23)Cn1c-4cc2c(c1=O)COC(=O)[C@@]2(O)CC]

and
D′ is

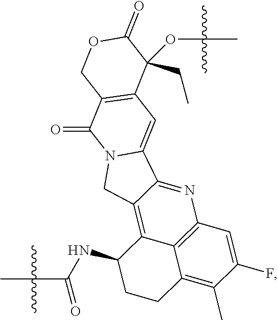

wherein D′ is connected through its amide group to R′, and through oxygen to L².

In embodiments, the monoclonal antibody is modified with a reactive moiety such as an aldehyde, azide, alkyne, tetrazine, hydrazine, alkoxyamine, trans-cyclooctene or cyclopropene. In embodiments, the monoclonal antibody is modified with an aldehyde. In embodiments, the monoclonal antibody is modified with an azide. In embodiments, the monoclonal antibody is modified with a tetrazine. In embodiments, the monoclonal antibody is modified with a alkoxyamine. In embodiments, the monoclonal antibody is modified with a hydrazine. In embodiments, the monoclonal antibody is modified with a trans-cyclooctene. In embodiments, the monoclonal antibody is modified with a cyclopropene.

In embodiments, the monoclonal antibody (Ab) is an anti-HER2 antibody, anti-ROR1 antibody, anti-CD25 antibody, anti-TROP2 antibody, anti-B7-H3 antibody, anti-c-Met antibody, anti-FOLR1 antibody, or anti-CHOP2 antibody. In embodiments, the monoclonal antibody is an anti-HER2 antibody. In embodiments, the monoclonal antibody is an anti-ROR1 antibody. In embodiments, the monoclonal antibody is an anti-CD25 antibody. In embodiments, the monoclonal antibody is an anti-TROP2 antibody. In embodiments, the monoclonal antibody is an anti-B7-H3 antibody. In embodiments, the monoclonal antibody is an anti-c-Met antibody. In embodiments, the monoclonal antibody is an anti-FOLR1 antibody. In embodiments, the monoclonal antibody is an anti-CHOP2 antibody. In embodiments, B is a reactive moiety capable of forming a bond with an anti-HER2 antibody. In embodiments, Ab is a modified anti-HER2 antibody.

In embodiments, Ab is modified with an aldehyde, azide, alkyne, tetrazine, hydrazine, alkoxyamine, trans-cyclooctene or cyclopropene. In embodiments, Ab is modified with an aldehyde. In embodiments, Ab is modified with an azide. In embodiments, Ab is modified with a tetrazine. In embodiments, Ab is modified with a alkoxyamine. In embodiments, Ab is modified with a hydrazine. In embodiments, Ab is modified with a trans-cyclooctene. In embodiments, Ab is modified with a cyclopropene. In embodiments, a modified Ab is a modified anti-HER2 antibody.

In embodiments, n is an integer from 1 to 24. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24.

In embodiments, B is a reactive moiety capable of forming a bond with one or two thiol or amine groups of the anti-HER2 antibody, or with the modified anti-HER2 antibody. In embodiments, the anti-HER2 antibody is modified with an azide, aldehyde, alkyne, tetrazine, hydrazine, alkoxyamine, trans-cyclooctene or cyclopropene.

In embodiments, B is an alkyne, azide, aldehyde, tetrazine, hydrazine, alkoxyamine, trans-cyclooctene, cyclopropene, activated ester, haloacetyl, cycloalkyne, maleimide, or bis-sulfone. In embodiments, B is dibromomaleimide. In embodiments, B is cyclooctyne. In embodiments, the activated ester may be for example pentafluorophenyl ester, tetrafluorophenyl ester, trifluorophenyl ester, difluorophenyl ester, monofluorophenyl or ester, N-hydroxysuccinimide ester.

In embodiments, B is

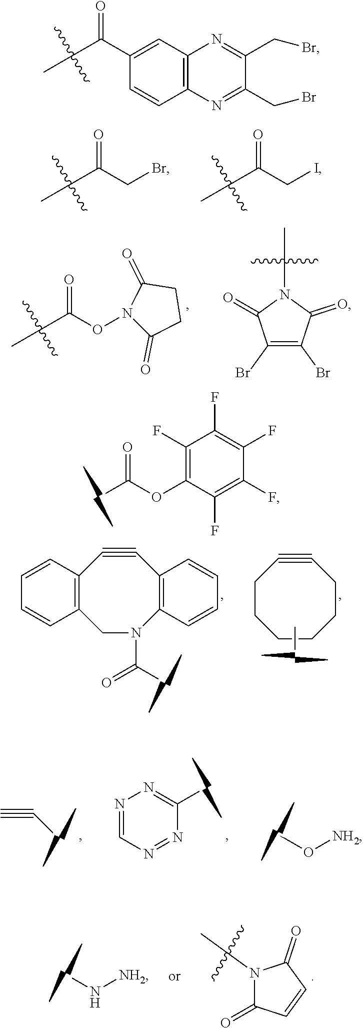

In embodiments, B is

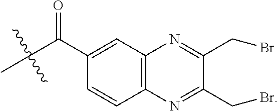 [SMILES: *C(=O)c1ccc2nc(CBr)c(CBr)nc2c1]

In embodiments, B is

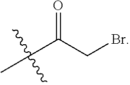 [SMILES: *C(=O)CBr]

In embodiments, B is

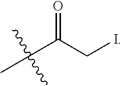 [SMILES: *C(=O)CI]

In embodiments, B is

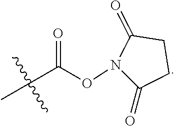 [SMILES: *C(=O)ON1C(=O)CCC1=O]

In embodiments, B is

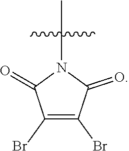 [SMILES: *N1C(=O)C(Br)=C(Br)C1=O]

In embodiments, B is

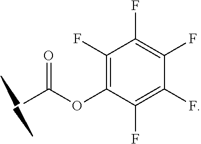

In embodiments, B is

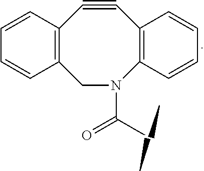

In embodiments, B is

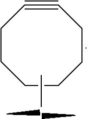

In embodiments, B is

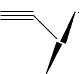

In embodiments, B is

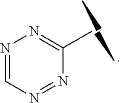

In embodiments, B is

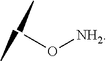

In embodiments, B is

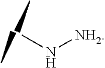

In embodiments, B is

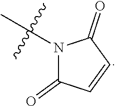 [SMILES: *N1C(=O)C=CC1=O]

In embodiments, B-L²- is

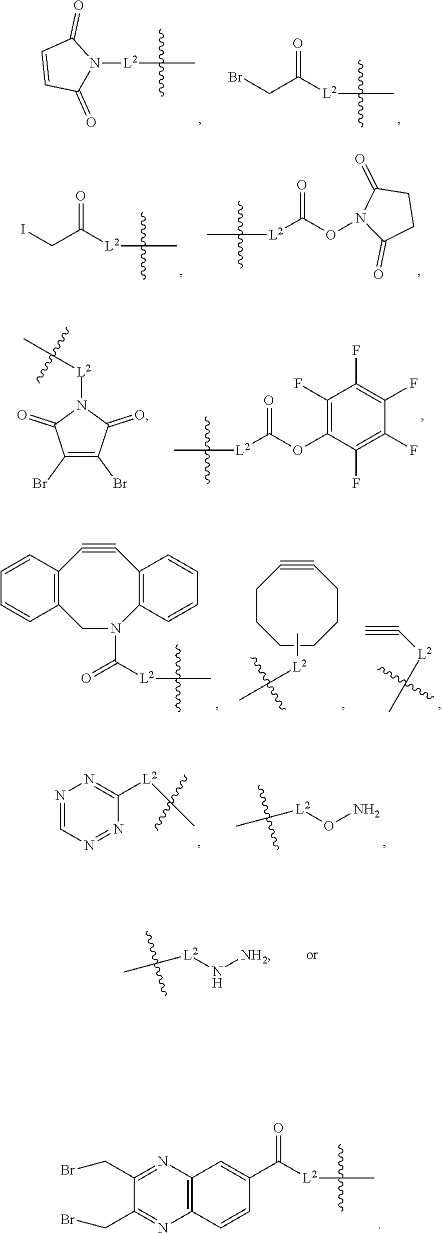

In embodiments, monoclonal antibodies, modified monoclonal antibodies, or anti-HER2 unmodified or modified antibodies (Ab) undergo conjugation reactions with the following reactive B moieties as follows:

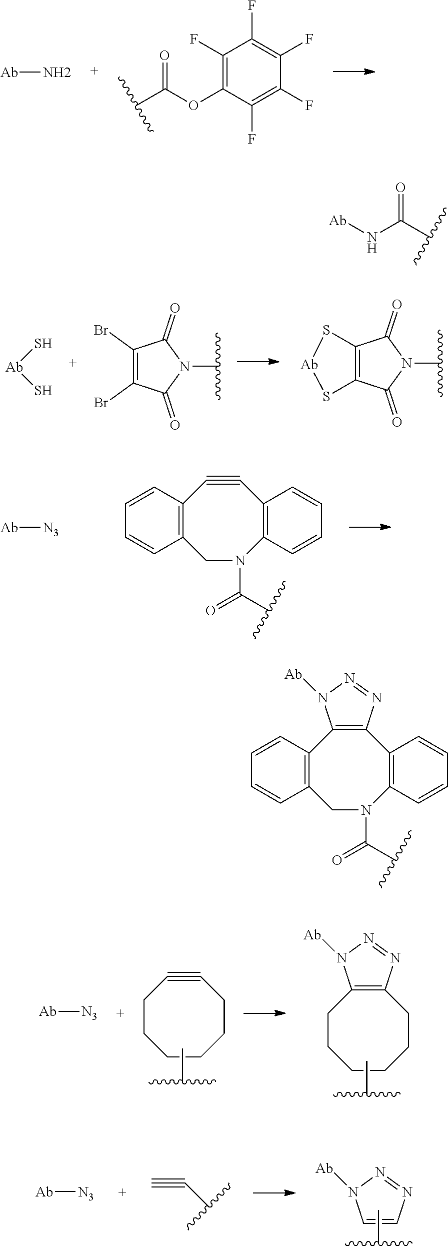

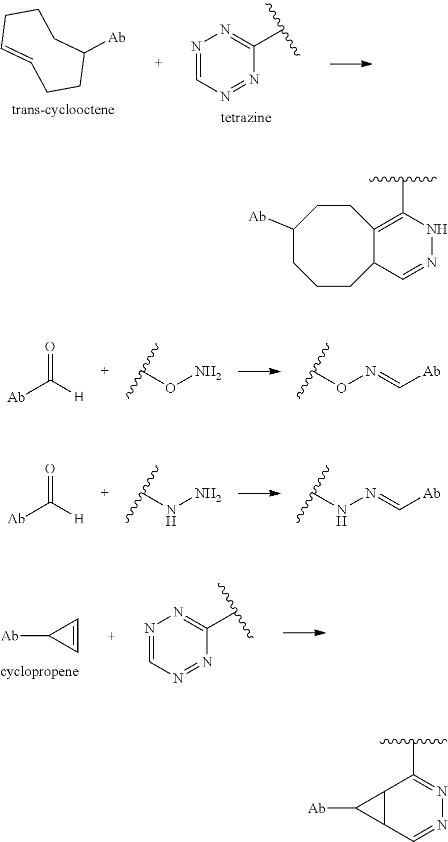

In embodiments, L² is a cleavable or a non-cleavable linker as described in U.S. Pat. Nos. 9,884,127, 9,981,046, 9,801,951, 10,117,944, 10,590,165, and 10,590,165, and US Patent publications Nos. US 2017/0340750, and US 2018/0360985, all of which are incorporated herein in their entireties.

In embodiments, L² is a bond, —C(O)—, —NH—, -Val-, -Phe-, -Lys-, -Gly-, -(4-aminobenzyloxycarbonyl)-, —(C(O)N(R²)CH₂CH₂N(R³))—, -Ser-, -Thr-, -Ala-, -D-Ala-, —O—, -citrulline- (Cit), —(CH₂)ₙ—, —(CH₂CH₂O)ₙ—, or any combination thereof.

In embodiments, each R² and R³ is independently H or substituted or unsubstituted alkyl (e.g., C₁-C₈ alkyl, C₁-C₆ alkyl, or C₁-C₄ alkyl). In embodiments, each R² and R³ is independently H. In embodiments, each R² and R³ is independently substituted or unsubstituted alkyl. In embodiments, each R² and R³ is independently substituted or unsubstituted alkyl (e.g., C₁-C₈ alkyl, C₁-C₆ alkyl, or C₁-C₄ alkyl). In embodiments, each R² and R³ is independently unsubstituted alkyl (e.g., C₁-C₈ alkyl, C₁-C₆ alkyl, or C₁-C₄ alkyl). In embodiments, each R² and R³ is independently substituted alkyl (e.g., C₁-C₈ alkyl, C₁-C₆ alkyl, or C₁-C₄ alkyl).

In embodiments, each R² and R³ is independently H or substituted (e.g., substituted with at least one substituent group, size-limited substituent group, or lower substituent group) or unsubstituted alkyl (e.g., C₁-C₅ alkyl, C₁-C₆ alkyl, or C₁-C₄ alkyl). In embodiments, each R² and R³ is independently substituted (e.g., substituted with at least one substituent group, size-limited substituent group, or lower substituent group) or unsubstituted alkyl. In embodiments, each R² and R³ is independently substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, each R² and R³ is independently unsubstituted alkyl (e.g., C₁-C₈ alkyl, C₁-C₆ alkyl, or C₁-C₄ alkyl). In embodiments, each R² and R³ is independently substituted (e.g., substituted with at least one substituent group, size-limited substituent group, or lower substituent group) alkyl (e.g., C₁-C₈ alkyl, C₁-C₆ alkyl, or C₁-C₄ alkyl).

In embodiments, each R² and R³ is independently methyl, ethyl, propyl, isopropyl, butyl, isobutyl, tert-butyl, pentyl, isopentyl, or hexyl. In embodiments, each R² and R³ is independently methyl. In embodiments, each R² and R³ is independently ethyl. In embodiments, each R² and R³ is independently propyl. In embodiments, each R² and R³ is independently butyl.

In embodiments, L² is a bond, —C(O)—, —NH—, -Val-, -Phe-, -Lys-, -Gly-, -(4-aminobenzyloxycarbonyl)-, —(C(O)N(CH₃)CH₂CH₂N(CH₃))—, -Ser-, -Thr-, -Ala-, -β-Ala-, -citrulline- (Cit), —O—, —(CH₂)ₙ—, —(CH₂CH₂O)ₙ—, or any combination thereof.

In embodiments, L² is —C(O)—, —NH—, -Val-, -Ala-, -Gly-, -Cit-, —O—, -(4-aminobenzyloxycarbonyl)-, —(CH₂)ₙ—, —(CH₂CH₂O)ₙ—, —(C(O)N(CH₃)CH₂CH₂N(CH₃))—, or any combination thereof.

In embodiments, L² is a —C(O)—, —NH—, -Gly-, —(CH₂)ₙ—, —(CH₂CH₂O)ₙ—, or any combination thereof.

In embodiments, L² is a —C(O)—, —NH—, -Val-, -Cit-, -(4-aminobenzyloxycarbonyl)-, —(CH₂)ₙ—, —(CH₂CH₂O)ₙ—, —(C(O)N(CH₃)CH₂CH₂N(CH₃))—, or any combination thereof.

In embodiments, L² is:

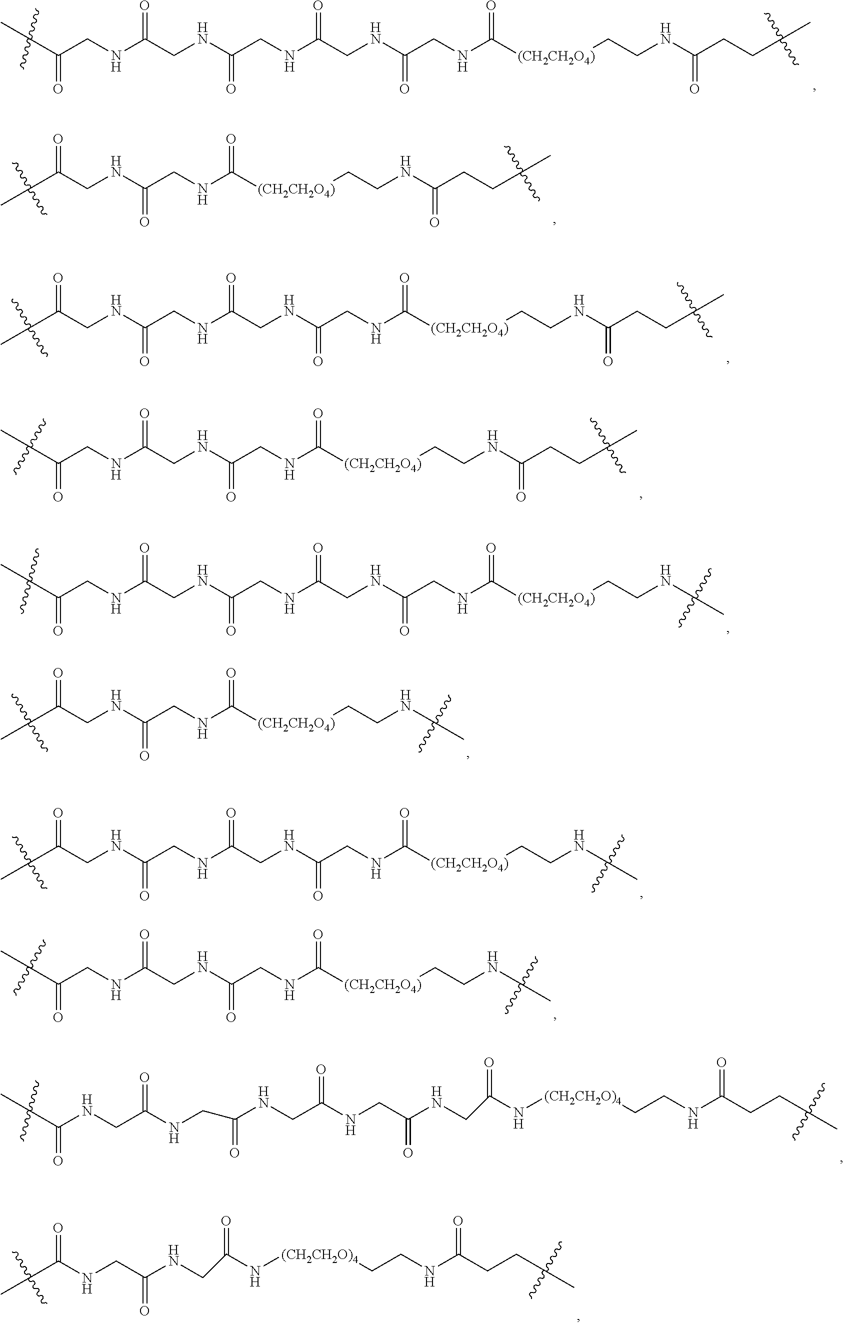

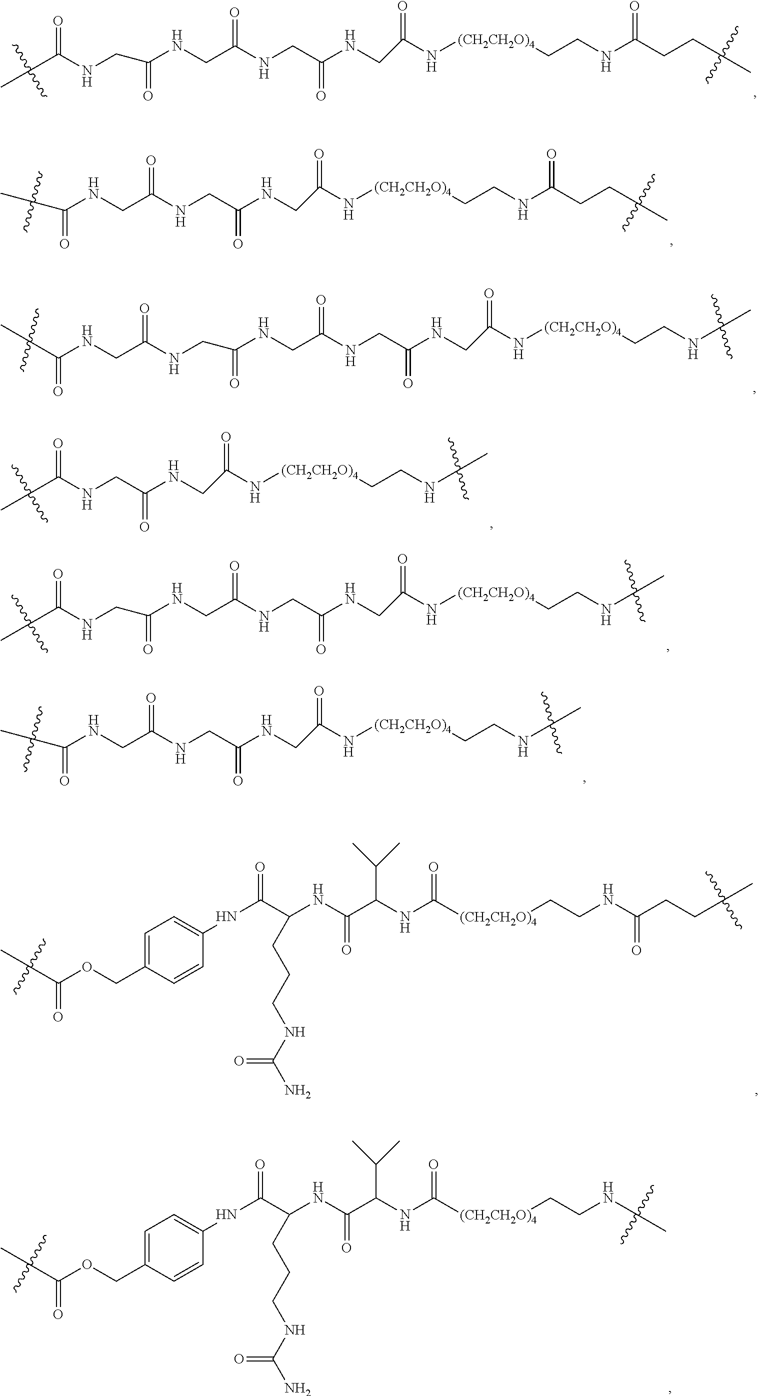

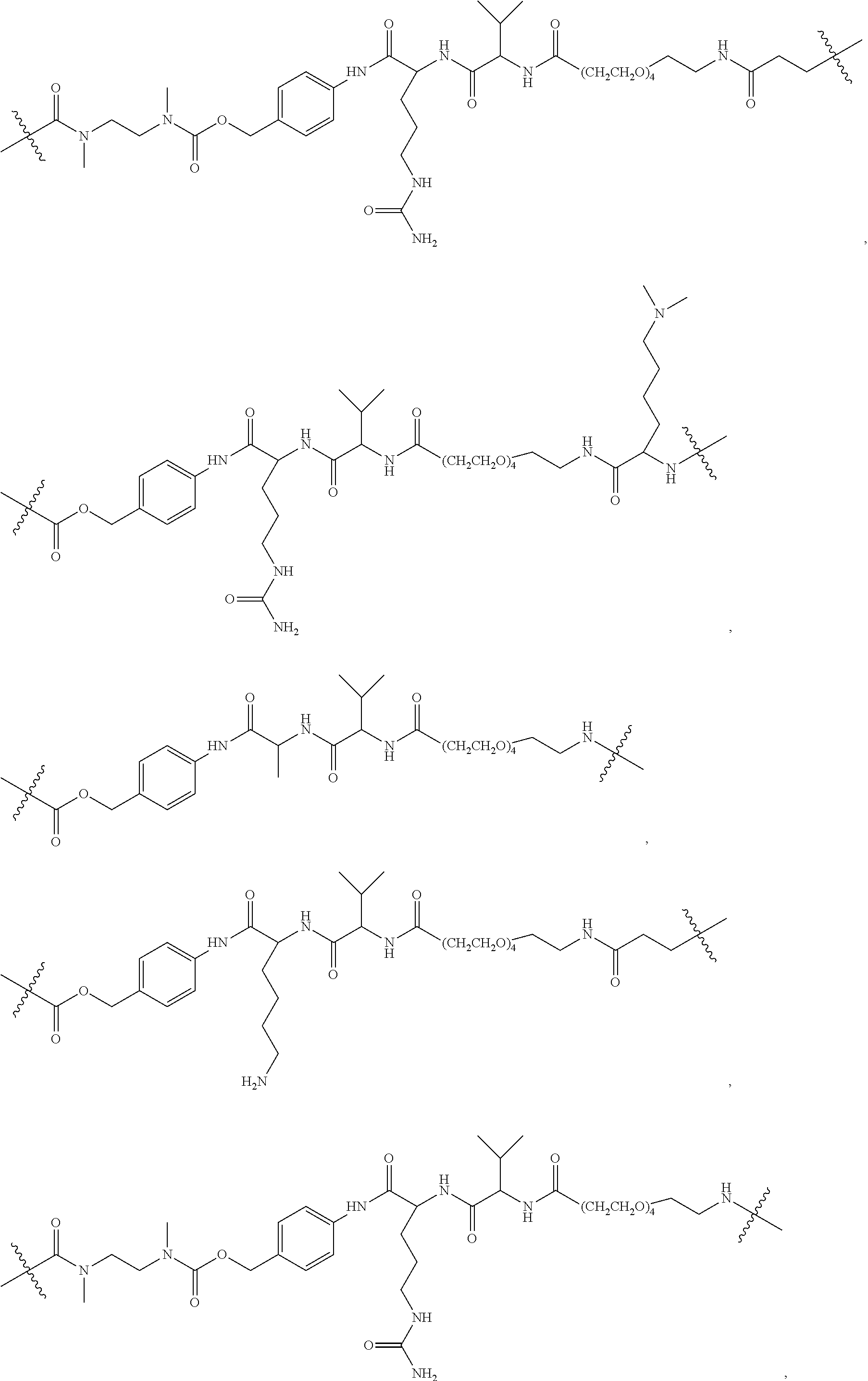

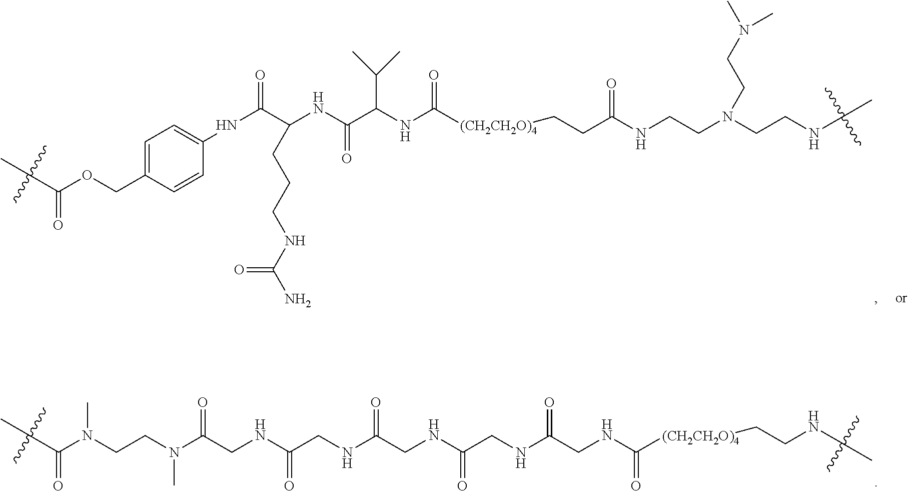

In embodiments, L² is

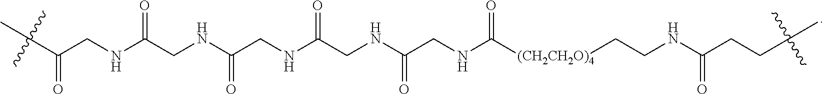

In embodiments, L² is

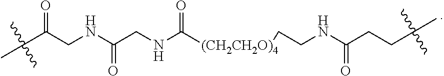

In embodiments, L² is

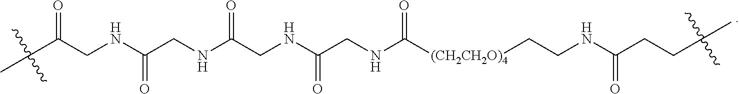

In embodiments, L² is

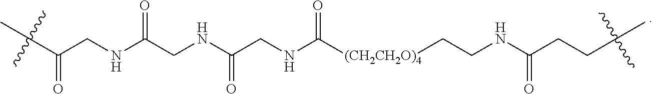

In embodiments, L² is

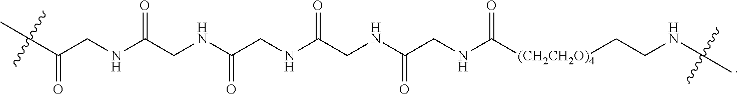

In embodiments, L¹- is

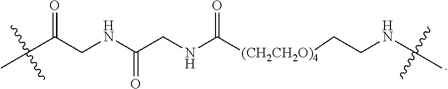

In embodiments, L² is

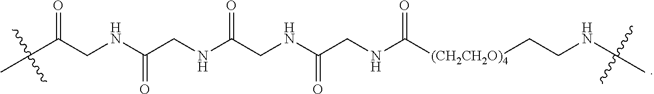

In embodiments, L² is

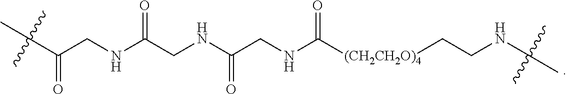

In embodiments, L² is

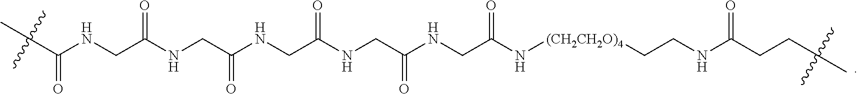

In embodiments, L² is

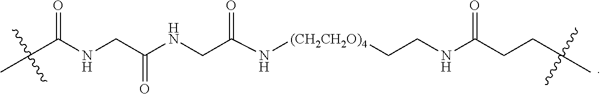

In embodiments, L² is

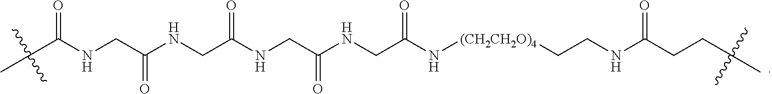

In embodiments, L² is

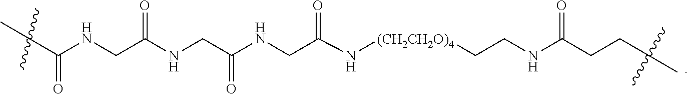

In embodiments, L² is

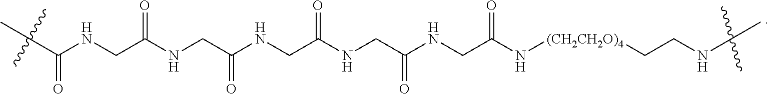

In embodiments, L² is

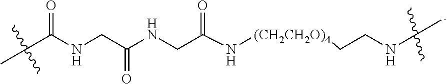

In embodiments, L² is

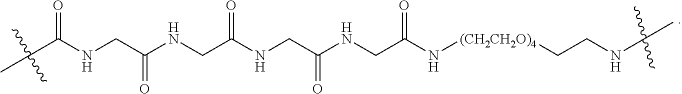

In embodiments, L² is

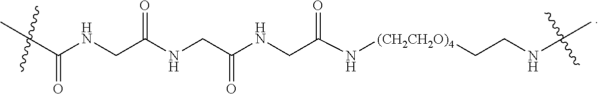

In embodiments, L² is

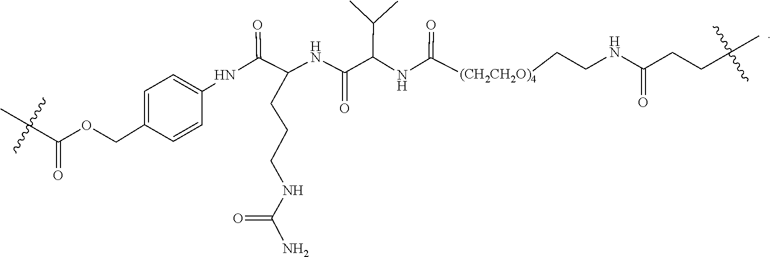

In embodiments, L² is

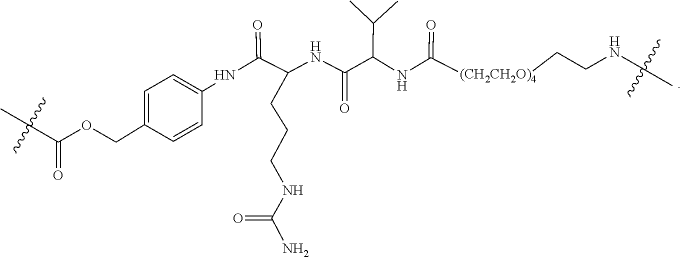

In embodiments, L² is

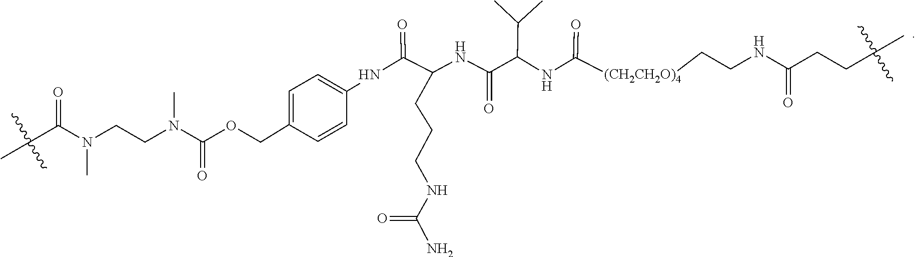

In embodiments, L² is

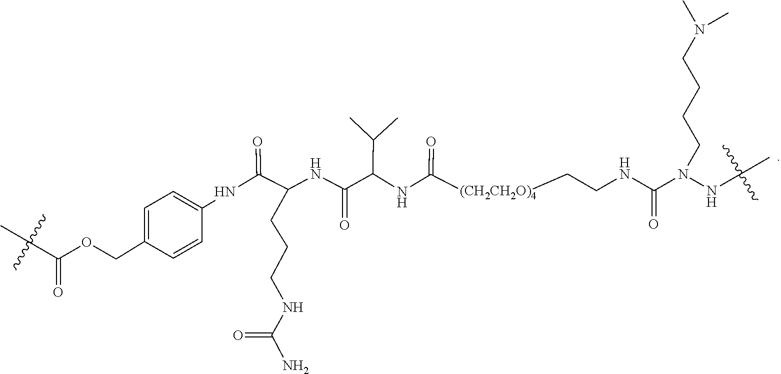

In embodiments, L² is

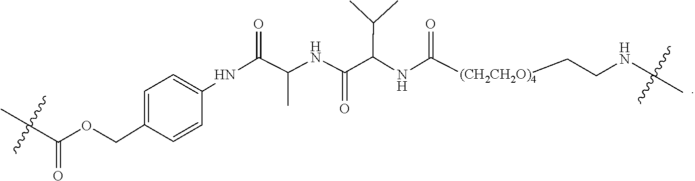

In embodiments, L² is

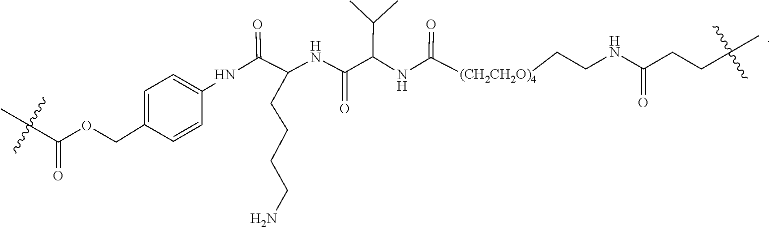

In embodiments, L² is

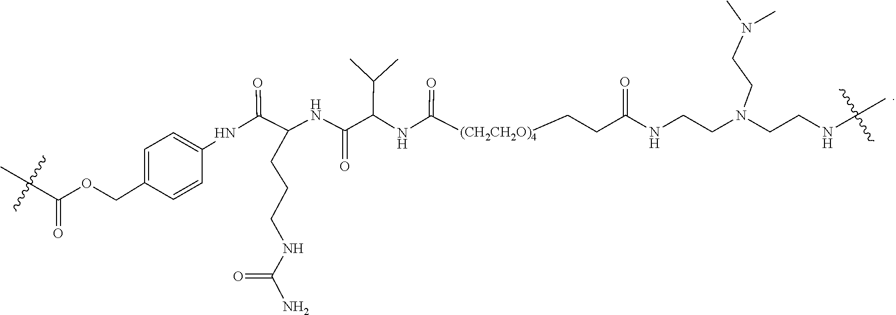

In embodiments, L² is

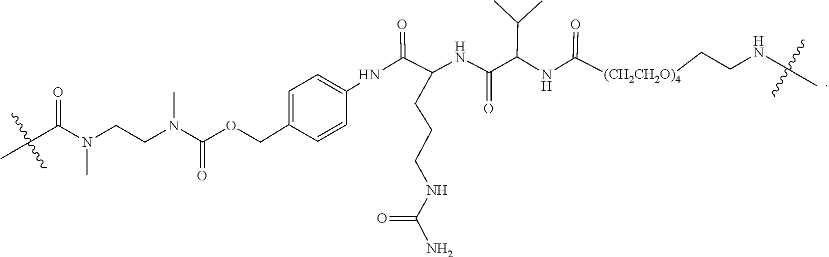

In embodiments, L² is

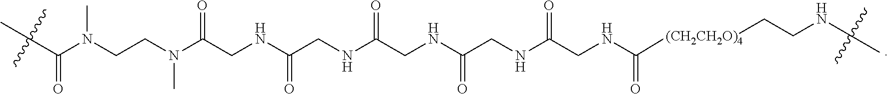

In embodiments, L² is a bond. In embodiments, L² is —C(O)—. In embodiments, L² is —NH—. In embodiments, L² is -Val-. In embodiments, L² is -Phe-. In embodiments, L² is -Lys-. In embodiments, L² is -(4-aminobenzyloxycarbonyl)-. In embodiments, L² is —(CH₂)ₙ—. In embodiments, L² is —(CH₂CH₂O)ₙ—. In embodiments, L² is -Gly-. In embodiments, L² is -Ser-. In embodiments, L² is -Thr-. In embodiments, L² is -Ala-. In embodiments, L² is -β-Ala-. In embodiments, L² is -Cit-. In embodiments, L² is —O—.

In embodiments, L³ is substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene) or substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted —OCH₂-(heterocycloalkyl (e.g., 3 to 8 membered heterocycloalkyl, 3 to 6 membered heterocycloalkyl, or 5 to 6 membered heterocycloalkyl)), substituted (e.g., with a substituent group, a size-limited substituent group or a lower substituent group) or unsubstituted —OCH₂-(heteroaryl (e.g., 5 to 10 membered heteroaryl, 5 to 9 membered heteroaryl, or 5 to 6 membered heteroaryl)), substituted (e.g. with a substituent group, a size-limited substituent group or a lower substituent group) or unsubstituted —CH₂NCH₂-(heterocycloalkyl (e.g., 3 to 8 membered heterocycloalkyl, 3 to 6 membered heterocycloalkyl, or 5 to 6 membered heterocycloalkyl)), or substituted (e.g., with a substituent group, a size-limited substituent group or a lower substituent group) or unsubstituted —CH₂NCH₂-(heteroaryl (e.g., 5 to 10 membered heteroaryl, 5 to 9 membered heteroaryl, or 5 to 6 membered heteroaryl)). In embodiments, L³ is substituted with one or more substituent groups. In embodiments, L³ is substituted with one or more size-limited substituent groups. In embodiments, L³ is substituted with one or more lower substituent groups.

In embodiments, L³ is substituted (e.g., with a substituent group, a size-limited substituent group or a lower substituent group) heterocycloalkylene (e.g., 3 to 8 membered heterocycloalkylene, 3 to 6 membered heterocycloalkylene, or 5 to 6 membered heterocycloalkylene). In embodiments, L³ is unsubstituted heterocycloalkylene (e.g., 3 to 8 membered heterocycloalkylene, 3 to 6 membered heterocycloalkylene, or 5 to 6 membered heterocycloalkylene). In embodiments, L³ is substituted (e.g., with a substituent group, a size-limited substituent group or a lower substituent group) heteroarylene (e.g., 5 to 10 membered heteroarylene, 5 to 9 membered heteroarylene, or 5 to 6 membered heteroarylene). In embodiments, L³ is unsubstituted heteroarylene (e.g., 5 to 10 membered heteroarylene, 5 to 9 membered heteroarylene, or 5 to 6 membered heteroarylene). In embodiments, L³ is substituted (e.g., with a substituent group, a size-limited substituent group, or a lower substituent group) heterocycloalkyl (e.g., 3 to 8 membered heterocycloalkyl, 3 to 6 membered heterocycloalkyl, or 5 to 6 membered heterocycloalkyl). In embodiments, L³ is unsubstituted heterocycloalkyl (e.g., 3 to 8 membered heterocycloalkyl, 3 to 6 membered heterocycloalkyl, or 5 to 6 membered heterocycloalkyl). In embodiments, L³ is substituted (e.g., with a substituent group, a size-limited substituent group, or a lower substituent group) heteroaryl (e.g., 5 to 10 membered heteroaryl, 5 to 9 membered heteroaryl, or 5 to 6 membered heteroaryl). In embodiments, L³ is unsubstituted heteroaryl (e.g., 5 to 10 membered heteroaryl, 5 to 9 membered heteroaryl, or 5 to 6 membered heteroaryl). In embodiments, L³ is substituted (e.g., with a substituent group, a size-limited substituent group, or a lower substituent group) —OCH₂-(heterocycloalkyl (e.g., 3 to 8 membered heterocycloalkyl, 3 to 6 membered heterocycloalkyl, or 5 to 6 membered heterocycloalkyl)). In embodiments, L³ is unsubstituted —OCH₂-(heterocycloalkyl (e.g., 3 to 8 membered heterocycloalkyl, 3 to 6 membered heterocycloalkyl, or 5 to 6 membered heterocycloalkyl)). In embodiments, L³ is substituted (e.g., with a substituent group, a size-limited substituent group, or a lower substituent group) —OCH₂-(heteroaryl (e.g., 5 to 10 membered heteroaryl, 5 to 9 membered heteroaryl, or 5 to 6 membered heteroaryl)). In embodiments, L³ is unsubstituted —OCH₂-(heteroaryl (e.g., 5 to 10 membered heteroaryl, 5 to 9 membered heteroaryl, or 5 to 6 membered heteroaryl)). In embodiments, L³ is substituted (e.g., with a substituent group, a size-limited substituent group, or a lower substituent group) —CH₂NCH₂-(heterocycloalkyl (e.g., 3 to 8 membered heterocycloalkyl, 3 to 6 membered heterocycloalkyl, or 5 to 6 membered heterocycloalkyl)). In embodiments, L³ is unsubstituted —CH₂NCH₂-(heterocycloalkyl (e.g., 3 to 8 membered heterocycloalkyl, 3 to 6 membered heterocycloalkyl, or 5 to 6 membered heterocycloalkyl)). In embodiments, L³ is substituted (e.g., with a substituent group, a size-limited substituent group, or a lower substituent group) —CH₂NCH₂-(heteroaryl (e.g., 5 to 10 membered heteroaryl, 5 to 9 membered heteroaryl, or 5 to 6 membered heteroaryl)). In embodiments, L³ is unsubstituted —CH₂NCH₂-(heteroaryl (e.g., 5 to 10 membered heteroaryl, 5 to 9 membered heteroaryl, or 5 to 6 membered heteroaryl)).

In embodiments, L³ is substituted (e.g., with a substituent group, a size-limited substituent group or a lower substituent group) or unsubstituted 3 to 8 membered heterocycloalkylene. In embodiments, L³ is substituted (e.g., with a substituent group, a size-limited substituent group or a lower substituent group) 3 to 8 membered heterocycloalkylene. In embodiments, L³ is unsubstituted 3 to 8 membered heterocycloalkylene. In embodiments, L³ is substituted (e.g., with a substituent group, a size-limited substituent group, or a lower substituent group) or unsubstituted 3 to 8 membered heterocycloalkyl. In embodiments, L³ is substituted (e.g., with a substituent group, a size-limited substituent group, or a lower substituent group) 3 to 8 membered heterocycloalkyl. In embodiments, L³ is unsubstituted 3 to 8 membered heterocycloalkyl. In embodiments, L³ is substituted (e.g., with a substituent group, a size-limited substituent group, or a lower substituent group) or unsubstituted —CH₂NCH₂-(3 to 8 membered heterocycloalkyl). In embodiments, L³ is substituted (e.g., with a substituent group, a size-limited substituent group, or a lower substituent group) —CH₂NCH₂-(3 to 8 membered heterocycloalkyl). In embodiments, L³ is unsubstituted —CH₂NCH₂-(3 to 8 membered heterocycloalkyl). In embodiments, L³ is substituted (e.g., with a substituent group, a size-limited substituent group, or a lower substituent group) or unsubstituted —OCH₂-(3 to 8 membered heterocycloalkyl). In embodiments, L³ is substituted (e.g., with a substituent group, a size-limited substituent group, or a lower substituent group) —OCH₂-(3 to 8 membered heterocycloalkyl). In embodiments, L³ is unsubstituted —OCH₂-(3 to 8 membered heterocycloalkyl).

In embodiments, L³ is substituted (e.g., with a substituent group, a size-limited substituent group or a lower substituent group) or unsubstituted 3 to 6 membered heterocycloalkylene. In embodiments, L³ is substituted (e.g., with a substituent group, a size-limited substituent group or a lower substituent group) 3 to 6 membered heterocycloalkylene. In embodiments, L³ is unsubstituted 3 to 6 membered heterocycloalkylene. In embodiments, L³ is substituted (e.g., with a substituent group, a size-limited substituent group, or a lower substituent group) or unsubstituted 3 to 6 membered heterocycloalkyl. In embodiments, L³ is substituted (e.g., with a substituent group, a size-limited substituent group, or a lower substituent group) 3 to 6 membered heterocycloalkyl. In embodiments, L³ is unsubstituted 3 to 6 membered heterocycloalkyl. In embodiments, L³ is substituted (e.g., with a substituent group, a size-limited substituent group, or a lower substituent group) or unsubstituted —CH₂NCH₂-(3 to 6 membered heterocycloalkyl). In embodiments, L³ is substituted (e.g., with a substituent group, a size-limited substituent group, or a lower substituent group) —CH₂NCH₂-(3 to 6 membered heterocycloalkyl). In embodiments, L³ is unsubstituted —CH₂NCH₂-(3 to 6 membered heterocycloalkyl). In embodiments, L³ is substituted (e.g., with a substituent group, a size-limited substituent group, or a lower substituent group) or unsubstituted —OCH₂-(3 to 6 membered heterocycloalkyl). In embodiments, L³ is substituted (e.g., with a substituent group, a size-limited substituent group, or a lower substituent group) —OCH₂-(3 to 6 membered heterocycloalkyl). In embodiments, L³ is unsubstituted —OCH₂-(3 to 6 membered heterocycloalkyl).

In embodiments, L³ is substituted (e.g., with a substituent group, a size-limited substituent group or a lower substituent group) or unsubstituted heterocyclobutylene, heterocyclopentylene or heterocyclohexylene. In embodiments, L³ is substituted (e.g., with a substituent group, a size-limited substituent group or a lower substituent group) heterocyclobutylene, heterocyclopentylene or heterocyclohexylene. In embodiments, L³ is unsubstituted heterocyclobutylene, heterocyclopentylene or heterocyclohexylene. In embodiments, L³ is substituted (e.g., with a substituent group, a size-limited substituent group or a lower substituent group) or unsubstituted heterocyclobutyl, heterocyclopentyl or heterocyclohexyl. In embodiments, L³ is substituted (e.g., with a substituent group, a size-limited substituent group, or a lower substituent group) heterocyclobutyl, heterocyclopentyl or heterocyclohexyl. In embodiments, L³ is unsubstituted heterocyclobutyl, heterocyclopentyl or heterocyclohexyl. In embodiments, L³ is substituted (e.g., with a substituent group, a size-limited substituent group or a lower substituent group) or unsubstituted —CH₂NCH₂-(heterocyclobutyl, heterocyclopentyl, or heterocyclohexyl). In embodiments, L³ is substituted (e.g., with a substituent group, a size-limited substituent group or a lower substituent group) —CH₂NCH₂-(heterocyclobutyl, heterocyclopentyl, or heterocyclohexyl). In embodiments, L³ is unsubstituted —CH₂NCH₂-(heterocyclobutyl, heterocyclopentyl, or heterocyclohexyl). In embodiments, L³ is substituted (e.g., with a substituent group, a size-limited substituent group or a lower substituent group) or unsubstituted —OCH₂-(heterocyclobutyl, heterocyclopentyl, or heterocyclohexyl). In embodiments, L³ is substituted (e.g., with a substituent group, a size-limited substituent group or a lower substituent group) —OCH₂-(heterocyclobutyl, heterocyclopentyl, or heterocyclohexyl). In embodiments, L³ is unsubstituted —OCH₂-(heterocyclobutyl, heterocyclopentyl, or heterocyclohexyl).

In embodiments, L³ is substituted (e.g., with a substituent group, a size-limited substituent group or a lower substituent group) or unsubstituted heterocyclobutylene. In embodiments, L³ is substituted (e.g., with a substituent group, a size-limited substituent group or a lower substituent group) heterocyclobutylene. In embodiments, L³ is unsubstituted heterocyclobutylene. In embodiments, L³ is substituted (e.g., with a substituent group, a size-limited substituent group or a lower substituent group) or unsubstituted heterocyclobutyl. In embodiments, L³ is substituted (e.g., with a substituent group, a size-limited substituent group or a lower substituent group) heterocyclobutyl. In embodiments, L³ is unsubstituted heterocyclobutyl. In embodiments, L³ is substituted (e.g., with a substituent group, a size-limited substituent group or a lower substituent group) or unsubstituted —CH₂NCH₂-(heterocyclobutyl). In embodiments, L³ is substituted (e.g., with a substituent group, a size-limited substituent group or a lower substituent group) —CH₂NCH₂-(heterocyclobutyl). In embodiments, L³ is unsubstituted —CH₂NCH₂-(heterocyclobutyl). In embodiments, L³ is substituted (e.g., with a substituent group, a size-limited substituent group or a lower substituent group) or unsubstituted —OCH₂-(heterocyclobutyl). In embodiments, L³ is substituted (e.g., with a substituent group, a size-limited substituent group or a lower substituent group) —OCH₂-(heterocyclobutyl). In embodiments, L³ is unsubstituted —OCH₂-(heterocyclobutyl).

In embodiments, L³ is substituted (e.g., with a substituent group, a size-limited substituent group or a lower substituent group) or unsubstituted heterocyclopentylene. In embodiments, L³ is substituted (e.g., with a substituent group, a size-limited substituent group or a lower substituent group) heterocyclopentylene. In embodiments, L³ is unsubstituted heterocyclopentylene. In embodiments, L³ is substituted (e.g., with a substituent group, a size-limited substituent group or a lower substituent group) or unsubstituted heterocyclopentyl. In embodiments, L³ is substituted (e.g., with a substituent group, a size-limited substituent group or a lower substituent group) heterocyclopentyl. In embodiments, L³ is unsubstituted heterocyclopentyl. In embodiments, L³ is substituted (e.g., with a substituent group, a size-limited substituent group or a lower substituent group) or unsubstituted —CH₂NCH₂-(heterocyclopentyl). In embodiments, L³ is substituted (e.g., with a substituent group, a size-limited substituent group or a lower substituent group) —CH₂NCH₂-(heterocyclopentyl). In embodiments, L³ is unsubstituted —CH₂NCH₂-(heterocyclopentyl). In embodiments, L³ is substituted (e.g., with a substituent group, a size-limited substituent group or a lower substituent group) or unsubstituted —OCH₂-(heterocyclopentyl). In embodiments, L³ is substituted (e.g., with a substituent group, a size-limited substituent group or a lower substituent group) —OCH₂-(heterocyclopentyl). In embodiments, L³ is unsubstituted —OCH₂-(heterocyclopentyl).

In embodiments, L³ is substituted (e.g., with a substituent group, a size-limited substituent group or a lower substituent group) or unsubstituted heterocyclohexylene. In embodiments, L³ is substituted (e.g., with a substituent group, a size-limited substituent group or a lower substituent group) heterocyclohexylene. In embodiments, L³ is unsubstituted heterocyclohexylene. In embodiments, L³ is substituted (e.g., with a substituent group, a size-limited substituent group or a lower substituent group) or unsubstituted heterocyclohexyl. In embodiments, L³ is substituted (e.g., with a substituent group, a size-limited substituent group or a lower substituent group) heterocyclohexyl. In embodiments, L³ is unsubstituted heterocyclohexyl. In embodiments, L³ is substituted (e.g., with a substituent group, a size-limited substituent group or a lower substituent group) or unsubstituted —CH₂NCH₂-(heterocyclohexyl). In embodiments, L³ is substituted (e.g., with a substituent group, a size-limited substituent group or a lower substituent group) —CH₂NCH₂-(heterocyclohexyl). In embodiments, L³ is unsubstituted —CH₂NCH₂-(heterocyclohexyl). In embodiments, L³ is substituted (e.g., with a substituent group, a size-limited substituent group or a lower substituent group) or unsubstituted —OCH₂-(heterocyclohexyl). In embodiments, L³ is substituted (e.g., with a substituent group, a size-limited substituent group or a lower substituent group) —OCH₂-(heterocyclohexyl). In embodiments, L³ is unsubstituted —OCH₂-(heterocyclohexyl).

In embodiments, L³ is substituted (e.g., with a substituent group, a size-limited substituent group or a lower substituent group) or unsubstituted 5 to 10 membered heteroarylene. In embodiments, L³ is substituted (e.g., with a substituent group, a size-limited substituent group or a lower substituent group) 5 to 10 membered heteroarylene. In embodiments, L³ is unsubstituted 5 to 10 membered heteroarylene. In embodiments, L³ is substituted (e.g., with a substituent group, a size-limited substituent group or a lower substituent group) or unsubstituted 5 to 10 membered heteroaryl. In embodiments, L³ is substituted (e.g., with a substituent group, a size-limited substituent group or a lower substituent group) 5 to 10 membered heteroaryl. In embodiments, L³ is unsubstituted 5 to 10 membered heteroaryl. In embodiments, L³ is substituted (e.g., with a substituent group, a size-limited substituent group or a lower substituent group) or unsubstituted —CH₂NCH₂-(5 to 10 membered heteroaryl). In embodiments, L³ is substituted (e.g., with a substituent group, a size-limited substituent group or a lower substituent group) —CH₂NCH₂-(5 to 10 membered heteroaryl). In embodiments, L³ is unsubstituted —CH₂NCH₂-(5 to 10 membered heteroaryl). In embodiments, L³ is substituted (e.g., with a substituent group, a size-limited substituent group or a lower substituent group) or unsubstituted —OCH₂-(5 to 10 membered heteroaryl). In embodiments, L³ is substituted (e.g., with a substituent group, a size-limited substituent group or a lower substituent group) —OCH₂-(5 to 10 membered heteroaryl). In embodiments, L³ is unsubstituted —OCH₂-(5 to 10 membered heteroaryl).

In embodiments, L³ is substituted (e.g., with a substituent group, a size-limited substituent group or a lower substituent group) or unsubstituted 5 to 9 membered heteroarylene. In embodiments, L³ is substituted (e.g., with a substituent group, a size-limited substituent group or a lower substituent group) 5 to 9 membered heteroarylene. In embodiments, L³ is unsubstituted 5 to 9 membered heteroarylene. In embodiments, L³ is substituted (e.g., with a substituent group, a size-limited substituent group or a lower substituent group) or unsubstituted 5 to 9 membered heteroaryl. In embodiments, L³ is substituted (e.g., with a substituent group, a size-limited substituent group or a lower substituent group) 5 to 9 membered heteroaryl. In embodiments, L³ is unsubstituted 5 to 9 membered heteroaryl. In embodiments, L³ is substituted (e.g., with a substituent group, a size-limited substituent group or a lower substituent group) or unsubstituted —CH₂NCH₂-(5 to 9 membered heteroaryl). In embodiments, L³ is substituted (e.g., with a substituent group, a size-limited substituent group or a lower substituent group) —CH₂NCH₂-(5 to 9 membered heteroaryl). In embodiments, L³ is unsubstituted —CH₂NCH₂-(5 to 9 membered heteroaryl). In embodiments, L³ is substituted (e.g., with a substituent group, a size-limited substituent group or a lower substituent group) or unsubstituted —OCH₂-(5 to 9 membered heteroaryl). In embodiments, L³ is substituted (e.g., with a substituent group, a size-limited substituent group or a lower substituent group) —OCH₂-(5 to 9 membered heteroaryl). In embodiments, L³ is unsubstituted —OCH₂-(5 to 9 membered heteroaryl).

In embodiments, L³ is substituted (e.g., with a substituent group, a size-limited substituent group or a lower substituent group) or unsubstituted 5 to 6 membered heteroarylene. In embodiments, L³ is substituted (e.g., with a substituent group, a size-limited substituent group or a lower substituent group) 5 to 6 membered heteroarylene. In embodiments, L³ is unsubstituted 5 to 6 membered heteroarylene. In embodiments, L³ is substituted (e.g., with a substituent group, a size-limited substituent group or a lower substituent group) or unsubstituted 5 to 6 membered heteroaryl. In embodiments, L³ is substituted (e.g., with a substituent group, a size-limited substituent group or a lower substituent group) 5 to 6 membered heteroaryl. In embodiments, L³ is unsubstituted 5 to 6 membered heteroaryl. In embodiments, L³ is substituted (e.g., with a substituent group, a size-limited substituent group or a lower substituent group) or unsubstituted —CH₂NCH₂-(5 to 6 membered heteroaryl). In embodiments, L³ is substituted (e.g., with a substituent group, a size-limited substituent group or a lower substituent group) —CH₂NCH₂-(5 to 6 membered heteroaryl). In embodiments, L³ is unsubstituted —CH₂NCH₂-(5 to 6 membered heteroaryl). In embodiments, L³ is substituted (e.g., with a substituent group, a size-limited substituent group or a lower substituent group) or unsubstituted —OCH₂-(5 to 6 membered heteroaryl). In embodiments, L³ is substituted (e.g., with a substituent group, a size-limited substituent group or a lower substituent group) —OCH₂-(5 to 6 membered heteroaryl). In embodiments, L³ is unsubstituted —OCH₂-(5 to 6 membered heteroaryl).

In embodiments, L³ is substituted (e.g., with a substituent group, a size-limited substituent group or a lower substituent group) or unsubstituted furanylene, pyrrolylene, pyridylene, pyranylene, imidazolylene, thienylene, oxazolylene, or thiazolylene. In embodiments, L³ is substituted (e.g., with a substituent group, a size-limited substituent group or a lower substituent group) furanylene, pyrrolylene, pyridylene, pyranylene, imidazolylene, thienylene, oxazolylene, or thiazolylene. In embodiments, L³ is unsubstituted furanylene, pyrrolylene, pyridylene, pyranylene, imidazolylene, thienylene, oxazolylene, or thiazolylene. In embodiments, L³ is substituted (e.g., with a substituent group, a size-limited substituent group or a lower substituent group) or unsubstituted furanyl, pyrrolyl, pyridyl, pyranyl, imidazolyl, thienyl, oxazolyl, or thiazolyl. In embodiments, L³ is substituted (e.g., with a substituent group, a size-limited substituent group or a lower substituent group) furanyl, pyrrolyl, pyridyl, pyranyl, imidazolyl, thienyl, oxazolyl, or thiazolyl. In embodiments, L³ is unsubstituted furanyl, pyrrolyl, pyridyl, pyranyl, imidazolyl, thienyl, oxazolyl, or thiazolyl. In embodiments, L³ is substituted (e.g., with a substituent group, a size-limited substituent group or a lower substituent group) or unsubstituted —CH₂NCH₂-(furanyl, pyrrolyl, pyridyl, pyranyl, imidazolyl, thienyl, oxazolyl, or thiazolyl). In embodiments, L³ is substituted (e.g., with a substituent group, a size-limited substituent group or a lower substituent group) —CH₂NCH₂-(furanyl, pyrrolyl, pyridyl, pyranyl, imidazolyl, thienyl, oxazolyl, or thiazolyl). In embodiments, L³ is unsubstituted —CH₂NCH₂-(furanyl, pyrrolyl, pyridyl, pyranyl, imidazolyl, thienyl, oxazolyl, or thiazolyl). In embodiments, L³ is substituted (e.g., with a substituent group, a size-limited substituent group or a lower substituent group) or unsubstituted —OCH₂-(furanyl, pyrrolyl, pyridyl, pyranyl, imidazolyl, thienyl, oxazolyl, or thiazolyl). In embodiments, L³ is substituted (e.g., with a substituent group, a size-limited substituent group or a lower substituent group) —OCH₂-(furanyl, pyrrolyl, pyridyl, pyranyl, imidazolyl, thienyl, oxazolyl, or thiazolyl). In embodiments, L³ is unsubstituted —OCH₂-(furanyl, pyrrolyl, pyridyl, pyranyl, imidazolyl, thienyl, oxazolyl, or thiazolyl).

In embodiments, L³ is substituted (e.g., with a substituent group, a size-limited substituent group or a lower substituent group) or unsubstituted furanylene. In embodiments, L³ is substituted (e.g., with a substituent group, a size-limited substituent group or a lower substituent group) furanylene. In embodiments, L³ is unsubstituted furanylene. In embodiments, L³ is substituted (e.g., with a substituent group, a size-limited substituent group or a lower substituent group) or unsubstituted furanyl. In embodiments, L³ is substituted (e.g., with a substituent group, a size-limited substituent group or a lower substituent group) furanyl. In embodiments, L³ is unsubstituted furanyl. In embodiments, L³ is substituted (e.g., with a substituent group, a size-limited substituent group or a lower substituent group) or unsubstituted —CH₂NCH₂-(furanyl). In embodiments, L³ is substituted (e.g., with a substituent group, a size-limited substituent group or a lower substituent group) —CH₂NCH₂-(furanyl). In embodiments, L³ is unsubstituted —CH₂NCH₂-(furanyl). In embodiments, L³ is substituted (e.g., with a substituent group, a size-limited substituent group or a lower substituent group) or unsubstituted —OCH₂-(furanyl). In embodiments, L³ is substituted (e.g., with a substituent group, a size-limited substituent group or a lower substituent group) —OCH₂-(furanyl). In embodiments, L³ is unsubstituted —OCH₂-(furanyl).

In embodiments, L³ is substituted (e.g., with a substituent group, a size-limited substituent group or a lower substituent group) or unsubstituted pyrrolylene. In embodiments, L³ is substituted (e.g., with a substituent group, a size-limited substituent group or a lower substituent group) pyrrolylene. In embodiments, L³ is unsubstituted pyrrolylene. In embodiments, L³ is substituted (e.g., with a substituent group, a size-limited substituent group or a lower substituent group) or unsubstituted pyrrolyl. In embodiments, L³ is substituted (e.g., with a substituent group, a size-limited substituent group or a lower substituent group) pyrrolyl. In embodiments, L³ is unsubstituted pyrrolyl. In embodiments, L³ is substituted (e.g., with a substituent group, a size-limited substituent group or a lower substituent group) or unsubstituted —CH₂NCH₂-(pyrrolyl). In embodiments, L³ is substituted (e.g., with a substituent group, a size-limited substituent group or a lower substituent group) —CH₂NCH₂-(pyrrolyl). In embodiments, L³ is unsubstituted —CH₂NCH₂-(pyrrolyl). In embodiments, L³ is substituted (e.g., with a substituent group, a size-limited substituent group or a lower substituent group) or unsubstituted —OCH₂-(pyrrolyl). In embodiments, L³ is substituted (e.g., with a substituent group, a size-limited substituent group or a lower substituent group) —OCH₂-(pyrrolyl). In embodiments, L³ is unsubstituted —OCH₂-(pyrrolyl).

In embodiments, L³ is substituted (e.g., with a substituent group, a size-limited substituent group or a lower substituent group) or unsubstituted pyridylene. In embodiments, L³ is substituted (e.g., with a substituent group, a size-limited substituent group or a lower substituent group) pyridylene. In embodiments, L³ is unsubstituted pyridylene. In embodiments, L³ is substituted (e.g., with a substituent group, a size-limited substituent group or a lower substituent group) or unsubstituted pyridyl. In embodiments, L³ is substituted (e.g., with a substituent group, a size-limited substituent group or a lower substituent group) pyridyl. In embodiments, L³ is unsubstituted pyridyl. In embodiments, L³ is substituted (e.g., with a substituent group, a size-limited substituent group or a lower substituent group) or unsubstituted —CH₂NCH₂-(pyridyl). In embodiments, L³ is substituted (e.g., with a substituent group, a size-limited substituent group or a lower substituent group) —CH₂NCH₂-(pyridyl). In embodiments, L³ is unsubstituted —CH₂NCH₂-(pyridyl). In embodiments, L³ is substituted (e.g., with a substituent group, a size-limited substituent group or a lower substituent group) or unsubstituted —OCH₂-(pyridyl). In embodiments, L³ is substituted (e.g., with a substituent group, a size-limited substituent group or a lower substituent group) —OCH₂-(pyridyl). In embodiments, L³ is unsubstituted —OCH₂-(pyridyl).

In embodiments, L³ is substituted (e.g., with a substituent group, a size-limited substituent group or a lower substituent group) or unsubstituted pyranylene. In embodiments, L³ is substituted (e.g., with a substituent group, a size-limited substituent group or a lower substituent group) pyranylene. In embodiments, L³ is unsubstituted pyranylene. In embodiments, L³ is substituted (e.g., with a substituent group, a size-limited substituent group or a lower substituent group) or unsubstituted pyranyl. In embodiments, L³ is substituted (e.g., with a substituent group, a size-limited substituent group or a lower substituent group) pyranyl. In embodiments, L³ is unsubstituted pyranyl. In embodiments, L³ is substituted (e.g., with a substituent group, a size-limited substituent group or a lower substituent group) or unsubstituted —CH₂NCH₂-(pyranyl). In embodiments, L³ is substituted (e.g., with a substituent group, a size-limited substituent group or a lower substituent group) —CH₂NCH₂-(pyranyl). In embodiments, L³ is unsubstituted —CH₂NCH₂-(pyranyl). In embodiments, L³ is substituted (e.g., with a substituent group, a size-limited substituent group or a lower substituent group) or unsubstituted —OCH₂-(pyranyl). In embodiments, L³ is substituted (e.g., with a substituent group, a size-limited substituent group or a lower substituent group) —OCH₂-(pyranyl). In embodiments, L³ is unsubstituted —OCH₂-(pyranyl). In embodiments, L³ is substituted (e.g., with a substituent group, a size-limited substituent group or a lower substituent group) or unsubstituted imidazolylene. In embodiments, L³ is substituted (e.g., with a substituent group, a size-limited substituent group or a lower substituent group) imidazolylene. In embodiments, L³ is unsubstituted imidazolylene. In embodiments, L³ is substituted (e.g., with a substituent group, a size-limited substituent group or a lower substituent group) or unsubstituted imidazolyl. In embodiments, L³ is substituted (e.g., with a substituent group, a size-limited substituent group or a lower substituent group) imidazolyl. In embodiments, L³ is unsubstituted imidazolyl. In embodiments, L³ is substituted (e.g., with a substituent group, a size-limited substituent group or a lower substituent group) or unsubstituted —CH₂NCH₂-(imidazolyl). In embodiments, L³ is substituted (e.g., with a substituent group, a size-limited substituent group or a lower substituent group) —CH₂NCH₂-(imidazolyl). In embodiments, L³ is unsubstituted —CH₂NCH₂-(imidazolyl). In embodiments, L³ is substituted (e.g., with a substituent group, a size-limited substituent group or a lower substituent group) or unsubstituted —OCH₂-(imidazolyl). In embodiments, L³ is substituted (e.g., with a substituent group, a size-limited substituent group or a lower substituent group) —OCH₂-(imidazolyl). In embodiments, L³ is unsubstituted —OCH₂-(imidazolyl). In embodiments, L³ is substituted (e.g., with a substituent group, a size-limited substituent group or a lower substituent group) or unsubstituted thiazolylene. In embodiments, L³ is substituted (e.g., with a substituent group, a size-limited substituent group or a lower substituent group) thiazolylene. In embodiments, L³ is unsubstituted thiazolylene. In embodiments, L³ is substituted (e.g., with a substituent group, a size-limited substituent group or a lower substituent group) or unsubstituted thiazolyl. In embodiments, L³ is substituted (e.g., with a substituent group, a size-limited substituent group or a lower substituent group) thiazolyl. In embodiments, L³ is unsubstituted thiazolyl. In embodiments, L³ is substituted (e.g., with a substituent group, a size-limited substituent group or a lower substituent group) or unsubstituted —CH₂NCH₂-(thiazolyl). In embodiments, L³ is substituted (e.g., with a substituent group, a size-limited substituent group or a lower substituent group) —CH₂NCH₂-(thiazolyl). In embodiments, L³ is unsubstituted —CH₂NCH₂-(thiazolyl). In embodiments, L³ is substituted (e.g., with a substituent group, a size-limited substituent group or a lower substituent group) or unsubstituted —OCH₂-(thiazolyl). In embodiments, L³ is substituted (e.g., with a substituent group, a size-limited substituent group or a lower substituent group) —OCH₂-(thiazolyl). In embodiments, L³ is unsubstituted —OCH₂-(thiazolyl).

In embodiments, L³ is substituted (e.g., with a substituent group, a size-limited substituent group or a lower substituent group) or unsubstituted thienylene. In embodiments, L³ is substituted (e.g., with a substituent group, a size-limited substituent group or a lower substituent group) thienylene. In embodiments, L³ is unsubstituted thienylene. In embodiments, L³ is substituted (e.g., with a substituent group, a size-limited substituent group or a lower substituent group) or unsubstituted thienyl. In embodiments, L³ is substituted (e.g., with a substituent group, a size-limited substituent group or a lower substituent group) thienyl. In embodiments, L³ is unsubstituted thienyl. In embodiments, L³ is substituted (e.g., with a substituent group, a size-limited substituent group or a lower substituent group) or unsubstituted —CH₂NCH₂-(thienyl). In embodiments, L³ is substituted (e.g., with a substituent group, a size-limited substituent group or a lower substituent group) —CH₂NCH₂-(thienyl). In embodiments, L³ is unsubstituted —CH₂NCH₂-(thienyl). In embodiments, L³ is substituted (e.g., with a substituent group, a size-limited substituent group or a lower substituent group) or unsubstituted —OCH₂-(thienyl). In embodiments, L³ is substituted (e.g., with a substituent group, a size-limited substituent group or a lower substituent group) —OCH₂-(thienyl). In embodiments, L³ is unsubstituted —OCH₂-(thienyl).

In embodiments, L³ is substituted (e.g., with a substituent group, a size-limited substituent group or a lower substituent group) or unsubstituted oxazolylene. In embodiments, L³ is substituted (e.g., with a substituent group, a size-limited substituent group or a lower substituent group) oxazolylene. In embodiments, L³ is unsubstituted oxazolylene. In embodiments, L³ is substituted (e.g., with a substituent group, a size-limited substituent group or a lower substituent group) or unsubstituted oxazolyl. In embodiments, L³ is substituted (e.g., with a substituent group, a size-limited substituent group or a lower substituent group) oxazolyl. In embodiments, L³ is unsubstituted oxazolyl. In embodiments, L³ is unsubstituted oxazolylene. In embodiments, L³ is substituted (e.g., with a substituent group, a size-limited substituent group or a lower substituent group) or unsubstituted —CH₂NCH₂-(oxazolyl). In embodiments, L³ is substituted (e.g., with a substituent group, a size-limited substituent group or a lower substituent group) —CH₂NCH₂-(oxazolyl). In embodiments, L³ is unsubstituted —CH₂NCH₂-(oxazolyl). In embodiments, L³ is substituted (e.g., with a substituent group, a size-limited substituent group or a lower substituent group) or unsubstituted —OCH₂-(oxazolyl). In embodiments, L³ is substituted (e.g., with a substituent group, a size-limited substituent group or a lower substituent group) —OCH₂-(oxazolyl). In embodiments, L³ is unsubstituted —OCH₂-(oxazolyl).

In embodiments, R¹ is substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, R¹ is substituted with one or more substituent groups. In embodiments, R¹ is substituted with one or more size-limited substituent groups. In embodiments, R¹ is substituted with one or more lower substituent groups.

In embodiments, R¹ is substituted (e.g., with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R¹ is unsubstituted heterocycloalkyl (e.g., 3 to 8 membered heterocycloalkyl, 3 to 6 membered heterocycloalkyl, or 5 to 6 membered heterocycloalkyl). In embodiments, R¹ is substituted (e.g., with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R¹ is unsubstituted heteroaryl (e.g., 5 to 10 membered heteroaryl, 5 to 9 membered heteroaryl, or 5 to 6 membered heteroaryl).

In embodiments, R¹ is substituted (e.g., with a substituent group, a size-limited substituent group or a lower substituent group) or unsubstituted 3 to 8 membered heterocycloalkyl. In embodiments, R¹ is substituted (e.g., with a substituent group, a size-limited substituent group or a lower substituent group) 3 to 8 membered heterocycloalkyl. In embodiments, R¹ is unsubstituted 3 to 8 membered heterocycloalkyl.

In embodiments, R¹ is substituted (e.g., with a substituent group, a size-limited substituent group or a lower substituent group) or unsubstituted 3 to 6 membered heterocycloalkyl. In embodiments, R¹ is substituted (e.g., with a substituent group, a size-limited substituent group or a lower substituent group) 3 to 6 membered heterocycloalkyl. In embodiments, R¹ is unsubstituted 3 to 6 membered heterocycloalkyl.

In embodiments, R¹ is substituted (e.g., with a substituent group, a size-limited substituent group or a lower substituent group) or unsubstituted heterocyclobutyl, heterocyclopentyl or heterocyclohexyl. In embodiments, R¹ is substituted (e.g., with a substituent group, a size-limited substituent group or a lower substituent group) heterocyclobutyl, heterocyclopentyl or heterocyclohexyl. In embodiments, R′ is unsubstituted heterocyclobutyl, heterocyclopentyl or heterocyclohexyl.

In embodiments, R¹ is substituted (e.g., with a substituent group, a size-limited substituent group or a lower substituent group) or unsubstituted heterocyclobutyl. In embodiments, R¹ is substituted (e.g., with a substituent group, a size-limited substituent group or a lower substituent group) heterocyclobutyl. In embodiments, R¹ is unsubstituted heterocyclobutyl.

In embodiments, R¹ is substituted (e.g., with a substituent group, a size-limited substituent group or a lower substituent group) or unsubstituted heterocyclopentyl. In embodiments, R¹ is substituted (e.g., with a substituent group, a size-limited substituent group or a lower substituent group) heterocyclopentyl. In embodiments, R¹ is unsubstituted heterocyclopentyl.

In embodiments, R¹ is substituted (e.g., with a substituent group, a size-limited substituent group or a lower substituent group) or unsubstituted heterocyclohexyl. In embodiments, R¹ is substituted (e.g., with a substituent group, a size-limited substituent group or a lower substituent group) heterocyclohexyl. In embodiments, R¹ is unsubstituted heterocyclohexyl.

In embodiments, R¹ is substituted (e.g., with a substituent group, a size-limited substituent group or a lower substituent group) or unsubstituted 5 to 10 membered heteroaryl. In embodiments, R¹ is substituted (e.g., with a substituent group, a size-limited substituent group or a lower substituent group) 5 to 10 membered heteroaryl. In embodiments, R¹ is unsubstituted 5 to 10 membered heteroaryl.

In embodiments, R¹ is substituted (e.g., with a substituent group, a size-limited substituent group or a lower substituent group) or unsubstituted 5 to 9 membered heteroaryl. In embodiments, R¹ is substituted (e.g., with a substituent group, a size-limited substituent group or a lower substituent group) 5 to 9 membered heteroaryl. In embodiments, R¹ is unsubstituted 5 to 9 membered heteroaryl.

In embodiments, R¹ is substituted (e.g., with a substituent group, a size-limited substituent group or a lower substituent group) or unsubstituted 5 to 6 membered heteroaryl. In embodiments, R¹ is substituted (e.g., with a substituent group, a size-limited substituent group or a lower substituent group) 5 to 6 membered heteroaryl. In embodiments, R¹ is unsubstituted 5 to 6 membered heteroaryl.

In embodiments, R¹ is substituted (e.g., with a substituent group, a size-limited substituent group or a lower substituent group) or unsubstituted furanyl, pyrrolyl, pyridyl, pyranyl, imidazolyl, or thiazolyl. In embodiments, R¹ is substituted (e.g., with a substituent group, a size-limited substituent group or a lower substituent group) furanyl, pyrrolyl, pyridyl, pyranyl, imidazolyl, or thiazolyl. In embodiments, R¹ is unsubstituted furanyl, pyrrolyl, pyridyl, pyranyl, imidazolyl, or thiazolyl.

In embodiments, R¹ is substituted (e.g., with a substituent group, a size-limited substituent group or a lower substituent group) or unsubstituted furanyl. In embodiments, R¹ is substituted (e.g., with a substituent group, a size-limited substituent group or a lower substituent group) furanyl. In embodiments, R¹ is unsubstituted furanyl.

In embodiments, R¹ is substituted (e.g., with a substituent group, a size-limited substituent group or a lower substituent group) or unsubstituted pyrrolyl. In embodiments, R¹ is substituted (e.g., with a substituent group, a size-limited substituent group or a lower substituent group) pyrrolyl. In embodiments, R¹ is unsubstituted pyrrolyl.

In embodiments, R¹ is substituted (e.g., with a substituent group, a size-limited substituent group or a lower substituent group) or unsubstituted pyridyl. In embodiments, R¹ is substituted (e.g., with a substituent group, a size-limited substituent group or a lower substituent group) pyridyl. In embodiments, R¹ is unsubstituted pyridyl.

In embodiments, R¹ is substituted (e.g., with a substituent group, a size-limited substituent group or a lower substituent group) or unsubstituted pyranyl. In embodiments, R¹ is substituted (e.g., with a substituent group, a size-limited substituent group or a lower substituent group) pyranyl. In embodiments, R¹ is unsubstituted pyranyl.

In embodiments, R¹ is substituted (e.g., with a substituent group, a size-limited substituent group or a lower substituent group) or unsubstituted imidazolyl. In embodiments, R¹ is substituted (e.g., with a substituent group, a size-limited substituent group or a lower substituent group) imidazolyl. In embodiments, R¹ is unsubstituted imidazolyl.

In embodiments, R¹ is substituted (e.g., with a substituent group, a size-limited substituent group or a lower substituent group) or unsubstituted thiazolyl. In embodiments, R¹ is substituted (e.g., with a substituent group, a size-limited substituent group or a lower substituent group) thiazolyl. In embodiments, R¹ is unsubstituted thiazolyl.

In embodiments, an ADC of formula (IA) or formula (IIA) can be prepared by reacting a monoclonal antibody (Ab) with a molecule of formula (P-IA) or formula (P-IIA):

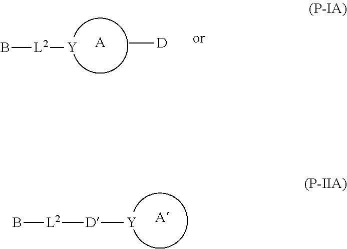

or a pharmaceutically acceptable salt thereof, wherein:
ring A is a substituted or unsubstituted heterocycloalkylene or a substituted or unsubstituted heteroarylene, connected to L² through a heteroatom Y;
ring A′ is a substituted or unsubstituted heterocycloalkyl or a substituted or unsubstituted heteroaryl, connected to D′ through a heteroatom Y;
each Y is independently N, P, or S; and
B, L², D, and D′ are each as defined herein including embodiments.

In embodiments, in formula (P-IA) or (P-IIA),
ring A is a substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene) or substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene). In embodiments, ring A is substituted with one or more substituent groups. In embodiments, ring A is substituted with one or more size-limited substituent groups. In embodiments, ring A is substituted with one or more lower substituent groups. Ring A is connected to L² through a heteroatom Y. In embodiments, each Y is N.

In embodiments, ring A′ is substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, ring A′ is substituted with one or more substituent groups. In embodiments, ring A′ is substituted with one or more size-limited substituent groups. In embodiments, ring A′ is substituted with one or more lower substituent groups. Ring A′ is connected to D′ through a heteroatom Y.

In embodiments, each Y is N.

In embodiments, in formula (P-IA) or (P-IIA),
ring A is a substituted with one or more (e.g., with a substituent group, a size-limited substituent group or a lower substituent group) 3 to 8 membered heterocycloalkylene. In embodiments, ring A is connected to L² through a heteroatom Y. In embodiments, each Y is N.

In embodiments, ring A′ is a substituted with one or more (e.g., with a substituent group, a size-limited substituent group or a lower substituent group) 3 to 8 membered heterocycloalkyl. ring A′ is connected to D′ through a heteroatom Y.

In embodiments, each Y is N.

In embodiments, in formula (P-IA) or (P-IIA),
ring A is a substituted with one or more (e.g., with a substituent group, a size-limited substituent group or a lower substituent group) 5 to 6 membered heterocycloalkylene. Ring A is connected to L² through a heteroatom Y. In embodiments, each Y is N.

In embodiments, ring A′ is a substituted with one or more (e.g., with a substituent group, a size-limited substituent group or a lower substituent group) 5 to 6 membered heterocycloalkyl. ring A′ is connected to D′ through a heteroatom Y.

In embodiments, each Y is N.

In embodiments, an ADC of formula (IB) or formula (IIB) can be prepared by reacting a monoclonal antibody (Ab) with a molecule of formula (P-IB) or formula (P-IIB):

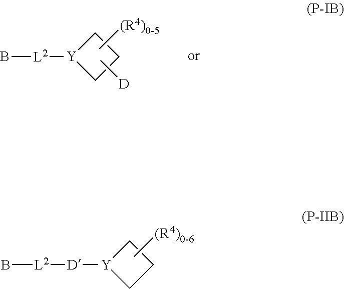

or a pharmaceutically acceptable salt thereof, wherein:
each R⁴ is independently H, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A}, —POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   any two R⁴ substituents on adjacent carbon atoms may optionally be joined to form a substituted or unsubstituted cycloalkyl, substituted or unsubstituted heterocycloalkyl, substituted or unsubstituted aryl or substituted or unsubstituted heteroaryl;
each R^{4A} and R^{4B} is independently H, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X is —Cl, —Br, —I or —F;
each n4 is independently an integer from 0 to 4;
each v4 is independently 1 or 2;
each m4 is independently an integer from 0 to 3; and
each r4 is independently 1 or 2; and
Y, D, D′, B, and L² are each as defined herein including embodiments.

In embodiments, each R⁴ is independently H, halogen, or substituted or unsubstituted alkyl. In embodiments, each R⁴ is independently H, chloro, bromo, iodo, fluoro, or substituted or unsubstituted alkyl. In embodiments, each R⁴ is independently H, chloro, bromo, iodo, fluoro, methyl, ethyl, propyl, isopropyl, butyl, isobutyl, tert-butyl, pentyl, isopentyl, or hexyl. In embodiments, each R⁴ is independently H. In embodiments, each R⁴ is independently fluoro. In embodiments, each R⁴ is independently methyl. In embodiments, each R⁴ is independently ethyl.

In embodiments, an ADC of formula (IC) or formula (IIC) can be prepared by reacting a monoclonal antibody (Ab) with a molecule of formula (P-IC) or formula (P-IIC):

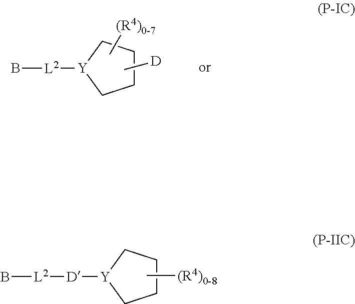

or a pharmaceutically acceptable salt thereof, wherein D, D′, Y, B, L², and R⁴ are each as defined herein including embodiments.

In embodiments, an ADC of formula (ID) or formula (IID) can be prepared by reacting a monoclonal antibody (Ab) with a molecule of formula (P-ID) or formula (P-IID):

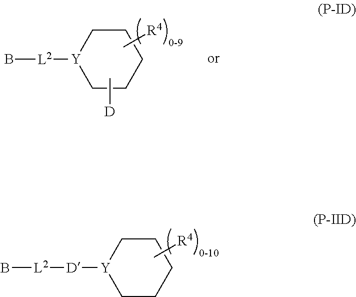

or a pharmaceutically acceptable salt thereof, wherein D, D′, Y, B, L², and R⁴ are each as defined herein including embodiments.

In embodiments, an ADC of formula (ID1) or formula (IID1) can be prepared by reacting a monoclonal antibody (Ab) with a molecule of formula (P-ID1) or formula (P-IID1):

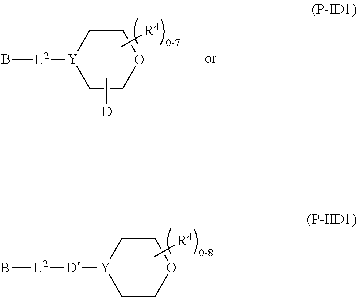

or a pharmaceutically acceptable salt thereof, wherein D, D′, Y, B, L², and R⁴ are each as defined herein including embodiments.

In embodiments, an ADC of formula (IE) or formula (IIE) can be prepared by reacting a monoclonal antibody (Ab) with a molecule of formula (P-IE) or formula (P-IIE):

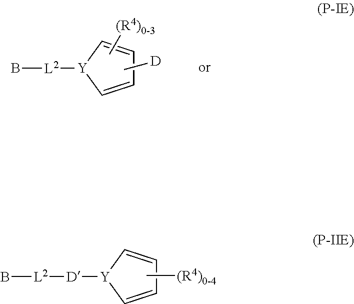

or a pharmaceutically acceptable salt thereof, wherein D, D′, Y, B, L², and R⁴ are each as defined herein including embodiments.

In embodiments, an ADC of formula (IF) or formula (IIF) can be prepared by reacting a monoclonal antibody (Ab) with a molecule of formula (P-IF) or formula (P-IIF):

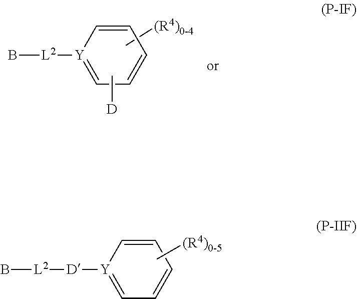

or a pharmaceutically acceptable salt thereof, wherein D, D′, Y, B, L², and R⁴ are each as defined herein including embodiments.

In embodiments, provided herein is an ADC of formula (IG) or formula (IH):

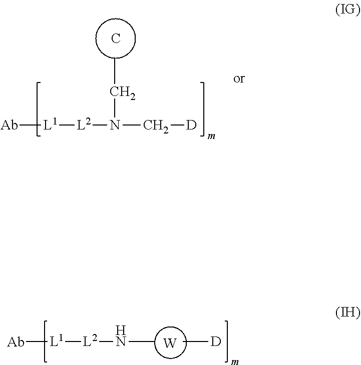

or a pharmaceutically acceptable salt thereof, wherein:
ring W is a substituted or unsubstituted cycloalkylene or a substituted or unsubstituted arylene;
ring C is a substituted or unsubstituted cycloalkyl, substituted or unsubstituted heterocycloalkyl, substituted or unsubstituted aryl, or substituted or unsubstituted heteroaryl; and wherein D, m, L¹, L², and Ab are each as defined herein including embodiments.

In embodiments, ring W is a substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene) or substituted (e.g. with a substituent group, a size-limited substituent group or a lower substituent group) or unsubstituted arylene (e.g., C₅-C₁₀ arylene, C₅-C₈ arylene, or C₅-C₆ arylene). In embodiments, ring W is substituted with one or more substituent groups. In embodiments, ring W is substituted with one or more size-limited substituent groups. In embodiments, ring W is substituted with one or more lower substituent groups.

In embodiments, ring W is a substituted (e.g., with a substituent group, a size-limited substituent group or a lower substituent group) or unsubstituted C₃-C₈ cycloalkylene. In embodiments, ring W is a substituted (e.g., with a substituent group, a size-limited substituent group or a lower substituent group) C₃-C₈ cycloalkylene. In embodiments, ring W is an unsubstituted C₃-C₈ cycloalkylene.

In embodiments, ring W is a substituted with one or more (e.g., with a substituent group, a size-limited substituent group or a lower substituent group) C₃-C₈ cycloalkylene.

In embodiments, ring W is a substituted (e.g., with a substituent group, a size-limited substituent group or a lower substituent group) or unsubstituted cyclobutylene. In embodiments, ring W is a substituted (e.g., with a substituent group, a size-limited substituent group or a lower substituent group) or unsubstituted cyclopentylene. In embodiments, ring W is a substituted (e.g., with a substituent group, a size-limited substituent group or a lower substituent group) or unsubstituted cyclohexylene.

In embodiments, ring W is a substituted (e.g., with a substituent group, a size-limited substituent group or a lower substituent group) or unsubstituted C₅-C₆ arylene. In embodiments, ring W is a substituted (e.g., with a substituent group, a size-limited substituent group or a lower substituent group) C₅-C₆ arylene. In embodiments, ring W is an unsubstituted C₅-C₆ arylene. In embodiments, ring W is a substituted with one or more (e.g., with a substituent group, a size-limited substituent group or a lower substituent group) C₅-C₆ arylene.

In embodiments, ring C is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, ring C is substituted with one or more substituent groups. In embodiments, ring C is substituted with one or more size-limited substituent groups. In embodiments, ring C is substituted with one or more lower substituent groups.

In embodiments, ring C is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, ring C is substituted with one or more substituent groups. In embodiments, ring C is substituted with one or more size-limited substituent groups. In embodiments, ring C is substituted with one or more lower substituent groups.

In embodiments, ring C is a substituted with one or more (e.g., with a substituent group, a size-limited substituent group or a lower substituent group) 5 to 9 membered heteroaryl. In embodiments, ring C is an unsubstituted 5 to 9 membered heteroaryl.

In embodiments, ring C is a substituted with one or more (e.g., with a substituent group, a size-limited substituent group or a lower substituent group) 5 to 6 membered heteroaryl. In embodiments, ring C is an unsubstituted 5 to 6 membered heteroaryl.

In embodiments, ring C is a substituted with one or more (e.g., with a substituent group, a size-limited substituent group or a lower substituent group) 3 to 8 membered heterocycloalkyl. In embodiments, ring C is a substituted with one or more (e.g., with a substituent group, a size-limited substituent group or a lower substituent group) 5 to 6 membered heterocycloalkyl.

In embodiments, ring C is substituted (e.g., with a substituent group, a size-limited substituent group or a lower substituent group) or unsubstituted furanyl, pyrrolyl, pyridyl, pyranyl, imidazolyl, thienyl, oxazolyl, or thiazolyl. In embodiments, ring C is substituted (e.g., with a substituent group, a size-limited substituent group or a lower substituent group) furanyl, pyrrolyl, pyridyl, pyranyl, imidazolyl, thienyl, oxazolyl, or thiazolyl. In embodiments, ring C is unsubstituted furanyl, pyrrolyl, pyridyl, pyranyl, imidazolyl, thienyl, oxazolyl, or thiazolyl.

In embodiments, ring C is substituted (e.g., with a substituent group, a size-limited substituent group or a lower substituent group) or unsubstituted furanyl. In embodiments, ring C is substituted (e.g., with a substituent group, a size-limited substituent group or a lower substituent group) furanyl. In embodiments, ring C is unsubstituted furanyl.

In embodiments, ring C is substituted (e.g., with a substituent group, a size-limited substituent group or a lower substituent group) or unsubstituted pyrrolyl. In embodiments, ring C is substituted (e.g., with a substituent group, a size-limited substituent group or a lower substituent group) pyrrolyl. In embodiments, ring C is unsubstituted pyrrolyl.

In embodiments, ring C is substituted (e.g., with a substituent group, a size-limited substituent group or a lower substituent group) or unsubstituted pyridyl. In embodiments, ring C is substituted (e.g., with a substituent group, a size-limited substituent group or a lower substituent group) pyridyl. In embodiments, ring C is unsubstituted pyridyl.

In embodiments, ring C is substituted (e.g., with a substituent group, a size-limited substituent group or a lower substituent group) or unsubstituted pyranyl. In embodiments, ring C is substituted (e.g., with a substituent group, a size-limited substituent group or a lower substituent group) pyranyl. In embodiments, ring C is unsubstituted pyranyl.

In embodiments, ring C is substituted (e.g., with a substituent group, a size-limited substituent group or a lower substituent group) or unsubstituted imidazolyl. In embodiments, ring C is substituted (e.g., with a substituent group, a size-limited substituent group or a lower substituent group) imidazolyl. In embodiments, ring C is unsubstituted imidazolyl.

In embodiments, ring C is substituted (e.g., with a substituent group, a size-limited substituent group or a lower substituent group) or unsubstituted thiazolyl. In embodiments, ring C is substituted (e.g., with a substituent group, a size-limited substituent group or a lower substituent group) thiazolyl. In embodiments, ring C is unsubstituted thiazolyl.

In embodiments, ring C is substituted (e.g., with a substituent group, a size-limited substituent group or a lower substituent group) or unsubstituted thienyl. In embodiments, ring C is substituted (e.g., with a substituent group, a size-limited substituent group or a lower substituent group) thienyl. In embodiments, ring C is unsubstituted thienyl.

In embodiments, ring C is substituted (e.g., with a substituent group, a size-limited substituent group or a lower substituent group) or unsubstituted oxazolyl. In embodiments, ring C is substituted (e.g., with a substituent group, a size-limited substituent group or a lower substituent group) oxazolyl. In embodiments, ring C is unsubstituted oxazolyl.

In embodiments, ring C is a substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl) or substituted (e.g. with a substituent group, a size-limited substituent group or a lower substituent group) or unsubstituted aryl (e.g., C₅-C₁₀ aryl, C₅-C₈ aryl, or C₅-C₆ aryl). In embodiments, ring C is substituted with one or more substituent groups. In embodiments, ring C is substituted with one or more size-limited substituent groups. In embodiments, ring C is substituted with one or more lower substituent groups.

In embodiments, ring C is a substituted (e.g., with a substituent group, a size-limited substituent group or a lower substituent group) or unsubstituted C₃-C₈ cycloalkyl. In embodiments, ring C is a substituted (e.g., with a substituent group, a size-limited substituent group or a lower substituent group) C₃-C₈ cycloalkyl. In embodiments, ring C is an unsubstituted C₃-C₈ cycloalkyl. In embodiments, ring C is a substituted with one or more (e.g., with a substituent group, a size-limited substituent group or a lower substituent group) C₃-C₈ cycloalkyl.

In embodiments, ring C is a substituted (e.g., with a substituent group, a size-limited substituent group or a lower substituent group) or unsubstituted cyclobutyl. In embodiments, ring C is a substituted (e.g., with a substituent group, a size-limited substituent group or a lower substituent group) or unsubstituted cyclopentyl. In embodiments, ring C is a substituted (e.g., with a substituent group, a size-limited substituent group or a lower substituent group) or unsubstituted cyclohexyl.

In embodiments, ring C is a substituted (e.g., with a substituent group, a size-limited substituent group or a lower substituent group) or unsubstituted C₅-C₆ aryl. In embodiments, ring C is a substituted (e.g., with a substituent group, a size-limited substituent group or a lower substituent group) C₅-C₆ aryl. In embodiments, ring C is an unsubstituted C₅-C₆ aryl. In embodiments, ring C is a substituted with one or more (e.g., with a substituent group, a size-limited substituent group or a lower substituent group) C₅-C₆ aryl.

In embodiments, provided herein is an ADC of formula (IJ) or formula (IK):

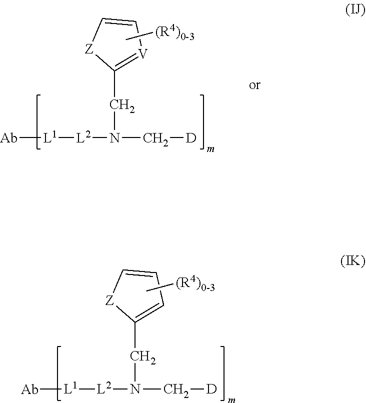

or a pharmaceutically acceptable salt thereof, wherein:
Z is S, N, or O; V is C or N; and wherein D, m, L¹, L², R⁴, and Ab are each as defined herein including embodiments.

In embodiments, Z is N. In embodiments, Z is O. In embodiments, Z is S.

In embodiments, V is C. In embodiments, V is N.

In embodiments, provided herein is an ADC of formula (IL) or formula (IM):

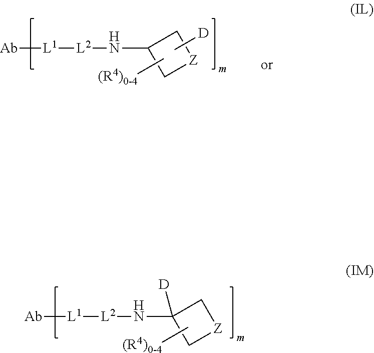

or a pharmaceutically acceptable salt thereof, wherein D, Z, m, L¹, L², R⁴, and Ab are each as defined herein including embodiments.

In embodiments, provided herein is an ADC of formula (IN) or formula (IO):

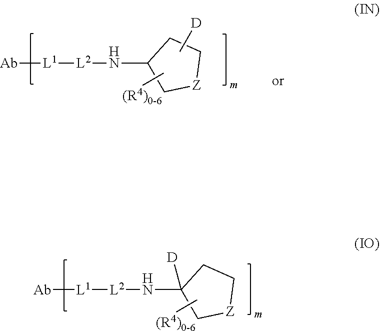

or a pharmaceutically acceptable salt thereof, wherein D, Z, m, L¹, L², R⁴, and Ab are each as defined herein including embodiments.

In embodiments, provided herein is an ADC of formula (TP) or formula (IQ):

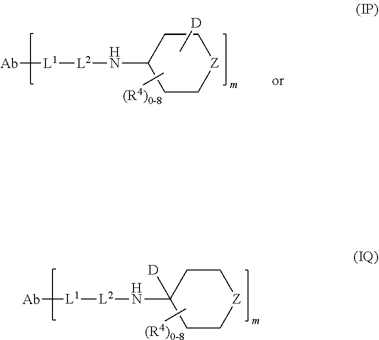

or a pharmaceutically acceptable salt thereof, wherein D, Z, m, L¹, L², R⁴, and Ab are each as defined herein including embodiments.

In embodiments, B-L²-L³-D (P-I) is a molecule of formula:

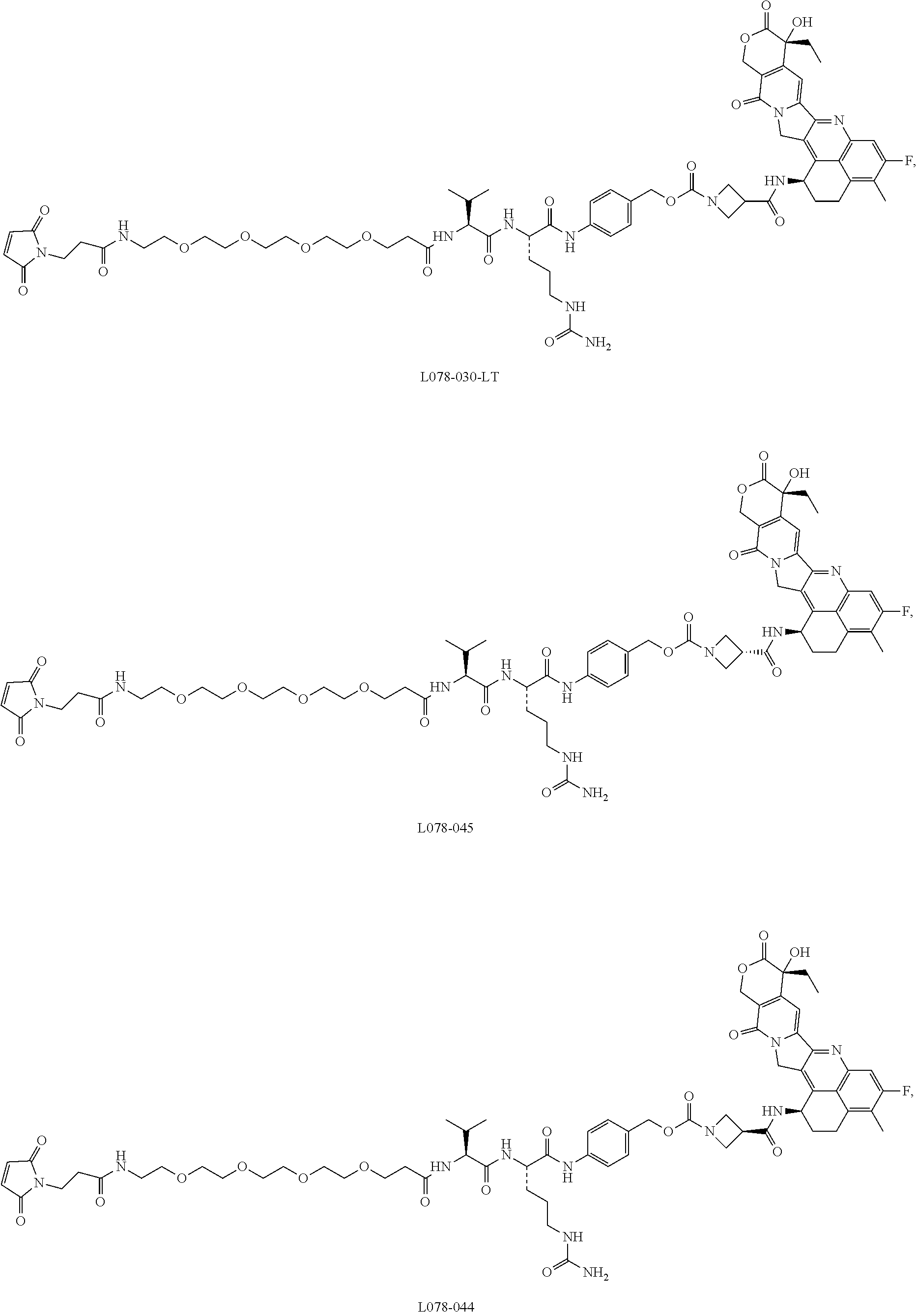

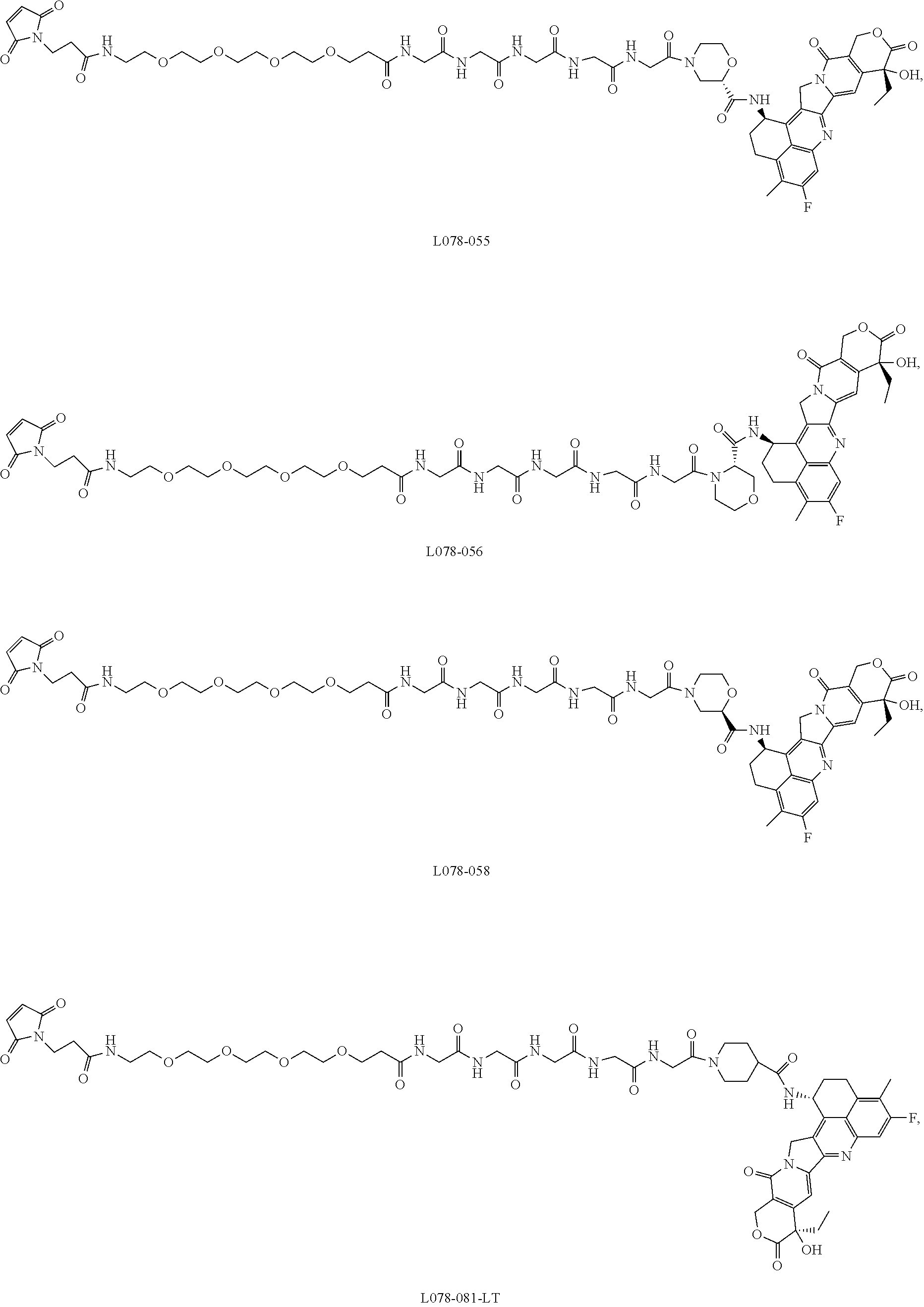

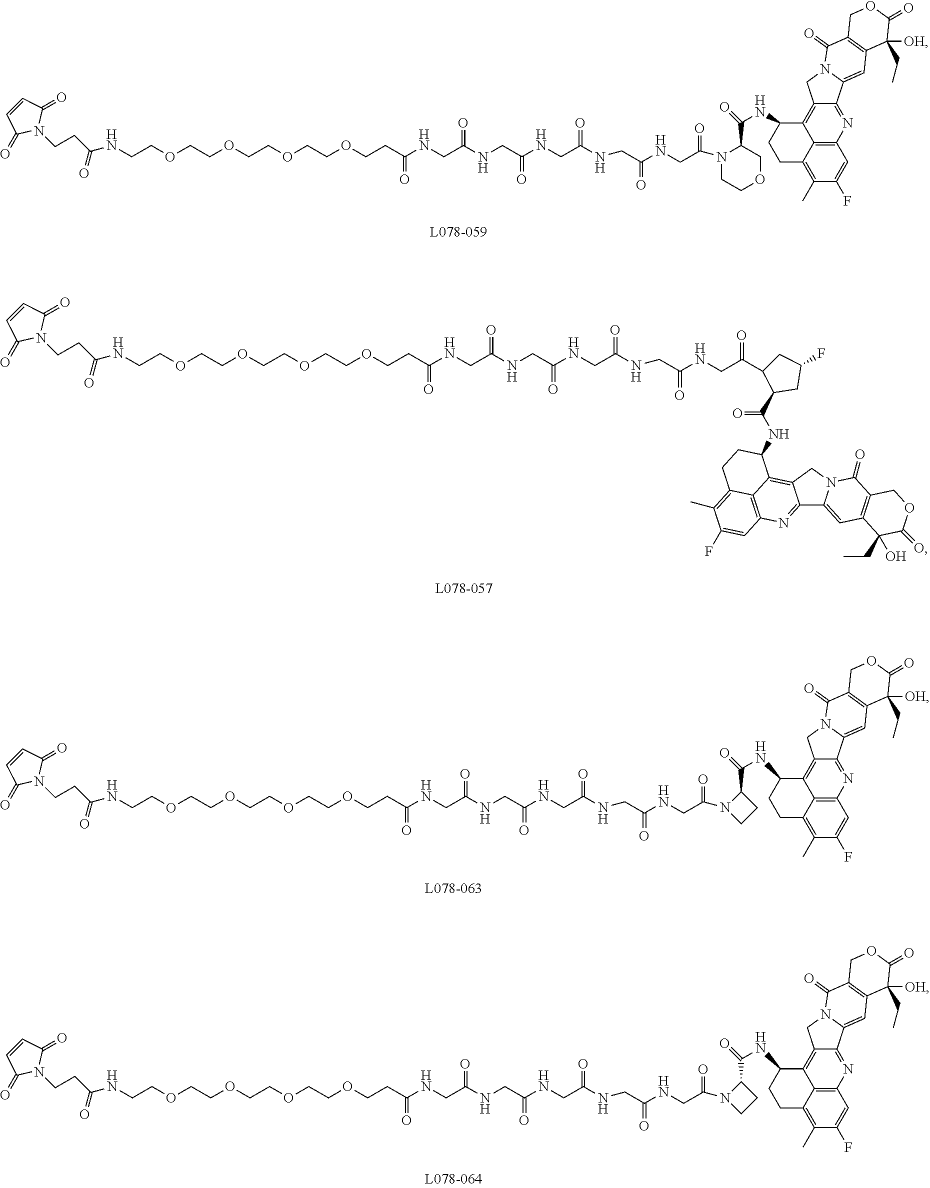

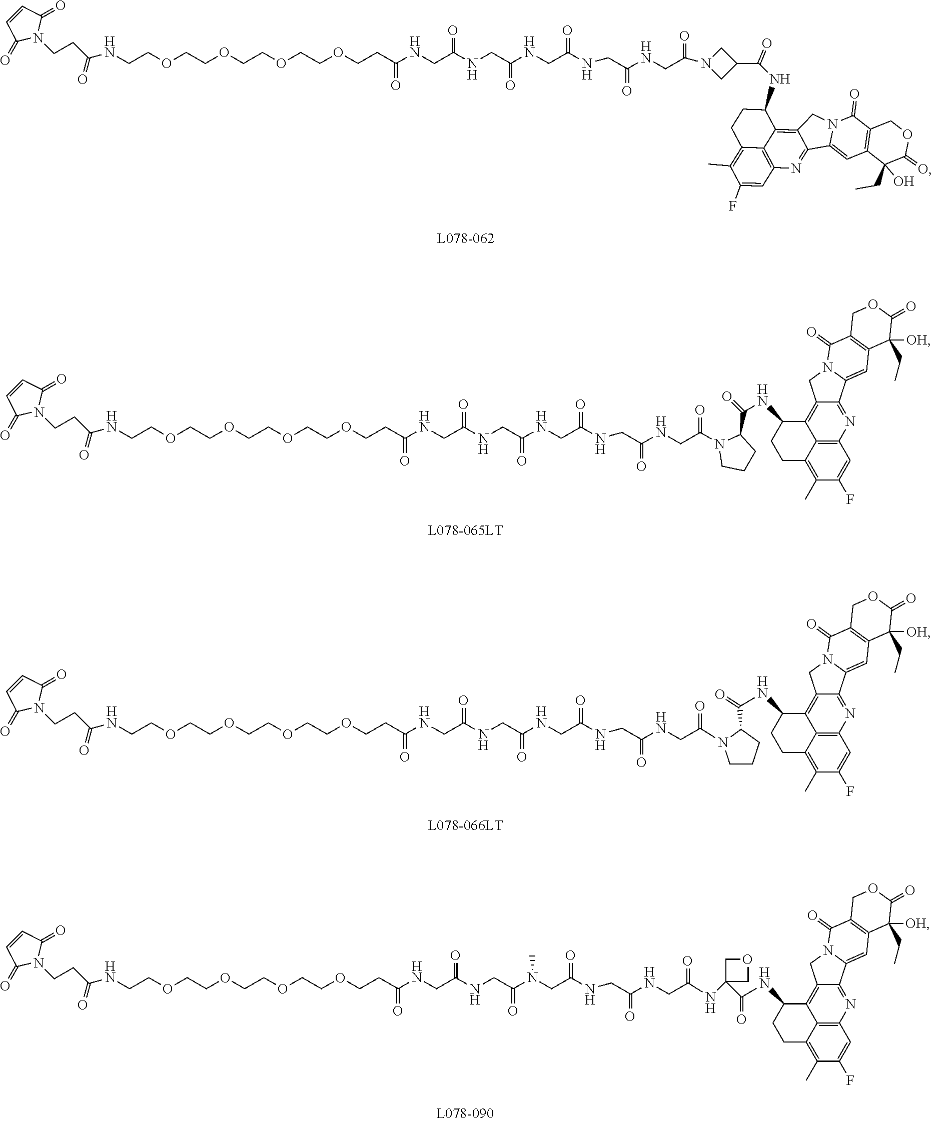

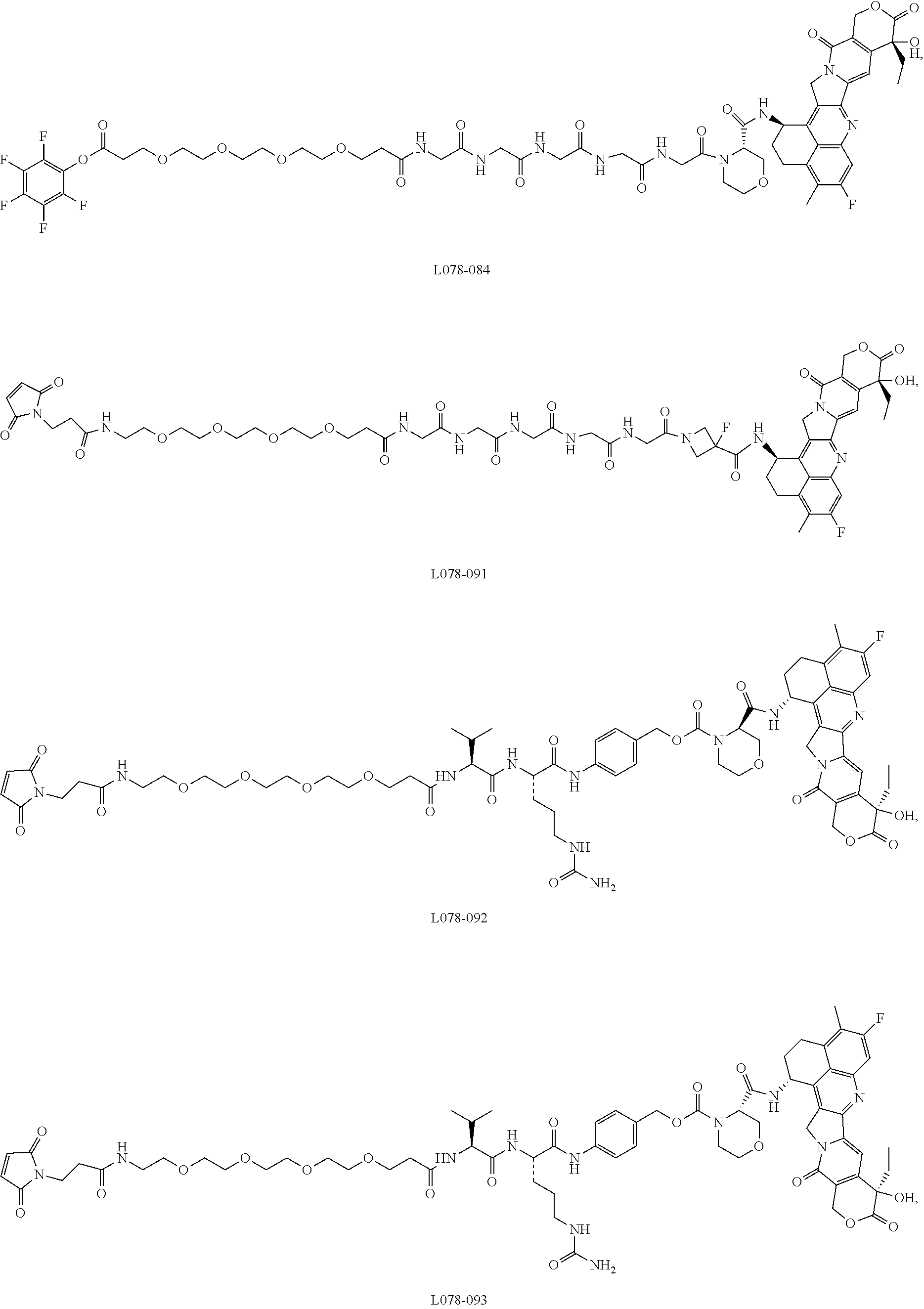

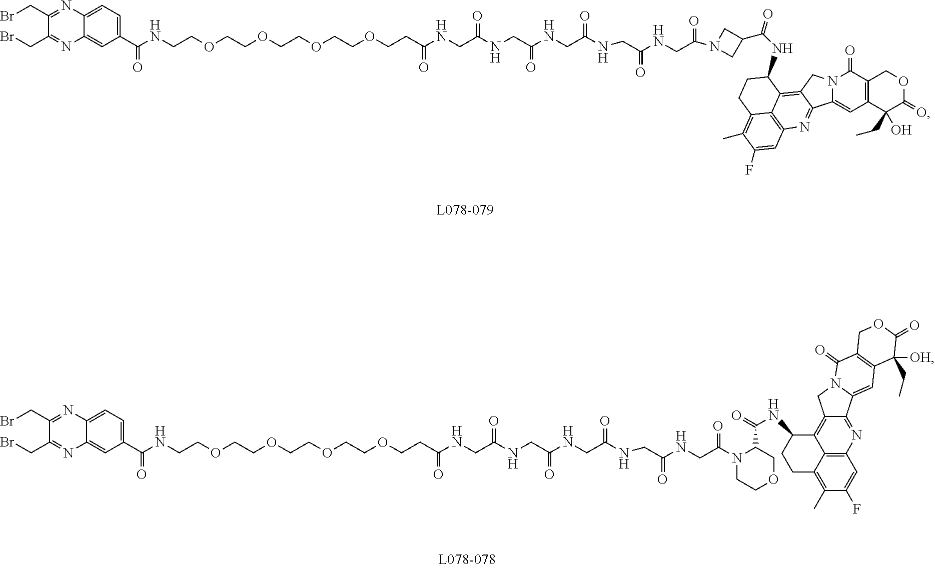

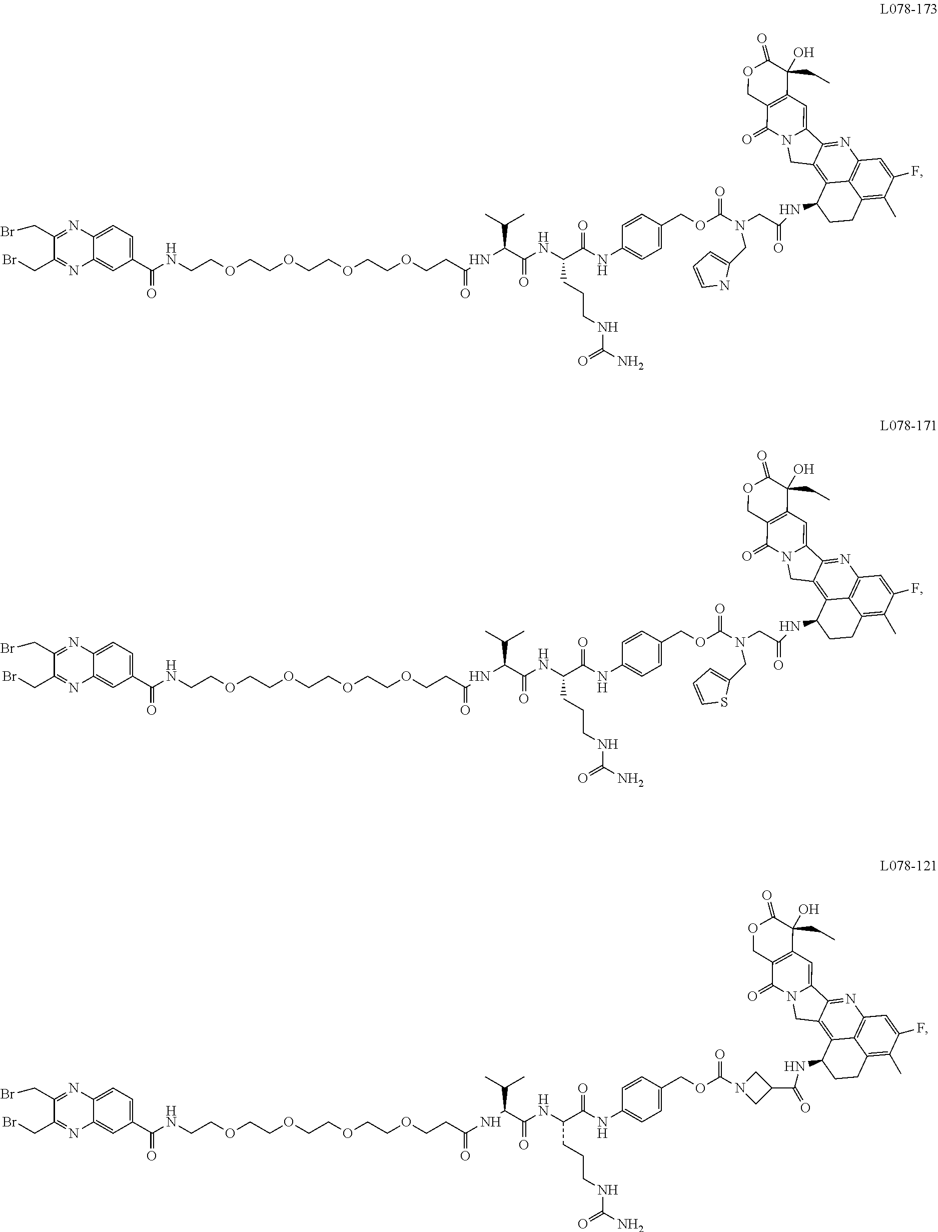

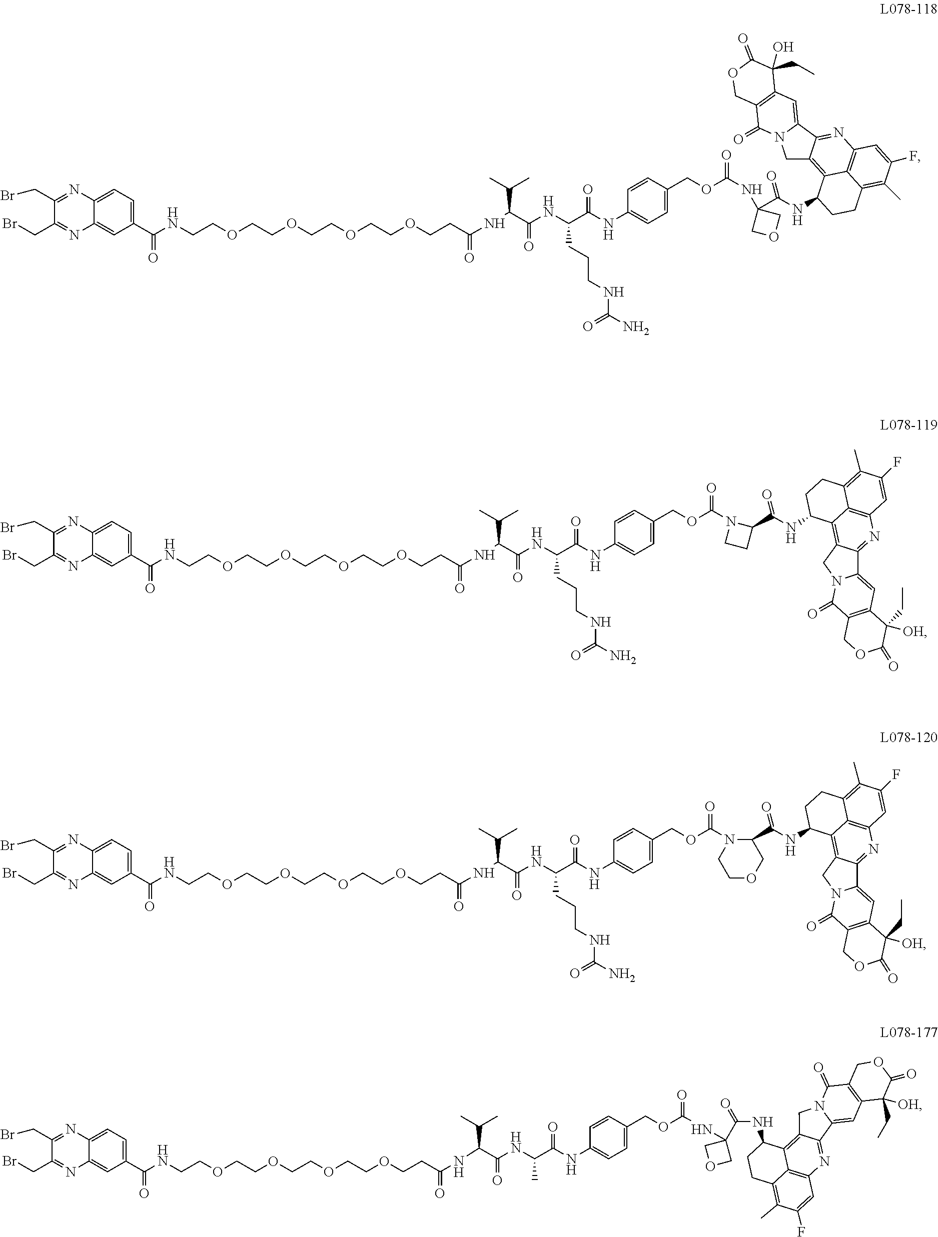

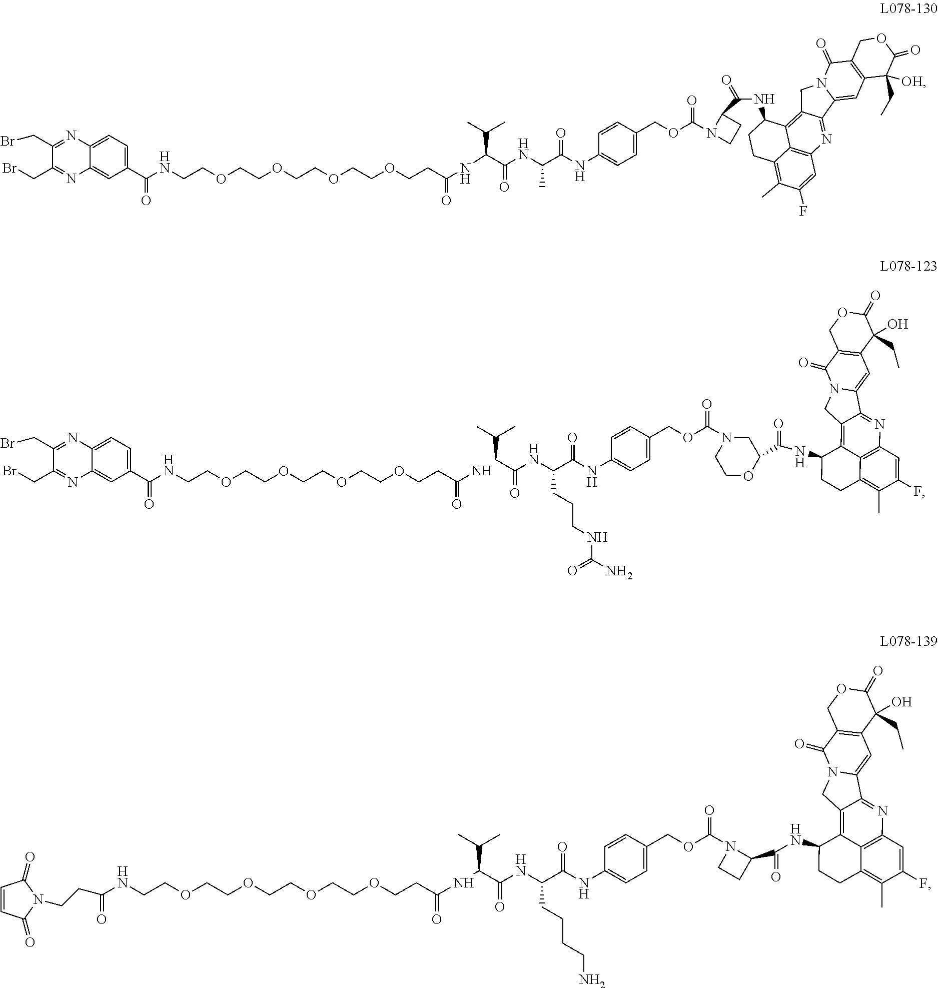

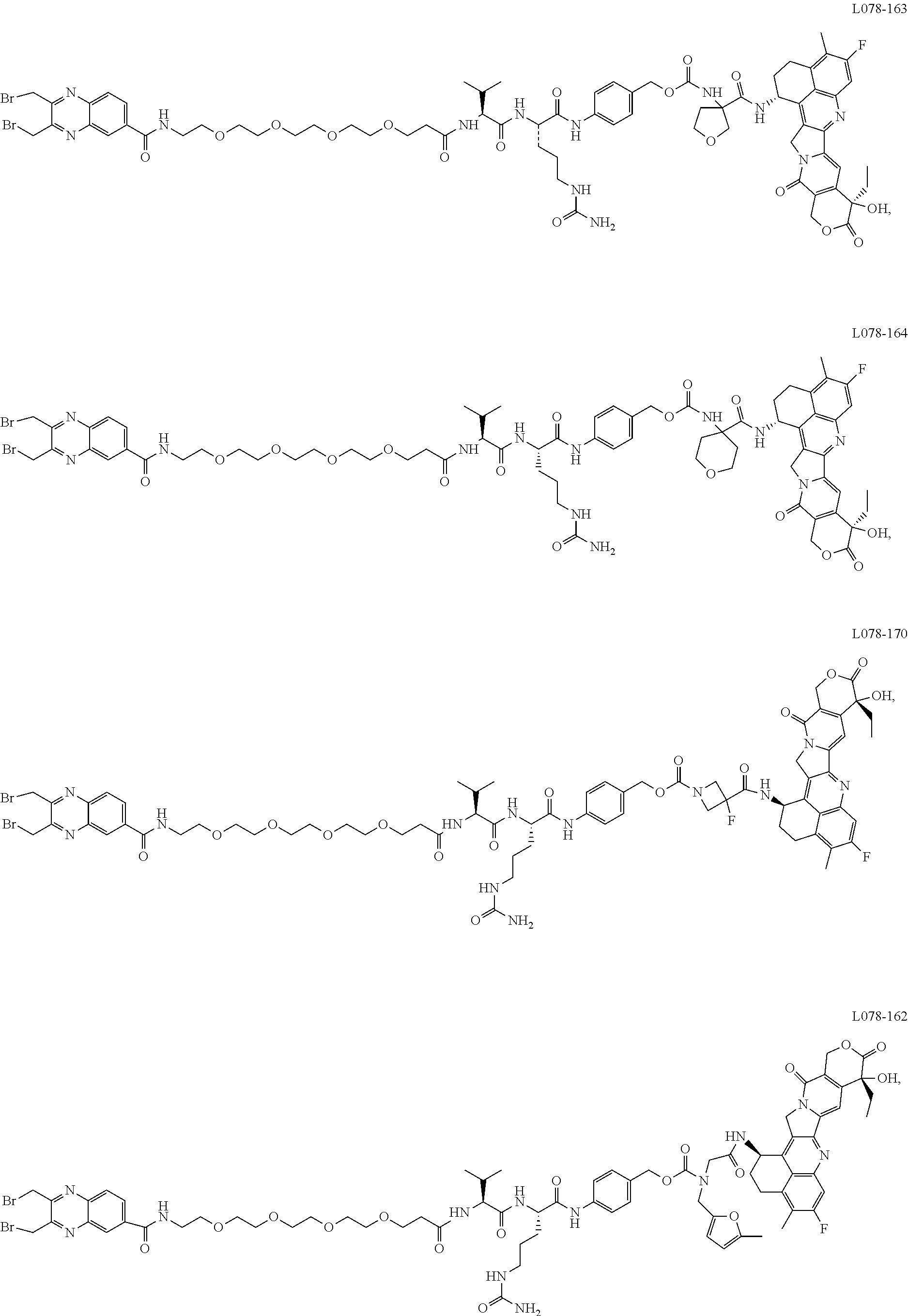

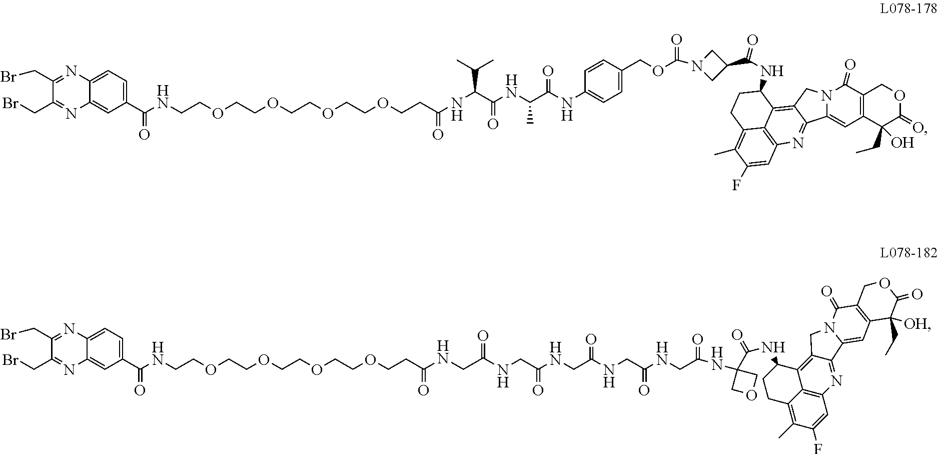

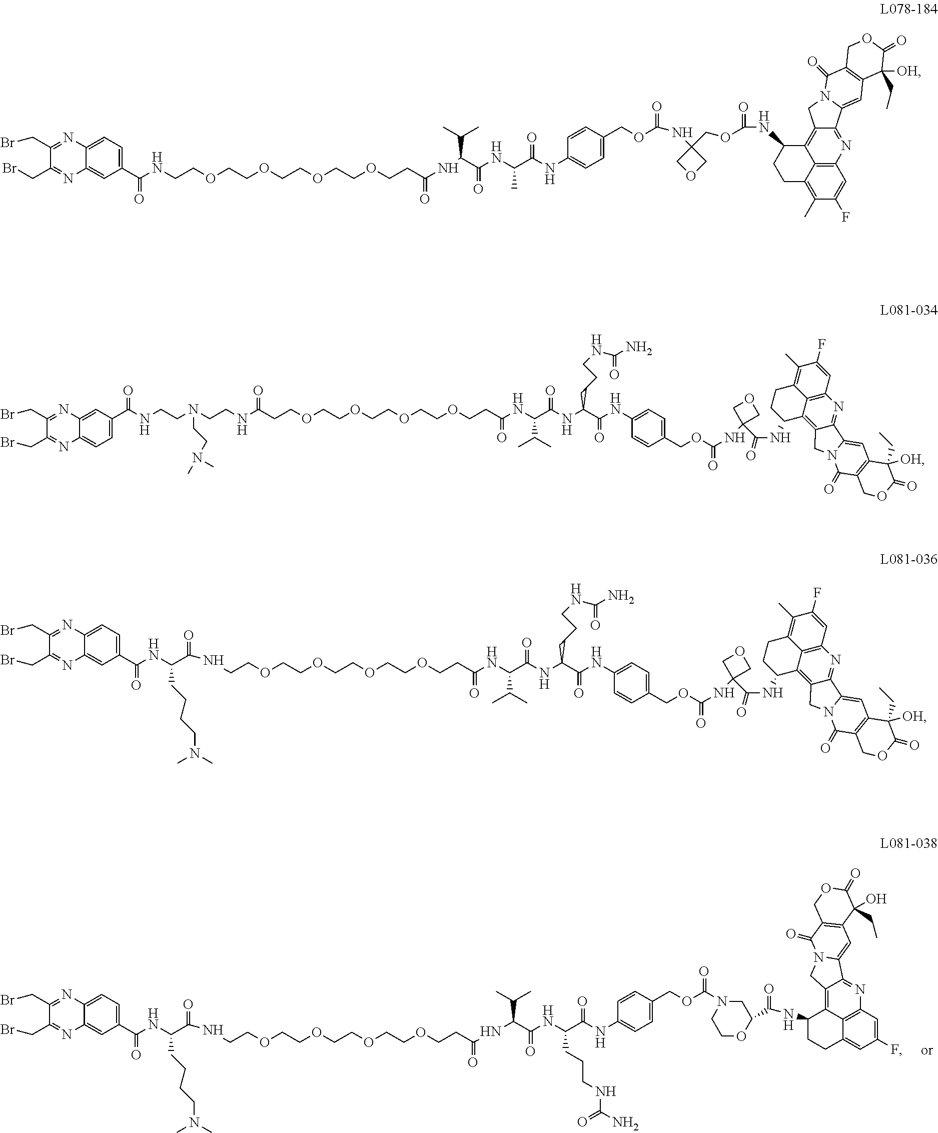

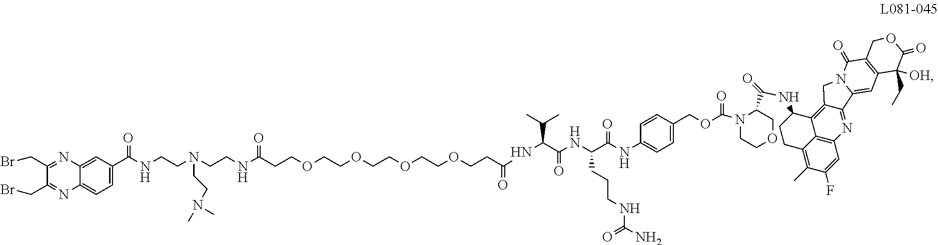 [SMILES: CC[C@]1(O)C(=O)OCc2c1cc1n(c2=O)Cc2c-1nc1cc(F)c(C)c3c1c2[C@H](NC(=O)[C@@H]1COCCN1C(=O)OCc1ccc(NC(=O)[C@H](CCCNC(N)=O)NC(=O)[C@@H](NC(=O)CCOCCOCCOCCOCCC(=O)NCCN(CCNC(=O)c2ccc4nc(CBr)c(CBr)nc4c2)CCN(C)C)C(C)C)cc1)CC3]

or a pharmaceutically acceptable salt thereof.

In embodiments, B-L²-D′-R¹ (P-II) is a molecule of formula:

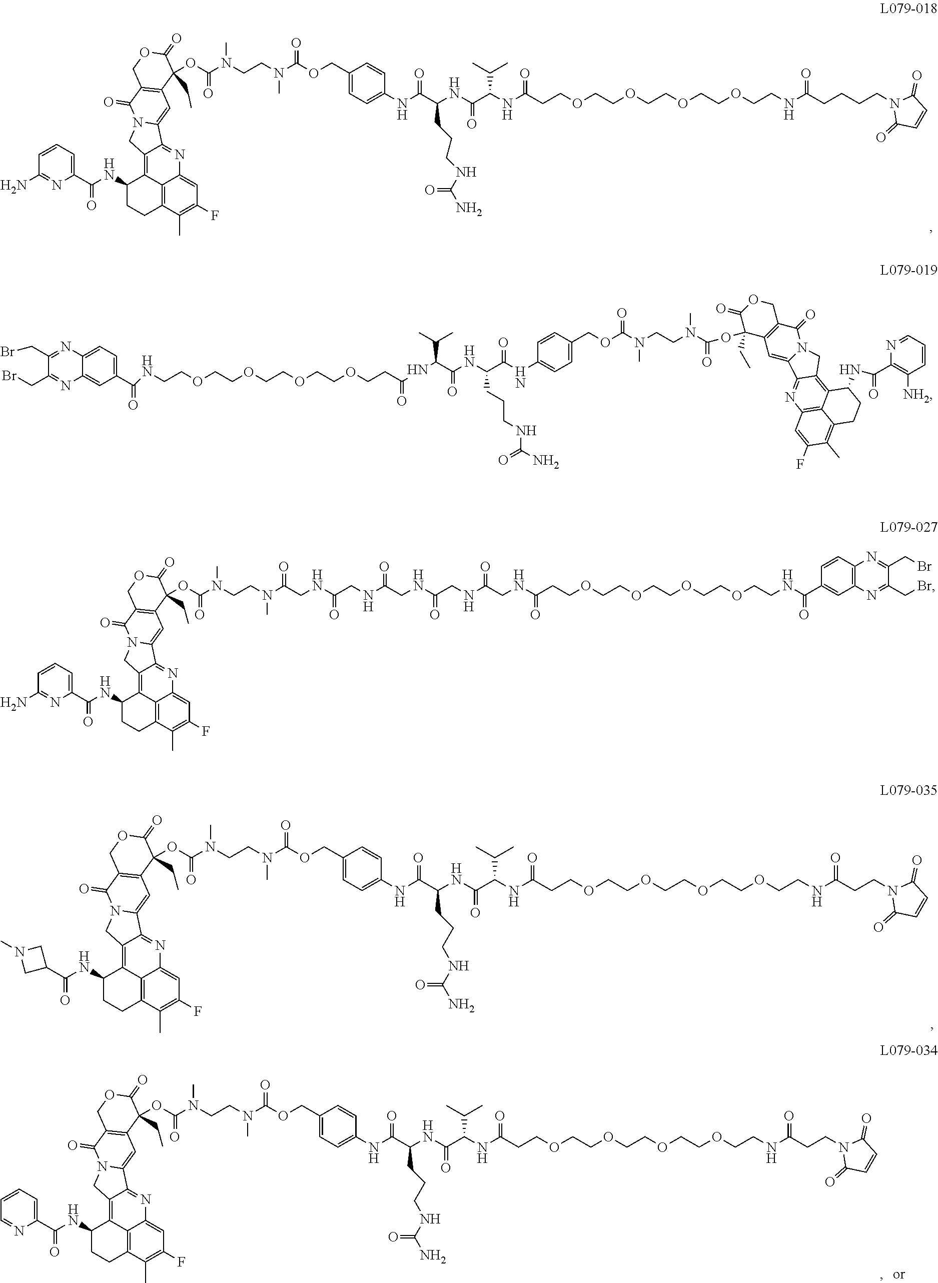

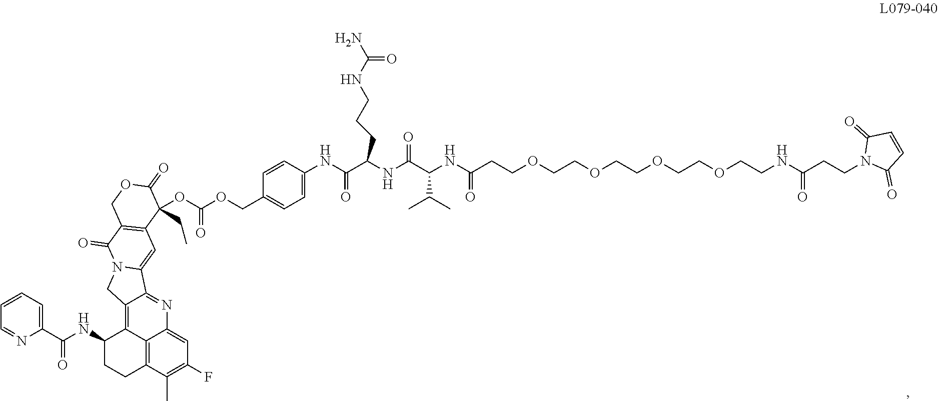 [SMILES: CC[C@]1(OC(=O)OCc2ccc(NC(=O)[C@@H](CCCNC(N)=O)NC(=O)[C@H](NC(=O)CCOCCOCCOCCOCCNC(=O)CCN3C(=O)C=CC3=O)C(C)C)cc2)C(=O)OCc2c1cc1n(c2=O)Cc2c-1nc1cc(F)c(C)c3c1c2[C@H](NC(=O)c1ccccn1)CC3]

or a pharmaceutically acceptable salt thereof.

### Pharmaceutical Compositions

In an aspect, provided herein is a pharmaceutical composition including an ADC as described herein, including embodiments, and a pharmaceutically acceptable carrier. In embodiments, the ADC as described herein is included in a therapeutically effective amount.

In embodiments, the pharmaceutical composition is formulated as a tablet, a powder, a capsule, a pill, a cachet, or a lozenge as described herein. The pharmaceutical composition may be formulated as a tablet, capsule, pill, cachet, or lozenge for oral administration. The pharmaceutical composition may be formulated for dissolution into a solution for administration by such techniques as, for example, intravenous administration. The pharmaceutical composition may be formulated for oral administration, suppository administration, topical administration, intravenous administration, intraperitoneal administration, intramuscular administration, intralesional administration, intrathecal administration, intranasal administration, subcutaneous administration, implantation, transdermal administration, or transmucosal administration as described herein.

The ADCs and pharmaceutical compositions thereof are particularly useful for parenteral administration, i.e., subcutaneously (s.c.), intrathecally, intraperitoneally, intramuscularly (i.m.) or intravenously (i.v.). In embodiment, the ADCs and pharmaceutical compositions thereof are administered intravenously or subcutaneously.

The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as about 15 or 20% by weight and will be selected primarily based on fluid volumes, viscosities, etc., according to the particular mode of administration selected.

Actual methods for preparing parenterally administrable compositions are well known or will be apparent to those skilled in the art and are described in more detail in, for example, Remington's Pharmaceutical Science, 15^{th} ed., Mack Publishing Company, Easton, Pa. For the preparation of intravenously administrable antigen binding protein formulations of the invention see Lasmar U and Parkins D “The formulation of Biopharmaceutical products”, Pharma. Sci. Tech. today, page 129-137, Vol. 3 (3 Apr. 2000); Wang, W “Instability, stabilisation and formulation of liquid protein pharmaceuticals”, Int. J. Pharm 185 (1999) 129-188; Stability of Protein Pharmaceuticals Part A and B ed Ahern T. J., Manning M. C., New York, N.Y.: Plenum Press (1992); Akers, M. J. “Excipient-Drug interactions in Parenteral Formulations”, J. Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J. Pharm. Pharmacol, 54 (2002) 1033-1039; Johnson, R, “Mannitol-sucrose mixtures-versatile formulations for protein peroxidise19g19n”, J. Pharm. Sci, 91 (2002) 914-922; and Ha, E Wang W, Wang Y. j. “Peroxide formation in polysorbate 80 and protein stability”, J. Pharm Sci, 91, 2252-2264, (2002) the entire contents of which are incorporated herein by reference and to which the reader is specifically referred.

In embodiments, the pharmaceutical composition may include optical isomers, diastereomers, enantiomers, isoforms, polymorphs, hydrates, solvates or products, or pharmaceutically acceptable salts of the compound described herein. The compound described herein (including pharmaceutically acceptable salts thereof) included in the pharmaceutical composition may be covalently attached to a carrier moiety, as described above. In embodiments, the compound described herein (including pharmaceutically acceptable salts thereof) included in the pharmaceutical composition is not covalently linked to a carrier moiety. A combination of covalently and not covalently linked compound described herein may be in a pharmaceutical composition herein.

### Methods of Use

Amplification or overexpression of the HER2 gene occurs in approximately 15-30% of breast cancers (Burstein H. J., 2005, N. Engl. J. Med. 353(16):1652-1654). With increasing understanding of HER2 biology, it has now been recognized that HER2 overexpression occurs in other forms of cancers also such as stomach, ovary, uterine serous endometrial carcinoma, colon, bladder, lung, uterine cervix, head and neck, and esophagus (Fukushige S. I. et al., 1986, Mol. Cell Biol. 6(3):955-958; Reichelt U. et al., 2007, Mod Pathol. 20(1):120-129).

HER2 is overexpressed in 15-30% of invasive breast cancers, which has both prognostic and predictive implications (Burstein H. J., 2005, N. Engl. J. Med. 353(16):1652-1654). Amplification of HER2 gene was found to be a significant predictor of both overall survival (P<0.001) and time to relapse (P<0.0001). In a study by Press et al. (Press M. F. et al., 1993, Cancer Res. 53(20):4960-4970), the expression of HER2 was studied in 704 node-negative breast cancers and it was found that women with breast cancer having high overexpression had a risk of recurrence 9.5 times greater than those whose breast cancers had normal expression (P=0.0001). Seshadri et al. (Seshadri R. et al., 1993, J. Clin. Oncol. 11(10):1936-1942) in their study of 1056 patients with Stages I-III breast cancer found that HER2 amplification 3-fold or greater was associated with significantly shorter disease-free survival (P=0.0027). HER2 amplification also correlated significantly with pathologic stage of disease, number of axillary nodes with tumor, histologic type, and absence of estrogen receptor (ER) and progesterone receptor (PgR). Evidence suggests that HER2 amplification is an early event in human breast tumorigenesis.

In an aspect, provided herein is a method of treating a disease in a subject in need thereof, said method including administering an effective amount of an antibody drug conjugate (ADC) comprising an IgG antibody, a conjugation linker moiety (L¹) that binds to the thiol of cysteine residues or to the amine of lysine residues of the IgG antibody, and to a drug moiety covalently bound to L³-L²-L¹, or a drug moiety separately bound to both L²-L¹ and R¹. In embodiments, the IgG antibody binds to HER2.

In one aspect, an ADC provided herein is used in a method of inhibiting proliferation of a HER2-expressing cell, the method comprising exposing the cell to the ADC under conditions permissive for binding of the anti-HER2 antibody of the ADC on the surface of the cell, thereby inhibiting the proliferation of the cell. In embodiments, the method is an in vitro or an in vivo method. In embodiments, the cell is a B cell.

Inhibition of cell proliferation in vitro may be assayed using the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al. (1993) J. Immunol. Meth. 160:81-88, U.S. Pat. No. 6,602,677. The assay may be conducted in 96- or 384-well format, making it amenable to automated high-throughput screening (HTS). See Cree et al. (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

In another aspect, an ADC for use as a medicament is provided. In further aspects, an ADC for use in a method of treatment is provided. In another aspect, provided herein is a method of treating a disease in a subject in need thereof, said method including administering an effective amount of a pharmaceutical composition of the ADC as described herein.

In embodiments, the disease is cancer. In embodiments, the cancer is associated with overexpression of HER2, ROR1, CD25, TROP2, B7-H3, c-Met, FOLR1, or CHOP2. In embodiments, the cancer is associated with overexpression of HER2. In embodiments, the cancer is associated with overexpression of ROR1. In embodiments, the cancer is associated with overexpression of CD25. In embodiments, the cancer is associated with overexpression of TROP2. In embodiments, the cancer is associated with overexpression of B7-H3. In embodiments, the cancer is associated with overexpression of c-Met. In embodiments, the cancer is associated with overexpression of FOLR1. In embodiments, the cancer is associated with overexpression of CHOP2. In embodiments, provided herein is an ADC for use in a method of treating an individual having a HER2-expressing cancer, the method comprising administering to the individual an effective amount of the ADC. In one such embodiment, the method further comprises administering to the individual an effective amount of at least one additional therapeutic agent.

In a further aspect, the present disclosure provides for the use of an ADC in the manufacture or preparation of a medicament. In embodiment, the medicament is for treatment of HER2-expressing cancer. In a further embodiment, the medicament is for use in a method of treating HER2-expressing cancer, the method comprising administering to an individual having HER2-expressing cancer an effective amount of the medicament. In one such embodiment, the method further comprises administering to the individual an effective amount of at least one additional therapeutic agent.

In embodiments, the methods provided herein are for treating cancer in a mammal. In embodiments, the methods provided herein are for treating cancer in a human.

In embodiments, the cancer is a solid tumor. In embodiments, HER2 expressing solid tumors include, but are not limited to, breast cancer (e.g., estrogen and progesterone receptor negative breast cancer, triple negative breast cancer (TNBC)), ovarian cancer, lung cancer (e.g., non-small cell lung cancer (NSCLC) (including adenocarcinomas, squamous cell carcinomas and large cell carcinomas) and small cell lung cancer), gastric cancer, esophageal cancer, colorectal cancer, urothelial cancer (e.g., micropapillary urothelial cancer and typical urothelial cancer), pancreatic cancer, salivary gland cancer (e.g., mucoepidermoid carcinomas, adenoid cystic carcinomas and terminal duct adenocarcinoma) and brain cancer or metastases of the aforementioned cancers (i.e., lung metastasis from HER2+ breast cancer) (Martin et al., 2014, Future Oncol. 10(8):1469-86).

In other embodiments, HER2 expressing solid tumors include bladder cancer, gastrointestinal stromal tumor, uterine cervix cancer, peritoneal cancer, liver cancer, hepatocellular cancer, colon cancer, rectal cancer, endometrial cancer, kidney cancer, vulval cancer, thyroid cancer, penis cancer, anal cancer, astrocytoma, leukemia, lymphoma, head and neck cancer, testicular cancer, cervical cancer, sarcoma, hemangioma, eye cancer, laryngeal cancer, mouth cancer, mesothelioma, skin cancer, myeloma, oral cancer, throat cancer, prostate cancer, or ductal cancer.

In some embodiments, the HER2-expressing cancer comprises a solid tumor. In some embodiments, the HER2-expressing cancer is metastatic. In some embodiments, the HER2-expressing cancer a relapsed cancer.

In embodiments, the cancer is selected from the group consisting of breast cancer, lung cancer, ovarian cancer and gastric cancer. In embodiments, breast cancer is a metastatic breast cancer or triple negative breast cancer. In embodiments, lung cancer is non-small cell lung cancer (NSCLC).

In embodiments, the cancer is breast cancer. In embodiments, the cancer is metastatic breast cancer. In embodiments, the cancer is non-small cell lung cancer (NSCLC). In embodiments, the cancer is ovarian cancer.

In embodiments, the ADCs disclosed herein can be used to treat HER2-expressing cancers that have not been previously treated with a therapeutic agent (i.e., as a first line treatment).

In embodiments, ADCs disclosed herein can be used to treat HER2-expressing cancers that are resistant to, refractory to and/or relapsed from treatment with another therapeutic agent (i.e., as a second line treatment). In embodiments, the prior treatment was trastuzumab (trastuzumab or Herceptin®) either alone or in combination with an additional therapeutic agent (i.e., a taxane such as paclitaxel, docetaxel, cabazitaxel, etc.).

In embodiments, ADCs disclosed herein can be used to treat HER2-expressing cancers that are resistant to, refractory to and/or relapsed from treatment with more than one other therapeutic agent (i.e., as a third line treatment or a fourth line treatment, etc.).

The ADCs described herein can be used either alone or in combination with other agents in a therapy. For instance, an ADC as described herein may be co-administered with at least one additional therapeutic agent. In embodiments, other therapeutic regimens may be combined with the administration of the ADC including, without limitation, radiation therapy and/or bone marrow and peripheral blood transplants, and/or a cytotoxic agent. In embodiments, a cytotoxic agent is an agent or a combination of agents such as, for example, cyclophosphamide, docetaxel, paclitaxel, hydroxydaunorubicin, adriamycin, doxorubincin, vincristine (Oncovin™) prednisolone, CHOP (combination of cyclophosphamide, doxorubicin, vincristine, and prednisolone), or trastuzumab.

Such combination therapies noted above encompass combined administration (where two or more therapeutic agents are included in the same or separate formulations), and separate administration, in which case, administration of the ADC can occur prior to, simultaneously, and/or following, administration of the additional therapeutic agent and/or adjuvant. The ADCs described herein can also be used in combination with radiation therapy.

### Articles of Manufacture

In a further aspect, provided herein is an article of manufacture containing materials useful for the treatment, prevention and/or diagnosis of the disorders described above is provided. The article of manufacture (a kit) comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is an ADC as described herein. The label or package insert indicates that the composition is used for treating the condition of choice. Moreover, the article of manufacture (a kit) may comprise (a) a first container with a composition contained therein, wherein the composition comprises an ADC as described herein; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

LIST OF SEQUENCES:

Human HER2 sequence SEQ ID NO: 16
(UniProt P04626-1)
MELAALCRWGLLLALLPPGAASTQVCTGTDMKLRLPASPETHLDMLRHL
YQGCQVVQGNLELTYLPTNASLSFLQDIQEVQGYVLIAHNQVRQVPLQR
LRIVRGTQLFEDNYALAVLDNGDPLNNTTPVTGASPGGLRELQLRSLTE
ILKGGVLIQRNPQLCYQDTILWKDIFHKNNQLALTLIDTNRSRACHPCS
PMCKGSRCWGESSEDCQSLTRTVCAGGCARCKGPLPTDCCHEQCAAGCT
GPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCY
GLGMEHLREVRAVTSANIQEFAGCKKIFGSLAFLPESFDGDPASNTAPL
QPEQLQVFETLEEITGYLYISAWPDSLPDLSVFQNLQVIRGRILHNGAY
SLTLQGLGISWLGLRSLRELGSGLALIHHNTHLCFVHTVPWDQLFRNPH
QALLHTANRPEDECVGEGLACHQLCARGHCWGPGPTQCVCSQFLRGQEC
VEECRVLQGLPREYVNARHCLPCHPECQPQNGSVTCFGPEADQCVACAH
YKDPPFCVARCPSGVKPDLSYMPIWKFPDEEGACQPCPINCTHSCVDLD
DKGCPAEQRASPLTSIISAVVGILLVVVLGVVFGILIKRRQQKIRKYTM
RRLLQETELVEPLTPSGAMPNQAQMRILKETELRKVKVLGSGAFGTVYK
GIWIPDGENVKIPVAIKVLRENTSPKANKEILDEAYVMAGVGSPYVSRL
LGICLTSTVQLVTQLMPYGCLLDHVRENRGRLGSQDLLNWCMQIAKGMS
YLEDVRLVHRDLAARNVLVKSPNHVKITDFGLARLLDIDETEYHADGGK
VPIKWMALESILRRRFTHQSDVWSYGVTVWELMTFGAKPYDGIPAREIP
DLLEKGERLPQPPICTIDVYMIMVKCWMIDSECRPRFRELVSEFSRMAR
DPQRFVVIQNEDLGPASPLDSTFYRSLLEDDDMGDLVDAEEYLVPQQGF
FCPDPAPGAGGMVHHRHRSSSTRSGGGDLTLGLEPSEEEAPRSPLAPSE
GAGSDVFDGDLGMGAAKGLQSLPTHDPSPLQRYSEDPTVPLPSETDGYV
APLTCSPQPEYVNQPDVRPQPPSPREGPLPAARPAGATLERPKTLSPGK
NGVVKDVFAFGGAVENPEYLTPQGGAAPQPHPPPAFSPAFDNLYYWDQD
PPERGAPPSTFKGTPTAENPEYLGLDVPV.

TABLE 2
Table of Sequences:

| Light chain variable: | Heavy chain variable: |
|---|---|
| HER2 SEQ ID NO: 7 | HER2 SEQ ID NO: 8 |
| DIQMTQSPSSLSASVGDRVTITCR | EVQLVESGGGLVQPGGSLRLSCAA |
| ASQDVNTAVAWYQQKPGKAPKLLI | SGFNIKDTYIHWVRQAPGKGLEWV |
| YSASFLYSGVPSRFSGSRSGTDFT | ARIYPTNGYTRYADSVKGRFTISA |
| LTISSLQPEDFATYYCQQHYTTPP | DTSKNTAYLQMNSLRAEDTAVYYC |
| TFGQGTKVEIK | SRWGGDGFYAMDYWGQGTLVTVSS |

TABLE 3
CDRs 1, 2 and 3:

HER2:
(VL-CDR1) SEQ ID NO: 1 RASQDVNTAVA
(VL-CDR2) SEQ ID NO: 2 SASFLYS
(VL-CDR3) SEQ ID NO: 3 QQHYTTPPT
(VH-CDR1) SEQ ID NO: 4 DTYIH
(VH-CDR2) SEQ ID NO: 5 RIYPTNGYTRYADSVKG
(VH-CDR3) SEQ ID NO: 6 WGGDGFYAMDY

### EXAMPLES

The following examples are meant to be illustrative and can be used to further understand embodiments of the present disclosure and should not be construed as limiting the scope of the present teachings in any way.

The chemical reactions described in the Examples can be readily adapted to prepare a number of other compounds of the present disclosure, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by utilizing other suitable reagents known in the art other than those described, or by making routing modifications of reaction conditions, reagents, and starting materials. Alternatively, other reactions disclosed herein or known in the art will be recognized as having applicability for preparing other compounds of the present disclosure. Synthesis of compound 40 and related compounds was disclosed in U.S. Pat. Nos. 10,590,165 and 9,981,046, which are incorporated herein in their entireties. All compounds that were purified by HPLC (described below) are TFA salts.

### SYNTHETIC EXAMPLES

### Example S1: Synthesis of Compound L078-030-LT

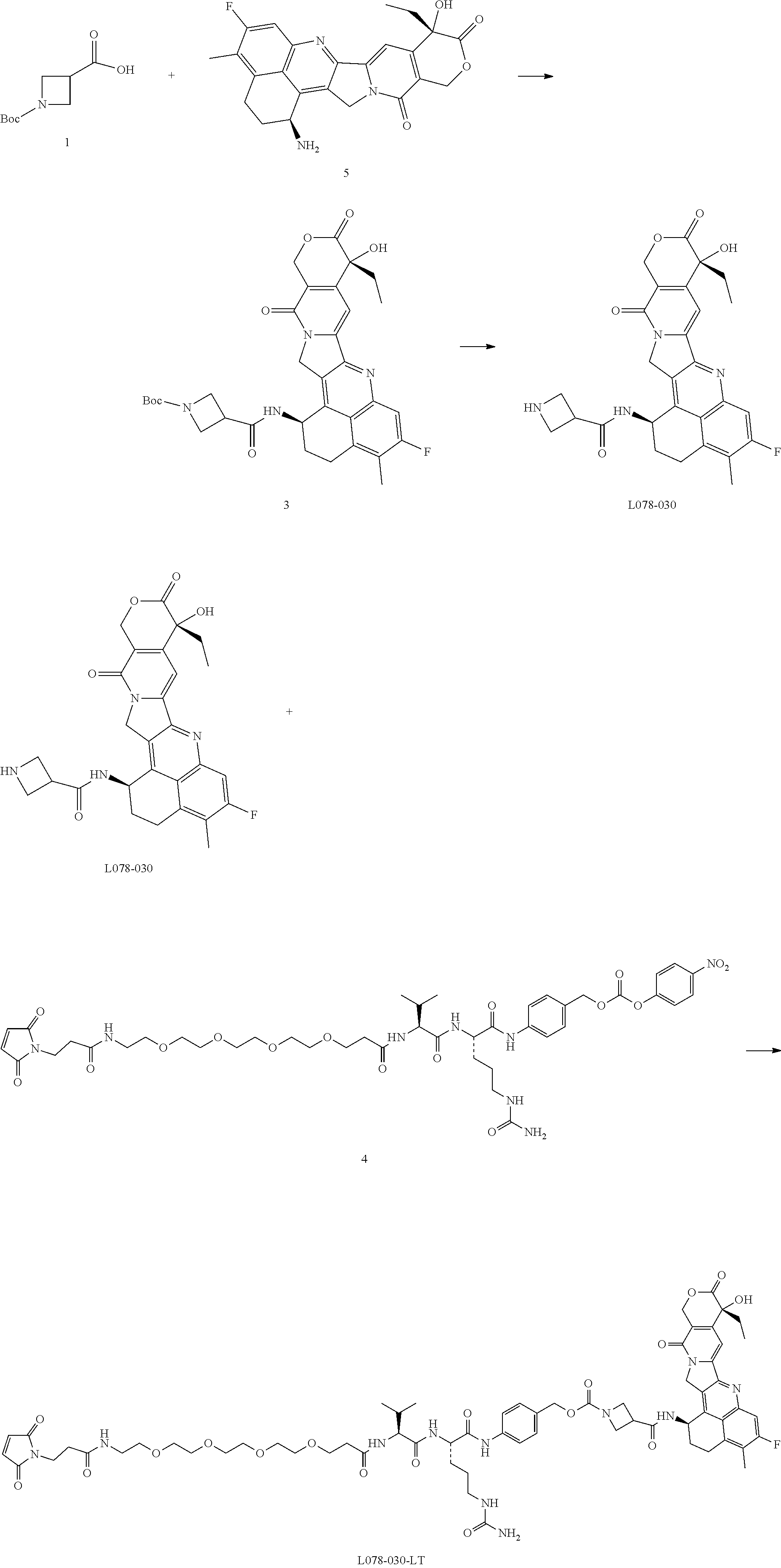

To a solution of compounds 1 (18 mg, 94 μmol), 5 (mesylate salt, 50 mg, 94 μmol) and HATU (35 mg, 94 μmol) in 2 mL of DMF was added DIEA (25 mg, 188 μmol). After the solution was stirred for 5 minutes the reaction mixture was purified by HPLC. The resulting product 3 was treated with a solution of 50% TFA in DCM for 30 minutes and evaporated under reduced pressure to dry. The residue was purified by HPLC to give compound L078-030 (TFA salt, 32 mg) as a white powder. MS m/z 519.4 (M+H).

A solution of compound L078-030 (TFA salt, 12 mg, 19 μmol), compound 4 (CAS 2003260-12-4; 22 mg, 23 mol), HOBt (5 mg) and DIEA (6 mg) in 2 mL of DMF was stirred for 1 hour. The solution was purified by HPLC to give compound L078-030-LT (8 mg) as a pale-yellow powder. MS m/z 1323.0 (M+H).

### Example S2: Synthesis of Compound L078-062

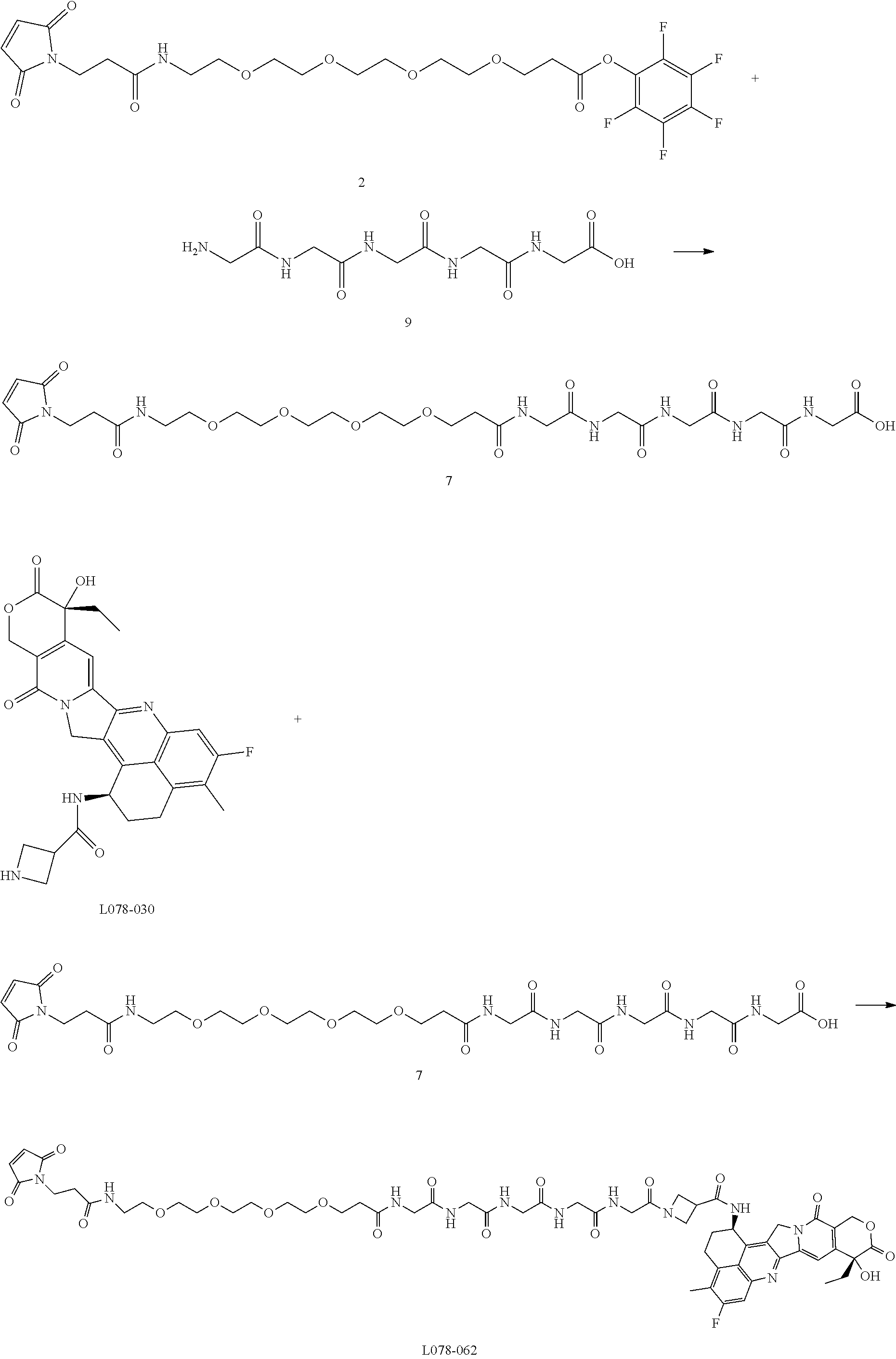 [SMILES: O=C(CCN1C(=O)C=CC1=O)NCCOCCOCCOCCOCCC(=O)Oc1c(F)c(F)c(F)c(F)c1F ; NCC(=O)NCC(=O)NCC(=O)NCC(=O)NCC(=O)O ; O=C(O)CNC(=O)CNC(=O)CNC(=O)CNC(=O)CNC(=O)CCOCCOCCOCCOCCNC(=O)CCN1C(=O)C=CC1=O ; CC[C@]1(O)C(=O)OCc2c1cc1n(c2=O)Cc2c-1nc1cc(F)c(C)c3c1c2[C@H](NC(=O)C1CNC1)CC3 ; O=C(O)CNC(=O)CNC(=O)CNC(=O)CNC(=O)CNC(=O)CCOCCOCCOCCOCCNC(=O)CCN1C(=O)C=CC1=O ; CC[C@]1(O)C(=O)OCc2c1cc1n(c2=O)Cc2c-1nc1cc(F)c(C)c3c1c2[C@H](NC(=O)C1CN(C(=O)CNC(=O)CNC(=O)CNC(=O)CNC(=O)CNC(=O)CCOCCOCCOCCOCCNC(=O)CCN2C(=O)C=CC2=O)C1)CC3]

To a solution of compound 2 (CAS #1415800-42-8; 500 mg, 0.858 mmol), compound 9 (CAS #7093-67-6; 260 mg, 0.858 mmol) in 4 mL of DMF was added 10% NaHCO₃ (and pH adjusted to 8.5). The solution was stirred for 30 minutes purified by HPLC and dried to give compound 7 (463 mg) as a white solid. MS m/z 702.3 (M+H).

To a solution of compounds 7 (10 mg, 14 μmol), L078-030 (TFA salt, 9 mg, 14 μmol) and HATU (5 mg, 14 μmol) in 2 mL of DMF was added DIEA (5 mg, 35 μmol). The solution was stirred for 5 minutes and purified by HPLC to give compound L078-062 (9 mg) as a pale-yellow powder. MS m/z 1202.6 (M+H).

### Example S3: Synthesis of Compound L078-079

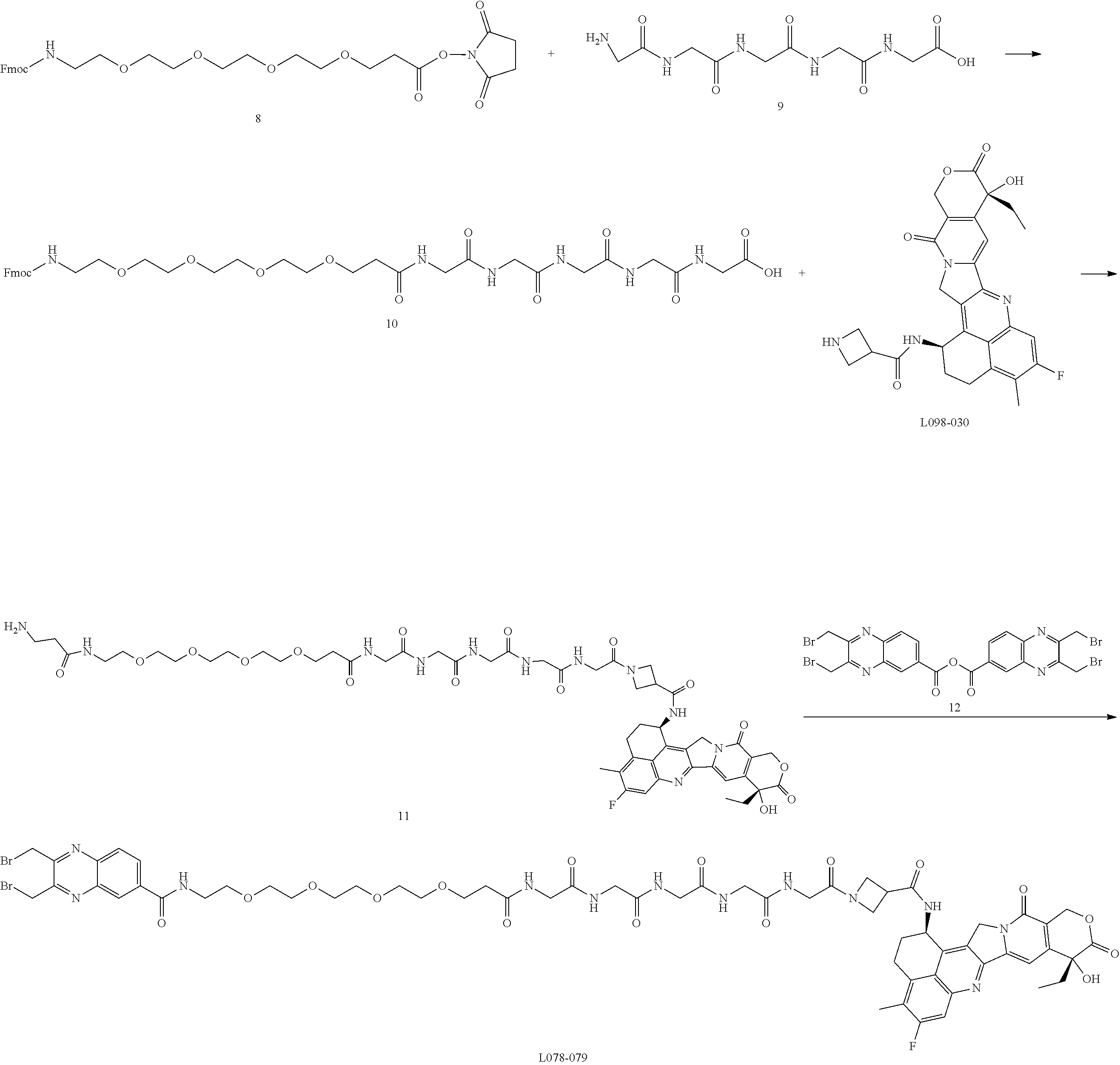

Compound 8 (2.0 g, 3.42 mmol) was dissolved in 50 mL solution of 50% CH₃CN/H₂O. To the solution was added a water solution (30 mL) of 9 (1.04 g, 3.42 mmol). To the mixture was added a saturated solution of NaHCO₃ (2 mL). The solution was stirred for 20 minutes. The white precipitate was collected by filtration, washed with water, and dried to give 10 (1.52 g). MS m/z 773.4 (M+H).

A suspension of 10 (30 mg, 39 μmol) in DMSO (6 mL) was heated to 60° C. The clear solution was cooled to room temperature. To the solution was added L078-030 (TFA salt, 20 mg, 39 μmol), HATU (15 mg, 39 μmol) and DIEA (1³ mg, 98 μmol). The solution was stirred for 5 minutes followed by addition of 1 mL diethylamine and concentrated. The residue was purified by HPLC to give 11 as a TFA salt. MS m/z 1051.9 (M+H).

A solution of compound 11 (52 mg, 39 μmol), 12 (27 mg, 39 μmol) and DIEA (13 mg, 98 μmol) was stirred for 5 minutes and purified by HPLC to give compound L078-079 (7.1 mg). MS m/z 1394.5 (M+H).

### Example S4: Synthesis of Compound L078-056

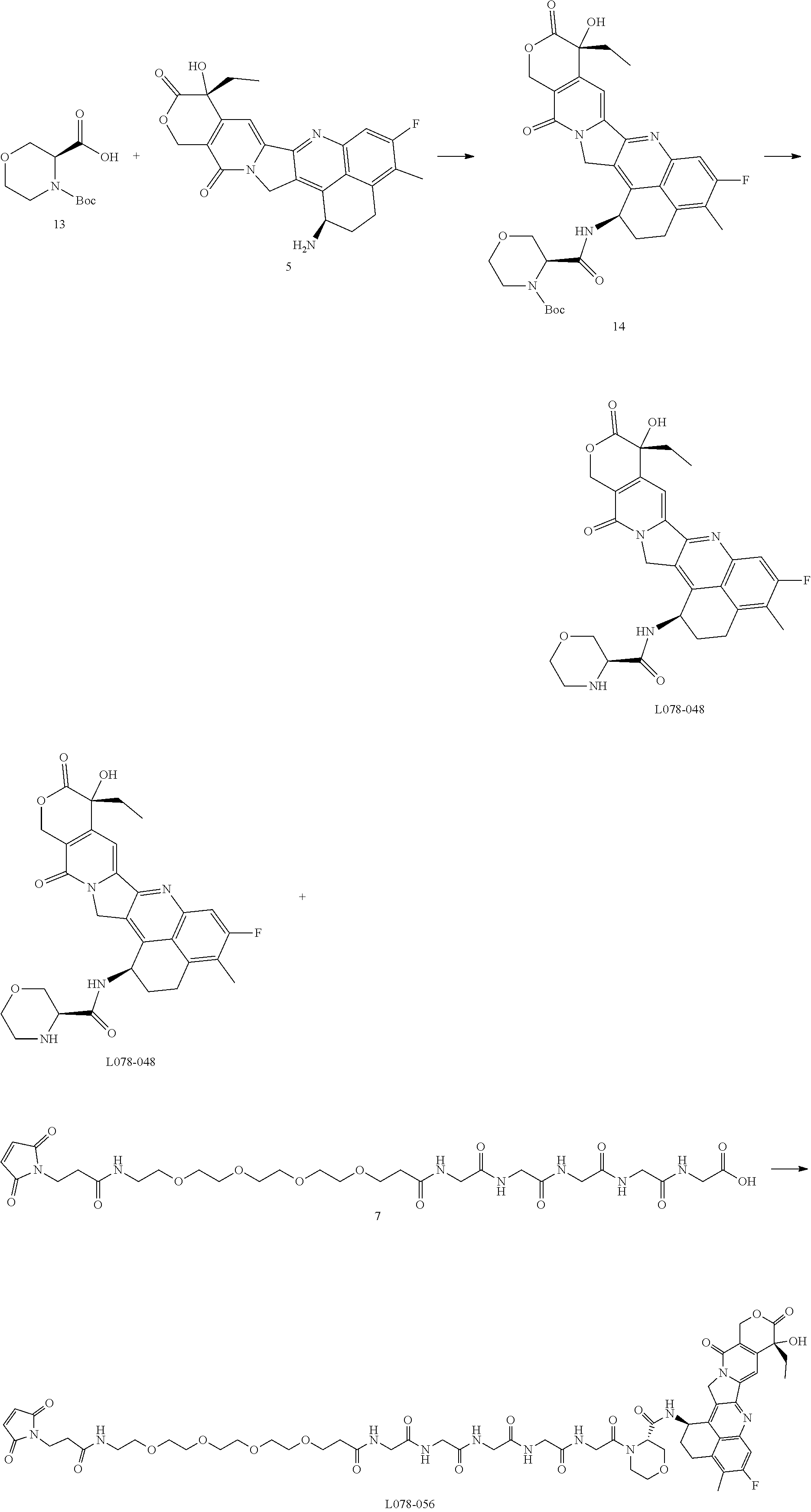

To a solution of compounds 13 (22 mg, 94 μmol), 2 (Mesylate salt, 50 mg, 94 μmol) and HATU (36 mg, 94 μmol) in 2 mL of DMF was added DIEA (30 mg, 235 μmol). The solution was stirred for 10 minutes and purified by HPLC. The resulting product 14 was treated with 50% TFA/DCM (2 mL) for 30 minutes and purified by HPLC to give compound L078-048 (48 mg). MS m/z 549.8 (M+H).

To a solution of compounds 4 (10 mg, 14 μmol), L078-048 (TFA salt, 9.5 mg, 14 μmol) and HATU (5 mg, 14 μmol) in 2 mL of DMF was added DIEA (5 mg, 35 μmol). The solution was stirred for 5 minutes and purified by HPLC to give compound L078-056 (2.1 mg) as a yellow powder. MS m/z 1232.6 (M+H).

### Example S5: Synthesis of Compound L078-078

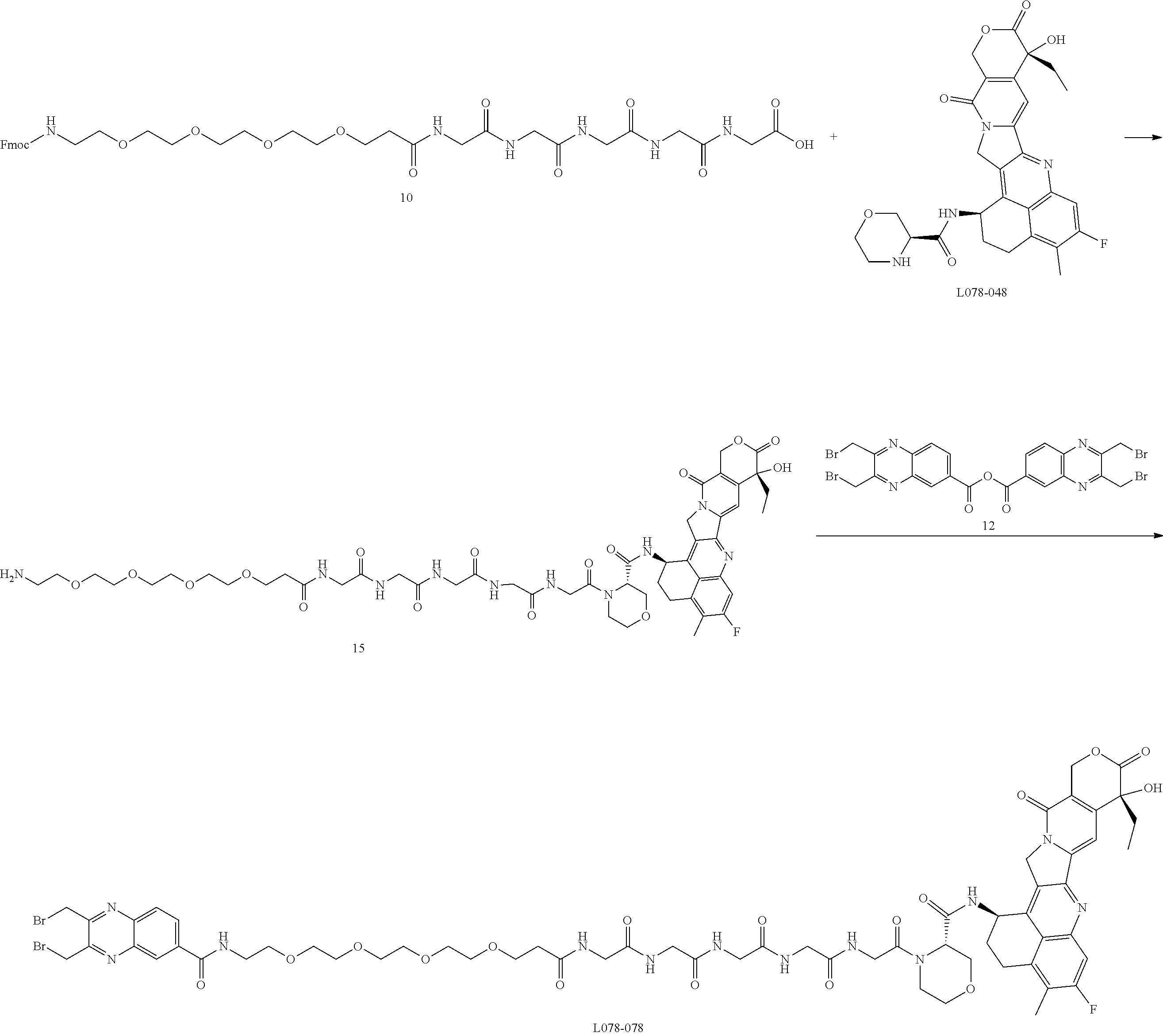

A suspension of 10 (40 mg, 45 μmol) in DMSO (6 mL) was heated to 60° C. The clear solution was cooled to room temperature. To the solution was added L078-048 (TFA salt, 30 mg, 45 μmol), HATU (17 mg, 45 μmol) and DIEA (15 mg, 112 μmol). The solution was stirred for 5 minutes followed by addition of 1 mL diethylamine, stirred for additional 30 minutes and concentrated. The residue was purified by HPLC to give 15 as a TFA salt. MS m/z 1081.8 (M+H).

A solution of compound 15 (68 mg, 57 μmol), 12 (40 mg, 57 μmol) and DIEA (15 mg, 112 μmol) was stirred for 5 minutes and purified by HPLC to give compound L078-078 (16.1 mg). MS m/z 1423.5 (M+H).

### Example S6: Synthesis of Compound L078-059

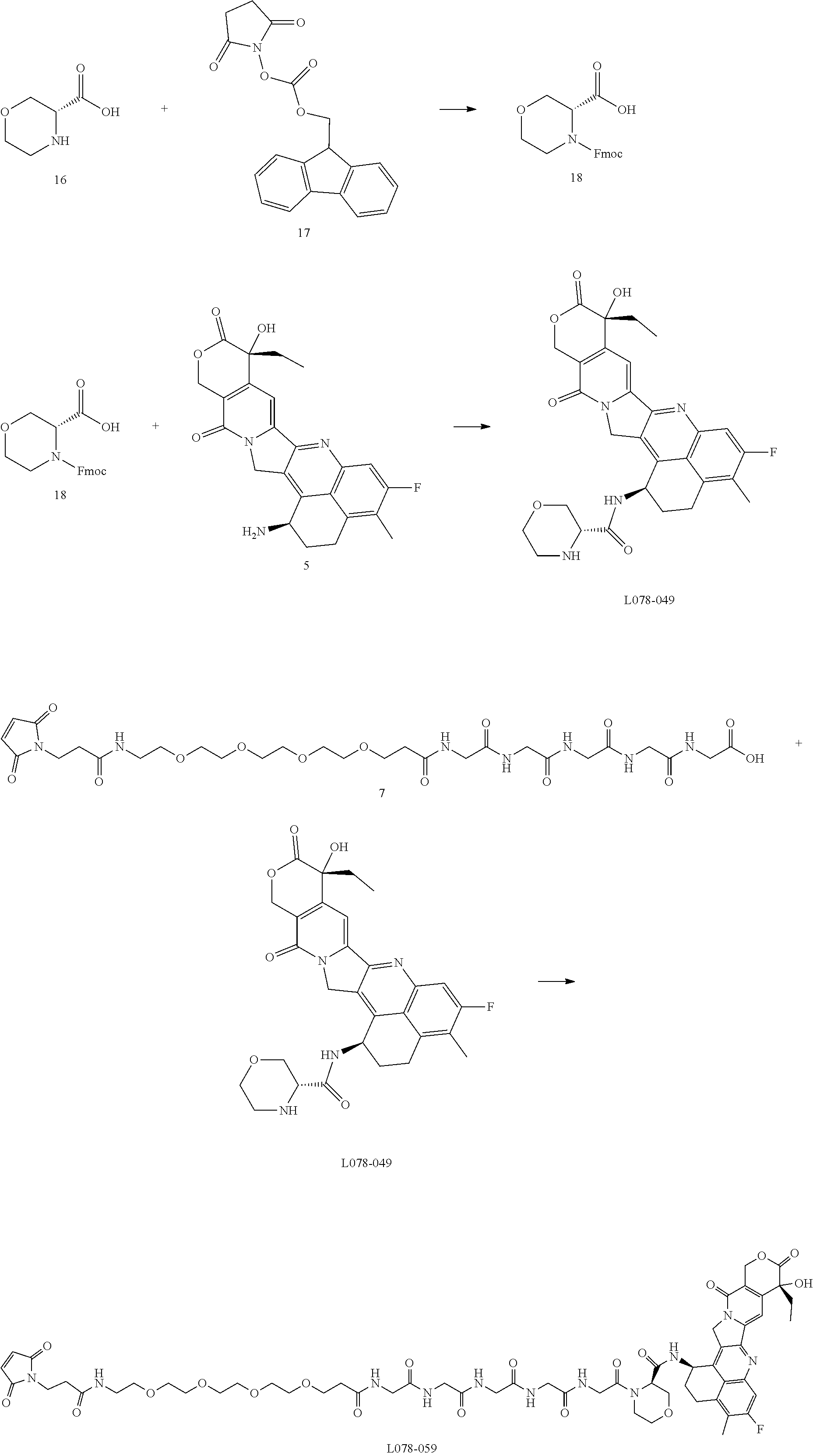

To a solution of compounds 16 (5 mg, 38 μmol) and 17 (13 mg, 38 μmol) in 5 mL of 50% CH₃CN/H₂O added 0.5 mL of Sat. NaHCO₃ solution. The solution was stirred for 10 minutes. The mixture was purified by HPLC to give compound 18 (13 mg).

To a solution of compounds 18 (13 mg, 38 μmol), 5 (Mesylate salt, 20 mg, 38 μmol) and HATU (15 mg, 38 μmol) in 2 mL of DMF was added DIEA (12 mg, 95 mol). The solution was stirred for 5 minutes followed by addition of 0.5 mL diethylamine. The solution was stirred for additional 30 minutes and diethylamine was removed by evaporation under reduced pressure. The residue was purified by HPLC to give compound L078-049 (15 mg, 22 μmol) as a TFA salt.

To a solution of compounds L078-049 (TFA salt, 15 mg, 22 μmol), 7 (16 mg, 22 μmol) and HATU (8.5 mg, 22 μmol) in 2 mL of DMF was added DIEA (7 mg, 55 μmol). The solution was stirred for 5 minutes and purified by HPLC to give compound L078-059 (6.5 mg) as a yellow powder. MS m/z 1232.5 (M+H).

### Example S7: Synthesis of Compound L078-055

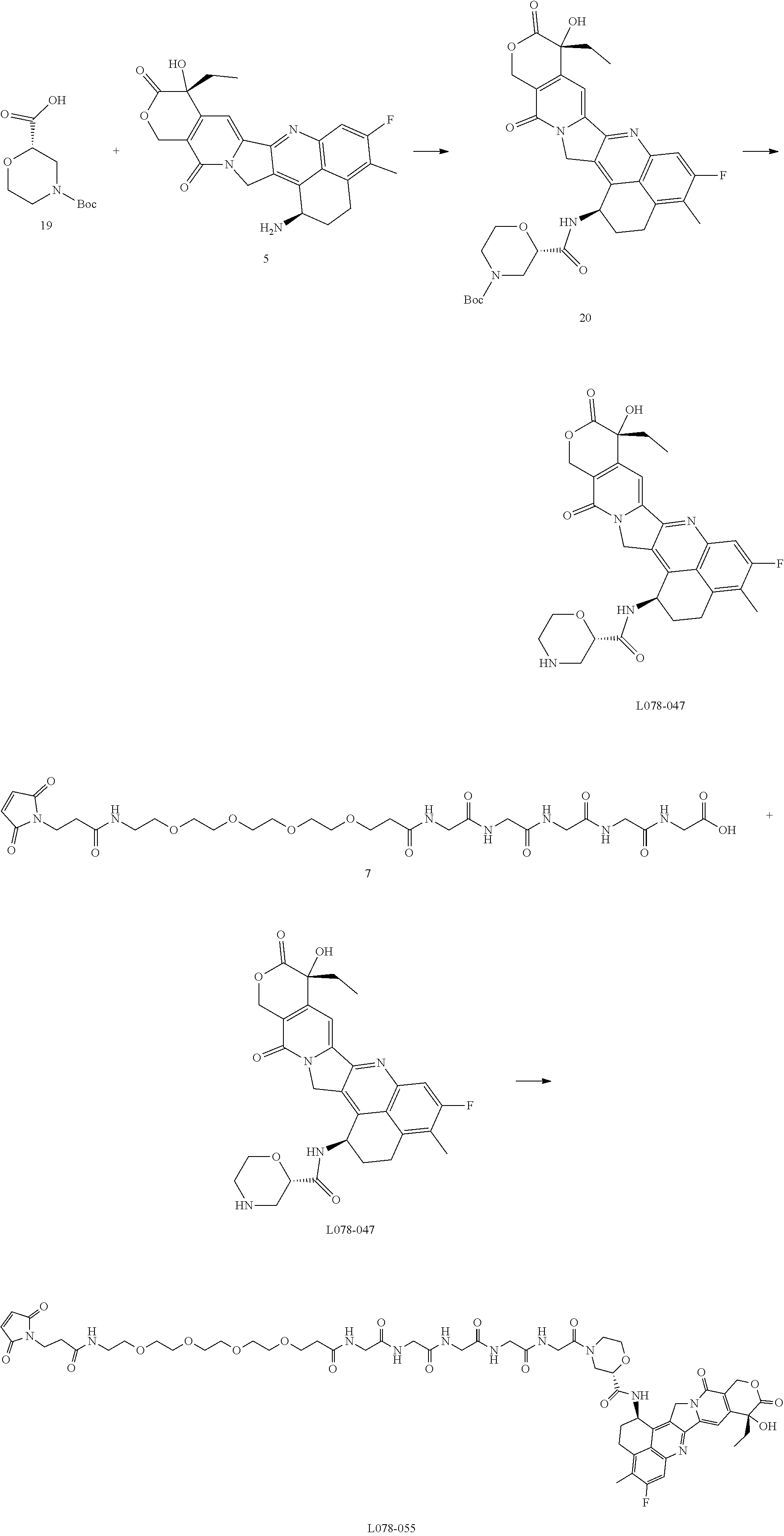

To a solution of compounds 19 (5 mg, 19 μmol), 5 (Mesylate salt, 10 mg, 19 μmol) and HATU (7 mg, 19 μmol) in 2 mL of DMF was added DIEA (6 mg, 48 μmol). The solution was stirred for 10 minutes and purified by HPLC. The resulting product 20 was treated with 50% TFA/DCM (2 mL) for 30 minutes and purified by HPLC to give compound L078-047 (9 mg) as a TFA salt. MS m/z 549.4 (M+H).

To a solution of compounds 7 (9.5 mg, 14 μmol), L078-047 (TFA salt, 9 mg, 14 μmol) and HATU (5 mg, 14 μmol) in 2 mL of DMF was added DIEA (5 mg, 35 μmol). The solution was stirred for 5 minutes and purified by HPLC to give compound L078-055 (2.6 mg) as a yellow powder. MS m/z 1232.5 (M+H).

### Example S8: Synthesis of Compound L078-058

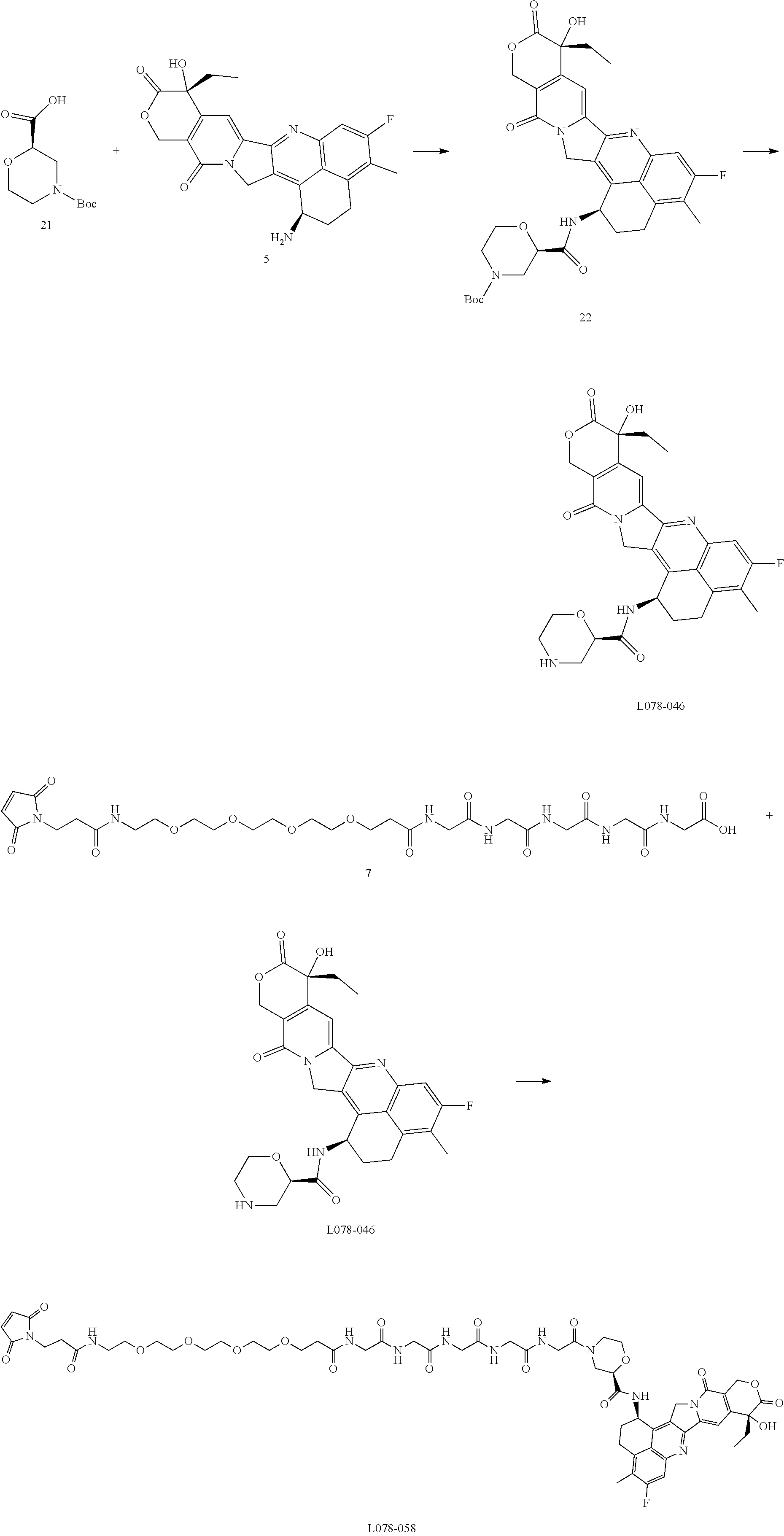

To a solution of compounds 21 (5 mg, 19 μmol), 5 (mesylate salt, 10 mg, 19 μmol) and HATU (7 mg, 19 μmol) in 2 mL of DMF was added DIEA (6 mg, 48 μmol). The solution was stirred for 5 minutes and purified by HPLC. The resulting product 22 was treated with 50% TFA in DCM (1 mL) for 30 minutes and concentrated to give compound L078-046 (9.6 mg) as a yellow powder. MS m/z 549.6 (M+H).

To a solution of compounds 7 (9.5 mg, 14 μmol), L078-046 (TFA salt, 9 mg, 14 μmol) and HATU (5 mg, 14 μmol) in 2 mL of DMF was added DIEA (5 mg, 35 μmol). The solution was stirred for 5 minutes and purified by HPLC to give compound L078-058 (5 mg) as a yellow powder. MS m/z 1232.5 (M+H).

### Example S9: Synthesis of Compound L078-063

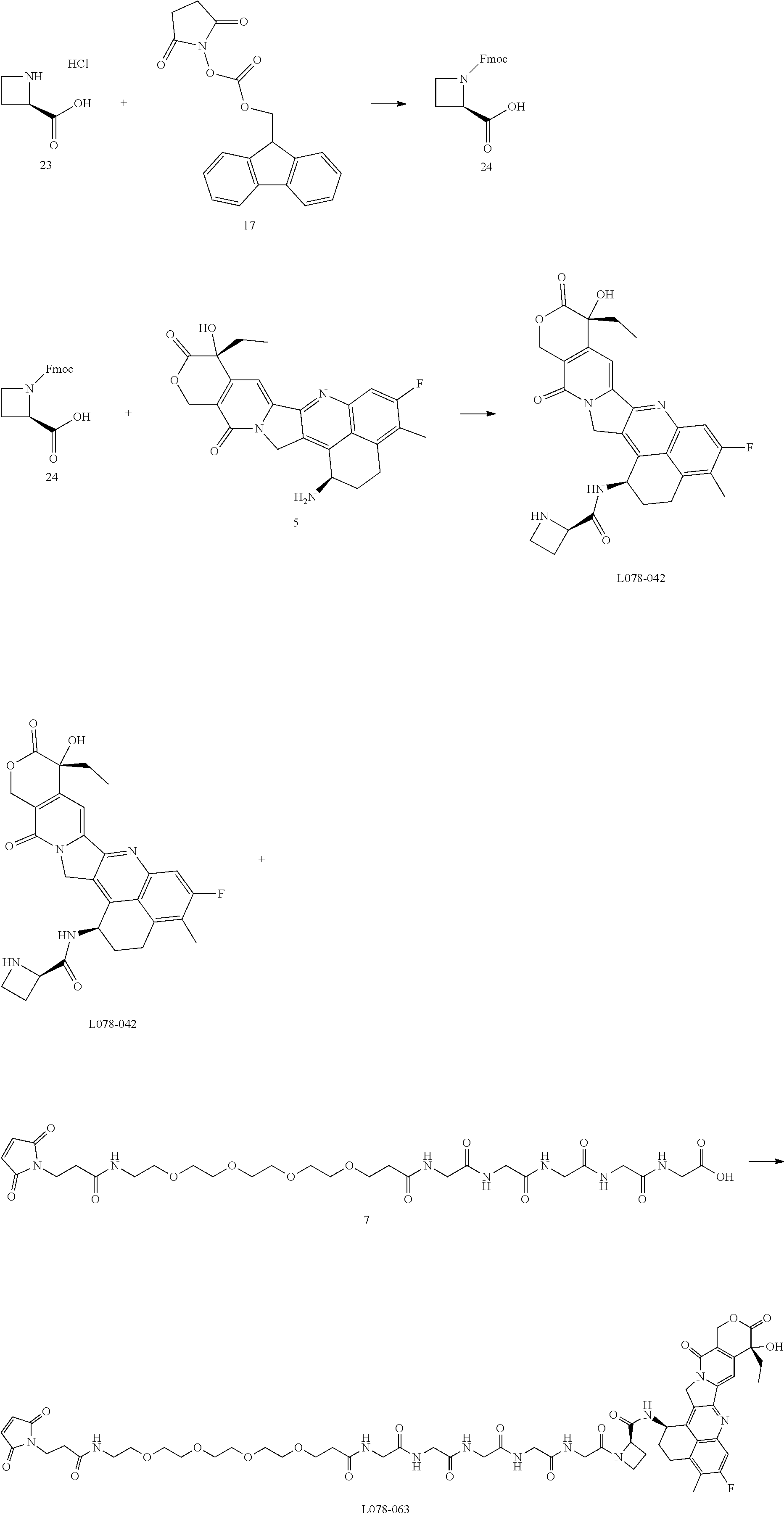

To a solution of compounds 23 (HCl salt, 50 mg, 0.36 mmol) and 17 (122 mg, 0.36 mmol) in 4 mL of 50% CH₃CN/H₂O added 0.5 mL of Sat. NaHCO₃ solution. The solution was stirred for 10 minutes. The mixture was purified by HPLC to give compound 24 (82 mg).

To a solution of compounds 24 (30 mg, 0.094 mmol), 5 (mesylate salt, 50 mg, 0.094 mmol) and HATU (36 mg, 0.094 mmol) in DMF (3 mL) was added DIEA (30 mg, 0.235 mmol). After being stirred for 5 minutes to the solution was added 1 mL of diethylamine. The reaction mixture was stirred for another 30 minutes. Diethylamine was removed by evaporation under reduced pressure. The residue was purified by HPLC to give compound L078-042 as a TFA salt (41 mg). MS m/z 519.3 (M+H).

To a solution of compounds L078-042 (TFA salt, 9 mg, 14 μmol), 7 (10 mg, 14 μmol) and HATU (6 mg, 14 μmol) in 2 mL of DMF was added DIEA (5 mg, 38 μmol). The solution was stirred for 5 minutes and purified by HPLC to give compound L078-063 (6 mg) as a yellow powder. MS m/z 1203.1 (M+H).

### Example S10: Synthesis of Compound L078-064

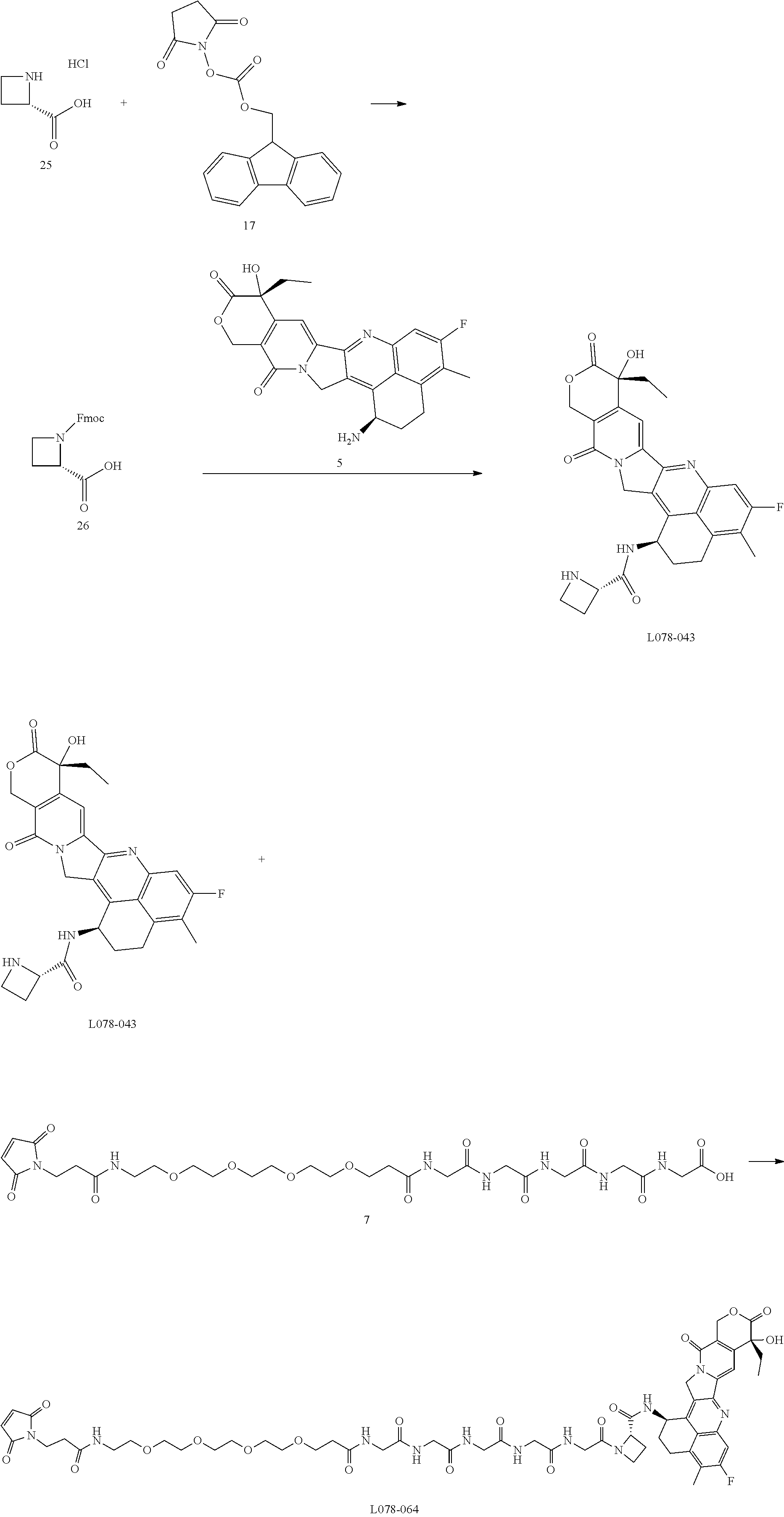

To a solution of compounds 25 (HCl salt, 50 mg, 0.36 mmol) and 17 (122 mg, 0.36 mmol) in 4 mL of 50% CH₃CN/H₂O added 0.5 mL of Sat. NaHCO₃ solution. The solution was stirred for 10 minutes. The mixture was purified by HPLC to give compound 26 (102 mg).

To a solution of compounds 26 (20 mg, 0.060 mmol), 5 (mesylate salt, 33 mg, 0.060 mmol) and HATU (23 mg, 0.060 mmol) in DMF (3 mL) was added DIEA (20 mg, 0.15 mmol). After being stirred for 5 minutes to the solution was added 1 mL of diethylamine. The reaction mixture was stirred for another 30 minutes. Diethylamine was removed by evaporation under reduced pressure. The residue was purified by HPLC to give compound L078-043 as a TFA salt (28 mg). MS m/z 519.3 (M+H).

To a solution of compounds L078-043 (TFA salt, 9 mg, 14 μmol), 7 (10 mg, 14 μmol) and HATU (6 mg, 14 μmol) in 2 mL of DMF was added DIEA (5 mg, 38 μmol). The solution was stirred for 5 minutes and purified by HPLC to give compound L078-064 (1.5 mg) as a yellow powder. MS m/z 1202.9 (M+H).

### Example S11: Synthesis of Compound L078-066LT

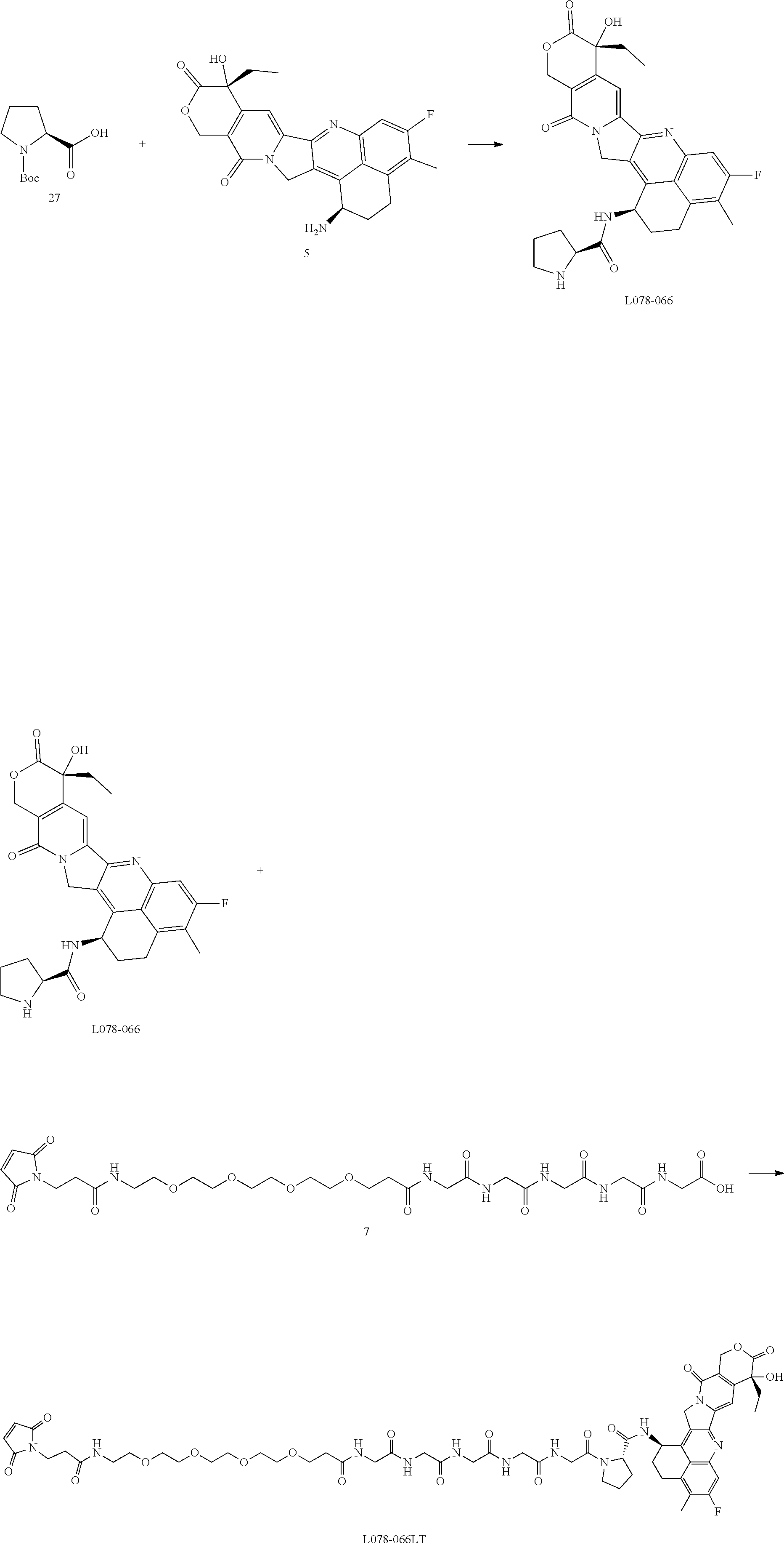

To a solution of compounds 27 (14 mg, 55 μmol), 5 (Mesylate salt, 30 mg, 55 μmol) and HATU (21 mg, 55 μmol) in 2 mL of DMF was added DIEA (11 mg, 83 μmol). The solution was stirred for 10 minutes and purified by HPLC. The resulting product (29 mg) was treated with 50% TFA/DCM (2 mL) for 30 minutes and purified by HPLC to give compound L078-066 as a TFA salt. MS m/z 532.7 (M+H).

A suspension of compound 7 (20 mg, 28 μmol) in DMSO (3 mL) was heated to 60 C. The clear solution was cooled to r.t. To the solution was added L078-066 (TFA salt, 18 mg, 28 μmol), HATU (11 mg, 28 μmol) and DIEA (9 mg, 70 mol). The solution was stirred for 5 minutes and purified by HPLC to give compound L078-066LT (15 mg) as a yellow powder. MS m/z 1217.1 (M+H).

### Example S12: Synthesis of Compound L078-065LT

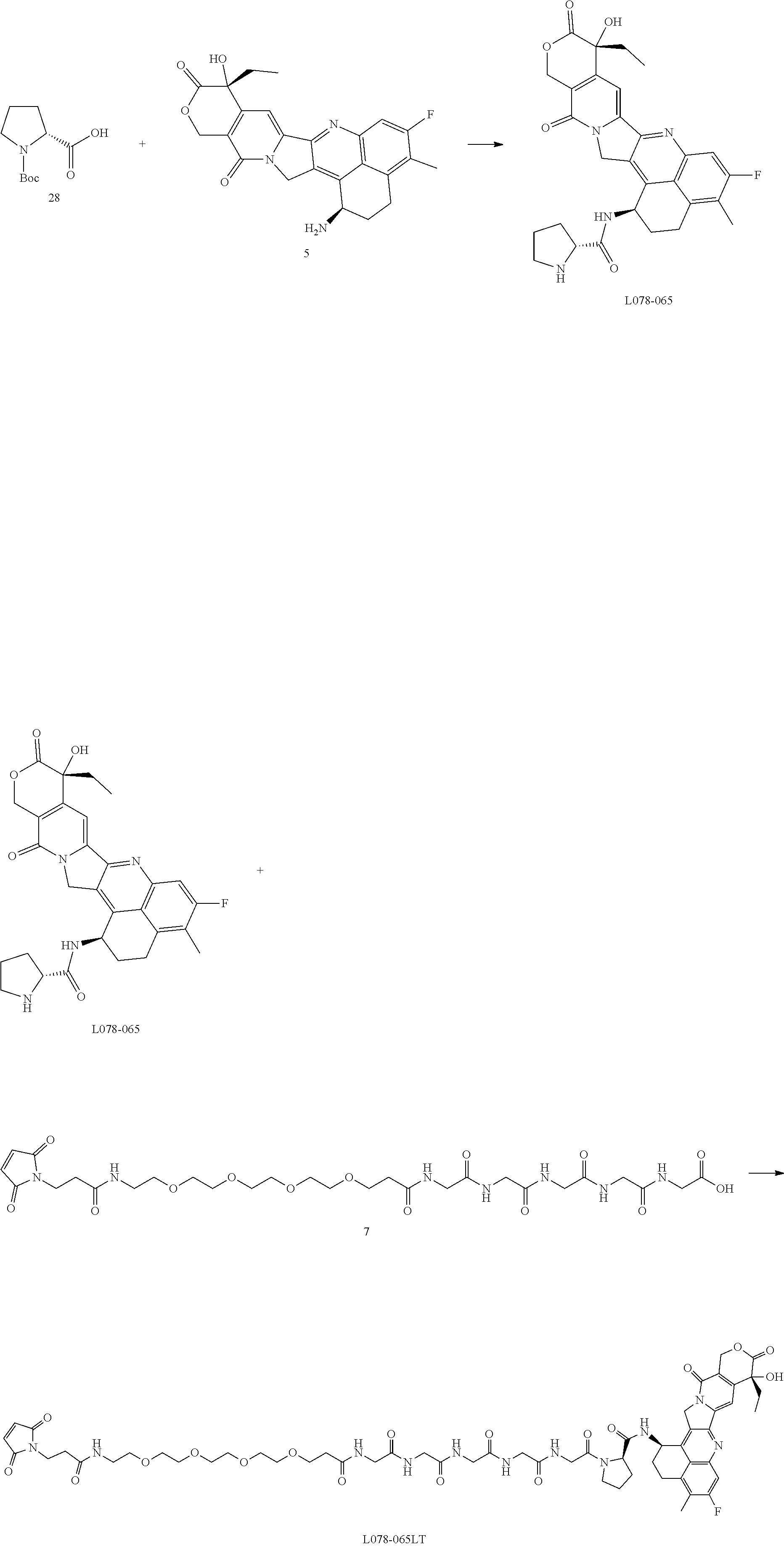

To a solution of compounds 28 (14 mg, 55 μmol), 5 (Mesylate salt, 30 mg, 55 μmol) and HATU (21 mg, 55 μmol) in 2 mL of DMF was added DIEA (11 mg, 83 μmol). The solution was stirred for 10 minutes and purified by HPLC. The resulting product (29 mg) was treated with 50% TFA/DCM (2 mL) for 30 minutes and purified by HPLC to give compound L078-065 (23 mg) as a TFA salt. MS m/z 532.9 (M+H).

A suspension of compound 7 (20 mg, 28 μmol) in DMSO (3 mL) was heated to 60 C. The clear solution was cooled to r.t. To the solution was added L078-065 (TFA salt, 18 mg, 28 μmol), HATU (12 mg, 28 μmol) and DIEA (9 mg, 70 mol). The solution was stirred for 5 minutes and purified by HPLC to give compound L078-065LT (14 mg) as a yellow powder. MS m/z 1217.0 (M+H).

### Example S13: Synthesis of Compound L078-057

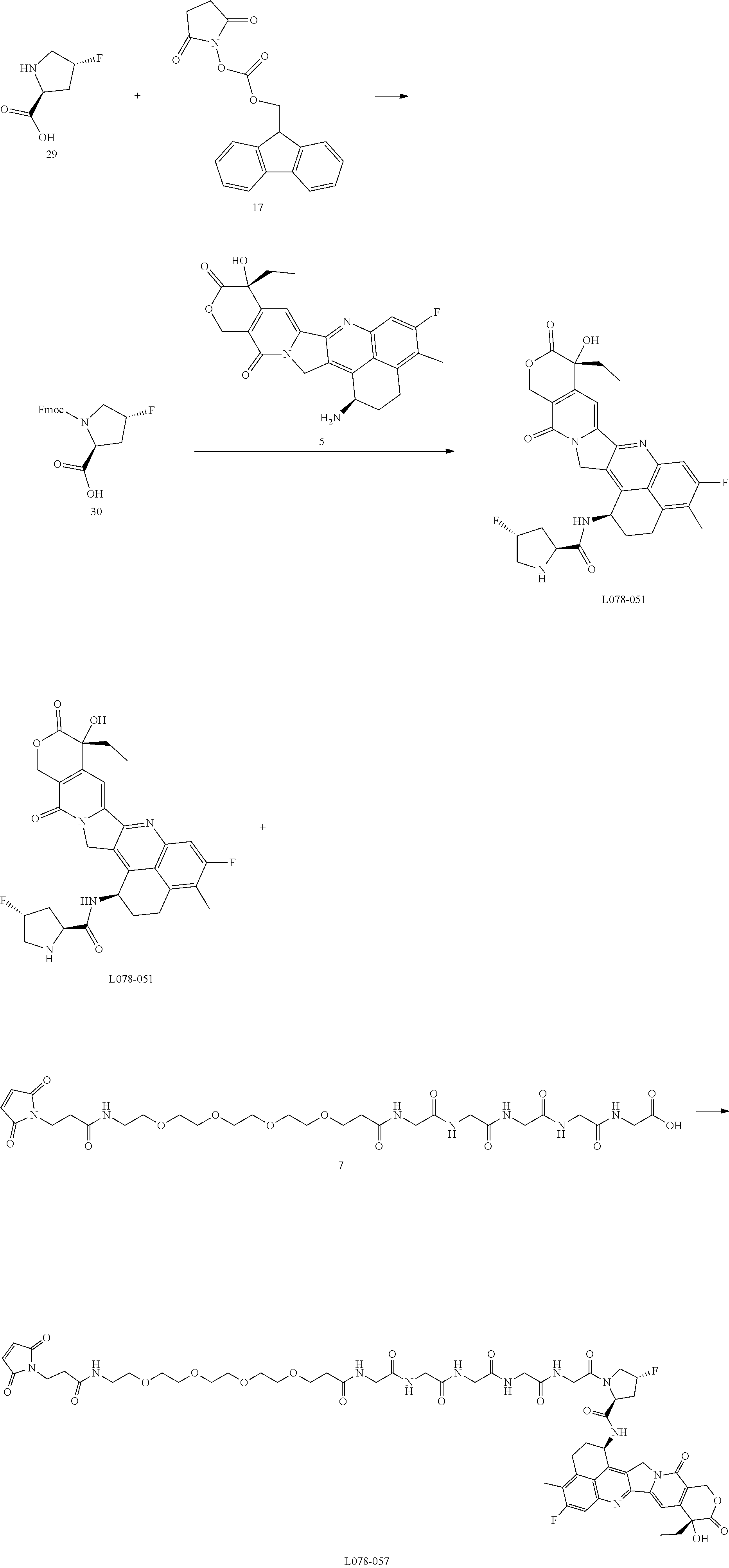

To a solution of compounds 29 (5 mg, 38 μmol) and 17 (13 mg 20 mg, 28 μmol) in 4 mL of 50% CH₃CN/H₂O added 0.5 mL of Sat. NaHCO₃ solution. The solution was stirred for 20 minutes. The solution was extracted with dichloromethane, dried, and concentrated to give compound 30 (12 mg). MS m/z 356.4 (M+H).

To a solution of compounds 30 (12 mg, 34 μmol), 5 (mesylate salt, 20 mg, 38 μmol) and HATU (14 mg, 34 μmol) in DMF (2 mL) was added DIEA (11 mg, 85 μmol). After being stirred for 5 minutes to the solution was added 0.5 mL of diethylamine. The reaction mixture was stirred for another 30 minutes. Diethylamine was removed by evaporation under reduced pressure. The residue was purified by HPLC to give compound L078-051 as a TFA salt (18 mg). MS m/z 551.1 (M+H).

To a solution of compounds L078-051 (TFA salt, 10 mg, 14 μmol), 7 (10 mg, 20 mg, 28 μmol) and HATU (6 mg, 14 μmol) in 2 mL of DMF was added DIEA (5 mg, 38 μmol). The solution was stirred for 5 minutes and purified by HPLC to give compound L078-057 (3.1 mg) as a yellow powder. MS m/z 1234.4 (M+H).

### Example S14: Synthesis of Compound L078-045

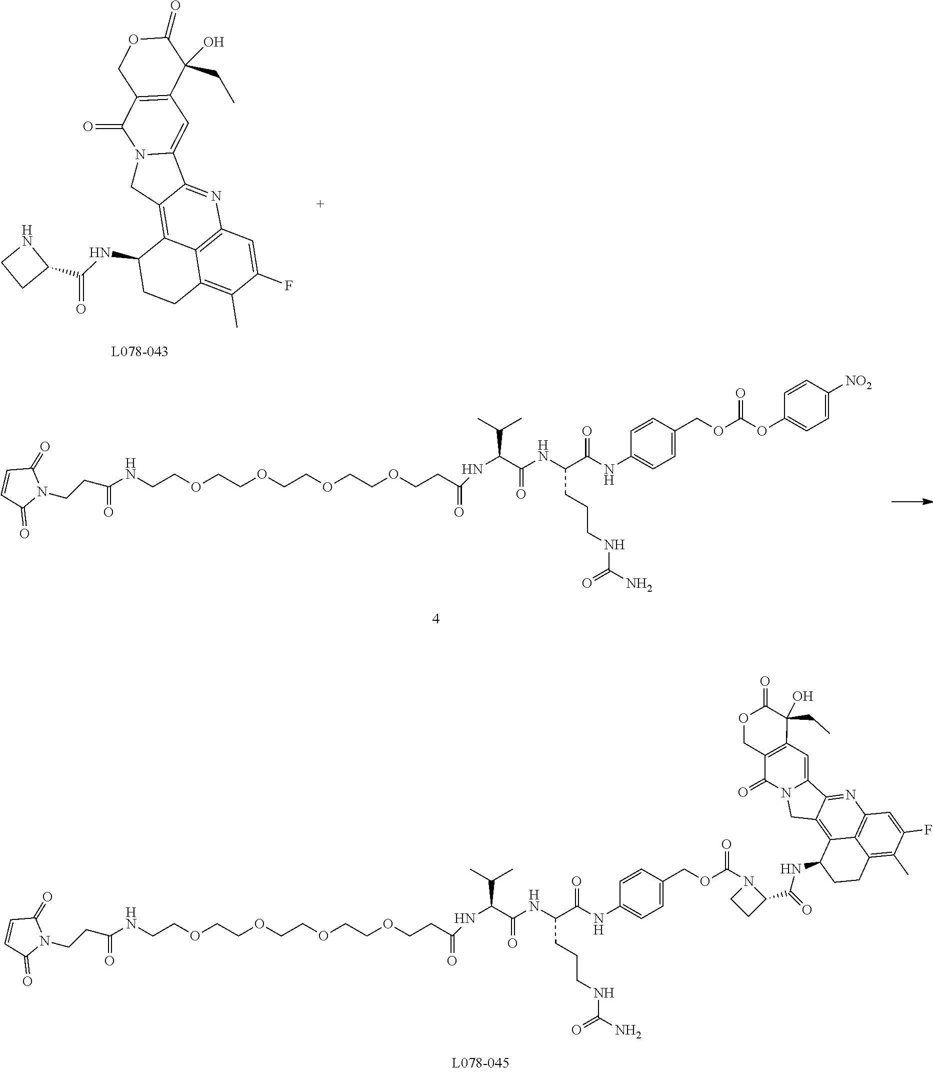 [SMILES: CC[C@]1(O)C(=O)OCc2c1cc1n(c2=O)Cc2c-1nc1cc(F)c(C)c3c1c2[C@H](NC(=O)[C@@H]1CCN1)CC3 ; CC(C)[C@H](NC(=O)CCOCCOCCOCCOCCNC(=O)CCN1C(=O)C=CC1=O)C(=O)N[C@@H](CCCNC(N)=O)C(=O)Nc1ccc(COC(=O)Oc2ccc([N+](=O)[O-])cc2)cc1 ; CC[C@]1(O)C(=O)OCc2c1cc1n(c2=O)Cc2c-1nc1cc(F)c(C)c3c1c2[C@H](NC(=O)[C@@H]1CCN1C(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1)CC3]

A solution of compound L078-043 (TFA salt, 20 mg, 30 μmol), 4 (30 mg, 32 μmol), HOBt (2 mg, 14 μmol) and DIEA (7 mg, 54 μmol) in 2 mL of DMF was stirred for one hour. The residue was purified by HPLC to give compound L078-045 (14 mg) as a yellow powder. MS m/z 1323.0 (M+H).

### Example S15: Synthesis of Compound L078-044

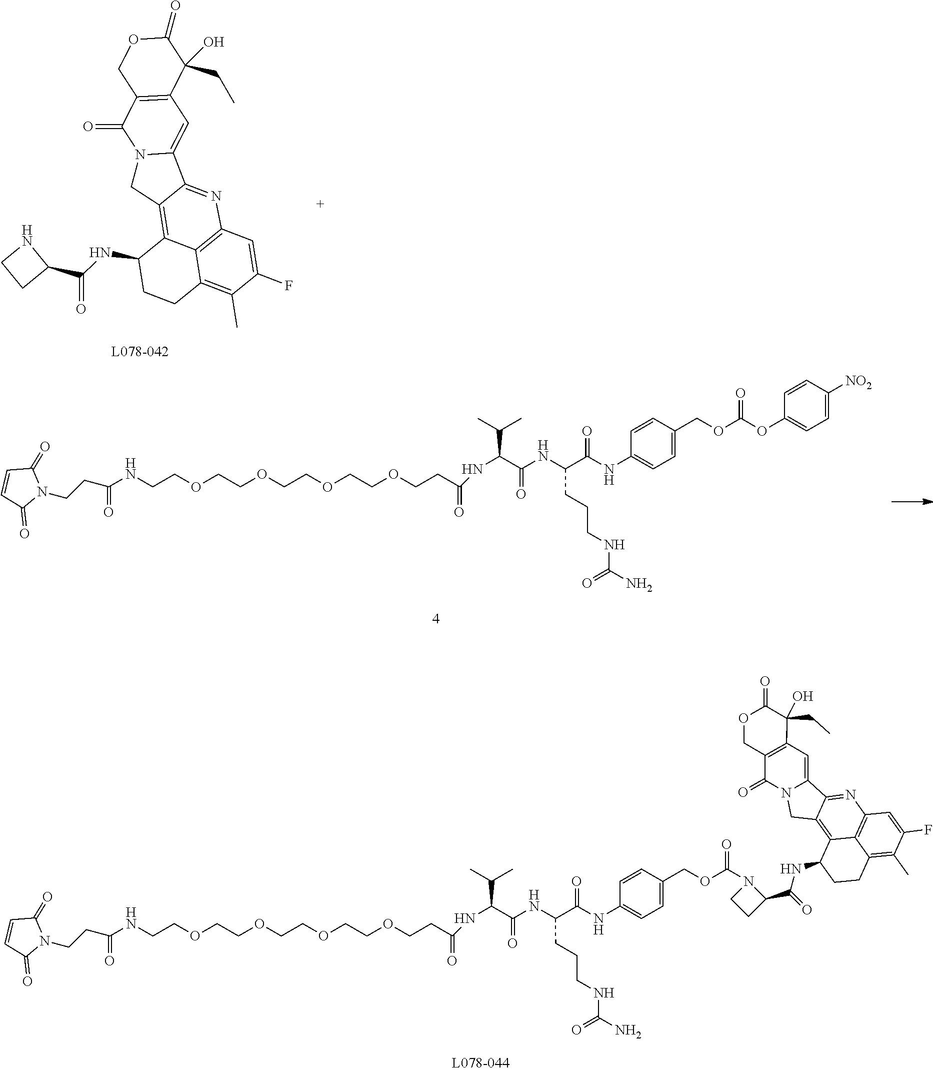 [SMILES: CC[C@]1(O)C(=O)OCc2c1cc1n(c2=O)Cc2c-1nc1cc(F)c(C)c3c1c2[C@H](NC(=O)[C@H]1CCN1)CC3 ; CC(C)[C@H](NC(=O)CCOCCOCCOCCOCCNC(=O)CCN1C(=O)C=CC1=O)C(=O)N[C@@H](CCCNC(N)=O)C(=O)Nc1ccc(COC(=O)Oc2ccc([N+](=O)[O-])cc2)cc1 ; CC[C@]1(O)C(=O)OCc2c1cc1n(c2=O)Cc2c-1nc1cc(F)c(C)c3c1c2[C@H](NC(=O)[C@H]1CCN1C(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1)CC3]

A solution of compound L078-042 (TFA salt, 17 mg, 27 μmol), 4 (25 mg, 27 μmol), HOBt (2 mg, 14 μmol), and DIEA (7 mg, 54 μmol) in 2 mL of DMF was stirred for one hour. The residue was purified by HPLC to give compound L-78-044 (16 mg) as a yellow powder. MS m/z 1323.0 (M+H).

### Example S16: Synthesis of Compound L078-081-LT

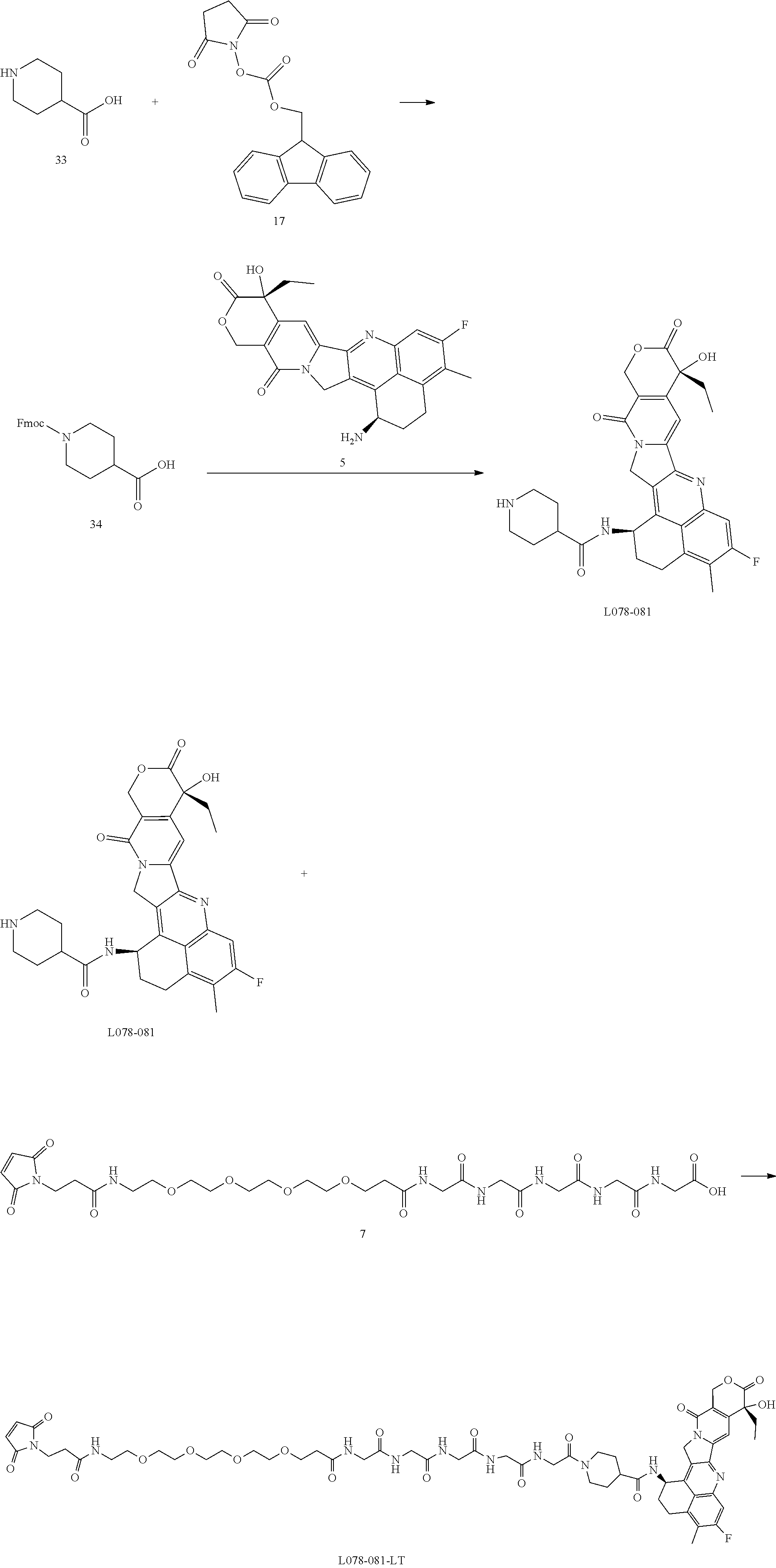

To a solution of compounds 33 (50 mg, 0.388 mmol) and 17 (131 mg, 0.388 mmol) in 4 mL of 50% CH₃CN/H₂O added 0.5 mL of Sat. NaHCO₃ solution. The solution was stirred for 20 minutes. The solution was purified by HPLC to give compound 34 (56 mg). MS m/z 352.5 (M+H).

To a solution of compounds 34 (50 mg, 142 μmol), 5 (mesylate salt, 75 mg, 142 μmol) and HATU (54 mg, 142 μmol) in DMF (2 mL) was added DIEA (46 mg, 355 μmol). After being stirred for 5 minutes to the solution was added 0.5 mL of diethylamine. The reaction mixture was stirred for another 30 minutes. Diethylamine was removed by evaporation under reduced pressure. The residue was purified by HPLC to give compound L078-081 as a TFA salt (51 mg). MS m/z 547.3 (M+H).

To a solution of compounds L078-081 (TFA salt, 10 mg, 15 μmol), 7 (10 mg, 15 μmol) and HATU (6 mg, 15 μmol) in 2 mL of DMF was added DIEA (5 mg, 38 μmol). The solution was stirred for 5 minutes and purified by HPLC to give compound L078-081-LT (2.0 mg) as a yellow powder. MS m/z 1230.6 (M+H).

### Example S17: Synthesis of Compound L078-090

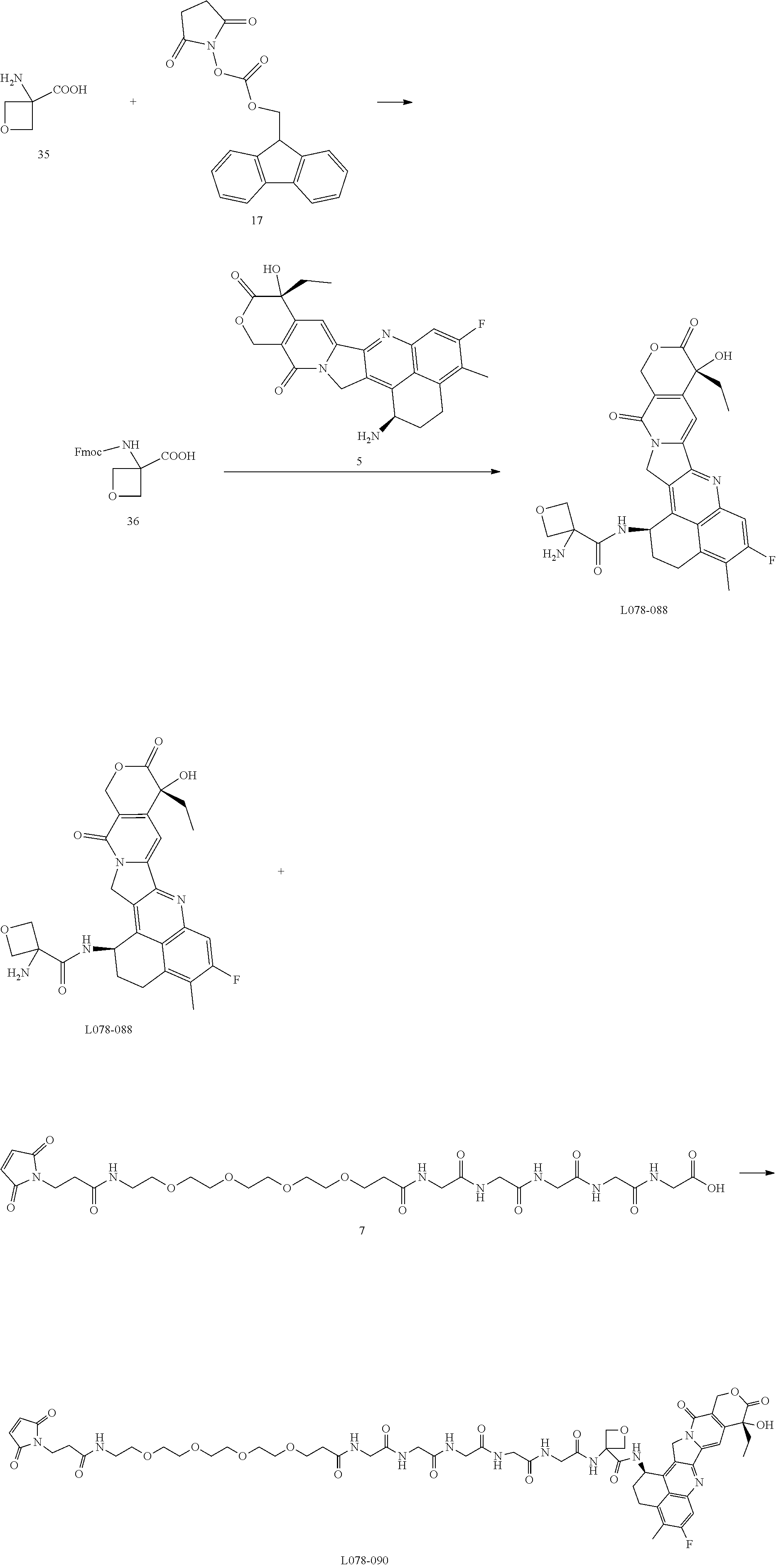

To a solution of compounds 35 (250 mg, 2.13 mmol) and 17 (720 mg, 2.13 mmol) in 10 mL of 50% CH₃CN/H₂O added DIEA (412 mg, 3.2 mmol). The solution was stirred for 10 minutes. The mixture was purified by HPLC to give compound 36 (451 mg).

To a solution of compounds 36 (30 mg, 88 μmol), 5 (mesylate salt, 47 mg, 88 μmol) and HATU (33 mg, 88 μmol) in DMF (2 mL) was added DIEA (28 mg, 220 μmol). After being stirred for 5 minutes to the solution was added 0.5 mL of diethylamine. The reaction mixture was stirred for another 30 minutes. Diethylamine was removed by evaporation under reduced pressure. The residue was purified by HPLC to give compound L078-088 as a TFA salt (36 mg). MS m/z 532.2 (M+H).

To a solution of compounds L078-088 (TFA salt, 15 mg, 23 μmol), 7 (16 mg, 23 μmol) and HATU (9 mg, 23 μmol) in 2 mL of DMF was added DIEA (7 mg, 58 μmol). The solution was stirred for 5 minutes and purified by HPLC to give compound L078-090 (5.2 mg) as a yellow powder. MS m/z 1219.1 (M+H).

### Example S18: Synthesis of Compound L078-091

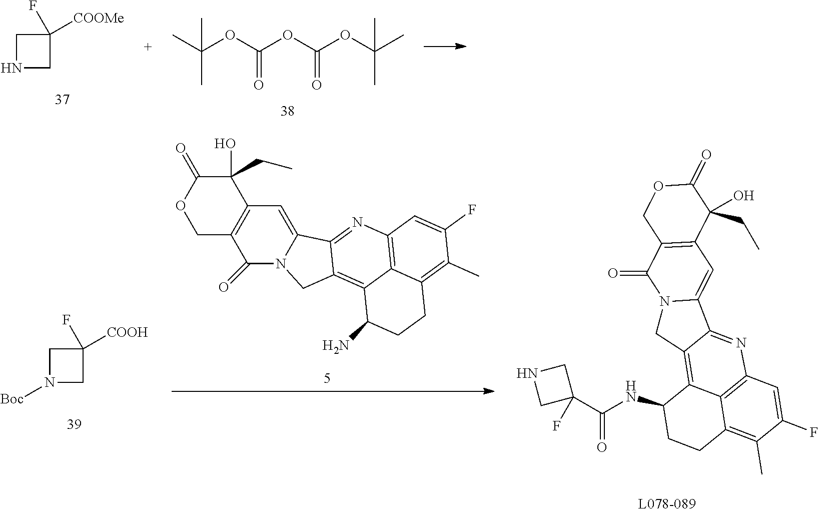 [SMILES: COC(=O)C1(F)CNC1 ; CC(C)(C)OC(=O)OC(=O)OC(C)(C)C ; CC(C)(C)OC(=O)N1CC(F)(C(=O)O)C1 ; CC[C@]1(O)C(=O)OCc2c1cc1n(c2=O)Cc2c-1nc1cc(F)c(C)c3c1c2[C@H](N)CC3 ; CC[C@]1(O)C(=O)OCc2c1cc1n(c2=O)Cc2c-1nc1cc(F)c(C)c3c1c2[C@H](NC(=O)C1(F)CNC1)CC3]

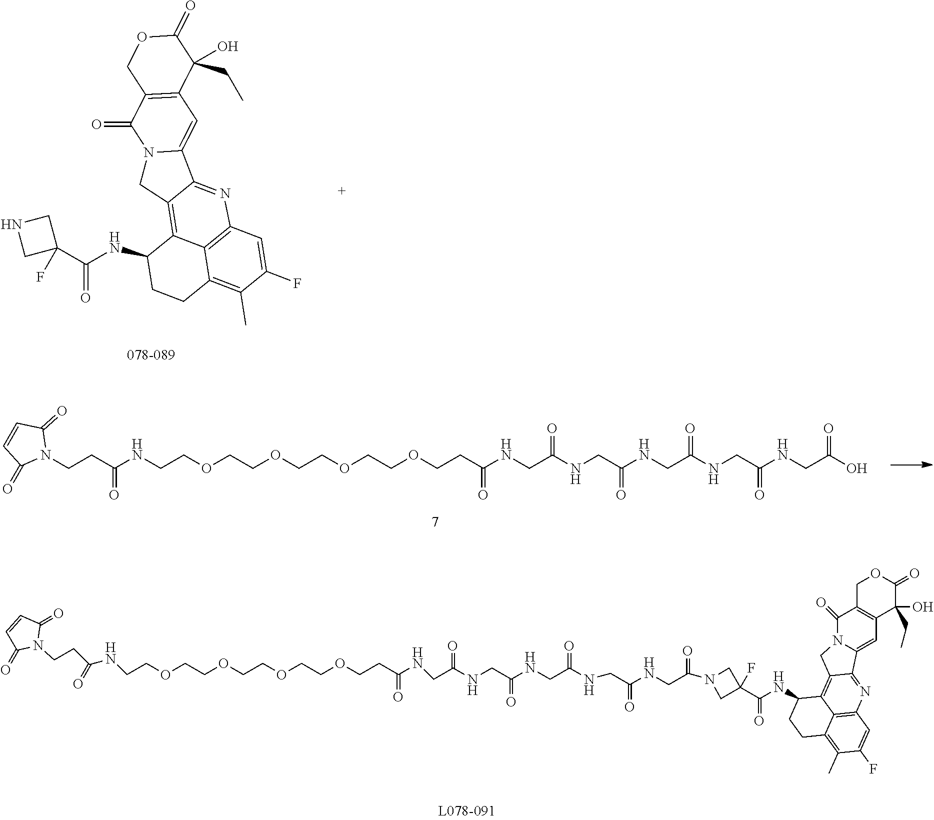 [SMILES: CC[C@]1(O)C(=O)OCc2c1cc1n(c2=O)Cc2c-1nc1cc(F)c(C)c3c1c2[C@H](NC(=O)C1(F)CNC1)CC3 ; O=C(O)CNC(=O)CNC(=O)CNC(=O)CNC(=O)CNC(=O)CCOCCOCCOCCOCCNC(=O)CCN1C(=O)C=CC1=O ; CC[C@]1(O)C(=O)OCc2c1cc1n(c2=O)Cc2c-1nc1cc(F)c(C)c3c1c2[C@H](NC(=O)C1(F)CN(C(=O)CNC(=O)CNC(=O)CNC(=O)CNC(=O)CNC(=O)CCOCCOCCOCCOCCNC(=O)CCN2C(=O)C=CC2=O)C1)CC3]

A solution of compounds 37 (HCl salt, 250 mg, 1.48 mmol), 38 (643 mg, 2.95 mmol) and NaOH (296 mg, 7.4 mmol) in MeOH (10 mL)/H₂O (1 mL) was stirred for 5 hours. The mixture was purified by HPLC to give compound 39 (305 mg).

To a solution of compounds 39 (21 mg, 94 μmol), 5 (mesylate salt, 50 mg, 94 μmol) and HATU (36 mg, 94 μmol) in DMF (2 mL) was added DIEA (30 mg, 235 μmol). After being stirred for 5 minutes the solution was purified by HPLC and concentrated to dry. The residue was treated with 50% TFA in dichloromethane (2 mL) for 30 minutes to give compound L078-089 (TFA salt, 52 mg). MS m/z 537.3 (M+H).

To a solution of compounds L078-089 (TFA salt, 15 mg, 23 μmol), 7 (16 mg, 23p mol) and HATU (9 mg, 23 μmol) in 2 mL of DMF was added DIEA (7 mg, 58 μmol). The solution was stirred for 5 minutes and purified by HPLC to give compound L078-091 (9.2 mg) as a pale-yellow powder. MS m/z 1221.3 (M+H).

### Example S19: Synthesis of Compound L078-084

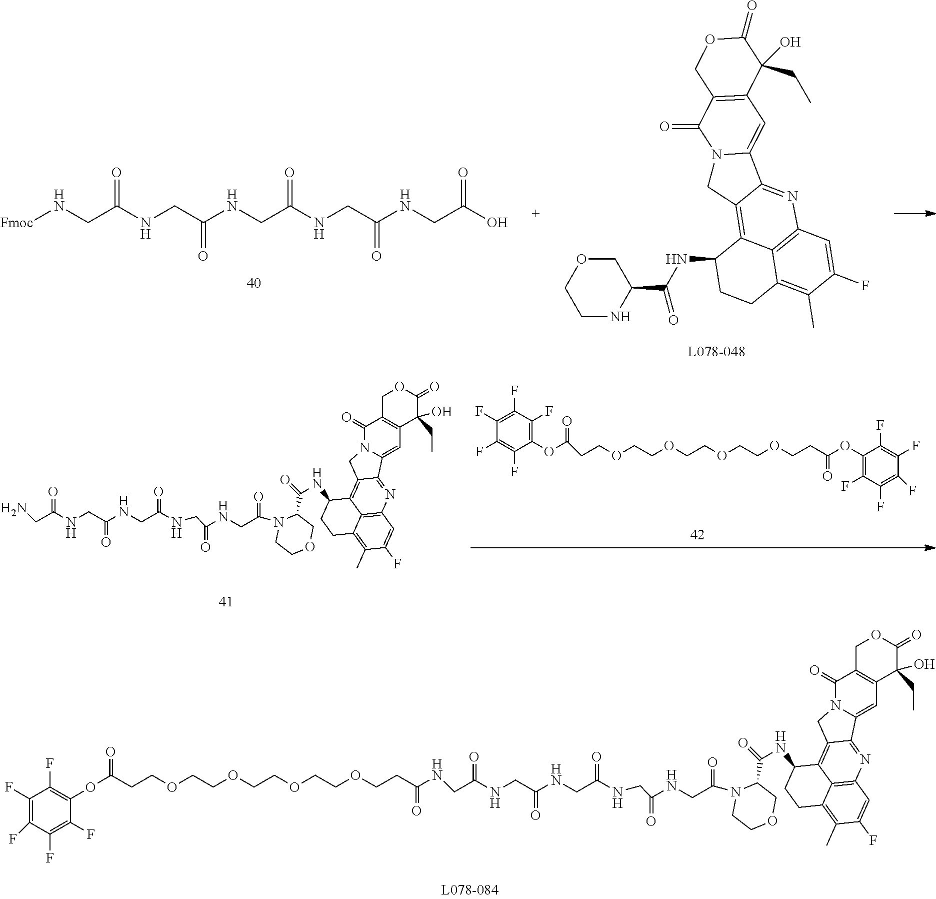

To a solution of compounds 40 (CAS #132742-00-8; 20 mg, 38 μmol), L078-048 (TFA salt, 25 mg, 38 μmol) and HATU (15 mg, 38 μmol) in 2 mL of DMF was added DIEA (10 mg, 76 μmol). The solution was stirred for 5 minutes. To the reaction mixture was added diethylamine (1 mL). The solution was stirred for additional 30 minutes. Diethylamine was removed by evaporation. The residue was purified by HPLC and dried to give compound 41 as a yellow powder.

Compound 41 was dissolved in 2 ml of DMF. To the solution of compound 41 was added compound 42 (20 mg, 32 μmol) and NaHCO₃ solution (0.5 mL, 5%) and stirred for 10 minutes. The solution was purified by HPLC to give compound L078-084 (3.2 mg) as a pale-yellow powder. MS m/z 1276.5 (M+H).

### Example S20: Synthesis of Compound L078-092

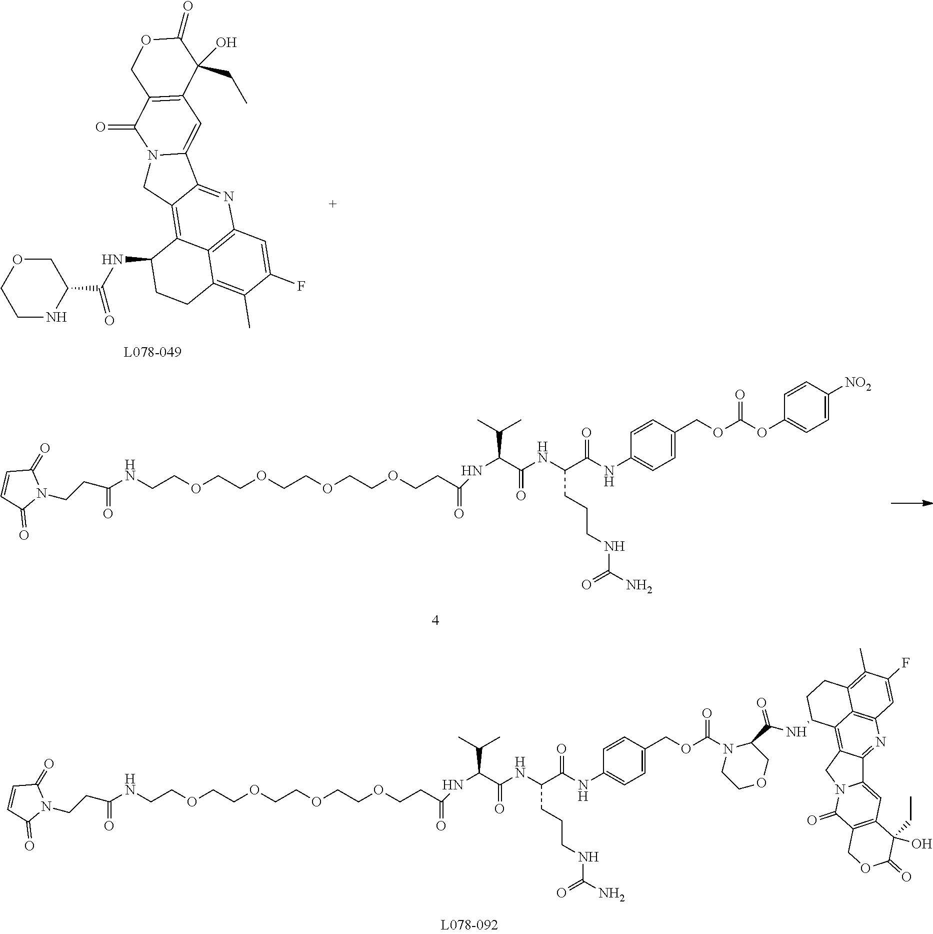 [SMILES: CC[C@]1(O)C(=O)OCc2c1cc1n(c2=O)Cc2c-1nc1cc(F)c(C)c3c1c2[C@H](NC(=O)[C@H]1COCCN1)CC3 ; CC(C)[C@H](NC(=O)CCOCCOCCOCCOCCNC(=O)CCN1C(=O)C=CC1=O)C(=O)N[C@@H](CCCNC(N)=O)C(=O)Nc1ccc(COC(=O)Oc2ccc([N+](=O)[O-])cc2)cc1 ; CC[C@]1(O)C(=O)OCc2c1cc1n(c2=O)Cc2c-1nc1cc(F)c(C)c3c1c2[C@H](NC(=O)[C@H]1COCCN1C(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1)CC3]

A solution of compounds L078-049 (TFA salt, 10 mg, 15 μmol), 4 (14 mg, 15 μmol), HOBt (1 mg, 7 μmol) and DIEA (4 mg, 30 μmol) in 2 mL of DMF was stirred for 2 hours. The residue was purified by HPLC to give compound L78-092 (10.1 mg) as a pale-yellow powder. MS m/z 1352.7 (M+H).

### Example S21: Synthesis of Compound L078-093

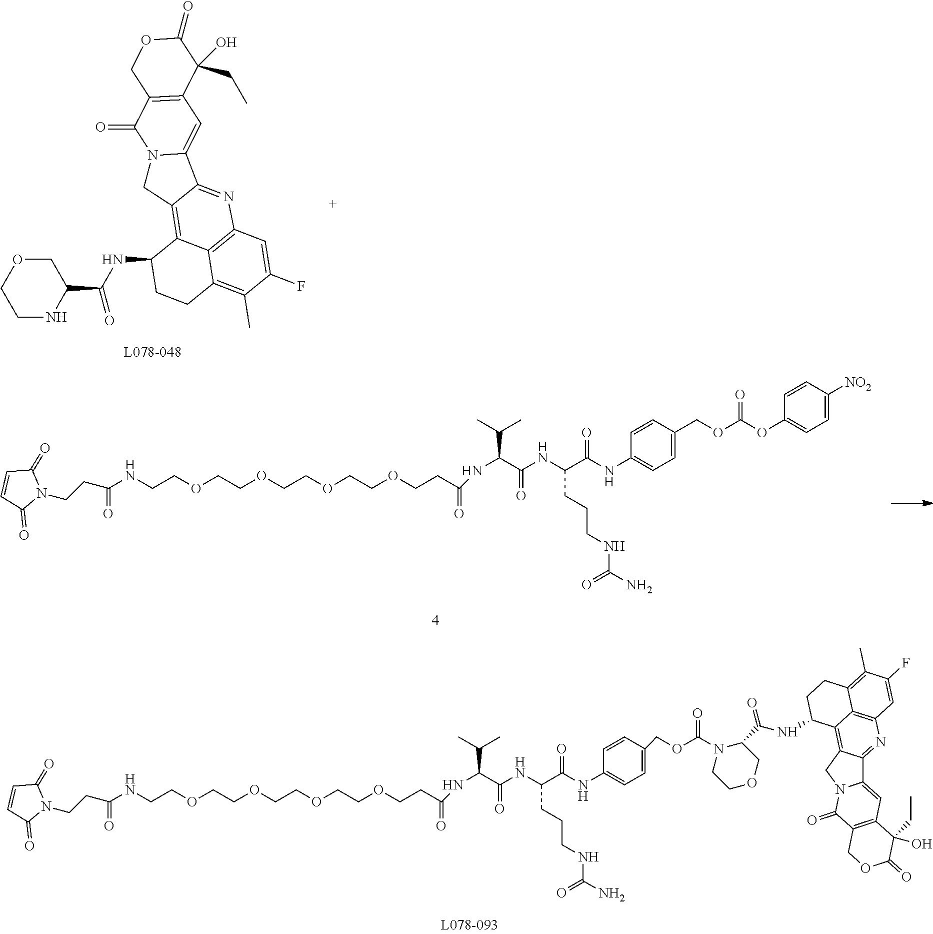

A solution of compounds L078-048 (TFA salt, 30 mg, 45 μmol), 4 (43 mg, 45 μmol), HOBt (4 mg, 30 μmol) and DIEA (12 mg, 90 μmol) in 2 mL of DMF was stirred for 2 hours. The residue was purified by HPLC to give compound L078-093 (51 mg) as a pale-yellow powder. MS m/z 1353.0 (M+H).

### Example S22: Synthesis of Compound L079-018

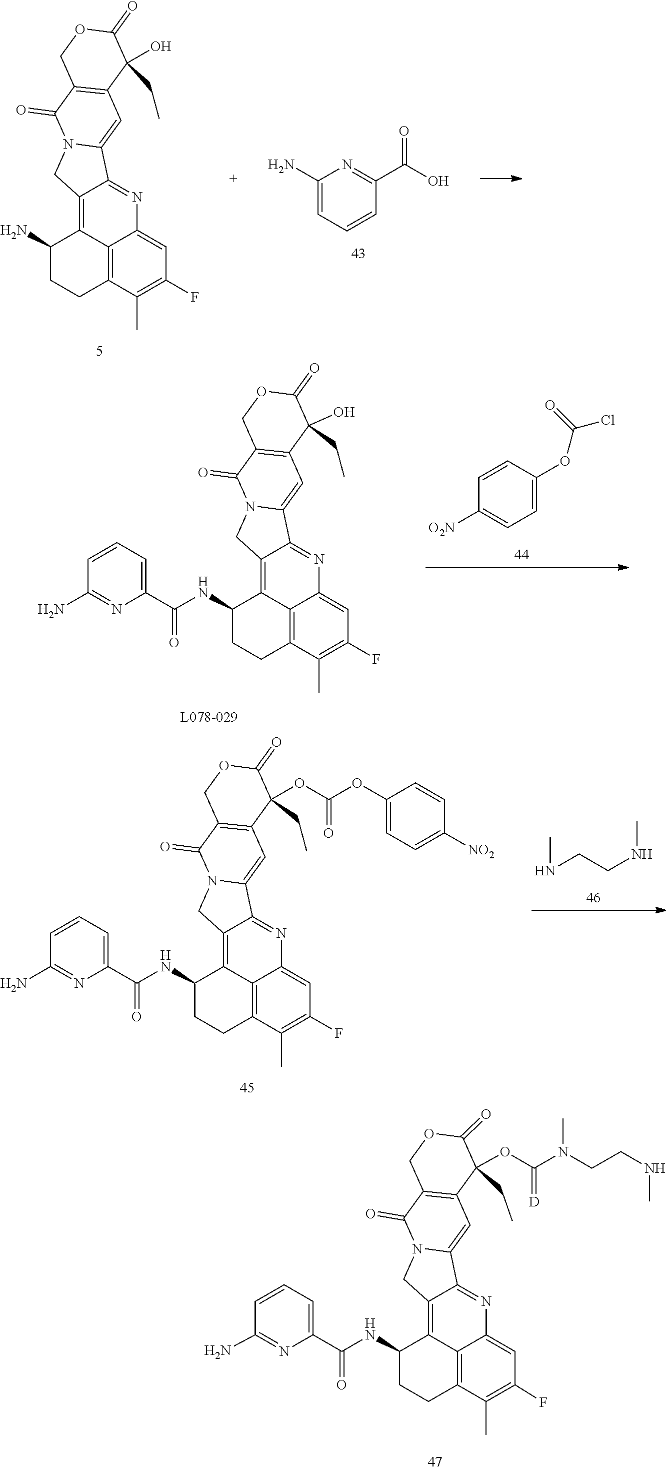

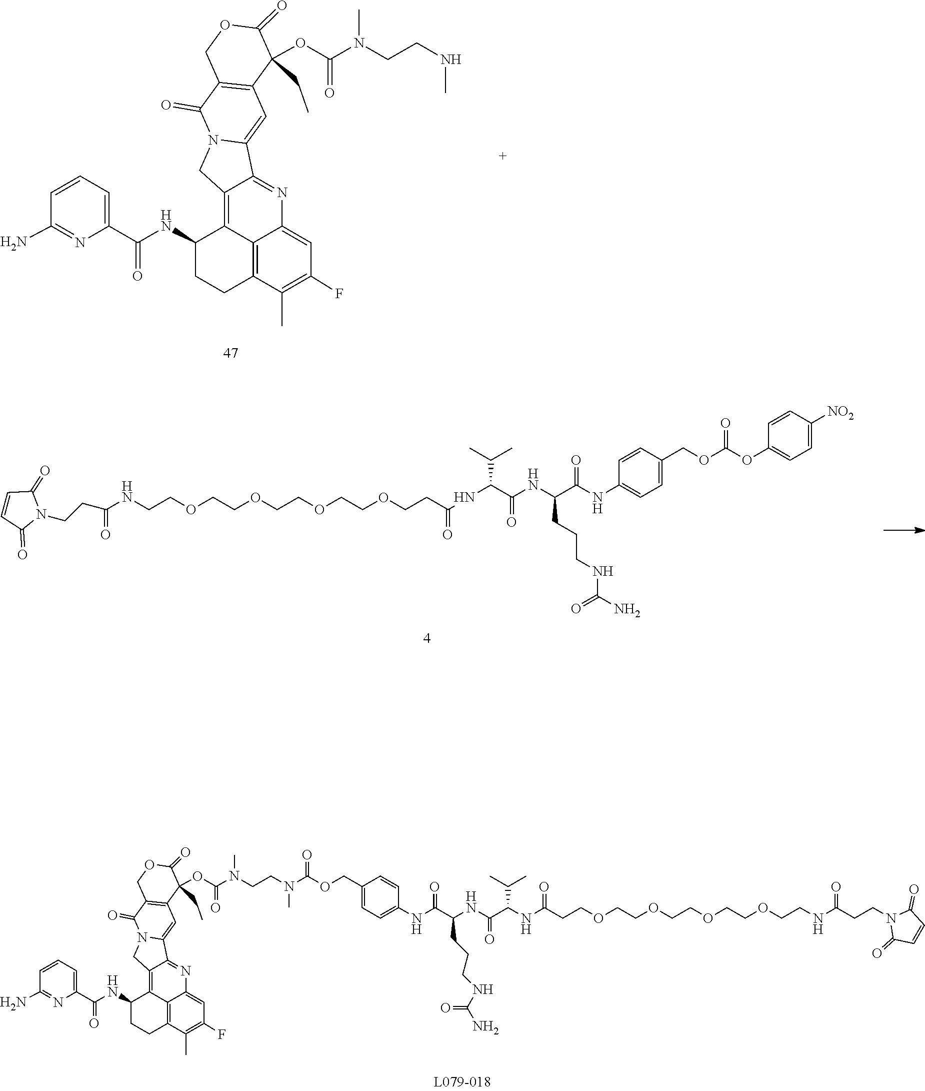 [SMILES: CC[C@]1(OC(=O)N(C)CCNC)C(=O)OCc2c1cc1n(c2=O)Cc2c-1nc1cc(F)c(C)c3c1c2[C@H](NC(=O)c1cccc(N)n1)CC3 ; CC(C)[C@@H](NC(=O)CCOCCOCCOCCOCCNC(=O)CCN1C(=O)C=CC1=O)C(=O)N[C@H](CCCNC(N)=O)C(=O)Nc1ccc(COC(=O)Oc2ccc([N+](=O)[O-])cc2)cc1 ; CC[C@]1(OC(=O)N(C)CCN(C)C(=O)OCc2ccc(NC(=O)[C@H](CCCNC(N)=O)NC(=O)[C@@H](NC(=O)CCOCCOCCOCCOCCNC(=O)CCN3C(=O)C=CC3=O)C(C)C)cc2)C(=O)OCc2c1cc1n(c2=O)Cc2c-1nc1cc(F)c(C)c3c1c2[C@H](NC(=O)c1cccc(N)n1)CC3]

DIEA (15 μL) was added slowly to a suspension of compounds 5 (mesylate salt, 10 mg, 0.0188 mmol), 43 (2.6 mg, 0.0188 mmol) and HATU (7.2 mg, 0.0188 mmol) in DMF (2 mL). The resulting mixture was stirred at RT for 15 minutes and purified by HPLC to give compound L078-029 (8 mg) as a yellowish powder. MS m/z 556.4 (M+H).

DMAP (60 mg, 0.5 mmol) was added to a suspension of compounds L078-029 (50 mg, 0.1 mmol) and 44 (60 mg, 0.3 mmol). The resulting mixture was stirred at r.t. for 3 hours and purified by HPLC to give compound 45 (10 mg) as a yellowish powder. MS m/z 721.1 (M+H).

Compound 46 (15 uL) was added to the solution of compound 45 (10 mg, 0.014 mmol) in DMF (2 mL). The resulting mixture was stirred at r.t. for 30 minutes and purified by HPLC to give compound 47 (13 mg) as a yellowish powder. MS m/z 670.3 (M+H).

DIEA (10 uL) was added slowly to the solution of compounds 47 (12 mg, 0.014 mmol), 4 (17 mg, 0.018 mmol) and HOAt (3 mg, 0.018 mmol). The resulting mixture was stirred at r.t. for 15 minutes and purified by HPLC to give compound L079-018 (8 mg) as a yellowish powder. MS m/z 1473.7 (M+H).

### Example S23: Synthesis of Compound L079-019

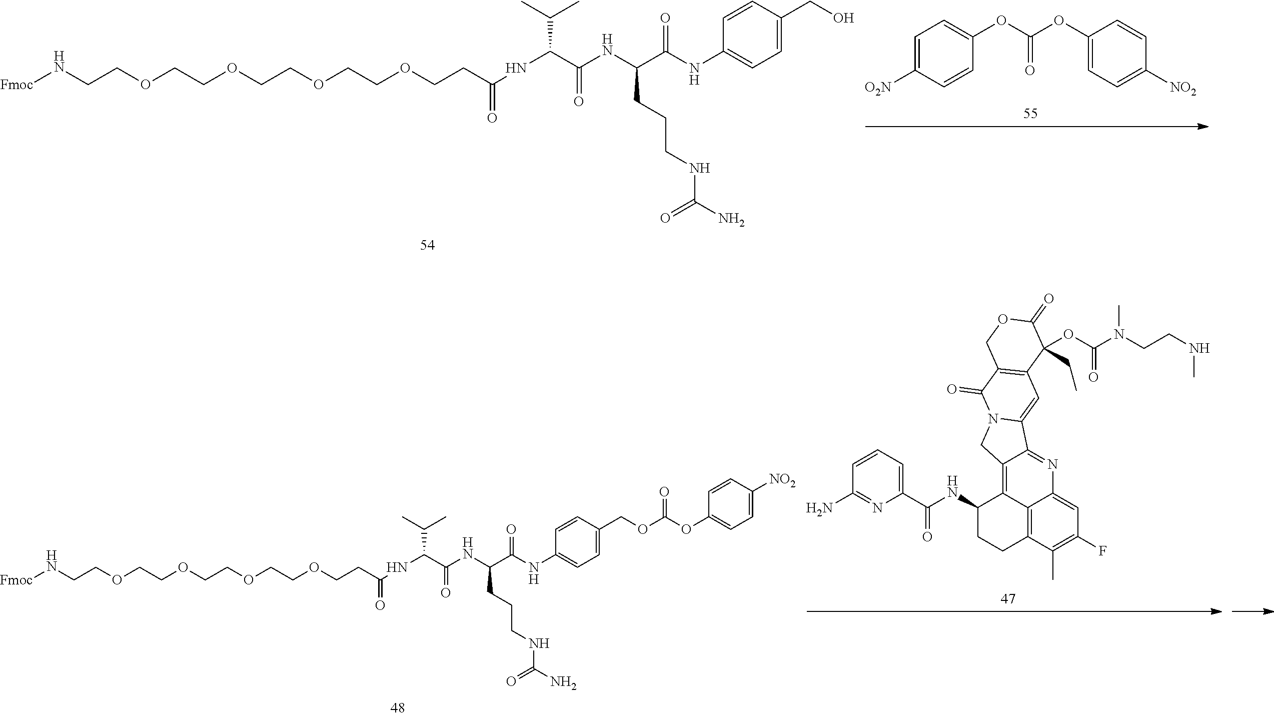

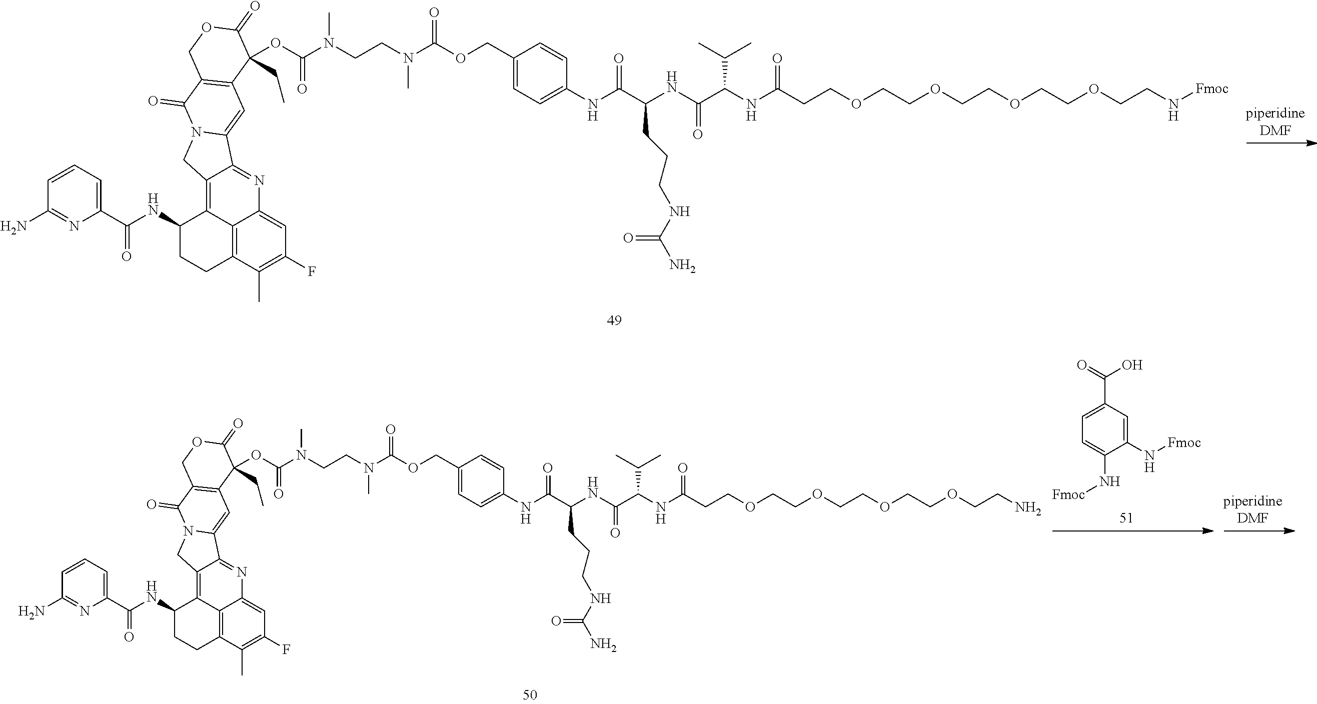

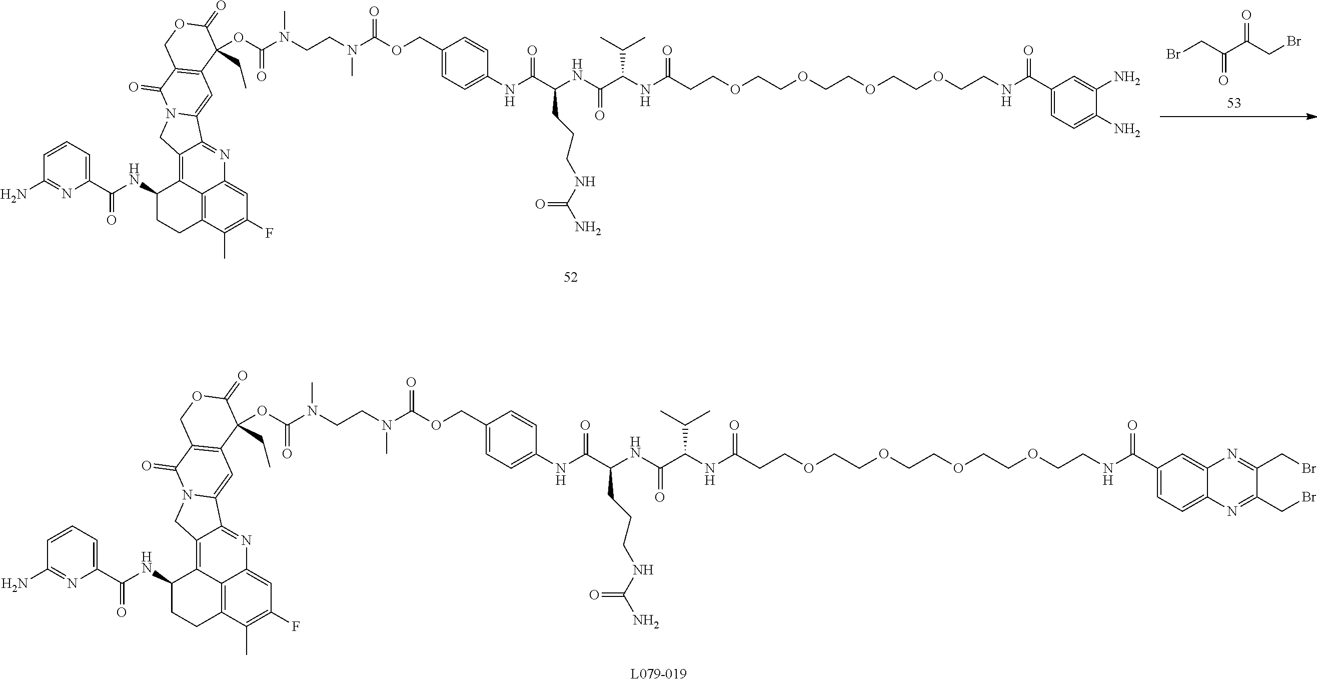 [SMILES: CC[C@]1(OC(=O)N(C)CCN(C)C(=O)OCc2ccc(NC(=O)[C@H](CCCNC(N)=O)NC(=O)[C@@H](NC(=O)CCOCCOCCOCCOCCNC(=O)c3ccc(N)c(N)c3)C(C)C)cc2)C(=O)OCc2c1cc1n(c2=O)Cc2c-1nc1cc(F)c(C)c3c1c2[C@H](NC(=O)c1cccc(N)n1)CC3 ; O=C(CBr)C(=O)CBr ; CC[C@]1(OC(=O)N(C)CCN(C)C(=O)OCc2ccc(NC(=O)[C@H](CCCNC(N)=O)NC(=O)[C@@H](NC(=O)CCOCCOCCOCCOCCNC(=O)c3ccc4nc(CBr)c(CBr)nc4c3)C(C)C)cc2)C(=O)OCc2c1cc1n(c2=O)Cc2c-1nc1cc(F)c(C)c3c1c2[C@H](NC(=O)c1cccc(N)n1)CC3]

A solution of compound 54 (Broadpharm; 300 mg, 0.354 mmol), compound 55, (161 mg, 0.531 mmol) and DIEA (68 mg, 0.531 mmol) in DMF (5 mL) was stirred for 5 hours. The crude was purified by HPLC to give compound 48 as an off-white powder.

DIEA (8 μL) was added slowly to a solution of compound 47 (7 mg, 0.0105 mmol), 48 (12 mg, 0.0118 mmol) and HOAt (1.4 mg, 0.0103 mmol) in DMF (2 ml). The resulting mixture was stirred at r.t. for 30 minutes and purified by HPLC to give compound 49 (15 mg) as a yellowish powder. MS m/z 1545.9 (M+H).

Piperidine (100 μL) was added slowly to the solution of compound 49 (15 mg, 0.0097 mmol) in DMF (2 mL). The resulting mixture was stirred at r.t. for 15 minutes and purified by HPLC to give compound 50 (10 mg) as a yellowish powder.

DIEA (6 μL) was added slowly to a solution of compounds 50 (10 mg, 0.0076 mmol), 51 (5 mg, 0.0084 mmol) and HATU (3 mg, 0.0079 mmol) in DMF (2 mL). The resulting mixture was stirred at RT for 30 minutes followed by the addition of piperidine (100 μL) and stirred for another 15 minutes. The resulting mixture was purified by HPLC to give compound 52 (15 mg) as a yellowish powder.

The mixture of compound 52 (15 mg, 0.0103 mmol) and 53 (8 mg, 0.0328 mmol) in acetonitrile/water was stirred at RT for 15 minutes and purified by HPLC to give compound L079-019 (3 mg) as a yellowish powder. MS m/z 1663.5 (M+H).

### Example S24: Synthesis of Compound L079-027

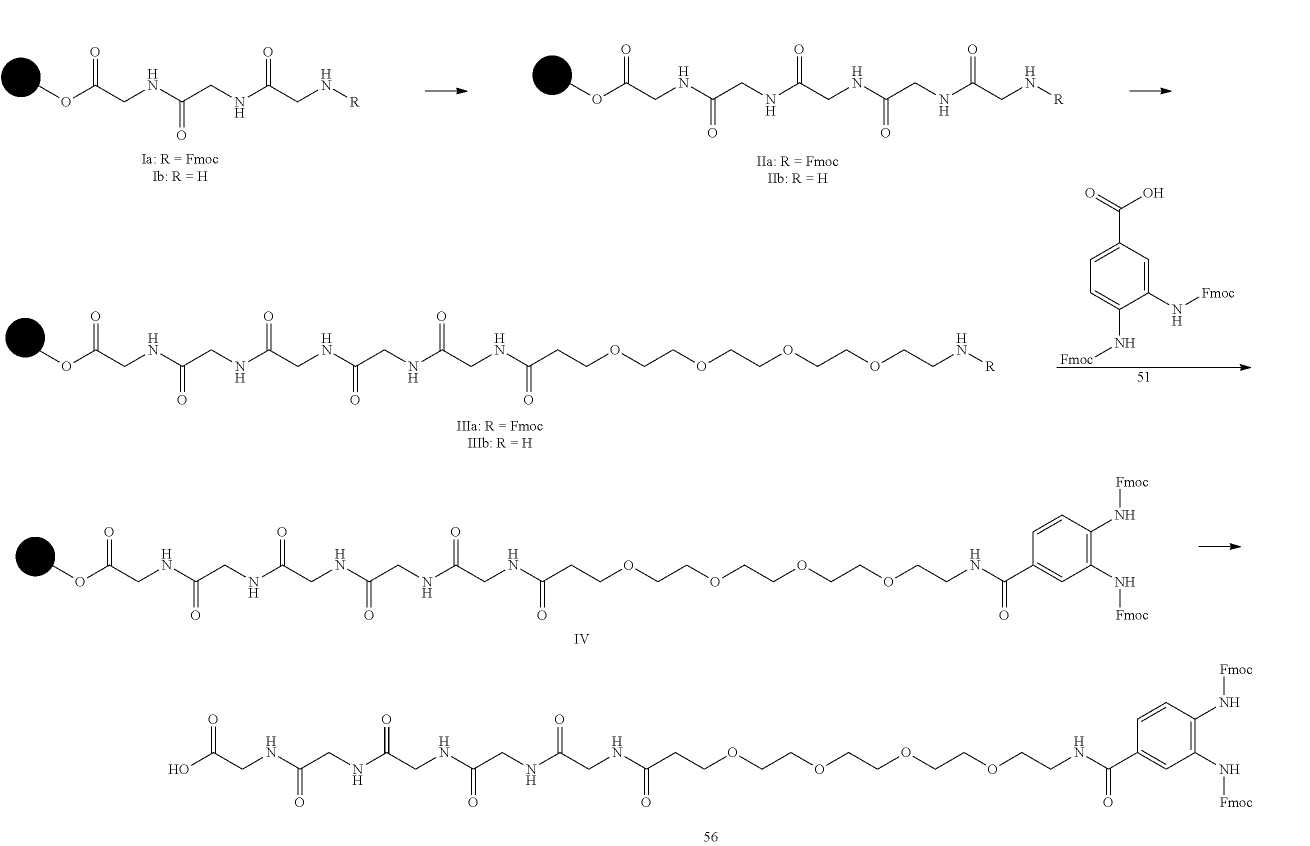

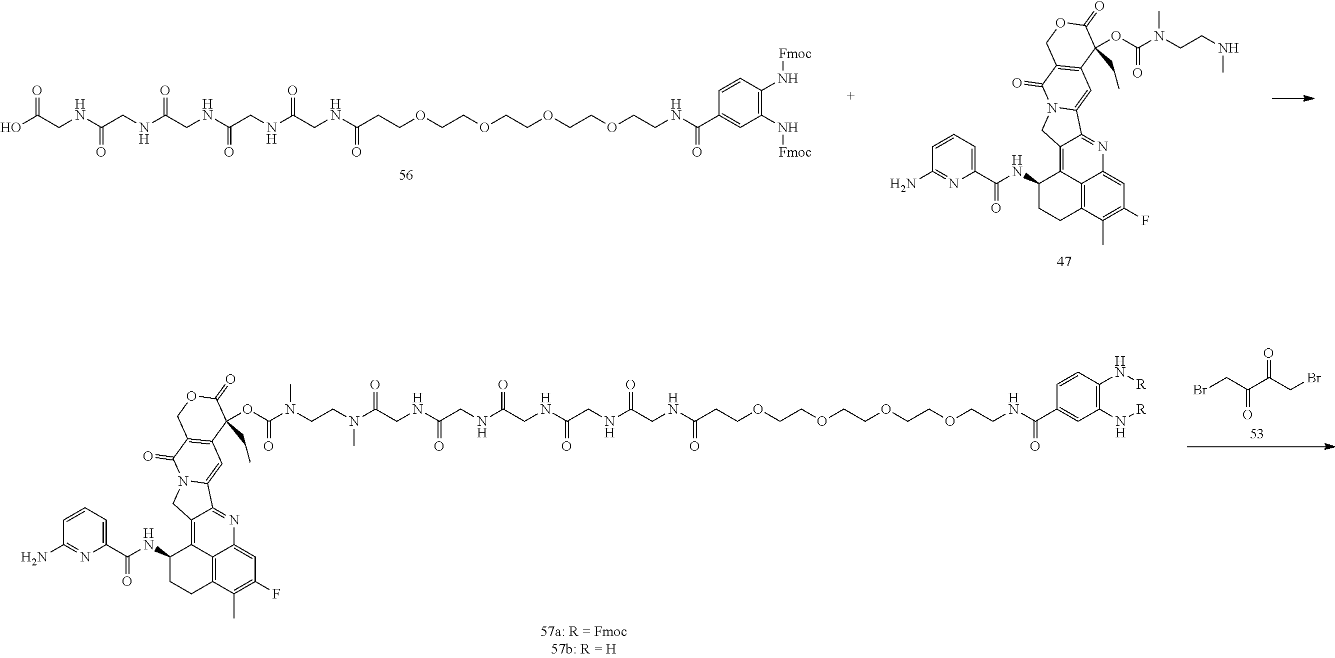

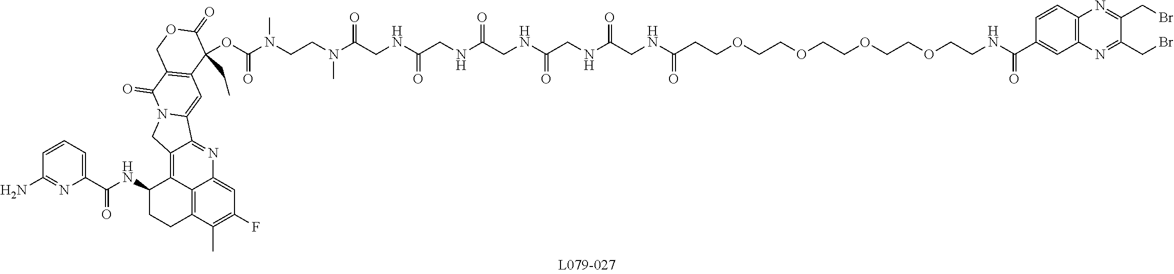 [SMILES: CC[C@]1(OC(=O)N(C)CCN(C)C(=O)CNC(=O)CNC(=O)CNC(=O)CNC(=O)CNC(=O)CCOCCOCCOCCOCCNC(=O)c2ccc3nc(CBr)c(CBr)nc3c2)C(=O)OCc2c1cc1n(c2=O)Cc2c-1nc1cc(F)c(C)c3c1c2[C@H](NC(=O)c1cccc(N)n1)CC3]

DIEA (12 mL) was added slowly to the suspension of Fmoc-Gly-Gly-Gly-OH (6.7 g, 16.2 mmol) in dichloromethane (200 mL). The resulting mixture was added to 500 mL reaction vessel filled with 2-Chlorotrityl chloride resin (20 g, 16.2 mmol). After shaking at room temperature for 1 hour, the resin was filtered and washed with DMF (300 mL×3) to give resin Ia. Then the resin was treated with 25% Piperidine in DMF (200 mL) at room temperature for 30 minutes. The resin was filtered and washed with DMF (300 mL×3) to give resin Ib (23.5 g, 16.2 mmol).

Fmoc-Gly-Gly-OH (2.13 g, 6.2 mmol) and Oxyma Pure (CAS 3849-21-6; 0.86 g, 6 mmol) was dissolved in anhydrous DMF (100 mL). The resulting mixture was added slowly to 250 mL reaction vessel filled with resin Ib (7.5 g, 5 mmol) followed by addition of N,N′-diisopropylcarbodiimide (4 mL, 25.5 mmol). The vessel was shaken at room temperature for 2 hours. The resin was filtered and washed with DMF (100 mL×3) to give resin Iia. Then the resin was treated with 25% Piperidine in DMF (100 mL) at room temperature for 30 minutes. The resin was filtered and washed with DMF (100 mL×3) to give resin Jib.

DIEA (2.1 mL) was added slowly to the mixture of Fmoc-NH-PEG4-CH₂CH₂COOH (2.93 g, 6 mmol) and PyAOP (3.1 g, 6 mmol) in DMF (100 mL). The resulting mixture was slowly added to resin Jib in the vessel. After shaking at room temperature for 1 hour, the resin was filtered and washed with DMF (100 mL×3) to give resin IIIa. Then the resin was treated with 25% Piperidine in DMF (100 mL) at room temperature for 30 minutes. The resin was filtered and washed with DMF (100 mL×3) to give resin IIIb.

DIEA (2.1 mL) was added slowly to the mixture of compound 51 (3.6 g, 6 mmol) and PyAOP (3.1 g, 6 mmol) in DMF (100 mL). The resulting mixture was slowly added to resin IIIb in the vessel. After shaking at room temperature for 1 hour, the resin was filtered and washed with DMF (100 mL×3) and dichloromethane (100 mL×2) to give resin IV. Then the resin was treated with 5% TFA in dichloromethane (100 mL) to give compound 56 (0.95 g).

DIEA (4 μL) was added slowly to the solution of compound 47 (4 mg, 0.00605 mmol), compound 56 (7 mg, 0.00628 mmol) and HATU (2.4 mg, 0.0060 mmol) in DMF (2 mL). The resulting mixture was stirred at RT for 30 minutes followed by addition of piperidine (100 μL) for another 15 minutes. The mixture was purified by HPLC to give compound 57b (5 mg) as a yellowish powder.

The mixture of compound 57b (5 mg, 0.0037 mmol) and compound 53 (3 mg, 0.0123 mmol) in acetonitrile-H₂O was stirred at RT for 15 minutes and purified by HPLC to give compound L079-027 (2 mg) as a yellowish powder. MS m/z 1544.8 (M+H).

### Example S25: Synthesis of Compound L079-034

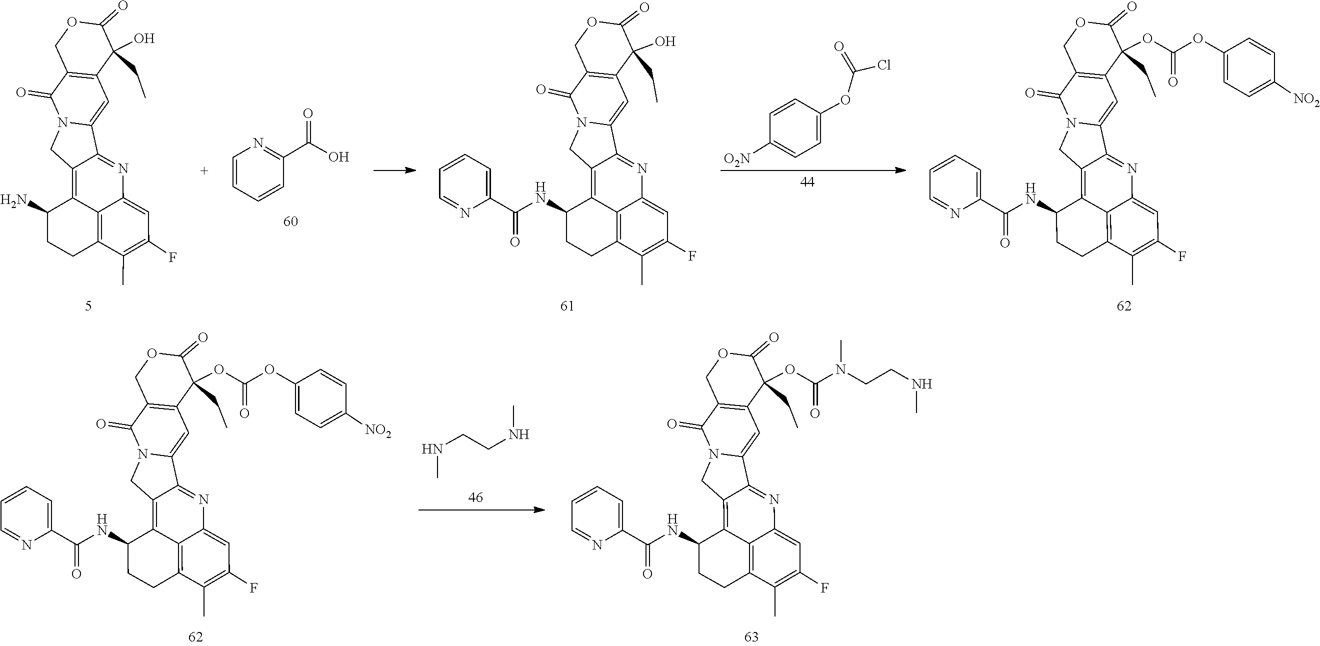

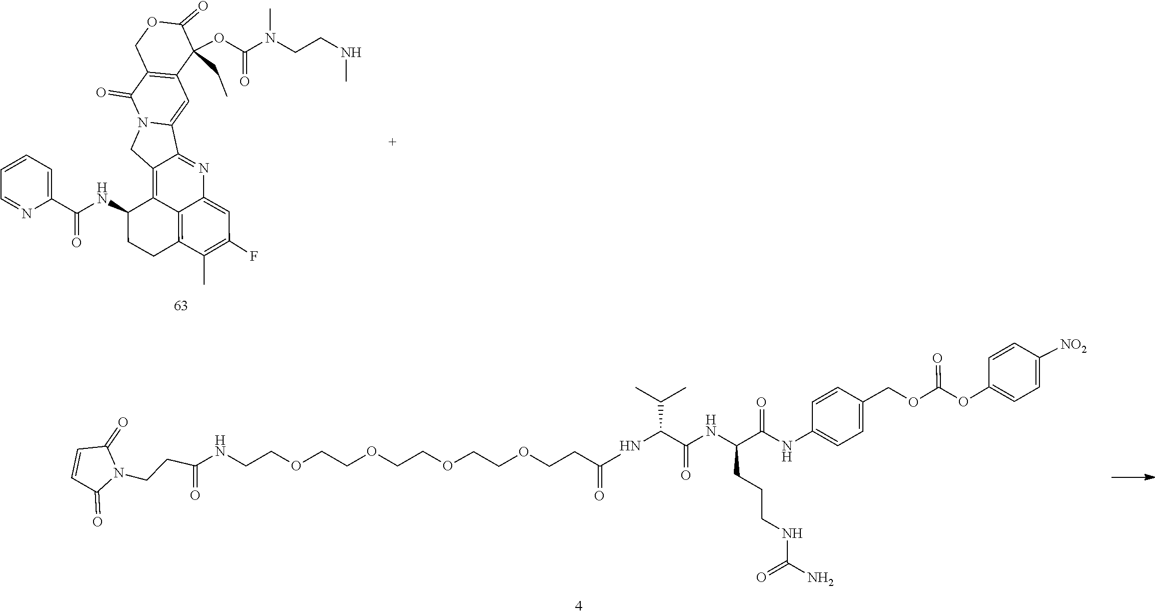 [SMILES: CC[C@]1(OC(=O)N(C)CCNC)C(=O)OCc2c1cc1n(c2=O)Cc2c-1nc1cc(F)c(C)c3c1c2[C@H](NC(=O)c1ccccn1)CC3 ; CC(C)[C@@H](NC(=O)CCOCCOCCOCCOCCNC(=O)CCN1C(=O)C=CC1=O)C(=O)N[C@H](CCCNC(N)=O)C(=O)Nc1ccc(COC(=O)Oc2ccc([N+](=O)[O-])cc2)cc1]

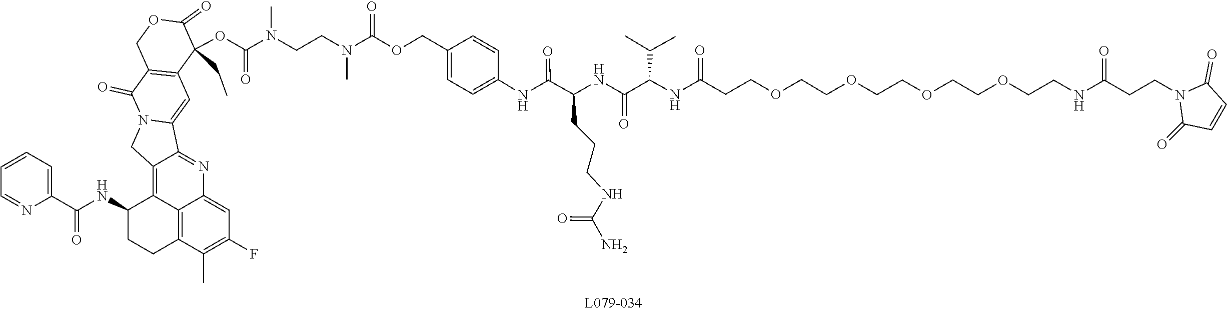 [SMILES: CC[C@]1(OC(=O)N(C)CCN(C)C(=O)OCc2ccc(NC(=O)[C@H](CCCNC(N)=O)NC(=O)[C@@H](NC(=O)CCOCCOCCOCCOCCNC(=O)CCN3C(=O)C=CC3=O)C(C)C)cc2)C(=O)OCc2c1cc1n(c2=O)Cc2c-1nc1cc(F)c(C)c3c1c2[C@H](NC(=O)c1ccccn1)CC3]

DIEA (60 μL) was added slowly to the suspension of compound 5 (mesylate salt, 33 mg, 0.0940 mmol), compound 60 (12 mg, 0.0975 mmol) and HATU (36 mg, 0.0947 mmol) in DMF (2 mL). The resulting mixture was stirred at RT for 15 minutes and purified by HPLC to give compound 6 (33 mg) as a yellowish powder. MS m/z 541.5 (M+H).

DMAP (45 mg, 0.3683 mmol) was added to a suspension of compound 61 (33 mg, 0.061 mmol) and compound 44 (61 mg, 0.3026 mmol) in Dichloromethane. The resulting mixture was stirred at RT for 3 hours and purified by HPLC to give compound 62 (40 mg) as a yellowish powder. MS m/z 706.3 (M+H).

Compound 46 (15 μL) was added to the solution of compound 62 (10 mg, 0.014 mmol) in DMF (2 mL). The resulting mixture was stirred at RT for 30 minutes and purified by HPLC to give compound 63 (11 mg) as a yellowish powder. MS m/z 655.4 (M+H).

DIEA (10 μL) was added slowly to the solution of compound 63 (10 mg, 0.015 mmol), compound 4 (15 mg, 0.0159 mmol) and HOAt (2 mg, 0.0147 mmol) in DMF (2 mL). The resulting mixture was stirred at RT for 30 minutes and purified by HPLC to give compound L079-034 (9 mg) as a yellowish powder. MS m/z 1459.2 (M+H).

### Example S26: Synthesis of Compound L079-035

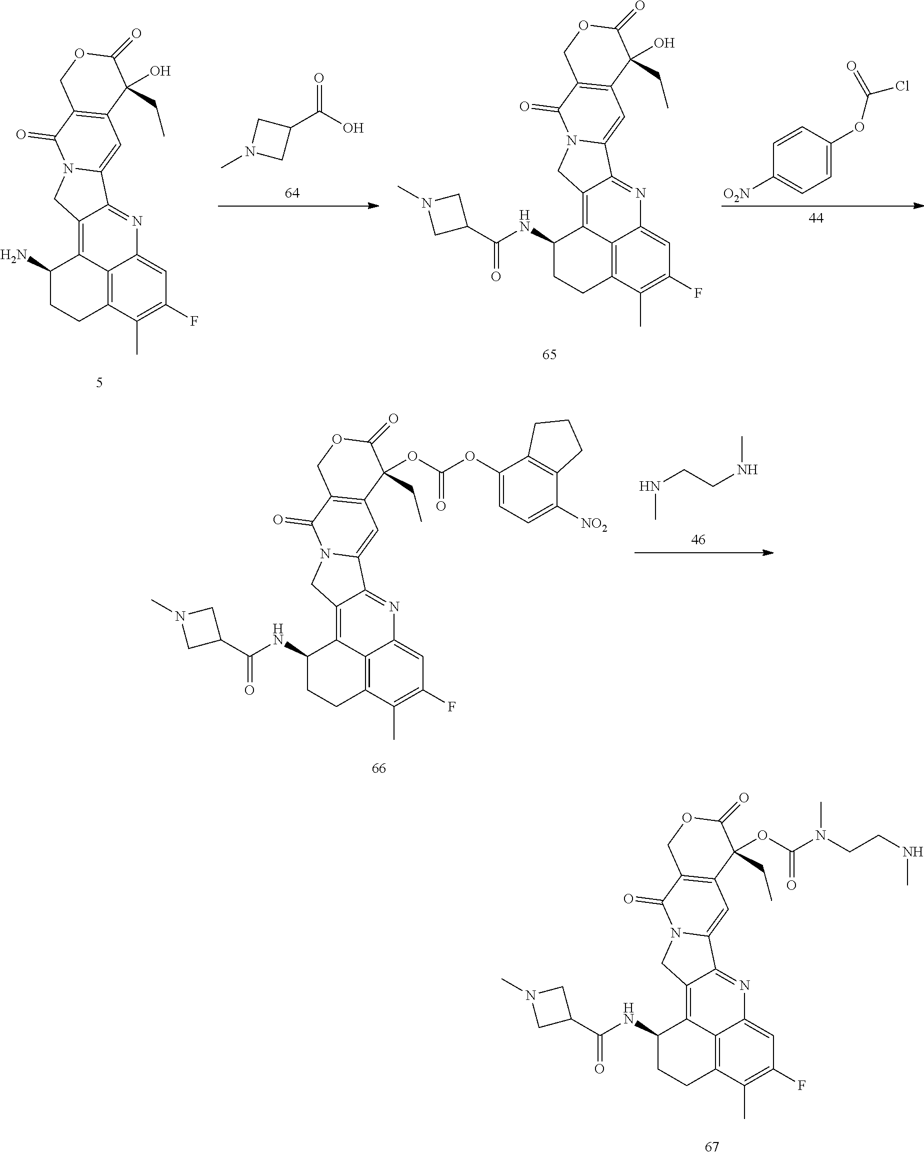

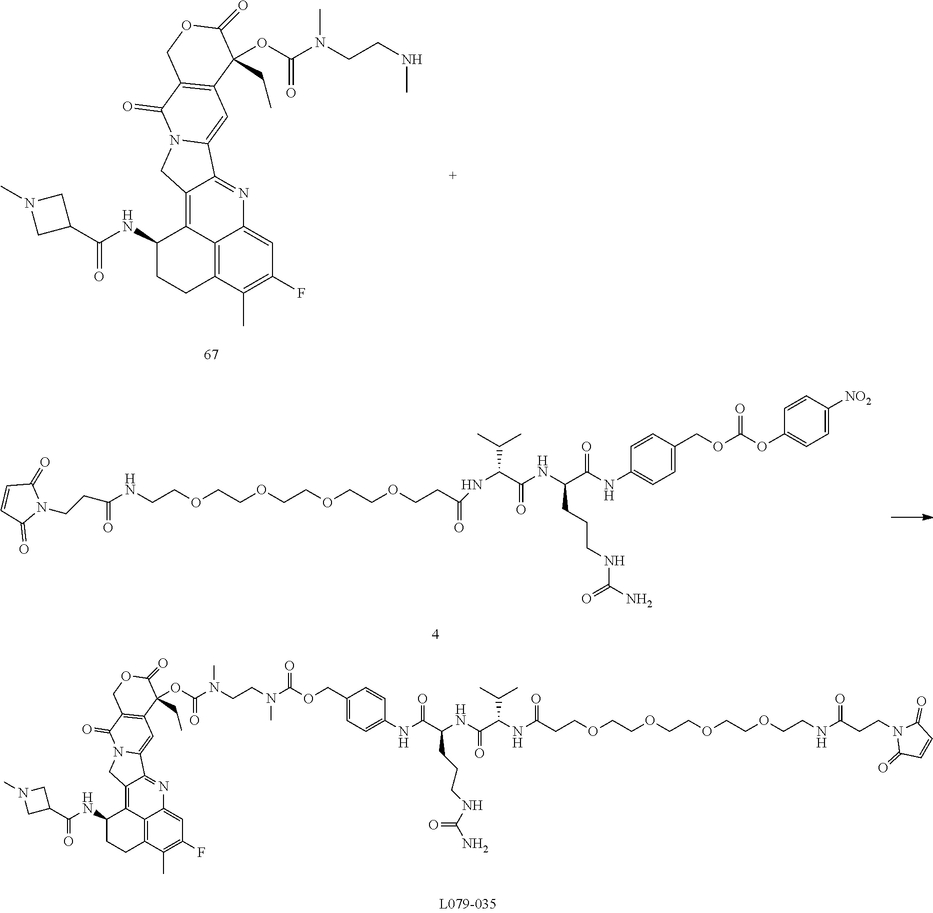

DIEA (80 μL) was added slowly to the suspension of compound 5_(mesylate salt, 53 mg, 0.1 mmol), compound 64 (11.5 mg, 0.1 mmol) and HATU (38 mg, 0.1 mmol) in DMF (2 mL). The resulting mixture was stirred at RT for 15 minutes and purified by HPLC to give compound 65 (62 mg) as a yellowish powder.

DMAP (41 mg, 0.3355 mmol) was added to the suspension of compound 65 (30 mg, 0.056 mmol) and compound 44 (57 mg, 0.2835 mmol) in dichloromethane. The resulting mixture was stirred at RT for 3 hours and purified by HPLC to give compound 66 (10 mg) as a yellowish powder.

Compound 46 (12 μL) was added to the solution of compound 66 (10 mg, 0.0143 mmol) in DMF (2 mL). The resulting mixture was stirred at RT for 30 minutes and purified by HPLC to give compound 67 (6 mg) as a yellowish powder.

DIEA (5 μL) was added slowly to the solution of compound 67 (6 mg, 0.0092 mmol), compound 4 (8 mg, 0.0085 mmol) and HOAt (1.3 mg, 0.0095 mmol) in DMF (2 mL). The resulting mixture was stirred at RT for 30 minutes and purified by HPLC to give compound L079-035 (2.4 mg) as a yellowish powder. MS m/z 1451 (M+H).

### Example S27: Synthesis of Compound L079-040

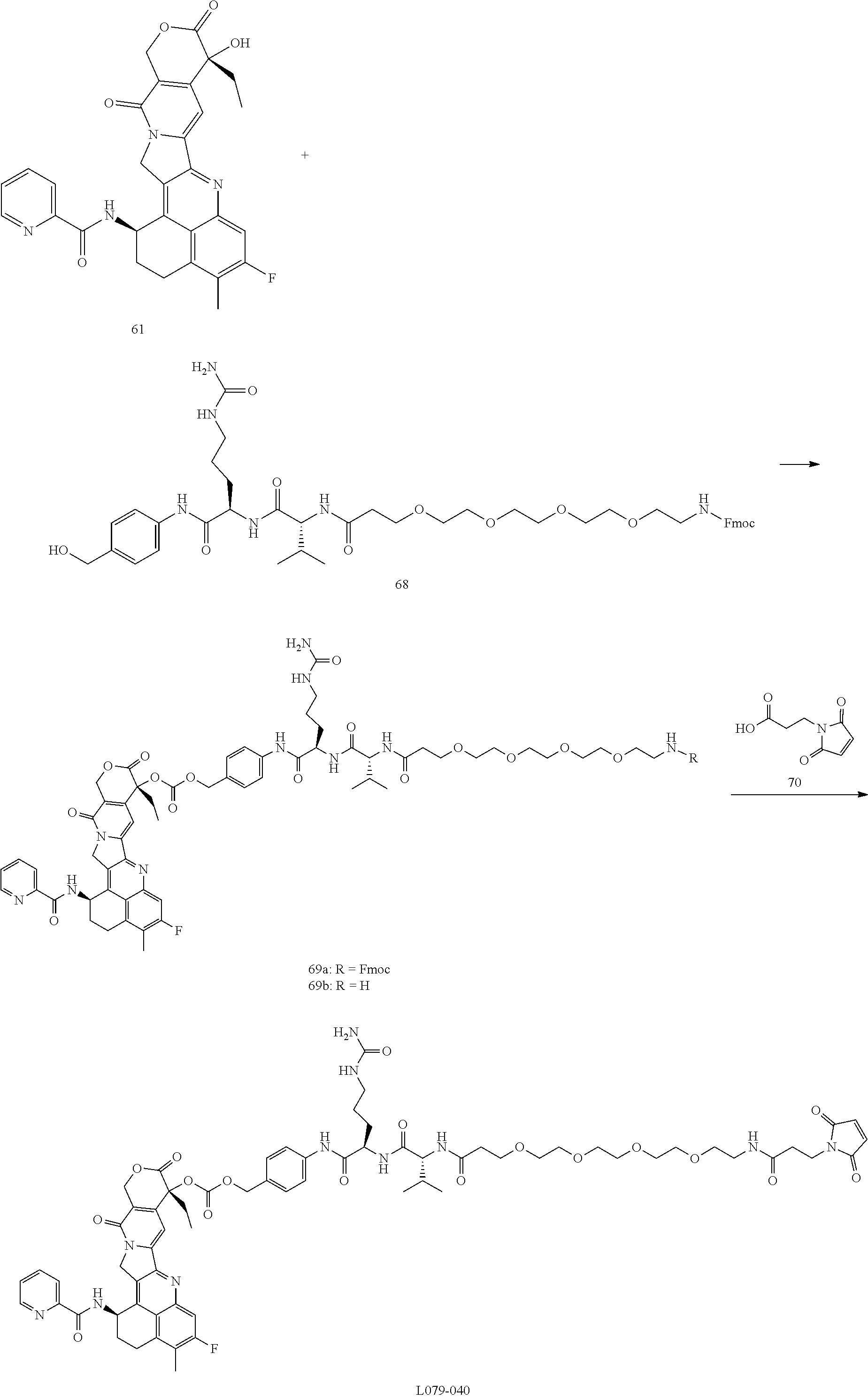

The mixture of compound 61 (20 mg, 0.037 mmol), triphosgene (8.5 mg, 0.0287 mmol) and DMAP (23 mg, 0.1885 mmol) in dichloromethane (2 mL) was stirred at RT for 30 minutes followed by addition of compound 68 (CAS 2055024-58-1; 47 mg, 0.055 mmol) in DMF (0.5 mL) and DIEA (10 μL). The mixture was stirred for another 30 minutes. The mixture was purified by HPLC to give compound 69a (10 mg) as a yellowish powder.

Piperidine (100 μL) was added slowly to the solution of compound 69a (10 mg, 0.007 mmol) in DMF (2 mL). The resulting mixture was stirred at RT for 15 minutes and purified by HPLC to give compound 69b (8 mg) as a yellowish powder.

DIEA (8 μL) was added slowly to the solution of compound 69b (8 mg, 0.0067 mmol), compound 70 (2 mg, 0.0118 mmol) and HATU (5 mg, 0.0131 mmol) in DMF (2 mL). The resulting mixture was stirred at RT for 30 minutes and purified by HPLC to give compound L079-040 (3.7 mg) as a yellowish powder. MS m/z 1345 (M+H).

### Example S28: Synthesis of Compound L078-121

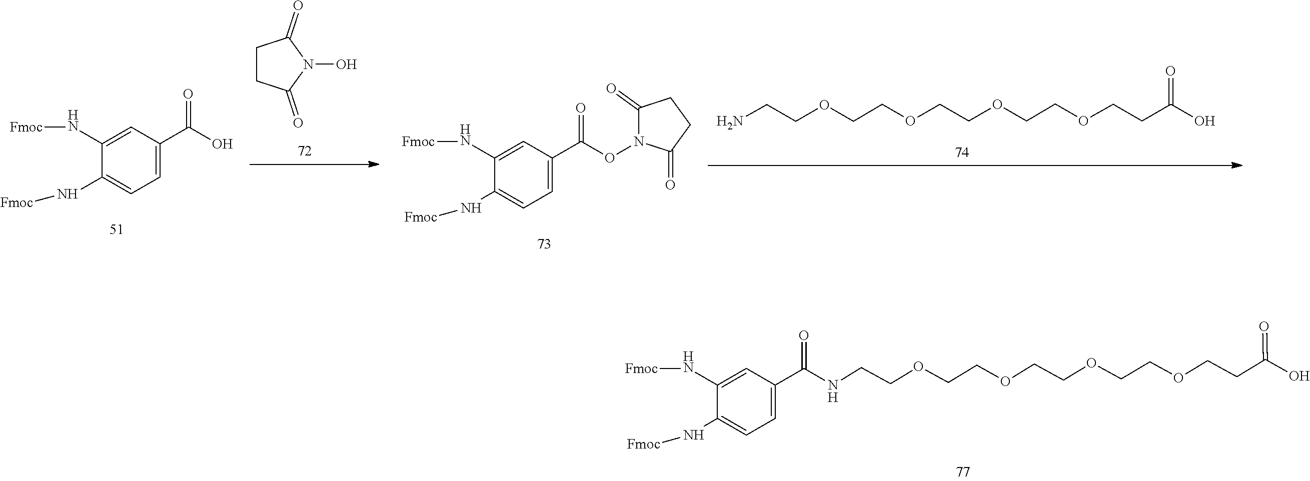

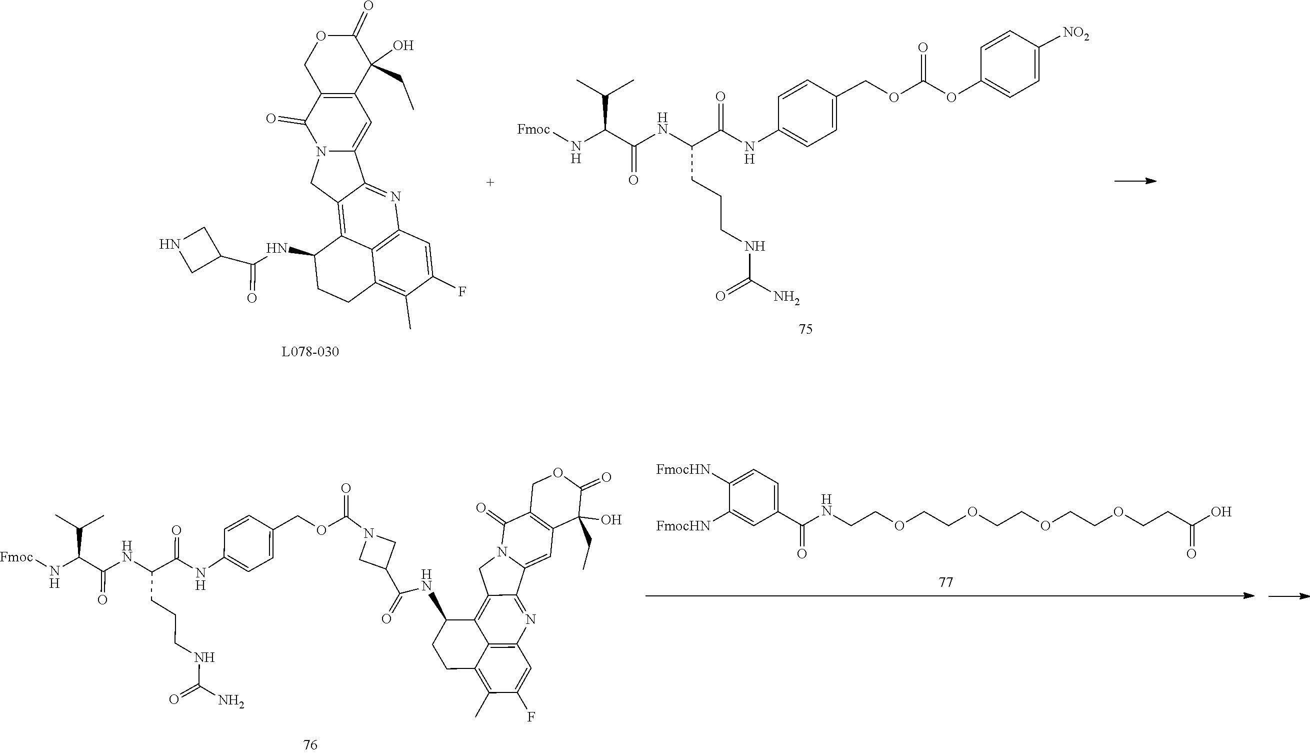

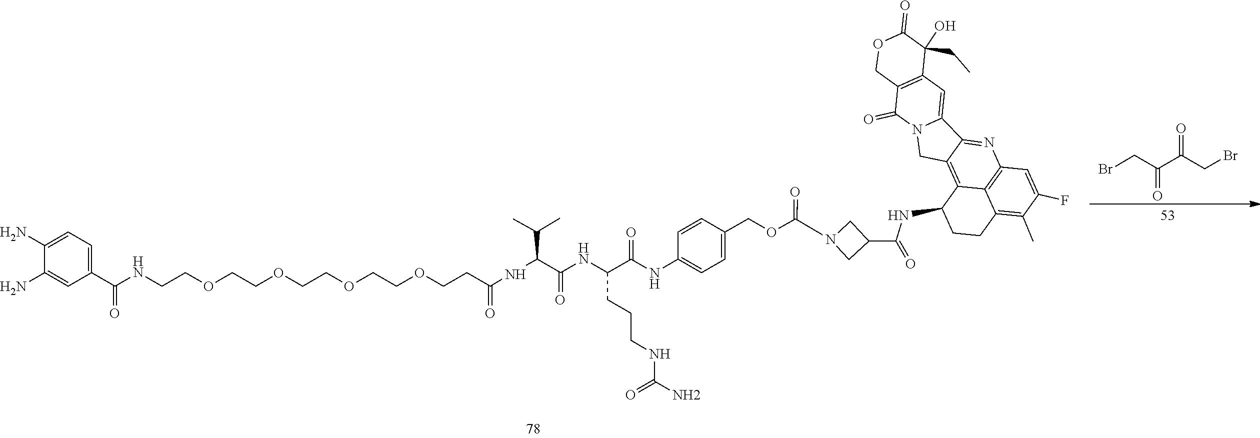 [SMILES: CC[C@]1(O)C(=O)OCc2c1cc1n(c2=O)Cc2c-1nc1cc(F)c(C)c3c1c2[C@H](NC(=O)C1CN(C(=O)OCc2ccc(NC(=O)[C@H](CCCNC(N)=O)NC(=O)[C@@H](NC(=O)CCOCCOCCOCCOCCNC(=O)c4ccc(N)c(N)c4)C(C)C)cc2)C1)CC3 ; O=C(CBr)C(=O)CBr]

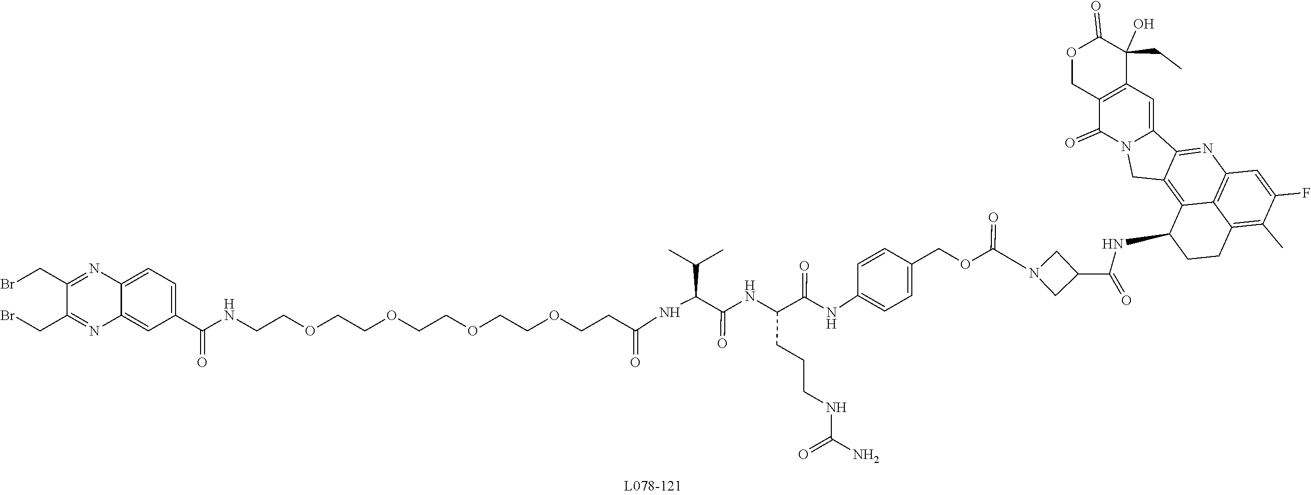 [SMILES: CC[C@]1(O)C(=O)OCc2c1cc1n(c2=O)Cc2c-1nc1cc(F)c(C)c3c1c2[C@H](NC(=O)C1CN(C(=O)OCc2ccc(NC(=O)[C@H](CCCNC(N)=O)NC(=O)[C@@H](NC(=O)CCOCCOCCOCCOCCNC(=O)c4ccc5nc(CBr)c(CBr)nc5c4)C(C)C)cc2)C1)CC3]

Compound 72 (386 mg, 3.356 mmol) was dissolved in DMF (5 mL). The solution was added into a solution of compound 51 (CAS #345958-22-7; 1 g, 1.678 mmol) and EDC-HCl (987 mg, 5.030 mmol) in DCM (50 mL). The resulting solution was stirred for 30 minutes. The crude was extracted with DCM, washed with water, and concentrated to give compound 73. Crude compound 73 was dissolved in 50 mL of CH₃CN/H₂O followed by addition of 5% NaHCO₃ solution (to adjust pH of solution to pH 8). Compound 74 (277 mg, 1.678 mmol) was added to the solution of compound 73, and the resulting solution was stirred for 30 minutes. It was then concentrated, and purified by HPLC to give compound 77 (1.12 g) MS m/z 844.2 (M+H).

A solution of compound L078-030 (40 mg, 0.063 mmol), compound 75 (CAS 863971-53-3; 73 mg, 0.093 mmol), HOBT (5 mg) and DIEA (16 mg, 0.124 mmol) in DMF (3 mL) was stirred for 2 hours. To the solution was added 0.5 ml of diethylamine and stirred for another 30 minutes. The mixture was purified by HPLC to give compound 76 (61 mg).

To a solution of compound 76 (61 mg, 0.059 mmol), compound 77 (50 mg, 0.059 mmol) and HATU (23 mg, 0.059 mmol) in DMF (3 mL) was added DIEA (19 mg, 0.147 mmol). The solution was stirred for 5 minutes followed by addition of 0.5 mL of diethylamine. The mixture was stirred for another 30 minutes and purified by HPLC to give compound 78 (42 mg).

Compound 78 (42 mg) was dissolved in 3 ml of 60% CH₃CN/H₂O (1% TFA). To the solution was added a solution of compound 53 (10 mg) in acetonitrile, stirred for 5 minutes and purified by HPLC to give compound L078-121 (25 mg), MS m/z 1513.7 (M+H).

### Example S29: Synthesis of Compound L078-118

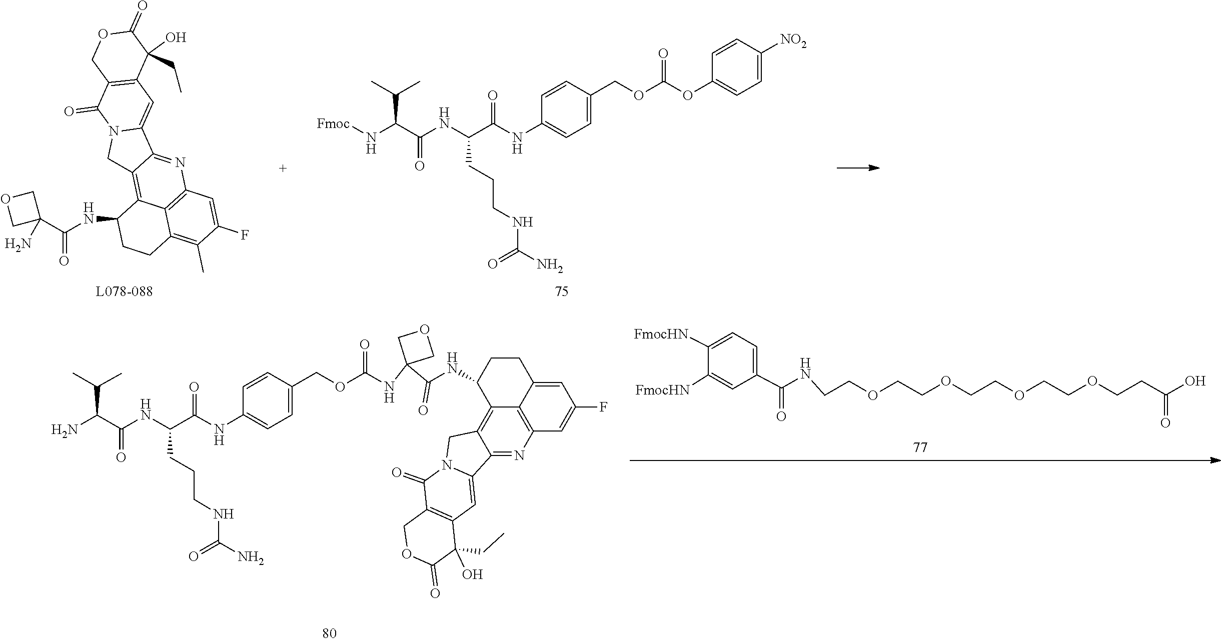

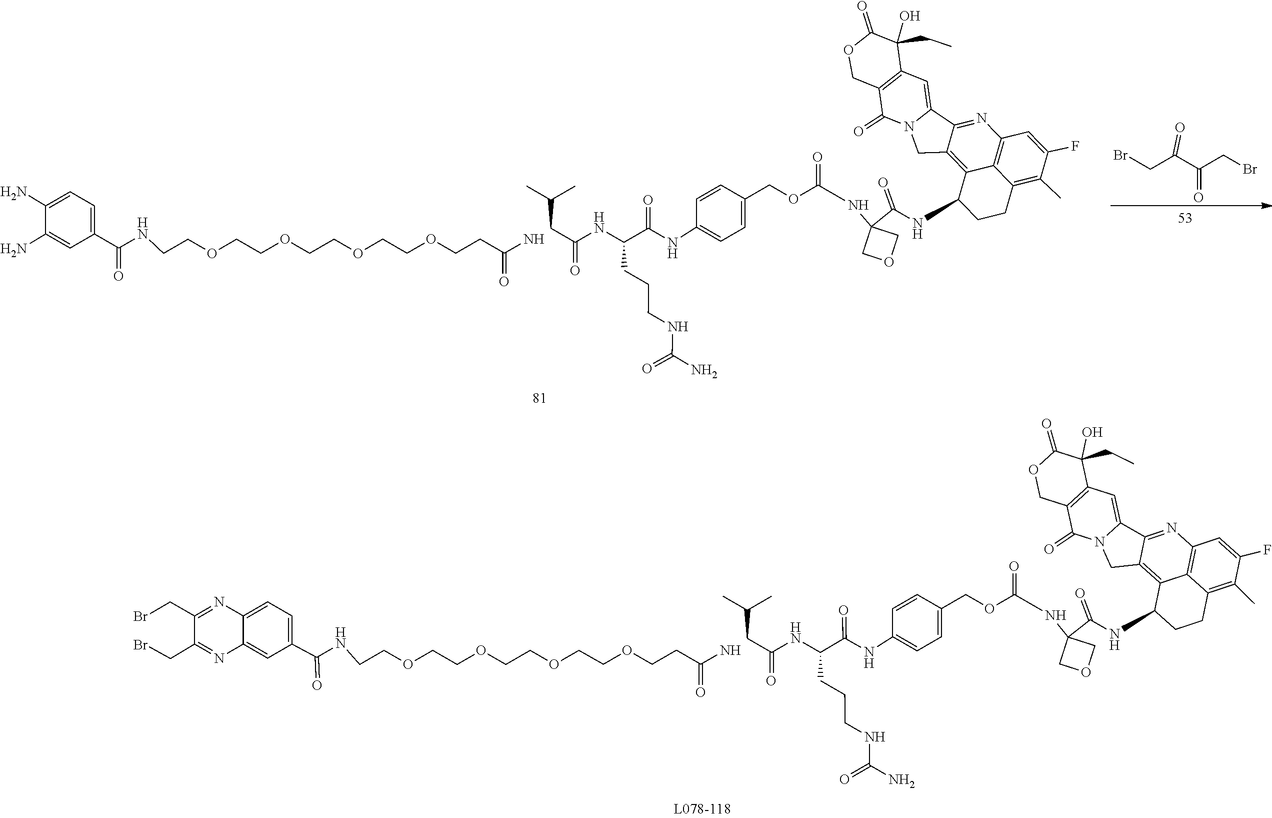

To a solution of compounds L078-088 (TFA salt, 40 mg, 0.062 mmol), 75 (71 mg, 0.093 mmol) and HOBt (5 mg) in 3 mL of DMF was added DIEA (16 mg, 0.124 mmol). The solution was stirred for 16 hours. To the solution was added 0.5 ml of diethylamine and stirred for 30 minutes. The mixture was purified by HPLC to give compound 80 (TFA salt, 59 mg).

To a solution of compound 80 (TFA salt, 59 mg, 0.056 mmol), compound 77 (47 mg, 0.056 mmol) and HATU (22 mg, 0.056 mmol) in DMF (3 mL) was added DIEA (19 mg, 0.147 mmol). The solution was stirred for 5 minutes followed by addition of 0.5 mL of diethylamine. The mixture was stirred for another 30 minutes, purified by HPLC and dried to give compound 81 (TFA salt, 36 mg).

Compound 81 (TFA salt, 36 mg) was dissolved in 3 ml of 60% acetonitrile/H₂O (1% TFA). To the solution was added a solution of 53 (10 mg) in acetonitrile, stirred for 5 minutes and purified by HPLC to give compound L078-118 (11 mg), MS m/z 1529.5 (M+H).

### Example S30: Synthesis of Compound L078-119

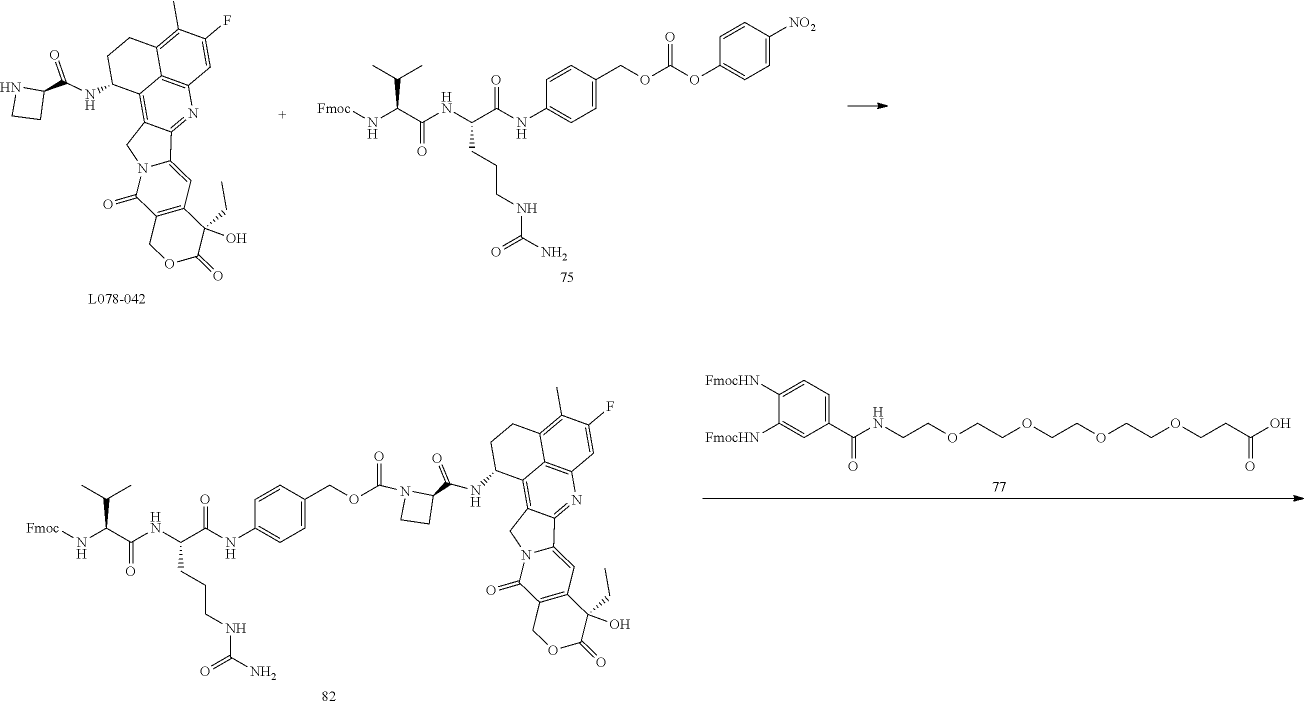

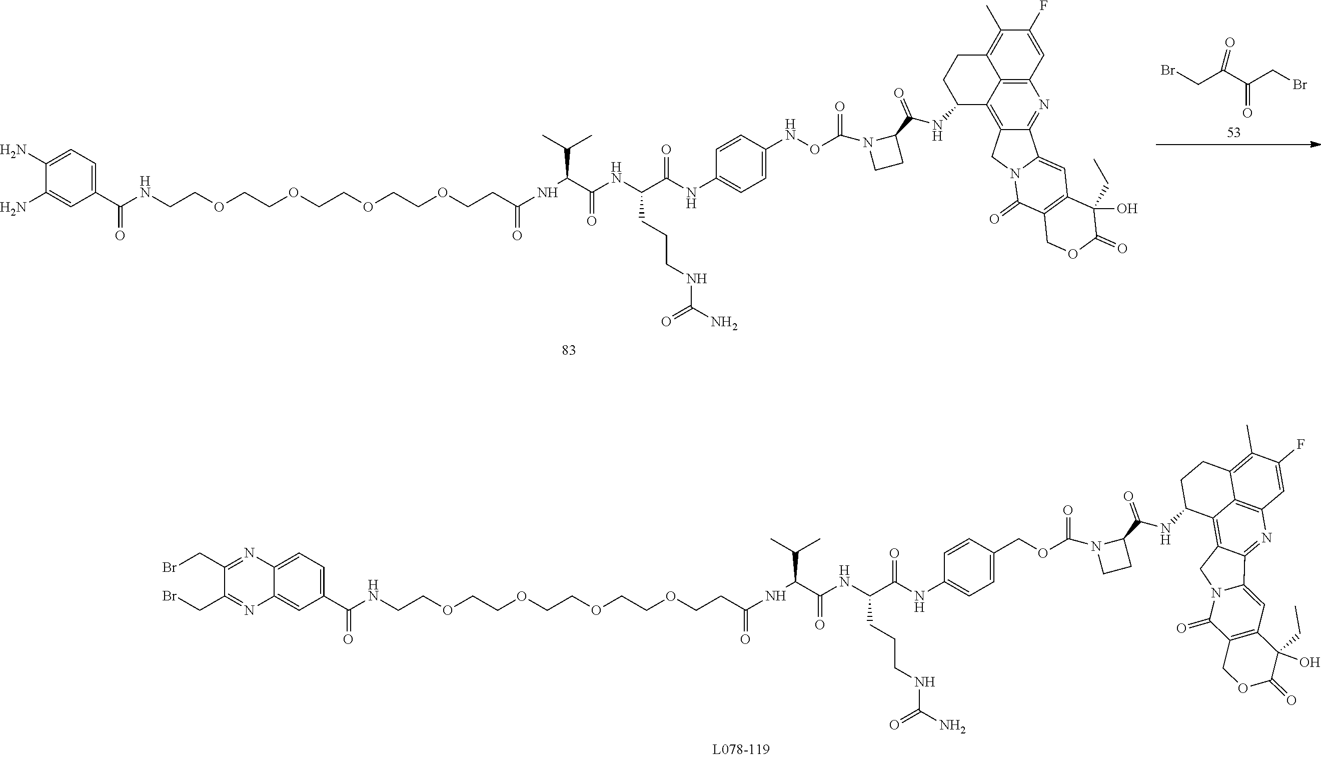 [SMILES: CC[C@]1(O)C(=O)OCc2c1cc1n(c2=O)Cc2c-1nc1cc(F)c(C)c3c1c2[C@H](NC(=O)[C@H]1CCN1C(=O)ONc1ccc(NC(=O)[C@H](CCCNC(N)=O)NC(=O)[C@@H](NC(=O)CCOCCOCCOCCOCCNC(=O)c2ccc(N)c(N)c2)C(C)C)cc1)CC3 ; O=C(CBr)C(=O)CBr ; CC[C@]1(O)C(=O)OCc2c1cc1n(c2=O)Cc2c-1nc1cc(F)c(C)c3c1c2[C@H](NC(=O)[C@H]1CCN1C(=O)OCc1ccc(NC(=O)[C@H](CCCNC(N)=O)NC(=O)[C@@H](NC(=O)CCOCCOCCOCCOCCNC(=O)c2ccc4nc(CBr)c(CBr)nc4c2)C(C)C)cc1)CC3]

A solution of compound L078-042 (TFA salt, 50 mg, 0.079 mmol), compound 75 (60 mg, 0.078 mmol), HOBT (5 mg) and DIEA (20 mg, 0.158 mmol) in DMF (3 mL) was stirred for 2 hours. To the solution was added 0.5 ml of diethylamine and the solution was stirred for another 30 minutes. The mixture was purified by HPLC to give compound 82 (58 mg). To a solution of compound 82 (58 mg, 0.056 mmol), compound 77 (47 mg, 0.056 mmol) and HATU (22 mg, 0.056 mmol) in DMF (3 mL) was added DIEA (19 mg, 0.147 mmol). The solution was stirred for 5 minutes followed by addition of 0.5 mL of diethylamine. The mixture was stirred for additional 30 minutes and purified by HPLC and dried to give compound 83 (52 mg). Compound 83 (52 mg) was dissolved in 3 ml of 60% CH₃CN/H₂O (1% TFA). To the solution was added a solution of compound 53 (10 mg) in CH₃CN, stirred for 5 minutes and purified by HPLC to give compound L078-119 (30 mg), MS m/z 1513.28 (M+H).

### Example S31: Synthesis of Compound L078-120

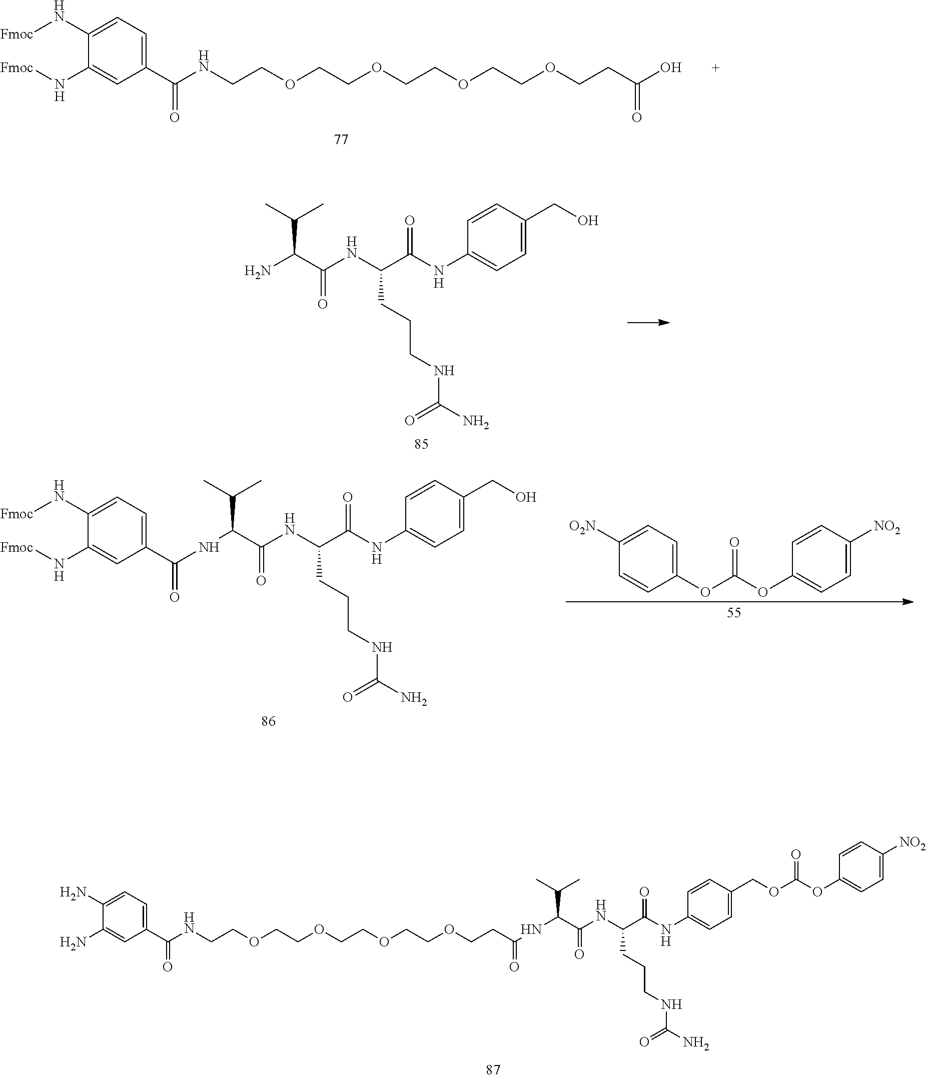

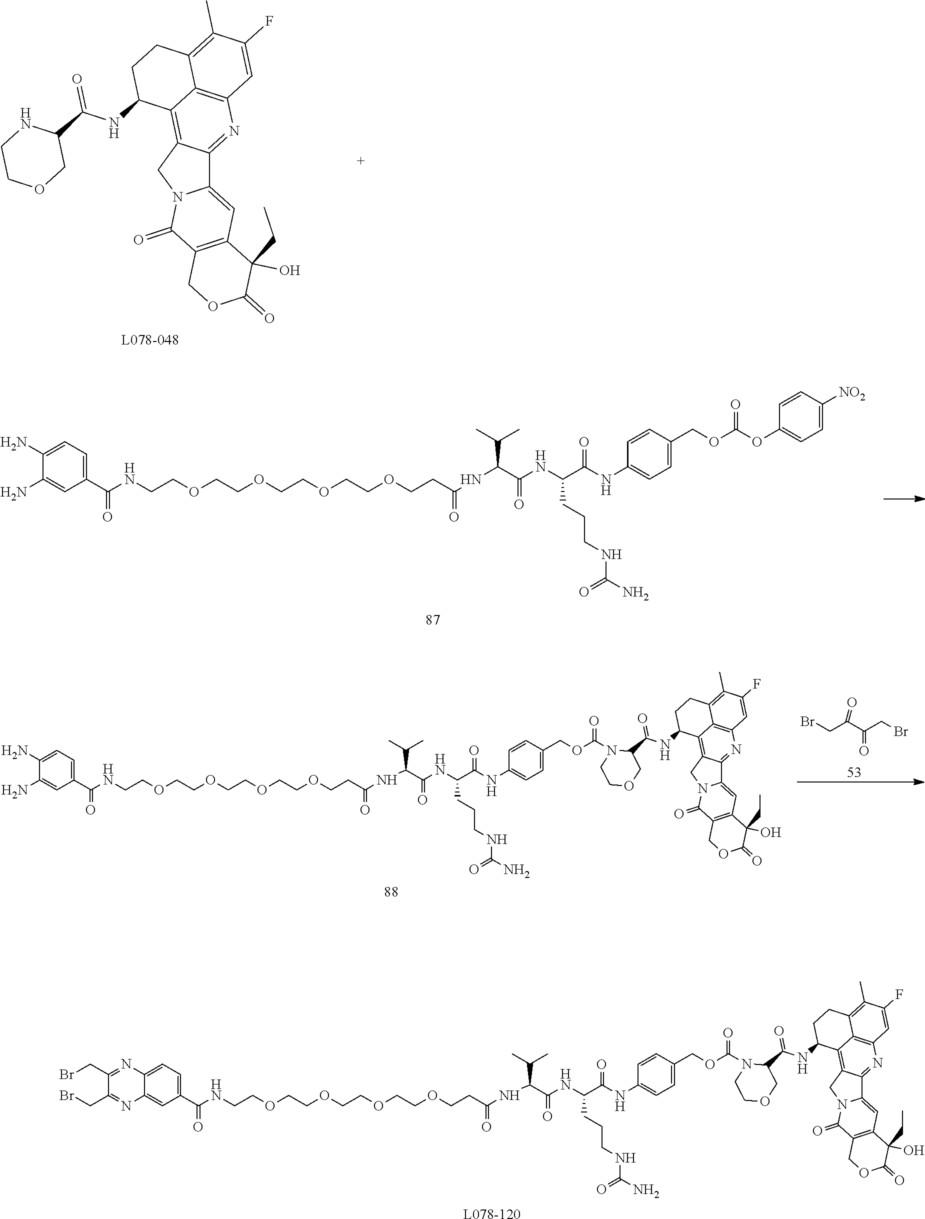

A solution of compound 77 (400 mg, 0.474 mmol), TFA salt of compound 85 (CAS #159857-79-1; 180 mg, 0.474 mmol), HATU (180 mg, 0.474 mmol) and DIEA (122 mg, 0.946 mmol) in DMF (5 mL) was stirred for 5 minutes to give compound 86. Compound 55 (216 mg, 0.711 mmol) was added to the crude solution of compound 86 and the mixture was stirred for another 4 hours. The mixture was purified by HPLC to give compound 87 (123 mg).

A solution of compound L078-048 (TFA salt, 40 mg, 0.060 mmol), compound 87 (TFA salt, 56 mg, 0.060 mmol), HOBT (5 mg) and DIEA (16 mg, 0.12 mmol) was stirred for 2 hours and purified by HPLC to give compound 88 (28 mg). Compound 88 was dissolved in 3 ml of 60% CH₃CN/H₂O (1% TFA). To the solution was added a solution of compound 53 (10 mg) in CH₃CN, stirred for 5 minutes and purified by HPLC to give compound L078-120 (24 mg), MS m/z 1543.7 (M+H).

### Example S32: Synthesis of Compound L078-177

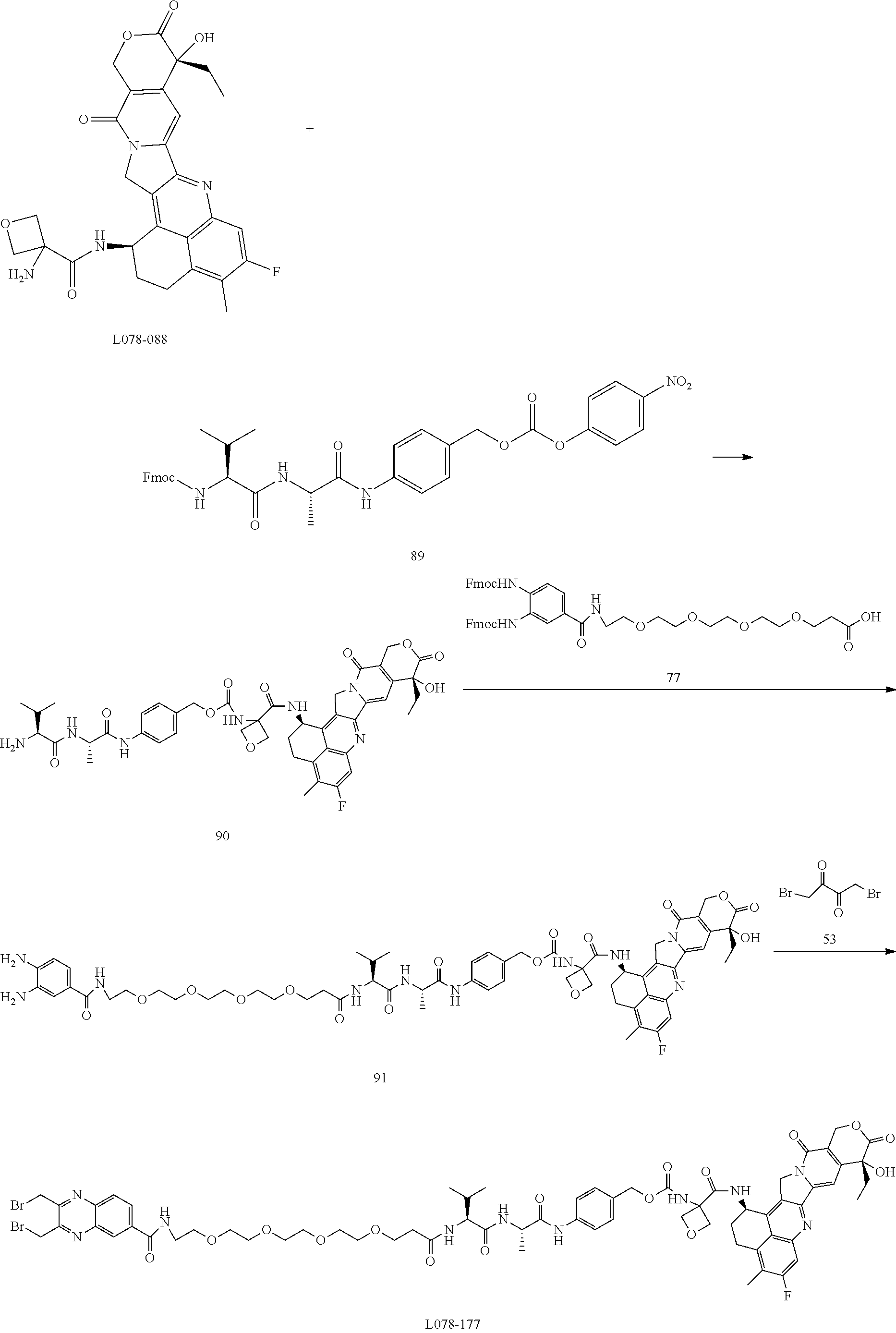

A solution of compound L078-088 (TFA salt, 30 mg, 0.046 mmol), compound 89 (CAS #1394238-92-6; 38 mg, 0.055 mmol), HOBT (5 mg) and DIEA (12 mg, 0.092 mmol) in DMF (3 mL) was stirred for 16 hours, followed by addition of 0.5 ml diethylamine, and stirred for another 30 minutes. The mixture was purified by HPLC to give compound 90 (20 mg). To a solution of compound 90 (TFA salt, 20 mg, 0.021 mmol), compound 77 (18 mg, 0.021 mmol) and HATU (8 mg, 0.021 mmol) in DMF (3 mL) was added DIEA (7 mg, 0.052 mmol). The solution was stirred for 5 minutes followed by addition of 0.5 mL of diethylamine. The mixture was stirred for additional 30 minutes and purified by HPLC to give compound 91. Compound 91 was dissolved in 3 ml of 60% CH₃CN/H₂O (1% TFA). To the solution was added a solution of compound 53 (6 mg) in CH₃CN, stirred for 5 minutes and purified by HPLC to give compound L078-118 (4 mg), MS m/z 1442.4 (M+H).

### Example S33: Synthesis of Compound L078-130

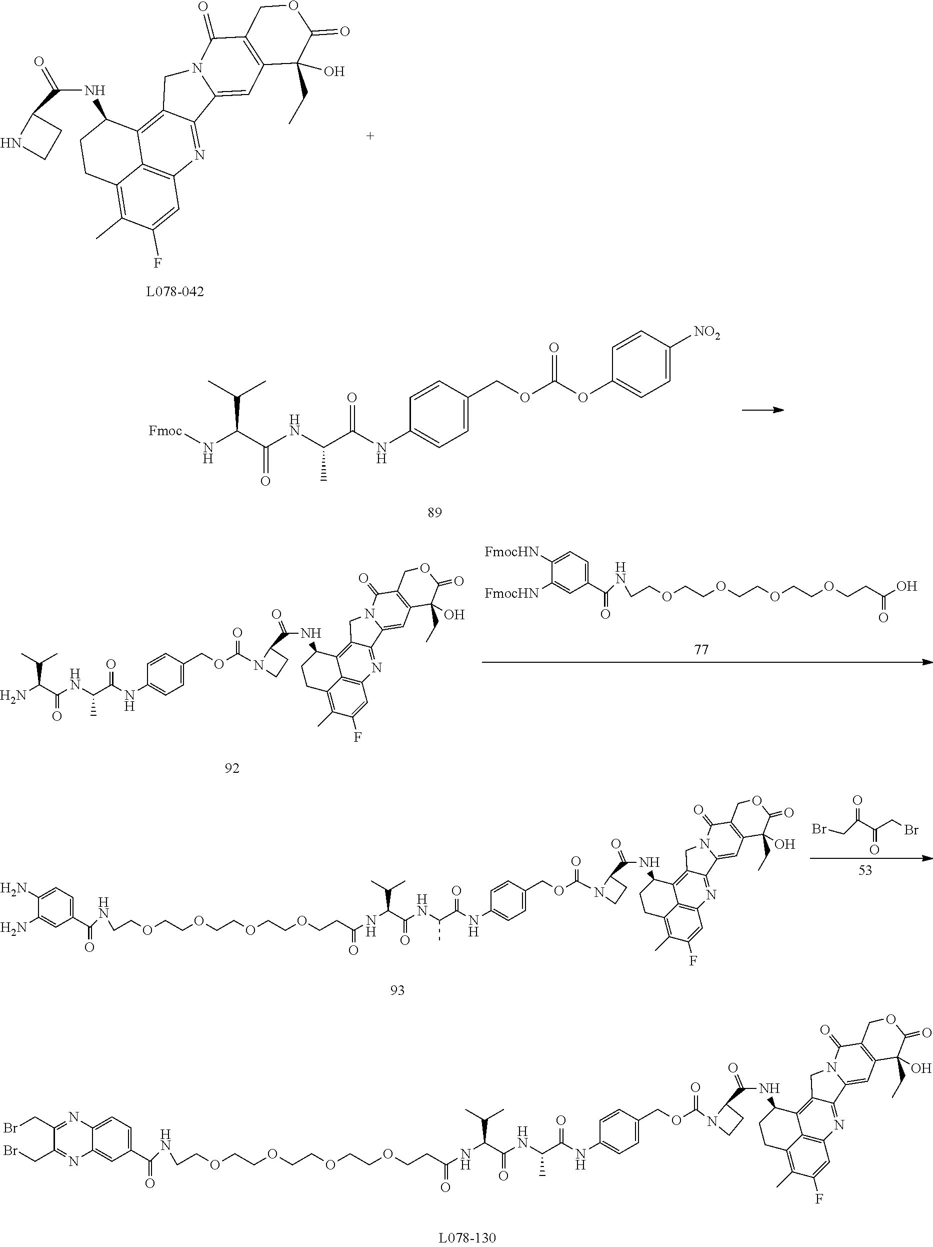

A solution of compound L078-042 (TFA salt, 30 mg, 0.047 mmol), compound 89 (40 mg, 0.059 mmol), HOBT (5 mg) and DIEA (12 mg, 0.94 mmol) in DMF (3 mL) was stirred for 2 hours. To the solution was added 0.5 ml of diethylamine and stirred for additional 30 minutes. The mixture was purified by HPLC to give compound 92. To a solution of compound 92 (TFA salt, 45 mg, 0.047 mmol), compound 77 (40 mg, 0.047 mmol) and HATU (18 mg, 0.047 mmol) in DMF (3 mL) was added DIEA (15 mg, 0.117 mmol). The solution was stirred for 5 minutes followed by addition of 0.5 mL diethylamine. The mixture was then stirred for additional 30 minutes, purified by HPLC, and dried to give compound 93. Compound 93 was dissolved in 3 ml of 60% CH₃CN/H₂O (1% TFA). To the solution of compound 93 was added a solution of compound 53 (10 mg) in CH₃CN, stirred for 5 minutes and purified by HPLC to give compound L078-130 (18 mg), MS m/z 1427.7 (M+H).

### Example S34: Synthesis of Compound L078-123

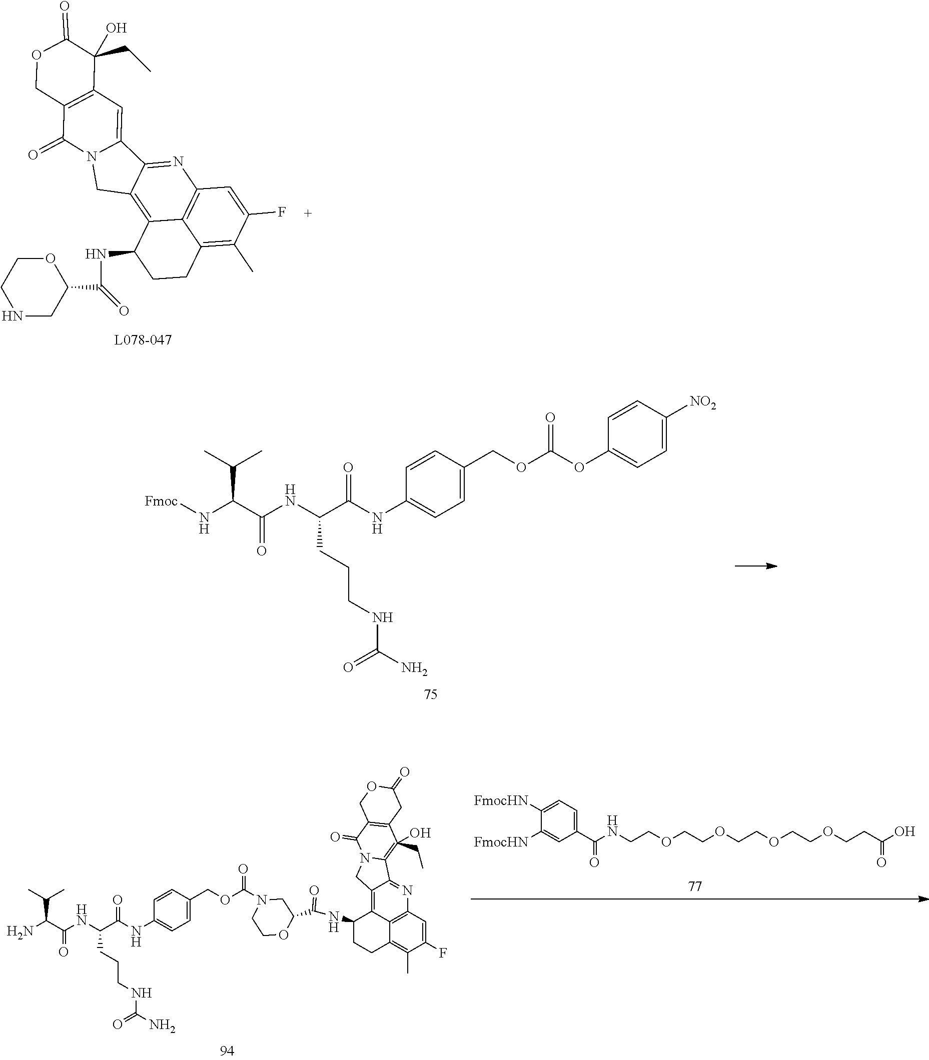

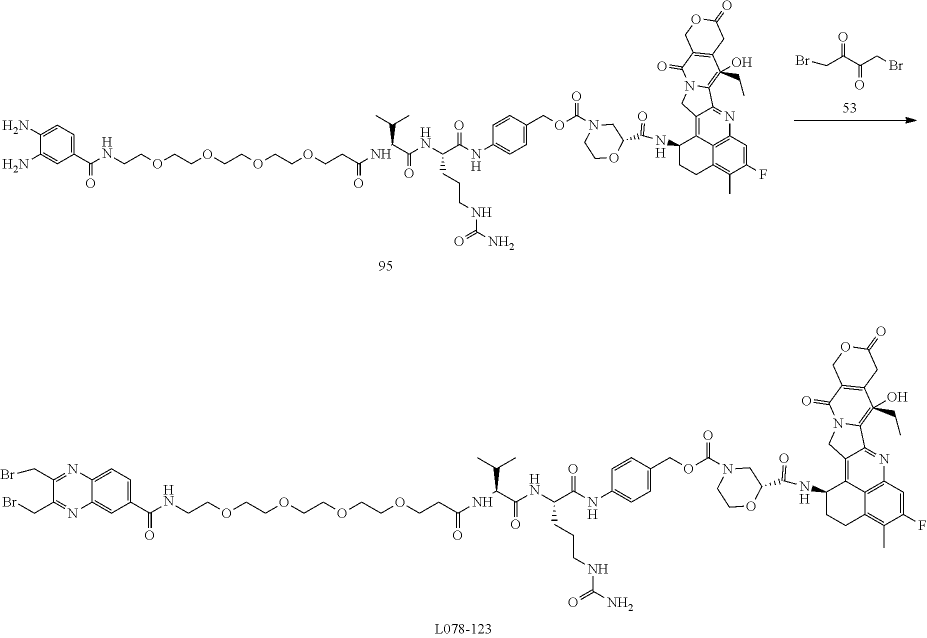

A solution of compound L078-047 (TFA salt, 50 mg, 0.075 mmol), compound 75 (86 mg, 0.112 mmol), HOBT (5 mg) and DIEA (19 mg, 0.15 mmol) in DMF (3 mL) was stirred for 2 hours. To the solution was added 0.5 ml of diethylamine and the solution was stirred for additional 30 minutes. The mixture was purified by HPLC to give compound 94 (62 mg). To a solution of compound 94 (TFA salt, 62 mg, 0.058 mmol), compound 77 (49 mg, 0.058 mmol) and HATU (22 mg, 0.058 mmol) in DMF (3 mL) was added DIEA (19 mg, 0.147 mmol). The solution was stirred for 5 minutes followed by addition of 0.5 mL of diethylamine. The mixture was stirred for 30 minutes and purified by HPLC and dried to give compound 95. Compound 95 was dissolved in 3 ml of 60% CH₃CN/H₂O (1% TFA). To the solution of compound 95 was added a solution of compound 53 (10 mg) in CH₃CN, stirred for 5 minutes and purified by HPLC to give compound L078-123 (13 mg), MS m/z 1543.3 (M+H).

### Example S35: Synthesis of Compound L078-139

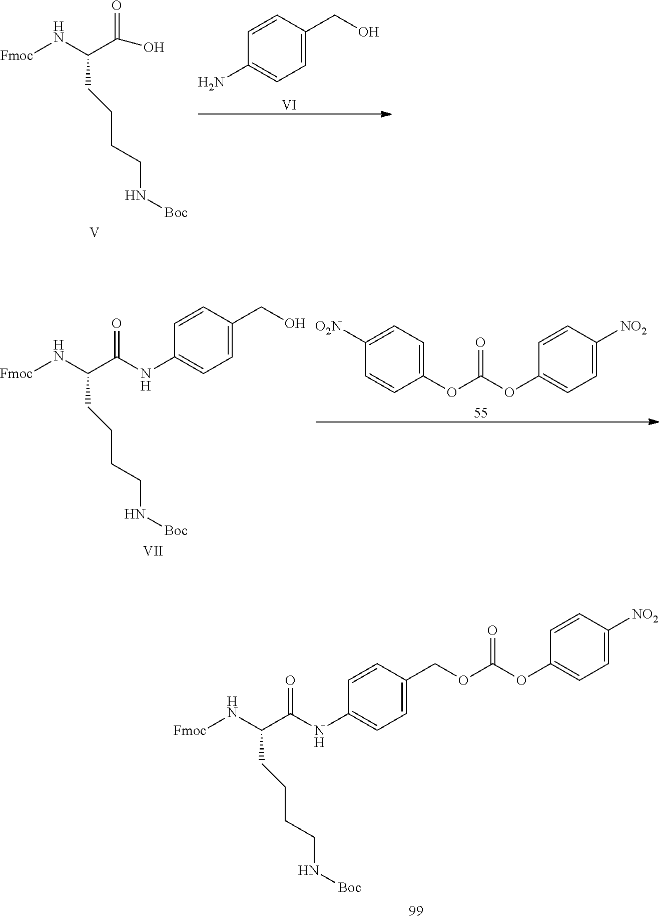

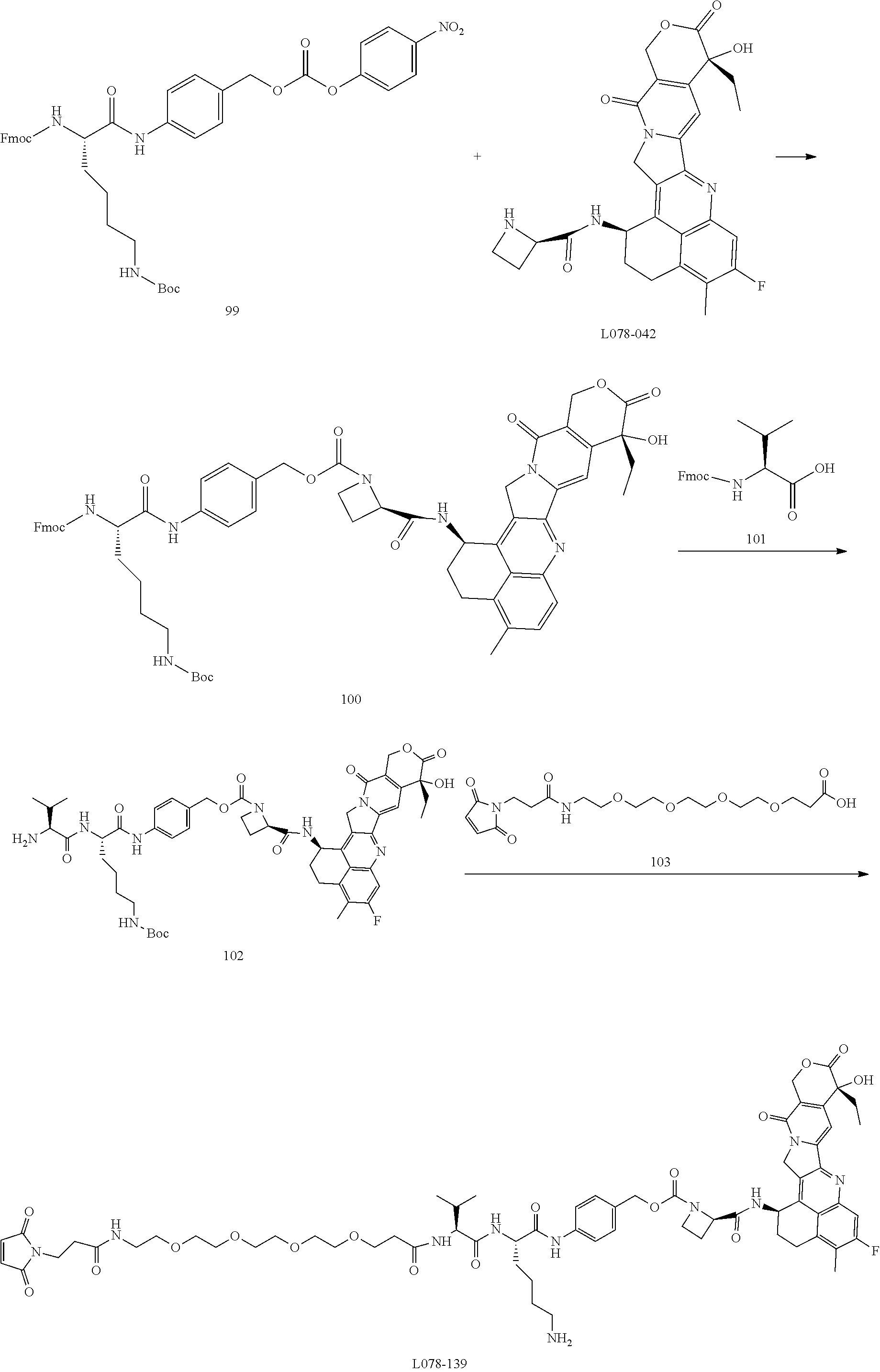

A solution of compound V (1 g, 2.13 mmol), compound VI (CAS #5070-13-3; 288 mg, 2.34 mmol) and EEDQ (790 mg, 3.19 mmol) in dichloromethane was stirred for 2 hours. The solution was concentrated and purified by HPLC to give compound VII (1.1 g). A solution of compound VII (1 g, 1.916 mmol), compound 55 (873 mg, 2.874 mmol) and DIEA (258 mg, 2 mmol) in DMF (10 mL) was stirred for 5 hours. The crude was purified by HPLC to give compound 99 (823 mg).

A solution of compound L078-042 (TFA salt, 50 mg, 0.079 mmol), compound 99 (64 mg, 0.087 mmol), HOBT (5 mg) and DIEA (20 mg, 0.16 mmol) in DMF (3 mL) was stirred for 2 hours followed by addition of 0.5 ml of diethylamine, and then stirred for another 30 minutes. The mixture was purified by HPLC to give compound 100 (58 mg). To a solution of compound 100 (TFA salt, 58 mg, 0.057 mmol), compound 101 (20 mg, 0.057 mmol) and HATU (21 mg, 0.057 mmol) in DMF (3 mL) was added DIEA (19 mg, 0.144 mmol). The solution was stirred for 5 minutes followed by addition of 0.5 mL of diethylamine. The mixture was stirred for additional 30 minutes, purified by HPLC and dried to give compound 102 (16 mg). To a solution of compound 102 (TFA salt, 16 mg, 0.014 mmol), compound 103 (6 mg, 0.014 mmol) and HATU (6 mg, 0.016 mmol) was added DIEA (5 mg, 0.035 mmol). After being stirred for 5 minutes the crude was purified by HPLC and dried. The residue was treated with 30% TFA in dichloromethane (1 mL) for 30 minutes and purified by HPLC to give L078-139 (TFA salt, 4 mg), MS m/z 1293.4 (M+H).

### Example S36: Synthesis of Compound L078-163

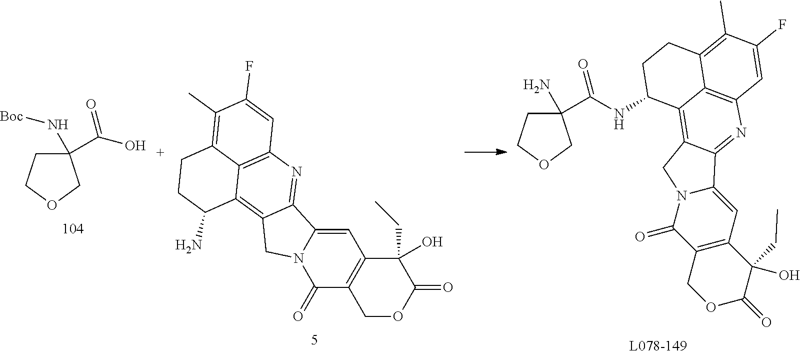 [SMILES: CC(C)(C)OC(=O)NC1(C(=O)O)CCOC1 ; CC[C@]1(O)C(=O)OCc2c1cc1n(c2=O)Cc2c-1nc1cc(F)c(C)c3c1c2[C@H](N)CC3 ; CC[C@]1(O)C(=O)OCc2c1cc1n(c2=O)Cc2c-1nc1cc(F)c(C)c3c1c2[C@H](NC(=O)C1(N)CCOC1)CC3]

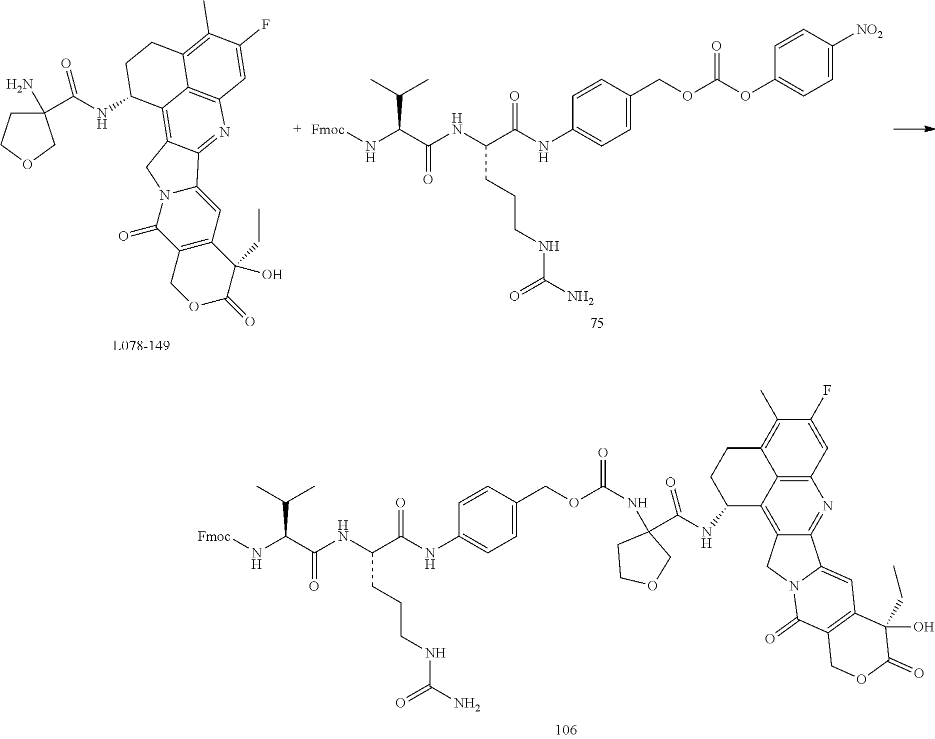

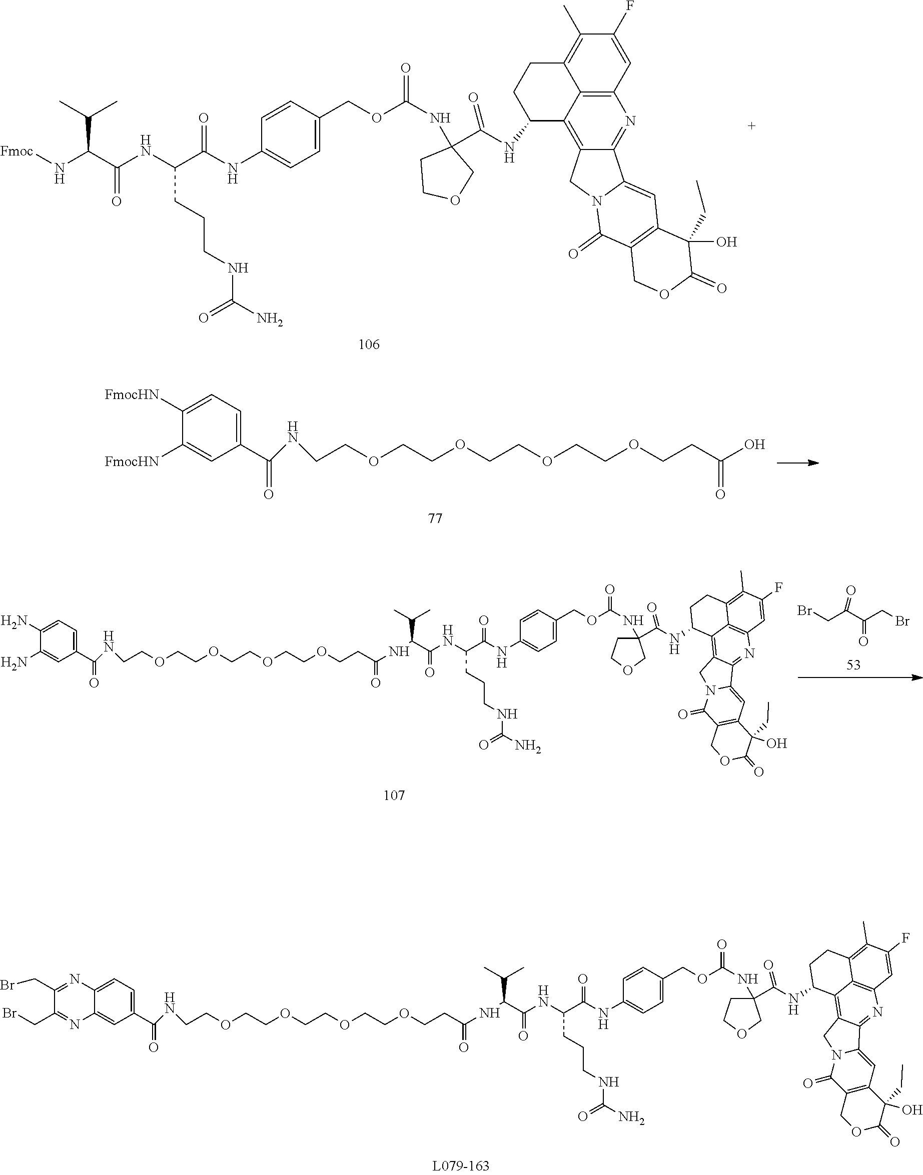

To a solution of compound 104 (44 mg, 0.188 mmol), compound 5 (mesylate salt, 100 mg, 0.188 mol) and HATU (71 mg, 0.188 mmol) in 3 mL of DMF was added DIEA (49 mg, 0.376 mol). The solution was stirred for 5 minutes and purified by HPLC. The resulting product was treated with 50% TFA in dichloromethane (1 mL) for 30 minutes and concentrated to give compound L078-149 (TFA salt, 130 mg) as a yellow powder. MS m/z 548.4 (M+H).

A solution of L078-149 (TFA salt, 30 mg, 0.045 mmol), compound 75 (50 mg, 0.065 mmol), HOBT (5 mg) and DIEA (12 mg, 0.09 mmol) in DMF (3 mL) was stirred for 16 hours followed by addition of diethylamine (0.5 mL) and stirred for another 30 minutes. The mixture was purified by HPLC to give compound 106 (28 mg). To a solution of compound 106 (28 mg, 0.026 mmol), compound 77 (23 mg, 0.026 mmol) and HATU (10 mg, 0.026 mmol) in DMF (3 mL) was added DIEA (9 mg, 0.065 mmol). The solution was stirred for 5 minutes followed by addition of 0.5 mL of diethylamine. The mixture was stirred for another 30 minutes, purified by HPLC and dried to give compound 107. Compound 107 was dissolved in 3 ml of 60% CH₃CN/H₂O (1% TFA). To the solution was added a solution of compound 53 (8 mg) in CH₃CN, stirred for 5 minutes and purified by HPLC to give compound L078-163 (7 mg), MS m/z 772.2 (M/2+H).

### Example S37: Synthesis of Compound L078-164

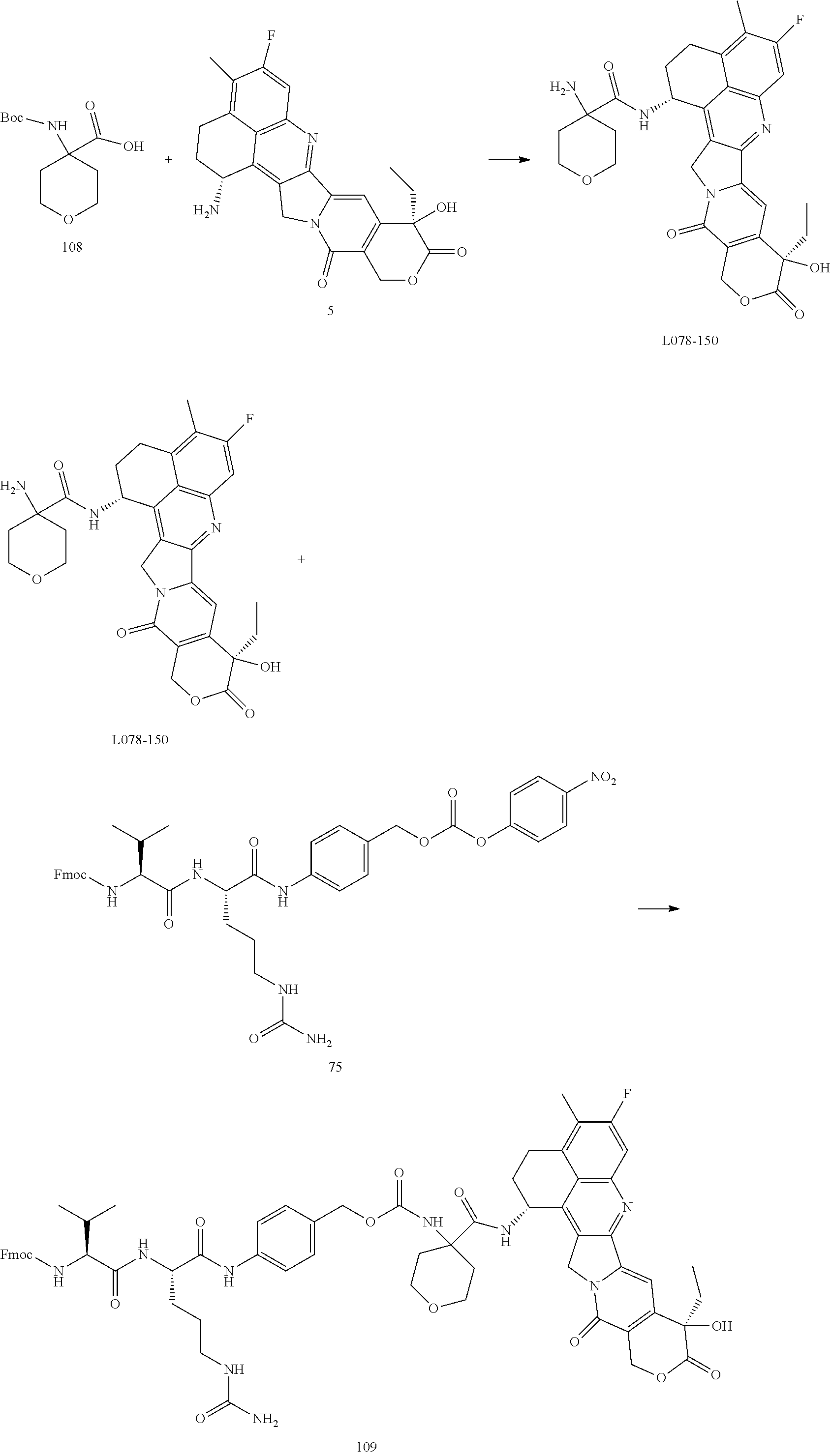

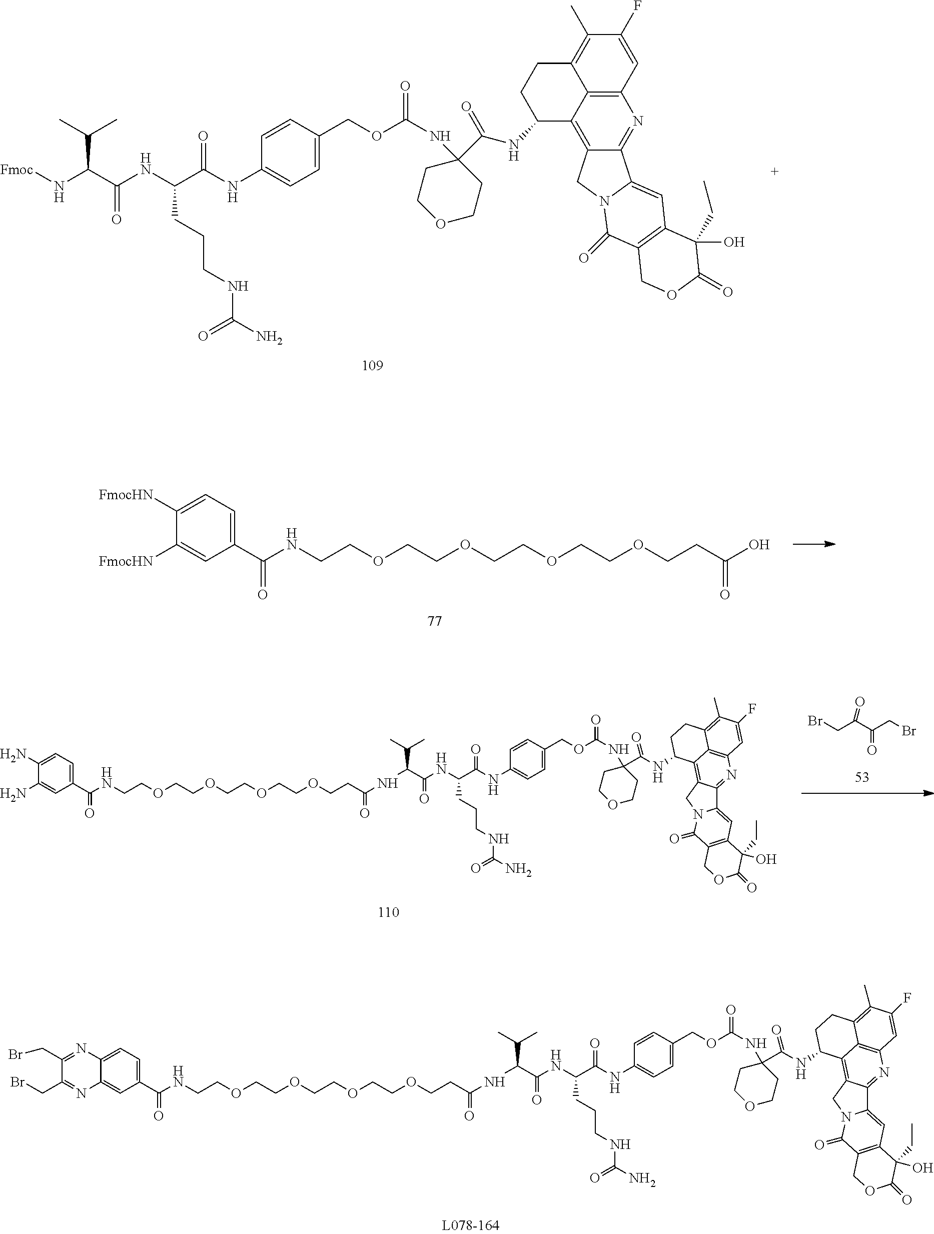

To a solution of compound 108 (23 mg, 0.094 mmol), compound 5 (mesylate salt, 5 mg, 0.094 mol) and HATU (36 mg, 0.094 mmol) in 3 mL of DMF was added DIEA (30 mg, 0.235 mol). The solution was stirred for 5 minutes and purified by HPLC. The resulting product was treated with 50% TFA in DCM (1 mL) for 30 minutes and concentrated to give compound L078-150 (TFA salt, 57 mg) as a yellow powder. MS m/z 564.2 (M+H).

A solution of compound L078-150 (TFA salt, 27 mg, 0.04 mmol), compound 75 (46 mg, 0.06 mmol), HOBT (5 mg) and DIEA (10 mg, 0.08 mmol) in DMF (3 mL) was stirred for 16 hours followed by addition of diethylamine (0.5 mL) and stirred for another 30 minutes. The mixture was purified by HPLC to give compound 109 (19 mg). To a solution of compound 109 (19 mg, 0.018 mmol), compound 77 (15 mg, 0.018 mmol) and HATU (7 mg, 0.018 mmol) in DMF (3 mL) was added DIEA (6 mg, 0.045 mmol). The solution was stirred for 5 minutes followed by addition of 0.5 mL of diethylamine. The mixture was stirred for another 30 minutes, purified by HPLC and dried to give compound 110. Compound 110 was dissolved in 3 ml of 60% CH₃CN/H₂O (1% TFA). To the solution of compound 110 was added a solution of compound 53 (8 mg) in CH₃CN, stirred for 5 minutes and purified by HPLC to give compound L078-164 (5 mg), MS m/z 1556.2 (M+H).

### Example S38: Synthesis of Compound L078-173

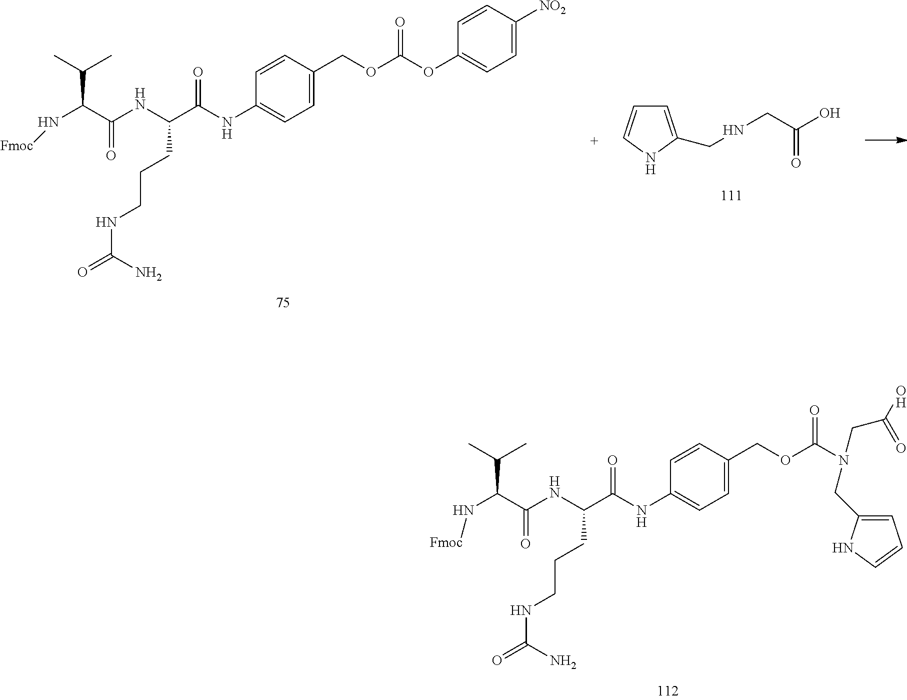

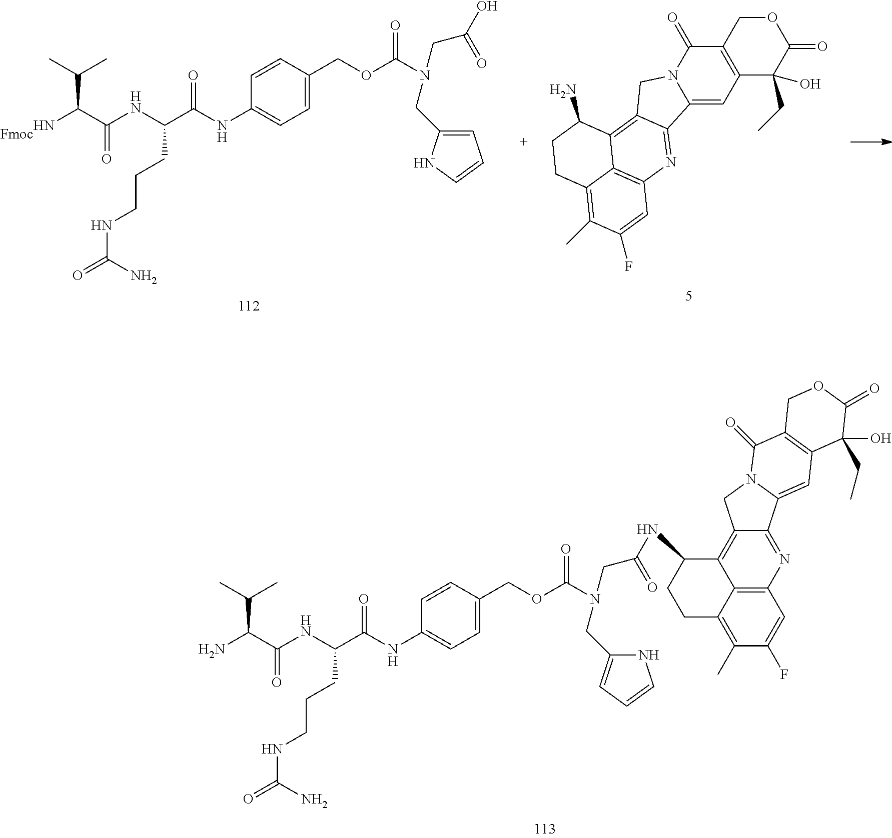

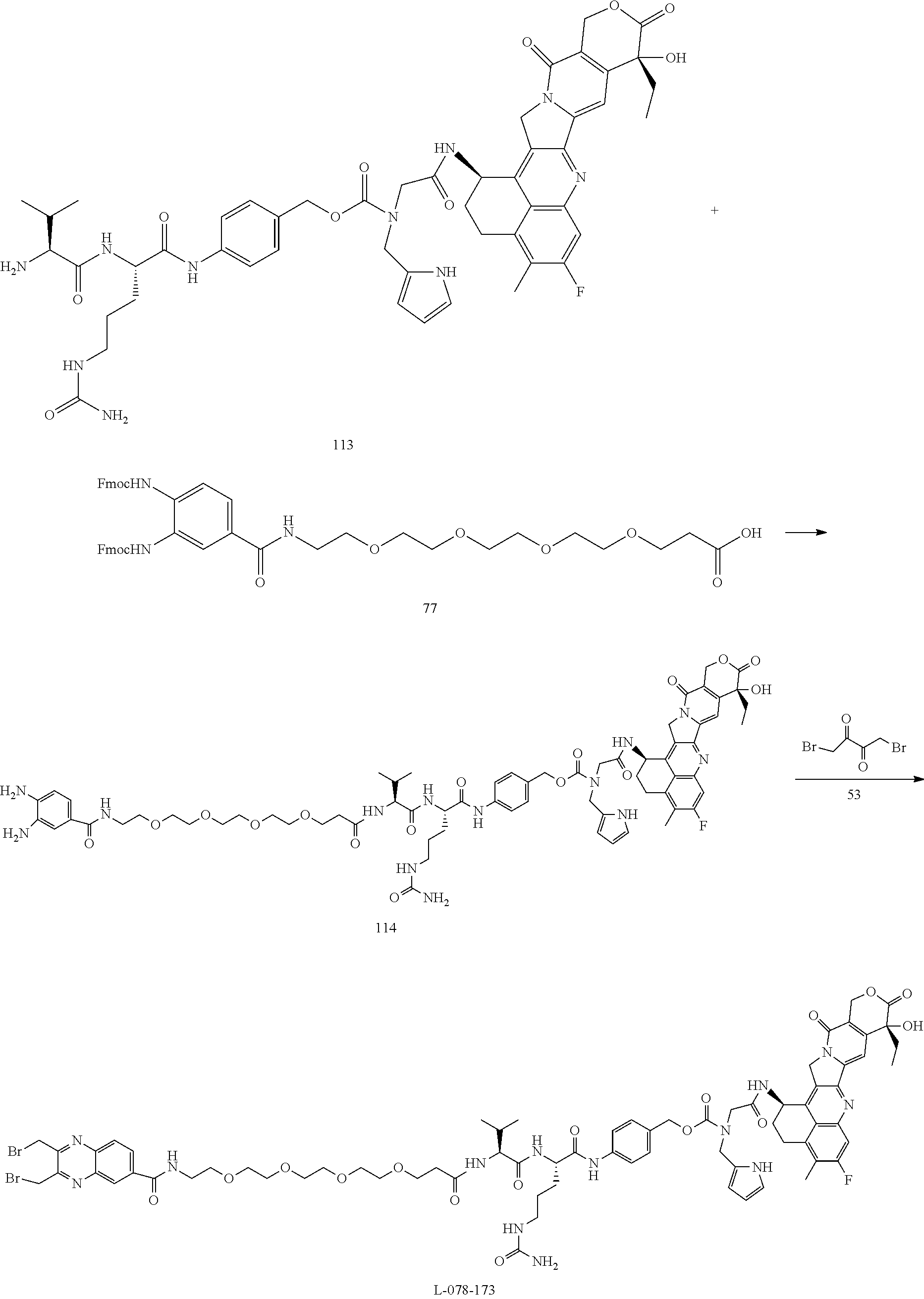

A solution of compound 75 (50 mg, 0.065 mmol), compound 111 (10 mg, 0.065 mmol), HOBT (5 mg) and DIEA (17 mg, 0.143 mmol) in DMF (3 mL) was stirred for 20 minutes. The crude mixture was purified by HPLC to give compound 112 (36 mg). To a solution of compound 112 (36 mg, 0.046 mmol), compound 5 (mesylate salt, 24 mg, 0.046 mmol) and HATU (17 mg, 0.046 mmol) in DMF (2 mL) was added DIEA (12 mg, 0.092 mmol). After being stirred for 5 minutes 0.5 ml of diethylamine was added to the solution and stirred for another 30 minutes. The mixture was purified by HPLC to give compound 113 as a TFA salt. To a solution of compound 113 (TFA salt, 36 mg, 0.046 mmol), compound 77 (39 mg, 0.046 mmol) and HATU (18 mg, 0.046 mmol) in DMF (3 mL) was added DIEA (12 mg, 0.092 mmol). The solution was stirred for 5 minutes followed by addition of 0.5 mL of diethylamine. The mixture was stirred for another 30 minutes, purified by HPLC, and dried to give compound 114. Compound 114 was dissolved in 3 ml of 60% CH₃CN/H₂O (1% TFA). Solution of compound 53 (8 mg) in CH₃CN was added to the solution of compound 114, stirred for 5 minutes and purified by HPLC to give compound L078-173 (13 mg), MS m/z 1567.0 (M+H).

### Example S39: Synthesis of Compound L078-170

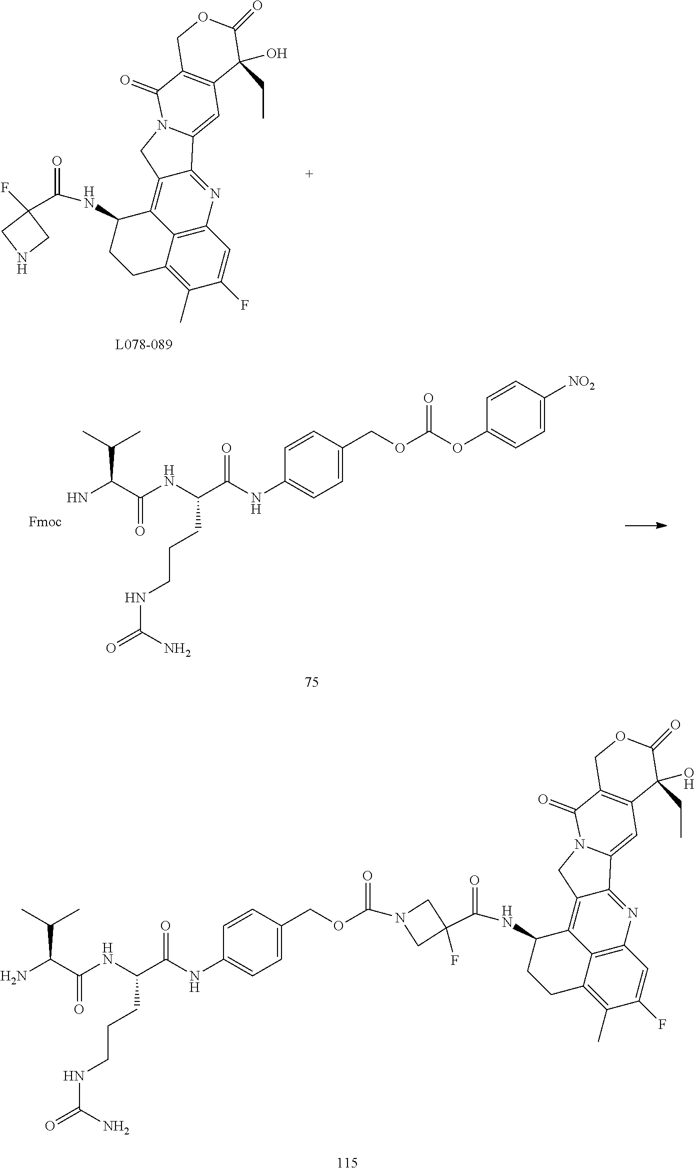

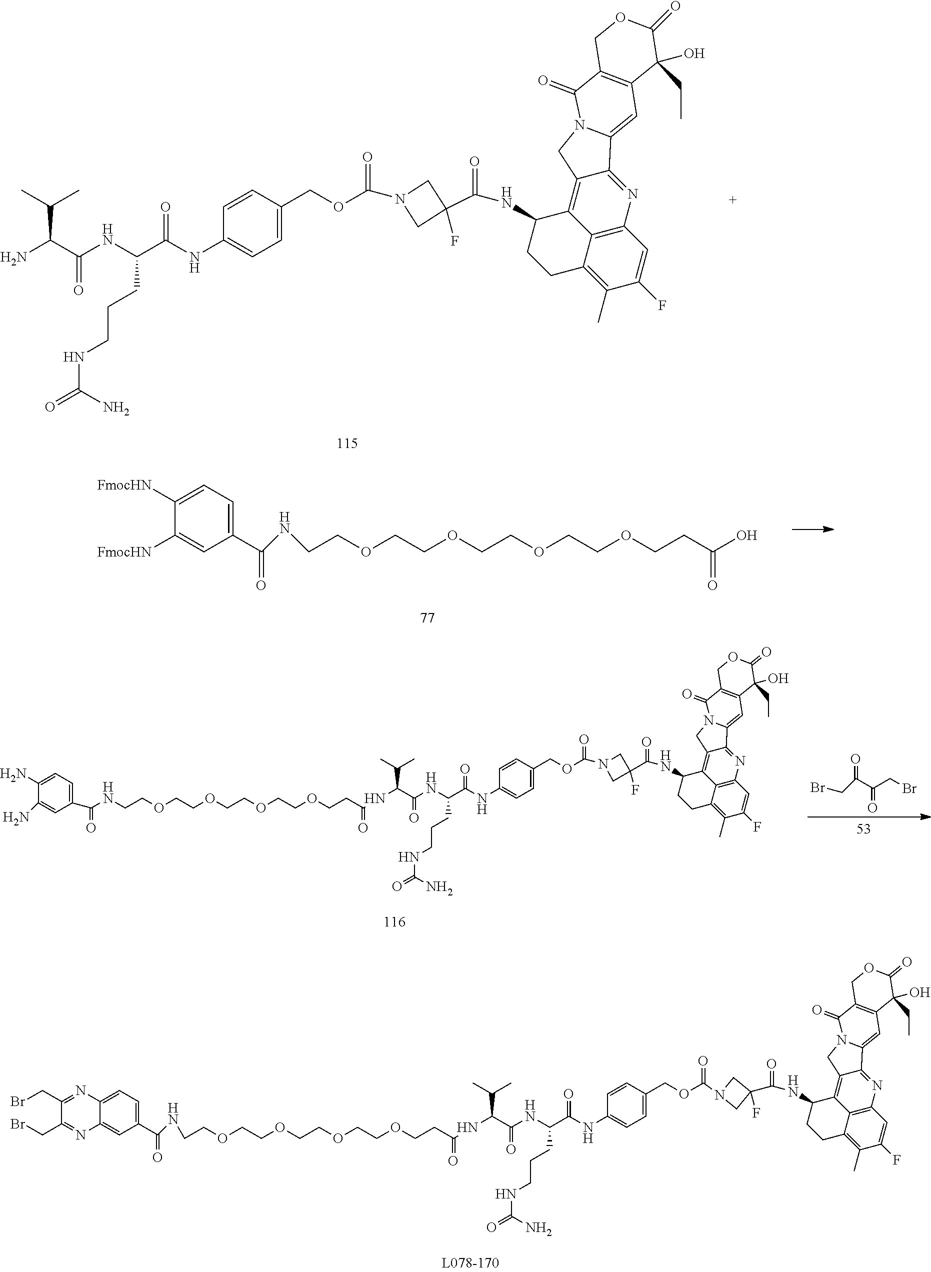

A solution of compound L078-089 (TFA salt, 27 mg, 0.041 mmol), compound 75 (32 mg, 0.041 mmol), HOBT (5 mg) and DIEA (11 mg, 0.082 mmol) in DMF (3 mL) was stirred for 15 hours followed by addition of 0.5 ml of diethylamine and stirred for another 30 minutes. The mixture was purified by HPLC to give compound 115 (28 mg). To a solution of compound 115 (TFA salt, 28 mg, 0.026 mmol), compound 77 (23 mg, 0.026 mmol) and HATU (10 mg, 0.026 mmol) in DMF (3 mL) was added DIEA (9 mg, 0.065 mmol). The solution was stirred for 5 minutes followed by addition of 0.5 mL of diethylamine. The mixture was stirred for another 30 minutes and purified by HPLC to give compound 116. Compound 116 was dissolved in 3 ml of 60% CH₃CN/H₂O (1% TFA). A solution of compound 53 (8 mg) in CH₃CN was added to the solution of compound 116, stirred for 5 minutes and purified by HPLC to give compound L078-170 (6 mg), MS m/z 1531.8 (M+H).

### Example S40: Synthesis of Compound L078-171

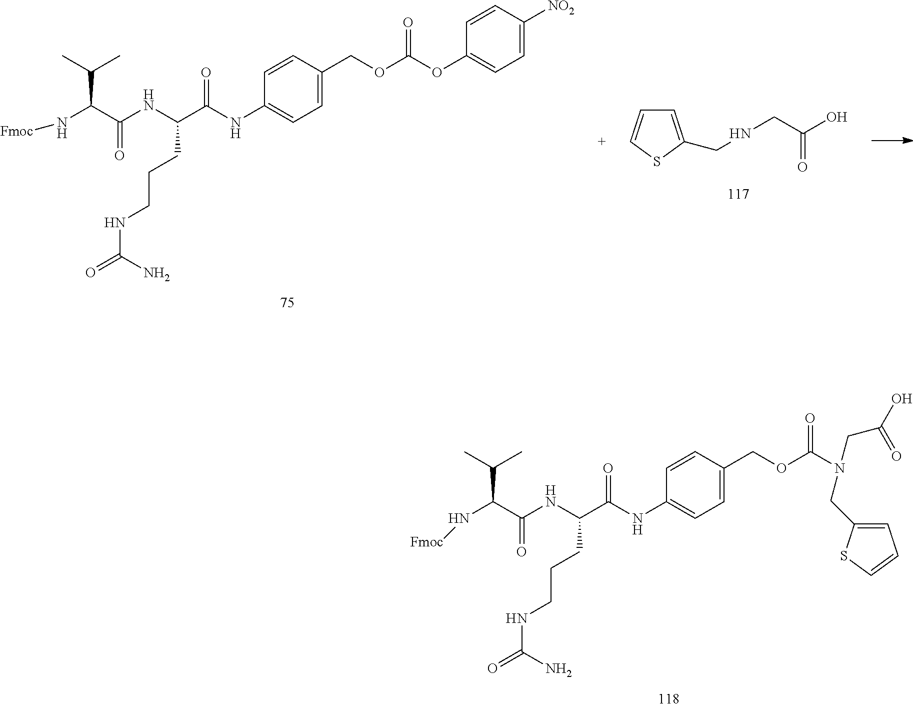

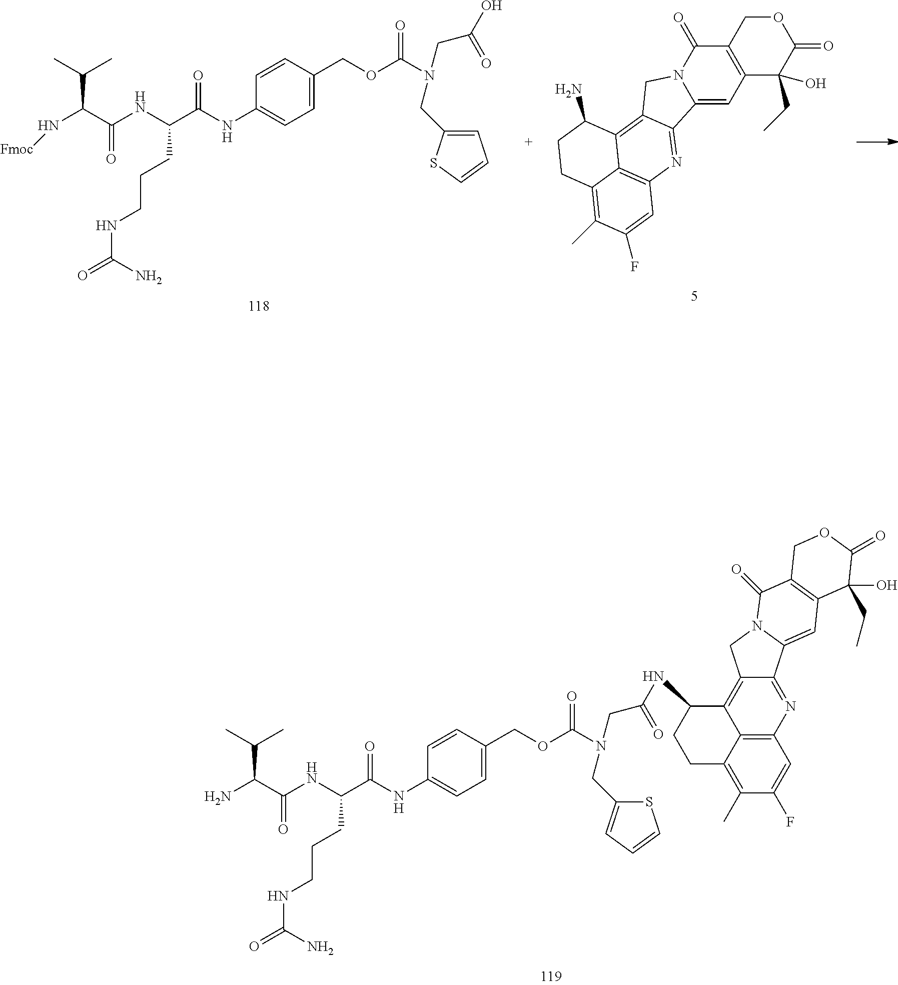

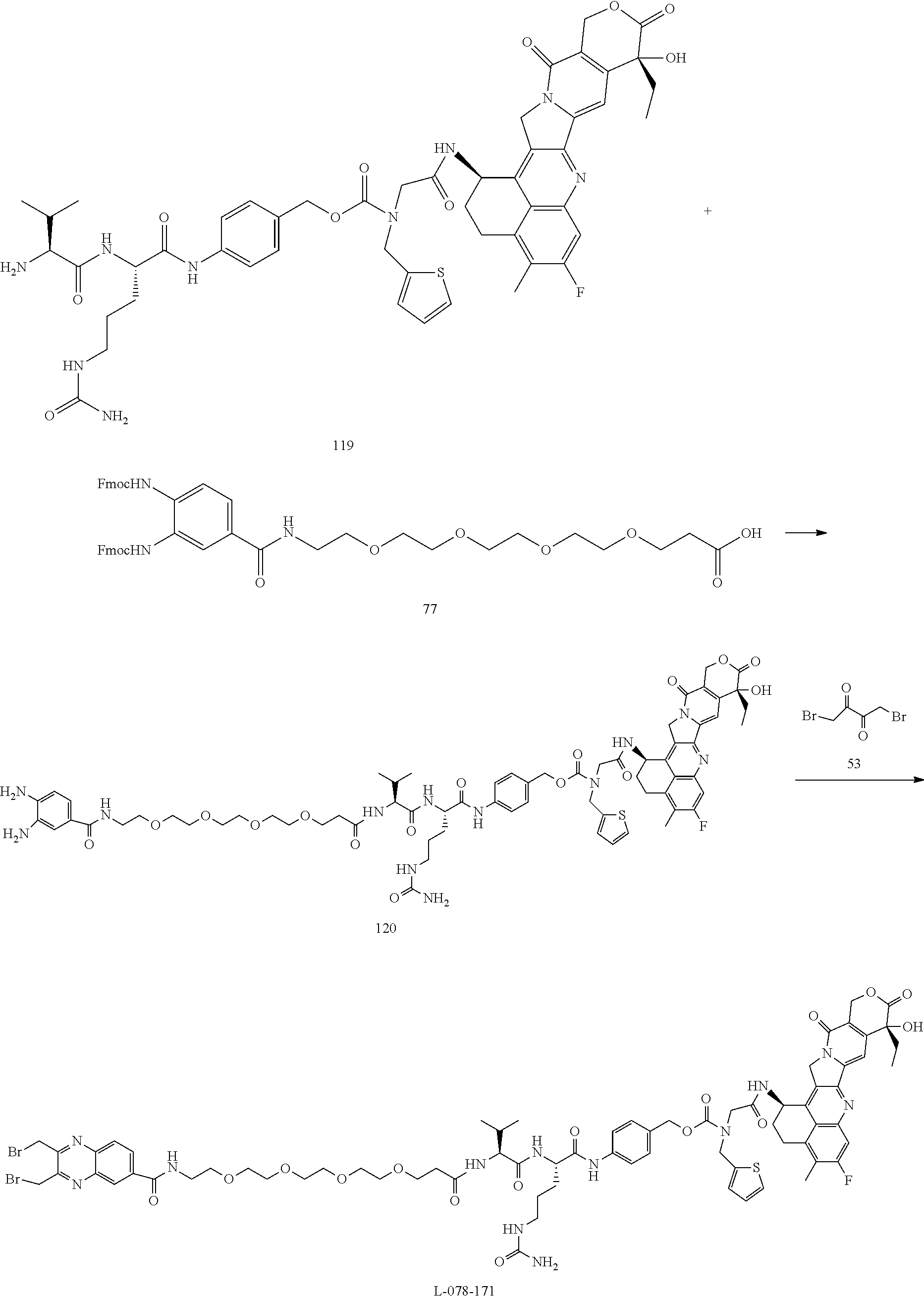

A solution of compound 75 (54 mg, 0.070 mmol), compound 117 (20 mg, 0.070 mmol), HOBT (5 mg) and DIEA (18 mg, 0.14 mmol) in DMF (3 mL) was stirred for 20 minutes. The crude mixture was purified by HPLC to give compound 118 (19 mg). To a solution of compound 118 (19 mg, 0.023 mmol), compound 5 (mesylate salt, 13 mg, 0.023 mmol) and HATU (9 mg, 0.023 mmol) in DMF (2 mL) was added DIEA (8 mg, 0.058 mmol). After being stirred for 5 minutes 0.5 ml of diethylamine was added to the solution and stirred for another 30 minutes. The mixture was purified by HPLC to give compound 119 (20 mg) as a TFA salt. To a solution of compound 119 (TFA salt, 20 mg, 0.018 mmol), compound 77 (16 mg, 0.018 mmol) and HATU (7 mg, 0.018 mmol) in DMF (3 mL) was added DIEA (6 mg, 0.045 mmol). The solution was stirred for 5 minutes followed by addition of 0.5 mL of diethylamine. The mixture was stirred for another 30 minutes, purified by HPLC and dried to give compound 120. Compound 120 was dissolved in 3 ml of 60% CH₃CN/H₂O (1% TFA). A solution of compound 53 (8 mg) in CH₃CN was added solution of compound 120, stirred for 5 minutes and purified by HPLC to give compound L078-171 (7 mg), MS m/z 1583.8 (M+H).

### Example S41: Synthesis of Compound L078-162

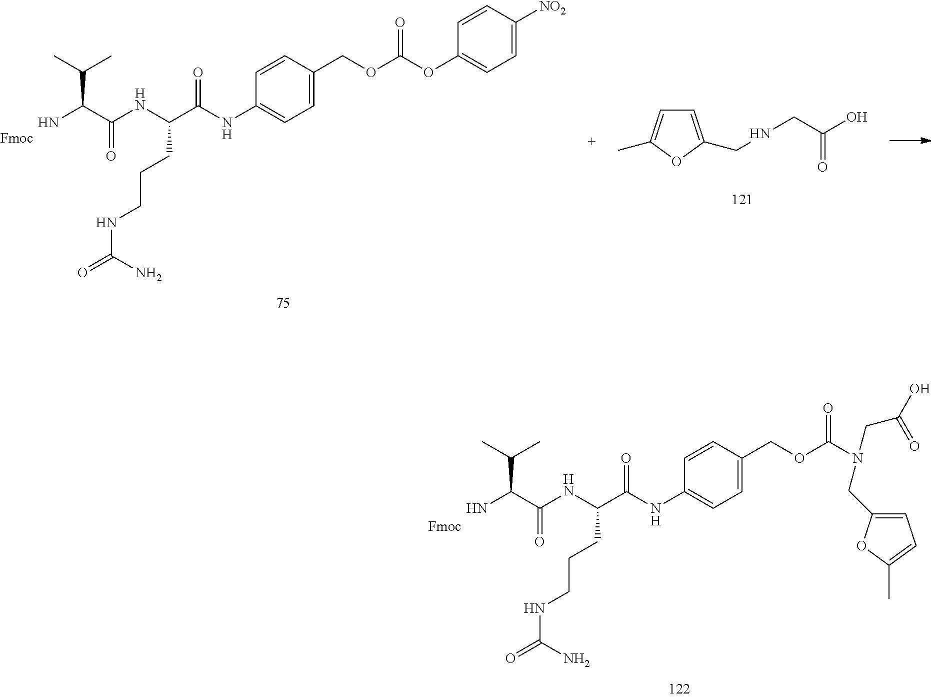

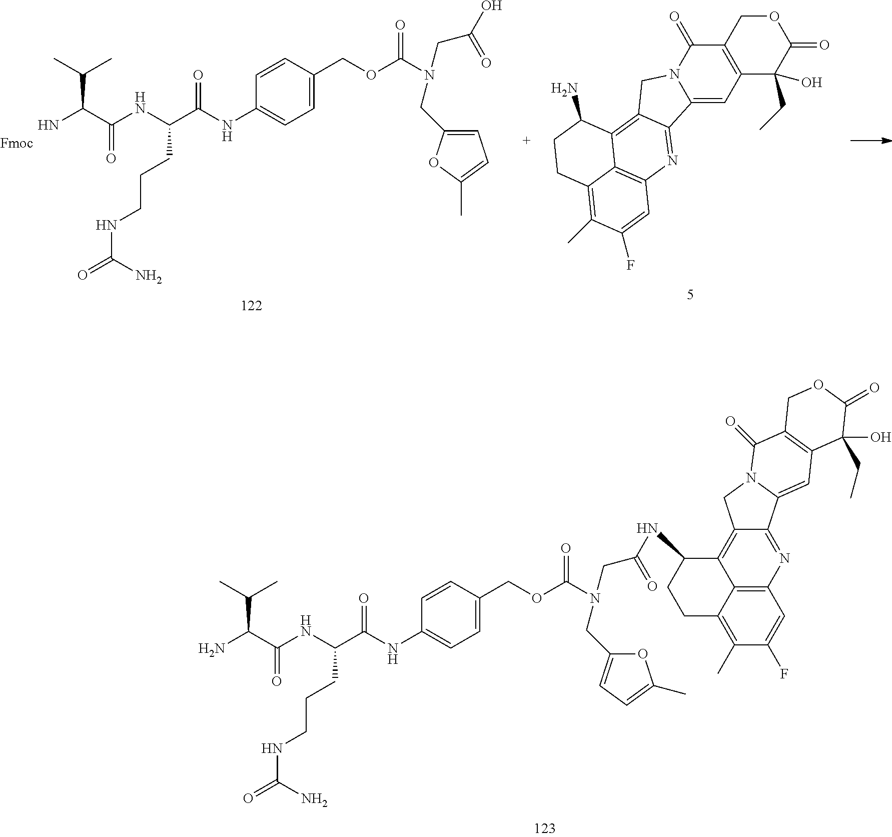

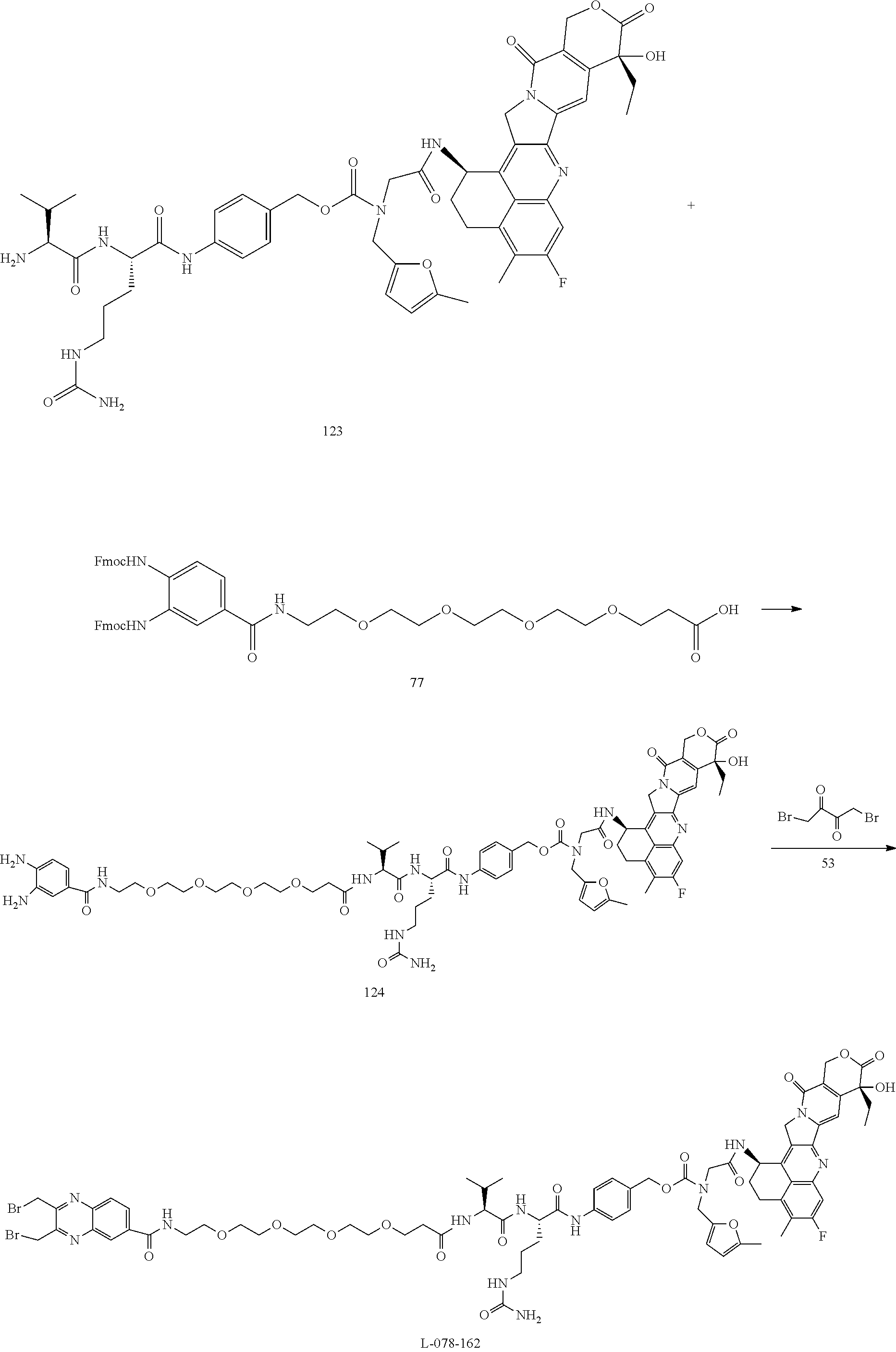

A solution of compound 75 (50 mg, 0.065 mmol), compound 121 (19 mg, 0.070 mmol), HOBT (5 mg) and DIEA (21 mg, 0.16 mmol) in DMF (3 mL) was stirred for 10 minutes to give compound 122. Compound 5 (mesylate salt, 35 mg, 0.065 mmol), HATU (25 mg, 0.065 mmol) in DMF (2 mL) and DIEA (17 mg, 0.13 mmol) were added to the solution of compound 122 (52 mg, 0.065 mmol). After being stirred for 5 minutes 0.5 ml of diethylamine was added to the solution and stirred for another 30 minutes. The mixture was purified by HPLC to give compound 123 (26 mg) as a TFA salt. To a solution of compound 123 (TFA salt, 26 mg, 0.023 mmol), compound 77 (20 mg, 0.023 mmol) and HATU (9 mg, 0.023 mmol) in DMF (3 mL) was added DIEA (8 mg, 0.059 mmol). The solution was stirred for 5 minutes followed by addition of 0.5 mL of diethylamine. The mixture was stirred for another 30 minutes, purified by HPLC, and dried to give compound 124 (18 mg). Compound 124 was dissolved in 3 ml of 60% CH₃CN/H₂O (1% TFA). To the solution of compound 124 was added a solution of compound 53 (8 mg) in CH₃CN, stirred for 5 minutes and purified by HPLC to give compound L078-171 (8 mg), MS m/z 1584.1 (M+H).

### Example S42: Synthesis of Compound L078-178

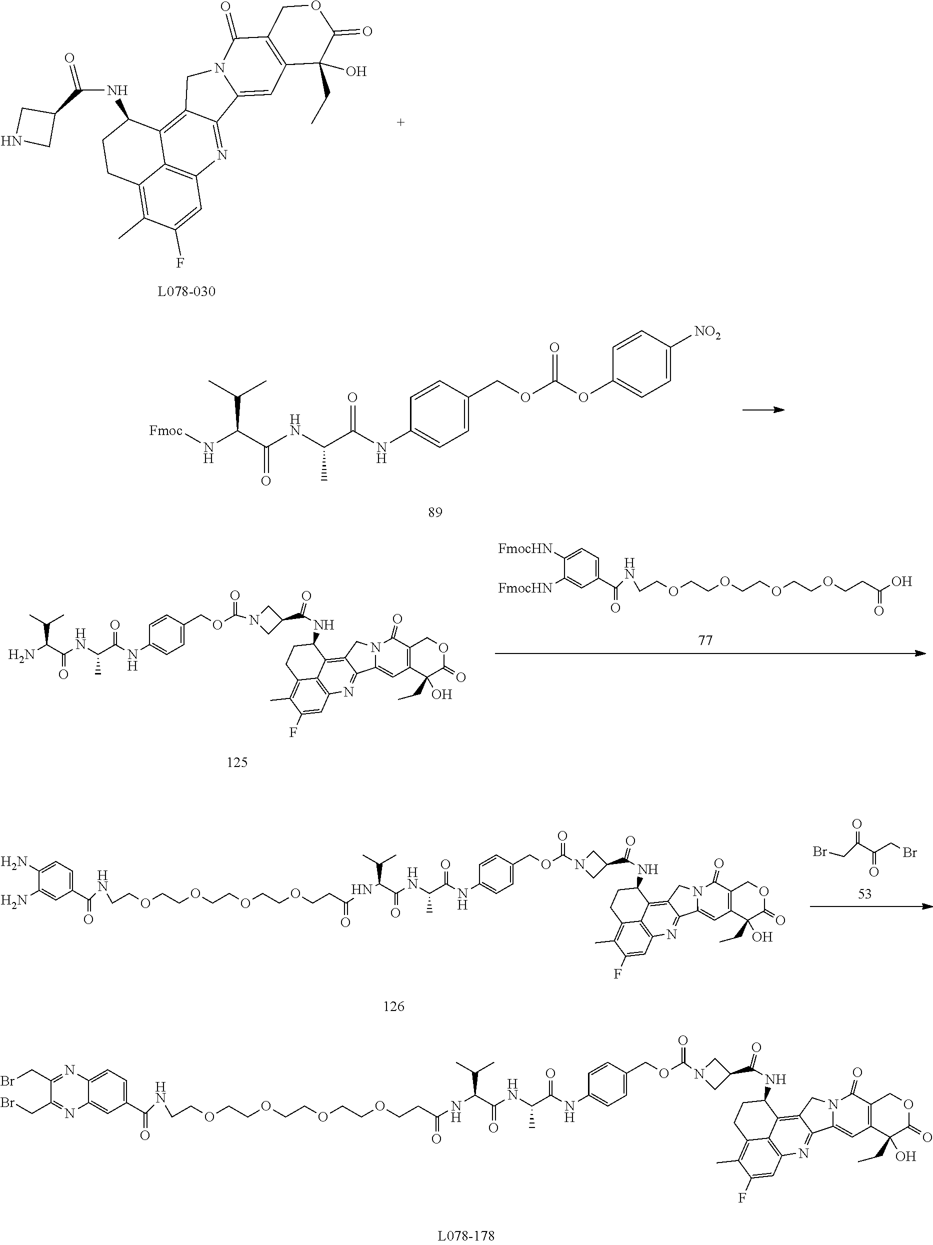

A solution of compound L078-030 (TFA salt, 30 mg, 0.047 mmol), compound 89 (39 mg, 0.057 mmol), HOBT (5 mg) and DIEA (11 mg, 0.114 mmol) in DMF (3 mL) was stirred for 30 minutes followed by addition of 0.5 ml of diethylamine and then stirred for another 30 minutes. The mixture was purified by HPLC to give compound 125 (42 mg). To a solution of compound 125 (TFA salt, 42 mg, 0.044 mmol), compound 77 (37 mg, 0.044 mmol) and HATU (17 mg, 0.044 mmol) in DMF (3 mL) was added DIEA (14 mg, 0.11 mmol). The solution was stirred for 5 minutes followed by addition of 0.5 mL of diethylamine. The mixture was stirred for additional 30 minutes, purified by HPLC and dried to give compound 126. Compound 126 was dissolved in 3 ml of 60% CH₃CN/H₂O (1% TFA). A solution of compound 53 (13 mg) in CH₃CN was added to the solution of compound 126, stirred for 5 minutes and purified by HPLC to give compound L078-178 (12 mg), m/z 1427.7 (M+H).

### Example S43: Synthesis of Compound L078-182

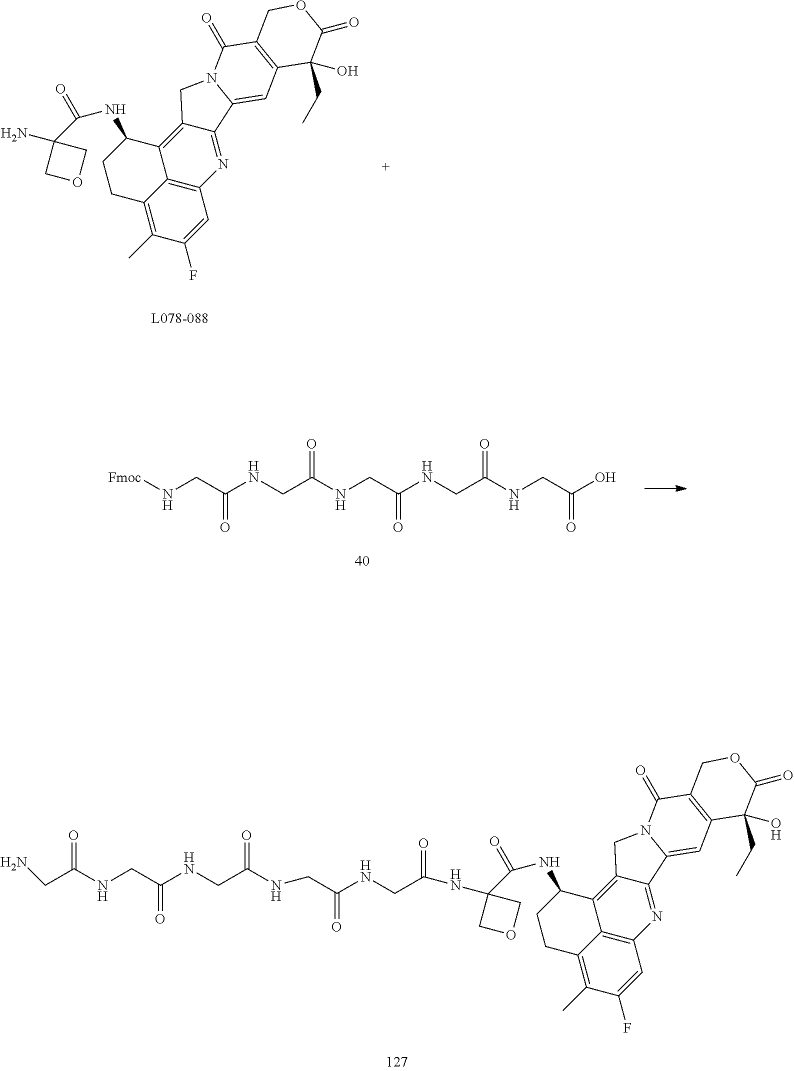

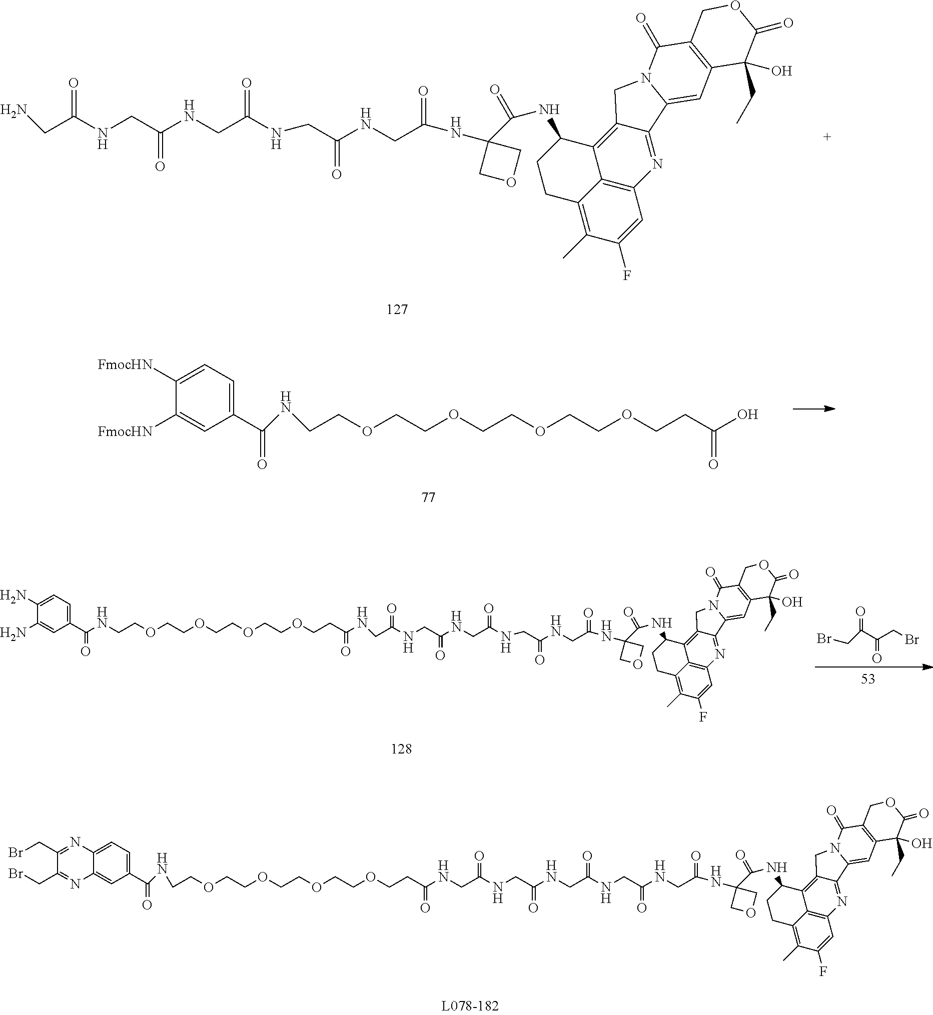

To a solution of compound L078-088 (TFA salt, 30 mg, 0.046 mmol), compound 40 (24 mg, 0.046 mmol) and HATU (18 mg, 0.046 mmol) in DMSO (3 mL) was added DIEA (11 mg, 0.114 mmol. After being stirred for 5 minutes diethylamine (0.5 mL) was added into the solution and stirred for another 30 minutes. The mixture was purified by HPLC to give compound 127 (21 mg). To a solution of compound 127 (TFA salt, 21 mg, 0.022 mmol), compound 77 (19 mg, 0.022 mmol) and HATU (9 mg, 0.022 mmol) in DMF (3 mL) was added DIEA (7 mg, 0.055 mmol). The solution was stirred for 5 minutes followed by addition of 0.5 mL of diethylamine. The mixture was stirred for 30 minutes, purified by HPLC and dried to give compound 128. Compound 128 was dissolved in 3 ml of 60% CH₃CN/H₂O (1% TFA). A solution of compound 53 (13 mg) in CH₃CN was added to the solution of compound 128, stirred for 5 minutes and purified by HPLC to give compound L078-182 (8 mg), m/z 1409.1 (M+H).

### Example S44: Synthesis of Compound L078-184

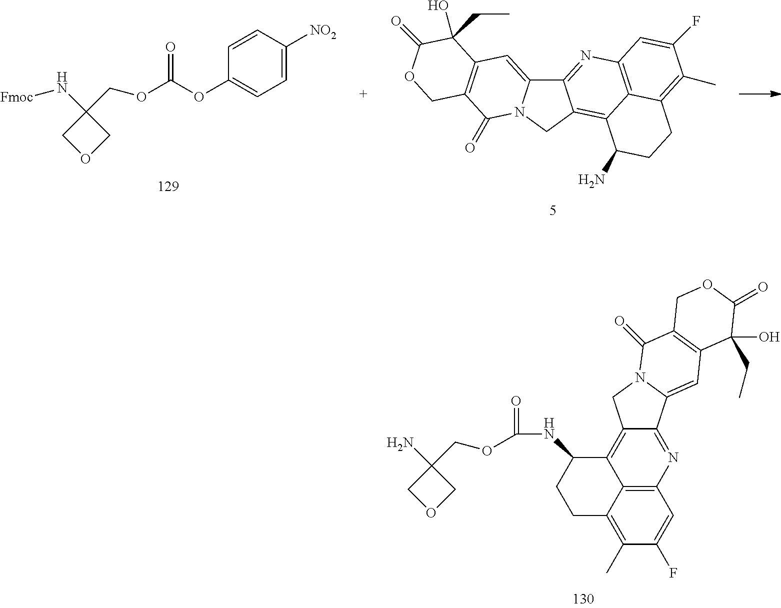

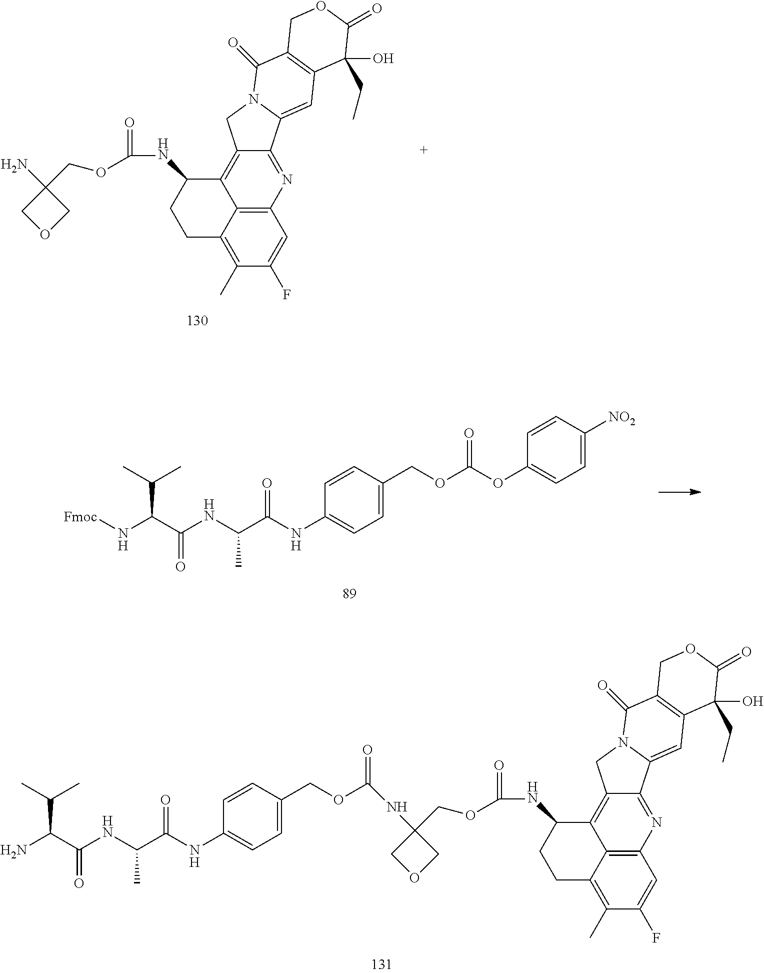

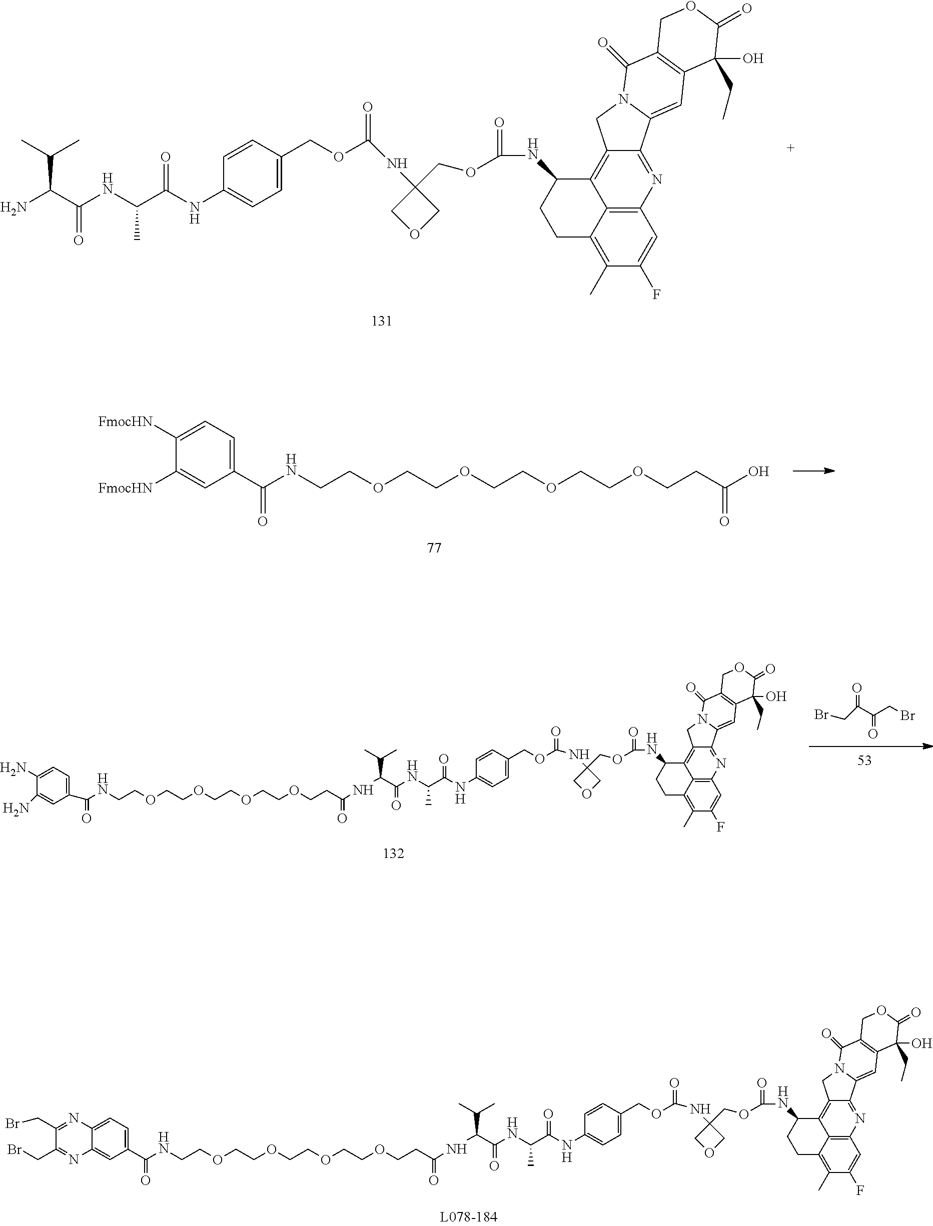

A solution of compound 129 (15 mg, 0.03 mmol), compound 5 (10 mg, 0.02 mmol), HOBT (3 mg) and DIEA (11 mg, 0.114 mmol) in DMF (3 mL) was stirred for 5 hours followed by addition of 0.5 ml of diethylamine and then stirred for another 30 minutes. The mixture was purified by HPLC to give compound 130 (21 mg). A solution of compound 130 (TFA salt, 21 mg, 0.031 mmol), compound 89 (21 mg, 0.031 mmol), HOBT (5 mg) and DIEA (10 mg) in DMF (3 mL) was stirred for 30 minutes followed by addition of 0.5 mL of diethylamine. The mixture was stirred for another 30 minutes, purified by HPLC, and dried to give compound 131 (16 mg). To a solution of compound 131 (16 mg, 0.016 mmol), compound 77 (14 mg, 0.016 mmol) and HATU (6 mg, 0.016 mmol) in DMF (2 mL) was added DIEA (5 mg, 0.04 mmol). The solution was stirred for 5 minutes followed by addition of 0.5 mL of diethylamine and then stirred for additional 30 minutes. The solution was purified by HPLC and dried to give compound 132. Compound 132 was dissolved in 3 ml of 60% CH₃CN/H₂O (1% TFA). Solution of compound 53 (5 mg) in CH₃CN was added to the solution of compound 132. The solution was purified by HPLC to give compound L078-184 (4 mg), m/z 1475.3 (M+H).

### Example S45: Synthesis of Compound L081-034

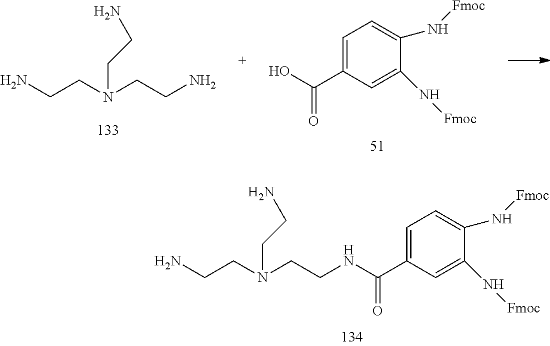

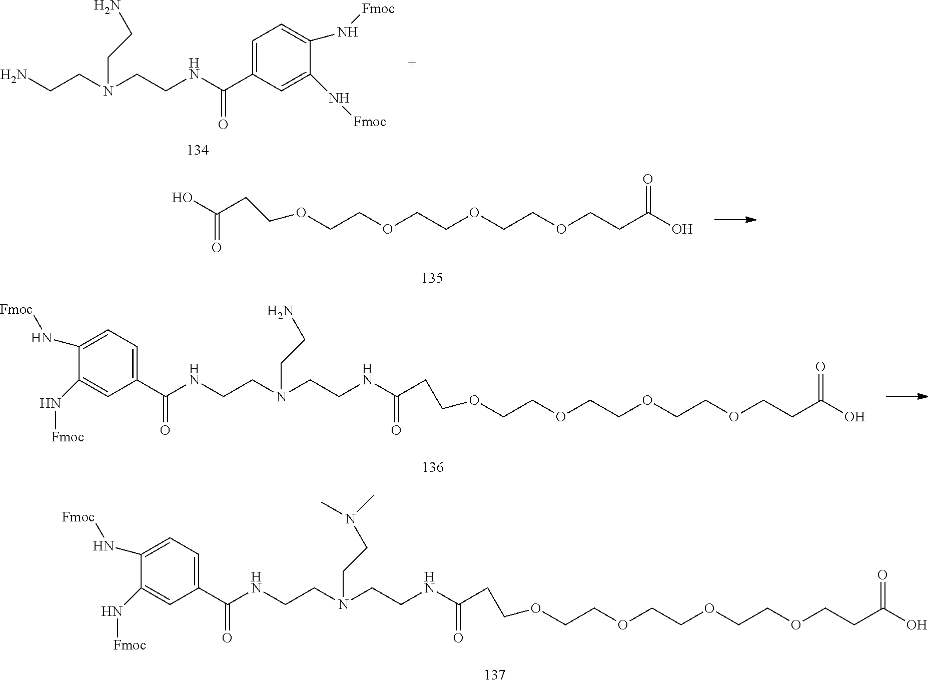

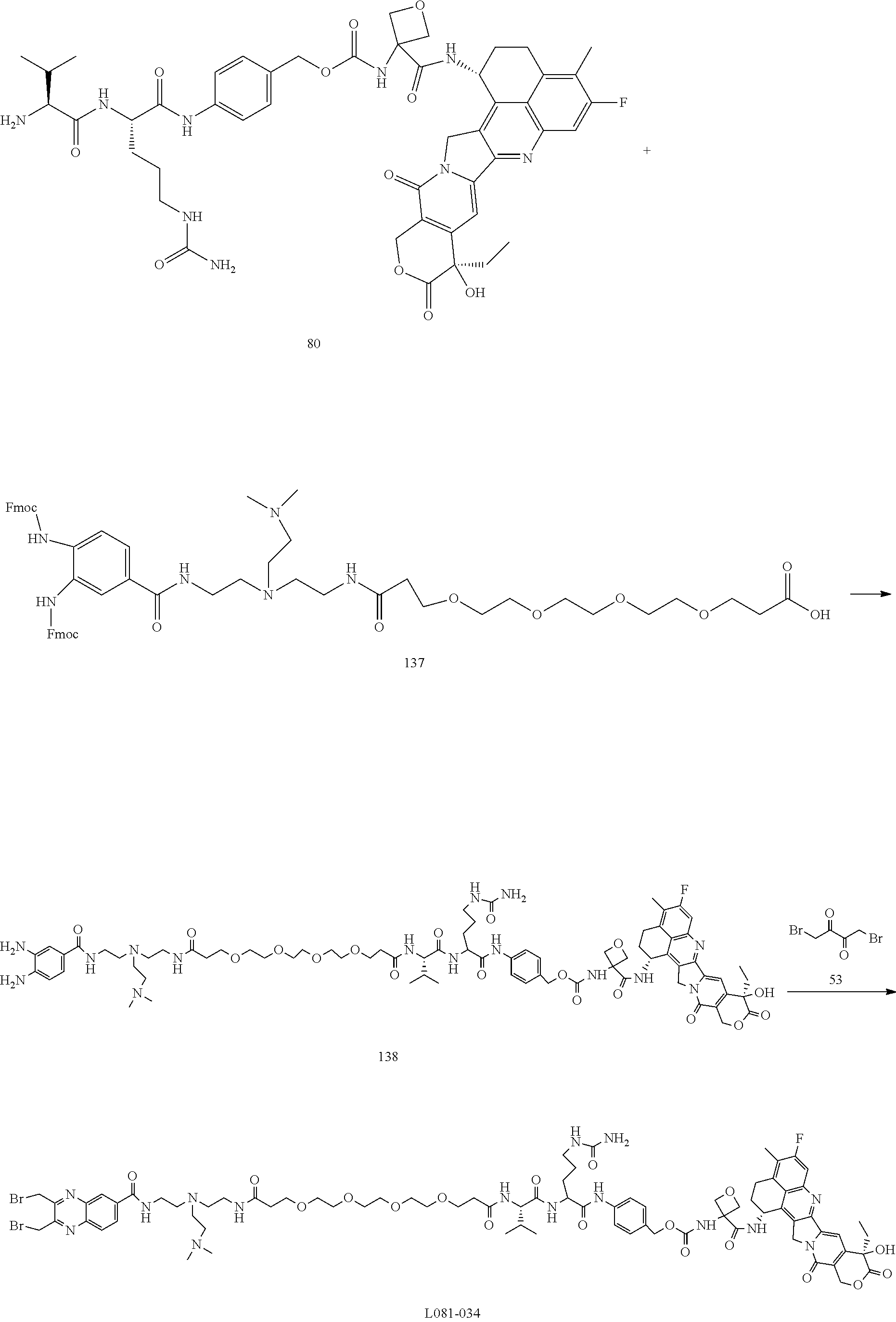

A solution of compound 51 (500 mg, 0.839 mmol), HATU (319 mg, 0.839 mmol) and DIEA (108 mg, 0.839 mmol) in DMF (3 mL) was stirred for 1 minute. The solution was then added to a solution of compound 133 (CAS #33527-91-2; 367 mg, 2.52 mmol) in DCM (20 mL) dropwise. DCM was evaporated under reduced pressure. The residue was purified by HPLC to give compound 134 (172 mg).

A solution of compound 134 (TFA salt, 60 mg, 0.057 mmol), compound 135 (17 mg, 0.057 mmol), HATU (22 mg, 0.057 mmol) and DIEA (29 mg, 0.228 mmol) in DMF (3 mL) was stirred for 5 minutes. The mixture was purified by HPLC and concentrated to give compound 136. To a solution of compound 136 (crude in situ) in MeOH (2 ml) was added formaldehyde (37% in water, 0.2 ml). The solution was stirred for 5 minutes followed by addition of NaCNBH₃ (10 mg) and then stirred for additional 30 minutes. The solution was purified by HPLC to give compound 137 (24 mg). A solution of compound 137 (TFA salt, 24 mg, 0.023 mmol), compound 80 (TFA salt, 25 mg, 0.023 mmol), HATU (9 mg, 0.023 mmol) and DIEA (12 mg, 0.092 mmol) in DMF (2 ml) was stirred for 5 minutes followed by addition of diethylamine (0.5 ml), stirred for another 30 minutes and purified by HPLC to give compound 138. Compound 138 was dissolved in 60% CH₃CN/H₂O (0.5% TFA) and mixed with a solution of compound 53 (5 mg) in acetonitrile. The solution was purified by HPLC to give compound L081-034 (9 mg), m/z 1713.9 (M+H).

### Example S46: Synthesis of Compound L081-036

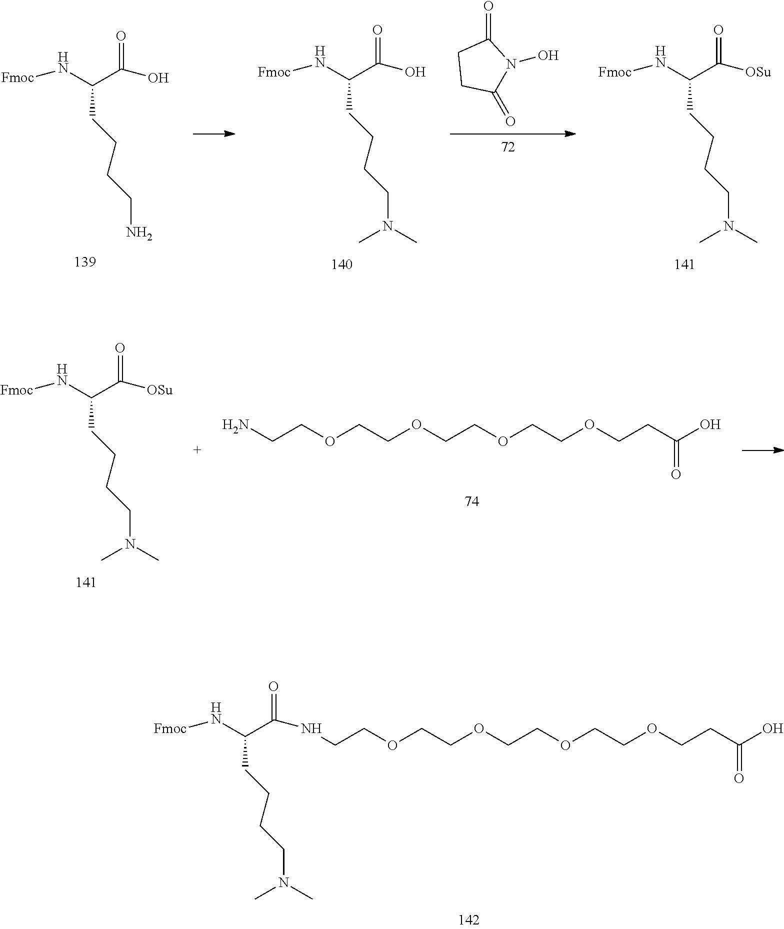

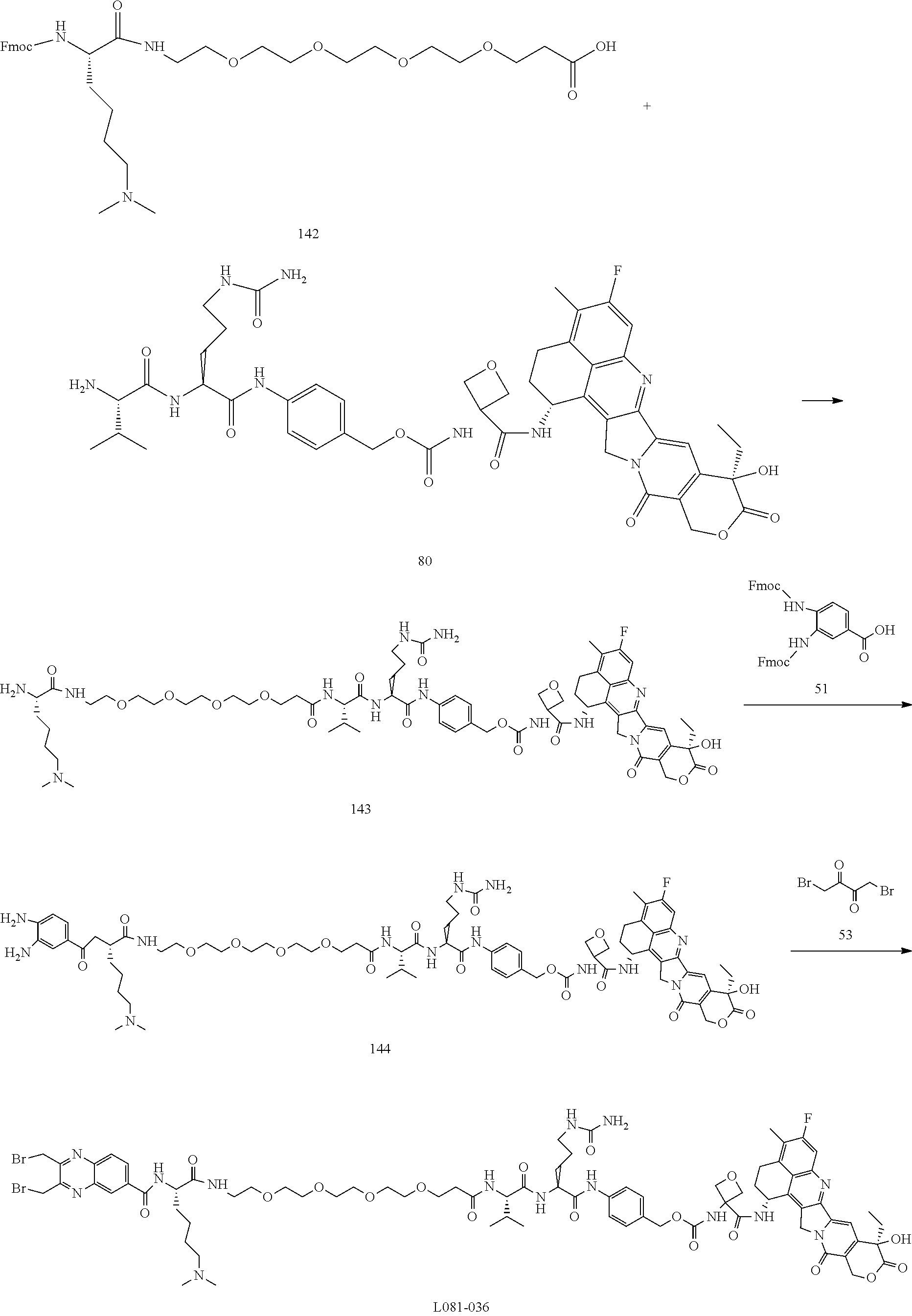

To a solution of compound 139 (CAS #139262-23-0; hydrochloride salt, 500 mg, 1.237 mmol) in MeOH (10 mL) was added 37% CH₂O (1 mL). The solution was stirred for 2 minutes followed by addition of NaCNBH₃ (200 mg). The solution was stirred for 20 minutes and purified by HPLC to give compound 140 (502 mg). Solution of compound 140 (150 mg, 0.294 mmol), compound 72 (CAS #6066-82-6; 50 mg, 0.44 mmol) and EDC-HCl (168 mg, 0.882 mmol) in DCM was stirred for 1 hour. The solution was extracted with EtOAc and concentrated, yielding compound 141. Compound 141 was dissolved in 50% CH₃CN/H₂O (5 mL). To the solution was added compound 74 (CAS #663921-15-1; 78 mg, 0.294 mmol) and 10% NaHCO₃ (bring solution to pH 8.5). The solution was stirred for 30 minutes, concentrated, and purified to give compound 142 (157 mg).

A solution of compound 142 (15 mg, 0.02 mmol), compound 80 (20 mg, 0.02 mmol), HATU (8 mg, 0.02 mmol) and DIEA (10 mg, 0.08 mmol) in DMF (3 mL) was stirred for 5 minutes. The mixture was purified by HPLC and concentrated to give compound 143 (17 mg). A solution of 6 compound 143 (17 mg, 0.011 mmol), compound 51 (7 mg, 0.011 mmol), HATU (5 mg, 0.011 mmol) and DIEA (6 mg, 0.044 mmol) was stirred in DMF (2 ml) for 5 minutes followed by addition of diethylamine (0.5 ml), and then stirred for additional 30 minutes. The solution was purified by HPLC to give compound 144. Compound 144 was mixed with a solution of compound 53 (5 mg) in acetonitrile. The solution was purified by HPLC to give compound L081-036 (12 mg), m/z 1686.2 (M+H).

### Example S47: Synthesis of Compound L081-038

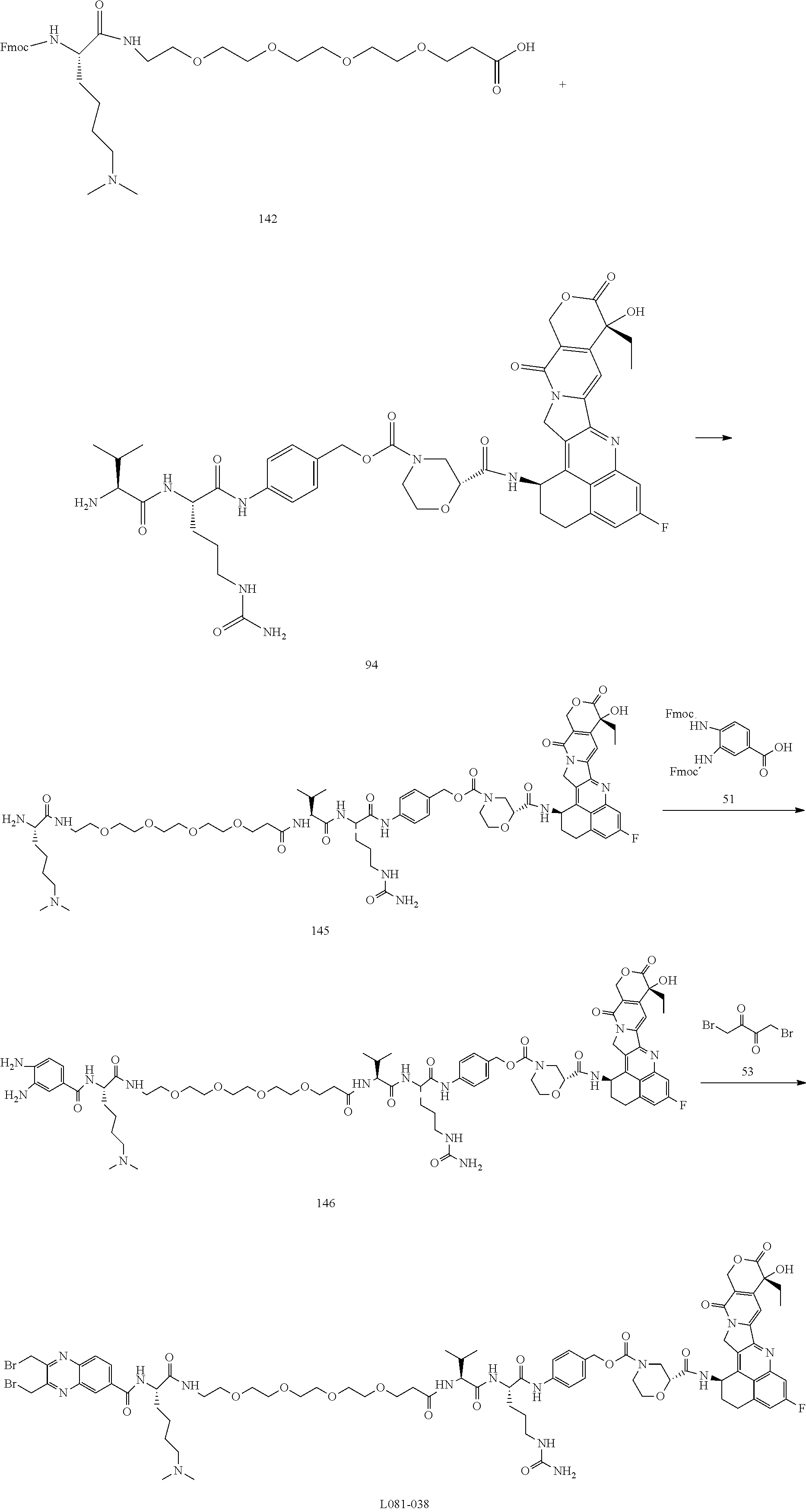

A solution of compound 142 (TFA salt, 15 mg, 0.023 mmol), compound 94 (TFA salt, 20 mg, 0.023 mmol), HATU (8 mg, 0.02 mmol) and DIEA (10 mg, 0.08 mmol) in DMF (3 mL) was stirred for 5 minutes. The mixture was purified by HPLC and concentrated to yield compound 145 (20 mg). A solution of compound 145 (TFA salt, 20 mg, 0.013 mmol), compound 51 (8 mg, 0.013 mmol), HATU (5 mg, 0.013 mmol) and DIEA (7 mg, 0.052 mmol) was stirred in DMF (2 ml) for 5 minutes followed by addition of diethylamine (0.5 ml), and then stirred for another 30 minutes. The solution was purified by HPLC to give compound 146. Compound 146 was mixed with a solution of compound 53 (5 mg) in acetonitrile. The solution was purified by HPLC to give compound L081-038 (13 mg), m/z 1701.2 (M+H).

### Example S48: Synthesis of Compound L081-045

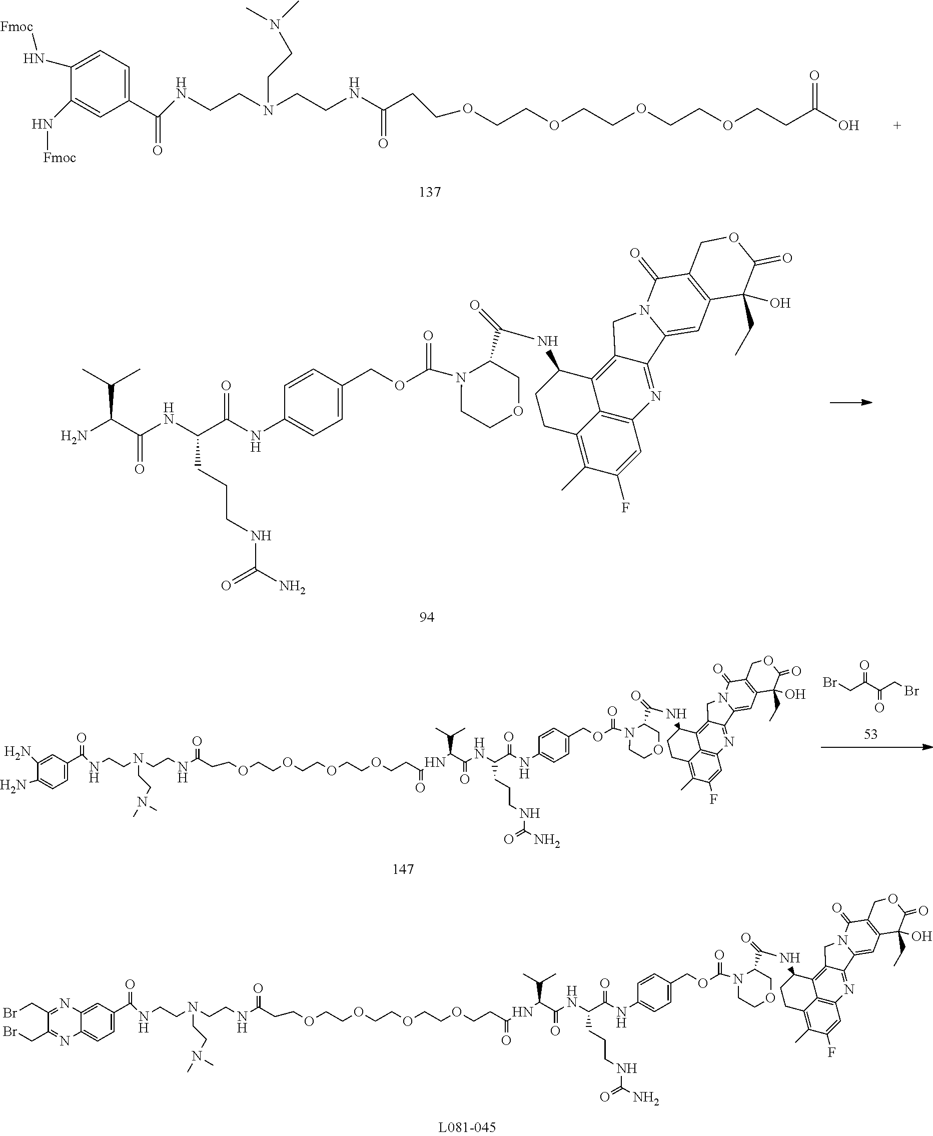

A solution of compound 137 (TFA salt, 18 mg, 0.014 mmol), compound 94 (15 mg, 0.014 mmol), HATU (6 mg, 0.014 mmol) and DIEA (8 mg, 0.056 mmol) was stirred in DMF (2 ml) for 5 minutes followed by addition of diethylamine (0.5 ml), and then stirred for another 30 minutes and purified by HPLC to give compound 147. Compound 147 was dissolved in 60% CH₃CN/H₂O (0.5% TFA) and mixed with a solution of compound 53 (5 mg) in acetonitrile. The solution was purified by HPLC to give compound L081-045 (5 mg), m/z 1728.6 (M+H).

### Preparation of Antibody-Drug Conjugates (ADCs)

Antibody-Drug Conjugates (ADCs) were prepared by conjugating a drug-linker compound, whose synthesis is provided above, with anti-HER2 antibody.

Three generally applicable procedures for conjugating the drug-linker compound to an antibody, such as an anti-HER2 antibody, were developed. The procedure described for preparation of ADC-1 is used for conjugation of compounds comprising 2,3-bis(bromomethyl)quinoxaline to thiols of cysteine group(s). The procedure described for preparation of ADC-2 is used for conjugation of compounds comprising maleimide to thiols of cysteine group(s). The procedure described for preparation of ADC-3 is used for conjugation of compounds with activated carboxylic acids to the amine of lysine groups.

### Preparation of ADC-1

Affinity purified anti-HER2 antibody was buffer exchanged into Conjugation Buffer (50 mM sodium phosphate buffer, pH 7.2, 4 mM EDTA) at a concentration of 5 mg/mL. To a portion of this antibody stock was added a freshly prepared 10 mM water solution of tris(2-carboxyethyl)phosphine) (TCEP) at 15-fold molar excess. The resulting mixture was incubated at 4-8° C. overnight. The excess unreacted TCEP was then removed by several rounds of centrifugal ultrafiltration with fresh Conjugation Buffer. Recovery of reduced antibody material was quantified via UV-Vis analysis relative to the antibody extinction coefficient.

To initiate conjugation of drug-linker compound to antibody, the compound was freshly dissolved in a 3:2 acetonitrile/water mixture to a concentration of 5 mM. Propylene glycol (PG) was then added to a portion of the reduced, purified (TCEP removed) anti-HER2 antibody to give a final concentration of 20% (v/v) PG immediately prior to addition of drug-linker compound in 8-fold molar excess. After thorough mixing and incubation at ambient temperature for 2 hr, the crude conjugation reaction was analyzed by HIC-HPLC chromatography to confirm reaction completion (disappearance of starting antibody peak) at 280 nm wavelength detection. Purification of the resulting ADC-conjugate (ADC-1) was then carried out by gel-filtration chromatography using an AKTA system equipped with a Superdex 200 pg column (GE Healthcare) equilibrated with PBS. The average drug-to-antibody ratio (DAR) was calculated based on comparative peak area integration of the HIC-HPLC chromatogram. Confirmation of low percent (<5%) high molecular weight (HMW) aggregates for the resulting ADC-conjugate (ADC-1) was determined using analytical SEC-HPLC.

### Preparation of ADC-2

Affinity purified anti-HER2 antibody was buffer exchanged into Conjugation Buffer in a manner identical to ADC-1. To a portion of this anti-HER2 antibody solution was added a freshly prepared 10 mM water solution of TCEP at 3-fold molar excess. The resulting mixture was incubated at 37° C. for 2 hr. Drug-linker compound was then freshly dissolved in anhydrous dimethylsulfoxide (DMSO) to 5 mM. A portion of this mixture was added to the reduced anti-HER2 antibody solution in 6-fold molar excess. After thorough mixing and incubation at ambient temperature for 2 hr, the crude conjugation reaction was analyzed by HIC-HPLC chromatography to confirm reaction completion (disappearance of starting antibody peak) at 280 nm wavelength detection. Purification and analysis of the resulting ADC-conjugate (ADC-2) proceeded in a manner identical to ADC-1. The resulting average DAR for was calculated based on comparative peak area integration of the HIC-HPLC chromatogram. Confirmation of low percent (<5%) high molecular weight (HMW) aggregates for the resulting ADC-conjugate (ADC-2) was determined using analytical SEC-HPLC.

### Preparation of ADC-3

To a solution of 0.5-50 mgs/mL of antibody in buffer at pH 6.0-9.0 with 0-30% organic solvent, was added 0.1-10 eq of activated drug linker conjugate in a manner of portion wise or continuous flow. The reaction was performed at 0-40° C. for 0.5-50 hours with gentle stirring or shaking, monitored by HIC-HPLC. The resultant crude ADC product underwent necessary down-stream steps of desalt, buffet changes/formulation, and optionally, purification, using the state-of-art procedures in a manner identical to ADC-1. The ADC product was characterized by HIC-HPLC. The resulting average DAR for was calculated based on comparative peak area integration of the HIC-HPLC chromatogram.

### Example B1: In Vitro Efficacy of Camptothecin Derivatives

The in vitro efficacies of camptothecin derivatives were evaluated using the following human cancer cell lines: SkBr-3 (Her2+) and MDA-MB-468 (HER2−), purchased from the American Type Culture Collection (ATCC; Manassas, VA) and were routinely cultured in DMEM/F-12 medium (Catalog #10-090-CV; Corning) supplemented with 10% fetal bovine serum (FBS; Catalog #MT35011CV; Corning) and 1× Penicillin-Streptomycin (Catalog #30-002-CI; Corning) and maintained at 37° C. with 5% CO2 in a humidified environment.

Tumor cells were harvested by detachment with cell stripper. Viable cell counts were made by Trypan blue exclusion using a Countess or Countess II automated cell counter. Cell Viability Assay: All cells were harvested and seeded into 384-well white wall clear bottom plates (Catalog #3765; Corning) at a density of 875 cells/well in DMEM/F-12 medium supplemented with 10% fetal bovine serum and 1× Penicillin-Streptomycin (complete growth media). Plates were maintained at 37° C. for 4-6 hours to allow cells to adhere to the plate. The outer wells of plates contained complete growth medium only and were used for background subtraction for the cell viability assay. Working solutions of test articles were prepared at 2× final concentrations with 5-fold serial dilutions in complete growth medium. Cell treatment was performed in triplicates and maintained at 37° C. for 120-hour assay. After treatment, cell viability was determined by CellTiter-Glo 2.0 assay (Catalog #G9243; Promega; Madison, WI, USA) based on the manufacturer's instructions. CellTiter Glo reagent reacts with ATP in metabolically active cells to give a luminescent readout that is directly proportional to the number of viable cells. Briefly, plates were removed from the incubator and equilibrated to room temperature before addition of CellTiter Glo reagent. Luminescence was measured using a Tecan Spark microplate reader (Tecan; Mannedorf, Switzerland).

For Cytotoxicity assays, raw luminescence data was background subtracted with average luminescence from the outer wells containing medium only and normalized to untreated controls using Excel (Microsoft; Albuquerque, NM). Dose-response relationships and EC50 values were determined based on non-linear regression analysis of normalized data fit to a four-parameter logistic equation using GraphPad Prism 8.0.

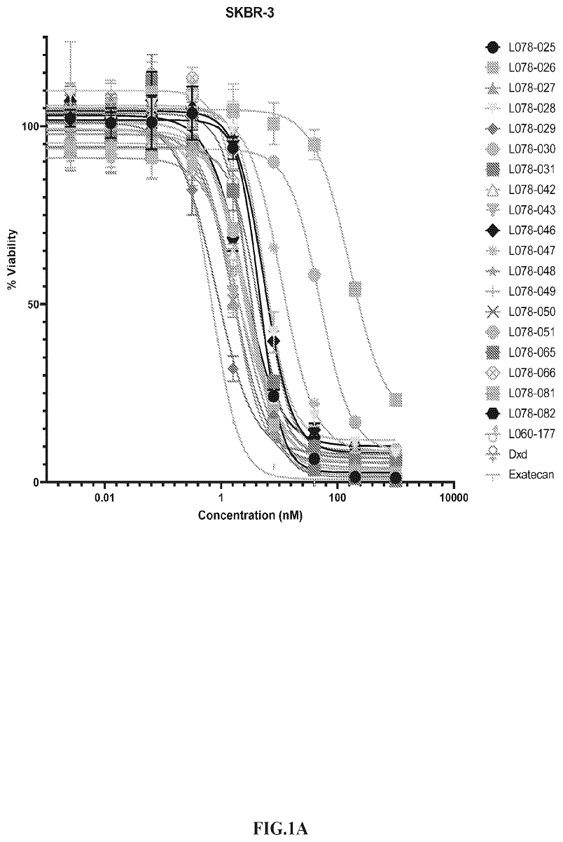

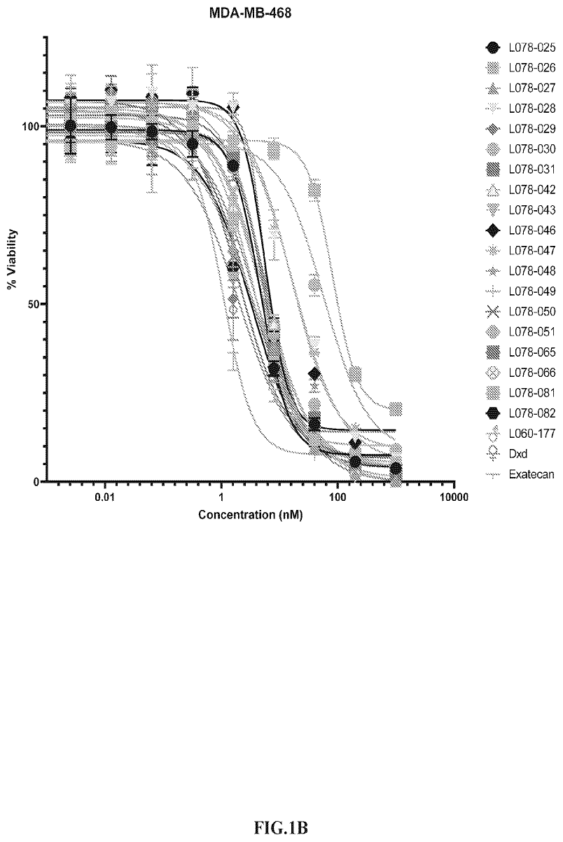

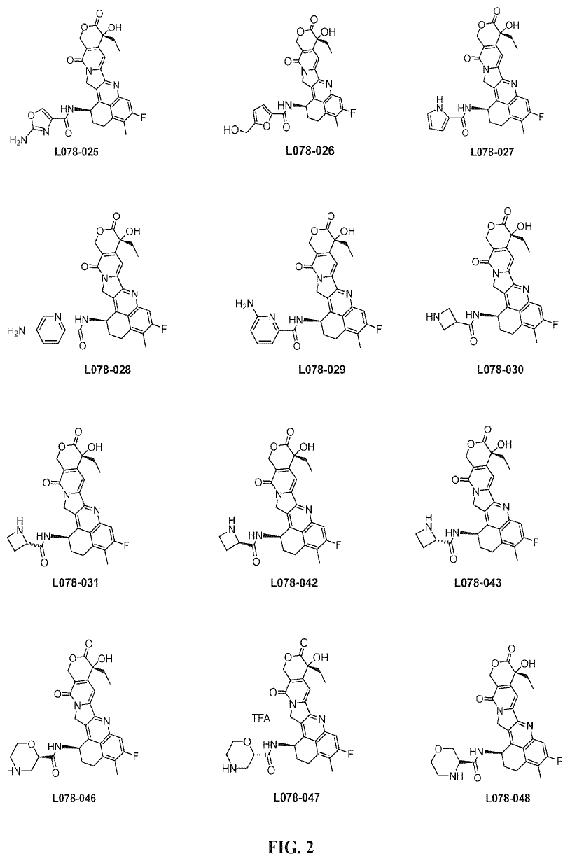

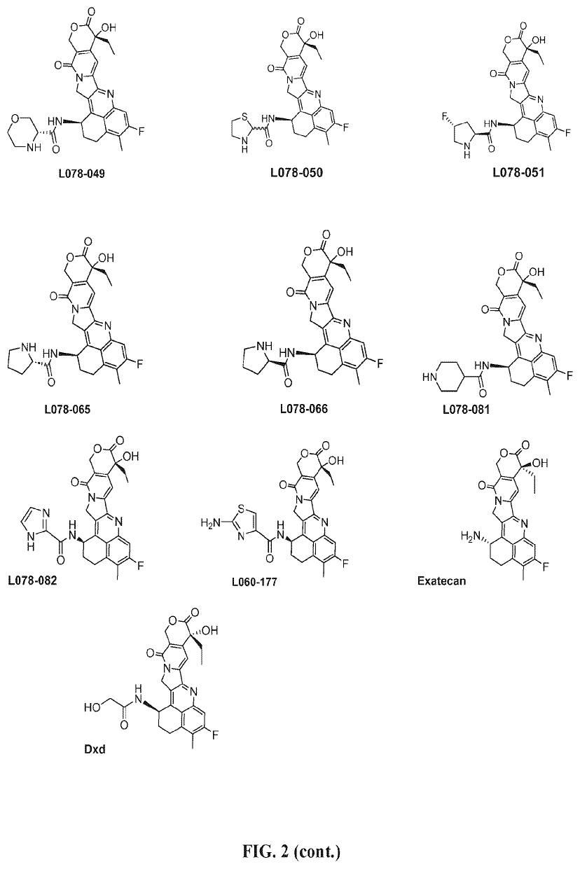

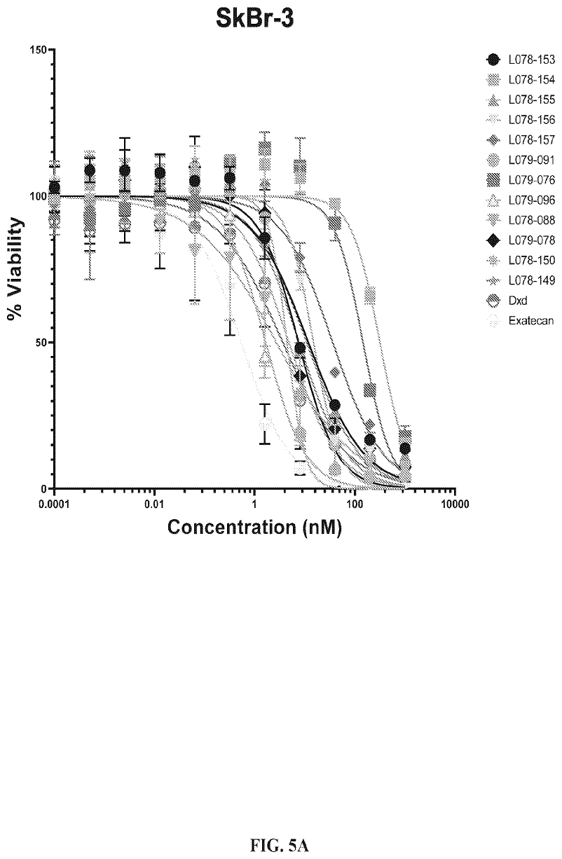

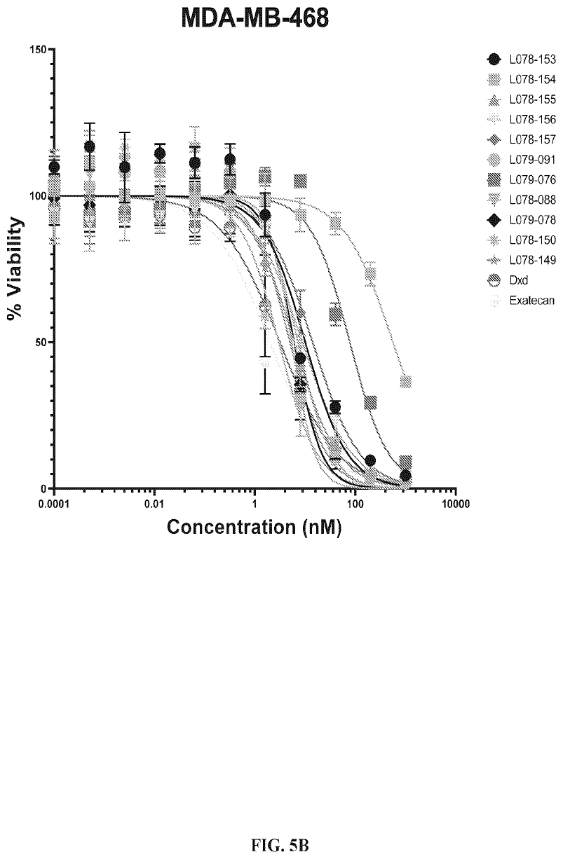

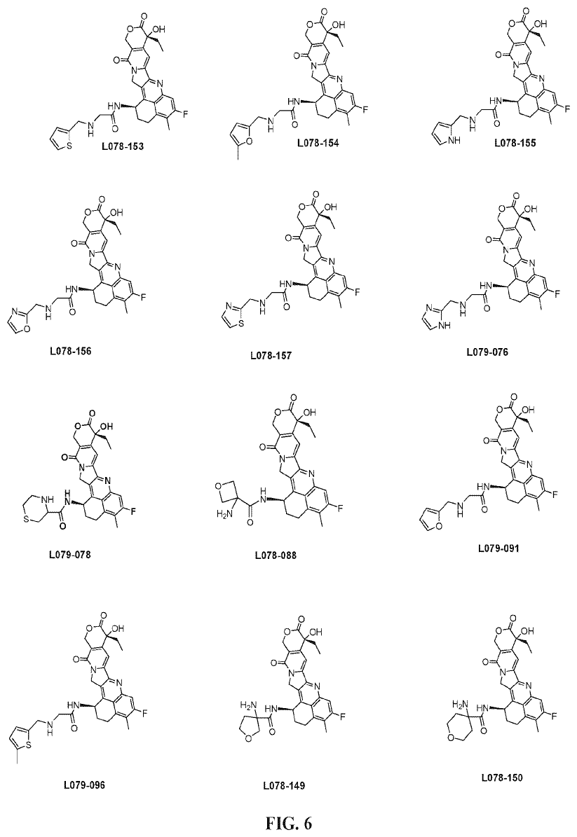

Cell viability, for camptothecin derivatives is shown in FIGS. 1A, 1B, 5A, and 5B, and Tables 4A and 4B. Corresponding structures of the camptothecin derivatives are shown in FIGS. 2 and 6.

In vitro cytotoxic activities of the camptothecin derivatives (TFA salt form) described herein (and controls: exatecan mesylate salt and Dxd TFA salt) were evaluated against HER2-expressing SkBr-3 and HER2-negative MDA-MB-468 cancer cell lines using standard cell viability assays. As shown in FIGS. 1A, 1B, 5A, and 5B, camptothecin derivatives dose-dependently reduced SkBr-3 and MDA-MB-468 cell viability in 5-day assays. A range in potency as determined by EC50 was determined to be -0.7 to 564 nM, although most EC50s were in the single digit range (Tables 4A and 4B). Camptothecin derivatives inhibited cell proliferation across both cell lines in a dose-dependent manner regardless of HER2 expression level.

Summary of EC50 Values (nM) of camptothecin derivatives in Human Tumor Cells is presented in Tables 4A and 4B.

TABLE 4A
EC₅₀ Values (nM) of camptothecin
derivatives in Human Tumor Cells

| | |
|---|---|
| Derivative | EC50 (nM) |

| number | SkBr-3 (+) | MDA-MB-468 (−) |
|---|---|---|
| L078-025 | 4.6 | 4.7 |
| L078-026 | 2.8 | 3.6 |
| L078-027 | 1.6 | 4.2 |
| L078-028 | 5.6 | 16.8 |
| L078-029 | 0.8 | 2.0 |
| L078-030 | 49.6 | 57.2 |
| L078-031 | 4.3 | 5.7 |
| L078-042 | 2.3 | 5.7 |
| L078-043 | 1.8 | 3.7 |
| L078-046 | 5.5 | 5.7 |
| L078-047 | 11.0 | 15.7 |
| L078-048 | 5.6 | 5.7 |
| L078-049 | 2.3 | 4.7 |
| L078-050 | 1.6 | 3.0 |
| L078-051 | 2.8 | 5.4 |
| L078-065 | 3.2 | 6.0 |
| L078-066 | 3.0 | 5.6 |
| L078-081 | 162.7 | 81.4 |
| L078-082 | 2.5 | 3.2 |
| L060-177 | 2.7 | 5.1 |
| Exatecan | 0.69 | 1.1 |
| Dxd | 2.2 | 2.0 |

TABLE 4B
EC₅₀ Values (nM) of camptothecin derivatives
in Human Tumor Cells (nM)

| | |
|---|---|
| Derivative | EC50 (nM) |

| number | SkBr-3 (+) | MDA-MB-468 (−) |
|---|---|---|
| L078-153 | 11.82 | 10.08 |
| L078-154 | 316.70 | 564.20 |
| L078-155 | 10.95 | 6.07 |
| L078-156 | 14.93 | 8.83 |
| L078-157 | 36.22 | 14.13 |
| L079-091 | 4.44 | 5.44 |
| L079-076 | 145.80 | 77.85 |
| L079-096 | 1.67 | N/D |
| L078-088 | 2.72 | 7.32 |
| L079-078 | 7.52 | 6.05 |
| L078-150 | 4.83 | 3.01 |
| L078-149 | 7.71 | 5.01 |
| Dxd | 3.66 | 3.16 |
| Exatecan | 0.54 | 1.99 |

### Example B2: In Vitro Efficacy of Antibody-Drug Conjugates (ADCs)

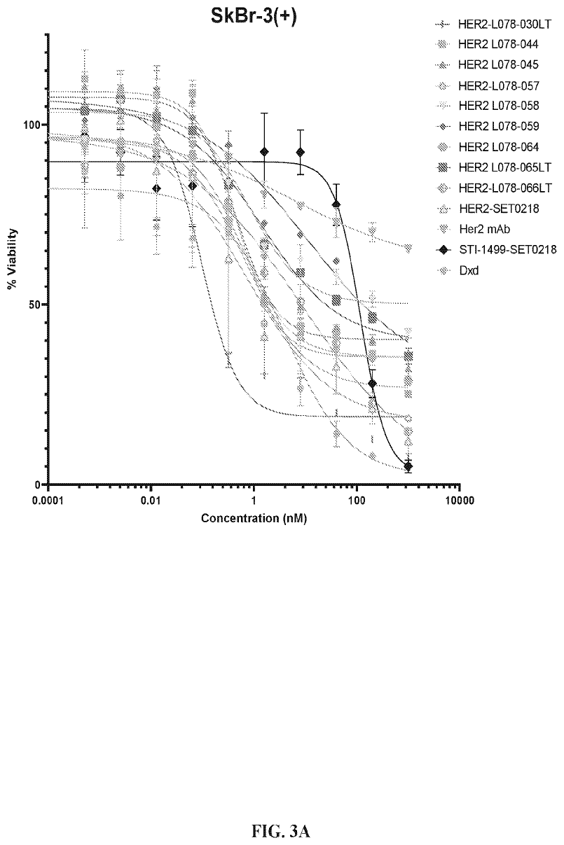

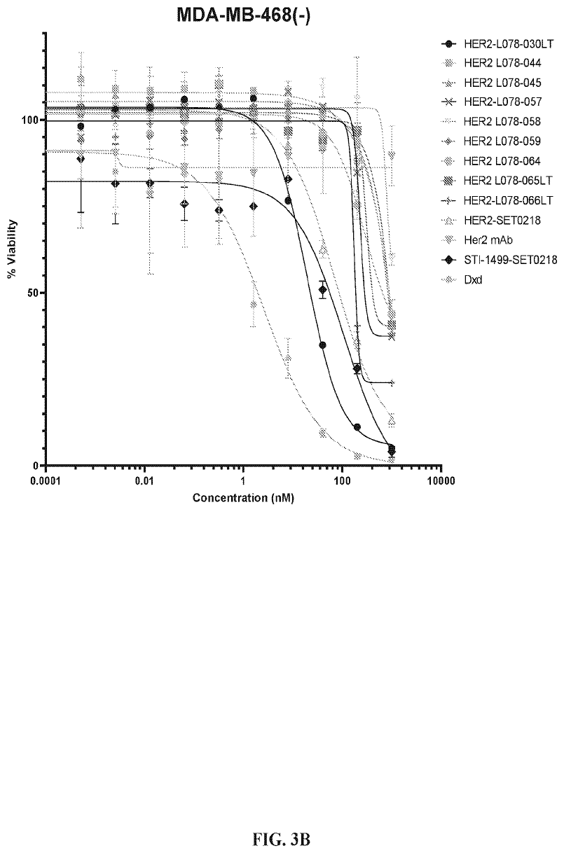

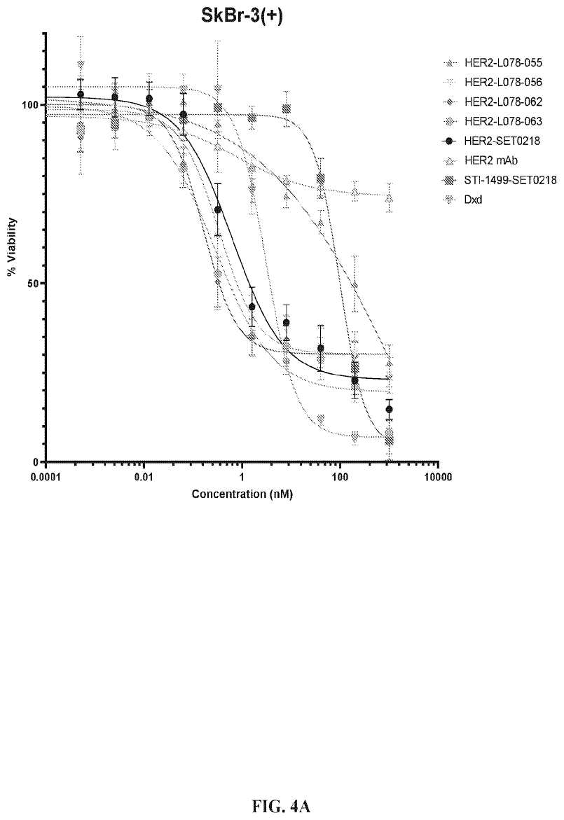

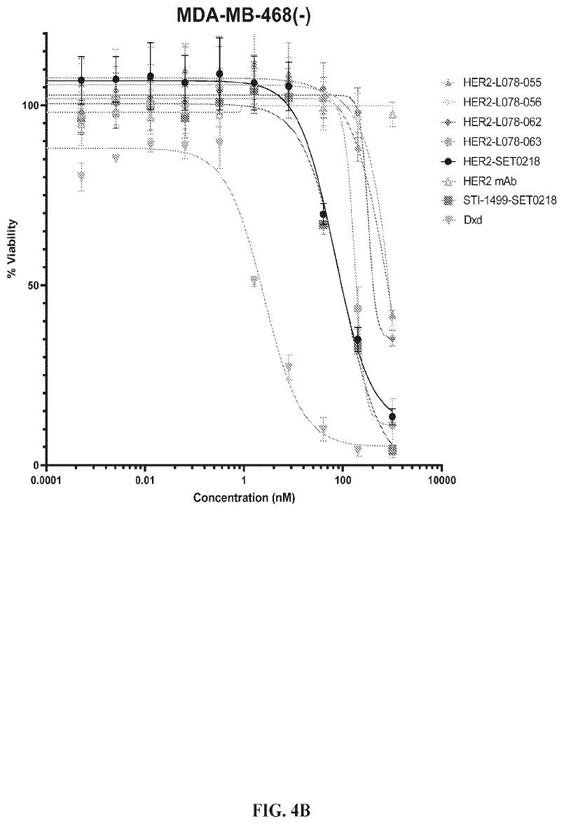

The in vitro efficacies of our ADCs were compared with efficacy of anti-HER2-Dxd also labeled as HER2-SET0218 in FIGS. 3A, 3B, 4A, and 4B (where Dxd is covalently bound to anti-HER2 antibody via maleimide-glycine-glycine-phenylalanine-glycine (GGFG) peptide linker). Anti-HER2 antibody was conjugated to compounds L078-030-LT, L078-044, L078-045, L078-055, L078-056, L078-057, L078-058, L078-059, L078-062, L078-063, L078-064, L078-065LT, L078-066LT, L079-018, L079-019, L079-027, L079-034, L079-035, L079-040, L078-121, L078-118, L078-119, L078-120, L078-177, L078-130, L078-123, L078-163, L078-164, L078-173, L078-170, L078-171, L078-178, L078-182, L081-034, L081-036, or L081-038. The anti-HER2 antibody comprised the VL sequence of SEQ ID NO: 7 and the VH sequence of SEQ ID NO: 8.

The resulting average drug-antibody-ratio (DAR) for ADC-L079-040 was -1.0. The resulting average DAR for ADC-L079-018 and ADC-L079-019 was 1.8-1.9. The resulting average DAR for ADC-L079-034 and ADC-L079-027 was 2.2-2.3. The resulting average DAR for ADC-L078-164, ADC-L078-171, and ADC-L078-123 was 2.75-3.05. The resulting average DAR for ADC-L078-178, ADC-L078-170, ADC-L078-177, ADC-L078-120, ADC-L078-118, ADC-L079-034, ADC-L079-036, ADC-L078-121, ADC-L078-163, and ADC-L078-173 was 3.2-3.5. The resulting average DAR for ADC-L078-044, ADC-L078-045, ADC-L078-058, ADC-L078-059, ADC-L078-063, ADC-L078-064, ADC-L078-066LT, ADC-L078-182, ADC-L078-130, ADC-L079-035, and control ADC-SET-0218(DAR4) was 3.8-4.1. The resulting average DAR for ADC-L078-056, ADC-L078-057, ADC-L078-119, ADC-L081-038, and ADC-L078-062 was 3.6-3.7. The resulting average DAR for ADC-L078-065LT was 4.4. The resulting average DAR for ADC-L078-030-LT and ADC-SET0218(DAR8) was 6.2-6.4.

ADCs were evaluated using the following human cancer cell lines: HER2-positive SkBr-3, HER2-positive NCI-N87, and HER2-negative MDA-MB-468, purchased from the American Type Culture Collection (ATCC; Manassas, VA) and were routinely cultured in DMEM/F-12 medium (Catalog #10⁻⁰⁹⁰-CV; Corning) supplemented with 10% fetal bovine serum (FBS; Catalog #MT35011CV; Corning) and 1× Penicillin-Streptomycin (Catalog #30-002-CI; Corning) and maintained at 37° C. with 5% CO2 in a humidified environment.

Tumor cells were harvested by detachment with cell stripper. Viable cell counts were made by Trypan blue exclusion using a Countess or Countess II automated cell counter. Cell Viability Assay: All cells were harvested and seeded into 384-well white wall clear bottom plates (Catalog #3765; Corning) at a density of 875 cells/well in DMEM/F-12 medium supplemented with 10% fetal bovine serum and 1× Penicillin-Streptomycin (complete growth media). Plates were maintained at 37° C. for 4-6 hours to allow cells to adhere to the plate. The outer wells of plates contained complete growth medium only and were used for background subtraction for the cell viability assay. Working solutions of test articles were prepared at 2× final concentrations with 5-fold serial dilutions in complete growth medium. Cell treatment was performed in triplicates and maintained at 37° C. for 120-hour assay. After treatment, cell viability was determined by CellTiter-Glo 2.0 assay (Catalog #G9243; Promega; Madison, WI, USA) based on the manufacturer's instructions. CellTiter Glo reagent reacts with ATP in metabolically active cells to give a luminescent readout that is directly proportional to the number of viable cells. Briefly, plates were removed from the incubator and equilibrated to room temperature before addition of CellTiter Glo reagent. Luminescence was measured using a Tecan Spark microplate reader (Tecan; Mannedorf, Switzerland).

For Cytotoxicity assays, raw luminescence data was background subtracted with average luminescence from the outer wells containing medium only and normalized to untreated controls using Excel (Microsoft; Albuquerque, NM). Dose-response relationships and EC50 values were determined based on non-linear regression analysis of normalized data fit to a four-parameter logistic equation using GraphPad Prism 8.0.

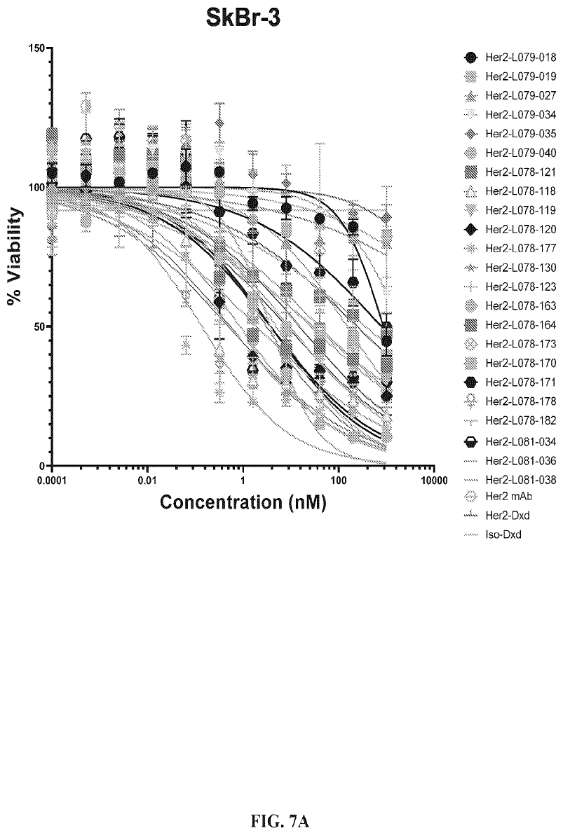

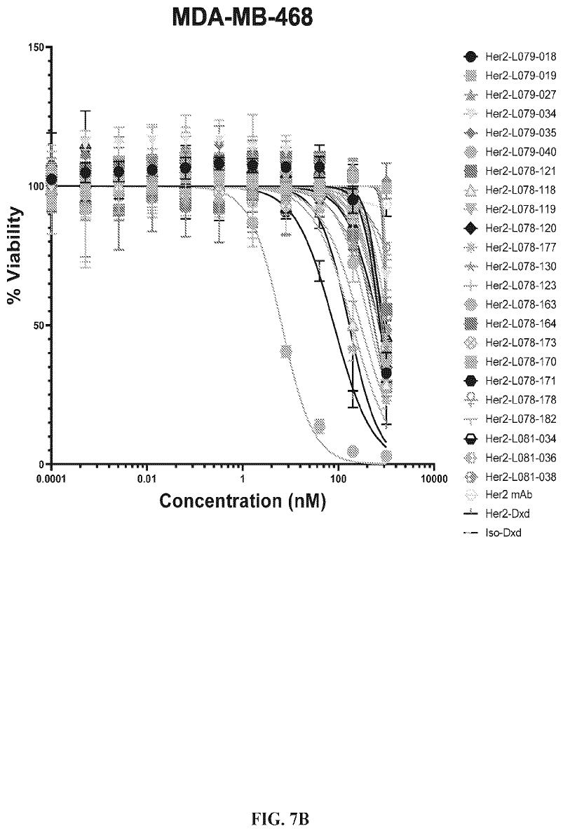

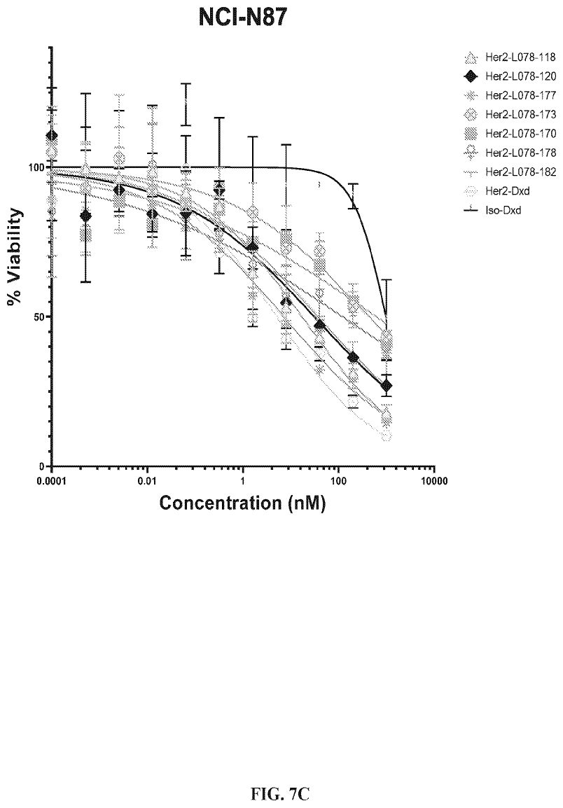

Cell viability, for ADC-L078-030-LT, ADC-L078-044, ADC-L078-045, ADC-L078-055, ADC-L078-056, ADC-L078-057, ADC-L078-058, ADC-L078-059, ADC-L078-062, ADC-L078-063, ADC-L078-064, ADC-L078-065LT, ADC-L078-066LT, ADC-L079-018, ADC-L079-019, ADC-L079-027, ADC-L079-034, ADC-L079-035, ADC-L079-040, ADC-L078-121, ADC-L078-118, ADC-L078-119, ADC-L078-120, ADC-L078-177, ADC-L078-130, ADC-L078-123, ADC-L078-163, ADC-L078-164, ADC-L078-173, ADC-L078-170, ADC-L078-171, ADC-L078-178, ADC-L078-182, ADC-L081-034, ADC-L081-036, ADC-L081-038, and controls (HER2-SET0218, HER2 antibody, STI-1499-SET0218 (also labeled Iso-Dxd), and Dxd toxin) is shown in FIGS. 3A, 3B, 4A, 4B, 7A, 7B, and 7C and Tables 5, 6, and 7. STI-1499-SET0218 is isotype control for Dxd, where Dxd is conjugated to an anti-SARS-COV-2 antibody. HER2-SET0218 is described above.

In vitro cytotoxic activities and targeting specificity of the ADCs described herein were evaluated against HER2-positive SkBr-3, HER2-positive NCI-N87, and HER2-negative MDA-MB-468 cancer cell lines using standard cell viability assays. As shown in FIGS. 3A, 3B, 4A, 4B, 7A, 7B, and 7C, treatment with ADC-L078-030-LT, ADC-L078-044, ADC-L078-045, ADC-L078-055, ADC-L078-056, ADC-L078-057, ADC-L078-058, ADC-L078-059, ADC-L078-062, ADC-L078-063, ADC-L078-064, ADC-L078-065LT, ADC-L078-066LT, ADC-L079-018, ADC-L079-019, ADC-L079-027, ADC-L079-034, ADC-L079-035, ADC-L079-040, ADC-L078-121, ADC-L078-118, ADC-L078-119, ADC-L078-120, ADC-L078-177, ADC-L078-130, ADC-L078-123, ADC-L078-163, ADC-L078-164, ADC-L078-173, ADC-L078-170, ADC-L078-171, ADC-L078-178, ADC-L078-182, ADC-L081-034, ADC-L081-036, ADC-L081-038 dose-dependently reduced SkBr-3 and NCI-N87 cell viability and did not show potent activity against MDA-MB-468 cells in 5-day assays. A range in potency as determined by EC50 of ˜0.096 to >1000 nM against HER2-positive SkBr-3 cell lines and EC50 of ˜5 to >1000 nM against HER2-positive NCI-N87 cell lines was observed (Tables 5, 6, and 7).

Comparison of ADC-L078-055, ADC-L078-056, ADC-L078-058, and ADC-L078-059, for which the same linker and conjugation chemistry (L¹-L²-L³) was used, revealed large variations in potency from EC₅₀ 0.3812 nM to >1000 nM. The difference between the ADCs is the stereochemistry and position of L³ (which is a morpholine) attachment to —C(O)NH-D.

Although some ADCs, such as ADC-L078-055 and ADC-L078-058, did not show much difference in cytotoxicity between HER2-positive SkBr-3 and HER2-negative MDA-MB-468 cells, other ADCs showed over 1500× more cytotoxicity for HER2-positive SkBr-3 cells compared to HER2-negative MDA-MB-468 cells (ADC-L078-056 and ADC-L078-062).

Isotype control STI1499-SET0218 (SARS-COV-2-linker-Dxd) was >160× less active compared to HER2 targeting ADC HER2-SET0218 (HER2-linker-Dxd) indicating that cytotoxicity was driven by HER2 targeting. In HER2-negative MDA-MB-468 cells, neither HER2 antibody nor most HER2 targeting ADCs showed cytotoxicity at concentrations up to 1 μM (FIGS. 3B, 4B, 7B and tables 5, 6, and 7), although a few ADCs showed some cytotoxicity (for example, ADC-L078-059, ADC-L078-030-LT, ADC-L079-040, Her2-L078-057, Her2-L078-059, Her2-L078-064, ADC-L078-066-LT, Her2-L078-177, and ADC-L078-063 with EC50 in the range of 20 nM-310 nM). In contrast, Dxd and other unconjugated camptothecin derivatives inhibited cell proliferation across all cell lines in a dose-dependent manner with an average EC50˜0.7 nM-163 nM, regardless of HER2 expression level.

Summary of EC50 Values (nM) of anti-HER2 ADCs and controls is presented in Table 5.

TABLE 5
EC₅₀ Values (nM) of anti-HER2 ADCs in Human Tumor Cells

EC50 (nM)

| ADC number | SkBr-3 (+) | MDA-MB-468 (−) |
|---|---|---|
| HER2-L078-055 | >1000 | >1000 |
| HER2-L078-056 | 0.3812 | >1000 |
| HER2-L078-062 | 0.1920 | 870.5 |
| HER2-L078-063 | 0.4826 | 187.3 |
| HER2-SET0218 | 0.9334 | 66.79 |
| STI1499-SET0218 | 112.1 | 115.4 |
| HER2 mAb | >1000 | >1000 |
| Dxd | 3.10 | 2.55 |

Summary of EC₅₀ Values (nM) of anti-HER2 ADCs and controls is presented in Table 6.

TABLE 6
EC₅₀ Values (nM) of anti-HER2 ADCs in Human Tumor Cells

EC50 (nM)

| ADC number | SkBr-3 (+) | MDA-MB-468 (−) |
|---|---|---|
| HER2-L078-030-LT | 0.096 | 19.47 |
| HER2-L078-044 | 4.830 | >1000 |
| HER2-L078-045 | 0.4634 | >1000 |
| HER2-L078-057 | 0.7058 | 229.6 |
| HER2-L078-058 | >1000 | >1000 |
| HER2-L078-059 | 11.67 | 309.2 |
| HER2-L078-064 | 0.5972 | 280.7 |
| HER2-L078-065LT | 1.379 | >1000 |
| HER2-L078-066LT | 9.275 | 174.3 |
| HER2-SET0218 | 0.7102 | 69.63 |
| STI1499-SET0218 | 118.4 | 105.8 |
| HER2 mAb | >1000 | >1000 |
| Dxd | 3.844 | 2.566 |

Summary of EC₅₀ Values (nM) of anti-HER2 ADCs is presented in Table 7.

TABLE 7
EC₅₀ Values (nM) of anti-HER2 ADCs in Human Tumor Cells

| | EC₅₀ (nM) | | |
|---|---|---|---|
| ADC number | SkBr-3 | NCI-N87 | MDA-MB-468 |
| HER2-L079-018 | 865.00 | N/D | 735.50 |
| HER2-L079-019 | >1000 | N/D | >1000 |
| HER2-L079-027 | >1000 | N/D | >1000 |
| HER2-L079-034 | >1000 | N/D | >1000 |
| HER2-L079-035 | >1000 | N/D | >1000 |
| HER2-L079-040 | 3.73 | N/D | 6.53 |
| HER2-L078-121 | 15.33 | N/D | 1028.00 |
| HER2-L078-118 | 0.86 | 17.24 | 293.30 |
| HER2-L078-119 | 9.35 | N/D | 779.40 |
| HER2-L078-120 | 3.76 | 31.06 | 803.70 |
| HER2-L078-177 | 0.14 | 7.16 | 195.60 |
| HER2-L078-130 | 0.50 | N/D | 731.90 |
| HER2-L078-123 | 365.10 | N/D | 834.50 |
| HER2-L078-163 | 39.79 | N/D | 457.60 |
| HER2-L078-164 | 244.70 | N/D | 550.40 |
| HER2-L078-173 | 26.56 | 428.50 | 799.70 |
| HER2-L078-170 | 30.24 | 621.60 | 764.30 |
| HER2-L078-171 | 787.10 | N/D | 706.80 |
| HER2-L078-178 | 0.54 | 122.10 | >1000 |
| HER2-L078-182 | 1.95 | 38.48 | >1000 |
| HER2-L081-034 | 3.80 | 895 | N/D |
| HER2-L081-036 | 4.95 | >1000 | N/D |
| HER2-L081-038 | 3.10 | 995 | N/D |
| HER2-Dxd | 8.52 | 5.16 | 80.71 |
| HER2 mAb | >1000 | N/D | >1000 |
| Iso-Dxd | 150.60 | 983.80 | 168.20 |

N/D was not run.

### Example B3: In Vivo Efficacy of Antibody-Drug Conjugates (ADCs)

Her2 antibody Herceptin (Trastuzumab) was purchased from Myonex LLC (Horsham, PA, USA).

Female Nu/Nu mice, 6 weeks of age, were purchased from Charles River Laboratories (Wilmington, MA). Upon receipt, groups of 5 mice were housed per cages in a controlled environment vivarium and allowed to acclimate for 72 hours prior to experimentation. Rodent chow and water were provided ad libitum. Animal health status was determined during the acclimation period. Each cage was identified by group number and study number, and mice were individually identified using ear tags. The study was conducted under approved IACUC protocols and were performed in the vivarium at Sorrento Therapeutics Inc (4955 Directors Places, San Diego, CA), which was managed by Explora BioLabs (San Diego, CA).

Animals were observed twice weekly for general clinical conditions including viability, mortality, mobility, posture, body weight (BW) and other signs of distress. If animals became moribund or lost >15% of their BW due to toxicity, or the combination of toxicity and disease, they were euthanized and recorded.

Human gastric cancer cell line NCl-N87 cells were cultured and expanded in RPMI 1640 medium (catalog #10⁻⁰⁴¹-CV, Corning, Corning, NY) supplemented with 10% FBS (catalog #FB-02, Omega Scientific, Tarzana, CA) at 37° C. in a 5% CO₂ humidified environment for a period of 2-3 weeks before harvesting for implantation. Cell viability determined by Trypan blue dye exclusion assay on a Countess II Automated Cell Counter (catalog #AMQAX1000, Invitrogen, Carlsbad, CA) and was >90% before implantation.

1.5 million of NCl-N87 cells in 100 μl of PBS (catalog #21-040-CV, Corning) -Matrigel (catalog #354234, Corning) 1:1 (v/v) mixture were implanted to the right upper flank of each mouse by s.c. injection.

Tumor volume measurement was started 11 days after tumor cell inoculation and performed twice a week after initial dosing. The longest longitudinal diameter as length and the widest transverse diameter as width were measured by using a digital caliper (catalog #62379-531, VWR, Radnor, PA). Tumor volume (TV) were then calculated by the formula: TV=[length×(width)²]/2 and were analyzed in Excel (Microsoft Office, Redmond, WA.). The mice bearing tumors between 100 mm³ and 500 mm³ were randomly assigned into each group (N=7 mice). The average tumor volume of each group was around 170 mm³.

Then mice of each group were treated with a single dose (3 mg/kg or 10 mg/kg) of PBS (vehicle), HER2 Ab (Trastuzumab) alone, or HER2-ADCs according to the regimens shown in Table 8. All compounds were diluted in PBS to working concentrations which were calculated according to treatment regimens and an injection volume of 0.2 ml per mouse.

TABLE 8
Summary of Treatment Groups and Treatment Regimens

| | | Initial Tumor | | Observation |
|---|---|---|---|---|
| | # of | Burden (mm³, | | Duration |
| Group | mice | mean ± SEM) | Treatment Regimens | (days) |
| 1 | 7 | 167.36 ± 11.36 | Vehicle/PBS, i.v., once | 28 |
| 2 | 7 | 165.54 ± 11.51 | Her2 Ab (Trastuzumab), | 28 |
| | | | 3 mg/kg, i.v., once | |
| 3 | 7 | 166.47 ± 10.66 | Her2-SET0218 (DAR4), | 28 |
| | | | 3 mg/kg, i.v., once | |
| 4 | 7 | 165.69 ± 8.77 | Her2-L078-118, | 28 |
| | | | 3 mg/kg, i.v., once | |
| 5 | 7 | 164.87 ± 11.13 | Her2-L078-120, | 28 |
| | | | 3 mg/kg, i.v., once | |
| 6 | 7 | 166.44 ± 10 | Her2-L078-170, | 28 |
| | | | 3 mg/kg, i.v., once | |
| 7 | 7 | 163.51 ± 9.31 | Her2-L078-173, | 28 |
| | | | 3 mg/kg, i.v., once | |
| 8 | 7 | 163.6 ± 9.45 | Her2-L078-177, | 28 |
| | | | 3 mg/kg, i.v., once | |
| 9 | 7 | 163.63 ± 10.96 | Her2-L078-178, | 28 |
| | | | 3 mg/kg, i.v., once | |
| 10 | 7 | 166.17 ± 10.96 | Her2-L078-182, | 28 |
| | | | 3 mg/kg, i.v., once | |
| 11 | 7 | 166.61 ± 11.34 | Her2-SET0218 (DAR4), | 28 |
| | | | 10 mg/kg, i.v., once | |
| 12 | 7 | 163.44 ± 9.54 | Her2-L078-118, | 28 |
| | | | 10 mg/kg, i.v., once | |
| 13 | 7 | 166.37 ± 10.56 | Hcr2-L078-120, | 28 |
| | | | 10 mg/kg, i.v., once | |
| 14 | 7 | 167.1 ± 11.29 | Her2-SET0218 (DAR8), | 28 |
| | | | 3 mg/kg, i.v., once | |

Tumor growth curves were plotted using GraphPad Prism 8.0 (GraphPad Software, La Jolla, CA) and values were presented as mean±SEM.

TV (tumor volume) was calculated as follows: TV=[length×(width)²]/2, where length is the longest longitudinal diameter and width is the widest trans-verse diameter.

TV change % was calculated as follows: TV change %=(TV_{dx}−TV_{d0})/TV_{d0}×100, where TV_{dx} is the TV at day x after initial treatment, and TV_{d0} is the TV just before initial treatment.

Tumor regression rate (TR %) was calculated as follows: TR %=(1−TV_{dx}/TV_{d0})×100, where TV_{dx} is the TV at day x after initial treatment, and TV_{d0} is the TV just before initial treatment.

Tumor growth inhibition rate (TGI %) was calculated as follows: TGI %=[1−(TV_{dx}−TV_{d0})ₜᵣₑₐₜₘₑₙₜ/(TV_{dx}−TV_{d0})_{control}]×100, where (TV_{dx}−TV_{d0})ₜᵣₑₐₜₘₑₙₜ is the TV change of treatment group, and (TV_{dx}−TV_{d0})_{control} is the TV change of control group. Two-way ANOVA with multiple comparison with control groups was used for statistical analyses. P<0.05 was considered as statistically significant. Levels of significance are categorized as * p<0.05, ** p<0.01, ***p<0.001, ****p<0.0001.

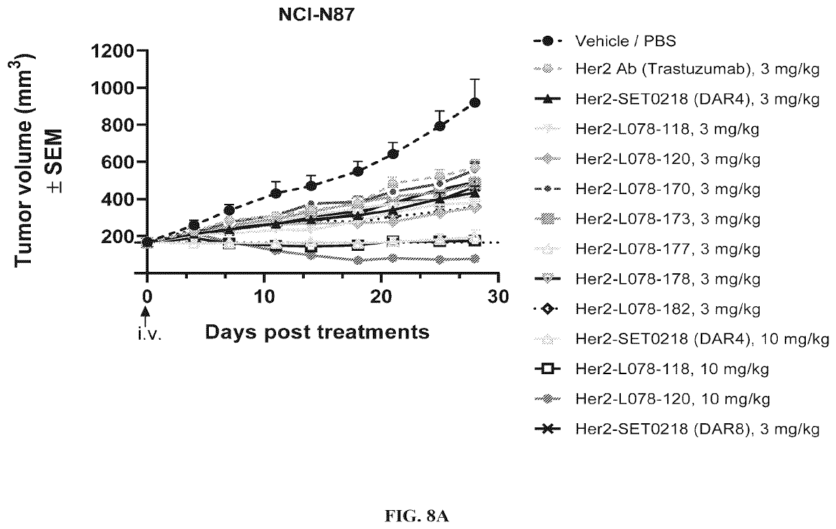

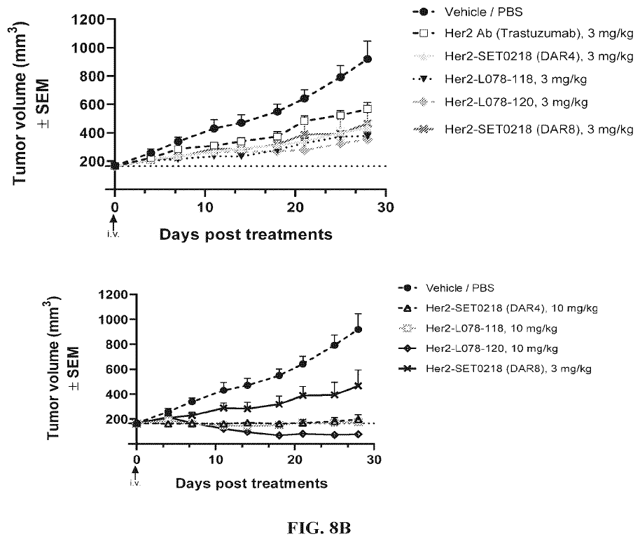

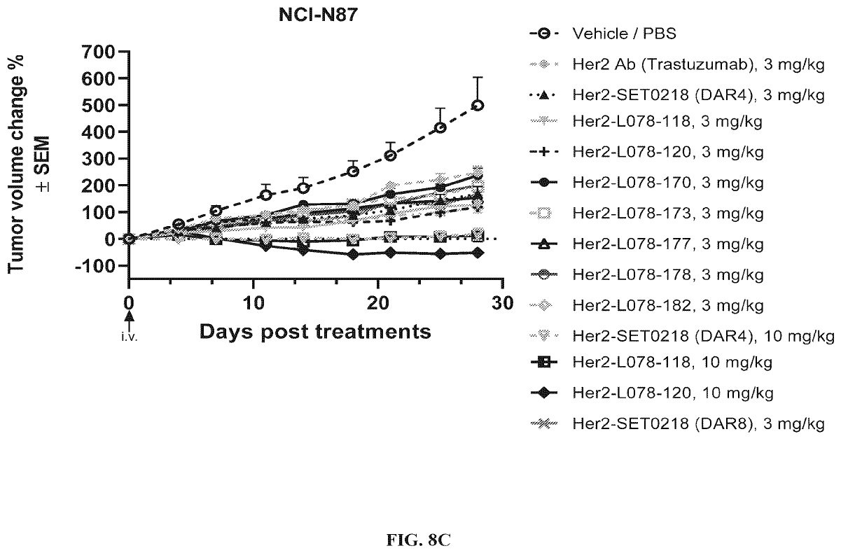

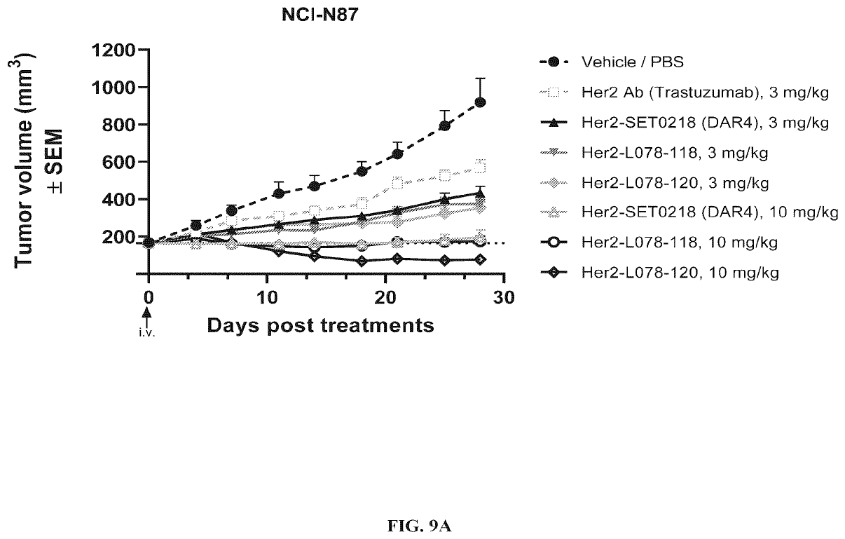

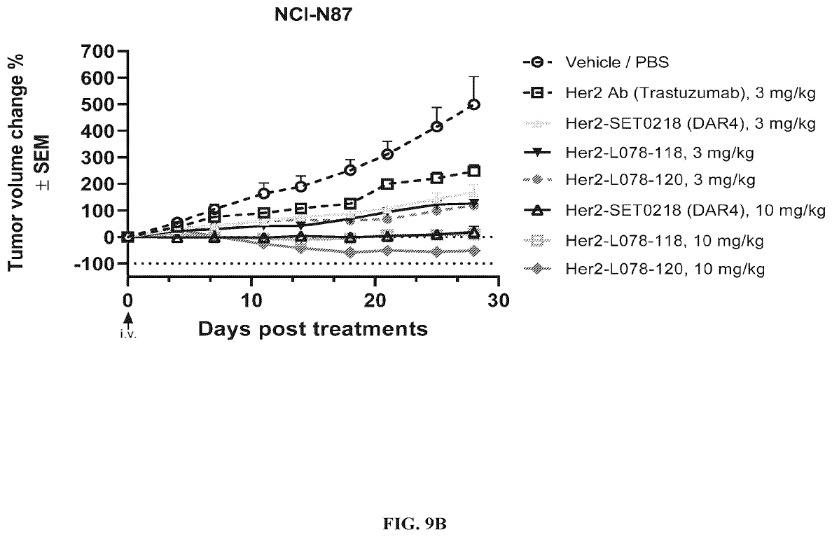

The tumor growth curves of all groups are shown in FIGS. 8A, 8B, and 9A.

Table 9 summarizes the data for tumor growth inhibition (TGI %) and tumor regression (TR %) on day 28 after treatment. Statistical analysis is shown in Table 10A and 10B.

Her2-Ab (Trastuzumab) alone and all Her2 ADCs significantly and quickly inhibited the growth of NCl-N87 tumors, after 1-3 weeks of treatment (as compared to vehicle PBS).

Following 3 mg/kg single dose administration, ADCs of Her2-L078-118, Her2-L078-182, Her2-L078-120 and Her2-SET0218 (positive control) had a maximum TGI of 76.3% (Day 21), 74.8% (Day 28), 77.7% (Day 14) and 64.4% (Day 28) respectively. Following 10 mg/kg single dose administration, the effect of Her2-L078-118 was comparable to Her2-SET0218 (DAR4) on NCl-N87 tumor model, while Her2-L078-120 demonstrated the strongest efficacy as evidenced by showing a much higher tumor regression rate of over 50% on day 28 after dosing.

Compared to Her2-SET0218 (Her2 ADC conjugated with Dxd), ADCs Her2-L078-118, Her2-L078-182 and Her2-L078-120 showed a dose dependent and better efficacy than Her2-SET0218 in terms of tumor growth inhibition as shown in FIGS. 8C and 9B.

Her2-L078-120 demonstrated the best efficacy among the tested ADCs with a 100% tumor growth inhibition and over 50% of tumor regression by day 28 after treatment as shown in Table 9. Body weight reduction was not observed in any of the treatment groups (data not shown). No overt signs of off-target toxicity were observed in any of the treatment groups.

TABLE 9
Summary data of TGI % and TR % on Day 28 after treatment

| Treatment Groups | TGI % (Day 28) | TR % (Day 28) |
|---|---|---|
| Low dose 3 mg/kg | | |
| Vehicle/PBS | 0.0 | 0.0 |
| Her2 Ab (Trastuzumab), 3 mg/kg | 46.5 | 0.0 |
| Her2-SET0218 (DAR4), 3 mg/kg | 64.4 | 0.0 |
| Her2-L078-118(DAR3.34), 3 mg/kg | 72.0 | 0.0 |
| Her2-L078-120, 3 mg/kg | 74.8 | 0.0 |
| Her2-L078-170, 3 mg/kg | 48.2 | 0.0 |
| Her2-L078-173, 3 mg/kg | 57.4 | 0.0 |
| Her2-L078-177, 3 mg/kg | 66.8 | 0.0 |
| Her2-L078-178, 3 mg/kg | 56.7 | 0.0 |
| Her2-L078-182, 3 mg/kg | 74.3 | 0.0 |
| Her2-SET0218 (DAR8), 3 mg/kg | 60.2 | 0.0 |
| High dose 10 mg/kg | | |
| Vehicle/PBS | 0.0 | 0.0 |
| Her2-SET0218(DAR4), 10 mg/kg | 96.0 | 12.4 |
| Her2-L078-118, 10 mg/kg | 98.6 | 15.7 |
| Her2-L078-120, 10 mg/kg | 100.0 | 51.9 |

TABLE 10A
Statistical analysis (3 mg/kg, i.v., once)

| | Day 4 | Day 7 | Day 11 | Day 14 | Day 18 | Day 21 | Day 25 | Day 28 |
|---|---|---|---|---|---|---|---|---|
| Her2 Ab (Trastuzumab) | ns | ns | ns | ns | * | ns | * | * |
| Her2-SET0218 (DAR4) | ns | * | ** | ** | ** | *** | *** | *** |
| Her2-L078-118 | ns | ** | *** | **** | *** | *** | **** | **** |
| Her2-L078-120 | ns | * | ** | *** | **** | **** | **** | **** |
| Her2-L078-170 | ns | ns | ns | ns | ns | * | ** | * |
| Her2-L078-173 | ns | ns | ns | ns | ns | * | *** | *** |
| Her2-L078-177 | ns | ns | * | * | * | ** | **** | **** |
| Her2-L078-178 | ns | ns | * | ** | ** | ** | ** | ** |
| Her2-L078-182 | ns | ns | ** | ** | *** | **** | **** | **** |
| Her2-SET0218 (DAR8) | ns | ** | *** | **** | *** | *** | **** | **** |

p value, vs PBS, two-way ANOVA with Dunnett's test on TV change % from day 0 to different time points.
n.s no significance,
* p < 0.05,
** p < 0.01,
*** p < 0.001,
**** p < 0.0001

TABLE 10B
Statistical analysis (10 mg/kg, i.v., once)

| | Day 4 | Day 7 | Day 11 | Day 14 | Day 18 | Day 21 | Day 25 | Day 28 |
|---|---|---|---|---|---|---|---|---|
| Her2-SET0218 (DAR4) | ** | **** | **** | **** | **** | **** | **** | **** |
| Her2-L078-118 | ns | **** | **** | **** | **** | **** | **** | **** |
| Her2-L078-120 | ns | **** | **** | **** | **** | **** | **** | **** |

p value, vs PBS, two-way ANOVA with Dunnett's test on TV change % from day 0 to different time points.
n.s no significance,
* p < 0.05,
** p < 0.01,
*** p < 0.001,
**** p < 0.0001

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

What is claimed is:

1. An antibody drug conjugate (ADC) of formula (I) or formula (II): 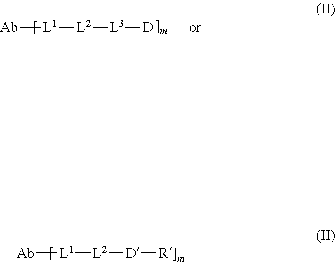
or a pharmaceutically acceptable salt thereof, wherein:
Ab is a monoclonal antibody;
m is an integer from 1 to 8;
L¹ is a linker bound to the monoclonal antibody;
L² is a bond, —C(O)—, —NH—, Amino Acid Unit, —(CH₂CH₂O)ₙ—, —(CH₂)ₙ—, —O—, -(4-aminobenzyloxycarbonyl)-, —(C(O)CH₂CH₂NH)—, —(C(O)N(R²)CH₂CH₂N(R³))—, or any combination thereof, wherein n is an integer from 1 to 24;
each R² and R³ is independently H or substituted or unsubstituted alkyl;
L³ is a substituted or unsubstituted heterocycloalkylene, substituted or unsubstituted heteroarylene, substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²;
R¹ is a substituted or unsubstituted heterocycloalkyl or a substituted or unsubstituted heteroaryl;
D is 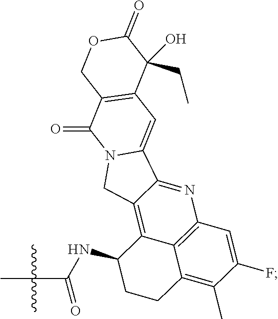 [SMILES: *C(=O)N[C@@H]1CCc2c(C)c(F)cc3nc4c(c1c23)Cn1c-4cc2c(c1=O)COC(=O)[C@@]2(O)CC]
and
D′ is 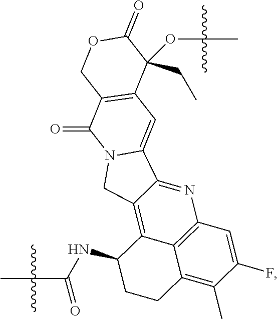
wherein D′ is connected through its amide group to R¹, and through oxygen to L².

2. The ADC or a pharmaceutically acceptable salt thereof of claim 1, wherein the monoclonal antibody is an anti-HER2 antibody.

3. The ADC or a pharmaceutically acceptable salt thereof of claim 1 or 2, wherein the monoclonal antibody is a modified monoclonal antibody.

4. The ADC or a pharmaceutically acceptable salt thereof of any one of claims 1-3, wherein L¹ is a linker bound to one or two sulfur or nitrogen atoms of the monoclonal antibody.

5. The ADC or a pharmaceutically acceptable salt thereof of any one of claims 1-4, wherein L¹ is:

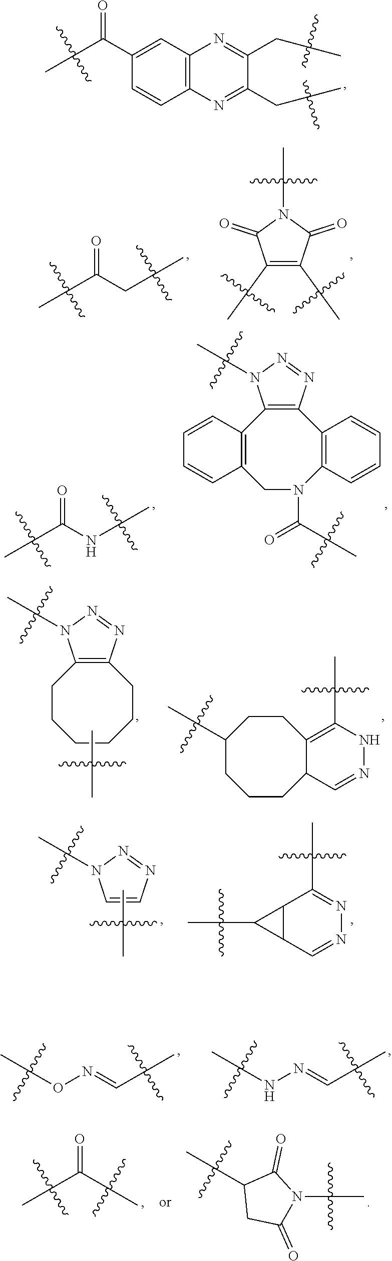

6. The ADC or a pharmaceutically acceptable salt thereof of any one of claims 1-5, wherein m is 1, 2, 3, 4, 5, 6, 7, or 8.

7. The ADC or a pharmaceutically acceptable salt thereof of any one of claims 1-6, wherein L² is a bond, —C(O)—, —NH—, -Val-, -Phe-, -Lys-, -(4-aminobenzyloxycarbonyl)-, -Gly-, -Ser-, -Thr-, -Ala-, -β-Ala-, -citrulline- (-Cit-), —(CH₂)ₙ—, —(CH₂CH₂O)ₙ—, —O—, —(C(O)N(CH₃)CH₂CH₂N(CH₃))—, or any combination thereof.

8. The ADC or a pharmaceutically acceptable salt thereof of claim 7, wherein L² is —C(O)—, —NH—, -Val-, -Ala-, -Gly-, -Cit-, -(4-aminobenzyloxycarbonyl)-, —(CH₂)ₙ—, —(CH₂CH₂O)ₙ—, —O—, —(C(O)N(CH₃)CH₂CH₂N(CH₃))—, or any combination thereof.

9. The ADC or a pharmaceutically acceptable salt thereof of claim 7, wherein L² is —C(O)—, —NH—, -Gly-, —(CH₂)ₙ—, —(CH₂CH₂O)ₙ—, or any combination thereof.

10. The ADC or a pharmaceutically acceptable salt thereof of claim 7, wherein L² is:

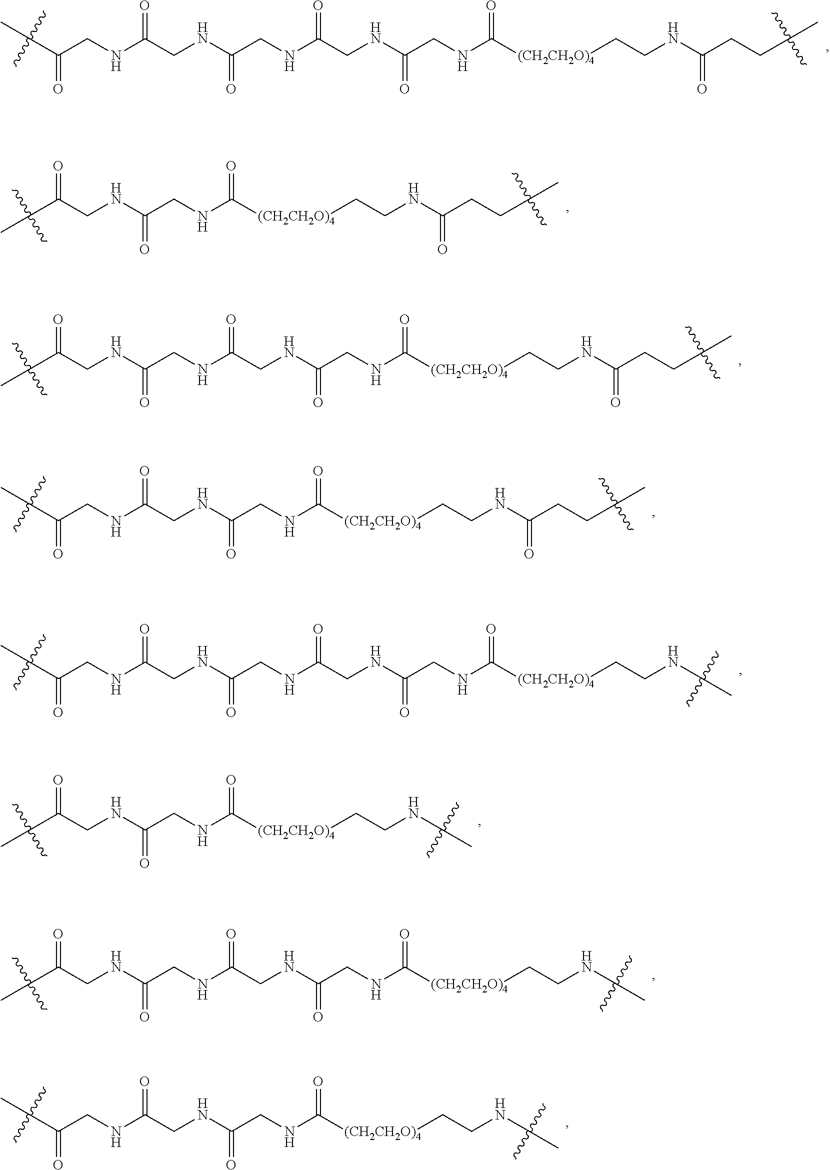

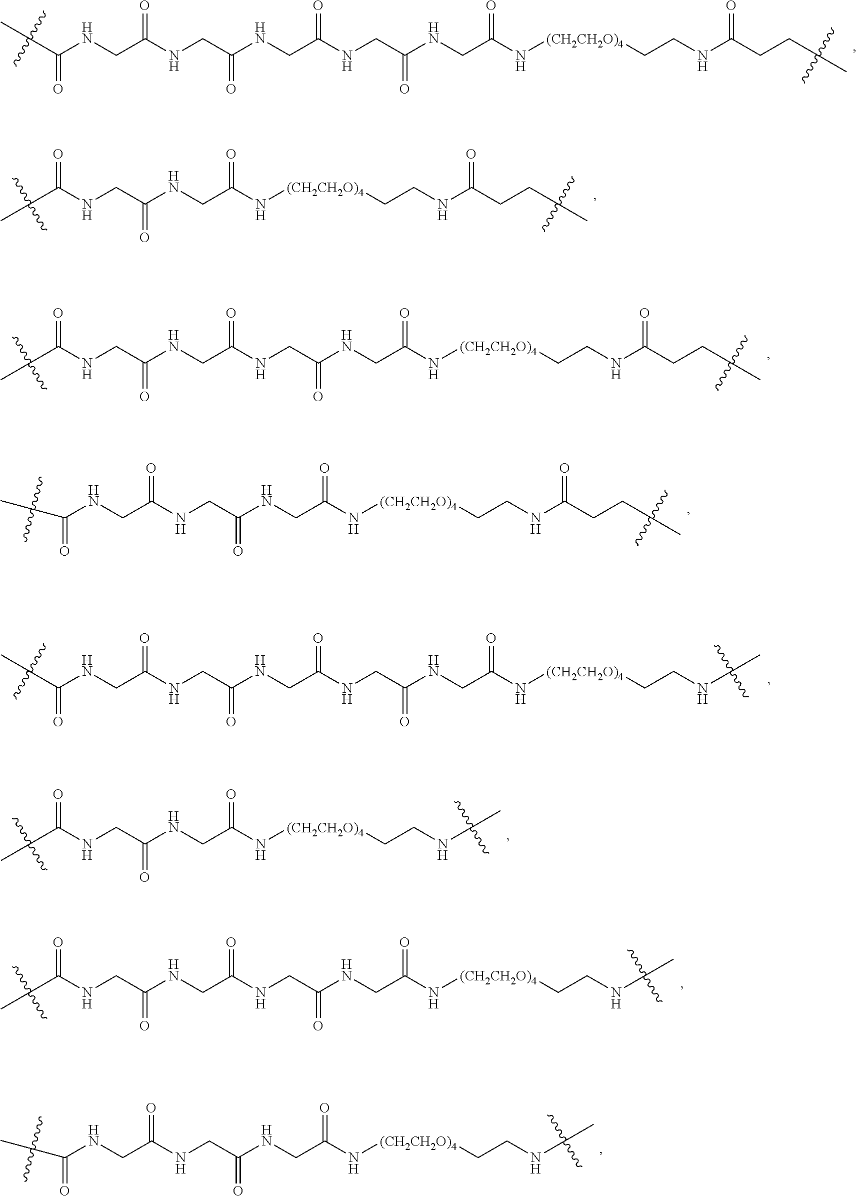

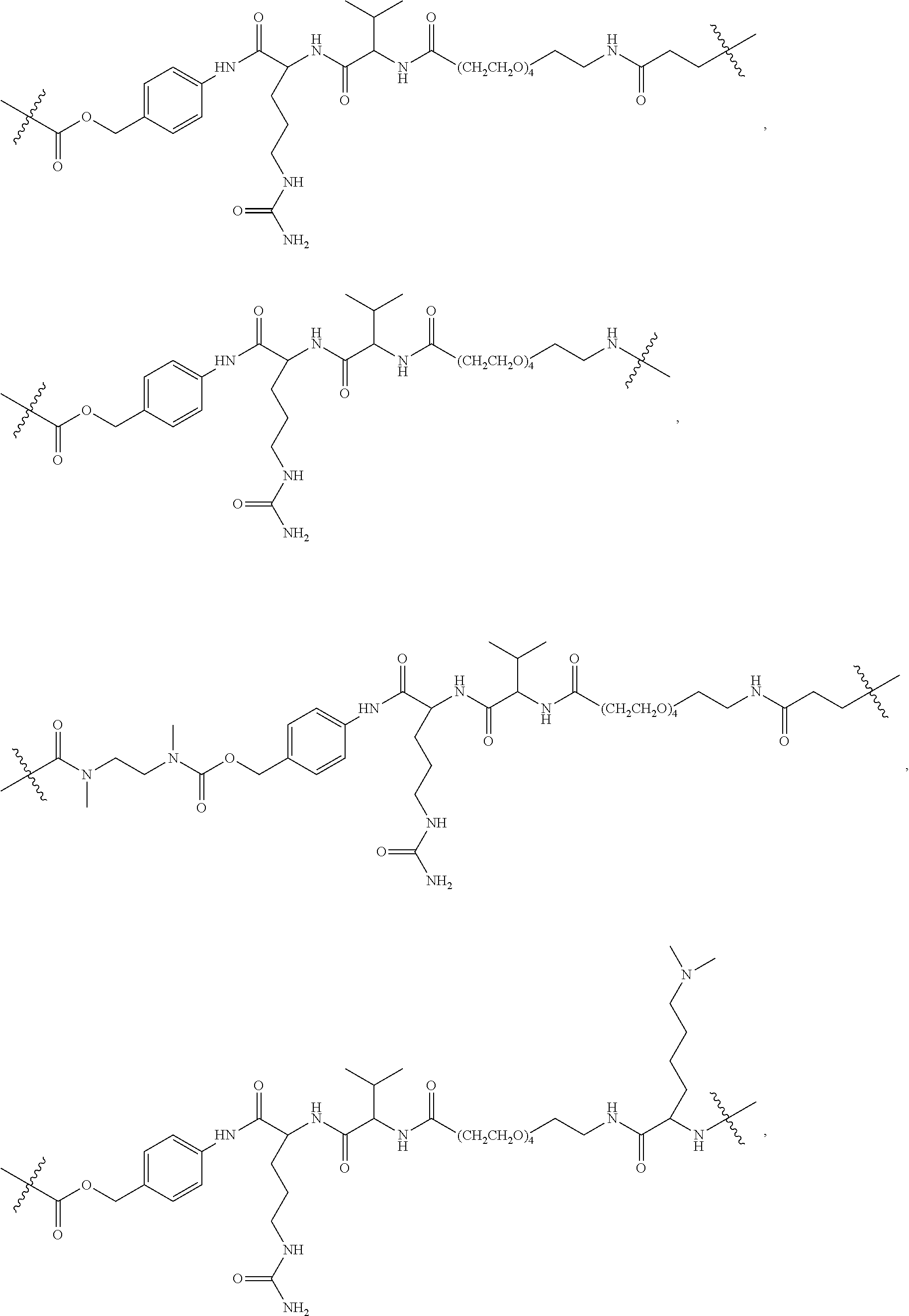

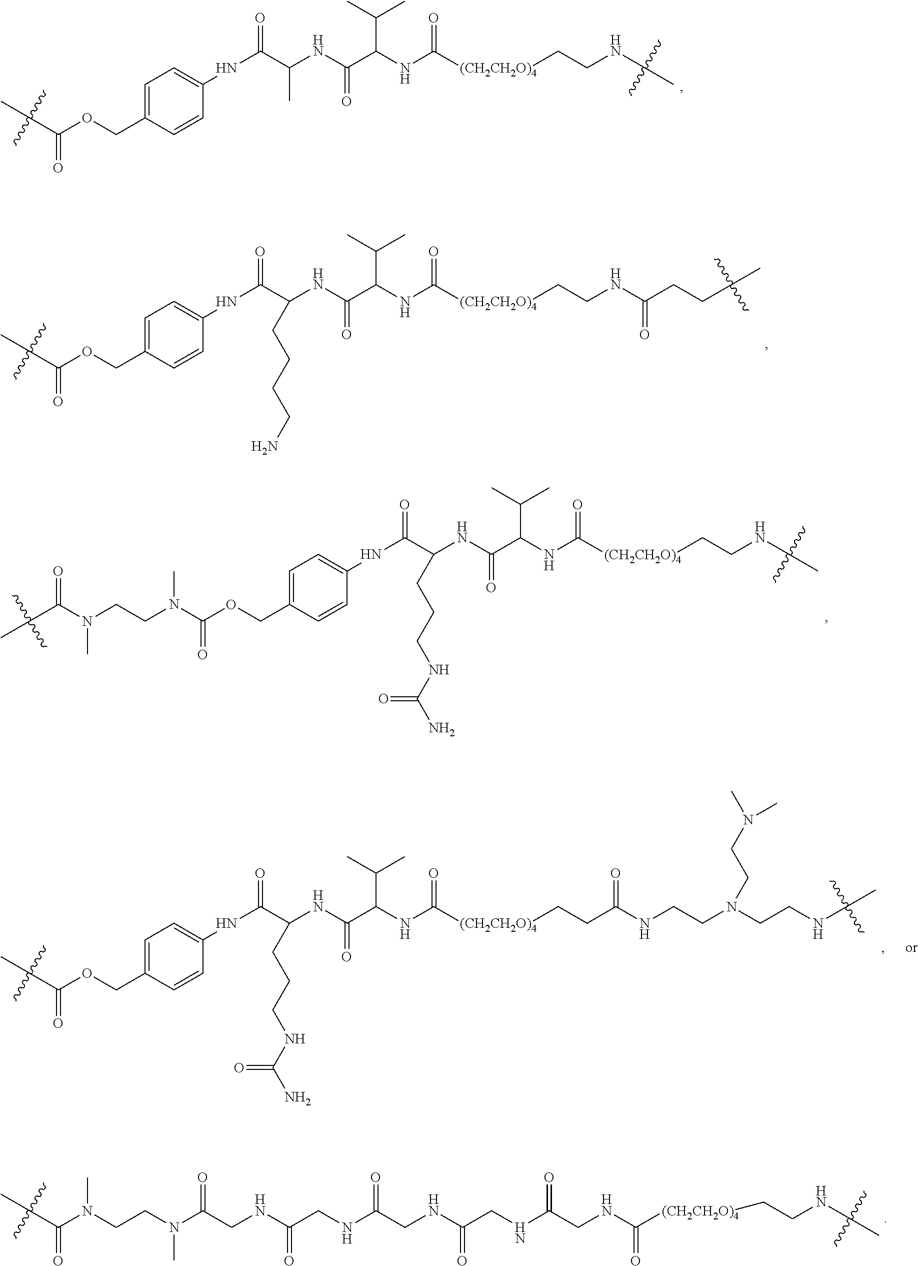

11. The ADC or a pharmaceutically acceptable salt thereof of claim 10, wherein L² is

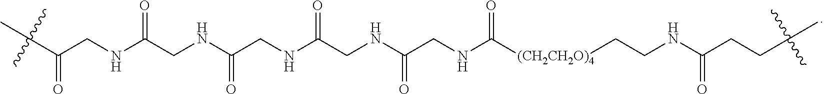

12. The ADC or a pharmaceutically acceptable salt thereof of claim 10, wherein L² is

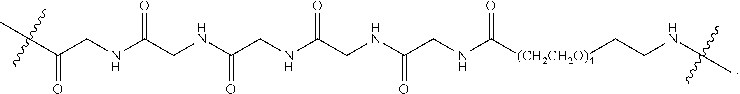

13. The ADC or a pharmaceutically acceptable salt thereof of claim 10, wherein L² is

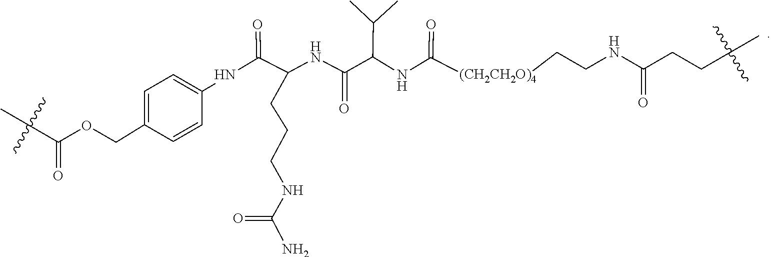

14. The ADC or a pharmaceutically acceptable salt thereof of claim 10, wherein L² is

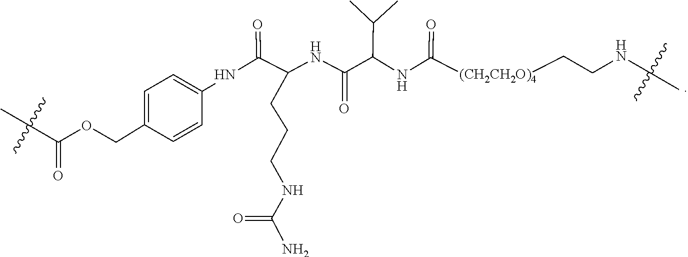

15. The ADC or a pharmaceutically acceptable salt thereof of claim 10, wherein L² is

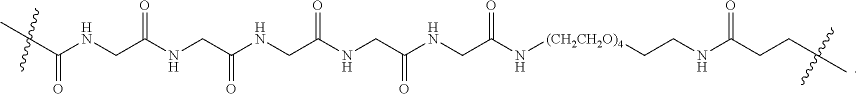

16. The ADC or a pharmaceutically acceptable salt thereof of claim 10, wherein L² is

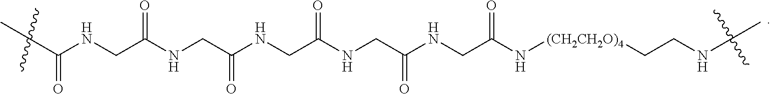

17. The ADC or a pharmaceutically acceptable salt thereof of claim 10, wherein L² is

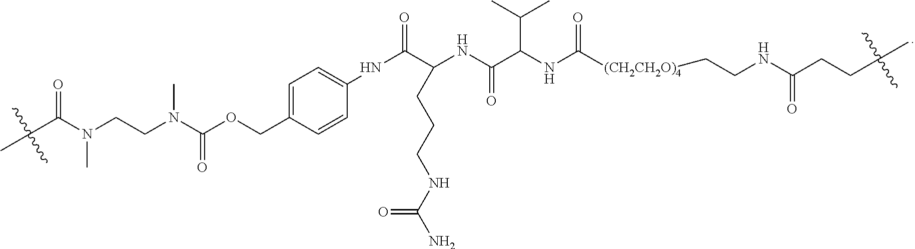

18. The ADC or a pharmaceutically acceptable salt thereof of claim 10, wherein L² is

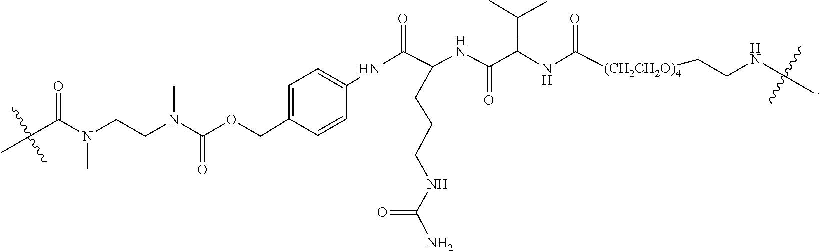

19. The ADC or a pharmaceutically acceptable salt thereof of claim 10, wherein L² is

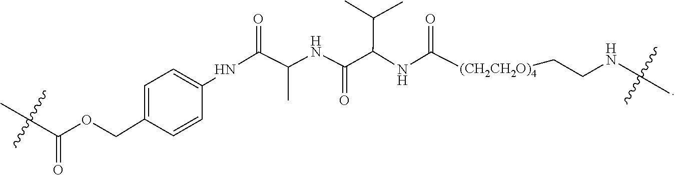

20. The ADC or a pharmaceutically acceptable salt thereof of claim 10, wherein L² is

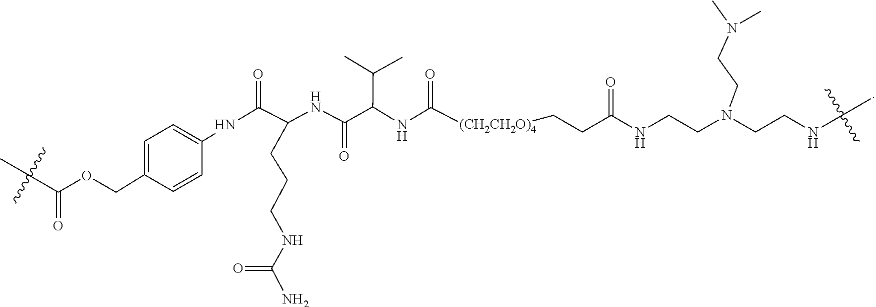

21. The ADC or a pharmaceutically acceptable salt thereof of claim 10, wherein L² is

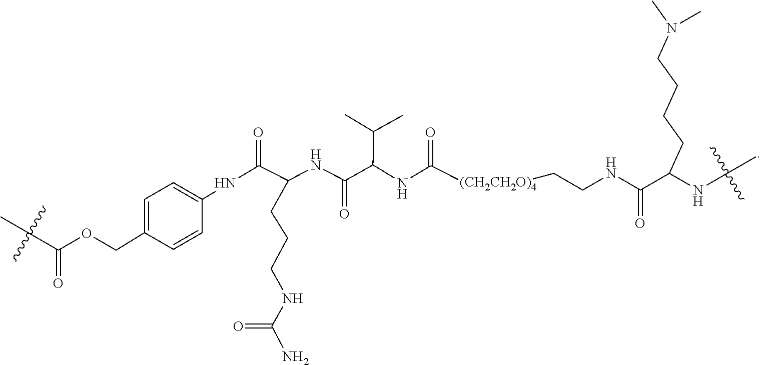

22. The ADC or a pharmaceutically acceptable salt thereof of claim 10, wherein L² is

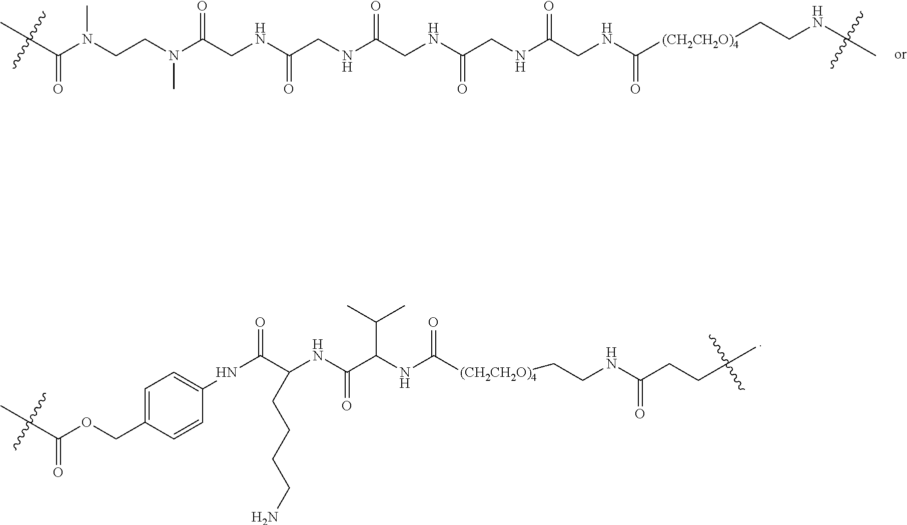

23. The ADC or a pharmaceutically acceptable salt thereof of any one of claims 1-22, wherein L³ is a substituted or unsubstituted heterocycloalkylene or substituted or unsubstituted heterocycloalkyl; or L³ is substituted or unsubstituted —OCH₂-(heterocycloalkyl), wherein L³ is linked to D through oxygen; or L³ is substituted or unsubstituted —CH₂NCH₂-(heterocycloalkyl), wherein L³ is linked to D through —CH₂—, and through nitrogen to L².

24. The ADC or a pharmaceutically acceptable salt thereof of claim 23, wherein L³ is a substituted or unsubstituted 3 to 8 membered heterocycloalkylene or substituted or unsubstituted 3 to 8 membered heterocycloalkyl; or L³ is substituted or unsubstituted —OCH₂-(3 to 8 membered heterocycloalkyl), wherein L³ is linked to D through oxygen; or L³ is substituted or unsubstituted —CH₂NCH₂-(3 to 8 membered heterocycloalkyl), wherein L³ is linked to D through —CH₂—, and through nitrogen to L².

25. The ADC or a pharmaceutically acceptable salt thereof of claim 23, wherein L³ is a substituted or unsubstituted 3 to 6 membered heterocycloalkylene or substituted or unsubstituted 3 to 6 membered heterocycloalkyl; or L³ is substituted or unsubstituted —OCH₂-(3 to 6 membered heterocycloalkyl), wherein L³ is linked to D through oxygen; or L³ is substituted or unsubstituted —CH₂NCH₂-(3 to 6 membered heterocycloalkyl), wherein L³ is linked to D through —CH₂—, and through nitrogen to L².

26. The ADC or a pharmaceutically acceptable salt thereof of claim 25, wherein L³ is a substituted or unsubstituted heterocyclobutylene, heterocyclopentylene, or heterocyclohexylene; or substituted or unsubstituted heterocyclobutyl, heterocyclopentyl, or heterocyclohexyl; or L³ is substituted or unsubstituted —OCH₂-(heterocyclobutyl, heterocyclopentyl, or heterocyclohexyl), wherein L³ is linked to D through oxygen; or L³ is substituted or unsubstituted —CH₂NCH₂-(heterocyclobutyl, heterocyclopentyl, or heterocyclohexyl), wherein L³ is linked to D through —CH₂—, and through nitrogen to L².

27. The ADC or a pharmaceutically acceptable salt thereof of any one of claims 1-22, wherein L³ is a substituted or unsubstituted heteroarylene or substituted or unsubstituted heteroaryl; or L³ is substituted or unsubstituted —OCH₂-(heteroaryl), wherein L³ is linked to D through oxygen; or substituted or unsubstituted —CH₂NCH₂-(heteroaryl), wherein L³ is linked to D through —CH₂—, and through nitrogen to L².

28. The ADC or a pharmaceutically acceptable salt thereof of claim 27, wherein L³ is a substituted or unsubstituted 5 to 10 membered heteroarylene or substituted or unsubstituted 5 to 10 membered heteroaryl; or L³ is substituted or unsubstituted —OCH₂-(5 to 10 membered heteroaryl), wherein L³ is linked to D through oxygen; or L³ is substituted or unsubstituted —CH₂NCH₂-(5 to 10 membered heteroaryl), wherein L³ is linked to D through —CH₂—, and through nitrogen to L².

29. The ADC or a pharmaceutically acceptable salt thereof of claim 27, wherein L³ is a substituted or unsubstituted 5 to 9 membered heteroarylene or substituted or unsubstituted 5 to 9 membered heteroaryl; or L³ is substituted or unsubstituted —OCH₂-(5 to 9 membered heteroaryl), wherein L³ is linked to D through oxygen; or L³ is substituted or unsubstituted —CH₂NCH₂-(5 to 9 membered heteroaryl), wherein L³ is linked to D through —CH₂—, and through nitrogen to L².

30. The ADC or a pharmaceutically acceptable salt thereof of claim 27, wherein L³ is a substituted or unsubstituted 5 to 6 membered heteroarylene or substituted or unsubstituted 5 to 6 membered heteroaryl; or L³ is substituted or unsubstituted —OCH₂-(5 to 6 membered heteroaryl), wherein L³ is linked to D through oxygen; or L³ is substituted or unsubstituted —CH₂NCH₂-(5 to 6 membered heteroaryl), wherein L³ is linked to D through —CH₂—, and through nitrogen to L².

31. The ADC or a pharmaceutically acceptable salt thereof of claim 30, wherein L³ is a substituted or unsubstituted furanylene, pyrrolylene, pyridylene, pyranylene, imidazolylene, thienylene, oxazolylene, or thiazolylene; or substituted or unsubstituted furanyl, pyrrolyl, pyridyl, pyranyl, imidazolyl, thiazolyl, thienyl, or oxazolyl; or L³ is substituted or unsubstituted —OCH₂-(furanyl, pyrrolyl, pyridyl, pyranyl, imidazolyl, thiazolyl, thienyl, or oxazolyl), wherein L³ is linked to D through oxygen; or L³ is substituted or unsubstituted —CH₂NCH₂-(furanyl, pyrrolyl, pyridyl, pyranyl, imidazolyl, thiazolyl, thienyl, or oxazolyl), wherein L³ is linked to D through —CH₂—, and through nitrogen to L².

32. The ADC or a pharmaceutically acceptable salt thereof of any one of claims 1-22, wherein R¹ is a substituted or unsubstituted heterocycloalkyl.

33. The ADC or a pharmaceutically acceptable salt thereof of claim 32, wherein R¹ is a substituted or unsubstituted 3 to 8 membered heterocycloalkyl.

34. The ADC or a pharmaceutically acceptable salt thereof of claim 32, wherein R¹ is a substituted or unsubstituted 3 to 6 membered heterocycloalkyl.

35. The ADC or a pharmaceutically acceptable salt thereof of claim 34, wherein R¹ is a substituted or unsubstituted heterocyclobutyl, heterocyclopentyl or heterocyclohexyl.

36. The ADC or a pharmaceutically acceptable salt thereof of any one of claims 1-22, wherein R¹ is a substituted or unsubstituted heteroaryl.

37. The ADC or a pharmaceutically acceptable salt thereof of claim 36, wherein R¹ is a substituted or unsubstituted 5 to 10 membered heteroaryl.

38. The ADC or a pharmaceutically acceptable salt thereof of claim 36, wherein R¹ is a substituted or unsubstituted 5 to 9 membered heteroaryl.

39. The ADC or a pharmaceutically acceptable salt thereof of claim 36, wherein R^{t} is a substituted or unsubstituted 5 to 6 membered heteroaryl.

40. The ADC or a pharmaceutically acceptable salt thereof of claim 39, wherein R^{t} is a substituted or unsubstituted furanyl, pyrrolyl, pyridyl, pyranyl, imidazolyl, or thiazolyl.

41. The ADC of any one of claims 1-22, having the structure of formula (IA) or formula (IIA): 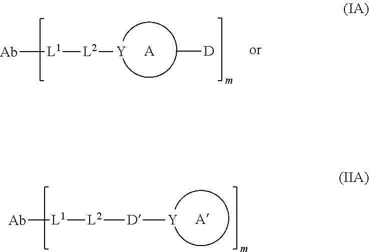
or a pharmaceutically acceptable salt thereof, wherein:
ring A is a substituted or unsubstituted heterocycloalkylene or a substituted or unsubstituted heteroarylene, connected to L² through a heteroatom Y;
ring A′ is a substituted or unsubstituted heterocycloalkyl or a substituted or unsubstituted heteroaryl, connected to D′ through a heteroatom Y; and
each Y is independently N, P, or S.

42. The ADC of claim 41, having the structure of formula (IB) or formula (IIB): 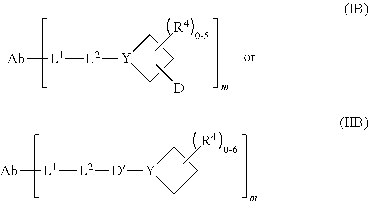
or a pharmaceutically acceptable salt thereof, wherein:
each R⁴ is independently H, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A}, —POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
any two R⁴ substituents on adjacent carbon atoms may optionally be joined to form a substituted or unsubstituted cycloalkyl, substituted or unsubstituted heterocycloalkyl, substituted or unsubstituted aryl or substituted or unsubstituted heteroaryl;
each R^{4A} and R^{4B} is independently H, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X is —Cl, —Br, —I or —F;
each n4 is independently an integer from 0 to 4;
each v4 is independently 1 or 2;
each m4 is independently an integer from 0 to 3; and
each r4 is independently 1 or 2.

43. The ADC of claim 41, having the structure of formula (IC) or formula (IIC): 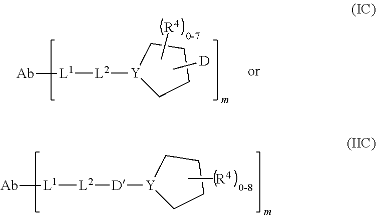
or a pharmaceutically acceptable salt thereof.

44. The ADC of claim 41, having the structure of formula (ID) or formula (IID): 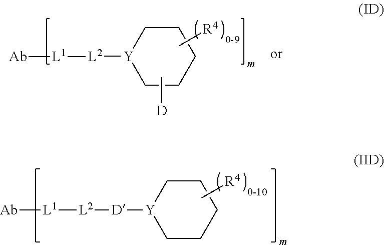
or a pharmaceutically acceptable salt thereof.

45. The ADC of claim 41, having the structure of formula (ID1) or formula (IID1): 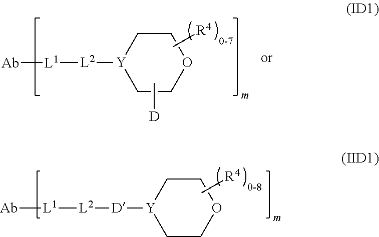
or a pharmaceutically acceptable salt thereof.

46. The ADC of claim 41, having the structure of formula (IE) or formula (IIE): 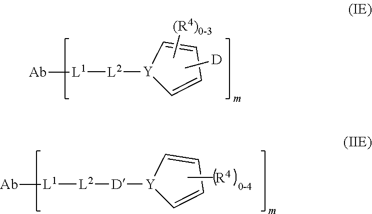
or a pharmaceutically acceptable salt thereof.

47. The ADC of claim 41, having the structure of formula (IF) or formula (IIF): 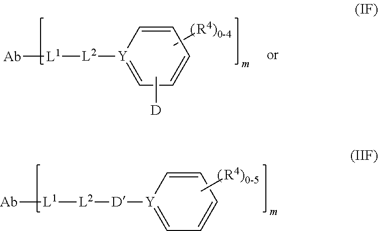
or a pharmaceutically acceptable salt thereof.

48. The ADC of any one of claims 1-22, having the structure of formula IG or IH: 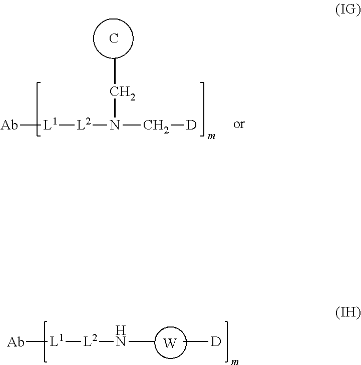
or a pharmaceutically acceptable salt thereof, wherein:
ring W is a substituted or unsubstituted cycloalkylene or a substituted or unsubstituted arylene;
ring C is a substituted or unsubstituted cycloalkyl, substituted or unsubstituted heterocycloalkyl, substituted or unsubstituted aryl, or substituted or unsubstituted heteroaryl.

49. The ADC of claim 44, having the structure of formula IJ or IK: 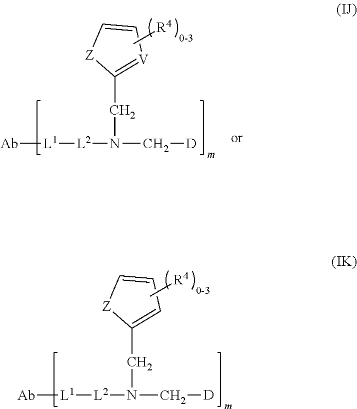
or a pharmaceutically acceptable salt thereof, wherein:
Z is S, N, or O; and
V is C or N.

50. The ADC of claim 44, having the structure of formula IL or IM: 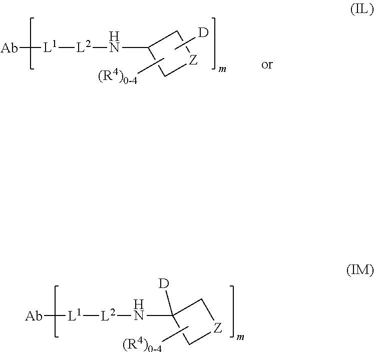
or a pharmaceutically acceptable salt thereof.

51. The ADC of claim 44, having the structure of formula IN or IO: 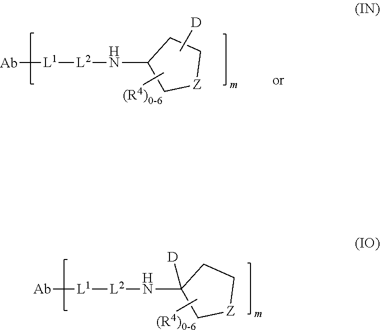
or a pharmaceutically acceptable salt thereof.

52. The ADC of claim 44, having the structure of formula IP or IQ: 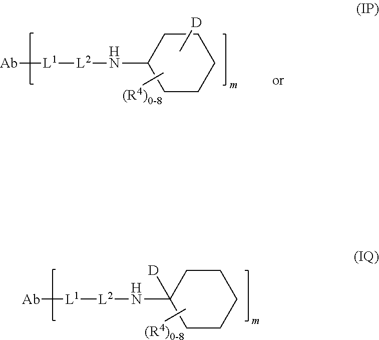
or a pharmaceutically acceptable salt thereof.

53. The ADC or a pharmaceutically acceptable salt thereof of any one of claims 1-52, wherein the ADC is: 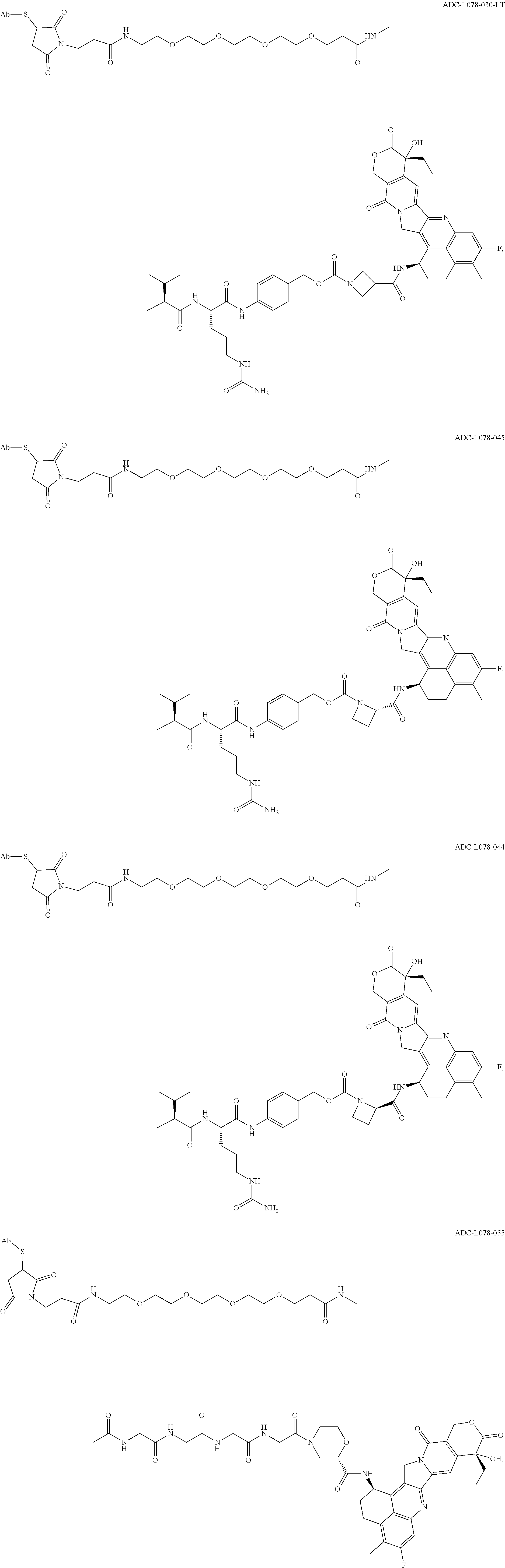 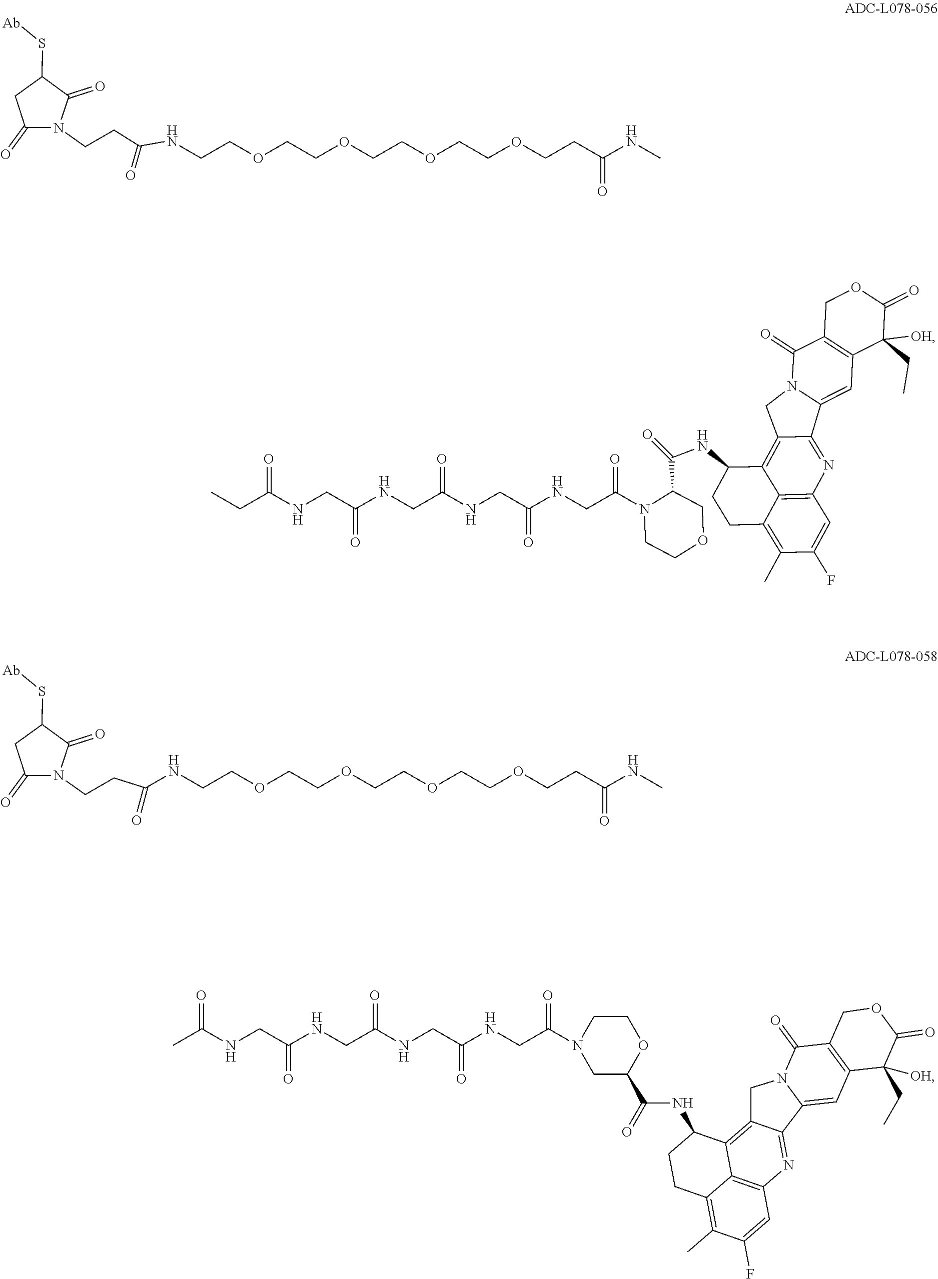 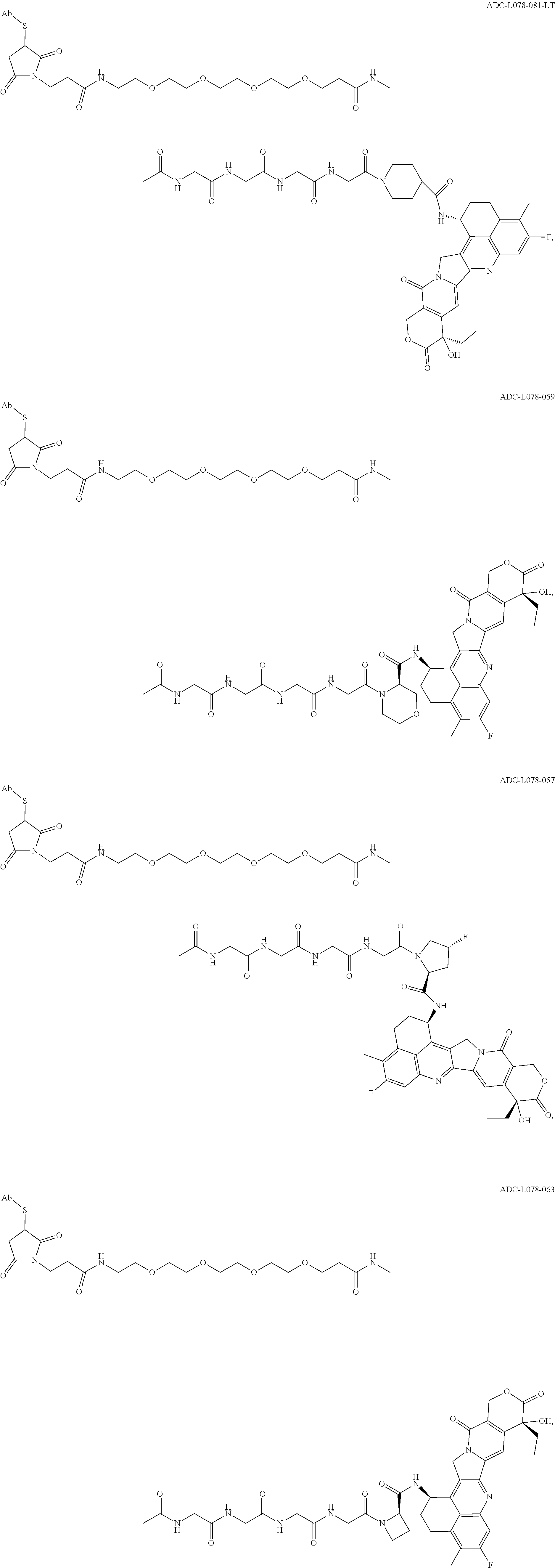 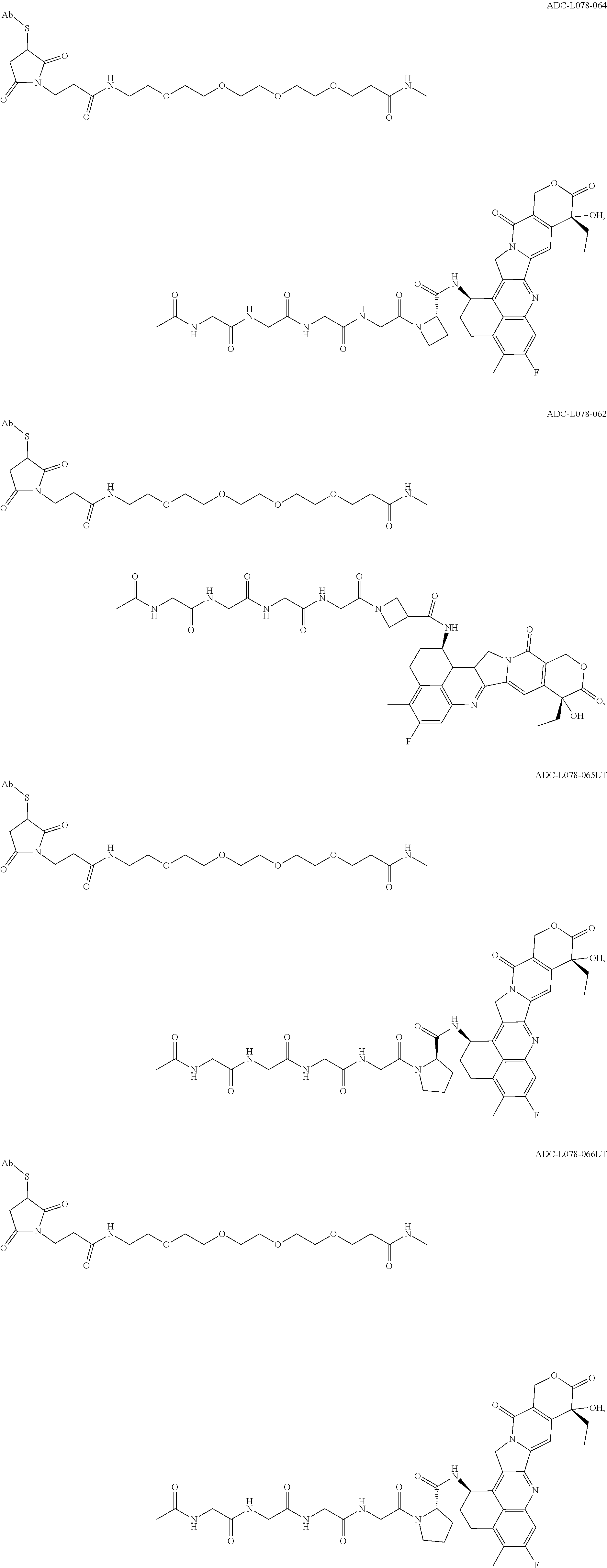 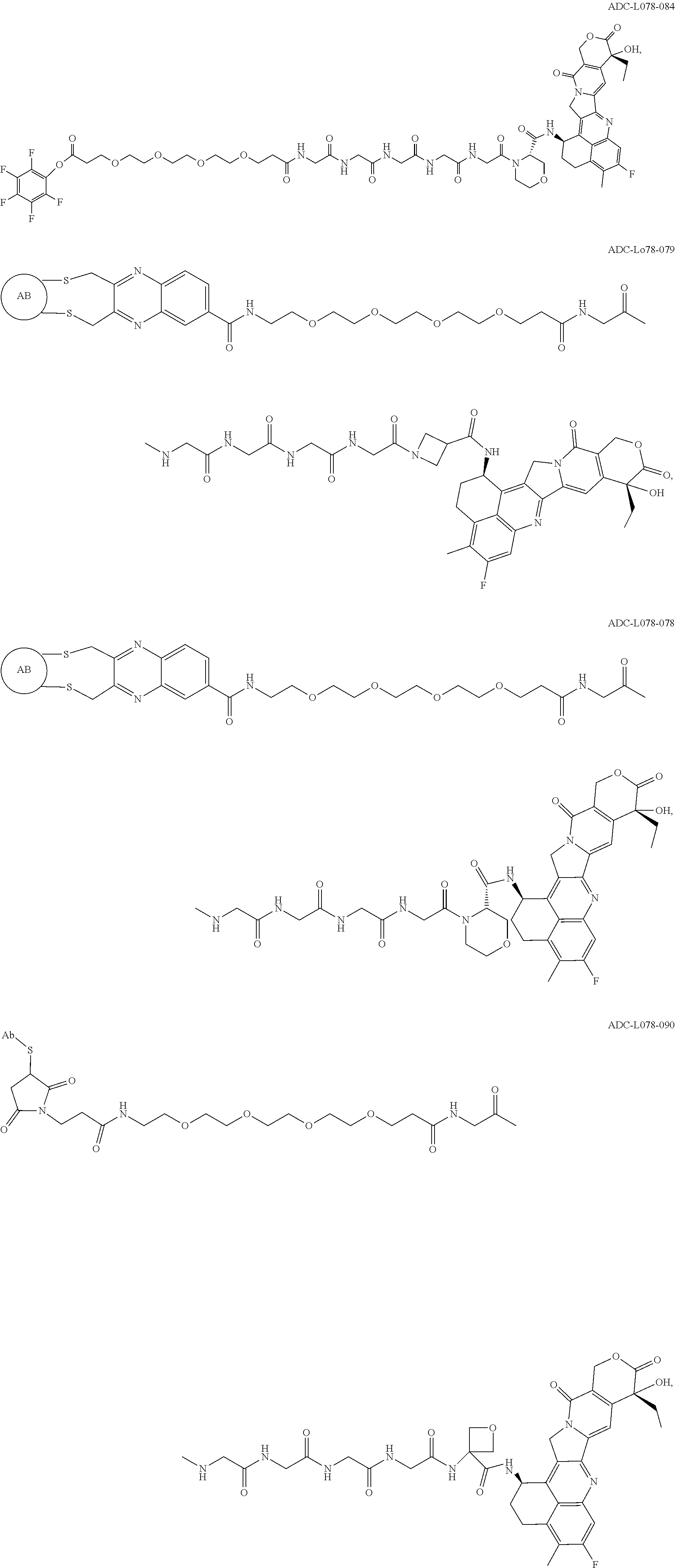 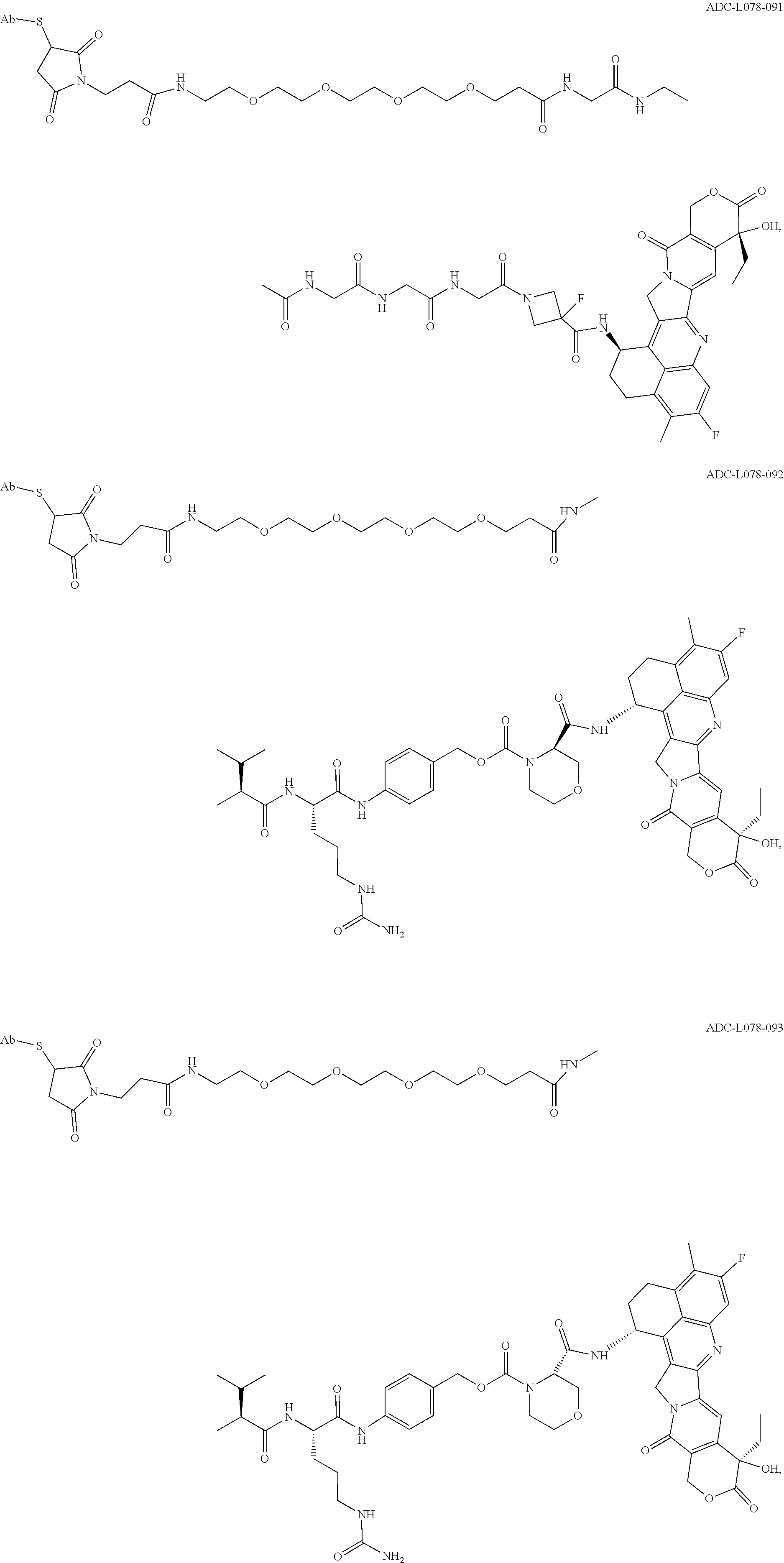 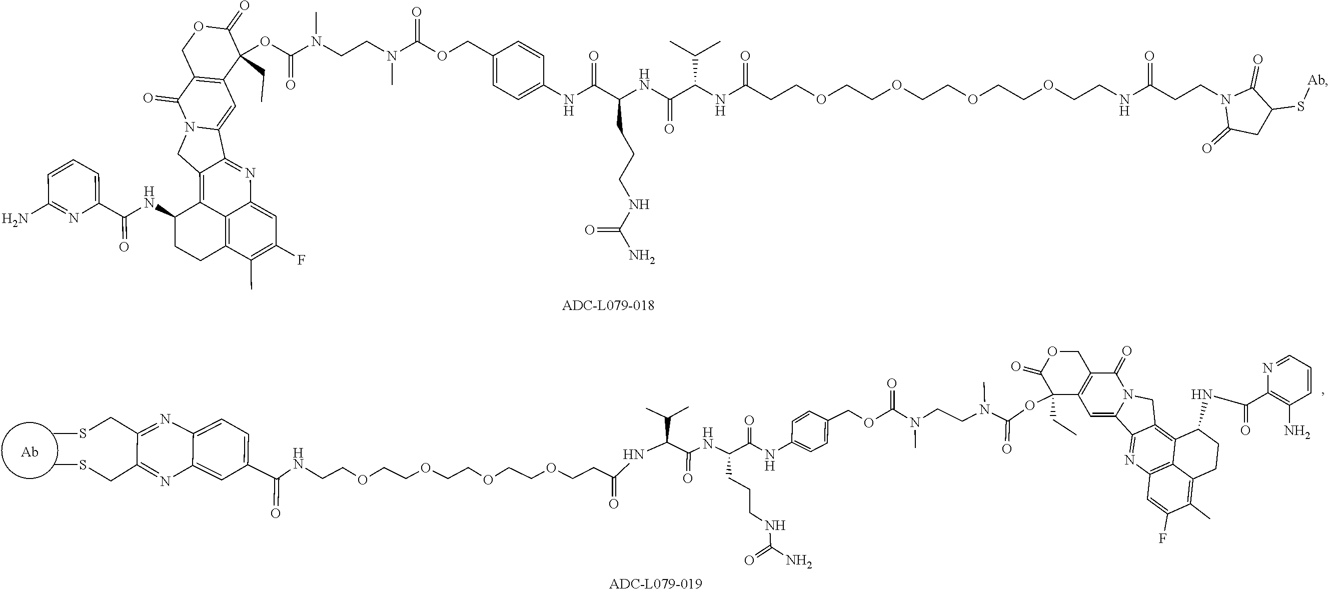 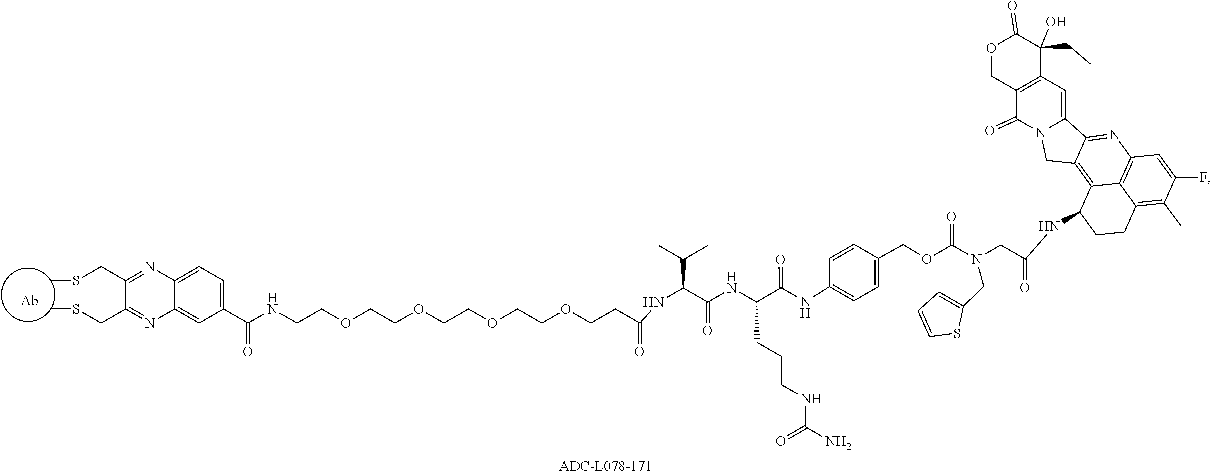 [SMILES: CC[C@]1(O)C(=O)OCc2c1cc1n(c2=O)Cc2c-1nc1cc(F)c(C)c3c1c2[C@H](NC(=O)CN(Cc1cccs1)C(=O)OCc1ccc(NC(=O)[C@H](CCCNC(N)=O)NC(=O)[C@@H](NC(=O)CCOCCOCCOCCOCCNC(=O)c2ccc4nc(CSC)c(CSC)nc4c2)C(C)C)cc1)CC3] 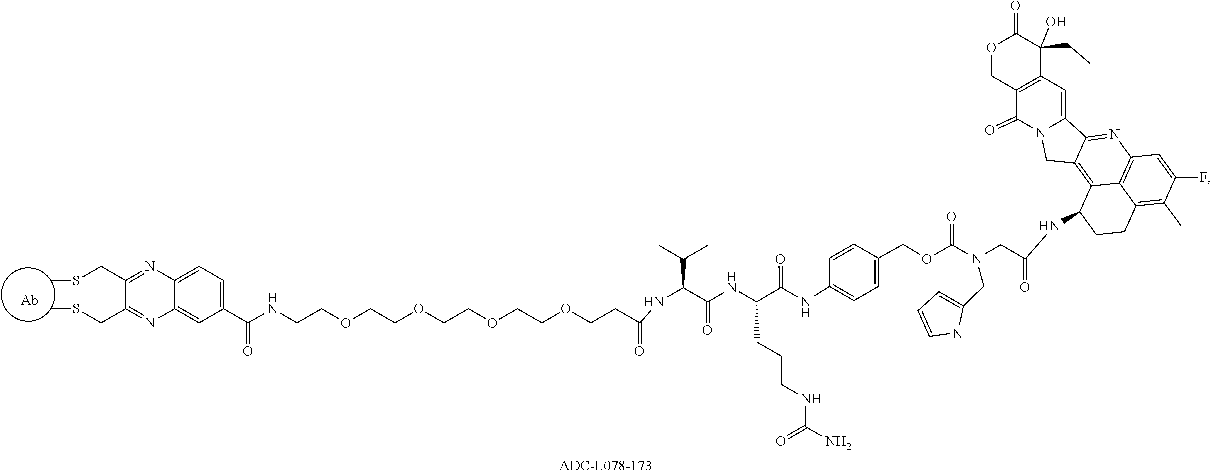 [SMILES: CC[C@]1(O)C(=O)OCc2c1cc1n(c2=O)Cc2c-1nc1cc(F)c(C)c3c1c2[C@H](NC(=O)CN(Cc1ccc[nH]1)C(=O)OCc1ccc(NC(=O)[C@H](CCCNC(N)=O)NC(=O)[C@@H](NC(=O)CCOCCOCCOCCOCCNC(=O)c2ccc4nc(CSC)c(CSC)nc4c2)C(C)C)cc1)CC3] 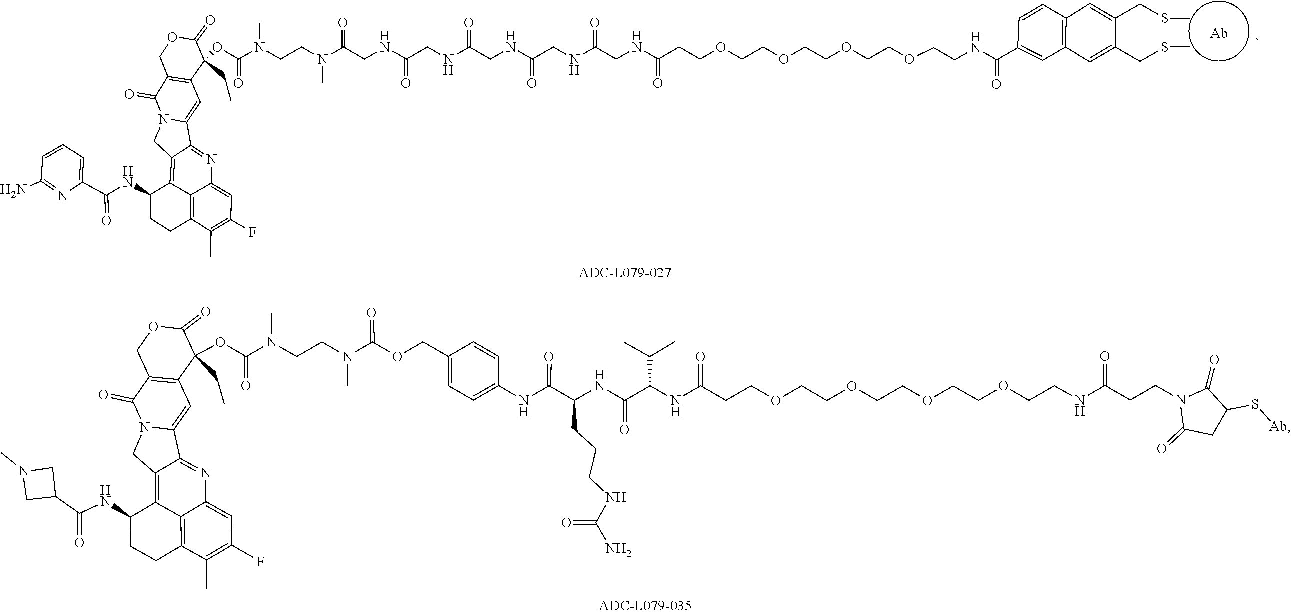 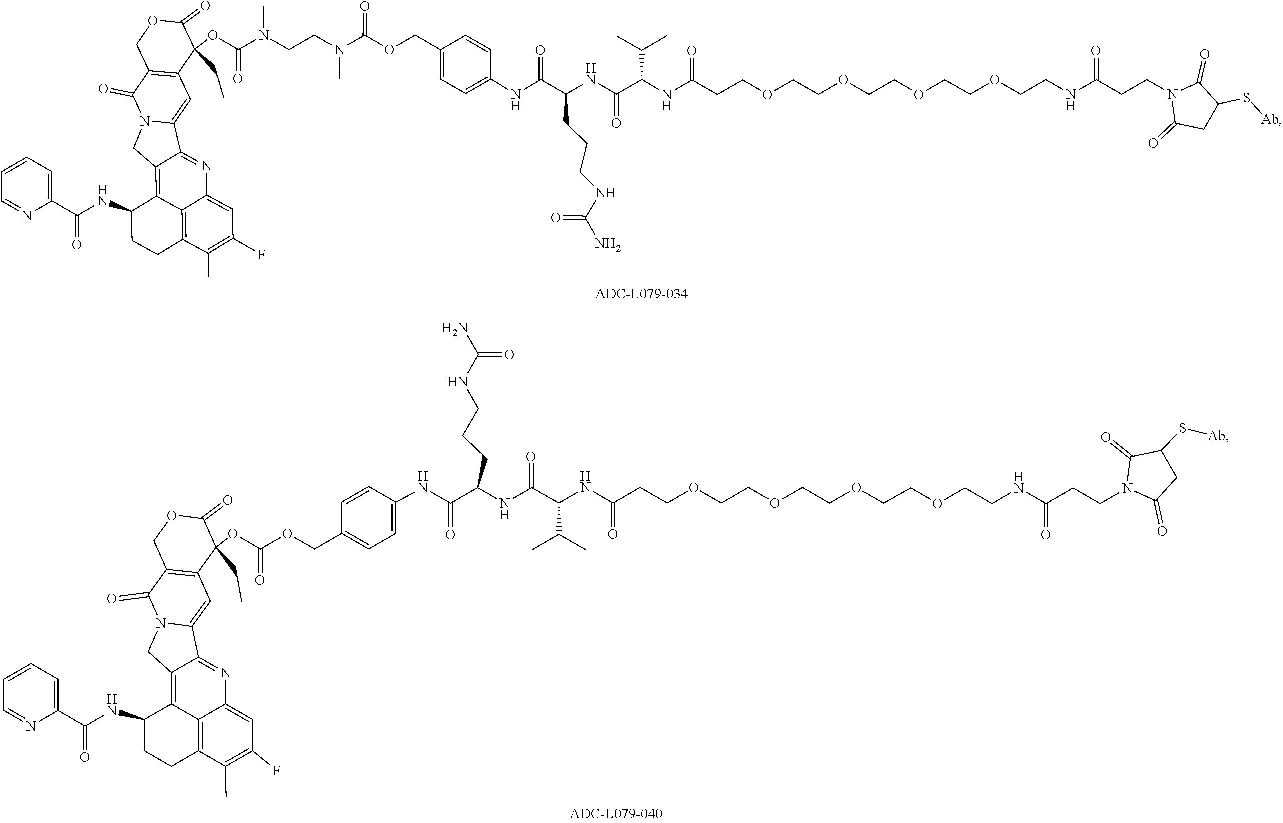 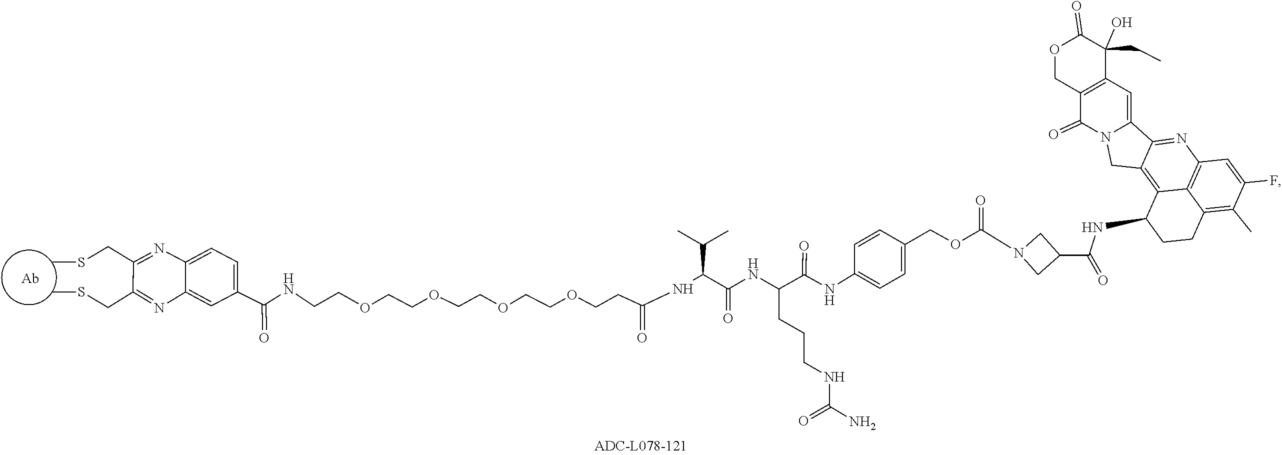 [SMILES: CC[C@]1(O)C(=O)OCc2c1cc1n(c2=O)Cc2c-1nc1cc(F)c(C)c3c1c2[C@H](NC(=O)C1CN(C(=O)OCc2ccc(NC(=O)C(CCCCNC(N)=O)NC(=O)[C@@H](NC(=O)CCOCCOCCOCCOCCNC(=O)c4ccc5nc(CSC)c(CSC)nc5c4)C(C)C)cc2)C1)CC3] 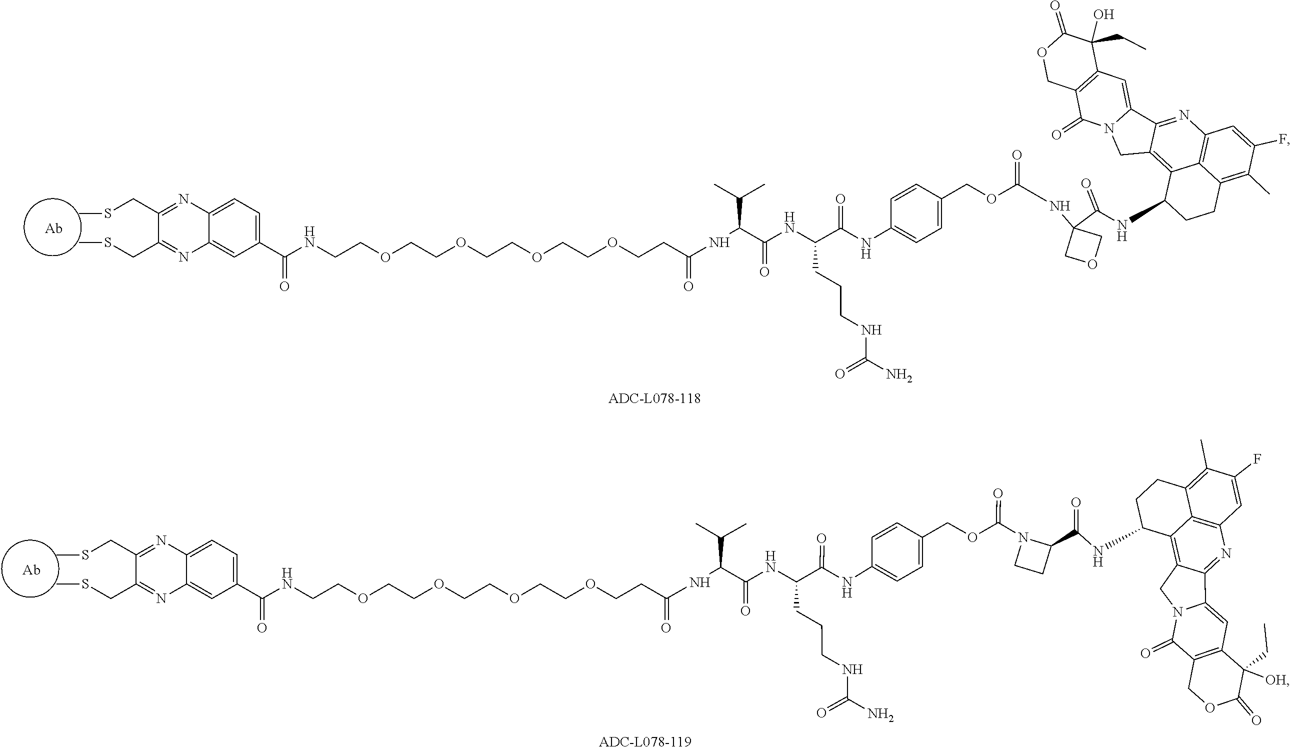 [SMILES: CC[C@]1(O)C(=O)OCc2c1cc1n(c2=O)Cc2c-1nc1cc(F)c(C)c3c1c2[C@H](NC(=O)C1(NC(=O)OCc2ccc(NC(=O)[C@H](CCCNC(N)=O)NC(=O)[C@@H](NC(=O)CCOCCOCCOCCOCCNC(=O)c4ccc5nc(CSC)c(CSC)nc5c4)C(C)C)cc2)COC1)CC3 ; CC[C@]1(O)C(=O)OCc2c1cc1n(c2=O)Cc2c-1nc1cc(F)c(C)c3c1c2[C@H](NC(=O)[C@H]1CCN1C(=O)OCc1ccc(NC(=O)[C@H](CCCNC(N)=O)NC(=O)[C@@H](NC(=O)CCOCCOCCOCCOCCNC(=O)c2ccc4nc(CSC)c(CSC)nc4c2)C(C)C)cc1)CC3] 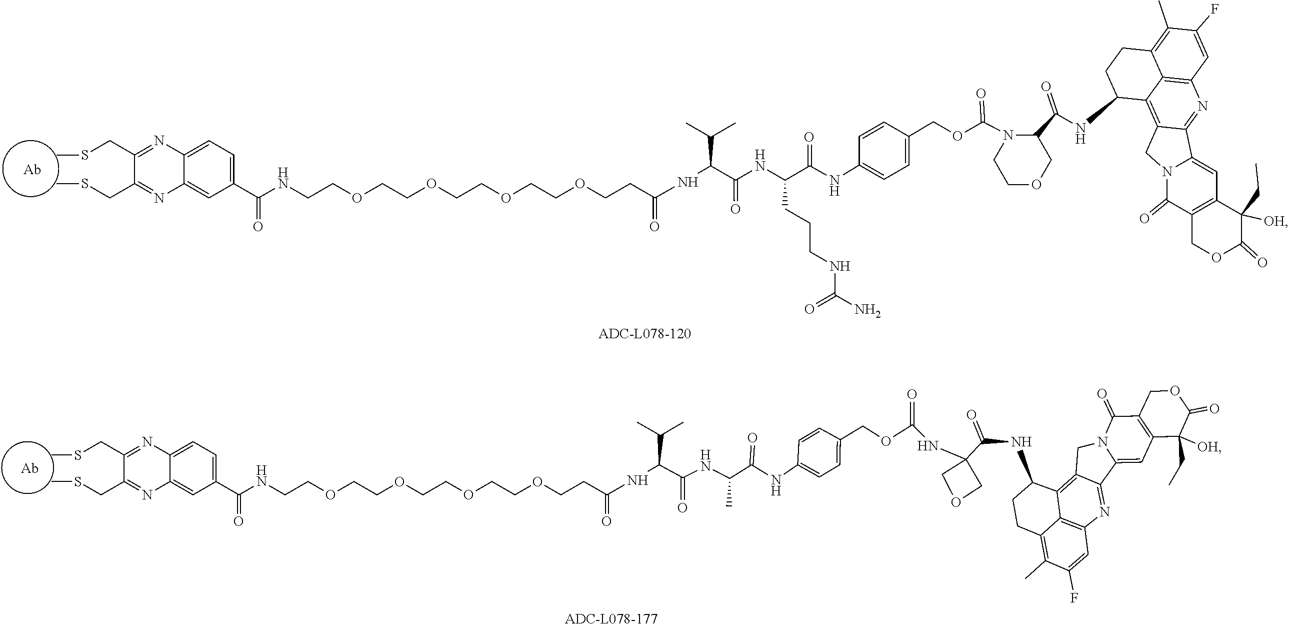 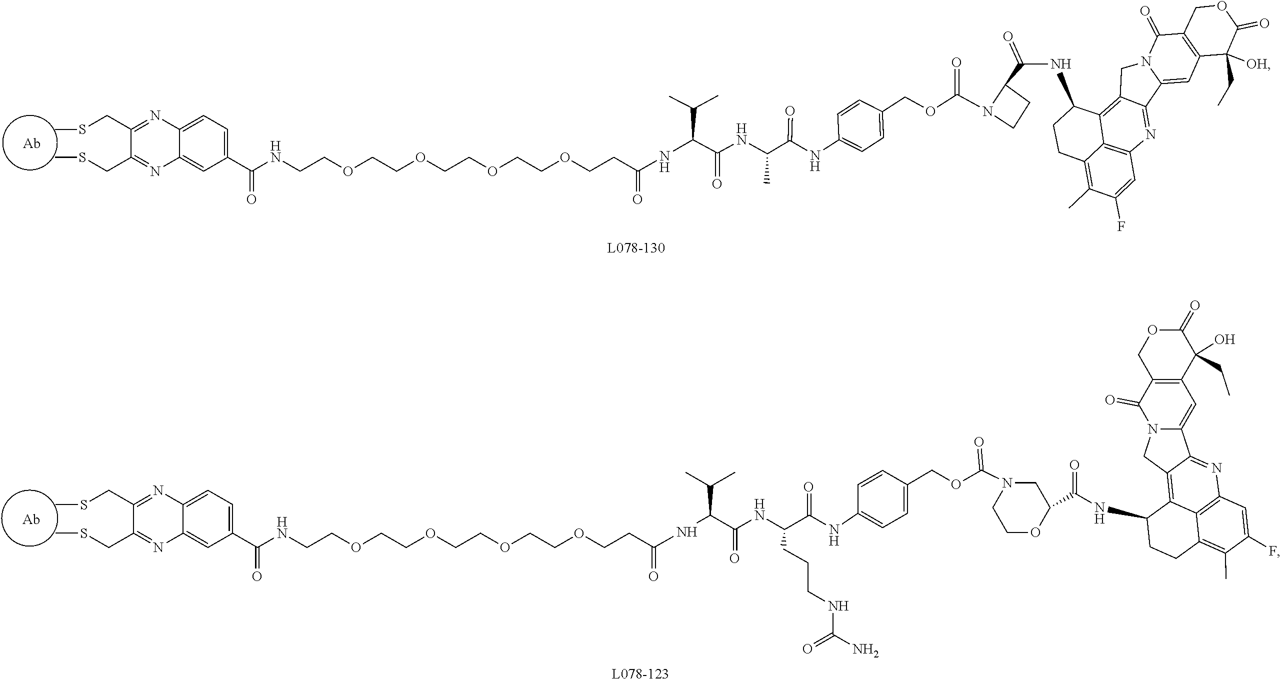 [SMILES: CC[C@]1(O)C(=O)OCc2c1cc1n(c2=O)Cc2c-1nc1cc(F)c(C)c3c1c2[C@H](NC(=O)[C@H]1CCN1C(=O)OCc1ccc(NC(=O)[C@H](C)NC(=O)[C@@H](NC(=O)CCOCCOCCOCCOCCNC(=O)c2ccc4nc(CSC)c(CSC)nc4c2)C(C)C)cc1)CC3 ; CC[C@]1(O)C(=O)OCc2c1cc1n(c2=O)Cc2c-1nc1cc(F)c(C)c3c1c2[C@H](NC(=O)[C@H]1CN(C(=O)OCc2ccc(NC(=O)[C@H](CCCNC(N)=O)NC(=O)[C@@H](NC(=O)CCOCCOCCOCCOCCNC(=O)c4ccc5nc(CSC)c(CSC)nc5c4)C(C)C)cc2)CCO1)CC3] 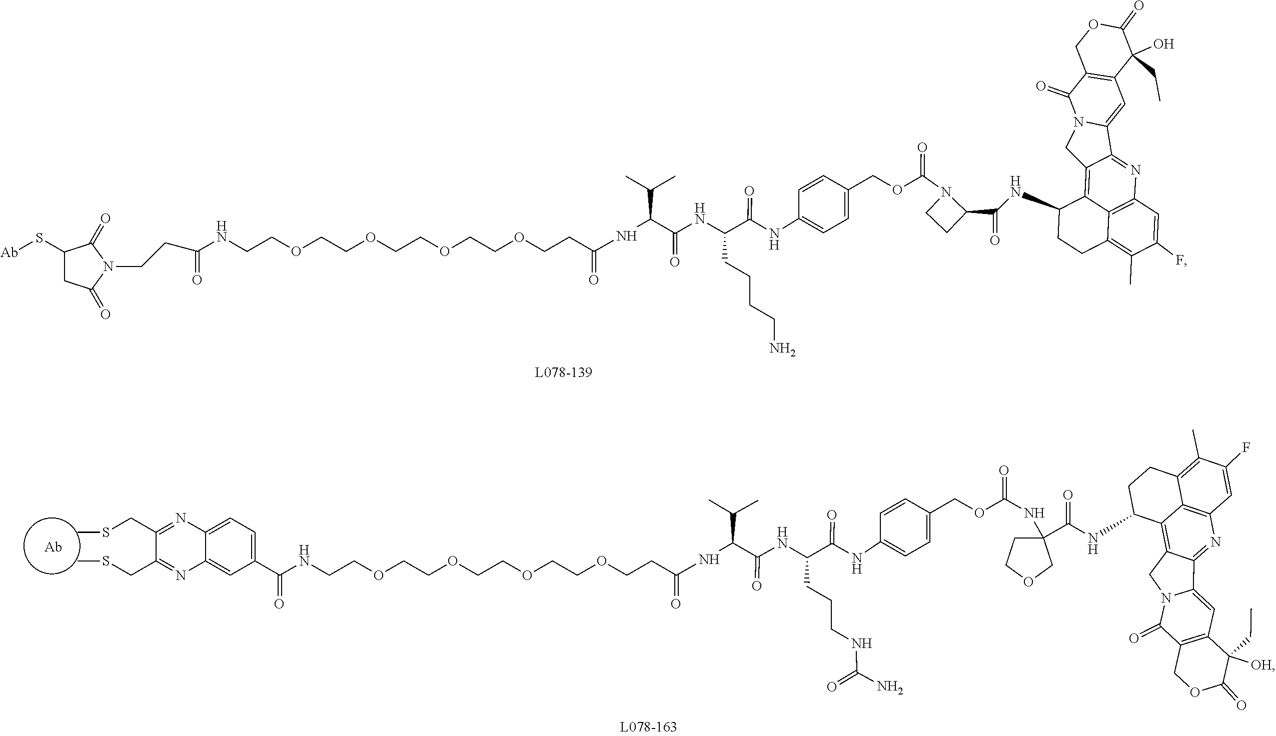 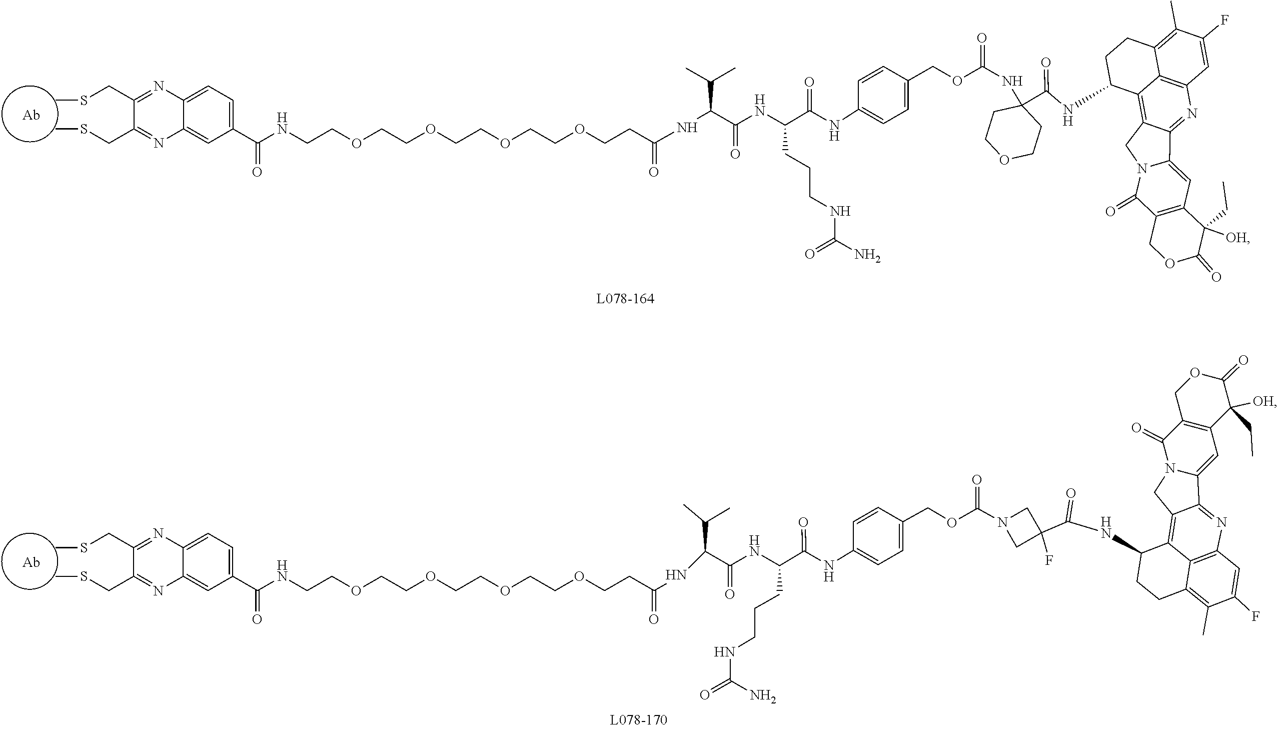 [SMILES: CC[C@]1(O)C(=O)OCc2c1cc1n(c2=O)Cc2c-1nc1cc(F)c(C)c3c1c2[C@H](NC(=O)C1(NC(=O)OCc2ccc(NC(=O)[C@H](CCCNC(N)=O)NC(=O)[C@@H](NC(=O)CCOCCOCCOCCOCCNC(=O)c4ccc5nc(CSC)c(CSC)nc5c4)C(C)C)cc2)CCOCC1)CC3 ; CC[C@]1(O)C(=O)OCc2c1cc1n(c2=O)Cc2c-1nc1cc(F)c(C)c3c1c2[C@H](NC(=O)C1(F)CN(C(=O)OCc2ccc(NC(=O)[C@H](CCCNC(N)=O)NC(=O)[C@@H](NC(=O)CCOCCOCCOCCOCCNC(=O)c4ccc5nc(CSC)c(CSC)nc5c4)C(C)C)cc2)C1)CC3] 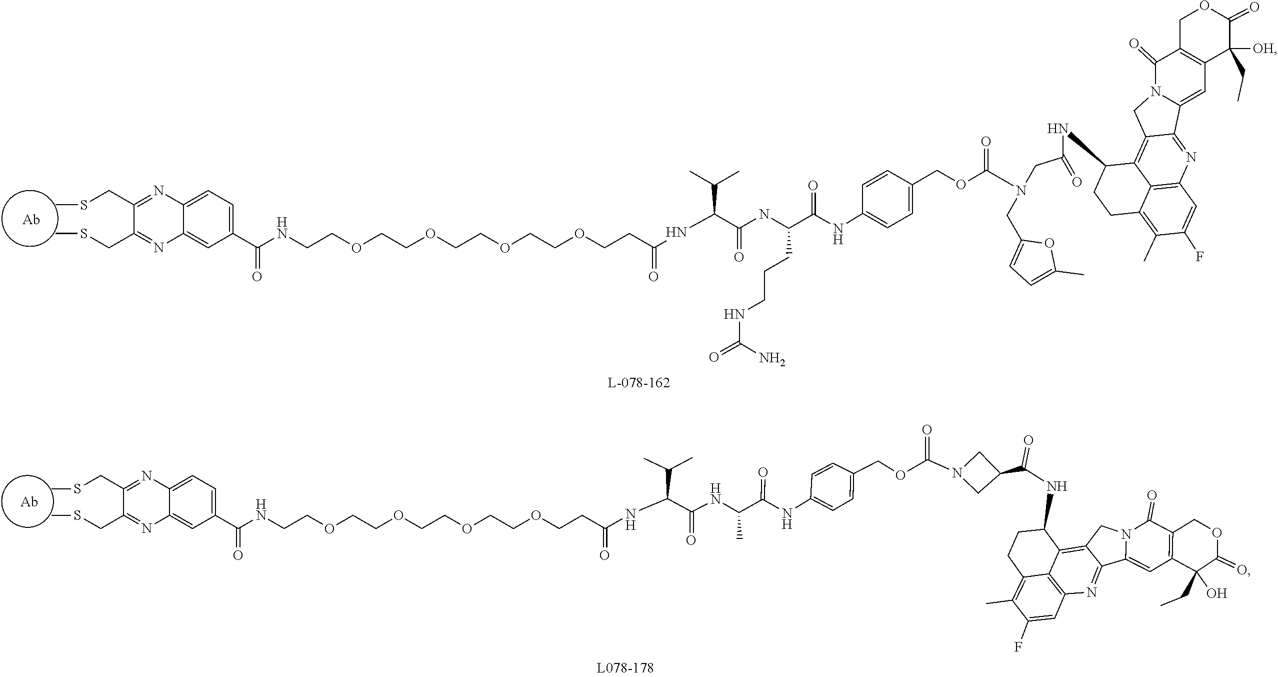 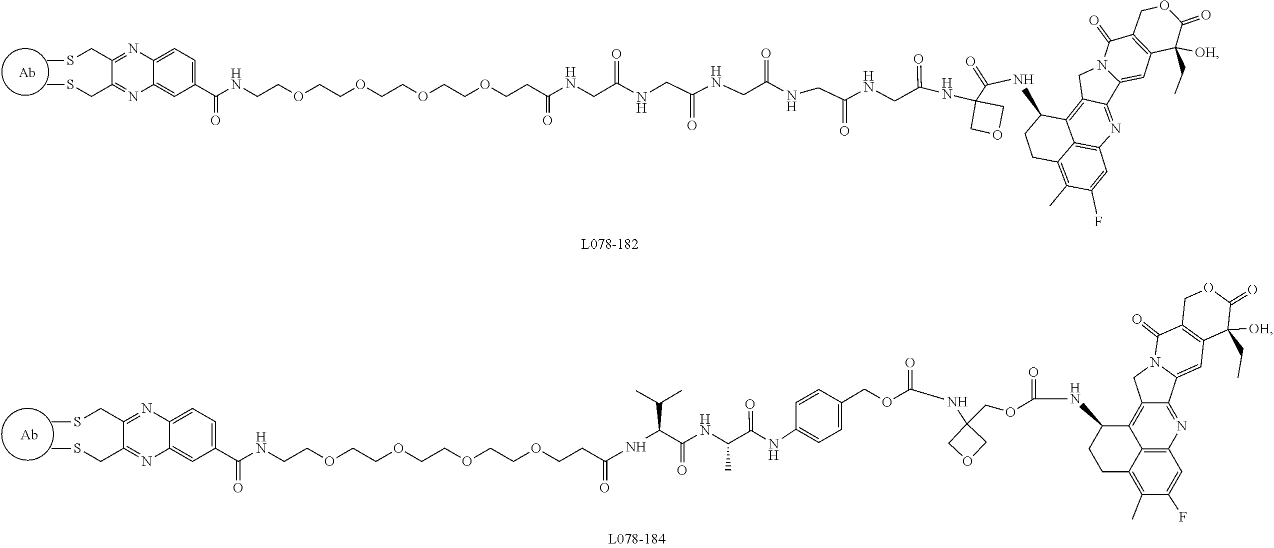 [SMILES: CC[C@]1(O)C(=O)OCc2c1cc1n(c2=O)Cc2c-1nc1cc(F)c(C)c3c1c2[C@H](NC(=O)C1(NC(=O)CNC(=O)CNC(=O)CNC(=O)CNC(=O)CNC(=O)CCOCCOCCOCCOCCNC(=O)c2ccc4nc(CSC)c(CSC)nc4c2)COC1)CC3 ; CC[C@]1(O)C(=O)OCc2c1cc1n(c2=O)Cc2c-1nc1cc(F)c(C)c3c1c2[C@H](NC(=O)OCC1(NC(=O)OCc2ccc(NC(=O)[C@H](C)NC(=O)[C@@H](NC(=O)CCOCCOCCOCCOCCNC(=O)c4ccc5nc(CSC)c(CSC)nc5c4)C(C)C)cc2)COC1)CC3] 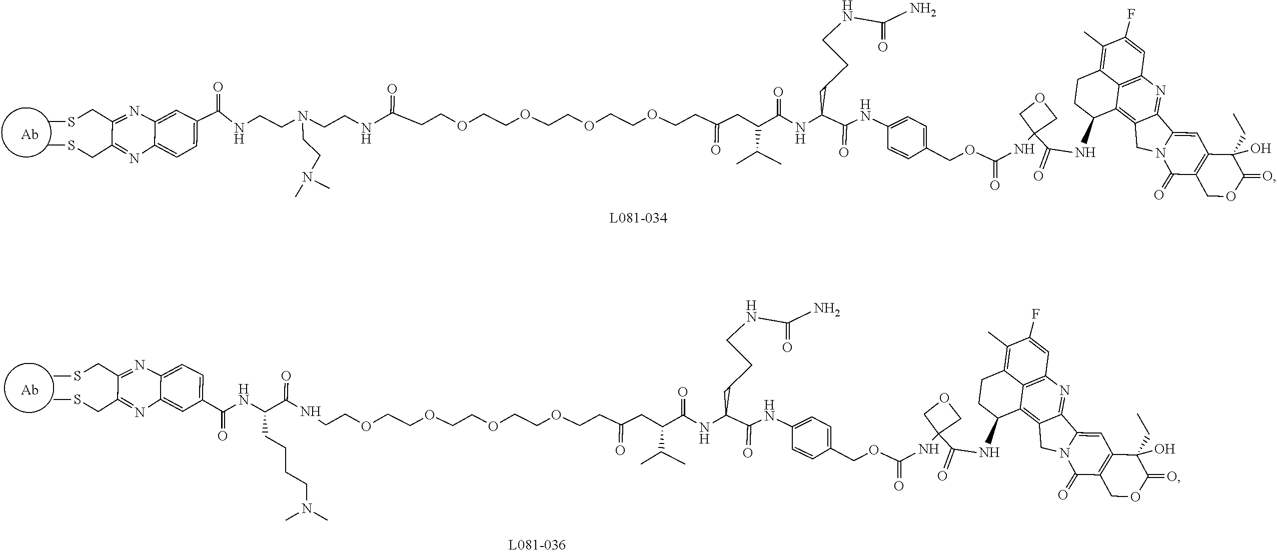 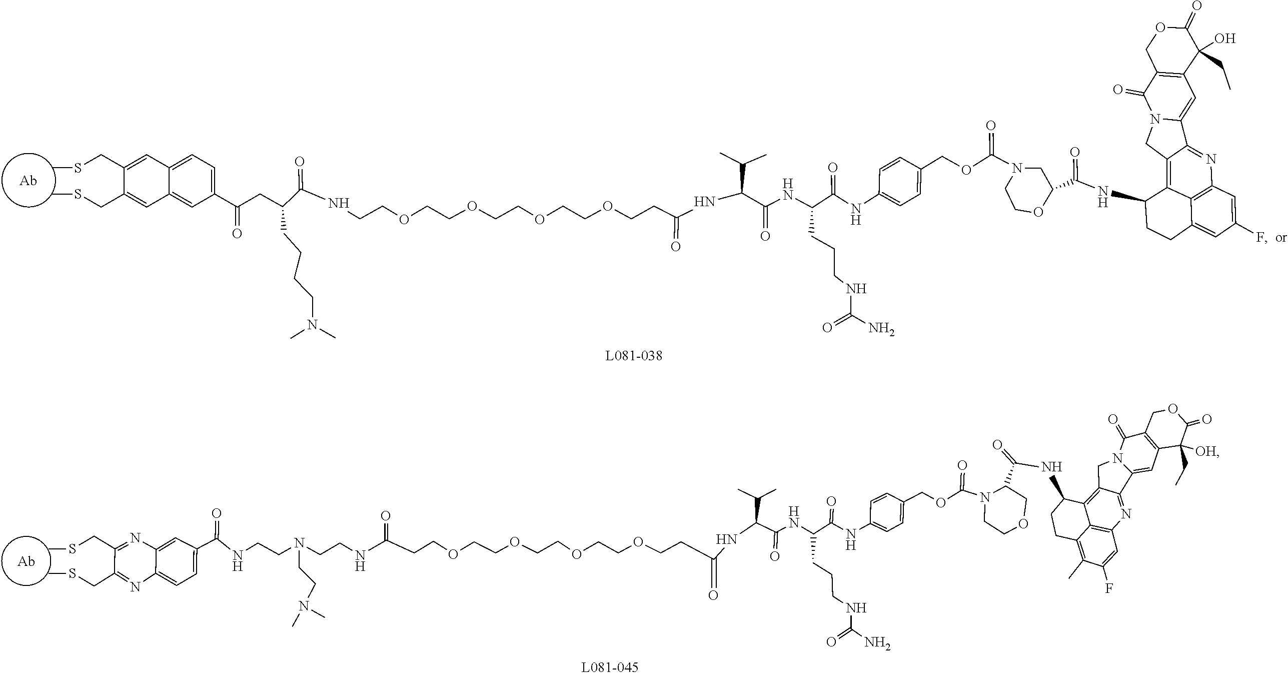
or a pharmaceutically acceptable salt thereof.

54. The ADC of any one of claims 1-53, wherein the anti-HER2 antibody comprises a VL CDR1 comprising the sequence of SEQ ID NO: 1, a VL CDR2 comprising the sequence of SEQ ID NO: 2, a VL CDR3 comprising the sequence of SEQ ID NO: 3, a VH CDR1 comprising the sequence of SEQ ID NO: 4, a VH CDR2 comprising the sequence of SEQ ID NO: 5, and a VH CDR3 comprising the sequence of SEQ ID NO: 6.

55. The ADC of any one of claims 1-54, wherein the anti-HER2 antibody comprises a VL having a sequence with at least 95%, 96%, 97%, 98%, or 99% identity to SEQ ID NO: 7.

56. The ADC of any one of claims 1-55, wherein the anti-HER2 antibody comprises a VH having a sequence with at least 95%, 96%, 97%, 98%, or 99% identity to SEQ ID NO: 8.

57. The ADC of any one of claims 1-56, wherein the anti-HER2 antibody comprises a VL having the sequence of SEQ ID NO: 7.

58. The ADC of any one of claims 1-57, wherein the anti-HER2 antibody comprises a VH having the sequence of SEQ ID NO: 8.

59. The ADC of any one of claims 1-58, wherein the anti-HER2 antibody is an IgG antibody, optionally wherein the anti-HER2 antibody is an IgG1 antibody.

60. The ADC of any one of claims 1-59, wherein the anti-HER2 antibody binds a human HER2, optionally wherein the human HER2 has the amino acid sequence of SEQ ID NO: 16.

61. The ADC or pharmaceutically acceptable salt thereof of any one of claims 1-60, for use in therapy.

62. The ADC or pharmaceutically acceptable salt thereof of claim 61, for use in treating a HER2-expressing cancer.

63. A method of treating a HER2-expressing cancer in a subject, comprising administering the ADC or pharmaceutically acceptable salt thereof of any one of claims 1-60 to a subject in need thereof.

64. Use of the ADC or pharmaceutically acceptable salt thereof of any one of claims 1-60 for the manufacture of a medicament.

65. Use of the ADC or pharmaceutically acceptable salt thereof of any one of claims 1-60 for the manufacture of a medicament for treating a HER2-expressing cancer.

66. The ADC or a pharmaceutically acceptable salt thereof for use, use, or method of any one of claims 62, 63, or 65, wherein the HER2-expressing cancer is breast cancer, lung cancer, ovarian cancer or gastric cancer.

67. The ADC or a pharmaceutically acceptable salt thereof for use, use, or method of claim 66, wherein the breast cancer is metastatic breast cancer or triple negative breast cancer.

68. The ADC or a pharmaceutically acceptable salt thereof for use, use, or method of claim 66, wherein the lung cancer is non-small cell lung cancer (NSCLC).

69. A method of preparing the ADC of any one of claims 1-53, comprising reacting a monoclonal antibody with a compound of formula (P-I) or formula (P-II):
B-L²-L³-D (P-I)
or
B-L²-D′-R¹ (P-II)
or a pharmaceutically acceptable salt thereof, wherein:
B is a reactive moiety capable of forming a bond with the monoclonal antibody;
L² is a bond, —C(O)—, —NH—, Amino Acid Unit, —(CH₂CH₂O)ₙ—, —(CH₂)ₙ—, —O—-(4-aminobenzyloxycarbonyl)-, —(C(O)CH₂CH₂NH)—, —(C(O)N(R²)CH₂CH₂N(R³))—, or any combination thereof, wherein n is an integer from 1 to 24;
each R² and R³ is independently H or substituted or unsubstituted alkyl;
L³ is a substituted or unsubstituted heterocycloalkylene, substituted or unsubstituted heteroarylene substituted or unsubstituted heterocycloalkyl, or substituted or unsubstituted heteroaryl; or L³ is substituted or unsubstituted —OCH₂-(heterocycloalkyl) or substituted or unsubstituted —OCH₂-(heteroaryl), wherein L³ is linked to D through oxygen; or L³ is substituted or unsubstituted —CH₂NCH₂-(heteroaryl) or substituted or unsubstituted —CH₂NCH₂-(heterocycloalkyl), wherein L³ is linked to D through —CH₂—, and through nitrogen to L²;
R¹ is a substituted or unsubstituted heterocycloalkyl or a substituted or unsubstituted heteroaryl;
D is 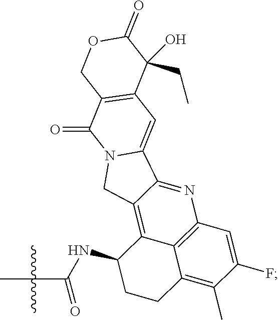 [SMILES: *C(=O)N[C@@H]1CCc2c(C)c(F)cc3nc4c(c1c23)Cn1c-4cc2c(c1=O)COC(=O)[C@@]2(O)CC]
and
D′ is 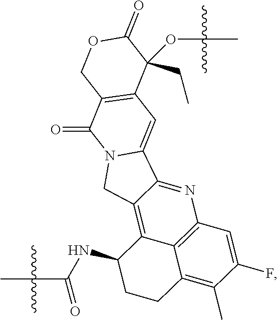
wherein D′ is connected through its amide group to R¹, and through oxygen to L².

70. The method of claim 69, wherein the monoclonal antibody is an anti-HER2 antibody.

71. The method of claim 69 or 70, wherein the monoclonal antibody is modified with an aldehyde, azide, alkyne, tetrazine, hydrazine, alkoxyamine, trans-cyclooctene or cyclopropene.

72. The method of any one of claims 69-71, wherein B is a reactive moiety capable of forming a bond with one or two thiol or amine groups of the monoclonal antibody, or with the modified monoclonal antibody.

73. The method of claim 72, wherein B is:

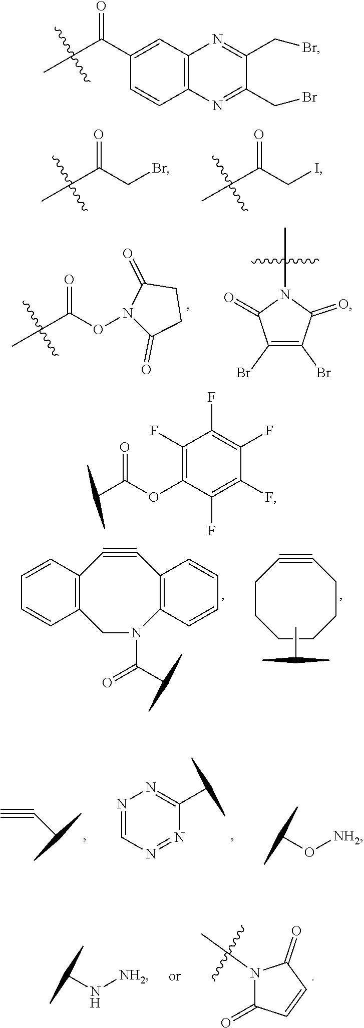

74. The method of any one of claims 69-73, wherein L² is a bond, —C(O)—, —NH—, -Val-, -Phe-, -Lys-, -(4-aminobenzyloxycarbonyl)-, -Gly-, -Ser-, -Thr-, -Ala-, -β-Ala-, -citrulline- (-Cit-), —O—, —(CH₂)ₙ—, —(CH₂CH₂O)ₙ—, —(C(O)N(CH₃)CH₂CH₂N(CH₃))—, or any combination thereof.

75. The method of claim 74, wherein L² is —C(O)—, —NH—, -Val-, -Ala-, -Gly-, -Cit-, —O—, -(4-aminobenzyloxycarbonyl)-, —(CH₂)ₙ—, —(CH₂CH₂O)ₙ—, —(C(O)N(CH₃)CH₂CH₂N(CH₃))—, or any combination thereof.

76. The method of claim 74, wherein L² is —C(O)—, —NH—, -Gly-, —(CH₂)ₙ—, —(CH₂CH₂O)ₙ—, or any combination thereof.

77. The method of claim 74, wherein L² is:

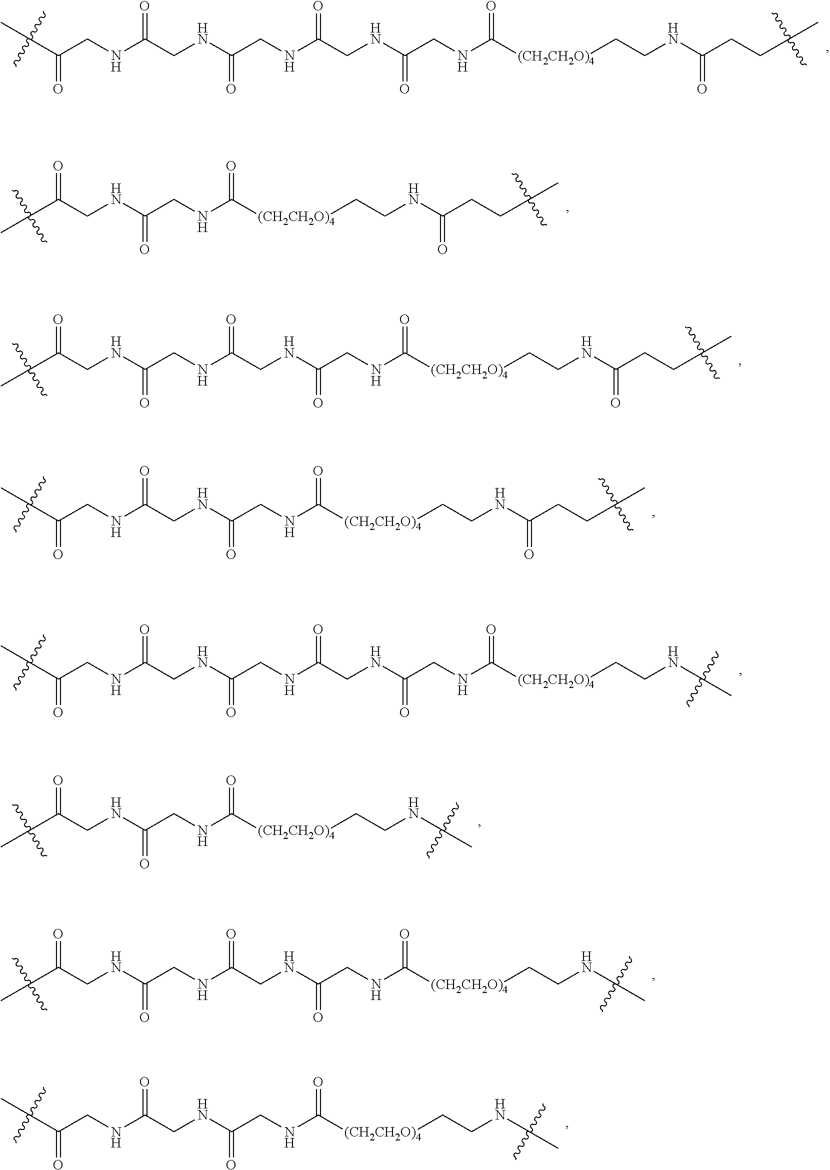

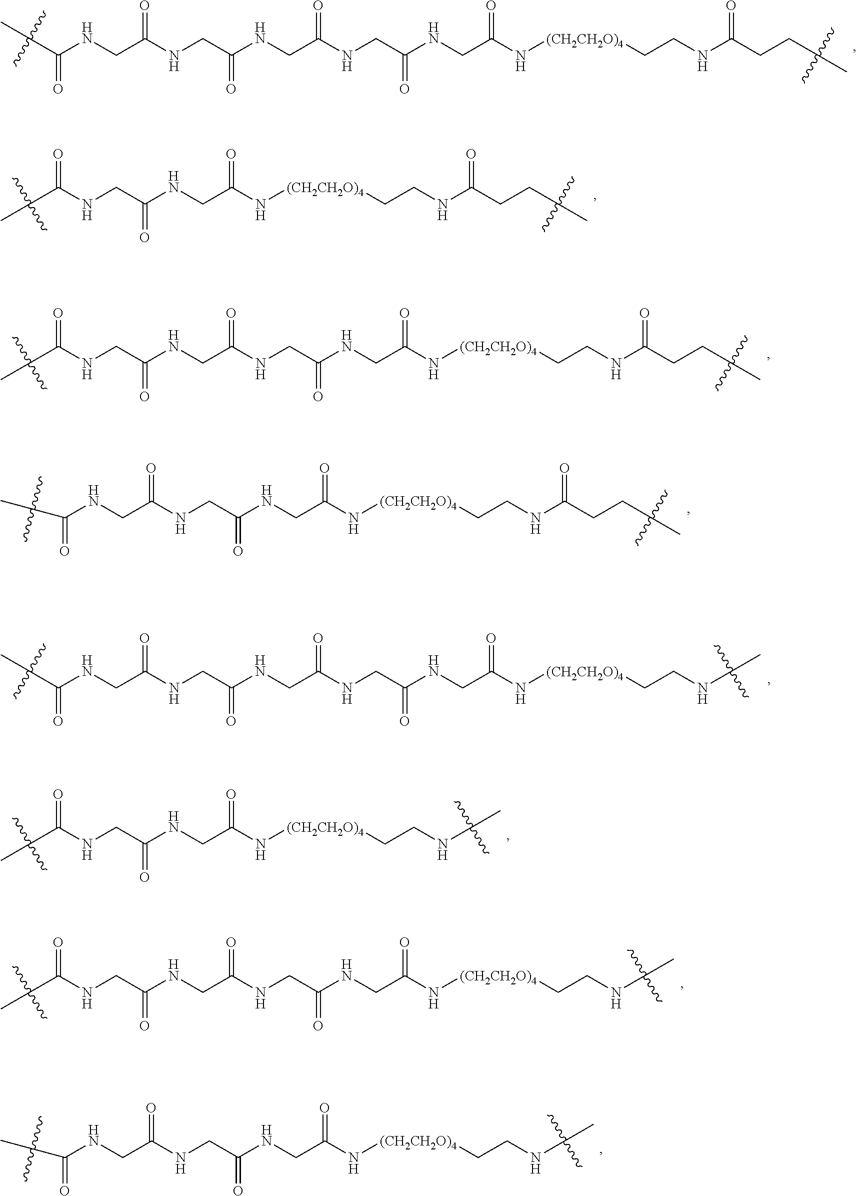

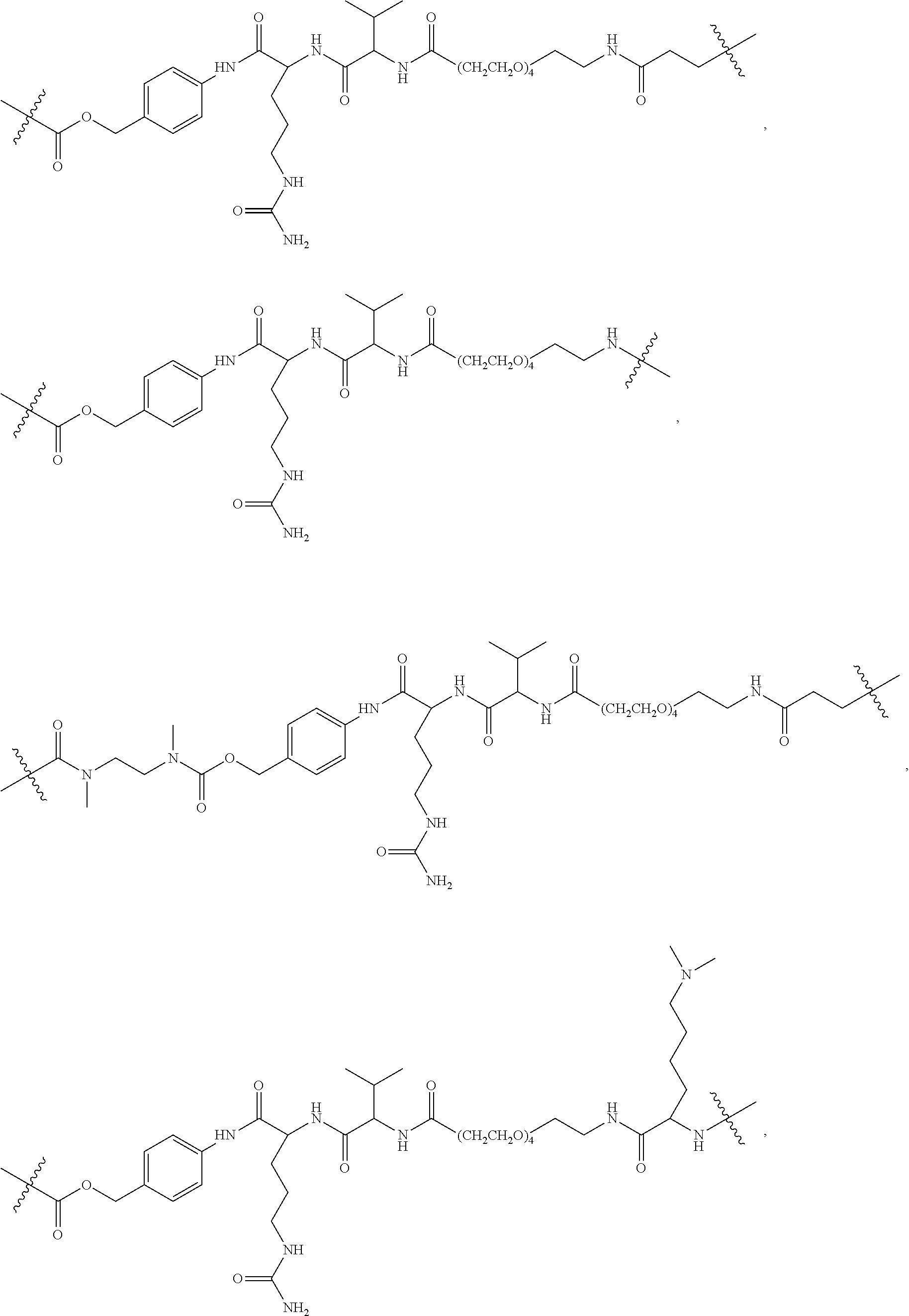

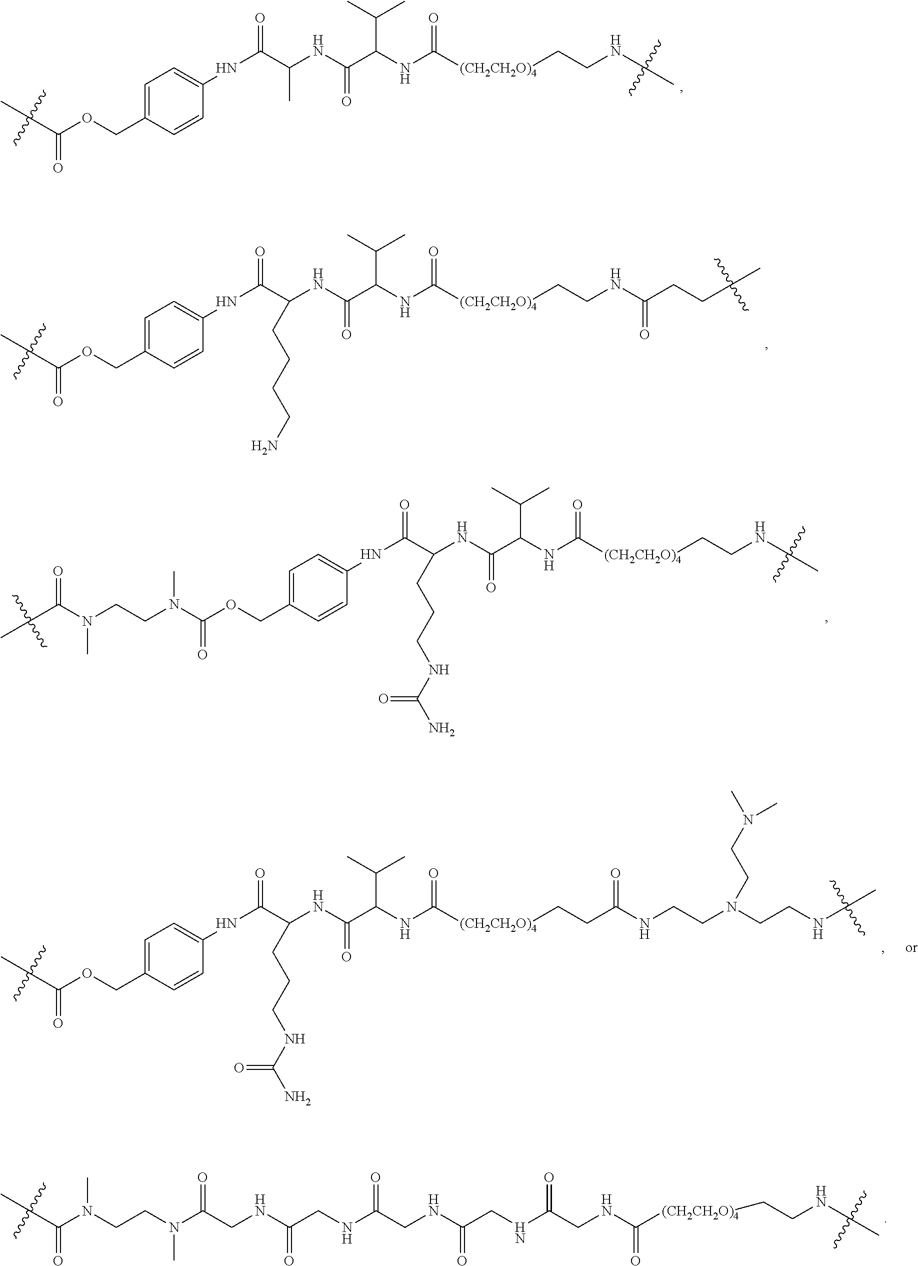

78. The method of claim 77, wherein L² is

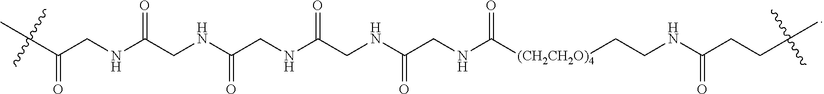

79. The method of claim 77, wherein L² is

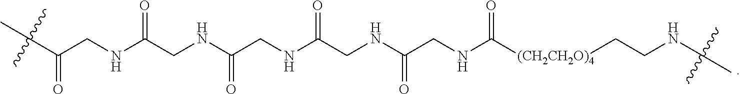

80. The method of claim 77, wherein L² is

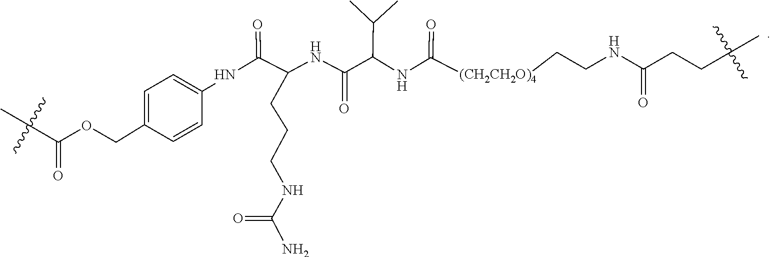

81. The method of claim 77, wherein L² is

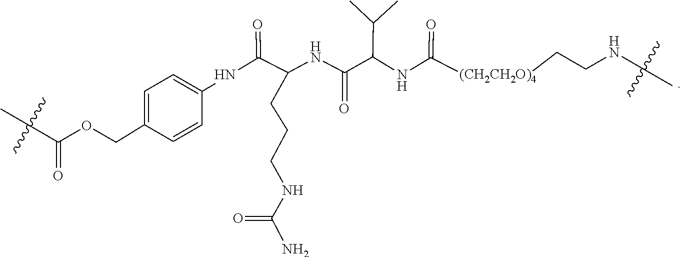

82. The method of claim 77, wherein L² is

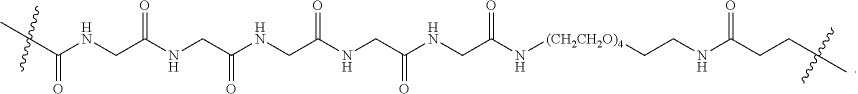

83. The method of claim 77, wherein L² is

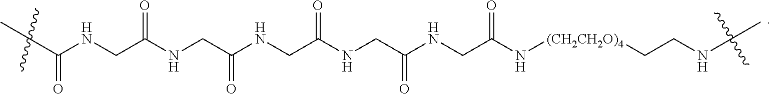

84. The method of claim 77, wherein L² is

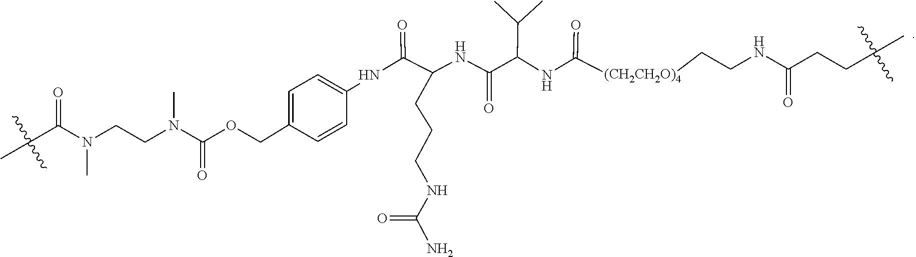

85. The method of claim 77, wherein L² is

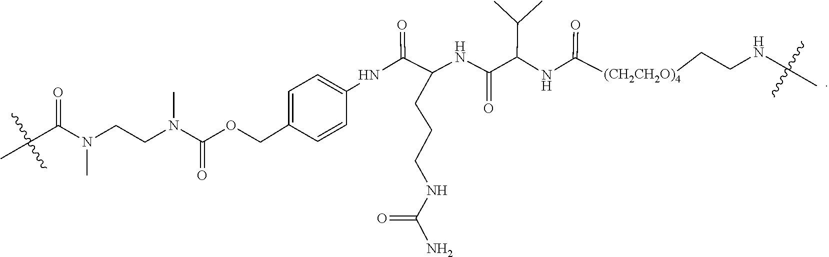

86. The ADC or a pharmaceutically acceptable salt thereof of claim 77, wherein L² is

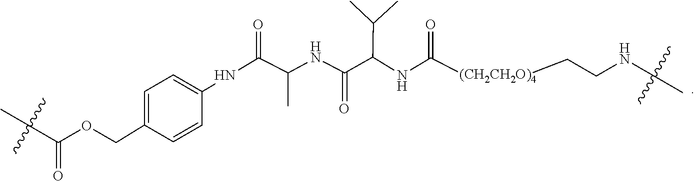

87. The ADC or a pharmaceutically acceptable salt thereof of claim 77, wherein L² is

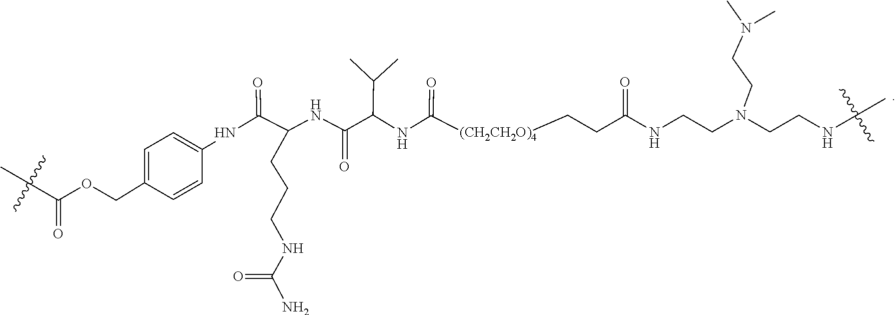

88. The ADC or a pharmaceutically acceptable salt thereof of claim 77, wherein L² is

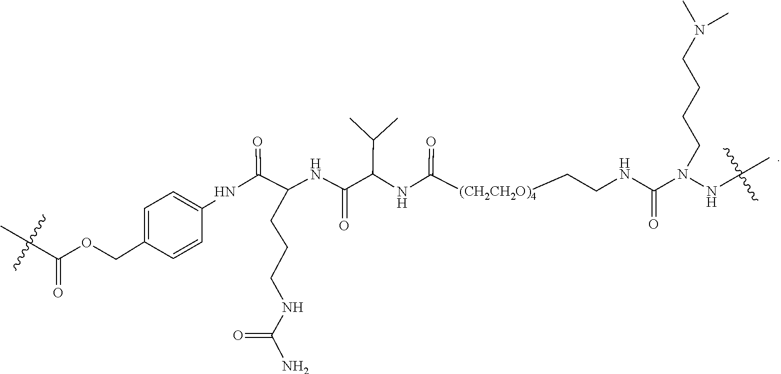

89. The ADC or a pharmaceutically acceptable salt thereof of claim 77, wherein L² is

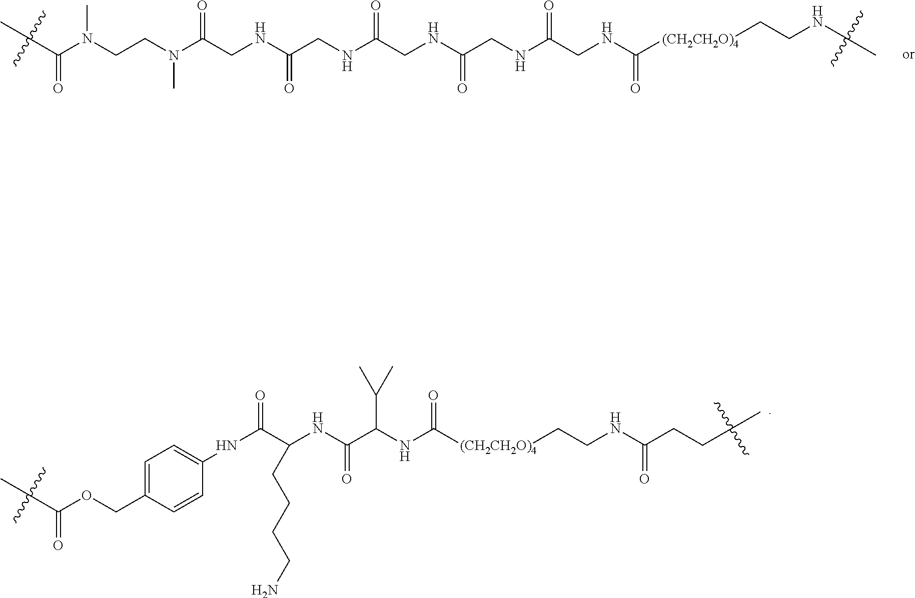

90. The method of any one of claims 69-89, wherein L³ is a substituted or unsubstituted heterocycloalkylene; or substituted or unsubstituted heterocycloalkyl; or L³ is substituted or unsubstituted —OCH₂-(heterocycloalkyl), wherein L³ is linked to D through oxygen; or L³ is substituted or unsubstituted —CH₂NCH₂-(heterocycloalkyl), wherein L³ is linked to D through —CH₂—, and through nitrogen to L².

91. The method of claim 90, wherein L³ is a substituted or unsubstituted 3 to 8 membered heterocycloalkylene; or substituted or unsubstituted 3 to 8 membered heterocycloalkyl; or L³ is substituted or unsubstituted —OCH₂-(3 to 8 membered heterocycloalkyl), wherein L³ is linked to D through oxygen; or L³ is substituted or unsubstituted —CH₂NCH₂-(3 to 8 membered heterocycloalkyl), wherein L³ is linked to D through —CH₂—, and through nitrogen to L².

92. The method of claim 90, wherein L³ is a substituted or unsubstituted 3 to 6 membered heterocycloalkylene; or substituted or unsubstituted 3 to 6 membered heterocycloalkyl; or L³ is substituted or unsubstituted —OCH₂-(3 to 6 membered heterocycloalkyl), wherein L³ is linked to D through oxygen; or L³ is substituted or unsubstituted —CH₂NCH₂-(3 to 6 membered heterocycloalkyl), wherein L³ is linked to D through —CH₂—, and through nitrogen to L².

93. The method of claim 92, wherein L³ is a substituted or unsubstituted heterocyclobutylene, heterocyclopentylene or heterocyclohexylene; or substituted or unsubstituted heterocyclobutyl, heterocyclopentyl, or heterocyclohexyl; or L³ is substituted or unsubstituted —OCH₂-(heterocyclobutyl, heterocyclopentyl, or heterocyclohexyl), wherein L³ is linked to D through oxygen; or L³ is substituted or unsubstituted —CH₂NCH₂-(heterocyclobutyl, heterocyclopentyl, or heterocyclohexyl), wherein L³ is linked to D through —CH₂—, and through nitrogen to L².

94. The method of any one of claims 69-89, wherein L³ is a substituted or unsubstituted heteroarylene or substituted or unsubstituted heteroaryl; or L³ is substituted or unsubstituted —OCH₂-(heteroaryl), wherein L³ is linked to D through oxygen; or substituted or unsubstituted —CH₂NCH₂-(heteroaryl), wherein L³ is linked to D through —CH₂—, and through nitrogen to L.

95. The method of claim 94, wherein L³ is a substituted or unsubstituted 5 to 10 membered heteroarylene; or substituted or unsubstituted 5 to 10 membered heteroaryl; or L³ is substituted or unsubstituted —OCH₂-(5 to 10 membered heteroaryl), wherein L³ is linked to D through oxygen; or L³ is substituted or unsubstituted —CH₂NCH₂-(5 to 10 membered heteroaryl), wherein L³ is linked to D through —CH₂—, and through nitrogen to L².

96. The method of claim 94, wherein L³ is a substituted or unsubstituted 5 to 9 membered heteroarylene; or substituted or unsubstituted 5 to 9 membered heteroaryl; or L³ is substituted or unsubstituted —OCH₂-(5 to 9 membered heteroaryl), wherein L³ is linked to D through oxygen; or L³ is substituted or unsubstituted —CH₂NCH₂-(5 to 9 membered heteroaryl), wherein L³ is linked to D through —CH₂—, and through nitrogen to L².

97. The method of claim 94, wherein L³ is a substituted or unsubstituted 5 to 6 membered heteroarylene; or substituted or unsubstituted 5 to 6 membered heteroaryl; or L³ is substituted or unsubstituted —OCH₂-(5 to 6 membered heteroaryl), wherein L³ is linked to D through oxygen; or L³ is substituted or unsubstituted —CH₂NCH₂-(5 to 6 membered heteroaryl), wherein L³ is linked to D through —CH₂—, and through nitrogen to L².

98. The method of claim 97, wherein L³ is a substituted or unsubstituted furanylene, pyrrolylene, pyridylene, pyranylene, imidazolylene, thienylene, oxazolylene, or thiazolylene; or substituted or unsubstituted furanyl, pyrrolyl, pyridyl, pyranyl, imidazolyl, thiazolyl, thienyl, or oxazolyl; or L³ is substituted or unsubstituted —OCH₂-(furanyl, pyrrolyl, pyridyl, pyranyl, imidazolyl, thiazolyl, thienyl, or oxazolyl), wherein L³ is linked to D through oxygen; or L³ is substituted or unsubstituted —CH₂NCH₂-(furanyl, pyrrolyl, pyridyl, pyranyl, imidazolyl, thiazolyl, thienyl, or oxazolyl), wherein L³ is linked to D through —CH₂—, and through nitrogen to L².

99. The method of any one of claims 69-85, wherein R¹ is a substituted or unsubstituted heterocycloalkyl.

100. The method of claim 99, wherein R¹ is a substituted or unsubstituted 3 to 8 membered heterocycloalkyl.

101. The method of claim 99, wherein R¹ is a substituted or unsubstituted 3 to 6 membered heterocycloalkyl.

102. The method of claim 101, wherein R¹ is a substituted or unsubstituted heterocyclobutyl, heterocyclopentyl or heterocyclohexyl.

103. The method of any one of claims 69-85, wherein R¹ is a substituted or unsubstituted heteroaryl.

104. The method of claim 103, wherein R¹ is a substituted or unsubstituted 5 to 10 membered heteroaryl.

105. The method of claim 103, wherein R¹ is a substituted or unsubstituted 5 to 9 membered heteroaryl.

106. The method of claim 103, wherein R¹ is a substituted or unsubstituted 5 to 6 membered heteroaryl.

107. The method of claim 103, wherein R¹ is a substituted or unsubstituted furanyl, pyrrolyl, pyridyl, pyranyl, imidazolyl, or thiazolyl.

108. The method of any one of claims 69-89, wherein the compound has the formula (P-IA) or formula (P-IIA): 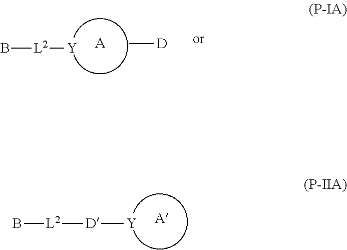
or a pharmaceutically acceptable salt thereof, wherein:
ring A is a substituted or unsubstituted heterocycloalkylene or a substituted or unsubstituted heteroarylene, connected to L² through a heteroatom Y;
ring A′ is a substituted or unsubstituted heterocycloalkyl or a substituted or unsubstituted heteroaryl, connected to D′ through a heteroatom Y; and
each Y is independently N, P, or S.

109. The method of claim 108, wherein the compound has the formula (P-IB) or formula (P-IIB): 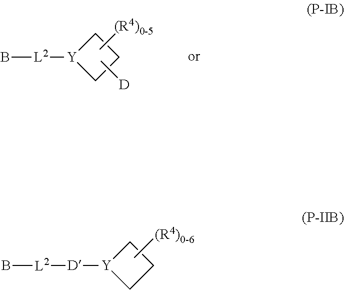
or a pharmaceutically acceptable salt thereof, wherein:
each R⁴ is independently H, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A}, —POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁴ substituents on adjacent carbons may optionally be joined to form a substituted or unsubstituted cycloalkyl, substituted or unsubstituted heterocycloalkyl, substituted or unsubstituted aryl or substituted or unsubstituted heteroaryl;
each R^{4A} and R^{4B} is independently H, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X is —Cl, —Br, —I or —F;
each n4 is independently an integer from 0 to 4;
each v4 is independently 1 or 2;
each m4 is independently an integer from 0 to 3; and
each r4 is independently 1 or 2.

110. The method of claim 108, wherein the compound has the formula (P-IC) or formula (P-IIC): 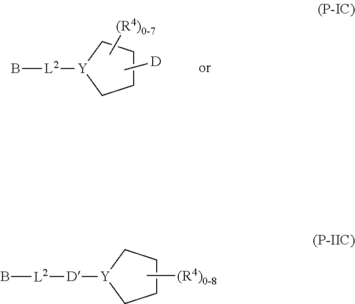
or a pharmaceutically acceptable salt thereof.

111. The method of claim 108, wherein the compound has the formula (P-ID) or formula (P-IID): 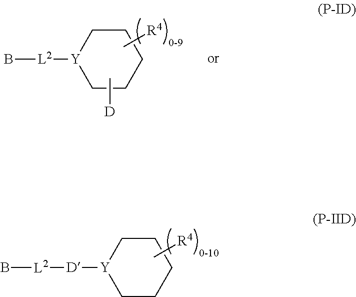
or a pharmaceutically acceptable salt thereof.

112. The method of claim 108, wherein the compound has the formula (P-ID1) or formula (P-IID1): 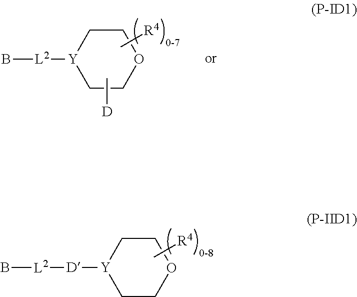
or a pharmaceutically acceptable salt thereof.

113. The method of claim 108, wherein the compound has the formula (P-IE) or formula (P-IIE): 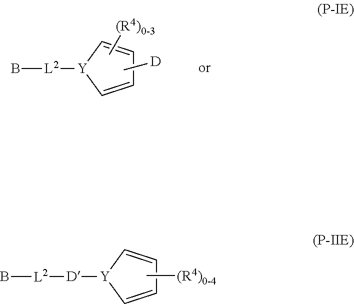
or a pharmaceutically acceptable salt thereof.

114. The method of claim 108, wherein the compound has the formula (P-IF) or formula (P-IIF): 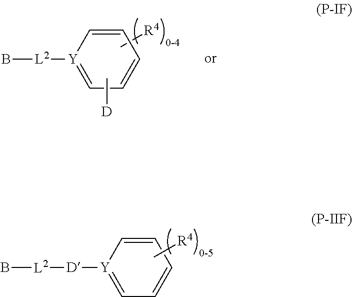
or a pharmaceutically acceptable salt thereof.

115. The method of claim 108, wherein the compound has the formula (P-IG) or formula (P-IH): 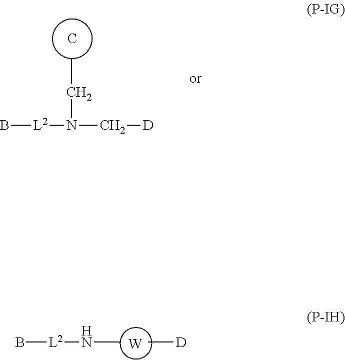
or a pharmaceutically acceptable salt thereof, wherein:
ring W is a substituted or unsubstituted cycloalkylene or a substituted or unsubstituted arylene;
ring C is a substituted or unsubstituted cycloalkyl, substituted or unsubstituted heterocycloalkyl, substituted or unsubstituted aryl, or substituted or unsubstituted heteroaryl.

116. The method of claim 108, wherein the compound has the formula (P-IJ) or formula (P-IK): 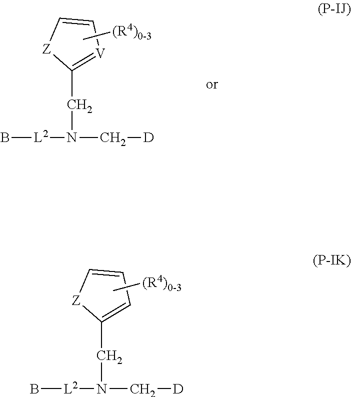
or a pharmaceutically acceptable salt thereof, wherein:
Z is S, N, or O; and
V is C or N.

117. The method of claim 108, wherein the compound has the formula (P-IL) or formula (P-IM): 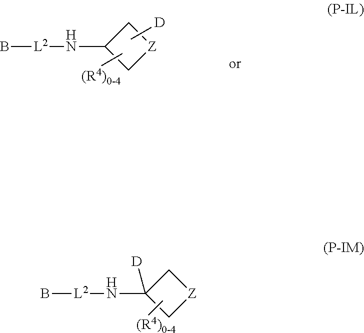
or a pharmaceutically acceptable salt thereof.

118. The method of claim 108, wherein the compound has the formula (P-IN) or formula (P-IO): 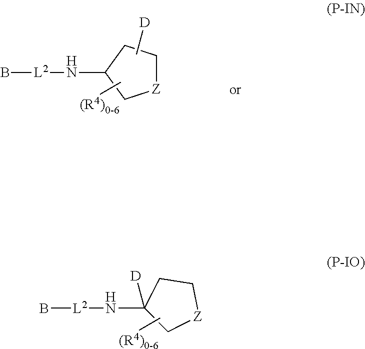
or a pharmaceutically acceptable salt thereof.

119. The method of claim 108, wherein the compound has the formula (P-IP) or formula (P-IQ): 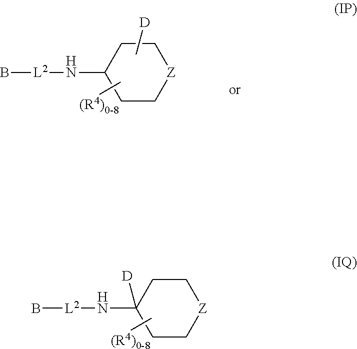
or a pharmaceutically acceptable salt thereof.

120. The method of any one of claims 69-119, wherein B-L²-L³-D is 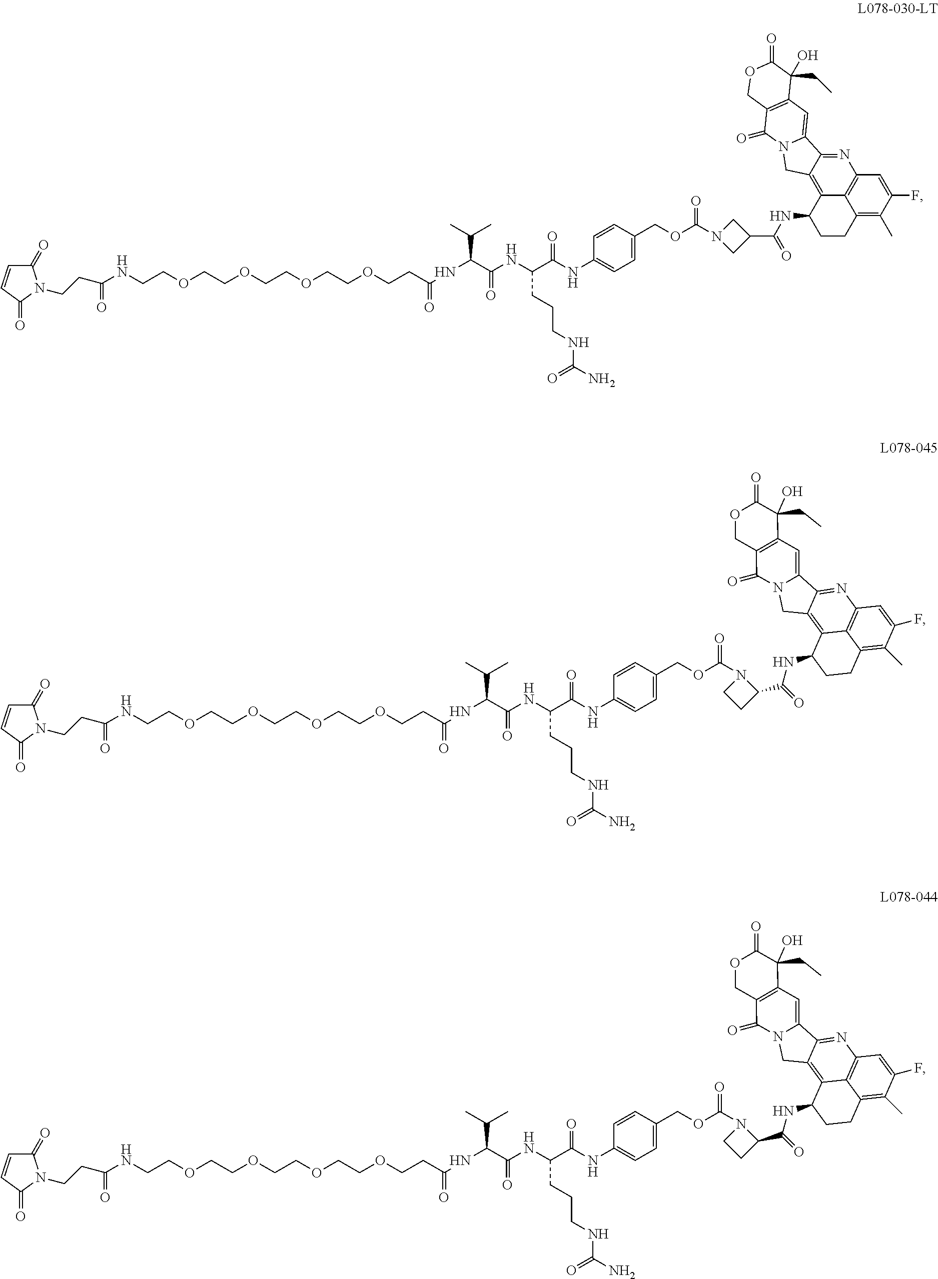 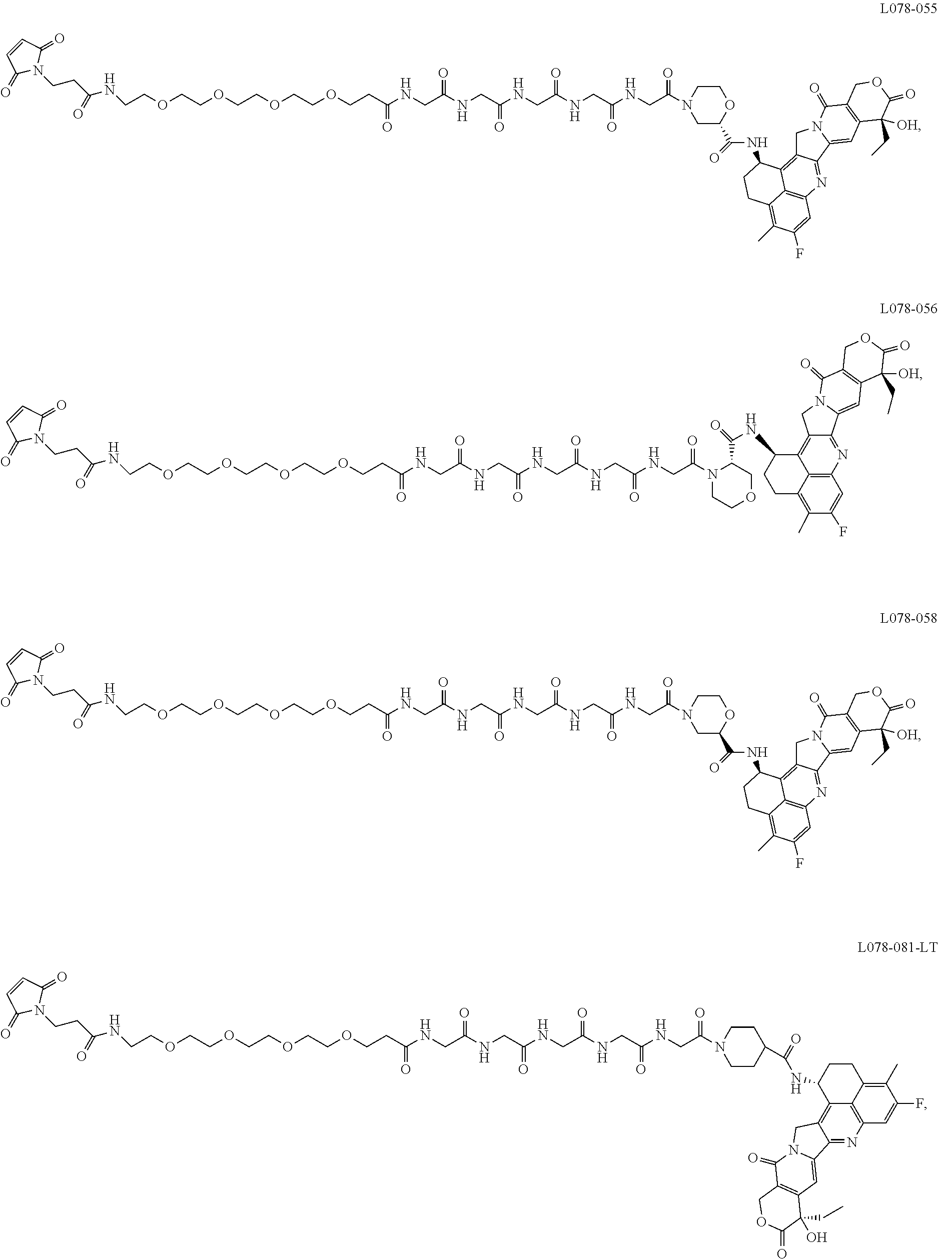 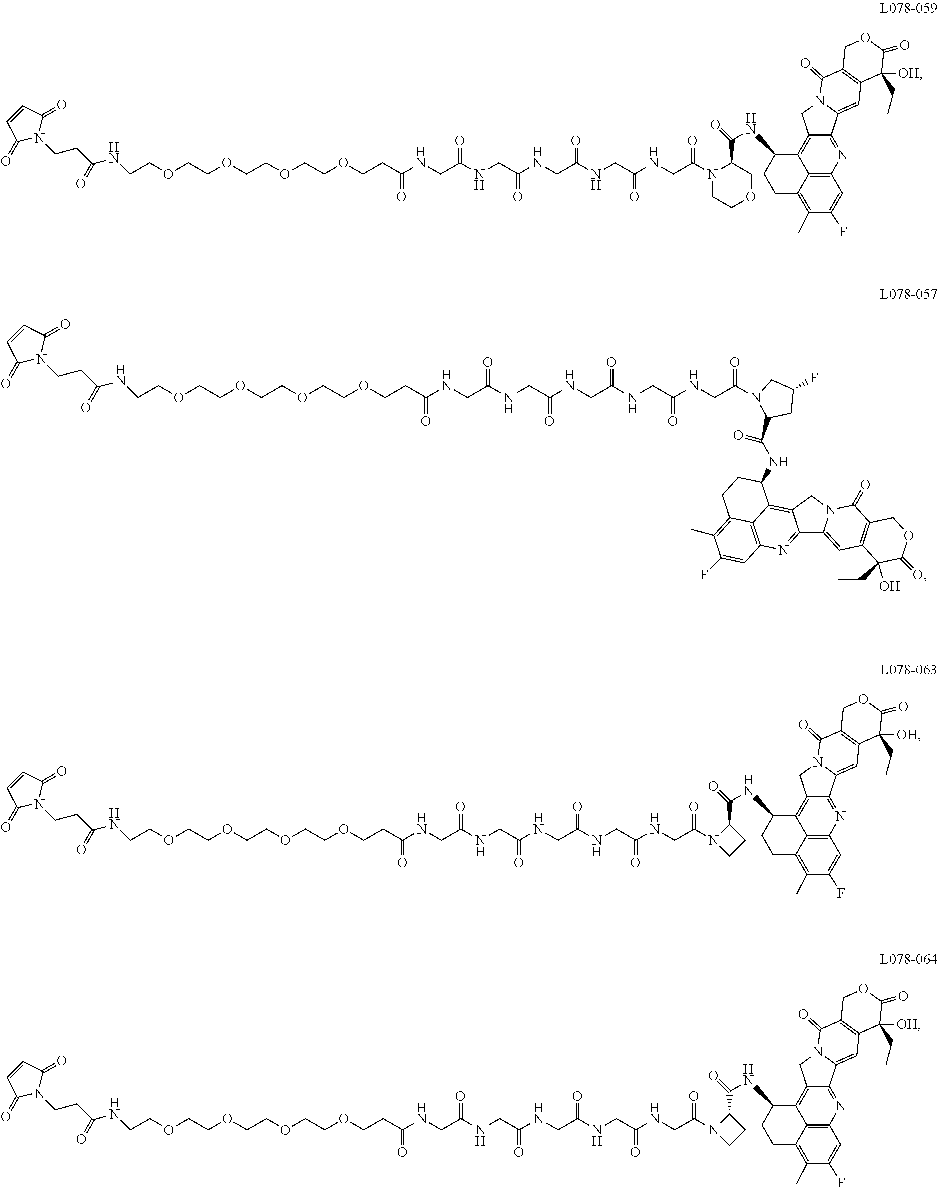 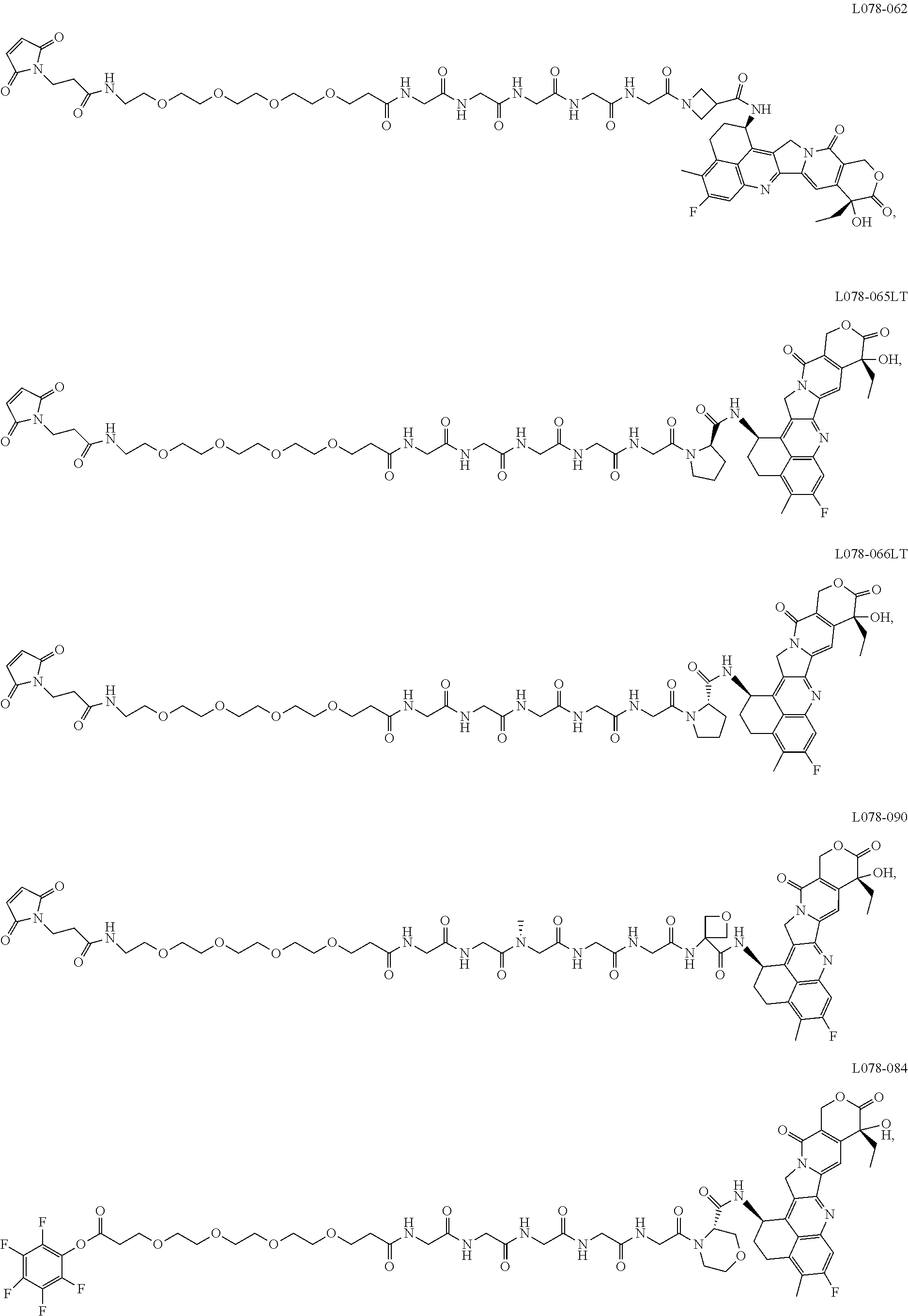 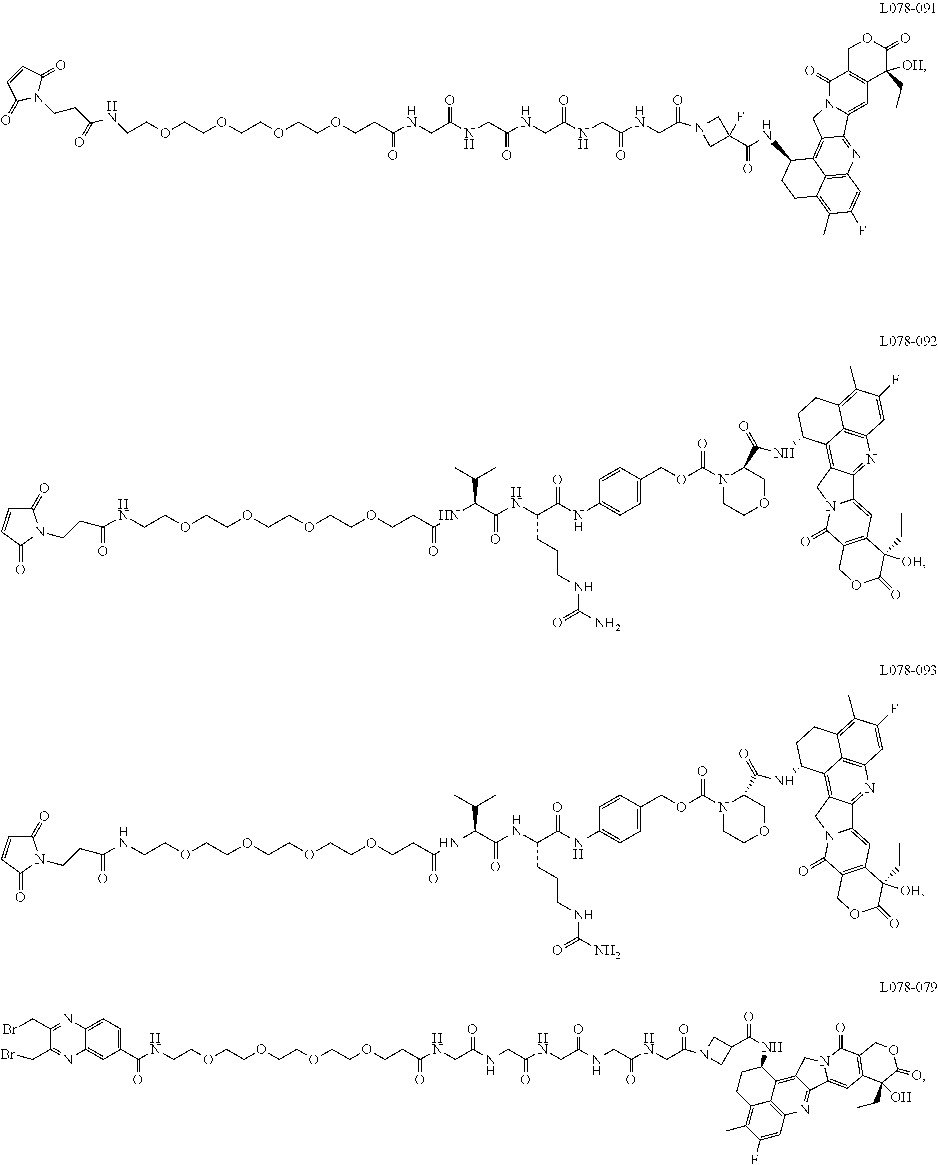 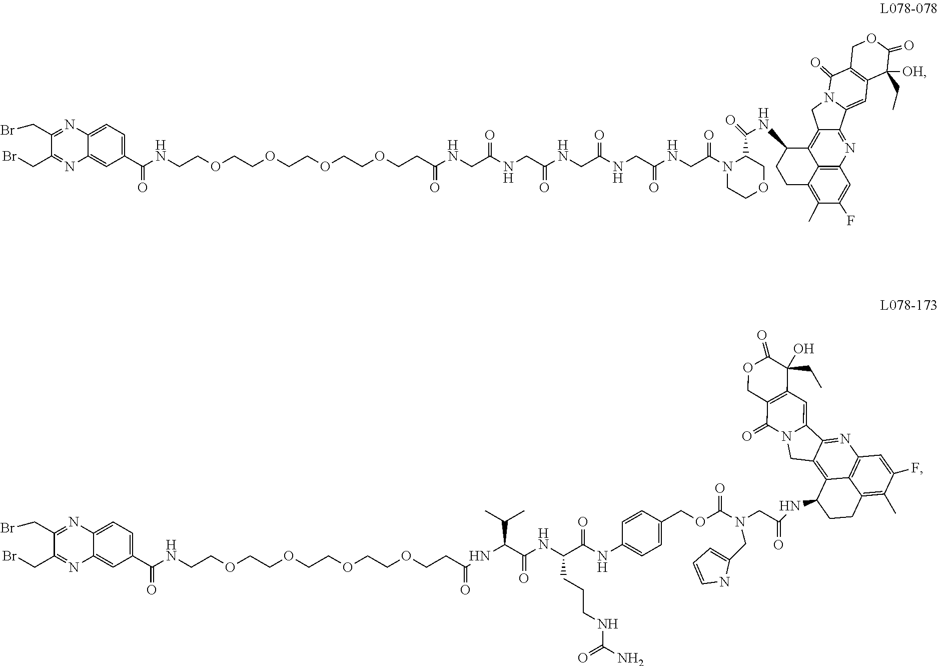 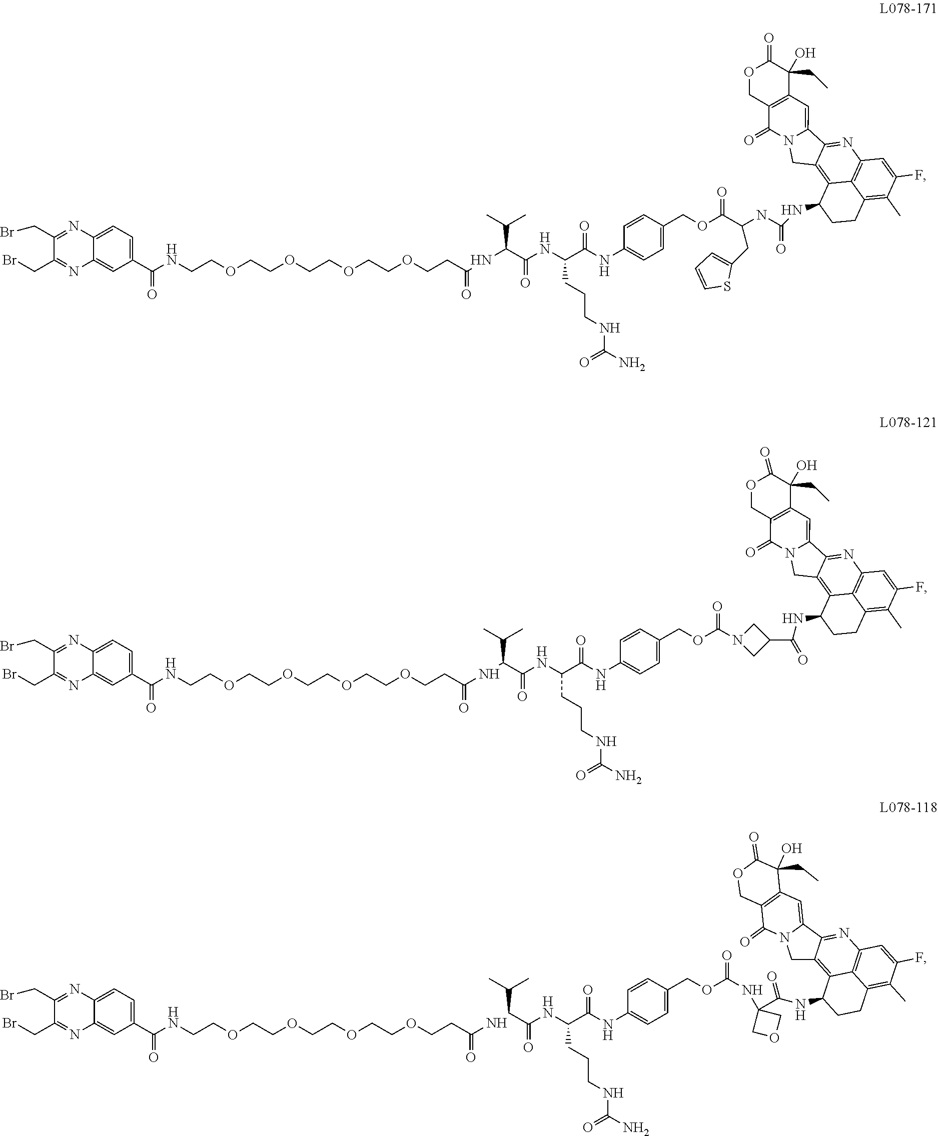 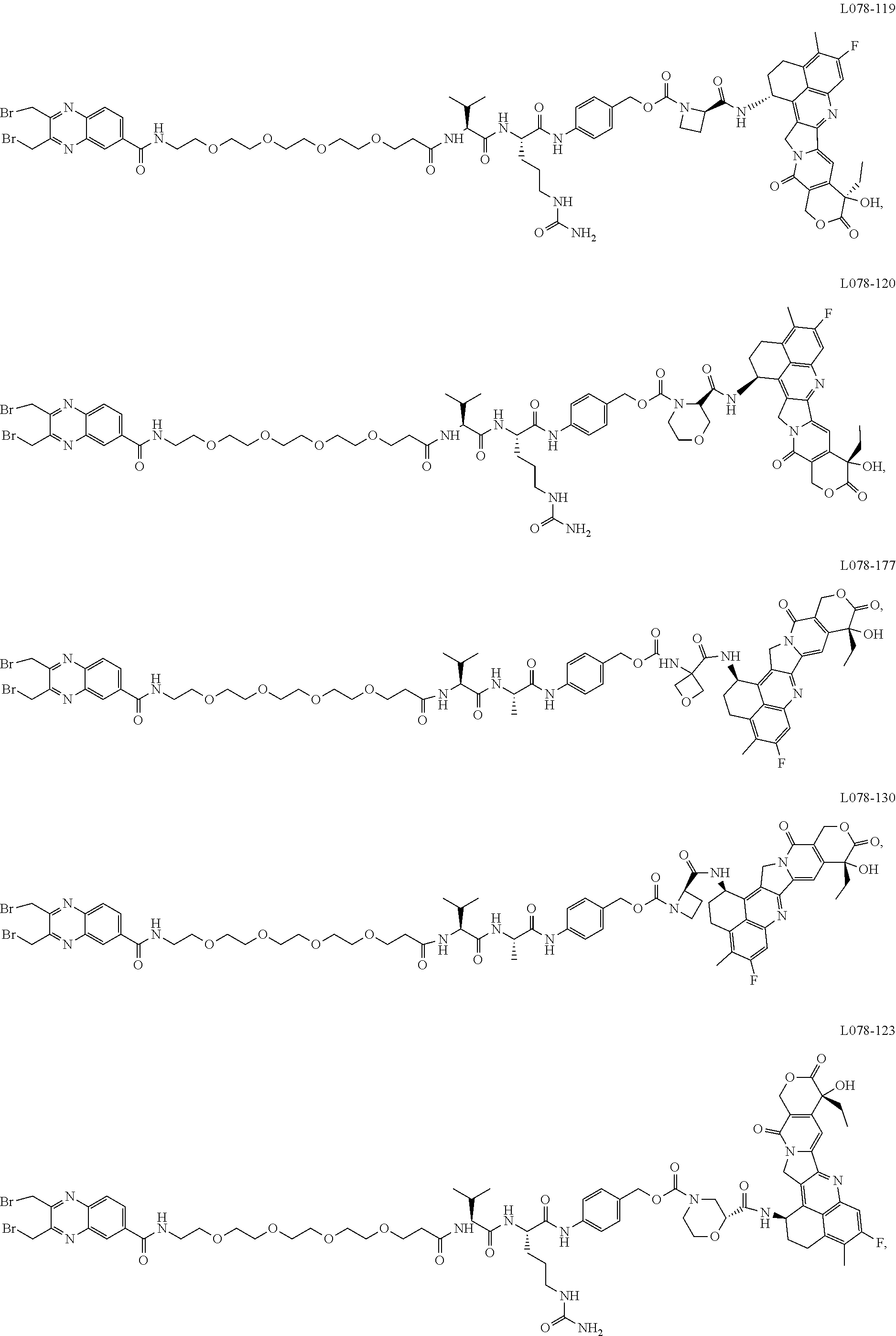 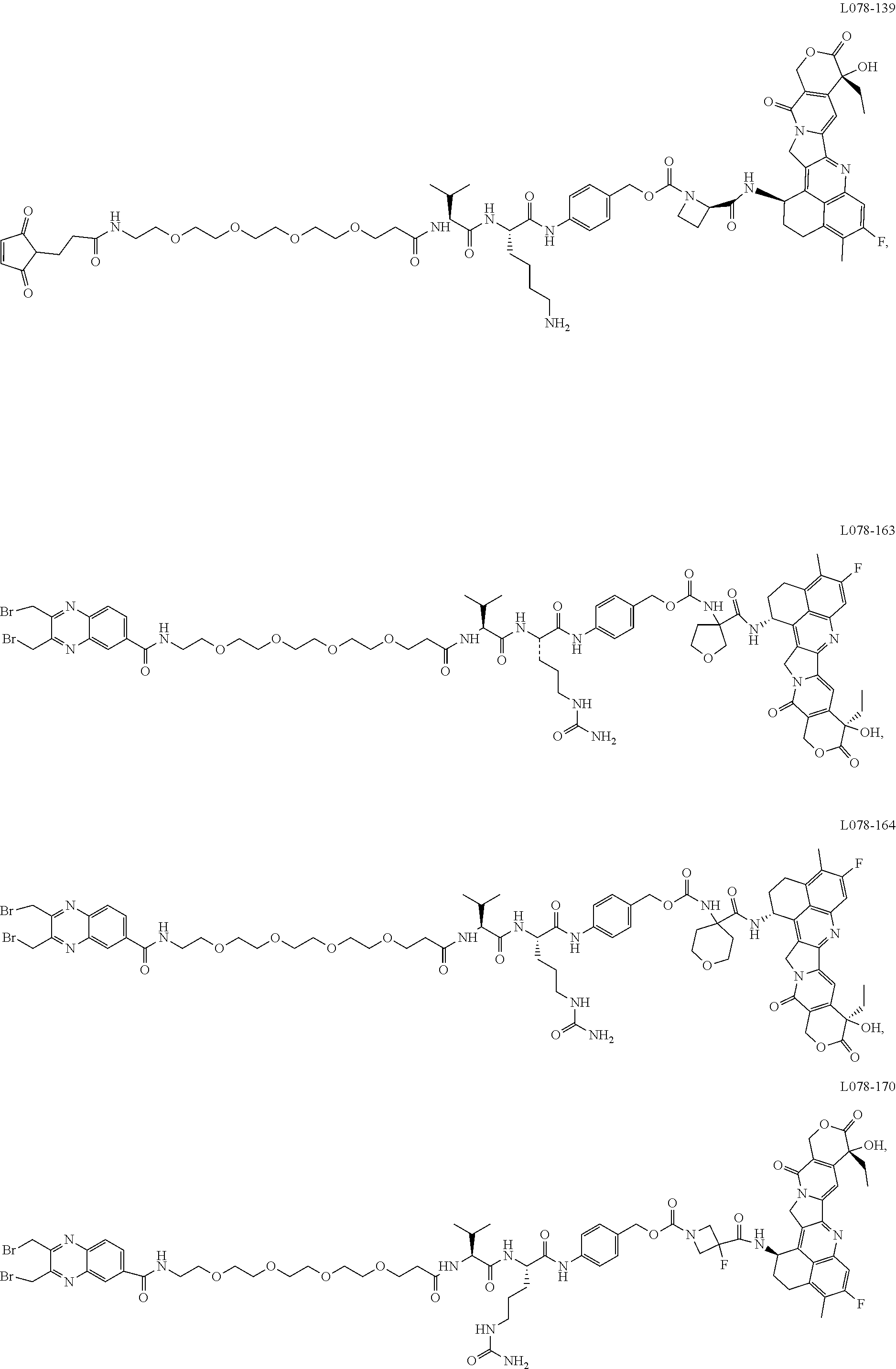 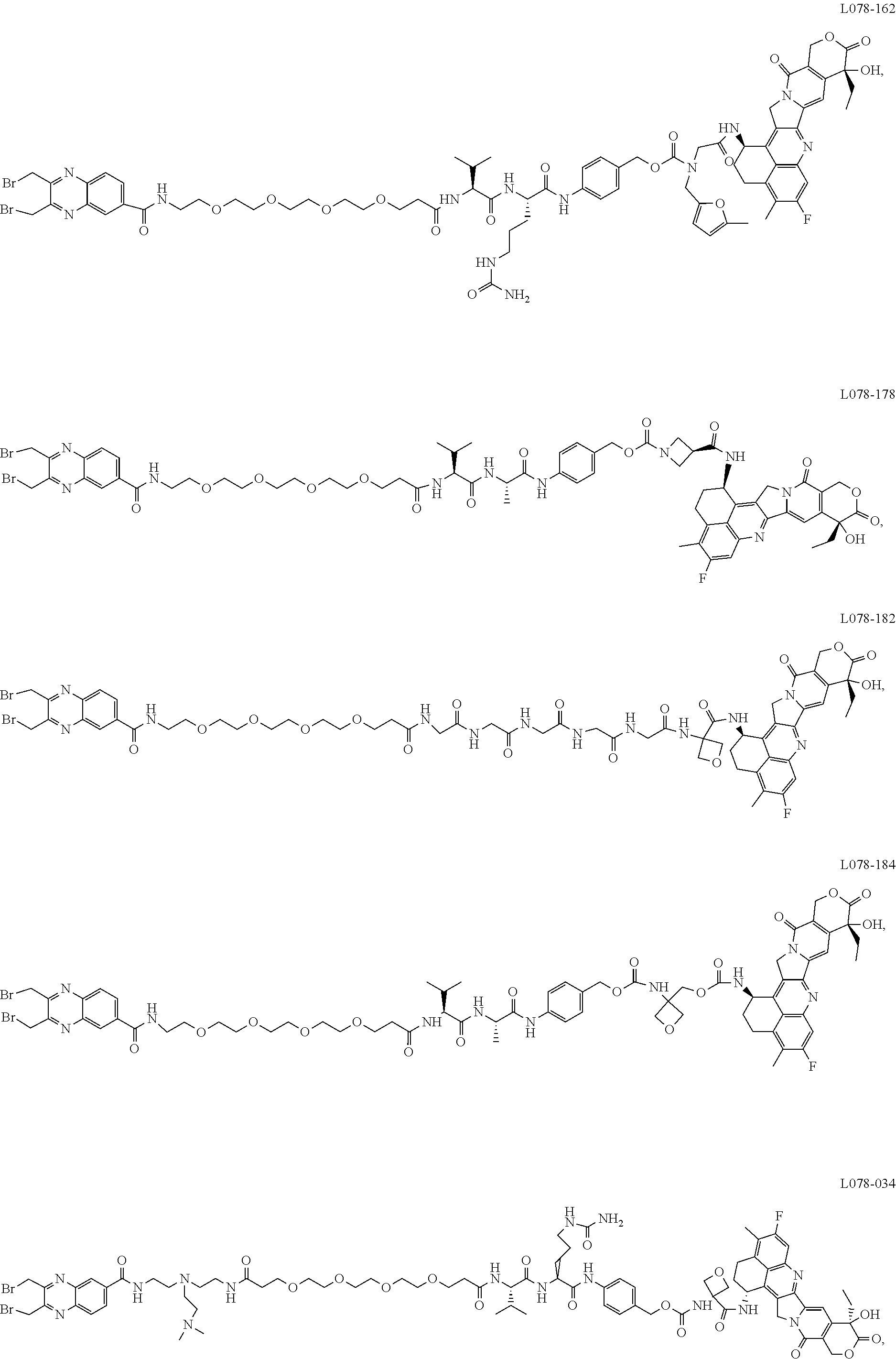 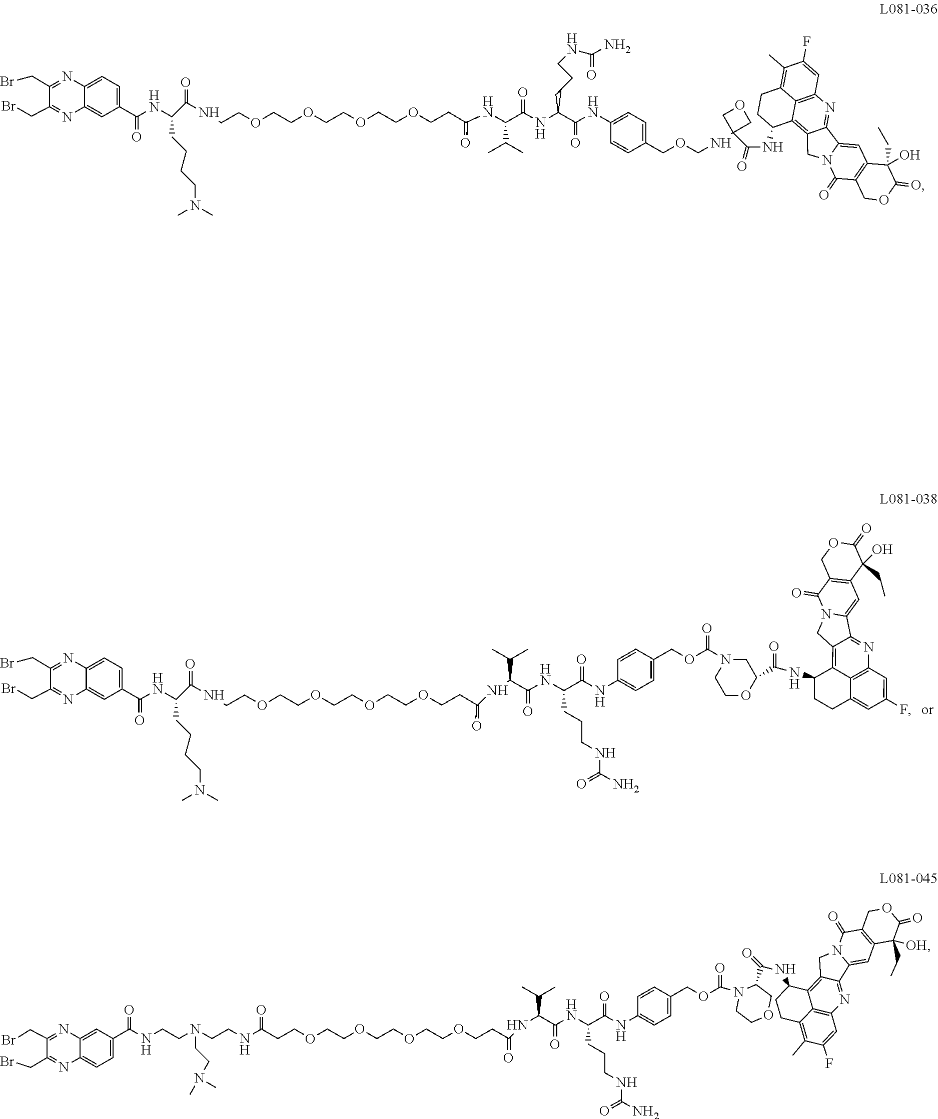
or a pharmaceutically acceptable salt thereof.

121. The method of any one of claims 69-119, wherein B-L²-D′-R¹ is: 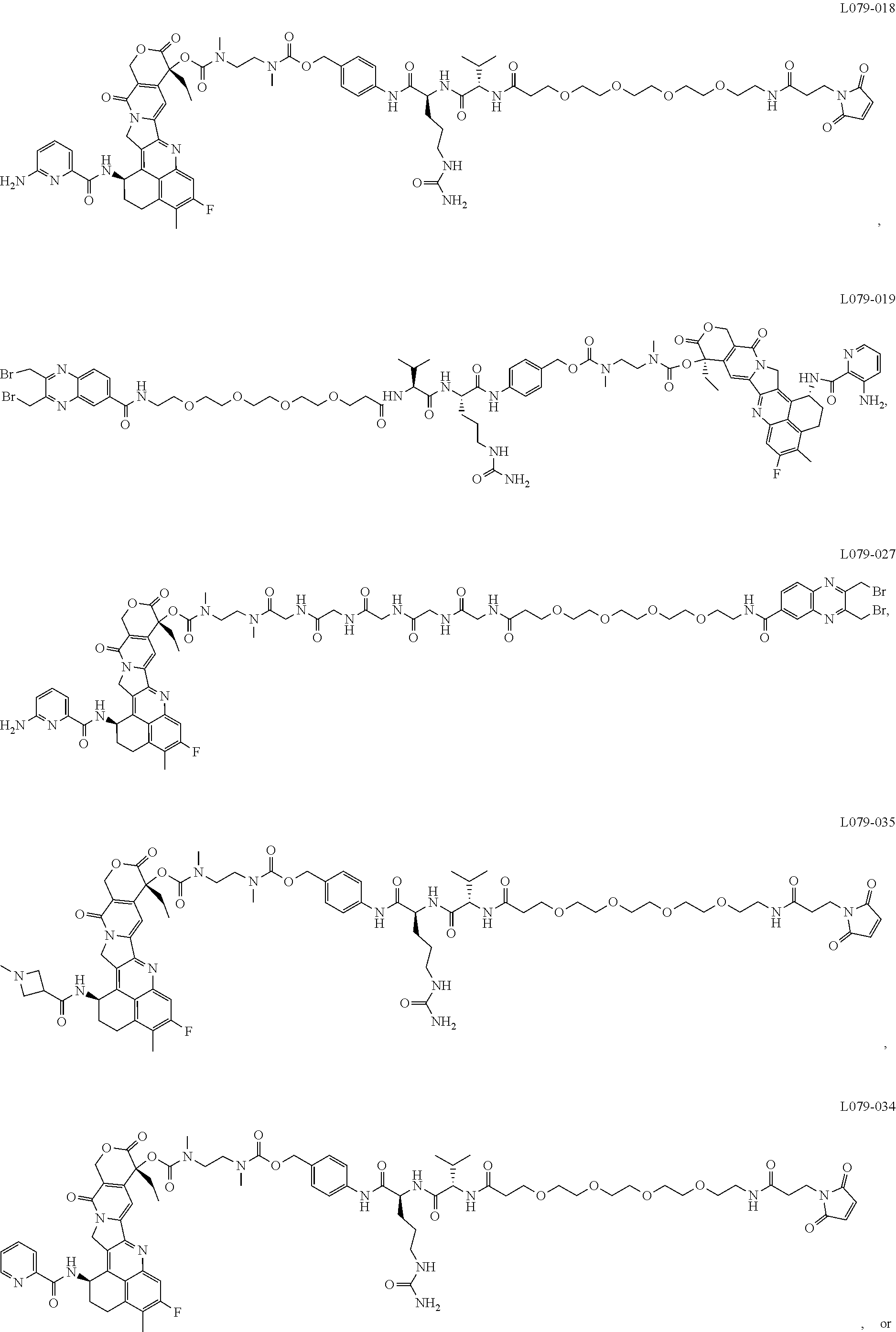 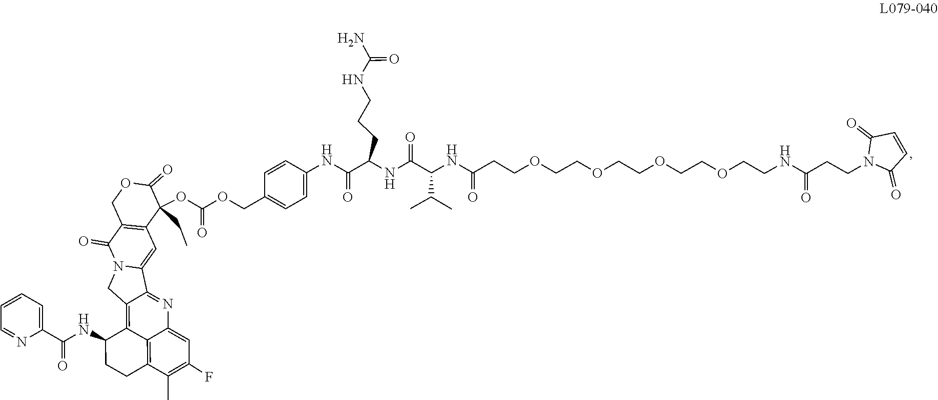 [SMILES: CC[C@]1(OC(=O)OCc2ccc(NC(=O)[C@@H](CCCNC(N)=O)NC(=O)[C@H](NC(=O)CCOCCOCCOCCOCCNC(=O)CCN3C(=O)C=CC3=O)C(C)C)cc2)C(=O)OCc2c1cc1n(c2=O)Cc2c-1nc1cc(F)c(C)c3c1c2[C@H](NC(=O)c1ccccn1)CC3]
or a pharmaceutically acceptable salt thereof.

122. A compound of formula (III): 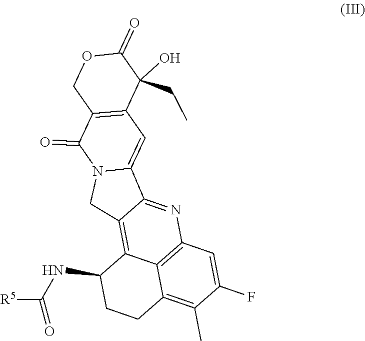
or pharmaceutically acceptable salt, solvate, hydrate, stereoisomer, or prodrug thereof, wherein:
R⁵ is a substituted or unsubstituted heterocycloalkyl, substituted or unsubstituted heteroaryl, substituted or unsubstituted —CH₂NCH₂-(heteroaryl), substituted or unsubstituted —CH₂NCH₂-(heterocycloalkyl), substituted or unsubstituted —OCH₂-(heterocycloalkyl), or substituted or unsubstituted —OCH₂-(heteroaryl).

123. The compound or pharmaceutically acceptable salt, solvate, hydrate, stereoisomer, or prodrug thereof of claim 122, wherein R⁵ is a substituted or unsubstituted heterocycloalkyl.

124. The compound or pharmaceutically acceptable salt, solvate, hydrate, stereoisomer, or prodrug thereof of claim 123, wherein R⁵ is a substituted or unsubstituted 3 to 8 membered heterocycloalkyl.

125. The compound or pharmaceutically acceptable salt, solvate, hydrate, stereoisomer, or prodrug thereof of claim 123, wherein R⁵ is a substituted or unsubstituted 3 to 6 membered heterocycloalkyl.

126. The compound or pharmaceutically acceptable salt, solvate, hydrate, stereoisomer, or prodrug thereof of claim 123, wherein R⁵ is a substituted or unsubstituted heterocyclobutyl, heterocyclopentyl or heterocyclohexyl.

127. The compound or pharmaceutically acceptable salt, solvate, hydrate, stereoisomer, or prodrug thereof of claim 122, wherein R⁵ is a substituted or unsubstituted heteroaryl.

128. The compound or pharmaceutically acceptable salt, solvate, hydrate, stereoisomer, or prodrug thereof of claim 127, wherein R⁵ is a substituted or unsubstituted 5 to 10 membered heteroaryl.

129. The compound or pharmaceutically acceptable salt, solvate, hydrate, stereoisomer, or prodrug thereof of claim 127, wherein R⁵ is a substituted or unsubstituted 5 to 9 membered heteroaryl.

130. The compound or pharmaceutically acceptable salt, solvate, hydrate, stereoisomer, or prodrug thereof of claim 127, wherein R⁵ is a substituted or unsubstituted 5 to 6 membered heteroaryl.

131. The compound or pharmaceutically acceptable salt, solvate, hydrate, stereoisomer, or prodrug thereof of claim 130, wherein R⁵ is a substituted or unsubstituted furanyl, pyrrolyl, pyridyl, pyranyl, imidazolyl, or thiazolyl.

132. The compound or pharmaceutically acceptable salt, solvate, hydrate, stereoisomer, or prodrug thereof of claim 130, wherein R⁵ is substituted or unsubstituted —CH₂NCH₂-(heteroaryl) or substituted or unsubstituted —CH₂NCH₂-(heterocycloalkyl).

133. The compound or pharmaceutically acceptable salt, solvate, hydrate, stereoisomer, or prodrug thereof of claim 130, wherein R⁵ is substituted or unsubstituted —CH₂NCH₂-(heteroaryl).

134. The compound or pharmaceutically acceptable salt, solvate, hydrate, stereoisomer, or prodrug thereof of claim 130, wherein R⁵ is substituted or unsubstituted —CH₂NCH₂-(5 to 6 membered heteroaryl).

135. A pharmaceutical composition comprising the ADC of any one of claims 1-60, or pharmaceutically acceptable salt thereof, and a pharmaceutically acceptable excipient.

136. A method of inhibiting proliferation of a HER2-expressing cell, the method comprising exposing the cell to the ADC or pharmaceutically acceptable salt thereof of any one of claims 1-60 under conditions permissive for binding of the anti-HER2 antibody of the ADC on the surface of the cell, thereby inhibiting the proliferation of the cell, optionally wherein the method is an in vitro method or an in vivo method.
